SEQUENCE LISTING (16 sequences)

SEQ ID NO: 1 - DNA, 104582 bp, synthetic construct
  source 1..104582: mol_type = other DNA
ataacttcgtatagcatacattatacgaagttatccggaaccgagtacaagcccacggtgcgcctcgccacccgcgacgacgtccccagggccgtccgcaccctcgccgccgcgttcgccgactaccccgccacgcgccacaccgtcgatccggaccgccacatcgagcgcgtgaccgagctgcaagaactcttcctcacgcgcgtcgggctcgacatcggcaaggtgtgggtcgcggacgacggagccgctgtggcggtctggaccacgccggagagcgtcgaagcgggggcggtgttcgccgagatcggcccgcgcatggccgagttgagcggttcccggctggccgcgcagcaacagatggagggcctcctggctccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccgcatgccccacgctactgcgggtttatatagacggtccccacgggatggggaaaaccaccaccacgcaactgctggtggccctgggttcgcgcgacgatatcgtctatgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggtatgccttatgccgtgaccgacgccgttctggctcctcatatcgggggggaggctggtagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggccctgccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgcgagatgggggaggctaacgagctcggcagtggagagggcagaggaagtctgctaacatgcggtgacgtcgaggagaatcctggcccaagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgacagaaaccaagacaagcgtgtccaggcacatctactttttcagcgccagaggcaggatcgaccccctggtgtacctggacgagtttctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggcagcatcagataccccgcctggaacggcatcatcagccaggaggtgctggactacctgagcagctacatcaacaggcggatcaccggttgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctggctccggcgagggcaggggaagtcttctaacatgcggggacgtggaggaaaatcccggcccagtgagcaagggtgaggaggataacatggcctctctcccagcgacacatgagttacacatctttggctccatcaacggtgtggactttgacatggtgggtcagggcaccggcaatccaaatgatggttatgaggagttaaacctgaagtccaccaagggcgacctccagttctccccctggattctggtccctcatatcgggtatggcttccatcagtacctgccctaccctgacgggatgtcgcctttccaggccgccatggtagatggctctggataccaagtccatcgcacaatgcagtttgaagatggtgcctcccttactgtgaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggaactcaagcactccaagaccgagctgaacttcaaggagtggcaaaaggcgtttaccgatgtgatgggcatggacgagctgtacaagtgaattcgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggctcgagggaagttcctatactctagaaagaataggaacttcaaaggaagtgcttcagggtgaaggaaaatgatactagatggtgacccagatatacagaaaggaacaattaacaacagaaatgatgcacatacacatgtcacattcacactcattttcttaatttcctgaagacatatggctacttgtttaaaacaaaaagtattacactgtatcattgagcttataatataggttgatgtaatatatacaacaataatagcacaatggtaggttacaagaaactacactgttagaagtgtcctttattttctggatgcagcttaatataacctgaacttcactgtgaaaagtcaaggaatcgggtttccattctttgctattgtgtaaagaaatatagccaaaagccactaggagaattaaaaccataaactaaaaaatgtttatttgacacataagaaagtagtaaaggaggaacagaaacaaaaagatatgagacaaattgaaaacgtatagcaaaattgtagaccaaaacccaaccattgcaagtgaagaaatgacatgacctgagtcacattagcaggactgctgagcactgtggggagaacagatatgggcaggaggtgagggacagtgttagtgccacaattcaggagggaaagggtggcagggactaacgggggtggtgtgaaggatgagaagggcagacagaagggctggagaggcaggaggagaggaaaaggagcaggagaaagaattctaaagcagtggaagagcctggcagagggttctttgcattcggtatttaatacattttgttggacttcctaaaaactaattggctccttatgattaaaaaaaaaagagttacaaaaataccaagtgttcagataaaatatgcacactgcttggatgtgcagagttcaggaaaacaggcagtgcttgagcgctggtgaagagcattgggactgcatggagccctcgcaactttgaggtgatgactacaggttcccggttgcaatagacagtaacaaaccctgcttctttatattcaggagatgttctggactcacacagggaaactcagggtggggaatgaagataattttaaatgcaacaacccagagtcacagatccatagtctgggaaagtaaaacttaggagctttgagagtttaattgtaatgctgttttgacacaggtcttttgcagattggaattctaatcattcagggattaccaatattgtgctacctactgtattaataaacaaaaaggaaactggtctctatgagaatctctgtgtggtggcttcagacaaaacttcgccaggtttagagagaaaacccctgtctctacacctccattcccagggcgagctcactctctggcatcaagttctccgtgatcagcttccctacacaagantccaagaggagaggtaaggagtgagaggcagggagtccagttcagggacagggattccaggaggagaagtgaaggggaagcgggtgggcgcagcctgggggtctctccctggtttccacagacagatccttgtgcaggactcaggcagacagtgtgacaaagaggctggtgtaggagaagagggatcaggacgaagtcccatgtcccggacggggctctcagggtctcaggctccgagggccgcgtctgcattggggaggcgcagcgttggggattccccactcccacgagtttcacttcttctcccaacctatgtcgggtccttcttccaggatactcgtgacgcgtccccatttcccactcccattgggtgtcgggtgtctagagaagccaatcagtgtcgccggggtcccagttctaaagtccccacgcacccacccggactcagagtctcctcagacaccgagatgcgggtcacggcaccccgaaccctcctcctgctgctctggggggccctggccctgaccgagacctgggccggtgagtgcgggtcgggagggaaatggcctctgtggggaggagcgaggggaccgcaggcgggggcgcaggacccggggagccgcgccgggaggagggtctggcgggtctcagcccctcctcgcccccaggctcccactccatgaggtatttctacaccgccatgtcccggcccggccgcggggagccccgcttcatcgcagtgggctacgtggacgacacgcagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccggtgagtgaccccggcccggggcgcaggtcacgactccccatcccccacgtacggcccgggtcgccccgagtctccgggtccgagatccgcctccctgaggccacgggacccgcccagaccctcgaccggcgagagccccaggcgcgtttacccggtttcattttcagttgaggccaaaatccccgcgggttggtcggggcggggcggggctcgggggacggggctgaccgcggggccggggccagggtctcacacttggcagacgatgtatggctgcgacctggggccggacgggcgcctcctccgcgggcataaccagttagcctacgacggcaaggattacatcgccctgaacgaggacctgagctcctggaccgcggcggacaccgcggctcagatcacccagcgcaagtgggaggcggcccgtgaggcggagcagctgagagcctacctggagggcacgtgcgtggagtggctccgcagatacctggagaacgggaaggagacgctgcagcgcgcgggtaccaggggcagtggggagccttccccatctcctataggtcgccggggatggcctcccacgagaagaggaggaaaatgggatcagcgctagaatgtcgccctcccttgaatggagaatggcatgagttttcctgagtttcctctgagggccccctcttctctctaggacaattaagggatgacgtctctgaggaaatggaggggaagacagtccctagaatactgatcaggggtcctctttgacccctgcagcagccttgggaaccgtgacttttcctctcaggccttgttctctgcctcacactcagtgtgtttggggctctgattccagcacttctgagtcactttacctccactcagctcaggagcagaagtccctgttccccgctcagagactcgaactttccaatgaataggagattatcccaggtgcctgcgtccaggctggtgtctgggttctgtgccccttccccaccccaggtgtcctgtccattctcaggctggtcacatgggtggtcctagggtgtcctatgagagatgcaaagcgcctgaattttctgactcttcccatcagaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatggggtaaggagggggatgaggggtcatatctcttctcagggaaagcaggagcccttctggagcccttcagcagggtcagggcccctcgtcttcccctcctttcccagagccatcttcccagtccaccatccccatcgtgggcattgttgctggcctggctgtcctagcagttgtggtcatcggagctgtggtcgctactgtgatgtgtaggaggaagagctcaggtagggaaggggtgaggggtggggtctgggttttcttgtcccactgggggtttcaagccccaggtagaagtgttccctgcctcattactgggaagcagcatccacacaggggctaatgcagcctgggaccctgtgtgccagcacttactcttttgtgcagcacatgtgacaatgaaggacggatgtatcaccttgatggttgtggtgttggggtcctgatttcagcattcatgagtcaggggaaggtccctgctaaggacagaccttaggagggcagttggtccaggacccacacttgctttcctcgtgtttcctgatcctgccttgggtctgtagtcatacttctggaaattccttttgggtccaagacgaggaggttcctctaagatctcatggccctgcttcctcccagtcccctcacaggacattttcttcccacaggtggaaaaggagggagctactctcaggctgcgtgtaagtggtgggggtgggagtgtggaggagctcacccaccccataattcctcctgtcccacgtctcctgcgggctctgaccaggtcctgtttttgttctactccagccagcgacagtgcccagggctctgatgtgtctctcacagcttgaaaaggtgagattcttggggtctagagtgggcgggggggggtggggtggggagggggcagaggggaaaggcctgggtaatggagattctttgattgggatgtttcgcgtgtgtggtgggctgtttagagtgtcatcacttaccatgactaaccagaatttgttcatgactgttgttttctgtagcctgagacagctgtcttgtgagggactgagatgcaggatttcttcactcctcccctttgtgacttcaagagcctctggcatctctttctgcaaaggcacctgaatgtgtctgcgtccctgttagcataatgtgaggaggtggagagacagcccacccccgtgtccactgtgacccctgttcccatgctgacctgtgtttcctccccagtcatctttcctgttccagaggggtggggctggatgtctccatctctgtctcaactttatgtgcactgagctgcaacttcttacttccctactgaaaataagaatctgaatataaatttgttttctcaaatatttgctatgagaggttgatggattaattaaataagtcaattcctggaatgtgagagagcaaataaagacctgagaactttccagaatctgcatgttcgctgtgctgagtctgttgcaggtggggtgtggagaaggctgtggggggccgagtgtggacagggcctgtgcccagttgttgttgagcccatcatgggctctatgtggttagtcctcagctgggtcaccttcactgccccattgtccttgtcccttcagcggaaacttgtccagtgggagctgtgaccacagaggctcacacatcgcccagggtggcccctgcacacgggggtctctgtgcattctgagacaaattttcagagccattcacctcctgccctgcttctagagctccttttctgctctgctctcctgccctctctccctgccctggttctagtgatcttggtgctgaatccaatcccaactcctgaatctgtaaagcagagtctaatttagacttacatttgtctgtgaaattggacccatcatcaaggactgttctttcctgaagggagaacctgattgtgtgctgcagtgtgctggggcagggggtgcggggagggggttgctgtagaaagagggatggggagggatggcctgaggctatgttaataaagatactgccttcaaaatagggaggtgctctacagtgatcattcattcaactgacctttgtcattggccagacataggacagaatggttctgcatctggggaacaccactgaagtaaaatcagaaaaatctctggccttttgtagcatatgttccagtgggaagaggcagacgatagatacactataaccagagtaaggaaggaaagtgctagaaggtggtaagtgctgtgaggcaggtgatccaggatgtgggcagtggggacagggaaggtggctgttgtgctgggtagtcagtgtgtgccttgttgcaaaggtgacttttgaggaaagatttgagagacatgaggatgtctggggaagttctttccaggcagaggaagctccagtccaaatgcactatggcaggaaggtgtttgtgttcccagaagagcaaggaggccaggagggctggacggagagaaactgaggtgaggtcagaggtgcggccagaacaggtgggcttgaggggagtggggttggatctggcctttgctctgagtgggatggggagttagaggacagttttgagcagaagagagccatgatatgacttctgttttaaaaggatctctctgacggctgtgctgagaacagaattgagaggcaagggacgagggaggcagaagggaaaacagtaggaatcaagtgcagtattccaggctggagatgtcggttaccttgactggggcgtgagcaggggaaatagtgggacgtgaggggattctggatgcatttgaagatggactcacagcatttgccaatggattgtatctgtgatgtgagaaagacgaatcaaggacacccatagttgtaaaatgagtgagtagaaggatggagctgctgtcagtggagatggggagactctggcaggagcgtcctgaagaggggacatcacaggcactcagtggaggagatgtctactaggagggcgggtgggggagctgggtggaatttggacagacaactccagagtttaggggaaaggactgggctggagaaatagatttaggaggtcacaccatatatatgagacttaaaacctcaagcatggatgaggcacaaagggagtgactgactatggaaaagaatgagcacaaggactgaaccctggacctccagttctaaggggtgtgatcagatcacacccagagcagactgcacagttctggccccatgtctagaggacactcagacaaggaaaccccatgtgcaccaggatcatctggatgtggtgctgagatcctggaagtctggagttgagcaagagattctggatttatgacaaggctggagctcatgttgctggtctccagatcacacttggagtagcaagaacaccaggatcccacacgtctgagcatcagcctcacctctagggcttgtcatataaatgattccttggtcttgtgcataattctctgagacaagggttctggggagtggcctgtgtattttctaagtccccaccagcaatcctattgctcagacagattgggagccactgagatcagtgatcagagagtgcccagggtgggtgggtggggtgggttttcaaaccctgttgaaaagaggatttttctcacagaaagaaaagggaggatgtatatcatcagttatgagaggtgatattctctgttgttctctccaccatggggtagaggccaggtagacaattcaggatgtggctctcgcacaaagaacacctctgaatgccgctctctgacactcgcccgcagactcatttctcactcacttcttggagaaaactatggaaaccaaatttctgtaatgtacacataaagttgtatattggtattgggggctagttttattgtggggaaggccacagaaccaggctggaaactacacatccaagaacagaattcacagcccaccctggatcaggtccctcctaggaacaactgcccctgctgctgagcacagacagcactgctcacacctctgacatcctggtgctggacactggaccctgaggctaggacagatgtcactgctgcacctggaaattggacatcactactgtcactcagccatctttactaaaatgctttctgcacagtcctagcctctctgtcacctcattctggctgagtctggcagtgatataggagttaagaagaaattatttagacaggtagtgagggtacgcaaggcttttaatggaaagcaggctccaaattattttcttttctaacaaggagcagcctgtaatatcaagcttcagacatagacaagtaagctggaagtttgcacgggtgaatgccagcagctgtccaataggaataggacgcctgggactgggcatgttcactatggcggctccatcttcccttctctttgccaggcacgtttatagtaaggagcagacaacatgtcaccggccaagtggaaagcccatttgcataataggatgagggtggggtgaacagccttccacacgcactatgtaaatatcacacctggtacaaccaacctgtgggccctacataaatcagacaccatctcctcaagcctgcctgtaaaatccgatgcactccattccaggctggaattcccttttgggtgcccctctctctcacaagaaggagctgtttccctttgtctttcttttgcctattaaaccctttgatcctaactcactcctcgtgtgtcagtgtccttaatcttcttggcgtgagatgacgaacacttagtatttacctgagacaatgacacctcttcagcaggtggtcatcagactggtggagcttaaaattcatgtccatgatcaactgcaggggtgactggagtgttgttgtcctcttccagggagggaggtggtgtctgtctcctctcaacattttacatgttgtaaaccaaaaataaacatcaaagccaccccttcccaatcattttaatggacaccctcagccagggtgctcaaaagttaacctgaaagagtggctcaggccaccatgggaagcaggtattgaacatgtcttattatgccctcttccctcttggaattcaggaaaagttgaccagcatttaccatcaacacagaccttcagtctgatcagaaacatttacaatctattctctttgaagcctggcacctggaggcgtcatctgtatgagaattttggactccacaaccttttatcataacccagacattcctttctattgataataacaaccaattgccaatcagaaaaattaaaaatctacttataacccagaagcactaccccgcaacccttgcttgcttcaaattgttccaactttctggaccgaaccaatgtatagcttaaatgtatttgattgatgtctcatatcttccttaaatgtgtaaaaccatgtatttgattgatgtctcatatcttccttaaatgtgtaaaaccaacctgtgcctcaaccaccttgagcacatgttctcggtctcctgagggctgtgtcatgggctgtggtcactcatttttggctcagaataaatctctttaaatatgttactgcgtttgactcttttcattgacagtgtggaattttgatgtagtaagcgggttcaagtgctggagtgtgaggggtggggaaaagtgacaaattttaattcttggagcagtgatccaggacgagaactttatctagtacccagtaggcacgaagagtggctgaatgaaccagtgactaataaataacatatttccacccattcccttgaggataggtttacatcaagtttttgtctatcccagtttatagtctagattattggagtggcattctgtaatgatgaaatgattattcaaacctcttccctcagcatgactcttactaggattaaaactcccaggtttgtaagatatcagaggttctggttccacacgatgttttatattaatatttatattttcttgtgtgtggaaatatttctggggagagaatctctaacatttattagtttttatgtgacaccctcaaaggcctaaaaaatcctaacttctagattaaaggtaggaccactccacgtctagacaatgaacagctggtagcaacctgtgtttagggacatgaaacaaatgcttacatttgtttatcttttcctctgggaccaaagaaggtagaggtttacttgcttactcttactataagtttctctctgtgggatggtgatggggttttcttccacccaaataactttctgattctccaacagcagctgggagtcccacaatttgactcaattctgacactaactacctggagttcgcctcagactctacaggtttaccagctcagtcctacaagtctgtcctcacttcagaagcaagtcacaagtatcgggtcccaggttacctgcacttctgtctaacatggctgcaaagtcaggggtttctcacctcccaaggattgagaattcttaaactaactcaaagaactcaggaagttgctatagccgtgtaaaccaaaaaacgtctgagacaggtctcagtcagtttagaagtttattttgccaagctgaagacgcaccttggaaacagagacacaaatcattgtagcatctgtggcccatgctctttcctaacaggcttttgaggacttcaatatgtacaggggaaagagcagccaggagaggaaaaaaatagtcaactatgcattcatctcatgctcagtaaatctgcattttaaataagataaagtaaacattgggtggaaaagtcaaatatgcgtttgactctgggtgggtggagggatgatttctagtcttgtctttgtcctgtacctgtgaagataagctgttaatttacattgtcaggggaaaattcaacaaaactgttttagggtaaacacgttggggcccacaaggaattatcttgtgagcagtttgtgagggaggccacctggggagatatgtggtcttctatctttgcagttatttgtttaggaacgaaagaaaggcagtttttccatcagtctccaaacttaatttttccttttggtatagtgaatttggagtcctgaaattttattttcttttcacactcagtattgcagttttttattgttatagcttaaatccccacctttttggcacctgggaccggtttcatggaagacagtttttccatggactagtggaggtggtggagggcagagggaaatggtttcggatgaaactgttgtacctcagatgatcaggcattagcgcttcataaggagcatgcaacctagatctctcgcatgcagagttcacaatagggttcgtgcttctatgagaatctaattccgccactgatctgacaggaggcggaactctggtgtcatgctcatgctctctcacccactgatcacctcctgctgtgcagtctggttcctaccaggccaggaacctgtaccagtccgcagcccgggggttaaggacccctggtttaaattatgccactagagcagaactcctgagactggataacttataacaaacagaaatttgtctcgttcatggtgtcgaggatgagaattcccagatcaagggactctatctggtagaaaacaaaaggacaagagagggtgagaggtggggggaaaaggaagtcaaactcacagcctcaggtccttttatcatgagccttagtccatgaatgaaggcagagctctgatggtccaatcacctctcaaaagtcccatttcttaaaaatgttgcatttgggatgaaatatccaacagatgcttttttggggaaacattcaaaccatagcagttataaaggataccaaggagcagccagatgaagaggtgcacagggtgaggcctggaagggctctgagcacaggagcctctgtccccatggagatggggctcaccatcctcccagcatgtggacacacagatgtgctctccgaaacccaatctccagaggtttctatggaaatttcattgtggaggcaagactaattaaatcactggtcattgtgtgactgaactcaacctcgaacctctctttccttcccagactttagggatggggttaacagttccaatcctctaatcacaacttcgtgtttctggcaaccagcccccatcctgaagctatctaggagcccctcaccccatagtcatctcattgccatattgaactcatgtttatcactgaggagattgcaaatgttttagaacctggttgtcaggaaacagggaaaagaccaaatatttttattggaccacagatcacaccctggttttgaccacattatctctttttataatatttattattttattttagagataatctcttgctctgttgcccagggtagaatgcagtggcacaatcatagctcactctaacctcgaattcctggactcaagtgatccttctgcctcagcctcctgagtagatagcattacaggtgcacaccaacatgcccaactaatttttaaatattttgtaggaatgggtctcactattctgcccagtctggtctcaaactcctggacttaaacaatgttcctaccattgcctcccaaactgatggaattataagtgtgagccactgtgcctggccaatcacagatctcctacaccagaagaatcataacagtgaaaagatcctggcacattaccagaatcctctttggtcattaacaatgatcccagtgcaatccataaacaggaatagtttcagcaacatatggctgcaccctttcaggcgtctggtaaaattgagctaagagacaatatcatctcttgcttatacctttttcaagtagttcatgtaacattggattttcctcattacataacccatttattcattcatttaccctcagctactatttctccttcccttcatttatactaaacatttataattttggaaggaacactagcttctgctgctgtgctggctgagactgcagggaccaatagagttctagcaagtgtctcccgtcatccattccagttcatagagggtagggttatacaggtagagaactggttggctatctgatcccaggcaatatagctgcattcagtgttagccccaactttgccagatgcagtgaaggcacaacctacttctacaggcccttaggaattattacataatggacataaaatatatttacagtttcttgcttaggaataattcctgtttctgaactctatttgcatccctagtcctgggaccactgcatcaggtatgagaaaagcaagttgaggtgaattacagtcatcattccagtgtcctcccaccttggggaggaatatatgtagatcataccagcaccttcccctgatccaccaggaaaagagaggatactctcatgtcaagtgtgagcacactcatgaaggtgtataagcccaccctttgtgcttgtttccccactctttcctttacacacccaattgactattttaattctctatggaactcttcctctgaggaggatatctctctgtgaccattgttggacattttgagcaggtgcattttctggtgtaaagaaatatgactcggtggtccacgtggttgcagtacctcccattcaagttcttttgtaacactcgagcatttgcatctgagataaagcagggacctcttttagaagcctgccggggcctcccaatctggaaataaagaaaaaccttcagttccttcaagagaaattccaggcacctagccagccctaaaaagtaagtcggtgaccttataagcaagaaggaatgatagcttgaaacaatagccaaggaagttagagtcacaagatgtttcattctctatagaaactaaaaataacatcttaacatatgtccctgagttgtttttcagaaacatggattccctcctaatagatccactgacagggagaccttataaaggggaaactgagaactgaactctgaccgccgttctttgttctaaatttcttcctgagggacctggagagagtcaagcccacaggccaggcctgaacattcctctctactgcccccaactgtgtaaggaagctgtgtaaagaagctttgcttccttatctatcacaaatcagagaatctttaaatctacctaagacctgtaagctccctcatcaggatattctaccttttaaggccaaagcagtgtaacttgtatgcattgatttatgatgttgcctgtaactctgctttcccgaaatttgcccctgcctttaaaaattcttgcttgtgagccatcaaaaangttgggtcttaagcgtgagctgcctgattctccttgtttggcgccctacacataaatgccctcctttgtcccactacaatctcagtgtggatgtttgcttttactgccccaggtgagtggaccccagttcagtttggtaacacacctaccaccagctgaacaaagcccactccagggtgaggatctattcctgccaagacccgtttgctgcttcctggggctactggtatcagtctaacttgccagctatgtattttcaaggccctcccataaaagaatctgccacatagccatatgctatctctctttttttttgttaaagaaaggacttcttatgtgtattttgtgcctgtgatgtgtggaatgcaagaggactatgtcttgattccacctatctctgcattgctacagccccaatgtctacttatttcatggactcaggtgatcaccacaagcaaacctggattttttgttgttgttgtttttgagatgtggtcttgatatgtttccctggctggcctcaaatgttgctcaggttaaactctaactcctggactcaagatatccttctgcctcagccgctctcatagctggaattacaagtgcacctggcaaacacttggagataaattcttgttgatttcaatcactttccaaacctgaaagggagttattctgaaggacactgaacagcactgaggggacggtgctaaaccattcatgagaaactgacctcgtgatccaaccacctccctccctccaggccccacctataatgctggggatcacatctcgacatgagatctgcacaagaacaacatccaatctatatcaggcatcaacagatcttacattaatgaacccctcaaattaccatagtgatttctgtgggacttggctcatatgggcattcttttcataggcaagtttttcattgctttcctgattaattatacggccaggccattagtcactccctgtaagtcagtaaaaactaaaacgtaggagctttatcattgttcaattcttccaccactgctaagaaaacaccatgcaattaagtccacaacagcttttctgtttttaccttctttgatcaaagaggtggacttccaaacagtatgttgtccattcatcttgcaaatgctgtacacaaaccaaccagctgttagtgggtcagttgactgatactgcagttcaaatcccagcgctgtctgctacagagttccatcttgctcaagagaggtcagtgttttgtttaatttacaccttcagtgaattgaatgaagcccactcacattatagacattaatctactttattcaaagtctggctatttatgtgttaatctcatccaacaaacatcctcacaaatcatccagaataatgcttgaccacctctccaggcacgatgctttacccaagatgaccaataaatttttttttattattatactttaagttctagggtacatgtgtacaacgtgcaggtttgttacatatgtatacatgtgccatgttggtctgctgcacctgttaactcattatttacattaggtatttctcctagtactatccctccccactgcccccaccccacgacaggccccggtgtgtgatgttcccccctgtgtccaagtgttctcattgttcaattcccacctatgagtgagaacatgcggtgtttggttttctgtccttgtgatagtttgaacaatgagaacacttggacacttgtgatagtttgctcagaatgatggtttccagcttcatccacgtccctgcaaaggacatgaactcatcctttttatggctgcatagtattccatggtgtatatgtgacacattttctcccagcacctgttgtttcctgactttttaatgatcgccattcttactggtgtgagatgggataccattgtggttttgatttgcatttctctgatggccagtgatgatgagcattttttcatgtgtctgttggctgcataaatgtcttctttggagaagtgtctgttcatatcctttgcccactttttgatggggttgtttgattttttcttgtaaatttgtttgagttccttgtagattctggatattagccctttgttagatgggtagattgtaaatattttctcccattttgtaggttgcctgttcactgtgatggtagtttcttttgcagtgcagaagctctttagtttaattagatcccatttgtcaattttggtttttgatgccattgcttttggtgttttagtcatgaagtccttgcccatgcctatggcctgaaaggtattgcctaggttttcttctagggtttttatggttttagggatgatcagtaaaattaattatgacattcttcaatataagaatactctgaggaaattagcctgtgatctaataaaacaaggagcaaacaactgtttctaaaaatttataacaaatagtcaaaaagccctcgctagagattgtttctcacattcactctataattctgggtaaaaaaaaaagcacgtatttgttgaaaaccacagaagaagagaaaattgaaaaagcagaaagtgaaaaaaataagaataataataaagatgacagcagttatttcattggaaacaatgcaaatgaggagacagatgagcaacatctttagagtcctaaaaggaaaatgctgtgaaactagattctattcccagaaaataatacctttttcaaatatatatgtatagttttaattgacaaagtataatacattcatgagatattgtttgatgtattgatatatgtgtaccctatgtaatgattaaattaagctaacacaccttgaatatatctatcaaaaaataaagggattttaggccaggcatggtggctcatgtctgtcatcccaacactttgggaggccaaggtaggtagatcacgaagtcaggagtcaaaaccagcctgaccaacatggtgaaacctcgtctctactaaaaatacaaaaattagctaggcatggtggcatgctcctgtaattcccagctactcaggaggctgaggcaggagaatcacttgaacatgggaggcggaggttgcagtaggccaatatcacaccattgcactccagcctgggcagtagagtgagactctgtctcaaaaataaacaaacaaacaaacaaacaaacaaacaaatggattttagacatacagggagtgaaaaaataaccagcagacctaaactataataaatgtttcagtcctataggcaaaaagaaaattaagggactagttggcacagcatttaacgatgaagagacagcttcatcgccccctcccactgagacagagaacacagccccagggcctgggaattcctcaacccaatccaccaccttgggcacctgtgcactcctcctgggtacctgaggttggatgcaatcaccttagctatcacctacctgcaaacaccacctatgggcatcccacccagcctatcgcatctgctgctaacaatgcacactgcttgggacccagagaagcagcccacgactgccactgccaagctggttgcccaggagctgaagtccccagcacctaccgagtcaaccactgccactactgccatccaagcaagccacctggaggtccaggaatcagcccaccagtaaccaccaatacaaatgccagtatataccaccttggggcacaaagataggaacactcagcacaccactgccaccactggagcctgaagactgtctcatgtggcatcccagtccccagcacattttcaccacagcctcactaataaccacaccctaatgcacagaagaaatcacagataccattaacactgtctacagccaaagaaattatacagagaccatagtactctactatgtactacacccagaatcaaatccaaagtaccccaggcaaccaacaccatagactcatcttcaggaaaatgctctccactatgaaacaaaattcaaaaataggaagaactgactgttatatcagatgcaaaaatattaatgttaggacacagaaacatgaaaaagcaaaaaagaaaaaaaaagccgcaaaggaagacaatagatcttaatcaaaaagaaattttcaagattctagataaaggactcaaaacatttattttacagaagttcagtgagatacaaaagaattctgaaaaaaaaaatacaaagaaaacataaaaataattcaggatacaaataagaagtttacaaaagatatagatttaaaaaaaaacaacagaaattctggaattaaagaatacatgacatgaaattccaaatacatttgaaagcttcaacaatatactaggtcaggcagaaggaagaattccaaaacttgaagatatacattgtgaaataattcaataagacaaaaacacacacaaaaaaagaatgagcaaagcctttgtgacatttggtacaatataaacaaccaaatattcaaattattggtatcctcaagagcgaagagacaaagaacagattagaaaacctatttaatgaaagaatagatgaataattcccaactctagcaagtgatttagacattcagatacaaaatattcaacaattctgagaaaaatacaatgcaaaaagttcctctccatggcatataatcagactttctatagtataaggtaaaatgcaaatcctaaaaatagcaagaggaaagtgtttagtcacctataaaggaaaccccatcagactaacagcagatttctttttcttttttctttcttttttttttttttgagacggagtctcgctgtgctgccaggctggagtgcagtggcgtgatctcggttcactgcaacctctgccccctgggttcaggtgattctcctccctcagcctcccaagtagctgggatgacaggcacatgccaccacacacagctaatttttttttttttgtatttttagtagagacaggatttcaccaagttggccaggatggtctcaatctcttgacatcatgatccgcccacctcagcctcccaaagtgctgggattacaggtgtgagccactgtgcccagctctaacagcagatttcttngcagaaatcttacaggccagaagagaataggatagcataatcaaagtcctgaaagaaaagaactgccaagccaagagtaataaaccaaacataaattattcttcataattgaaaaagaaattatctttccaagacaagcaaatgctgaaaggcttcattaccactagacagggcctacaggaaatgctcaagaaagtcctaaacctggaattgaaaggacaacattttcaacatttccacattatcaccatgagaacatacacaagtataaaactcactggtaaagcaatcacacaaaagaggaagagaaagttctcagatggtcctactacagaaatccaccaaagcataatgacaagcaataagagaaaaagacaataacaaaagacatataaaacaaccagagaacaacaatgtgaaaggaacaaagctgttgggaacaggaccccaaaaatctggccataaactggccccaaaactggccataaacaaaatctctgcagcactgtgacatgttaatgatggccatgacgcccacactggaaggttgtgggtttaccagaatgagggcaaggaacacctggcccactcagggcagaaaactgcttcaagttgttcttcaaccaccaacaatagcatgagcgatctgtgccttaaagacatgctcctgctgcagataactagccaaacctatccttttattttggcccatccctttgtttcccataaggaatacttttagttaatctataatctatagaaacaatgcttatcactggcttgagttaataaatacgggggtaaatctctattcgaggctgtcagctctgaaggctgggagacccctgatttcccacttcacacctgtatatttctgtgtgtgtgtctttaatttctctagcgccgctgggttagggtctccccagctgagttggtctcagcacaaagcctcacatattaataaaaatctggaatgtaaacagattaaattctccacttataagatgtagaatggctgaatgggtagaaaacatgatctaactacattctgcttacaaaaaaactcactggtaataccacctcaccacaatcatagagacagaaggtaaaagaatggcaacaggttatctatcgaaaccaaaagtgagcaggagtagctatacttatagcagataaaataaactgtaagtcaagaacaatttttttaaaagataaggaaggccatcacataatcataaagagatccatccaagaagagaatagaacaattctaaatatatatgcactaaacattggagcanctagattcataaaacaaatattactacatctaacaagagagatagactccaatacaataatagtgggggacttcaacatcccaacctcagcataagacagatcacctacacagaaaatcaagaaagaaacattggatttaaactgaattttacaccaaataaagctaacagatatttacagagcattctgtctaacactacagaatatacactcttttcatgaacacatggaacattttccaggagagagcctatgttaagccacaaaacaagtctcaacaaattttcaaaaattgaaatcatattaaatatcttctctggccacaatggaataaaactagaaatatacaccaagagaaatgttggcaacaagaccaatacatggaaattaaacaacctgctcctgaataaccattgagtcaacagagaaattaagatgtaaatcaaaaagtttcttgaaataaatgaaaatggaaacacaacataccaaaacctgtgcgatacagctaaacagggctaaaagggaggtttatagcaataaatacccacctaagaaaagtagaaacattacaaattaacaatataacaatgaacctcatggaaccagaaaagcaagaaaaaaaatatacaaaattagcagaaaagaaataataaagatcatagtagaaataaagacttttaaaaaccatgtaaatgatcaacaaagtgaaaagtttgttcttcaaaaacataaacaaaattgataaaccactagctaaacaaacccagaaaaaaagagagaaaatttattaagaataaaagttcctcttcaaagttttcctttttggtttagaatgataagtcttaagaagatagttcctcttaaaagcttcctttaagtttccctcgctcttcatactaacaaatcttctattttactaatgactctttattgtgccccaaaggagttgttacatacagtaaanaaataaacacattctatgttcttgtacttgaaccaaggcctaactcctttgttccctagtctgcctggatctgcctggacacgcccagacatgtcacagcttgcagcctatgctctttccttatttggaaatattattgtcttcctggtttcccataagcgaccccctcctttccttggttccccattgcgcctttaccttatttgggaaagtttaagtttttagccaactgggatcagtttagattgtgtggtccagttccagccaatggagacaggacacaatagtagaaacaaattgcgttaggaataaaaacccctgctttcctttgttctggttgctctcgtggcaatcaggcttacaggtagcagccttctgcagaagtaaaaattgccttgctgagaaatcctttgatcaagtgttcatttcctttgtgactccaagctttatttctaactattttatgggggctcttccaggattcccattctcctatgggaaggggtctttggtcaccccatctaggggaggtgtaccccactgcagtggccccagggacgggagattgagactacccattgtgatgaataaattcaggctctcagcaatgtggaaggagaaggcccacaagtaccacgatgaccagctaactgtgcagagaccaaggtaaaaaaagtcacaagggcaacagtatttccttggtggtcgggatattctggaggttaaagggtgtgaaaggtaacaagcactactgctgtgcagagtgagtgagttcaatctgtggttccgtggtcacctcatgtggcttatgatggccctcttgtagggatcctgacattggggttcatacagtcctgctgatgctaaggggagcctaatttcctctgggaaagcagccagagtggatgaagcaaaaggagaagattgcaagaaacctccagggtgggggttaaacctccagggaaggataggcaagaaatctctagttcgagggactgagcctaaccagtattcaacatgggaaataccctgagtaagacagaaggtaaaaaggaaaaggcaaacagtgaaatcacctctgatagtcctttaggactcatgttaaaatattggaaggatacaagaggactaaacataagaaaaagcaacagatgataaagtattattgtttcatttggaccaaggaacctattctaaagccttcagttttctggccaaaattcgggtcaaatgaggcttggacttgccaacttttaatagaatgtgtaaatgataagagtcctgtttcccaggaggaaatacattatgctctgtgttggcagcaagggcccatcctcctttgtcatctaaaaaccaaaggagacaagccagaaactacttctcatgaaattaataccccttactccaagaaccccaccaacacgtgggacgccttagaccatcttcccccaacaaaacttcttccccccacccagtagaccaacatcccccacctcccccttcagtagaaataacagtcccagacctttcccctactcagacattttttccctccttttaaccctgattcttggggtcatccccagcttgaatgccctccctcaggaagacttcaacgtgagttagaacaatgtaaaaatgatatccaaaacttcccttttcccccttcctctaggggtttggctacaaacctcttcccactgagggaagtgcctctaggaggagcaggcattggctttttaaatgctcccctgaccagttcggaagtccagagcttgaagagggaactcaagccactcttagatgatccctatggagtggcagatcaaattgatcaatttttagcaccccagttgtatactgtggctgagtttatgtccatcataggcatccttttttcaggggaagaaaggagcatgatgcatagggccgctatgaccatttgggagtgtgagcatcctcccggtcaaaacgtccctgcagccaaacagaaatttcctgctcaagatccccagtgggataacaacaacatagctcataggaggaatatgagagacctgcaagaaatgataataaaaggaattagataatcagtaccctgcacccagaatatgaccaaagcttttaatatacagcaaggaaaagaagaggggcctatggaatattttaataggcttaaggaacaaatgaggaaatatgctggtttagatggaggagacccacttggacaaggaatgttaaaagcttcactttgtcacaaatagctggccagacgttaccaagaaattacaaaagatagaaaattgaaaggactgtcccataaaggaactcttaagggaggcccaaaaggtgtatgtacgaagggatgaagaaaagcaaaagcacaaggcaaagattatgctgttcttaaagcagggaactccccaatagacagctcaaagaaatatcgccggtaaatcttttaggtacccgactgccagactctataatggaggtaaaggaatcaaaccagggaacaagggaataaaaagaggaatagggcagaataaatgcttcaaatgtgggaaggaaggtcactttaaagagaatgccctgaatggaaaaaagaaaaagaaatcacccacttatgatctttgaggaggaatggggggccaggggctctactctttttacctcgagtcccaccaagactccttgataaatttagaggtgggacccagacctttttaattgactcaggagctgctgattcctcagtttgttatcttccccctggtgtaacttggtcacaagaagaactttttattttgggagcaaaaggggaggggtttaaagcaaaagtcttagaagaaacaaaaattaaatatcagaaccactcagtaaatattaaacttctgttaatcctggaggcaggaacaaacctattaggaagagatttaatgctaaaattaaacctaggcctttgtgttaatcaaggaaatctcctcccctccttaaatttgcttgctactctaaatgagggatacatccatccagatgtatggtcaaaggaaggaaactgaggaaagttacaaatttccccaatccaagttaaattgaaaaacctggggcaagaggtaaaaagaaaacaatatccaattcccttagaagctaagatcaatttaaagcccacaatagaaagccttctccatggtggactcattgaaccctgtatgtctccttataacactcccatactgcctgtgaaaaagccagaggggtcatatcggctagtgcaagatcttcgatctattaatcaaatagttcaaacaactcactctgtcgatcctaatccttatgctattattagtaaaattccttacaaccatgagtggttcacagtaatagatctaaaggatgccttctgggcttgcccactagcaaagatagactttgaatgggaggaccctcactctggttgaaaataacagtatcgatggacagttttgccccagggatttacagaatctccaaatttgtttggtcaaatactagaacagatcatagaaaagttttacaaaccaccatatatatgcctgctccaatacatggataatatccttatttcaggggaggatagacaaaaagtagaaggattttcaataggttttttaaaaataatttgcaactggaggggttatgagtgtcaaaaaagcaaacttcggtttgtagaacctgaagtcaagtatttaggggacttaatcagtaagggcaaatggagaattggatttgaatggattgaaggcatcatatccttcatatgatatcctgagacaaagaaggaacttagaaagtttttagggttagtagggtattgttgcttatagagagactcttatgctttagtaaccaaacctctacataaaaaaaacttacacaggatgaattggacccccttatatggttttacaagaaatacagcagataaaaaaattaaaagagttactagtgacagcccatgttctagccttaccctctttagaattgccctttcatctttttgttaatgtaggtaatgaagtagccttaggggtacttactcaaacacacggaggccattggcaacccatagccttcttgtcaaaaattcttcacccagtagcccatggatggcccgagtgtgtcccatcagtggcagcaacagccttgctcatagaggagagcagaaaattaacctttggggaaatcttatcattagtactcctcaacaagtgagaacaattctcaatcagaaggcaggaaggtggcttattgattcaagaattttaaagtatgaagccatcttgttagaaagagatgatctaatcttaaccactgatgactcaattaatccagctgctttcttaacaggaaaccccaacccacagacccctgatctttgcccagagcacagatgtttagatttaattagttatcaaacagaagttagacctgatctaaatgaaacaccctttcatactgggaaacacctgtttgtagatggttcctcccgagtaatcaaaggaaaaaggcataacgagtattcaacgatagatggagataccctcacagaaatagagtctgaaaggttaccaaatgactggtctgcacaaacatgtgagttgttcacattaaatcaggccttaaaatttctgcaaatcaagaagcaacaatctatacagactccaggtatgcctttggagtagtccatacttttgaaaaaatttggactgagcggggcctcattaacagcaaaggtcaaattttagaccatagggacctaataatacaagtactagaaaatttacaattgccaaaagaaatagctgttgtacatgtcccacgacatcaaaggaatcctttatttgaaggccagggaaataatctcactgaccaaatagccaagcaagctgcctcttctccagcagaacccatttttcgactaaccccttgtcttccatcttcagctgcactccctatcttctctcaagcagatcaggaaaaactgaaaaaaataggagctgaagaaaactcaaaaggaaagtgggtattaccagatggaagggaaatgttacccaaactttatgagggaaatattgtcacagcttcatcaaggatgtcactggggtcctcaaaccatgtgtgatgcagtccttagggtttatgggtgcataagaatatacaccctcgccaagcaaatggtggaaagttatatactgtgcagaaaatctaataagcagaccctcaaaagacaacctcccggagggagaaatcctgggttaaggcgatttcaaagtgtccaagttgactctactgaaatgcccccagtaggccgccttaagtatttaccggtaatagtagatcatcttacccactgggtagaagccatcccctttccaagttcaacagccagtaatgtggtcaaagctctgttagaacatatcatacccaggtttggaataatagagaacattgattcagataatgggacccaatttaccgtgtacattattaaaagactaatccaagtattagggataaaatgggaatatcatacttcctggcctccaccttcatctgggagagtagaaagaatgaatcaaactttaaagagtcatctaaccaaattaattttagaaactcgcctaccatggacaaaatctcttcccattgctttactaagaatccaaacttctcctcggagggatcttggcttgtctccttatgaaatgctttatgggttaccttatctaaacactactactgaccttcctatgatcgaaacaaaagatcagttccttagaaattatgtatttggtctgtcttccactctttcgtccctcaggactcaaggtctggtagcacagactccaccccttgaattctgggcccaccaacatcaacccggagatcacacgtccttatcaaaggctgaaaagagggaaagcttgagcctacctgggaaggaccctatctggtgctcacaacaactgaaacagtagttcggaccactgagaaagggtggacccattatactcgagtcaagtaggcgtcaccttctccagagacatggaccattattccgtcaactcccaccagagtaacgataaaaagaaaaacttaatctatctctttttcttcttttctttcccttaactgcccccatctcatcattaatgtaactagatcaagtttgcccaaaattattacctttgatgcttctctagtcatgccttgtggaaatttacaggaccaaagacagcttactgcctcagataaatatctttgtccctcttgaacttcctcagattggaaagatataacaaattcttgtaaagatcatgataaccccaaaaatagtagctgtatgaactggtgggaatgggactcttgtccccttaaaacagagtccctatgctatacttggtctaatgtcctatggaacaccaaaggtccaggctggactgctcccaccagtctttgccagtccttaaagccatatatccattttacaaaagggattgcctcttctaattgtccatataaccagtgtaatcctattcggcttactattactattgcaacatcccaaaactctttcccttcattaagtcatatttatagcataggggccaacatcttgggaaaggatcctaggggaatttttgaaatctgctttattgccccttccacctctctaacacatcttcctcaaaccatcaccccacctatacccagtgacaaaaccaaagtagctatagtagaagtaaaagacctgaggtaaactctagccactgaaacagggtctcaggatgcaaatgcctggctggaatggattaaatattccatccacactcaaaataagagcgattgttatgcttgggcacagggcagaccagaggcccaaactgtcccctttccactcagatggtcttgtaatagaccagatatggactgcatggtagctctgctccaagacctcacagcctggggtaataaatcgtgtcaaagactttctttgctgttccctgagatcagcccccaaaggccattcagcctccatcttctgggaccaaatttgctttgtgtctctcacaacagggagaaaacttggctttccttggaaacctgacaggatgcagtgaggtcaagcacttccaagagctaacccatcagtcctcccttatccatccccgagcagatgtatggtggtattgtggtggacctttgtgggagactctgccaaataactggagtggtacttgcactctaattcaattggctatctctttcaccctgacatttcattcacaaaaatattaacaactaggcatcgtagtacaagaactgttcctgtgagtcttttgatcccaacgtttatatagatgctattggagtaccagatgaatttaaagcttgaaaccaaatagctgcagaatttaagtcattattcaggtgggtgactatcaataaaaatatagattggatagccaggcgcagtggctcacgtctgtaatcgcagcactttgggaggccaaggcgggcagatcacgaggtcaggagtttgagaccagcctgaccaacatagtgaaaccctgtttctactaaaaatacaaaaattagctgagtgtggtggcacgcacctgtaatcccagctactcaggagagtcacttcaacctgggagttggaggttgcagtgagccgagatcacaccactgcactccagcctgggcgacagactgagactccatctcgaaaaaaaaaatgtaggttgggtaaattatatttactacaatcagcaatgattcattaattgcacaagggatgctatgagggaatagctgagcaactaggacccaccagtcagatgacttgggaaaatagaacagcactagacatgatactagcagaaaaaggcaaggtttgtgttatgattggaactcaatgttgtacttttatccttaataatactgcccctgatcgaactataataaaagcactacaaggtcttactgcttcatcaaatgaactagctaaaaactctggaataaatgatcctttcactagcttaatggaaaggtggtttgctaagtggaaaggactcatgtcctcaattctcacctcactggctctcataatcagtgtacttatccttgtagaatgttgtattatatcttgcattcatggattagtgcaaaggcttatagaaaaggctcttaccaaaacccctttcaattctcctcacccttactcagataagttgttctttctaactgattaagaagagcaacaaaatcaagatatgttaaggagatttgaagaggaagaactgtaaaatcaagagggggaaactgtaaagaataaaagtacctctccaaagttttcctttttggttaagaatgataagtgttaagaagatagttcctcttaaaatctttctttaagtttcccttactcttcatactaacaaatcttctaatttactaatgattctttattgtgctccaagtagttgttacatacagtaaagaaataaacacattctatgttcttgtacttgaaccaaggcctaactcctagtctgcctggatctgaccagacatgcccagacatgtcccagcttgcagcctatgctcttgccttatttggaaatcttattgtcttcctggtttcccataagtgaccccctcctttcctttgttccccattgcacctttaccttatttaggaaagttaaagtttttagccaaccgggatcagtttagactgtgcggtccagctccagccaatggacacaggacacaatagtagaaacaacttgcgttaggaataaaaacccctgctttcccttgttctgggtgctctcgtggcaatcaggcttacaggtagcacccttctgcagaagtaaaaattgccttgctgagaaatactttgttcaagtgctcattttctttgtgacttcgagctttatttctaacagactcaaacaaaatcagaaaagaaaagagaaacattacaactgatattgcaaaagtacaaaagaccatcagagactattatgaacaattgtgtactgacaaactggaaaacctggaggaaatggataaattcctggaaacaaacaacctgccaagattacattaggaagaatagaaaacctgaagccctaacatacatgaaccattctcacagtggtcccagtgggcccctgacctctctgtgattatttcctcagtgcttgtttgcataattgaaatagacctactcagcaacagccaaaatccacagatcagttcctttttttaacttttattttcagttcaggggtacaagtgcaggctgttttacaggtaaacttgtgttgtgggagtttggtacacagattatttaatcacccaggttttaagcctattacccattagttattttccctgatcctcttcttcctcccaccctccaccttctgaaaggccccagtgcccagtgtgtgctattcccctctatgtctctatgcattctcatcatttagctcccacatataagtgagaacatgcagtatttggttttctgtttctgcattagtttgctaaggataatggcttccagctccacccatgtccctgcaaaggacatgatcacattcctttttctggctgcatagtgttccatggtatacatgtatcacgttttctttatccagtctataattggtgggcatttaagttgattccatgtctttgctattgtgaatagtgctgcaatgaacatatgtgtgcctatgtctttataatagaataatttatattcctttgggtagaatggtatttctctctttaggtcttcaagaaatcaccacactgtcttccacaatggctgacctaatttgcacttccaccaagagtatataagtattcctttttctccacaactttgccagcatctgttattttttgactttttaataatagccattctgactggtgtgaaatggcatctcactgtggtttagatttgcatttctctaatcatcagtgatgttgagcattttttcatgtttgttggctgcatgtatgccttcttttgagaagtgtctgtttatatgctttgcccactttttaatggggttgttttgttttttcttgtaaatttgtttaaattccttatagatattagacttttgtcagatgcatagtttgcaaaaaattttcccccattctgtaggttgtttactctgtcgatagtttcttttgctgtgcagagctctttagtcgaattagatcccatttgacaatttttgcttgtgttgcacttgcttttggcatctttgtcatgaaatcgttgcccatacctatgtcctaaatggtattgcctaggttctgttacggtttttacagttttggatcttacatttaagtctttaatccattttgaggtgattttggtatatggtgtaagaaaggggttccattttaatcttctgcatatggctagccagttatcccagcacaatttatgaatagggaatcctttccccattgcttgtttttgtcaggtttgtcaatgatcatagtttgtgtggtcttatttctgggttctctattctgttccattggtctatatgtctatttttgtaccagtaccatgctgtttcggttactgtagccttgaagtatagttgaaatcaggtagcatacactggacgtggtggctcatgcctgtaatcccagcactttgggaggcagaggcaggtggatcatgaggtcaggagttcgagaccagtctggtcaacaaggtgaaaccttgtctctactaaagatacaaaaaattagctgagcgtggtggcatgtgcctgtaatcccagctactcaggaggctgagaaaaggagaatcacttgaacccaggaggcagaggttgcagtgagccaggattacacaattgcaccccagcctggatgacagggtgagactccatctcaaaaaaaaaaaaaaaaaatcaggtagtgtaatgcctccagctttgttccttttgtttaggattgccttggctattcaggctcttttttggttccaggtgaattttaaaatagttttttctagttctgtgaagaatgtcagtggtagtggccgggcacggtggctcacgcctgtaatcccagcactttgggaggctgaggcagtggatcatgaggtcaggagatggagaccatcctggctaacggggtgaaaccccatctttactaaaagtacaaaaacttagcccagcatggtggcgggtacctatagtcccagctactcgggaggctgaggtaggagaataaaaaaaacctcctgaattcgttgatcttttgagtggtttttcatgtctctgtctccttcaggagacaatacctttgttaattttttattattacgtcttgtcttctgctagcttagggatatgtttgctctttgttctcaagttcttttggttgtgattttaggttgttaacttgaaatctttctaactttttgatgtgggcatttaatgctataaatttctctcttaacactgccttagctctttcccagagattctggtacattgtatctttgttctaattatcttcaaagaacttcttgatttgtgccttaatttcattatttacccaaaagtcgttcaagaacaggttattcaatttctatgtaattgtatggtttgagtgaatttcttagtctttattttgaatttgattgcactgtggtccaagtgactatttgttatgatttcagttcttttgcatttgctgaggagtgttttacttccaattgtgtgattgattttagagtaagtgccatgtgacaatgcaaaaaatgtatattccattgctttggggtgaagacttctgtagatatttatcaggtccatttgatgcagggctgagttcaggtcctgaatatcttcgttaattttctgtcttgataatctgtctaatattgtcagtggagtattaaaatctcttactattattgtgtgagattctaagtctctttgaaggtctctaggaacctactttatgaatgtgggtgttcctgtataggatgcatatatatttcagatagttagatctccttgttgaattgaaccctttaccattatgtaatgcccttctttgtcttttttttatcttttttggtttaacacctgttttgtcagaaacaagagttgcaaccctggctttttttctgctttccatttgcttggtaaattttcttccatctttttgttttgagcctatgtgtgtcattgcatgtgaaatgggtgtattgaagacagcatactgataggtcttggttctttatccagcttgccactctatgtcttttaaatggggcatttagcccatttacatttaaggttagtattgatatgtatggatttgatcctatcatcatgatgttagctacttactttgcagactttcttatgtggttgctttattgtgtcactagtctgtgtacttcattgtgtttttgtagtggctggtaactgtctttccatgtttagtgcttccttcaggagttcttgtaatgcaggtctggtagtaatgaattcccttggcatttgtttgcctgaaaagaatctgatttctcctttgcttatgaagcttagtttgtttggatatgaaattctgggttggaaactcttttctttgagaatgttgaatactggacctcagtctcttctggcttgtaggatttccactgagacatccactgttagtctgatgggcttccctttgtaggtgatctggcctttctctctggatacccttgacattttttctttcattttaaccttggagcatctgatgattatttgtcttggggatgatcttctcatggagcagcttactggggtcctctgcatttctgaatttgaatgttggcctgtctagctaggttggggaagttctcatggatcatatcctgaaatgttttccaaattggttccattctccccatctctttcaggtacaccaatcattcatacatttgtttctttacataatcccatgtttttcagatgttttgttcattccttttcattcttttttctctagtcttgtcttcttgtcttatttcagaaagccagtcttcaagcactgagattcttttctccacttgctctattctgctattaatacttgtgagtgcaccacgaaattcttataaagtgttttttacctctatcaggttggttatcagattcttttgctgattccttctcatctctatgggcttatctaccctcaatctttgaggttgctgacctttgagtggattttttttctcttcttttgttttctcttttaacagtctggccacttttctatagggctgtacagtttgctaggggcctgctacagtccctagttgcctcagattttccagtacctgaaggtatcaccagtgaaggctatgtaacagcaaagatggcagtctgccccttcctctgggaggtctgttccaggaaagtacagacttgttgccagcctgaacacacctgtaggaggtgctggagaccctggttgggaggtcttgcccagtcaggaggaacaggatcaggatctagaggtttactaaaggaggaatgctcccacaggagacacagaaatgaaccattgagctggaagctaagacagctacccaggcccttcagagcctcatgattctgaatcaacaaaggaaggagttactgcactgtctggggtgattggtcctggccatcagggggaattggtctactactacacaatggcagtaaataggactgtgtctggaacacaggagatcttttagagcatctcttagtaatactctgccgtataattaaagtcaataaaaacggcaacaacccaagtcaggtaggactagtaatgacacatacccttcaggaatgaaggtttgagtcacccaagcaggaaaagaaacctgatcagctgacggacttgctgaaagaaatgggaatatgggatggttagtgaaaaacaacaaaaaaagtatttataaatactagttgtggccggcacagtggcttgtgcctgtaatcccagcactttgggaggcggaggcaggtggattgcttaagtctaggaatgtgagaccaacctgagcaaaatgctgaaactccatctctactgaaaataaatacaaaaaattagtcagacttggtggtgtgcttgtagtcccagctacttaggaggttgaggtaggaggatcacttgtgcccaggaggtggaggttgtagtgagccaaggtaatgccactgcactccagcctgggcaacagaacaagaccctgtctcaaataaataagtaantaantaagtaaataaataaataaataaccagctgtaaccatgtgagcacttacagaaataaagacagtaaatgttatcagcattacttctttatgtttttataatgatatttatgtctgtgtgtatgtatccttacatgtattgagcaaatacctttcttttcatcttttcctgatgatctgtaataaaacataagatgtgctaacaataattcccttttattaataaaagttaacttttattacgcatggtatttaggttacagtatatcaaagagaagagtgcatatcatgcatctttttctgtggaaagggttagtgtgtttctagttggatgcaagttaattgtatcatctttggcaaaagtaagactttataattgtttttatttggagacgaagtaaggtttaaggaaaatgttaatgggtgccaagttcacaagcatggactgggatggtgggtcttatgtgtcaattttactaaaccatagcacctagttatttaatcaaacactaaatttatatgttgatatgatggtgttttgcagttgtgattaaaatctgatatcagttgatttcatgaaaaggaaattaccctaaaacatgtgggtgggactttttgaatcacttgaaggcctttgggcgaaatgtgagtcttcccagagaagaaaaaattctgcctcaagatgacagtagcaactacactttaggtttccaatctgccagcctggtctacaaattttagattgaaggctgcaacattatgactttccagccttccagcctgccgaggtgtaacttgtgacatcaataacgtaaaatgaggggtgggaaaagatgtaaagaagcagagtttcatattgatttcagttcaagattgttataactgtattgttatatataataccacagtaatcacaaagaaatatacataaaatatacacaaaagaaaatgagaaggcagttaaaacatatcactagaaaaaaatcgactaaatgaaaaatgactaaaaacaaaagaaggttggaatgaaggagatgaaggacaaaacagctataagacatgcataaaaccaacatcacaattacagaagtaaatccttccttatcagtaattactttaaatgtaaatgtaaatggattaaacttcccaatcagaattcagagattatcacaatggacagaataaaaaggatctggctgggcacggtggctcacacttttaatctcagcactttgggagacggtggctggcagatcacaaggtcaggagttcgagaccagcctggccaacatgatgaaaccccgtctctactaaaaatagaaaaaaattagccgggtgtagtggcaggtgcctgtaatctcagttacaagggaggctaaggcaggagaattgctggaaccggggaggcagaggtttcagtgagctaagatcgtaccactgcactccagcctgggcaacagagtgagactccgtctcaaaaaaaaaaaaaaaagattctcactttggatctaacgacacacagaagttgaaattggaaagacagagaaggatattcacaaaaatagtaactaagaatttttaatgatctcttatagggttctatgtgttggctgggctcagatgatggcctgggagtgttacatggtccagggattgaagtggaaggaaccagacgcaggcaggatggttaacagcagtgcccagactgacacagccacacttctgccatattttattgggtaacgcagatatcagatctgcttatgtttaaggagtggagagaacttacctcttgatgagggagtggtgggtcaaactaaagaaaagcaagtgcgatggaagatatttagatatttttgcagccctctttggaaaatacatgcaattcaaaacatcctatctgccagaaaggaagaacaccattacgccttacccctccagtttaagccctgtgattctatagtatcctggggaaggatcatgtcttaggaagcaggtggggagtcgaagctctaaataaagccccatctacacccacaatttcactttattttgatgaaattttgtgtcatttttgatgaaatgggggctcacaaatctctcttcagggatctctccagtggcggggacgatatgctcccttctccacagttctactttatagataagtggaactagatctcagtttcagttatttaaaacaccagagacttggatgcatgcatgtcacaaaaaggtgaaagaatgctctctgtcactttcccttctctcactcactgcgccctgaacaagaacctgctctaccctgaccttttgaatgaggaggagggtcgcactcctgcctaaccttcaactttccagagataaacctcctgcccaaacagcatggttcattataaagaccaaaccctacaccaagagctctccccttatccaccatggcagaaactgaatcctatggaaaggcccagaaaattctggctcgaaatgaccagcgtcatgtcccttccaccaaaataaagccaacatattcatcacaacatggggtattgataaggatatcaaaatgattaccaaaaaaatttgacaaatatgttctttttattacatcaacttttgtttccctccaaatataaagaaaataagcctgcacagcaggagaagttgttcccctgggagagcatcctctttccagctgaaaatctggtactggaatcactagaatggaacaagggtttctggtatgacacagcaccaggtcttggggcagccagagttagggagggctcaccctctgggtgacaccaacctttggttctccatttttttgttctcattgcaagaacaaaatttcagagtatcagagccaaagaaatttactctattttattctttagaaaatgcaatctcaaggataatcagactgttatgggctaaattgtatccccccaaaattggcatgttgatgtcctaacccccagtacctgagagagtgactgcatttggagaggttttaaggaggtcattaaggtaaaatgaggtcatgtgagtgtgccctaattcaatgtgactagtgtcttcataaaaagagaagattagacacagacagatacggaggaatgaccatgtgaagacagagagagaacacatctacaagccaaggaaagaaccttagaagaaatcagcactgccaacaccatgatcttgaacttccagcctccagaactgtgagacaatgaatttctgttgtttaagtcacccagtgtgtggtactttgttatggcagctctagcaaattaaaacaaacatattatattgcccaatggagagttttacctgactccactctggctccaatttgattaaggtctatatgctctgggacctctccagctcctcatgagaaatgacaaagaatgccatgtccagctcctggggtccctgatggcagtgagaggcagctcctaatcgagggagctttggggtgacgaaggcctcatagacttcatcactgttgggccacttggcttcagtcccctgaccacaatggatccatgggcctggattcacctctgacattcgtgtcttttttttccacaagtggtctctggtagagatggagggaaaggacacagggattcaaaatcctgactatcctagtgtcctcccctcacttccactcagagacaacacggccatttctctaaactcctgaggacgtggccaacataagtaatggagcacggggaggtgccttgaacagagctgcatggaagggtggctggtggcctctagaccctttgtctatacgtgctgggcttctttagtatgaaatgtcccagagacagccacattctatgcttggatcctcaaaccaaaatgtgtgagggtcttagccctggccaactgagcactgaattcaagagagaccaggaaaccttctgaatattgggacactttattcctgaatcctgaatcctggaagttggcttctggccagggaggcaggtgtcattatatctgggctgcctctgtgaggcctcttttccctctgcctttcacatctttctacccctgttaaaatcccagaacagatagaaacattctgccttcttagatgcctcttgctacaaatttcaatgccatttgattgaattaataaatgaaagcaactttaatacaagatgaggtttaaagaatggtatttgacatgggtttagaatgaataaatggatcaagtaatgaattttgagaaaattcatgtaaaatttatttttaaccattttattaacttgactgtggaattaataaatgattccaaattaatacaaaaaagtgcaatcacaaatgtatgaatgcccagaagtgtgaatgaggggcaatgcacacaagaaaaaaggtgtgcacaggaagaaattcttcattccacgtttttactgatccctctggtcaagatgggctaaaagtccaaaaatctgctttcctcacatggtcaaatgattcagcataaaatacaactgactgaacacaattatcaatgctttaaatatattagaaaaaatcaataaggaaaaataatcctaccatcactaagattttaagttataataaaataaagcctagaaacgtatcgtttgttcccaattccaatgattataggattgcagtgtttggctttcaaaagaccatttaaaacaactcagggaagttttcttaacaaactctatagcaggaccacagctaaacctatgagtagtagtagaagtaaacaatgacactgtctttaaaatttcattttgaaaaacatgtttgaaagtggctgactggctcctaatttaaccactaccatttgaagtcatatgttagattggcaaagtcgtcgtaatttttatgtattttatttttgtaatgcaagtgcttcactctgctatctgataaaagaaaataccaagccaggcatggtgatgcatgccttgtagtcccagctactcaagagcctgaggcaggaggttccttcagcccaggagttcaaggctgcagcaagctatgatggtgccactgcactccagcctgggcaacagataatgcaattttaatgtaaaattttagtgttaggagttcaaccttgaactgattctgtgagatcctatttgagagaagctgttgtgtacaatgaagtttggggaatatcaacaagaagtaaatgcctaccttgtttctaatttacaaactaaaaggaaattccataaacttttaatgctaagctaaatcattgtagaatgaatgacaaaaaaattatcctacaaaacataactagttgctagatatatttgaacattaattagaaaaaagatgaatgaaagccatgttttaggaataaaaactacaaaagaactgtaaaagcttatcatgcccttggctggagttacgcaaagttactactcacccgtaaattgggattagcatggtggctacttcttaaagttgatacaaggattaaatgtgttttaatgcatcaaaagcatctagaaaagtgcctgagacatagtaagtgctaaagtctctctaagtaaataaaaaatatataaaagtgatgctaaattcacgattctgatcctctgtgcacacccaatgtgtttaaaattctgttcatcttcccttccacattcacagtgaaatgtttctgattaatttaatgggctgtgtgaggtgttcattatcttttacattaaacttggtacattgtacaacactcttatattgtgataatttaccaagctaacacaggcttaccttgtttttaatccttttcacacacagcatagaattatattgctatatttttaaatttcagatgatttgaaaaatatttacactatagtagccaaacagtaataattataagcaacttggataaaacatttgaaattagagaactatatttagaataaatttctcaccttacagaaaaatttcccattgaaaacccctttatttcgcatacaaataagaattcagcatgtttttctatacccagagagaacagagataaacagggtcctttcgtgtggtttgggtattttattagatgctgagttcaagaaagtctcacccctgaagaagctgaaattccatttttcgaagcttgctcctcaattccacatgcttatcatagatatcaagcagtgcccttgctgtctgcccatcttttctgaccctgggacaccaccctttatttactttccccacaactccttgaaggtcaagcccccttggctgatactctgggttatcacagtaaatgtgccctctatcttttgcaggtcactggaaaggagggttcctaaagcattcactcctgaccctgagggaggtgggtgcactcactccaggactcaggttcaccacagataagcaaaaaagtcctcacattcaaaattgtcctgggccacatgcagcccacgagccgcgggttggacaagcctgcatctagaacgtatgcaccataaccccagacatttttctgtttttactttaaggatgctttctaaatgcaaaagcagtcatatccctagcagacaacaaatgtcagttgaatgaatgatcactgtagagcacctccctattctaaagcccatatctttattaatgtagccacagggcaaatgctgttttgtggcagctaaagaacataagttcacgtggacatccatgccgccagtgcttttctcaccagggctgcttgtgtgtcctccctcctcccacaccaaccctcctgcacacttcagcacaaaactatatatttctcttcaggaacaataaccctagccttatgggtacaattttccaaccacatatgaatctaaattagactctgctttataaatctatgagtttggattggagccagcactaggattactacaactcagagcaggaagaagagtaggagggcagaagaggagttccaacagaaagttaaccatgatgagaaactatgggacccctcctctctgcaaatgtcacaatctggttcctttaaaaagattggagaaaaatcgaaaataccattaaggaaaaagccctgggaggaaggcaagacctggacaggtctgaaaatttgtctcttgataccacaaggagatttgtatgcagggaccaccctaggtgacatccaactccctgtgatcacaggtcttggtgggacaaggttctactgaagggccaaggacaatggagcagcaaagatgacccaggtgagctgtgaccacataaagcccatggtggccagaacagaaactgggcacagccccatctactctcctcccctgcaacaaatcagcacaagaaacacgtggactctggaaggttctcatgtcttccatttattttgtctctcaaattttaggaatcttctcctttaattaactcatcaacctctcatggcaagaatttgagaaagtaaatttatactcaggttctaattttaatagggaaggaagaagttacagctcagtgcaccatgaagttgagacagagatggagacacctcagccccacctctctggaacaggaaagatgattggggagggagcacaggtcagcgtgggaagagggtcatggtggacatgggggtggggtggtctccccacctcctcacattatgcctacacgaacacagacacatgcaggtgcctttgcagaaacaaagtcagggttcttcaagtcacaaagggaagggcaggaacaactcttgcctctcagtcccacacaaggcagctgtctcacactatagaaaaaaatattcatgaacaaattcatatccatcacagtgaggggtcacaccttaaacagcccatcgcatgctcaatacatccaatgcaaagaaaccccatagcacagctgtgtccactgttccgcccaacacccaacacacattaggtcctccaagctctcacctttacaagctgtgagagacacatcagagccctgggcactgtcactgcctggggtagaacaaaaacagaacctggtcagatcccacagaagatgtggctagaggaggaattgtggggtgggtgagctcccccatgggctcccaaacacaatatcccaaggacctcaggcatcagcctccttcatacttacttgcagcctgagtgtaactccctccttttctatctgtgagaagaaaatgtcctgtgagataccagaaaggagccagggccttaaggtcctagaggaacctcctagtcttggaccccagagaagtttccagaaatgtgtgactgcagacccagggcgggatcaggaaacatgaagaaagcaggtgtgggtcctggaccaatagccctcctgaggtctgtcctcagggaccttcccctgtgacttgtgactgctgggatcaggtcccatcaccgccgtaatcaaggtgataaatctgtccttcattttaacaggtgctttacaaaagagtaagtgctggcacacagggcccaggctgggtaggcccatgattgtggatggtgcttcccagtaatgagacagggcacatttctagctggggcttggaaccctcagtgagacaagaaatctcagaccccacccttcaccccttctccacctgagctcttcctcctccacatcacggcagcgaccacagctccagtgatcacagctccaaggagaaccaggccagcaatgatgcccacgatggggatggtgggctgggaagacagctctgggaaaagaggggaaggtgaggggccctgaccctgctaaaggtctccagagaggctcctgctttccctaagagacatgacacccccatctccctccttaccccatctcagggtgaggggcttgggcagaccctcatgctgcacatggcaggtgtatctctgctcctctccagaaggcaccaccacagccgcccacttctggaaggttccatcccctgcaggcctggtctccacgagctccgtgtcctgggtctggtcctccccatcccgctgccaggtcagtgtgatctccgcagggtagaagcccagggcccagcacctcagggtggcctcatggtcagagatggggtggtgggtcatatgtgtcttgggggggtctgacgggaagagtcagaaaattcaggcattttgcatctgtcatgggacactccaccagcacgcatgtggccatcttgagaatggacaggacacccgggatggggaagagagcacagaacccagacaccagcctggacacaggcacctgggataatcttctattccctgagaagggaacagcgacttctggtcctgacctgagtggaggctgagggactcagaagtgctggactcagacccccacacacattgagtgtgaagcagagaacaaggcctgagaggaaaagtcacgggcccaaggctgctgccggtgtcaaagggaaccactcatcagtattcgagggatcgtcttcccgtcactccttcagagattttatcccttaattgtgtcagagagcagggcggaacctcagagtcactctctggtacaggatctggaaacccaggaggattcctctccctcaggaccagagggagggtgatattctagtgttggtcccaattgtctcccctccttgtgggaggccagcccgggagatctacaggcgatcaggaaggcgccccgtggcccctggtacccgtgcgctgcagcgtctccttcccgttctccaggtatctgcggagccactccacgcacgtgccctccaggtaggctctcaactgctccgcccaacgggccgcctcccacttgcgctgggtgatctgagccgccatgtccgccgcggtccaagagcgcaggtcctcgttcagggcgatgtaatccttgccgtcgtaggcgtgctgttcatacccgcggaggaagcgcccgtccgaccccacgtcgcagccatacattatctggatggtgtgagaacctggccccgaccccgcggtcagcccagtcccccgagccccgcccagccccgaccaacccgggggaatttttggcctaaactgaaaatgaaaccgggtaaaggcgcctgggcctctcccgggtcaagggtctcggggtcccgcggcttcggggtggatctcggacccggagactgtgggcgacctggcccgtccgtgggggatgaggggtcgtgacctgcgcccccggccggggtcactcaccggcctcgctctggttgtagtagccgcgcagggtccccaggtccactcggtcagtctgtgactgggccttcacattccgtgtctcctggtcccaatactcaggcctctcctgctctatccacggcgcccgcggctccatcctctggctcgcggcgtcgctgtcgaaccgcacgaactgcgtgtcgtccacgtagcccactgcgatgaagcggggctctccactgccgggccgggacacggatgtggagaaatacctcatggagtgggagcctgggggcgagcagtggctgagacctgaccgaccctcctcccggcgcggctcccccggtcctgcgcccccgccaggagggccccttgcttctccccgcagaggcggtttccctcccgaccccgcactcacccgcccaggtctgggtcagggccagggcccccgagagtagcaggaggagggttcggggcgccatgacggccatcctcggcgtctggggagaatctgagtcccggtgggtgcgtgcgggctttagaacagcgaccgcgacgacactgattggcttctctggaaacccgacacccaatgggagtgagaactgggtccgcgtcgtgagtatccaggatgaaggaccctacgtaggttgggagagggagaaaagaaactgcggagttggggaatccccaaggctgtgactccccaatccatacaccgccttcggggcctgagaccctgagagccacgcctggggccctgggacttcgccctgacccctctgctcctgtgcgaagcgctctgtctcaatgtctccctgagtcttggcccaagagctgtctgagaaacaagggagaaaccctcggttcacttttcatcccattatctctgtccctgaactggactccctgactcccactccttacctgtccacctggactcttctagaagaaaaatcaccccatggagtttggtgccagagagtaagcttgctctgggaatggaggtgtagatttttgttgttgttgtttttgtttttaatccggaaaagttgtgcctgagtgcatgagacagaatagagaccaatttgctctttgtttattaactacagtgggtagcagaatcttggtaacccctgaatgatcaggaatctaataggtaaaaaaatgtgactttggccccttaatgtataaatgtgtccaaaagcattacaacaggactcacaaagctactaagttgcactttcccagacaatgtatctgtgactcctgcttgtcttactttaaatttgccatcattccttagctctgagtttctgtgtgagtccacgacatctcctcaatacaaagtagccactgtgttcctatatgttgcaaccagaagccattacaggctttattcacctcaaagttgcaactgttcaaggcagtcacaatgcccctcactagtgctcatgcactgcctgtttttaggaagtatccacttctaagtgttgtgtatattttataggaacacttagtaatttttaaacctgattaacataaaaaatagtttttaggcagacccacatgaggtattaaaagacaactgccaaggacaccctgctaggctctgtagattgatgtattaaaatccataaaacaatgtctttaaacctaagaattctgctgctttcaaattctttcctctgctccttttcctcacctcctgcttctccagcccttccctccatccttctcatccttcaggccctcctctccccttagtccccaccacactgtcactcatgaattgtgacactagcactgtcccatgacctgctacgtaactgttcttttcacagtggccctgctcctgtgagtcggagtgtgtcatttctccacttaaaacactccagtgtctccacctcggtcttgtgaagcttctggagtgtcaggcacttgagcatatgaggggatacctcgttcattgtagggactaagttaatttttgttgacttaatgaatgaaatatgagtgtattaaattgcatcacagataattataaactgtaaaacactgaaaaagttcagaaagattttattttatgcaactagtgtgtatatcaattcatcaattcattcgcacactaccatgcctggcaaaacaacacacatttatctgcttatagttccttggtcagaaatctgggcaagatgtggatagaatctccattctgggcttccaaaagctgtgttttcattttgaatcctccttcgggtttatacagaggtggcagaatgcagtttcttgcagttgtaagactgaggtccctgttcctggctagctgtcaatgtagagaactgggaggggctcaattcccggtgctcaccagcgttctttcctacacagccccttaatttttctttttttttttttttttgagacggagtctggctctgtagcccaggctggggtacagtggcccaatgatctctgctcactacaagttctgcctcctgggttcacaccattctcctgtctcagtcccaagtagctgggactacaggcgcccaccactatgctctaattttttttttcttagtagagacagggtttcaccttgttagccaggattgtctcgacctcctgacctcatgatccgcccgcctcggcctcccaaagtgtcgggattacaggcgtgagccaccgcgcccggcccatagccccttccttttcaaagcccatggtggaggaaacccctcatgctgagtccctttcacactgtaagtctctatgctcatgaagaacccagtcctttcaagggctcacctgattaggacagtccaagcaggataaacccagcataaagtcaactaattgagacccttaattatatctgctaaatcccttcacagcagcacctacattagagttggttgaataactggaggaaggtgaatgaccaggagctgcttgttgaggccatcatagaatcagcctagcaagggttggatcttccttttgtgtttaattgggacacagttggaaagtgaagttcaagtaacgtgatcattgtgaatggtcataaaatacatcctcttcagccatggaaattgtccttactttttaaaactaagttacatgtttaatatcttataattaatttaggccaggtgtggtggctcacatctgtcatcctagcactgtggaaggcagaggaatggagatttgttgactccagaagttcaagatcagcctgggcaaaacctccatctatacaaaaaattagaaaattagccaggcatggtagttcatgatggtagtcgcagctactcaggaggctgaggtcagagagtcccttgaccccaggagttcgacactgcagtgcatggtgatcatgccactgcattccagcctgggtgacagagcaagaccctgtctcaaaaataataatgatgatgatgacacatttagagcaaatgcaatttgatgtgtaataatacatcctctcttgtgaaaatatattatttactgttgcataacaaattacgtaaaacttagctcgaaacaacaaatgtttctcatctcccacagtttccaatcgtcaggaatccaggagaggtttccctgagtgcttcctgctcagggcctctcacaaggttgcactccagttgtcagccaggggccgcagcatctgagggcttcgctggggctgggaattcacaagaaacatggatcagtcacatggctgttggaaaaggcctggttccttgttgtctgttcccagaaggcctcagttctcagtcatgtggaccttcctgcagggctgcttatggcacagcggctggcttccccaagagctcatgatcccagattcagagagagagaaggtggaagccgcagttagttttatgttctacacccagagtcacaaactgttatgttggcactattctataagttagaagttgtattagtccattctcacactgctataaagaaatacctgagactgggtaatttataaaggaaagaggtttaattgacccacagttctgcatgactgaggaggctgccccaggaaacttacagtcatggcagaagtggaagcaaacccgtccttcttcatgtggtgacaggagagagaaatgcacaatgaagcagggaaaagccccttataaaaccatcagatctcatgagtattccctcagcatcaggagaacagcatgggggcaccgtccccatgatccaatcaactcccatgaggtccctcccccaataggtagggattacaatttgcataacaactcaagatgacatttgggtgggaacacagaaccaggccatttcagaagtgcctcattaagtccaagccacactcaagagagggaattaagctgcacctccagaaaggaacagtatcaaaggatttgaatatatgttaaaagcaaaattataactattgtttcaggtttctgaaaatcaaagtcttcttgtatctaattatttttcattaactctttaagcttgtcttttaatttaattttttttttaagttccagggtacatgtgtaggatgtgcaggtttgttacataggtaaatgtgtgccatgttggtttgctgcacctgtcaactcatcacctaggtattaaccctggcatgcattagctatttttgctaatgatcctctcatcaccaccctcccccaacaggccccagtatgtgttgttcctctccctgtgtccatctgttctcattgctcagttcccagttatatatgagaacatgtggtgtttggttttctgttcctgtgtgagtttgctgaggataatggcttccaccttcatccatatccctgcaaaggacttgatctccttcctttttatggctgcataatattccatggtgtatatgtaccatattttctttatccagaccatcattgatgggcatttgggttgattccatgtctttgctattgtgaatagtgctgccatgaatgtaaacatgcatatgtctttataatagaatgatttatattcctttgggtatataccctgcaatgggattgctgggtcaaatggtatttctggttctaaatctttgaggaatcgccacactgtcactgtcttccacaatggattaaccatttacttttccaccaacagtgtaaaagcattcctatttctccgcaaccttgccagcatctgttgtttcttgactatttaataattgccattctgactggcataaaatggtatctcatttgtcgttttgatttgcatttctctaatgatcagtgatgggagctttctttttcatatttttgttggccacatgtatgtcttctttagaagtgtctgttcatgttctttgtccactttttaatgaagttttttttcatgtaaatttgcttaagttctttgtggattctgaatactagacttttgtcagatgaacagatggcaaaatttttctcccattctgtagcatgtctgttcactctgattgatgatagtttcttttgctgtgcagaaaaaaattgcctaatctgattgcttttcatttacaaaaatgagaatctcatacttcagttaacacatctagcttagcatgatgccaataattttggcttgatgctgtagcccgaaatggttagctcgaattgagagacaagttattttcaaaaactggctatccgatgggcattctataacacacttaacatcttttgttgtggtattgaatgagatgtgatagaagtgatttggacaccactatgattctaaatagcactgctattatgtgcttctgaattttttttctttttagcagtcctattctgtttccattcatatttgctattccatcactgggtattccttcctactcttttcatgtttcatttattttgatcagtgatgtgcatttccaattctgtaaaagtttaattcagttgtatgtgtggtaaaatgtaacatagaatctttgcaagatgaaattaccttgcacagcatattgaacattgtattattgaaaatctagagagatgccagtgagccaaggatcgaatgacctatatgtagccaaggccgttttcatggcatcctggtcctctctgtcacatagctccttacaattctctgtggttttcttccaatgtaagtagttcttgttaagaatctttgcaatgaactttgaaattcttttctttttcattgctataaaaaacaggtatattttaatttggaaaactctgtttaaggaattataatcaagtaactaccagggggcccagcactggtgtaaaacacaaaactgtaaaactaccctgggctgctacccttcgaaatggggtcttttcaaagttagtaacaatgaagctgtcattcacacgatgcatggctgaacaaaaagacagagacagcgggtgctgcattttatttttgtaatgttgccttgtccacaagacactatttacacttaaatcaattaaaattatattaattttaaaagtcagttttcccagtttctcatttgtactagccacatttcaaatactcagaattcatgggtggccggtgactgacgtaatgaacagcacagatataggcattccatcactacagaagggagctggacagcagtgtgccaatgttttaaggacagaatcctgagtagaattataggatgatgacaattccgagttcccattccaattcatggtctcttctctgatgagtggtgtagggggaaatccatctcttttgtgagtaaaggttaatttttccaactacagataaataattaacattactttttttaaaaccagttttacactgttaagttacaaccaaaagtgaagggcttgaattacatttacattttaacacatggttttaaagggaagatccgtgtgaaaaagaaacaaaactcagaaaaaaaacccagaagttttcataccaattcccatagcaataacaatcccttttctgtcgttagagttcaccctcattctgatttcatagtaatcctgttcatgtctttctttaaagctttaccccatgcatatatttctaaaacaacacaggttatctcaaactttatataaattttgaatttttcatacaaatggaatcatgctgtgtatattcttacaagacctcattttagatcaaatgtatgtttgctaagtttttataggtgtctgtattcatgtttattatttgtaacatttcattgaataaatacatcatatattaaattaacccttctctcgtttatggtcatttcatttgttgtcagattttttaaaattacaagcaatgctgttacaagtcttctagcactagtctgttggcacaatgggcacatatctgtcaaaattatacacctaggagtggagttactggtatatgtgtatgatatcccactcaaagagatgatgccacactgctagataaagtggctgtggcaagcaatgtgtggtagatcttgatggtctgtattctcatcagtgtttggaaaactcaataattttaatatagaaattctagtgcatttttcaaaatatctcattatgttttcctcattaaactttattgagatataaatcacatactactcacccatctaaagtatgcaattcaatggtttttcacatattcagagttttaaatctgcatatttaattttagaacattttcatcatcccaagataatcaacggctttataatatgtctctctatacaatgaagtaaatccataaattttgagttttcacttcaggagtgttttttctatcattggcaactttcctagcttgacaggcacctttccaagtacctttaacaattttatttttaatagagatttcattcagtaatcagatttggagagcactgacaaattttgccacattgagacttataattcatgaacatgtaatgtttacttattaaatactcttaaataaagtattgctggttttacttgtgcatcttttagtagatttattccaaagatcttgaaatatggtgctagtgttcacagtatctctttctttgttgaatttcagtgttggttgtggtatgtataaatataaataattttatatagttttaattcaatgactttgcttaaagcttttattaattcatgtagttgatatattctgttgcctgattgcactagctaggaaatccagtgtgattttgagtgcaatggagagggcaggtctgctggtatcttctctgtgcactcctgatccacactccatgtactctccctgctccgtgtcctggacactgtgctgcatggcctgctggaccacaaaaggcagccttgctttctggcttttatttgggatcggctaaatgggagcatcatcacggagagagtgatgaggagcactggttggagggatttctccccatagctttcagtattgacatagggagaaatgagatgtgattggcctggattcatcaatatgggatcttcacccagtatcttagattaaatgtgttattttgaacacctaagagtgatgttaatagtcagccaggttagctaattaaaacccgaattcaactgtggcctagcacagtgtttctcaaatttgactgagctttccatcacgtgggagattttaaatagccccaggtctagatcaacatgtaaaaccaatgaaatctaattcatcaaaagtaggatcctggaaacagtattgttcctaaaactctccaggtttgactccaatcaacagccaagttaaaaactagtagctcaaagcagtggttctcaatatttggtatgtatcaaaatcatctgggaattgtagcaaaactacacaggcccagatattattctaatttaacaggctagagccttgagtttattaaaacaaatcatctctccagattttgctatatgcaacagtttgtgaaaaccactagcctatatcaacagaatccctatcacaggcatactgcagaggaaagtgtctgaacacagctcagagagacggaaaagtgggaatgtatccattatgtgcaacctacttaacatctcctaaccacatctcccgggagggctgagaggatgccccccttaccaaggcattaaggtgccacatgacacatacaggtataaaaactgctaggtctgctgagcagtcaccaatcttgagtcctggatattcagaaattcttatccagtagctacaccaaagccagctgatatgtcagagcccctcaaataagcagaaggctggtgcccttgagaaaggaccctataattcactctgagataaattctttttacaggactttcccagaggaacatttagtcatttatcgctgtccactggggaatggaaaaccccaaataatctgggagctcaactttaatgtctttaatcctgtcaaagattaccctggggttaatactttgttgtaacctgcataaaaatctttccctacactaaagtaatgaagatattctactatattactgtaaagagatgagtacaatcacatttttttacaagagatgagtacaattgcatttttccagatagataaccaattgttgttaaattattttctaaatcgttttcctttacccactgatctgtaatgttgcctctgctctacatacgttcagggctttctattctgttcccaagttctagtttactaccctgtctcaacatctcgactctcttaattaaaataactctaacataagtcttaatatctgctaaggaaattctctctgcttattattcttatttagagtgtcatagctactttttctctttcatatttcaatataaggcttaaaattggcttactatgtgttgcaaaaatgcttgataaacatttgattgagatgtgtttgagttcttagagtactgtaggaaattaatgtcattattatattgaaccatccaagccaggatcctggtaaattgattcagttaattcactgtcttctttaattactgtcttctattattactcagtagggttttttaaatatcccaattgatatcttgtacagaatttgttaggtttagttttagaagtttatatgtacgcacttttgctgaatcaattttttaaaattttcattttatatctgtgcaaaacagctctataatttcttctacctaccaagcaaaaatatcttattaacttccggatcacgaggtcaggagatcaagaccatcctggctaacatggtgaaaccccatctctactaaaaatacaaaaaattagccgggcatggtggcgggcgcctgtaatcccagctactcgggaggctgaggcaggagaatggcctaagtaaatctgggaggcggagcttgcagtgagccgagatctcgccactgcactccagcctgggcgacagagcgagactccntctcaaaaaaaaaaaaaaaaaaaaaaaatatatatatatatatatataaatatatttatatatatatgtatcttattaacttcaagagtgttctaatcatttgtattttctgcattcagcatattaccatttgcaaattataagagttttgtgccttcttttcaaacctttttaagtatatttcttttcttctttatgacactgggaagatcctcaattacaatttttaataagagcagtgaaaactgctggaccactcaaacaaaaattgagctgccactcctgagaactgactgtgttgctcttatggatcagccctttgggacccatcctaatcccagggatttacagtgccacatatgcacccgcagcctttagatgtagactttatccacctagccctcaaaggtctcagacactgcccagagcagccaacatcacccaggacaaaagcaaccctgcagctcaacttgctcttctagatttctgccttctcctggttcctggcctggcaaatctttactgcttggttagccccttcatactttccatcagatttttcaaatatgtacccttttcccacttgtccagtagtactcaatgggagggatgatccaaattacataattaataattgctgaaaactaaagtacaatgtacaatttttatgtcaatgtcacatgccaagggagaaaatgcataggtatcagtagtgactacttaagcatgtgtaacttagaggtaataaaaacaaaaatgttaggtaatatctatctattccagattaaaatctacatcaataaattcaaacttctaggaaaaatatctgcatatgtaatatatgactgtgactccgatagacgaacgtgggaatatagtatgaagatacttctcatcgtcttcactctgtgaggcagtcagtgtgaaatgagaaccagggattcaaaatgtgttgcaagactatgaaaagcactagagtaagttctacacaaataaagcaatctcctcataaattacactgggcttatctgtgagatgcatgtccttatcaaaatggagttaattctaggtaggtgatcttgtttaaataaaatggactctgactcactagctactggtccaccatcggtttttccctttactaaaagggaggccccacactgagccagcatttcccaagaggaggtgtcttgctgacatcaacttccaacagggtaaattttcatgggaacccaaagagaagaggaagacctcaagaaaaagggcctaaagaaatgacaagaatgataacattttacacttttattgtgctatgtgtcaaatagtggtcttcgcactttaaaaatgccaattcattttattctgactacacatccatggcttgtgcctgttatttttccctttcatagttaatgccataggaatgaagatcacacaaaagtagggatggagccgggtttgcacccagacaattctgccccagggccatgctcacaccactgcacttttccagaatgtgagggtgggcggagagtccagctcagggagagagattggagtgagagaagaaggagagggggccgactggatcactgtgatggtctggacccacgattccaggagagaaagacaaggattatgtcactcagggaaaatatccaaagtctctggtttaaacttaagcatctccagctccaggacaggtggctggtgggaagacaaactaagccaaagccacagctctgaagatttccctctcctgagaattctgcaggggtttccctgacctcatggccacctctcacccttggctcttgtttttcccccaggactgataagggcgtccagatgggacgcctttatcagttggtctcgggttctagagaagccatcagagtcagtggaggatgagcttcataaagtgggagatctccaggatccttcctggactccaagatacccagagaagccggatcctgtgtcccggaacccccttcctgttcctttgtgggccctgaccttagggagaactgggctggtgagaagatcaggatgaactgggctggggaggcaagagggaaagggcggcttggagggcttagtagctcctctcgcggcgcctccgtcccgggctgggatctgcagactcctcaggtcacttctcccagagccgccgccccaagccaccctcccctggttcccgtccctctgtcccctccccagctccccccacacagtaagaagctcccaagtgagcggctccggtgccgggcgggacgtgggagggaggctgtgtccccgcggggaggcagggcgacggcgtccaggagagaggggagcggggcggtgtcccaggcaccctgcgcctctcccgcccgcaatgtgcgggttcccggagcccaggcccgcaccccgggtacttgaaggccgccgggctccgccttcaccacgtgggccctgagttcctccgcggcgccttcgtgggcgacacgcgattcctgagcttccacagccgcggcgagaatcagagcgtgaagcgcgggtggagaagatggcacagggctgtgggcgccctgaggaaagttctcgaagaccgcagggaatgtgttactgaggggcctgaggttcctgggcccgcaccaccaccagggtcagccggtgagtgacctcagactccgggcacaggtgtgccccgccccgaccccagagtgaccagcccggttctcccagggatgggggggcggggcctcagctgatccctctcaggtggggaagcgctggggccattttgctccaggtttctccttcaatctggatcatgactgacttgttctgtgaccaatgacagagggaggaaggagctggggtgggcggggcgacctccctcccctccctggccccgcctccggctcaccctggggcaggtccagggcaccgcacctgcagatgctgctgtctggctgggtggtggctccggacaggagcttcctcgaaggacactttaagtccgcctacgacaaggagatttcatgtccctgaacgaggacctgcagacctggacagcggctcaaagagcggcttggaggatccatcacacctgggagaggttctggaccgcggaggccacaagggaagccccgatgggtcactgcactaggtggctgctcagacacctggaggatggcaaggagatgatgctgccggcaggtactggggtcctccaggccccacgtcaggacctcttggagctggaattatgttccttggaggcatccatatttgaggaaagaggagaccgaacagtctccgtctcctcctcctcctccttctcctcctccgtcgccgcctcctcctcctcctccctactgcggggttggggattctggcccaggctcctgaggctctcccatgggtgggtggggagggaggacagagaagaaggagtgcagggaggtaaataggaaaggacagtagttacccttgtcaacagtgatgtacttgggtctagcttcaccgggaatctttttctttcctggccctctgtgcccaacaattacatataatctttaacccaaccagaagataaaacaccaggaaacggaatcggcagagacacagtagatgaaagtaccacgaaggactgctaggttctaatagaaagtagcagatgtgagtatcaaaagttataaacctttttgcaaatcagaaaagaaacagactttttttttttttttttttttttttttttttttttgagacggagtctctctctgtcacccaggctggagtgcagtggcgcgatctcagctcactgcaagctccgcctcccaggttcacgccattctcctgcctcagcctcccgagtagctgggactacaggcgcccgccactaagcccggctaattttttgtatttttagtagagacggggtttcactgtgttagccaggatggtctcgatctcctgacctcatgatccgcccgcctcggcctcccagagtgctgggattacaggcgtgagccaccgcgcctggccgaaaaagacatttaaagactgaaaagtggtatcaccaatgttagaaccaaaaaactggacctgagggtgcatcacccgagggggttaagttaaactctgtccctgttccttctagagccccgtggagattcctgctgtgcagcaaagcctgggaggggcccctggctgtggaaggcaggctgagacgtcataggtgagataggaggaaggggacctgctggactcccggggacctgctggactcccggaggcctggtacccagggaaggccagttcctagggctgcctctggacttcagaaccttgatgttaagtcatattttcccgctcctccatgagcttccaggagcagggccacatggggctcagaaagtcccacagtgcctaggaagtgggtctgtctcttgtgggacaaaaattctcaggtctttctctttcagattcttaaatttcttaaaagagattgaactggaattacccaggccccaaactgaatcaaggaattgggactgtgtaatgtgcgtgtgtctgtctgtgcacatgtggatgtataaacaggtatgggcatctatgtgtatgtgcgtgtttataagtgcgtatatgtatgtatctgtacatatttatgtatataagcaggtatgggtatgtatgtgtgtatgtgtgtgtgttactctgtgacttcctcatacttggcaccaccctggccttttcctggttgccccccacaccataggttgtaagggacagaacagcgctccacctgcagacatagaacgggggtcaggataagtgttggggagagctggtttcatcacacctggaataaggaaacaggacctaaatggatgggtcccactcttttacagaattgtaccttaaaaaaagtgtcagtcacgttcctaagggatttacatgaattaaatcatttaaacctcacaagagccctatgacctagagacagctatcagccccattttacagacaaggaaactgaggtcactaggaagtttagtaactgtcagtgggctgactcatgactctaaggagacagtctgactgctgagcccaagtaactgctctgtcatactcaccctcacacccacctactactggctccctgtgggtcacctcctcctgtgggctctgaccagcttccctttcttctccaggcacagagactggactgaagtctattgcatttgccacagtcccgactggggagtcatggggttgaagtgacccataggctgatggagtgtgcttgctggatgcgggggaaagggagatttatatctgccctggagggagtcaatggtgtttgcatcatggcatatgaggtgcactatctagaatgctgaagggtaaagaccagcaggggccatgcagagtctattcatcacggttttatttcacagcctgcatgggcctgagagtggtggggtgttcactcatctccatccaatccctactggttcctgacctctcttcttccaactggtaatacctgcactcaaggttgaaccatgaggaggagtctacccacaccccacacttcctcctctctgcctgccactccctagtggggcctcctggatggtgtgagaggagagtgacggagatagtaagagaacttgacattcacactggtaaagagaaagtgggatgtcaacctatgagaaagataagggctgccacagggaaggggatgagataagttctaactggccccaagggtcgcttaactcagtgaaagctgtgggaggtgttttcagctatccattgctatgtaagaaattgccacaaacttagcagcttacaacaacacacatgtattatcccacatttctatgggtcaggaatccagacacagctatgcagctttctcacatgattttcatcagagtgccatggctgggtcttatctggaggctgaactggggaaggatccacttccaagctcaagcaattgttggcaggattcattcctccatgcctgttagactgacagcctcagttccttactggctggtggccagaggctgacttcagttccttgccacatgggcctttctgcttcatcaaaacttgcaaacaaagaaggtagaagacagagtcatctagtaatacagaagtgcggctgtaatatgacctcattacggaagtgacatcatttttgtcactttctattggttagaagcaagtttaggccccatcggttctcagctggatgagatgccaagagacaggatcaccgggcatcatcttagggtctctatacacaggctggaccatgactgaacaagatcattacaaacccgggtcctggactgggccagatgatctgcagagctgagctgtgtttatgaaatcgaatccctggaaacaaaaatcaagagacgcccactggaaaggctggtcttgcattctgtgtgaaagctaagatggcgttctctccaaatgtttttctctattacttttcaaacctcctgcatctgtaaagaagcaacaccactaagcctatctggtttttttttcattcgagtgtcatactgttaggcaaatcttctgctttcactatttcttcaagcttgttttcatgcagaatcccacgaacttctggtaattacactttattgatttcttttccttcactcagtttattttttagtgattttatttctcttgtatggataaattagtccatttaattagggaatttgtgtcttctaaagaaatatttctactattttttggaccatatgggctaagatcctcttgccaagcctcagctcacttcccacatggacacgacaaaattactgggggtcctggaaagtcggggattggcatagttcttaagcaggatcagacatgtaagtcacaaagatatgaggcagatacctttgatttccttatgaccctgatgacagcatacctggcactcaatacctgtctgtagaggtgaactcaactaaactccctgatcactcaataccatgtgagcccaaggacctcttatgtcatgggccaaattcccttgttttatgccatttttccagatggggtgctgttcacatggggacgtttctccttttccacctcattttccatgtgtgaaacactgggtcttgattactcagatctctcagggtcctttctaccttttcctccatcctaacattctccctacagttcccgccagggtttccccgaacctcttcttctccatgtattcagtctagcatctctcagttcattcttcacacttcaccaaagtgatattctggaatgcagatctgatcaaggttcttcctgacttcagtgctgccagtgctgcacattgtcctcggataaaagccaaaggttctccccaaccccacaagccttagtcattgaatctgctcactgctccaagcttgctttttgcccctgtgctcctctttttctaatctctaccctgaactgctgacagttcccctagactccctgtgtgttctttcttctgagcctttgtttgtgatactgattccctaaaagatttctcctgccattcacctggattaactgtgttcctcatccagttctctgtttagacattgcatcctcttggaagcagttttcccgcactaattatagtagcctgcatttagctgtttttttagtccccttgtggaatctttgatgtttcactcattgagggctaggagaccctgacacatagtaactgcctaataaatgtttgctaaataaatgtggggttcaccatgagataccacgttatacatgcaagaatggcattaatttggctgggcgcggtggctcaagtctgtaatcccagcactttgggaggccaaagcaggcagatcacgagatcaggagatcgaaaccatcctggctaacacggtgaaaccctgtctctaccagaaatacaaaaaattatctgggcgtggtggcatgcacctgtaatcccagctactcgggacgctgaggcaggagaattgctggaacctgggaggtggaggttgcagtgaaccaagatcataccactgcactccagcctgggagacagagtgagattctgtctcaaaaaaaaaaatggcattaattaaaaagtcaaaaaacaatagatgttggcatggatggattacactgctggttgtgagaatgtaaattagtacaactactatggaaaacagtatggagattccttaaagaactaaaactagatctatcatttgatccagcaatcccactactgggtatctacccaaaggaaaataagtcattatatgaaaaagacacatgcacacgcatgtttatagcaacacaattcataattgcagggatgtggaaccagcctaagtgctcattgaccaaccagtggctaaagaaagtgtggtgtatgtacaccatggattactactcagccataaaaaggaatgaaataatgccttatgtagcaacttagatggagctggaggccattatcctaagtggagtaactcaggaatggaaaaaccaaatactgaatgttctcacttataaatgagagctaagctatgaggacacaaaaacatgcaaaatgacataatggactctaggggcttcttggtggaggttgggaatagggtgagggataaaagtcaacactgggtagagtacactgctcgggtgatggtgcaccaaaatctcagaattcactactaaagaactcacacacataaccaaaaactacctgtcccccaaaaactattgaaataaaaaaatttttttaaataaatgtggggttgatttgtaatttaaaatttattttgaatagctaatacaagaacaatatttaaaagattataataataaaaatgtgtatagtgtgcaacgcaaatttccttctccaccccatccacaattcttccaaattccttgcctggatgcaaccactgttaccagtttcagcaagaaattaagacagaaattaaagttctgctcctcataataatcacgatcaattatccctgcttatacagtaacccctccttttcccaacagcctcctagcatgaggaacccaaattgaacaggtaacaaacatacccctaaacgtaatgtaacttgtactctgttccatggtaggaacttccttctctgggatcccaaactcctagaccagctgagtacgttgaggggatgggaagcaccaacaccccacgcaggttaccggaagtgctggtgagtatagtcattccttcttccagcactggtggttagagacttactctacctgttggagacttggcaccatataacagctgctggcgtaaccttagtgtatgcttcatcctgaaggacattacccatcctgataggatgtgatcttcaagataaaacccagctgcaccctcaagagaccattccactgctctatcagctcgcagagtgtgtgatgcagtatggggtagaaccaggagatctcatggtcatgtgcccactaccagacatccttcgctataaagtaggtcccttagtctggggcaatgatatgtgggtcccctattagaagatcatacatatgtgagcccttagataatgtttctgagtgtggcactgtgggaagaaagaacaaactcataccgctaatacaaggcaacctaaatctgacagaacccctgcctttttcattgtgaaaaagtaagaatagaatcaatttgccatcaaattgctcattggcttccataagcgaaggtaatctgttggagtttatttatcgctggaaagtagattgtgacaataaccacgtcagccatgctgaaggcagcccaagccttgggacccacccacagtgcatctcttccaccatagataactgctttcaagagcctgtttcatgagcactgggatggttaaggacagaggctggctgatgtgtaccgaatgattcatcctggacacctggttgtttagactacttgtatagagggtaatttttgctgagtagtaacatctgatacaaaagatccaagcactttgtgtctactgtatgcctctttccagatattcctgttcctgatcttccaatcttgctccttccaggcccctgacctgctatttaagccacttgctgttgcctataagtctgtgcatattctcacctcaggccacttctgtctacacaaagtgggtgaatcagagccccacctgcctattggcaggaccctcttcaatgctgtctctactcattcctgagttgcccacactgcagacacactctactgctgcctcacaccaacacactaagctggcatgtttatgaagaagctcagaattttcctcttaagtcagctggtcatagaaactacccataaggtcattggtgagagtcgaggtcaaggcaaaggtccagcagtgcgaggtgacatggggtctgagcaaggtctttacgtaattaacaccagtactcagaacatacgtactttaacccaggggtcagccaatggtttcaataaaggcccagatggtttccctttgctgtgtgtgtgttcattcccctctccgaagctatgtgtaaccccatagagatcacaggggttcccatcaaccacagccagttacatccacaaaaaccttgatccttgttagcagatgggagagggtggcttggaggattgaaatccctacttcatgggactctggatacttacacagttgcttccttctgtgtgtctacatgaatctcagcactgtgtttgggacaccatctacactcttattcttcatgcaattctgtttgctcctcagatggccgcttttcctttgaagctcaacagtagccatcacgtacttgggctgagggttactgattaactctgtgggattctgggatcagttccttccctttcttagccatccctgtgctttacaaaacttagcttcacaattatactcatctctccctgtgtgcacctctaagaaattactgataaaacacgagtttttttgaaattacaaaaatatatgcaagaaaatgtttatggggccaattgatcagcacggaaaagcataaactttcctgttttctgcctctcattagaatttaatcccccaaaattgaactcattccaaacttctggcttaactaggagttgctgtagccagcctattctagacagcatcctgtaagcccctgggattggcagacacaagacactttttccattcatatatgtggttctgttctggccagatcagtggacttccagtgcccttcttgagtcatactgaggtaaattactcagaccacgaactcacatttttaaataaataaaatagcagcatagttaagaatttatttatcccattactgggtatatatgcaaaggattataaatcattctactataaagacacatgcacacatatgttcactgcagcactatttacaatagcaaagacttggaaccaatccaaatgcccatcaatgatagactggacaaagaaaatgtggcacatacacaccatggaatgttatgcagccataaaaaagaatgagttaatgtcctttgcagagacatggatgaagctggaaacaatcgttctcagcaaactaacacagaaacagaaaaccaaacaccgcatgttctcacttataagcaggagttgaacaatgagaacatgtggacacagggagggaaacatcacataccggggtctgtcaggggatgggggcaaggggtgggatagcattaggagaaatacccaatgcatgcggggcttaaaacctagatgacgggttgatggatgcagcaaaccaccatggcacatgtgtacctatgtaatgaacctgtacattctgcacatatatcccagaacttaaaagtataatttaaaaaaaagaatttatttcatttgtatagtccatgaaattttttggacattttttaaacctgcacttttcatagtttttgatacatccttgctccaataaataatttgctaaaattactaaatctcatagatataccaatggtttgcatcagctaatggcatatttgcttgtcaaactgaggtcagccagtcccccttcattcactgttagtaactcatgtgttgcattgttaaaaacccagtatcacctcatctcccctctagcatcttctctgcagaggagaagccaggcactatatctcccaggatccccttccctggatagttccagtttacattttccagtgagagagacttgcataagaaatggggccctggagagatggcgggacagacctgtacccatcagtggtggctgcaggtggaggagcaggcagatgtcaggttctcagtggcttccctgctaggctagagacccacctgctttgcttgtgcggactaagatgagtgataagagcttcctcataggttctggagaatatagcaatctcgcagcaggattctgagaacctcccatccatgcttcaagctgaagtcttcagtatatgcttccctgacctccctactgcagcttccaggagctccaatggtgactggtattattatagtatcccactgctgctataacaaactaccacaactatgtggcttaaaacaacaccaatgtattctcttatagttctggaggtaagaagtcaaaaatgggtcactatgggctaaaattaacatgtcgtcagagctgcatttcttctgaggctctaaaggaaaatctgtttcctcgccctttctagcttcttggggctgcccgcattctttggcttgtggtctcttcttccatcttccaagtcagcagtcacatcactttgacctctgattctgttatcacagctctaactctgatgctgctttctactcctttcacttataaggactattgtgattacatcgaacctgcccagataatccaggataatcttcccatctcaagagcctcagcttaatcacatctgaaaatttccttttgccctattaggagacatttttataagttccagggattaggacacagacatatttaggagaccgttattctgcctgtcacatgacttcaccaatatttgctacccgtggtttccaaacattagtgtatgctcttatttctatttattcctgaaataccagagtgagtctgtttttctgaaaacagctcactaatgcccagaggagctcaaaaattttctgtctgttgtctcaaagtctccaaattgtactgattcttcaatgtgcatggctgtaaattaggcaataatcttgctggggtgttggaaggagaagaaagggaatgattagacagcccttctgattcgggacagaatctgattcgggacactgtccctgatggctggccatcttgggctgtaatccagtttccctcccacgtagacccggtcatttctgactccccaagaggttttcacagtattcatgtccacttccttcatcagtcatcttcttgtcttatatctccatgtttacatttttcttctcttctaccactgcagttctgccttgattcttcagccattgccttccttttgcatgcatgtgcttcagccagttctctaggaagacattccagacagctcattatcacttgttactatgtttatttgactgcagagctttcaagatttgtaactttctattttttaaattttagtgtaaaaataattcttgctcacagtaggaaatgaaacaatcaaaatatgcataaatatacttttaaatctctcctttcactaaatccaaacccacttaactaaggtgattactattgtatcagcctactctctgcatgtctactttcttcacttgcacgcaaaccatagtgagtgtttttatctggtagcttttgttgagctgtaatgactataagttttatatgtaatttaatcattttcagggcaacttgagatataatttaaatagtataaaatccgcacattcaaaatatacaattcagtggcttttagaatattcacagttgtgcctccattaccacaatcaattacagatccttttatctcctcccagccctgcagaaaaatccatatccaaaccccagccctaagaaactaccaacctatgttttaactctacagatttacctattctgaacattttatgtaatgaaattatacaacatgtggctttttgtgtctgtcttcttttacttaccagaacattttcaaagtttgtgtgggtcgtagcttgtatcagtacttcacatctttttcatcgctgaattacattccattgtatggatatatcacaatttatttattagttggtgacattttggtccttctcatttggattgtgattaataatgctgctataaacatggcatataggtttttatgtgaactcatgttttcatttctcttgggtatactgtatcataggaggggattgctgggtcatgtaataacttaatgttcacaaatttgagctactgctagcctgctttctaaaggggctatgtctcttaaatccccaccagcaaagtgtgagtattctagtatctccacattctcatcaacactaattattatctttttaattatagccattctagtgggtgggaaatagtatctcatttttgggttgatgtgcatttccctgatggttaatgatgttgaacatgtttctatgtgcttatgggccttttgtatattatttgtcttattattattattattattattattattatttattgtttattattatcttattattaatattattatattgagttacaaccattctttatatattctagatacaagtcctttccttgccagatataagatttgcaaaacttttctcccattcaatgagtttcttttcactttcttgatggtttccttagactcacacatttttaattttgatgatgttcaatttatcttttatttcctctttttgcttgtgcctttggtatcatttaagatcaggtatttttacctaatccaaagtcatgaagatttatgcttatgttttcttctaagagttctctaatattaagtctcacatttaggtctttgatacactgaggtaatttttatatgtggtgtgaagtagggatccagctttattcttttgcatagggatagactgttgtttctgcactgtttgatgaaaagataatttcttcccctcactgaattatattagcactgttttataaaaccaattaaccatataagtgaagagtattgctgggccctcaattttcttctgacttcattctttttaatgctgttgtaagttggatttttaaataacatttgtattaatgcattgatactctatagaagcacactcaatttttgtattttgatcttgtagcctaccaatgtgctaaactaatttagtattagtggtttagtagttgctaagcttgtttttagtgaattctttcgtatttctatataagattttatcatctgcaattgagatagttttacctcttgctttttaatctagatgacttttatttcatcttattgctcattgtagcttttgaaaaattacattgtattcacagtgccctaccatttgatatttttatttctattatatcatgaacatcctttgaatttcacagaaaaaatggtttaactcatcattttaatgctgttccatagtatgttatatgcacctactcccctggggtaaatatttaggtggtttcatttatttgtcacccactctacagatgtctatatgcacatattctcccagccatcccagagttttctgatttcccataacgtcagtgattaaactcctgatctgtctgactagctagacatgctgactgactgacagttccattgctactttgcataatttaagatcaacagctacatttggatctcagatcatctaggcccagctgcagttagtctttcctagtctcagagattccctctctgcctgttaatttctctatccctgaaactgcccttgtactccagcccaagcagcttcctagcccagtagtcctggatctgccacttgctaactagaaggtgtcatctgcagaaggtaagtaaggcatttatctccattcactttatatttagttatcaaatctccatgtagctcaggtagatcgagctgaaaacattcttgtgtttagtaatatagccattgagcacaccatcattgctcttttcataccttagtcctactcaaagattaaagtgacagcttcccagttatgataaaaaagtataaaacattctgagggacctgtaaactataaagctaaaatatcagcttctgcatgcaacatatatgtcaaattctatgtccaatactcttctcatttttagaattaattctgcaaacttgaagattgagctttttatgaactgattggcatctgctatgctcaccagattattcccacttgactgagaggatggtggtagcaagagctccttcctttcagaggcttttaaaaatacttattgggaagaatgtaccacataaggcatttaactaatgaaaccacagatataaccactggcaaaaaaatcaaacaaacatttctttaactgccctgtacttggtacattctcttcctcctgctagggaagtatctcctctcccttcacttcacagactggaattttccaaattcctagtctcaacaagacaaactgtattttgaaatcactgttttcccagaatacccggagtgttaccatgactccagaagatgccttcaaccaagactcataatctgactgtaagaaaccttcagataaggtattgggaacttctgtaaaattgtagctgggctgtgattagagaattttcacaatatcaacgaatcttacaaaagagctgctgatccaacaggtcctcagagatcctgtgtgggaaccagaaggtattctgtgaaaaacgacaataagaataaccataataacaataatggctgcatttaaaaggctcttgctatatttcaggcactggtttttgtacctacatggattattttgttcaatatttatcacaaccatatgagaaggatgttattatccacaataaataaatgaaatctgtaaactgtaacttgttcaagaaaattaggtacagagatgttaagttacttgcccaaagttccataattttgaagatatgaggctagaattcagttccaggccattttacactagatctactaatttagccactgtatgattgtaccaagactgacctaaagctacctccccttccccaacaggttcttttatgacagtgagtataaaatacagggctaatgacgtattgggtgcagagaggggttggaaagaatgtggtatttcagactatgcttactatgcttaaaaatcacaattccgtggtgactagagtttatcttaaactttcctccctggcattccattgcatccagttgagagagcactctagctccctacatcccagaacatgtagatctgtccgtggttctgaaagacacactggcaggtcccgccaactacatgaatggcattcatattgtggaaaactgcccactagtaaaaatgtacagttgaatttgtacattcgttacttacaaggttggagaacgtgaagtactggacaatccaacaatagcttggaaagataccataaaaagacccgcagaatattcttttatatgcattattctgaaatgtacaaaacaaaaccagaatgggacacattatgagtgagaagtttatcttgaaattttaatcagaatatgaagaaaagaaatccaacattagacctggaaaaagataaaataatttgtatttcatttaacaatatttatagattctcaatatatacactatccttttaaacctcaatttatatttaaccatccatttacctttattttttgtttgtttttttctttcacctcatactttctacctgggatatctttttgtgactaaagtacattctttagaacttcttgtactttactaggtgtgtttgtgttaaattgtttgtcttcaaaattcattgagatttaaattttattattatattttcatgcttaagtgttatgtgtttgtctcttcaaatttttctgaagattatttatagttttatagtcactaaatgtatttaacaatctgttctttaatcatagcccatttagcaattttatatcctatacaattattctgatttctcagttatatgattgtatgattctgctgtctgttgtattttcttgttctttcacggtgctttgtctcttatgaacattgttctctgttgttgttcttaactgttaagtgctccttttgctggtgtaatgattttttggaattttcagagtgccaatgacagtagaaattttccagagaagttttgcttgattttccaaggttccttggagtctgcactactttaaaataaattcatggcttgatgattttcagaccacccagggagtgtgaactcgggctgcaaatccacttctctttaatctcagcagaaggttttgcccctctccactcagcaccaaggtgactttctagggagaccttcaagcaaggatgttttccttatactgaggagctggctttgggatcctggtgaaatgaggagaacactctattaaatttctcactctgagtgctacctgaactctgtcttctattccaaatgccttaggaggccatgaaaactaaaccttaattatcctacattaagtaaataagctcagcaaaaaaagtaaatgcacaattccgtgatttaggatttgacaatttttattaaagtttaagcctctcaatgcttctacattgtgttttattttttcaccatccatttttgttagcatgagggtaggtatgtttacctaaatgtgccatgtgagtggttcaaatctcctccccaacgctccttatcacaaaccttaccatcttattcctttaaagtcactgactatctaagcaaactactagctactgctgtgcattgattaggacccaacagtaagacgtttctccttttggtaaactgcacttccagatggtctaattgaacatcttctcactggaagtatttttatttccattactcttaatgatgattcctgaaagagactctaatgccctgacagttaactctggggtatccaagcatactcaaatttttttccttctacagtcaattgtccttagggaactcctcaagacctcaataatgagacaagggagagtggactctccagtggtaataagaacattaggagtatagtacaaattattaggcaacttacctggccttatggaacctgctaaagtctgatgctgtgtataccatttccccttgcagactgagggctggtgagcatgagtgacctgataaaactcaggtcataatgggcatctcaagtcacttggtcattttctgtatcatgatcatagaattcagaatctaccagggcccagtagtaagccagaagctattactcaaattcagaaaaagaatggaactttttccccaaaccctgaaacttgggcaatgagtcctatgactgccacacacaaacacacacacacacacacacacacacacacacacacacacatacactcaacaatatccaaataagcatttagggccaaatggcagatatgcttgacttttgcctggatcaaaatgacatttccatttattatggaatgtttctgtacttgtactagctcataggattttggattagagaaatacccttgcaggatatgaagtcatactttttcattggtgaaatcatctgttctcattagagtttaataatatcatgcagttatttctcaaagagcttgtactccttagatttgctggaattcctcaagttaaaaagaccagggcctcaataatgggaggtctcattgccaatagctacaactggctgtagaatttattaaaaacatgtgagtgcattgtttggggaaagaatcaagaacaaaagcagtgcttgttttgtacttcactgaactacttccatggcctccattcaactttagctgcagttttggtgacttgattcacagactgtcggaaagttatgagctatggaatgtgctctctccaatactaaagaggccagtcatatatatgtagggtgtccttctttatcattgtaggttgtatgaagtcagtgattcccatagaaccttggggtcacagatgccaggcctaatcttcttttattattcattcaatctttattttttcaaacaatggtctcactctcttgcccaggctggagtccaatggtgcgaacacaactcactgcagcctcgaccgccccaggcttaggtgatcctcccacctcagccttccaagtagccgggactacagctgtgtgccaccgcacctggctaatttttgtgttttttgtagagatgggtttttgtcatgttacccaggcttgtcttgaactcctgggctcaagcaatccacccaccttggcctcccaaagttgtgggattacagacatgagccactgcacccagctctaatcttattttaattttagaaatctaacttgacagtttctggtgcctggatccatatttgtctcagaagattgaattgtggactgtgaccttctctcccaaaagtctctttcagggtagaacttctggttatggcaacatgaagagatagaccagcaagttttcctccaacagcaacatgaaaactgtactaaatactaaaaacaaacatttgaagtcactggaaaattaacaaaggcaagcaatatgtgataagagctttcttgtttaagactccttctgtcttaggtaaggactgcaagttggtggcctttccttcttacctatgagtgctcttcagcctcccagcagataactgcagccctagtggctcaatatgggcagtttatagagttcagagcccaaatcagctagaaatgtaaatgagaatgttgaaagagagctgcagaagagttgagatcaacaatgtgaatgcaaattatgctcatccctagttgaacactaaactctgcatgggtgtgggccacttgggaatctggtgaaaaaccagaaagaaactagagggaggtctaccctccaaagagataaatgatcctggtgaggtctgcaagtcttcagtaacatggactgagtgcatttgccaacctgcacagagctcagtagaagaaagcaaaaatcttactggcttgagaggtgaaaaggcaggatttgaggctaacaaaacaattggaaattcaaagagaattccagaaacaaagcaaccacaatgaagttgaatcacaaaatctgagtataaatgacccaaatacttggtcaacctctaaattacacagatgcaggacacacacccagaaacccctgctgaaaccacgtaagatactaagtggaaattctcaaacagaaaaaaaagaaaatgaaagtattctggaagaggtccacagaagttttgagatggcagaataaagaacaagcctgtgcctttgaatataaatctccagaatgtgctcaattctaaaatccaaaacacagattgaaataagattaaagaaaaaaatgaagagaacgtcatgaacccatcagagaatataaagcaggtgaccagatatgcaagtggatcctcgaaagaagacacaggatcagaaaaaaatatttgagaaaataatagttgaaagtttcccacatttggtgggggattttagctcatatctaaggtgttcaataaatgaaagcaaaataactgcagagaaaattacaaatagggtgaccaaggccttggcctggcctgaggcaacttagctagccccagggatgtgtgttctggtgtggagtaagatcagggagctaccagtgcaatctctgcctctacatcagcgggactgatcattttaattttgattgcagttactgctggtatctgaatcaaagtggtgatgcttcctgaggaaatttctgaagccattagtcaattagaacattataagccatgcctcccgtgaagaggagcatctaatcccagcatttgcaccagggaactctgcctgaagggattaccagtgacttgaaagtgtaaccaacaacaatcttgtcactattacgcaccagctaagcaatctgaacatacatgctgaagacatatttggtgagctatttaaggaggctaactacttccacattagatcaaattctcttagaatttgttgcctcatggtcaaagtcactcagctggattcagcagtggaagaggtgtcaccacaggacatcaacatggaaaaagccctcaaaagttccccagcccaagaccagcaagcagtttcaaagagcagcattcctactcctgctgctgacatttacaatcagagtgattgaccacctcccctgaatagcatgagagcacatggagatgataaagacaggctgaagttctatactgatccttcccatttatttgacctctggaaagaagaagtgctgcaggacacagaagacaaaaggaaagacagaaggcatcaaaagtagcaaaaccacatagatggcaccacttatgatgtgaaaaggattacaaaagccagaaacaagcaccatgagtggaatatgatggcatatgacaaagagcttagacccagcaacaggttgtctcagtgtgtaccatggagcatcctgcaagaacccctgtccctagacagcaggtcccatacaatggatgttagggatgctcttatcgagctactccaaaccattctcacgacccttcctaggcagctgaagatgcatgtccacatgggcccacaaaccaggcccctgagcttgagtgccacccctcctctgcctcagtgaggcacatggccctaaatggacctcaagagcggcccccacttccctgccttaccccagctgtgttccaatcctgtgcacttatggctccaggagactacaggatgctcccagcacaggtgcttgcatattacaacccgtcaagacctccttctcttcccccttttacagtacaaactgcctttgttgccttctgcaaatcctcctgccgcctccttcctcatatcagctggctccacacaccctcctcctcaccctccctccactggcccctgggtccagcattccctcctccagccacacactttccccaccaggcctcttggctcagttcttcttttttgtcttccccatgcattcgcccccacactgccaccagtagctgaggcaaagtgtcaagaacctgcacagccaccaaaaagtgatacgagaagtgatctccttgctgctactggaatggggatttgactggaaaaggtgcaggatctgtgtgagcaagaggccaagagtgagccccttgggaaggacaaggtcacaaccctgtcaatacattgcagtggagaagtacagggattctgagggagacttagagtttgacaaaaacaactggcccaaccaaggcagtgctggggctagctctatgcaacaggtgggtagctggtaccaggctgggtgacagccaggagaccatatgctaagaatgtacttaagatttcaagtggtctcagcactcaatggtattataatcccgcagctgagcaccttttgtattaatgatgtgatattgcttctgcacatccaaaaattctgggctttctcagtatttactgtgtaacatttaaatgccattaagcatagaatatcatgctgcacatgaagaaataggatgtaactattacatcgtcctgaaaagagcgtttttttttttcctgtaggccatgaaagtatttagtgtattttgggtttactcttattgatcattacaattctgcagatgtgctgtgtgaagctgcctggtgctaggcctttgaagattaataactgggtttcaggtggatatcttcagatgagtaattgatattgttttactgttttggggtgttttatgagtttgaaaagctgttggttctagagggaaaggtttaacaaataaggagaatccaatatttctgctttcattttcttggcgtggtatcctctgtgtgaatttgaatgctcagttgaatagttttattgccttagtacacacttgtcaaatgtaaagttggaaggacccttttcaggaagggtttcctgtgaacattcacttagtaaatgcttgtggatgattcttgtaagaggtctcagtgcttagaagtaatgaaaatgaagaacaagatcctttcaccttcaggggactttgggaccaatactcaaaatatttgccagcccttacagcttgggcaccagccagcaggagcagaggtgagtggtccaggagctgggcagctctgggcagagcagctgcatgtgggccctgggtgctaaagcaggcctgctcattgtgtgtcctggaagagctgtgttctaaaggaggccacaggagttcttagctccttagtgtgtgacagacagtgcttcagagggcactgtgctccacttggtcttaattttgaaagtttacattgaggtgaaagtagatattgacagctgttcactcttcatgtgctgtgttcagattacggatgaatccatgagcagggcctttgccttatcctagaaacacgtggttatcttccctcatgttgttgctggcacatcacatgttgataagtacctctcaagattaccttgatagacccgtagctgtagaagagcacttagttctcacctctgcccttccccttattcttgcaccgtggtcatgctagagacccatggggtttattttcctggctaaggcaaactgttgggaaatgcaagaatgagagcttctattgtgtaattctggatgaagaataactgcttgtttagaacaaataaatgtcccaggctttgattatagttatgacttcaaaaatatgctaagaccatttacttactgaggaaatcttacctacttatagtatttcaaattgattgcagtcattagtgactttcagttacgatgaagggaagaatcttgagctaaacgcagggttgtctgaggtgatgataggagtggaaacaggatggtaactaatggatttaggttaatgagttctgagaaaatcaaccagcccctgcaagtcacagctctgtgtctctaagtggacacctgtctgtcttgaggcagtcatcatataatacgcatcacatagattttagtcatatggattttgaaactctggaaaccatatgtctgcagctccctttcatgaccatgcttgtgctggtttatctgatctcctcagctgtaccagctgctgtggcatgagcttcttgagcttctctttttagagctgatcattatctggcggctgtcgctataattttcttgaatacaagtaaacattatttataaacttttaaatttctctgtttaatttttctctctcttttttttttaatgtagaggcctactttgctggcatctttgggtcctactcagtcataaaccaccctccccaccctagagtagccactcttgatttctaatacagatgagttctgccaggtctagttgaatggcattatacagtcaccatcatgaccctgggtctcatcatctcgctgtgtgtggtcgtagcctgttcactcccatagctgtccaatgttccactttataaataactcacatttcttgtcctttcactgttgatagacaattttgttgtatacagattttacaaatcgtgctgctatgaataatattttcatatagtttggcacacacatacatgcatttctattggtttagaactaagagtgctattgctggatcataggtgagtagatgattggcttagtctctcaaaaaccaagtttctagtagtaaacaaacagtagaggagggtgaatctcttacttagaaattattcagataataaatgaagaaggaatgagagactgagactataataactcttttgcaacccatcatgaattaacagatttagccattgaatagcaatggcaattaacaacacaataaaagaaaaaactaatgtgaggttcttcctcttgatggaagaacacaatacagcaccatcaaacaagtccaagtgaaaaagaaaccaagcctgaatagaagctgatctccatagccaattaccaatttagaggcaatacagataatagaaatacatattaaactatgccttggggagcaatcagcaaaatgcacactatgggaagctctaccacacaatataaatttcaaggaagaatctatagaataaagacaacaaaagcatatttctaaaggtaaaactaaatcataatattggatgataaaaataaaaataaaacaaagaagtgatgatcgtaaaagtcaggttgtgtttttatttgagggaagaaagagtttattgctgggctggagcaaatgatggggcttctggtttgccccacaaacatctatccctggtgattaatgggtgtttactttgtattaaaggacacaaattttagacatttttttcttttggagtcctatattttattttatgacacaaaggctaatgaaaaaataatttatagagtatggttactgttttgcacaaagcctcctccccatcttcttttctggacactgagcacccagaacaaccagcagccccaggacccccagcaggggtgacctcacttctaggtgtgcatgcagcttacatcaccagaggtaacatgcaggtctactccttgactcctggaagaacctgataggacacagctgaagggaagccttctccgccatctgtaggctcttggccatcagtgtagagggagcgggtcctcacttctccacaggcgtcttcatggccatagcctcctctctgcatggggagtaaggcctggcccttcgcctgaacacggtgacagggatctcgccaaaggtggagatcacaccattcctcctttagcaacctcacgcttcttttgctttacaggaaagttgactcaggctggtgtccagtaaagaaatcccaaggagaggccgggtgcggtggctcacgcctgtaatcccaggactttgggaggtcgaggcgggcggatcacgaggtcaggagattgagaccatcctggctaacacggtgaaaccccgtctctactaaaaaatacaaaacattagcagggcgtggtggcgggcacctgtagtcccagctactcggaaggctgagccagaatggcgtgaacccgggaggcggagcttgcagtgctcctgctcccaggaggcggatgcccaggatgaactttaaggtggagagaggaatgtcattgctcatcctccaggttccaagtaagaaccttcctgcccagcccatctcgatctccctgaatcctcaacgctaataaggactgtctccttctcacctccctagactgggctttttaacactggagagtggatgtgatttctagtttcagcatgtcctggtttattgtgctgccagtaaaataaaatcaaaatacacattgaataaataataaataacccatagtgagcaaaggcttacaatgtatttttttgagtcaagacctcactctggcctgggctggagcccagtggtgggatcgtagctcaatgcagccttgtatttatgagctcaagggatcttcccacctcagcctccacaatacctggaaatacaagcgcccaccaccaggcccagctgtttacttatttttatttttttagagacgagggtctcactatgttgtccaagctggtctctaattcctgatctcaagcaatcctcttgccttagcatcctgaattgctgggattacaggcatgagctgtcatgcccagcttgtaatgtactgacactatcaaggtttctttctccaagtgtggacacagcagcagacaccccttgtctcttgggtcaggacactgggtagagtggaagagcagggcaacaaagtcactgcagaaagcacccacgtggaagaggtccagcagggagagccacctgttccagggacaccatatttagggataactcctctttctgggcaggactgttctttgattacttttgtattcacaatagttctgaaatcgtaggatgatgagactcaagactggctagggttttattttggtttagtttttattttgttttgttttgtttttttcaaaaaaccatcatgagatttgttaatcttttgaatggtttttgtgtctcaatttccttcagaacttaactcttcttaacatttcacagtccactttcagttaaaaatgtactagtacccataaatagagaaatcttgcaaccatttatcctctataaaatgtccatctttatggtacagatattatatgtaatatatctatataggttataagtcccacaagaaaaaaacttcttcttatctttaaacagttctatgtatataaattagagtaaatgaggggaacaagatagccttttgcacttagctcagtatttaccatttttgatcatcttcatcttttctgataacctcatttcccacctggtatcatttgcctttagcctgaagaattttctttaccaattgttattgtgcagatctgctggtgacaaattctcttaattttcttttacctgaaacatctccttattttgcctgtccttgaaggacatctgtgctggatatagaattcttagttgtcctttttttcttccagcactttaaaggtgttattctactttcttttgtttctatggtttctaataagaagtcattggtcaggacaagaggaacagagaaaccaaaactgaggaaataacatggtagacctaaaacttatcaataatgaaaaaaaaacctatatgaatcaacactccaatgaagggcagagattgtgtgaattggttaaaaaaaaaaacaaaacacagaacccaaccatatgctctttccagatatgcagtttctttgtttcttttttttttaatttaacttttatttttagttaaggagtacatgtacaggtttgttatataggtaaacttgtgtcatgcggttttattgtacagattatttctgtacccattagtacccacccaagtattaagccttagtacccattagctatttttcctgatcctctccctcctcctctaccctcaagtggaccccagtgtgtgttgttcccttctgcgtgtccatgtgttctcatcatataactcctacttataagtgagaacatgtggtaattggttttctgttcctgcctcagtttgctaaggataatggcctccaactccatccatgttcctgcaaaggacatgatctcattcttttttatggctgcatggtattctgtggtgtatatgtgccacattttctttttcttttttttttaatggaaaataacttttattgagatcccaccagttgcaatatctgttcccagcattaagctccttcttcctttgcaattcggtctttcttgagtggtcccatgaatgctttcttctcctccgtggtctggaagcggccatggccaaacttggagggggtgtcaatgaacttaaggtcaatcttctccagagcccgctgctttgtctgcaccagcaaggacttgtggagggtgagcacccacttcttggttcccaccacacagcctttcagcatgacaaagtcattggtcacttcaccatagtggacgaagccacccaaagggttgatgctcttgtcagacaggtcatagtcagtggaggcattgttcttgatcagcttgccatccttgataaggtagccctagccaatcctatagatcttgttgatctcagtgcggtgatggtagcctttctgcccaccacgtaccacagagaagcccacacgagcaggatgccatgccccaatacaggccaccttgcacaggccttggtgggtcttgcggggcagcttcttggtgtgccaacgactggtgacccttttgtagcctttgcccttggtcaccctgatgacgtcgatcatctcatcctgcccaaacacttggttcacaggtacctggtgcttgagcctctcgccagcccagtccagcttctcagccacagtgcctccattcacctggatctccatcaggtgggccttcttctggcacagaggaagcaggtgcatctgggtgtgggcaatgacgcagatgacttggcagtacttcttcatgctgctgaagtccttctccagctgcttcttgccatcctcatcctgccatttcttgcagtacttggtaaaggccttcttcttagatttatgccagttcttatagaaacgcctcttgcattcatcactgatgtgctcagcgaagacagtcttgcaagtccggaagccttgaggggtttccacgcagcccacaatgcccactggtggcctctccacaatggtcacagcctccaccacctccttcttgttcaccttggatcctggcctgtcgacttcccgcacgatgtgggtcatgccagccttgtatcccaggaaggctgtgaggtggaccggcttggatgggtcatccttagggaagctcttcaccttcccacgatgcctgctgctgcgcttccaaggcaggaagccgagggacccatgtctgggagtggagaactttctgtgagacatcacgccatcaaatcctgccggtagagcatatttgccacattttctttatccagtctaccactgatggacatttaggttgattccatgtctttgctattgtgaatagtgctgcaatgaacatatgcatacgtgtaagacatgggctttctttttttaatttgctttgttttattttactttaggttccaggatacatgtgcagaacctgcaggtttgttacatacgtatagtctgccatggtggtttgctgcacctattgacccatcctctaagttcccgtcccttgccccccacactccccaaaaggccctgatatgtgttgttcccctccctgtgtccatgtgttgtcattgttcaactctcacttaggaatgagaacatgaggtgctttgttttctgttcccgtgttagtttgctgaggatgatgacttccagcttcatccatgtccctgcaaaggacatgaactcattcctttttctggctgtgtactattccatggtggtatgcaccacattttctttatccatctatcactgatggacatttgggttggttccaagtctttgctattgtaaatagtgctgcagtaaacatacgtgtgcatgtgtctttatagtagaatgatttatattcctttgggtagatacccaaagcgtcaaatagtatttctggttctcgatctctgaggaatggccacactgtcttccacaatggttgaactagtttacattcccaacaacagtgtaaaagtgttcctatttctccacagcctcaccagcatctattgtttcttgactttttaataattaccattctgactggcgggagatggtatctcattgtggctttgatttacatttctctaatgagcagtgatattgagcttttttaatttttctcattaagattttaatgtctcattttaaaaaattaaaggaagtttccatttattttttaagataaagatttagcgcacccaaatgcccccaaagccaacagaaaaatagctttgccctgtcatttccctaagaaagcactgcagttactcaaaatatgctcaaagaaaaaaatgcaatcctctgagttctaagtttcacgaaaggaccacatgttaaactatgtatatcgatttgatgtgtaagtatgcaataaatatgtacacatacattcctatctgcttcacatcattctagagtattcatagtatatgaagatgggatttagaaatgtgaaaaggctgtaacagtgaaaaggaaaaaaagggtacaatggtttttagaccagtagaataataatgcttagctagttaattattttaactttggagcagactaaaaaagttgttttgaaataaatggtacctgtaagtgatgcttactgcaatacttgttaagtgttatttgtcaccactgacaggaattatatagtgctagtaacacagctcccactgtcctagaattcctctaaatggccagctattgctaccattatcttgataagcatttcaagaattgtgttgctgagccttttaaaagataactagtttataacctatgtctccttatgtgcctacatcagttactcatggtcccaaagaagtataaaagaggatataagctgctgaagatttcacagaagaaacagagatatcagaaacttcatgatcatcaaacttaaaccaccgctgttttgctgcatttttacagtaggctgtgtaatggcctccatccagcccaccgcagtgatctgaaacagaaaacaaattatatttcttcaaattgttctttggaccaataacatactgtgacaaggcaagattttctaacaggaagtccacagatgtctgtaatttttgtttccacctgccattgtaggaaaaatgtttcagatgcactaaaagcacaggtggtaacttccagatttcttttttaagaatcccgtcgagctctgcaaagattgcagtaaaatctgttattatctatgagtttttcttctttagaaaataatctaaggcaatcctataatgtacatttacttgtggatgctatcagtagagacaaatacatgaaggcctcaagtgtcctagactttttgtgacgggtgaggcactgtactgtagatttgaattgaccctgaaaaagtgcaacaataatagactcatagagccgcttgtgtttctgccaggcatgttctgcagctttaaagtcattgagatgatcattattttcttctttatatgtcttccgattatcagttttatttagatcttcatggagaccatccattaggaacagaagaaattcttgtgaatcttgactgtatcctgcaaactggtaattgatcttcccaatggtgactttaaggtcttttggactgatatatctatactgtcctgtccacggggctttcatgattataccaaattcttctgccacttcacctttatgcccctaacaaatttgacttgttaatatcatcctgataacagtttcggttgaaataatcagccaaatgtggagcattacataggcactgcaatattgagttcatataacaagtatttcctaagttacgaagtccagtaagagctggtccagaacctccaaaaacaggattgagtttccaaatctgagaagcagaaagccttgagatctcagctttagggtagcatgttggcttgtcttcctgattaactgttggagttactgctggcttcctcttgaatagcctgggttatatctggggaggagtaagagcatttcagtttggaaggttccctatcctgcttagcaggaatctgtggcttgcctttatgagttggaggggtggaaggaggtgcagatgaaggagccatttccagtgggtacatatgaacagtgttagtggtgaatgataataatgaaacgttccagtgattggatcaagaaacttggcccagcctgaaggcagtcctggtaccatcctccccatttcttcacttcgtgctattatcgaaggttctcgttgagttttatttctttcagtatcatctgtatcctctctaaaagtttctgtccttagaattccagtttctggttgtcctttaatcttaaaaggcttgcctgaacctgaatcccctgtcgcagatgtatgggacacatctcttgttgactttttctgtacttctggggttgaacatcttctccccctgtcctctacagacttctcggcaccagaggtttcatgatcacttttactttgtctttttactcttgttatttctttgcccttctttgctgagatttctttatcctctttcttggcctgttcacgttctttcttctccatttcttcttttgctttttgttgcttctgtgtaatttcattttcttcagcttcttgtttctttttggctttttgctcgtgttcttccctcctcagtttctttctgtttcccttgctgtctttcctgaagttctttttcttgtttgtttttctccattagaagagcagtttctgcatgaatatgagccttttcttcatctgttaacatgagagttggagaggaaactactggcttggcggaacaatcaggaataactttttcattctgaggagactgttgctcatgatttgtactttcagatttgattatatgctcttcaggcaatttgactgccagttttttagtatgatcaatctgtggaacattctttatgctaggcactggctgaatgatgggtgaaacatcagatttagaagcagcaactgattcaactggaattgatatattcagtggtcctgtcctctcattttgatcattatcacttatttgatcacttatcaattctatgtcttcatccacttctataggtggaggtggcatctcggcagcaggtttagaaggaattgattcttccaatgagggataagtaaaatccaatgagatagacaactcttcattctggtgttgtgggggtggagtgaccttagcatttgttgtatactgggaatagcaaaggaaccagtttttatagcctccctctaaaaccaaaggcccaaggcccattgcacaggacagttttactttcccacttgaaaagtgcatctttcagatgccagagtgttgttccaatctgtaaatcttttgcagaactaaaccagtcaagaagtaccatatactccacattccccctcttcttccatgtatctatagaatcatctgggaggtgtgcttcaatccagctagcagtgactcctggactgatggcttttccaggaacactgagagaatgtaaaatacaggaatcctgataatcctgcattctttgagcatccattataatcaagctgatgtttttatccatcatcattgtgtatagttcctttgctgtgattgctcctttctctttggtctcacatttttcattcttttcaccattgctcttttgggttttgtctttggaatccaatacaatctccaaagaatctttagccaacgtgctgccatcctctcttcctgtttcctgccttttttgttgtagccactgtgcttccccctgcctgtccttttcctcaagttttttccagatttcaacttcttcatatcttagtttaaggctttcagagagtcgttcagtttctccagtggcttttttgatgtttgcaggtccaagtattgaatggaagtaatactgctgttgcttgaaatcagttcttttttggataggattataaacagtcatgtttcatatacatgtgtgagctttttttcatatgtttcttggccacgtaaatgtcttcctctgagaagtgtctgtttatatcctttgcccactttttgatggggatgggtttttttcttgtaaatttgtttaagttccttgtaaacaaacatgtgagctctcatcattcttgtttaaacacctaacagtcatcctaaccagtgcaacaaggaccaggcatggtggctcatgcctgtaatccctgcattttgggaggctgaggtgggaggatcacttgagatcaggagtttgagaccagcctgataagtgagacctcatctctaccaaataaaaataattttaaaagaaaatagatcaatggataagaaaggaagaaatgaaagtctttctttgtcaccagcttcattgtatatgtagaaaacactagggaattctgaaaaagtctctggaattaatcattgaatttgcaaaatagttcataaaatatatgtaataagtcgcttagatgaacatgaaaagatagcaaacaatactagtcatcaaagaagtgcaagttaaaaccacaatgagaaaccatcacacatcacctagagcaggtaaagttaaaaagacatatgataagtcttaacactggcaagaatatggaaaaaataggaatgtctgatattgctggtaggaatgcaaaaaatgtggcagccaatttgtaaagtggtatggcagtttcttatacagttacccatctattaccacatggcccagcaattccacaaatacgtatttatccaaaagaaataaaaatgtaagtccacacttgtaagcagttatttatagtggcttcattaataacaagccctaactggaggaatccacatgtctatcaactggagacacagaaaccaatgaataaactgggattccagcaatactcggcagctgctcagcaacaaaaatgaatgaatggcatcatctcaaacatcgttatgctaagggagagaccaaacaaaagactacataacatatgattgcatgtccatgaaattctagaaatgtcactattgcagtgacagaaagcacagcagtggttgaatgaagggaagggggtgagggtgggaggcaaggattaaatagaaaggggcataaagaaagtttttagggaaaagaaactgtcctctacagcgccacaactcaggagtgactgggagggggaagtaaggggagaagaaggtctgagggataaggggcagagagaagggctggggaagcaggaggagaggacgaggagcaggggaaaggactctaaagcagtggaggggcctagtaggaggatctttgcatttggtgtttctctactgggcagtgtggtagttacactataaataattaccaatatccaccaaaaagtgcagctaaaactggtgaatttttttacacgtaaacgccctgataagcaaaaaaaaaaaaaaaaaaaaaaaaaaaaaaaaaaaaaaagggggggggggaggaaagaaggcaaaaataaagacattgttagatcatcaaacccataaaattcatttcctaggggtcctgtactacatgtaattttaaaggacgttctacaggctgaagggaaatgatactagatggtgacccagatacacagaaaggaacaattaacaacagaaatgatgcacatacacatatcacatacacacccattttcttaatttcctgaagatacgtgactgcttgtctaaaacaaaaagtattacactgtattgttgagtttataacgtatattgatgtaacatatacaataataatagcataatgatagtttaaatgaaactacaccatttgaagtgtcctttattttgctggatgcagcttaatattacctgaatttcactgtgaaaagtcaaggaattgggtttcaattcttacaacaataaaaaattaatgtaaagaaatatagctaaaagccactaggagaattaaaaccataagctaaaaaatgtttacttgaaacataagaaagtagcaaaggaggaacagaaacaaaaagatatgagacaaattgaaaacgtatagcaaaatggtagaccaaaatccaaccattataagtgaagaaatgacacgacctgagtcacattagcaggactgctgagcactatggggagaacagacatgggcaggaagtgagggacagtgttagtgccacaattcaggagtgacagggtggcggggactaaaggggaaagagggtgtgagggatgagaggggcagacagaagggctggagaggcaggaggtgaggaaaaggagcagaggaaagaattctaaagcagtagaagagcctggcagggggttctttgcattcggtatttaatacattttgtgtgactgccttaaaactaatgggctccttatgatttttttttaaaaaggggttacaaaaatatcaagtgtccaaataaaatatgcacactgcttagatgtgcatagttccggaaaacgggcagtgctggagcgctggtgaagagcattgggactgcatggagccctcacaactttgaggtgatgactacaggctcccggttgcaatagacagtaacaaaccctgcttctttgtattcaggagatgttctggactcacacagggaaactctggctagagaatgaggataactttaaatgcaacaacccagagtcacagatccatagtctgcgaaagtaaaacaggagctttgagaatttaattgtaatgcagttttgacacaggtctttcacagattggaattctaatcattcagggattaccaatattgtgctacctactgtatcaataaacaaaaaggaaactggtctctatgagaatctctacctggtgctttcagacaaaacttcaccaggtttaaagagaaaactcctgactctacacgtccattcacagggcgagctcactgtctggcatcaagttccccatggtgagtttccctgtacaagagtccaaggggagaggtaagtgtcctttattttgctggatgtagtttaatattacctgaggtaaggtaaggcaaagagtgggaggcagggagtccagttcagggacggggattccaggagaagtgaaggggaaggggctgggcgcagcctgggggtctctccctggtttccacagacagatccttggccaggactcaggcacacagtgtgacaaagatgcttggtgtaggagaagaggaatcaggacgaagtcccaggtcccgggcggggctctcagggtctcaggctccaagggccgtgtctgcaatggggaggcgccgcgttgaggattctccactcccctgagtttcacttcttctctcccaacctgcgtcgggtccttcttcctgaatactcatgacgcgtccccaattcccactcccattgggtgtcggattctagagaagccaatcagcgtctccgcagtcccggttctgaagtccccagtcacccacccggactcagattctccccagacgccgagatgcgggtcatggcgccccgaaccctcatcctgctgctctcgggagccctggccctgaccgagacctgggccggtgagtgcggggttgggagggaatcggcctctgcggagaggagcgaggggcccgcccggcgagggcgcaggacccggggagcagcgcagggaggagggtcgggcgggtctcagcccctcctcgcccccaggctcccactccatgaggtatttctacaccgctgtgtcccggcccggccgcggggagccccacttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccagtgagtgaccccggcccggggcgcaggtcacgacccctcctcatcccccacggacggcccgggtcgccccaagtctcccggtctgagatccaccccgaggctgcggaacccgcccagaccctcgaccggagagagccccagtcacctttacccggtttcattttcagtttaggccaaaatccccgcgggttggtcggggcggggcggggctcgggggacggggctgaccgcgggggcggggccagggtctcacatcatccagaggatgtatggctgcgacgtggggcccgacgggcgcctcctccgcgggtatgaccagtccgcctacgacggcaaggattacatcgccctgaacgaggatctgcgctcctggaccgccgcggacacggcggctcagatcacccagcgcaagtgggaggcggcccgtgaggcggagcagctgagagcctacctggagggcctgtgcgtggagtggctccgcagatacctgaagaatgggaaggagacgctgcagcgcgcgggtaccaggggcagtggggagccttccccatctcctgtagatctcccgggatggcctcccacgaggaggggaggaaaatgggatcagcgctagaatatcgccctcccttgaatggagaatgggatgagttttcctgagtttcctctgagggccccctctgctctctaggacaattaagggatgaagtccttgaggaaatggaggggaagacagtccctagaatactgatcaggggtcccctttgaccactttgaccactgcagcagctgtggtcaggctgctgacctttctctcaggccttgttctctgcctcacgctcaatgtgtttgaaggtttgattccagcttttctgagtccttcggcctccactcaggtcaggaccagaagtcgctgttcctccctcagagactagaactttccaatgaataggagattatcccaggtgcctgtgtccaggctggcgtctgggttctgtgcccccttccccaccccaggtgtcctgtccgttctcaggatggtcacatgggcgctgttggagtgtcgcaagagagatacaaagtgtctgaattttctgactcttcccgtcagaacacccaaagacacacgtgacccaccatcccgtctctgaccatgaggccaccctgaggtgctgggccctgggcttctaccctgcggagatcacactgacctggcagtgggatggggaggaccaaactcaggacactgagcttgtggagaccaggccagcaggagatggaaccttccagaagtgggcagctgtggtggtgccttctggagaagagcagagatacacgtgccatgtgcagcacgaggggctgccggagcccctcaccctgagatggggtaaggagggggatgaggggtgatgtgtcttctcagggaaagcagaagtcctggagcccttcagccaggtcagggctgaggcttgggggtcagggcccctcaccttcccctcctttcccagagccgtcttcccagcccaccatccccatcgtgggcatcgttgctggcctggctgtcctggctgtcctagctgtcctaggagctgtggtggctgttgtgatgtgtaggaggaagagctcaggtagggaaggggtgaggagtggggtctgggttttcttgttccactgggagtttcaagccccaggtagaagtgtgccccacctcgttactggaagcaccatccacacatgggccatcccagcctgggaccctgtgtgccagcacttactctgttgtgaagcacatgacaatgaaggacagatgtatcaccttgatgattatggtgttggggtccttgattccagcattcatgagtcaggggaaggtccctgctaaggacagaccttaggagggcagttgctccagaacccacagctgctttccccgtgtttcctgatcctgccctgggtctgcagtcatagttctggaaacttctcttgggtccaagactaggaggttcccctaagatcgcatggccctgactcctccctgtcccctcacagggcattttcttcccacaggtggaaaaggagggagctgctctcaggctgcgtgtaagtgatggcggtgggcgtgtggaggagctcacccaccccataattcctcttgtcccacatctcctgcgggctctgaccaggtctttttttttgttctaccccagccagcaacagtgcccagggctctgatgagtctctcatcgcttgtaaaggtgagattctggggagctgaagtggtcgggggtggggcagagggaaaaggcctaggtaatggggatcctttgattgggacgtttcgaatgtgtggtgagctgttcagagtgtgatcacttaccatgactgacctgaatttgttcatgactattgtgttctgtagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggacagacagcccacccccgtgtccaccgtgacccctgtccccacactgacctgtgttccctccccgatcatctttcctgttccagagaagtgggctggatgtctccatctctgtctcaacttcatggtgcgctgagctgcaacttcttacttccctaatgaagttaggaacctgaatataaatttgttttctcaaatatttgctatgaagggttgatggattaattaaataagtcaattcctggaagttgagagagcaaataaagacctgagaactttccagaatccgcatgttctctgtgctgagtctgttgcaggtgggggtggggaaggctgtgaggagccgagtgtggacgggggcctgtgcctagttgctgttcagttcttcatgggctttatgtagtcagtccttagctgggtcaccttcactgctccattgtccttgtcccttcagtggaaacttgtccagcaggagctgtgaccacagaggctcacacatcgcccagggcagcccctgcacacgggagtccctgtgctttctgagacaaattttcagacccattcagctcctgccctccttatagggctcctcttctgctttggtctcctgccctctctcccttccctgattccagtgatcttcgtgctgactccaatcccaactcatgaatctaaagcagagcctaatttagatttgtatttgtttgtaaaattgggtccatagtctagaattgttccttcctgaagagagaaacctgatcgtgtgctgcagcgtgcagggcggttggtgtgggaggagggataggggagggaggacacacaagcagccctgctgagaaaagtacaggcggcctcggtgtcagtgtgaggggaccttgtgctgcagctgccacaaaacagcacttggcctgaggctatgttaataaagatactggctttagaataggaggtgctctacactgatcattcaactgacctttgttgtcagccagacacaggacagaaaagttctgcatctggggaacaccattgaagtaaaatcagaaaaatatctgagcatatgctccagtggtaagaggcagacgatacatacactataaccacagtaagaaaagaaagtgatggaaggtggtaagtgccatgaggcaggtgatccgggtatgggcagtggggacagggaaggtggctgttggacaggagttgtcaatgtgtgccttgttgcaaagatgacctttgaggaaagatttgagggacatgaggatgtctggggaagttctttctaggcaaggaaacacaacttcgtatagaaaggtatatacgaagttgt

SEQ ID NO: 2 - DNA, 6075 bp, synthetic construct
  source 1..6075: mol_type = other DNA
gaagttcctatactctagaaagaataggaacttcgtcgacccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcttagtccccatcctcgagcagcctcaccatggccccagtctcagagtccaggtttctgatgtagttcatcacaatgttcacattggtccagccaccagcctgcatgatttcagggatggacacaccagccctggccatgtccctggcagcacccactctggcagagtggccagaccaggccaggtatctctgcccagagtcatccttggcaccatagatcaggcggtgggtggcctcaaagatcccttccagggcccgggtggacagttgggaggtggcagaaggggcagccacaccattctttctgacccggcagaacaggtagttgttggggtcatcagccacaccagacacagagatccatctctccaccagcttggtaacccccagggacagggccttctccacaccagctgtggacaccagggtcttggtcctgccaatgtggatcagcattctcccaccatcggtgcgggagatgtccttcactctgattctggcaatttcggcaatgcgcagcagggtgttgtaggcaatgcccaggaaggccaggttcctgatgtcctggcatctgtcagagttctccatcagggatctgacttggtcaaagtcagtgcgttcaaaggccagggcctgcttggctctctccccagcatccacattctcctttctgattctcctcatcaccagggacacagcattggagtcagaagggcgaggcaggccagatctcctgtgcagcatgttgagctggcccaggtgctgttggatggtcttcacagccaggcctctggcttgcaggtacaggaggtagtccctcacatcctcaggttcagcagggaaccatttcctgttgttcagcttgcaccaggcagcccaggatctgcacacagacaggagcatcttccaggtgtgttcagagaaggcctgcctgtccctgaacatgtccatcaggttcttcctgacttcatcagaggtggcatccacagggagggcaggcaggttttggtgcacagtcagcaggttggagactttcctcttcttcttgggcaccatggtggcgaattctttacgagggtaggaagtggtacggaaagttggtataagacaaaagtgttgtggaattgaagtttactcaaaaaatcagcactcttttataggcgccctggtttacataagcaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgaggggatcctgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccggagacgctgcagacccgcgacccggagcagctcggaggcggtgaagtcggtggctttccttctctctagctctcgctcgctggtggtgcttcagatgccacacgcgtcccgggggcccggttctccgctcccctcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcatgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgggatctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggctcaggcgccaccaactttagcctgctgaagcaggcaggcgacgtcgaggagaaccccggccctataacttcgtatagcatacattatacgaagttatccaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaatggcagtggagagggcagaggaagtctgctaacatgcggtgacgtcgaggagaatcctggcccagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgttgaccgcaagttggacatcacctcccacaacgaggactacaccgtggtggaacagtacgaacgctccgagggccgccactccaccggcggcatggacgagctgtacaagtgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatgcacaacttcgtatagaaaggtatatacgaagttgt

SEQ ID NO: 3 - DNA, 95 bp, synthetic construct
  source 1..95: mol_type = other DNA
  modified_base 1^2: mod_base = OTHER; note = phosphorothioate internucleotide linkage
  modified_base 2^3: mod_base = OTHER; note = phosphorothioate internucleotide linkage
  modified_base 93^94: mod_base = OTHER; note = phosphorothioate internucleotide linkage
  modified_base 94^95: mod_base = OTHER; note = phosphorothioate internucleotide linkage
tataattttgcttttaacatatattcaaatcctttgatactgttcctttcatatgggctttagaatagggaggtgctctacagtgatcattcatt

SEQ ID NO: 4 - DNA, 100 bp, synthetic construct
  source 1..100: mol_type = other DNA
  modified_base 1^2: mod_base = OTHER; note = phosphorothioate internucleotide linkage
  modified_base 2^3: mod_base = OTHER; note = phosphorothioate internucleotide linkage
  modified_base 98^99: mod_base = OTHER; note = phosphorothioate internucleotide linkage
  modified_base 99^100: mod_base = OTHER; note = phosphorothioate internucleotide linkage
gatgcacatacacatgtcacattcacactcattttcttaatttcctgaagggcaaggaaactccagtccaaatgcacaatggcaggaaggtgtctgtgtt

SEQ ID NO: 5 - DNA, 10846 bp, synthetic construct
  source 1..10846: mol_type = other DNA
ttattatcatgacattaacctataaaaataggcgtatcacgaggccctttgtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtggtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgcgagaccttcctggtctcagaagttcctatactctagaaagaataggaacttcgtcgacccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcttagtccccatcctcgagcagcctcaccatggccccagtctcagagtccaggtttctgatgtagttcatcacaatgttcacattggtccagccaccagcctgcatgatttcagggatggacacaccagccctggccatgtccctggcagcacccactctggcagagtggccagaccaggccaggtatctctgcccagagtcatccttggcaccatagatcaggcggtgggtggcctcaaagatcccttccagggcccgggtggacagttgggaggtggcagaaggggcagccacaccattctttctgacccggcagaacaggtagttgttggggtcatcagccacaccagacacagagatccatctctccaccagcttggtaacccccagggacagggccttctccacaccagctgtggacaccagggtcttggtcctgccaatgtggatcagcattctcccaccatcggtgcgggagatgtccttcactctgattctggcaatttcggcaatgcgcagcagggtgttgtaggcaatgcccaggaaggccaggttcctgatgtcctggcatctgtcagagttctccatcagggatctgacttggtcaaagtcagtgcgttcaaaggccagggcctgcttggctctctccccagcatccacattctcctttctgattctcctcatcaccagggacacagcattggagtcagaagggcgaggcaggccagatctcctgtgcagcatgttgagctggcccaggtgctgttggatggtcttcacagccaggcctctggcttgcaggtacaggaggtagtccctcacatcctcaggttcagcagggaaccatttcctgttgttcagcttgcaccaggcagcccaggatctgcacacagacaggagcatcttccaggtgtgttcagagaaggcctgcctgtccctgaacatgtccatcaggttcttcctgacttcatcagaggtggcatccacagggagggcaggcaggttttggtgcacagtcagcaggttggagactttcctcttcttcttgggcaccatggtggcgaattctttacgagggtaggaagtggtacggaaagttggtataagacaaaagtgttgtggaattgaagtttactcaaaaaatcagcactcttttataggcgccctggtttacataagcaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgaggggatcctgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccggagacgctgcagacccgcgacccggagcagctcggaggcggtgaagtcggtggctttccttctctctagctctcgctcgctggtggtgcttcagatgccacacgcgtcccgggggcccggttctccgctcccctcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcatgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgggatctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggctcaggcgccaccaactttagcctgctgaagcaggcaggcgacgtcgaggagaaccccggccctataacttcgtatagcatacattatacgaagttatccaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaatggcagtggagagggcagaggaagtctgctaacatgcggtgacgtcgaggagaatcctggcccagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgttgaccgcaagttggacatcacctcccacaacgaggactacaccgtggtggaacagtacgaacgctccgagggccgccactccaccggcggcatggacgagctgtacaagtgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatgcacaacttcgtatagaaaggtatatacgaagttgtagagaccaacctggtctcagctagctccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaacca

SEQ ID NO: 6 - DNA, 10846 bp, synthetic construct
  source 1..10846: mol_type = other DNA
ttattatcatgacattaacctataaaaataggcgtatcacgaggccctttgtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtggtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgcgagaccttccgggtctcagaagttcctatactctagaaagaataggaacttcgtcgacccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcttagtccccatcctcgagcagcctcaccatggccccagtctcagagtccaggtttctgatgtagttcatcacaatgttcacattggtccagccaccagcctgcatgatttcagggatggacacaccagccctggccatgtccctggcagcacccactctggcagagtggccagaccaggccaggtatctctgcccagagtcatccttggcaccatagatcaggcggtgggtggcctcaaagatcccttccagggcccgggtggacagttgggaggtggcagaaggggcagccacaccattctttctgacccggcagaacaggtagttgttggggtcatcagccacaccagacacagagatccatctctccaccagcttggtaacccccagggacagggccttctccacaccagctgtggacaccagggtcttggtcctgccaatgtggatcagcattctcccaccatcggtgcgggagatgtccttcactctgattctggcaatttcggcaatgcgcagcagggtgttgtaggcaatgcccaggaaggccaggttcctgatgtcctggcatctgtcagagttctccatcagggatctgacttggtcaaagtcagtgcgttcaaaggccagggcctgcttggctctctccccagcatccacattctcctttctgattctcctcatcaccagggacacagcattggagtcagaagggcgaggcaggccagatctcctgtgcagcatgttgagctggcccaggtgctgttggatggtcttcacagccaggcctctggcttgcaggtacaggaggtagtccctcacatcctcaggttcagcagggaaccatttcctgttgttcagcttgcaccaggcagcccaggatctgcacacagacaggagcatcttccaggtgtgttcagagaaggcctgcctgtccctgaacatgtccatcaggttcttcctgacttcatcagaggtggcatccacagggagggcaggcaggttttggtgcacagtcagcaggttggagactttcctcttcttcttgggcaccatggtggcgaattctttacgagggtaggaagtggtacggaaagttggtataagacaaaagtgttgtggaattgaagtttactcaaaaaatcagcactcttttataggcgccctggtttacataagcaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgaggggatcctgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccggagacgctgcagacccgcgacccggagcagctcggaggcggtgaagtcggtggctttccttctctctagctctcgctcgctggtggtgcttcagatgccacacgcgtcccgggggcccggttctccgctcccctcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcatgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgggatctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggctcaggcgccaccaactttagcctgctgaagcaggcaggcgacgtcgaggagaaccccggccctataacttcgtatagcatacattatacgaagttatccaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaatggcagtggagagggcagaggaagtctgctaacatgcggtgacgtcgaggagaatcctggcccagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgttgaccgcaagttggacatcacctcccacaacgaggactacaccgtggtggaacagtacgaacgctccgagggccgccactccaccggcggcatggacgagctgtacaagtgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatgcacaacttcgtatagaaaggtatatacgaagttgtagagaccaaccgggtctcagctagctccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaacca

SEQ ID NO: 7 - DNA, 8463 bp, synthetic construct
  source 1..8463: mol_type = other DNA
ttattatcatgacattaacctataaaaataggcgtatcacgaggccctttgtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtggtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgcgagaccttcctggtctcagaagttcctatactctagaaagaataggaacttcgtcgacccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccggagacgctgcagacccgcgacccggagcagctcggaggcggtgaagtcggtggctttccttctctctagctctcgctcgctggtggtgcttcagatgccacacgcgtcccgggggcccggttctccgctcccctcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcatgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgataacttcgtatagcatacattatacgaagttatccaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaatggcagtggagagggcagaggaagtctgctaacatgcggtgacgtcgaggagaatcctggcccagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgttgaccgcaagttggacatcacctcccacaacgaggactacaccgtggtggaacagtacgaacgctccgagggccgccactccaccggcggcatggacgagctgtacaagtgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatgcacaacttcgtatagaaaggtatatacgaagttgtagagaccaacctggtctcagctagctccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaacca

SEQ ID NO: 8 - DNA, 38498 bp, synthetic construct
  source 1..38498: mol_type = other DNA
gggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtcctagggcggccgcataacttcgtatagcatacattatacgaagttatccggaaccgagtacaagcccacggtgcgcctcgccacccgcgacgacgtccccagggccgtccgcaccctcgccgccgcgttcgccgactaccccgccacgcgccacaccgtcgatccggaccgccacatcgagcgcgtgaccgagctgcaagaactcttcctcacgcgcgtcgggctcgacatcggcaaggtgtgggtcgcggacgacggagccgctgtggcggtctggaccacgccggagagcgtcgaagcgggggcggtgttcgccgagatcggcccgcgcatggccgagttgagcggttcccggctggccgcgcagcaacagatggagggcctcctggctccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccgcatgccccacgctactgcgggtttatatagacggtccccacgggatggggaaaaccaccaccacgcaactgctggtggccctgggttcgcgcgacgatatcgtctatgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggtatgccttatgccgtgaccgacgccgttctggctcctcatatcgggggggaggctggtagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggccctgccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgcgagatgggggaggctaacgagctcggcagtggagagggcagaggaagtctgctaacatgcggtgacgtcgaggagaatcctggcccaagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgacagaaaccaagacaagcgtgtccaggcacatctactttttcagcgccagaggcaggatcgaccccctggtgtacctggacgagtttctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggcagcatcagataccccgcctggaacggcatcatcagccaggaggtgctggactacctgagcagctacatcaacaggcggatcaccggttgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctggctccggcgagggcaggggaagtcttctaacatgcggggacgtggaggaaaatcccggcccagtgagcaagggtgaggaggataacatggcctctctcccagcgacacatgagttacacatctttggctccatcaacggtgtggactttgacatggtgggtcagggcaccggcaatccaaatgatggttatgaggagttaaacctgaagtccaccaagggcgacctccagttctccccctggattctggtccctcatatcgggtatggcttccatcagtacctgccctaccctgacgggatgtcgcctttccaggccgccatggtagatggctctggataccaagtccatcgcacaatgcagtttgaagatggtgcctcccttactgtgaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggaactcaagcactccaagaccgagctgaacttcaaggagtggcaaaaggcgtttaccgatgtgatgggcatggacgagctgtacaagtgaattcgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggctcgagggaagttcctatactctagaaagaataggaacttcaaaggaagtgcttcagggtgaaggaaaatgatactagatggtgacccagatatacagaaaggaacaattaacaacagaaatgatgcacatacacatgtcacattcacactcattttcttaatttcctgaagacatatggctacttgtttaaaacaaaaagtattacactgtatcattgagcttataatataggttgatgtaatatatacaacaataatagcacaatggtaggttacaagaaactacactgttagaagtgtcctttattttctggatgcagcttaatataacctgaacttcactgtgaaaagtcaaggaatcgggtttccattctttgctattgtgtaaagaaatatagccaaaagccactaggagaattaaaaccataaactaaaaaatgtttatttgacacataagaaagtagtaaaggaggaacagaaacaaaaagatatgagacaaattgaaaacgtatagcaaaattgtagaccaaaacccaaccattgcaagtgaagaaatgacatgacctgagtcacattagcaggactgctgagcactgtggggagaacagatatgggcaggaggtgagggacagtgttagtgccacaattcaggagggaaagggtggcagggactaacgggggtggtgtgaaggatgagaagggcagacagaagggctggagaggcaggaggagaggaaaaggagcaggagaaagaattctaaagcagtggaagagcctggcagagggttctttgcattcggtatttaatacattttgttggacttcctaaaaactaattggctccttatgattaaaaaaaaaagagttacaaaaataccaagtgttcagataaaatatgcacactgcttggatgtgcagagttcaggaaaacaggcagtgcttgagcgctggtgaagagcattgggactgcatggagccctcgcaactttgaggtgatgactacaggttcccggttgcaatagacagtaacaaaccctgcttctttatattcaggagatgttctggactcacacagggaaactcagggtggggaatgaagataattttaaatgcaacaacccagagtcacagatccatagtctgggaaagtaaaacttaggagctttgagagtttaattgtaatgctgttttgacacaggtcttttgcagattggaattctaatcattcagggattaccaatattgtgctacctactgtattaataaacaaaaaggaaactggtctctatgagaatctctgtgtggtggcttcagacaaaacttcgccaggtttagagagaaaacccctgtctctacacctccattcccagggcgagctcactctctggcatcaagttctccgtgatcagtttccctacacaagantccaagaggagaggtaaggagtgagaggcagggagtccagttcagggacagggattccaggaggagaagtgaaggggaagcgggtgggcgcagcctgggggtctctccctggtttccacagacagatccttgtgcaggactcaggcagacagtgtgacaaagaggcttggtgtaggagaagagggatcaggacgaagtcccatgtcccggacggggctctcagggtctcaggctccgagggccgcgtctgcattggggaggcgcagcgttggggattccccactcccacgagtttcacttcttctcccaacctatgtcgggtccttcttccaggatactcgtgacgcgtccccatttcccactcccattgggtgtcgggtgtctagagaagccaatcagtgtcgccggggtcccagttctaaagtccccacgcacccacccggactcagagtctcctcagacaccgagatgcgggtcacggcaccccgaaccctcctcctgctgctctggggggccctggccctgaccgagacctgggcgtgagtggcttaatggaaaggtggtttgggcctctgtggggaggagcgaggggaccgcaggcgggggcgcaggacccggggagccgcgccgggaggagggtctggcgggtctcagcccctcctcgcccccaggctcccactccatgaggtatttctacaccgccatgtcccggcccggccgcggggagccccgcttcatcgcagtgggctacgtggacgacacgcagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccggtgagtgaccccggcccggggcgcaggtcacgactccccatcccccacgtacggcccgggtcgccccgagtctccgggtccgagatccgcctccctgaggccacgggacccgcccagaccctcgaccggcgagagccccaggcgcgtttacccggtttcattttcagttgaggccaaaatccccgcgggttggtcggggcggggcggggctcgggggacggggctgaccgcggggccggggccagggtctcacacttggcagacgatgtatggctgcgacctggggccggacgggcgcctcctccgcgggcataaccagttagcctacgacggcaaggattacatcgccctgaacgaggacctgagctcctggaccgcggcggacaccgcggctcagatcacccagcgcaagtgggaggcggcccgtgaggcggagcagctgagagcctacctggagggcacgtgcgtggagtggctccgcagatacctggagaacgggaaggagacgctgcagcgcgcgggtaccaggggcagtggggagccttccccatctcctataggtcgccggggatggcctcccacgagaagaggaggaaaatgggatcagcgctagaatgtcgccctcccttgaatggagaatggcatgagttttcctgagtttcctctgagggccccctcttctctctaggacaattaagggatgacgtctctgaggaaatggaggggaagacagtccctagaatactgatcaggggtcctctttgacccctgcagcagccttgggaactgctacaaaaggccagagcttgttctctgcctcacactcagtgtgtttggggctctgattccagcacttctgagtcactttacctccactcagatcaggagcagaagtccctgttccccgctcagagactcgaactttccaatgaataggagattatcccaggtgcctgcgtccaggctggtgtctgggttctgtgccccttccccaccccaggtgtcctgtccattctcaggctggtcacatgggtggtcctagggtgtcctatgagagatgcaaagcgcctgaattttctgactcttcccatcagaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatggggtaaggagggggatgaggggtcatatctcttctcagggaaagcaggagcccttctggagcccttcagcagggtcagggcccctcgtcttcccctcctttcccagagccatcttcccagtccaccatccccatcgtgggcattgttgctggcctggctgtcctagcagttgtggtcatcggagctgtggtcgctactgtgatgtgtaggaggaagagctcaggtagggaaggggtgaggggtggggtctgggttttcttgtcccactgggggtttcaagccccaggtagaagtgttccctgcctcattactgggaagcagcatccacacaggggctaatgcagcctgggaccctgtgtgccagcacttactctcttgtgcagcacatgtgacaatgaaggacggatgtatcaccttgatggttgtggtgttggggtcctgatttcagcattcatgagtcaggggaaggtccctgctaaggacagaccttaggagggcagttggtccaggacccacacttgctttcctcgtgtttcctgatcctgccttgggtctgtagtcatacttctggaaattccttttgggtccaagacgaggaggttcctctaagatctcatggccctgcttcctcccagtcccctcacaggacattttcttcccacaggtggaaaaggagggagctactctcaggctgcgtgtaagtggtgggggtgggagtgtggaggagctcacccaccccataattcctcctgtcccacgtctcctgcgggctctgaccaggtcctgtttttgttctactccagccagcgacagtgcccagggctctgatgtgtctctcacagcttgaaaaggtgagattcttggggtctagagtgggcgggggggtggcggggggtggggagggggcagaggggaaaggcctgggtaatggagattctttgattgggatgtttcgcgtgtgtggtgggctgtttagagtgtcatcacttaccatgactaaccagaatttgttcatgactgttgttttctgtagcctgagacagctgtcttgtgagggactgagatgcaggatttcttcactcctcccctttgtgacttcaagagcctctggcatctctttctgcaaaggcacctgaatgtgtctgcgtccctgttagcataatgtgaggaggtggagagacagcccacccccgtgtccactgtgacccctgttcccatgcttgcttctagagctccttttctcacacatcgcccagggtggcccctgcacacgggggtctctgtgcattctgagacaaattttcagagccattcacctcctgccctgcttctagagctccttttctgctctgctctcctgccctctctccctgccctggttctagtgatcttggtgctgaatccaatcccaactcctgaatctgtaaagcagagtctaatttagacttacatttgtctgtgaaattggacccatcatcaaggactgttctttcctgaagggagaacctgattgtgtgctgcagtgtgctggggcagggggtgcggggagggggttgctgtagaaagagggatggggagggaaaggaagtgcttcagggtggcctgaggctatgttaataaagatactgccttcaaaatagggaggtgctctacagtgatcattcattcaactgacctttgtcattggccagacataggacagaatggttctgcatctggggaacaccactgaagtaaaatcagaaaaatctctggccttttgtagcaggcgcgccgaagcttgctcctcaattccacatgcttatcatagatatcaagcagtgcccttgctgtctgcccatcttttctgaccctgggacaccaccctttatttactttccccacaactccttgaaggtcaagcccccttggctgatactctgggttatcatagtaaatgtgccctctatcttttgcaggtcactggaaaggagggttcctaaagcattcactcctgaccctgagggaggtgggtgcactcactccaggactcaggttcaccacagataagcaaaaaagtcctcacattcaaaattgtcctgggccacatgcagcccacgagccgcgggttggacaagcctgcatctagaacgtatgcaccataaccccagacatttttctgtttttactttaaggatgctttctaaatgcaaaagcagtcatatccctagcagacaacaaatgtcagttgaatgaatgatcactgtagagcacctccctattctaaagcccatatctttattaatgtagccacagggcaaatgctgttttgtggcagctaaagaacataagttcacgtggacatccatgccgccagtgcttttctcaccagggctgcttgtgtgtcctccctcctcccacaccaaccctcctgcacacttcagcacaaaactatatatttctcttcaggaacaataaccctagccttatgggtacaattttccaaccacatatgaatctaaattagactctgctttataaatctatgagtttggattggagccagcactaggattactacaactcagagcaggaagaagagtaggagggcagaagaggagttccaacagaaagttaaccatgatgagaaactatgggacccctcctctctgcaaatgtcacaatctggttcctttaaaaagattggagaaaaatcgaaaataccattaaggaaaaagccctgggaggaaggcaagacctggacaggtctgaaaatttgtctcttgataccacaaggagatttgtatgcagggaccaccctaggtgacatccaactccctgtgatcacaggtcttggtgggacaaggttctactgaagggccaaggacaatggagcagcaaagatgacccaggtgagctgtgaccacataaagcccatggtggccagaacagaaactgggcacagccccatctactctcctcccctgcaacaaatcagcacaagaaacacgtggactctggaaggttctcatgtcttccatttattttgtctctcaaattttaggaatcttctcctttaattaactcatcaacctctcatggcaagaatttgagaaagtaaatttatactcaggttctaattttaatagggaaggaagaagttacagctcagtgcaccatgaagttgagacagagatggagacacctcagccccacctctctggaacaggaaagatgattggggagggagcacaggtcagcgtgggaagagggtcatggtggacatgggggtggggtggtctccccacctcctcacattatgcctacacgaacacagacacatgcaggtgcctttgcagaaacaaagtcagggttcttcaagtcacaaagggaagggcaggaacaactcttgcctctcagtcccacacaaggcagctgtctcacactatagaaaaaaatattcatgaacaaattcatatccatcacagtgaggggtcacaccttaaacagcccatcgcatgctcaatacatccaatgcaaagaaaccccatagcacagctgtgtccactgttccgcccaacacccaacacacattaggtcctccaagctctcacctttacaagctgtgagagacacatcagagccctgggcactgtcactgcctggggtagaacaaaaacagaacctggtcagatcccacagaagatgtggctagaggaggaattgtggggtgggtgagctcccccatgggctcccaaacacaatatcccaaggacctcaggcatcagcctccttcatacttacttgcagcctgagtgtaactccctccttttctatctgtgagaagaaaatgtcctgtgagataccagaaaggagccagggccttaaggtcctagaggaacctcctagtcttggaccccagagaagtttccagaaatgtgtgactgcagacccagggcgggatcaggaaacatgaagaaagcaggtgtgggtcctggaccaatagccctcctgaggtctgtcctcagggaccttcccctgtgacttgtgactgctgggatcaggtcccatcaccgccgtaatcaaggtgataaatctgtccttcattttaacaggtgctttacaaaagagtaagtgctggcacacagggcccaggctgggtaggcccatgattgtggatggtgcttcccagtaatgagacagggcacatttctagctggggcttggaaccctcagtgagacaagaaatctcagaccccacccttcaccccttctccacctgagctcttcctcctccacatcacggcagcgaccacagctccagtgatcacagctccaaggagaaccaggccagcaatgatgcccacgatggggatggtgggctgggaagacagctctgggaaaagaggggaaggtgaggggccctgaccctgctaaaggtctccagagaggctcctgctttccctaagagacatgacacccccatctccctccttaccccatctcagggtgaggggcttgggcagaccctcatgctgcacatggcaggtgtatctctgctcctctccagaaggcaccaccacagccgcccacttctggaaggttccatcccctgcaggcctggtctccacgagctccgtgtcctgggtctggtcctccccatcccgctgccaggtcagtgtgatctccgcagggtagaagcccagggcccagcacctcagggtggcctcatggtcagagatggggtggtgggtcatatgtgtcttgggggggtctgacgggaagagtcagaaaattcaggcattttgcatctgtcatgggacactccaccagcacgcatgtggccatcttgagaatggacaggacacccgggatggggaagagagcacagaacccagacaccagcctggacacaggcacctgggataatcttctattccctgagaagggaacagcgacttctggtcctgacctgagtggaggctgagggactcagaagtgctggactcagacccccacacacattgagtgtgaagcagagaacaaggcctgagaggaaaagtcacgggcccaaggctgctgccggtgtcaaagggaaccactcatcagtattcgagggatcgtcttcccgtcactccttcagagattttatcccttaattgtgtcagagagcagggcggaacctcagagtcactctctggtacaggatctggaacccaggaggattcctctccctcaggaccagagggagggtgatattctagtgttggtcccaattgtctcccctccttgtgggaggccagcccgggagatctacaggcgatcaggaaggcgccccgtggcccctggtacccgtgcgctgcagcgtctccttcccgttctccaggtatctgcggagccactccacgcacgtgccctccaggtaggctctcaactgctccgcccaacgggccgcctcccacttgcgctgggtgatctgagccgccatgtccgccgcggtccaagagcgcaggtcctcgttcagggcgatgtaatccttgccgtcgtaggcgtgctgttcatacccgcggaggaagcgcccgtccgaccccacgtcgcagccatacattatctggatggtgtgagaacctggccccgaccccgcggtcagcccagtcccccgagccccgcccagccccgaccaacccgggggaatttttggcctaaactgaaaatgaaaccgggtaaaggcgcctgggcctctcccgggtcaagggtctcggggtcccgcggcttcggggtggatctcggacccggagactgtgggcgacctggcccgtccgtgggggatgaggggtcgtgacctgcgcccccggccggggtcactcaccggcctcgctctggttgtagtagccgcgcagggtccccaggtccactcggtcagtctgtgactgggccttcacattccgtgtctcctggtcccaatactcaggcctctcctgctctatccacggcgcccgcggctccatcctctggctcgcggcgtcgctgtcgaaccgcacgaactgcgtgtcgtccacgtagcccactgcgatgaagcggggctctccactgccgggccgggacacggatgtggagaaatacctcatggagtgggagcctgggggcgagcagtggctgagacctgaccgaccctcctcccggcgcggctcccccggtcctgcgcccccgccaggagggccccttgcttctccccgcagaggcggtttccctcccgaccccgcactcacccgcccaggtctgggtcagggccagggcccccgagagtagcaggaggagggttcggggcgccatgacggccatcctcggcgtctggggagaatctgagtcccggtgggtgcgtgcgggctttagaacagcgaccgcgacgacactgattggcttctctggaaacccgacacccaatgggagtgagaactgggtccgcgtcgtgagtatccaggatgaaggaccctacgtaggttgggagagggagaaaagaaactgcggagttggggaatccccaaggctgtgactccccaatccatacaccgccttcggggcctgagaccctgagagccacgcctggggccctgggacttcgccctgacccctctgctcctgtgcgaagcgctctgtctcaatgtctccctgagtcttggcccaagagctgtctgagaaacaagggagaaaccctcggttcacttttcatcccattatctctgtccctgaactggactccctgactcccactccttacctgtccacctggactcttctagaagaaaaatcaccccatggagtttggtgccagagagtaagcttgctctgggaatggaggtgtagatttttgttgttgttgtttttgtttttaatccggaaaagttgtgcctgagtgcatgagacagaatagagaccaatttgctctttgtttattaactacagtgggtagcagaatcttggtaacccctgaatgatcaggaatctaataggtaaaaaatgtgactttggccccttaatgtataaatgtgtccaaaagcattacaacaggactcacaaagctactaagttgcactttcccagacaatgtatctgtgactcctgcttgtcttactttaaatttgccatcattccttagctctgagtttctgtgtgagtccacgacatctcctcaatacaaagtagccactgtgttcctatatgttgcaaccagaagccattacaggctttattcacctcaaagttgcaactgttcaaggcagtcacaatgcccctcactagtgctcatgcactgcctgtttttaggaagtatccacttctaagtgttgtgtatattttataggaacacttagtaatttttaaacctgattaacataaaaaatagtttttaggcagacccacatgaggtattaaaagacaactgccaaggacaccctgctaggctctgtagattgatgtattaaaatccataaaacaatgtctttaaacctaagaattctgctgctttcaaattctttcctctgctccttttcctcacctcctgcttctccagcccttccctccatccttctcatccttcaggccctcctctccccttagtccccaccacactgtcactcatgaattgtgacactagcactgtcccatgacctgctacgtaactgttcttttcacagtggccctgctcctgtgagtcggagtgtgtcatttctccacttaaaacactccagtgtctccacctcggtcttgtgaagcttctggagtgtcaggcacttgagcatatgaggggatacctcgttcattgtagggactaagttaatttttgttgacttaatgaatgaaatatgagtgtattaaattgcatcacagataattataaactgtaaaacactgaaaaagttcagaaagattttattttatgcaactagtgtgtatatcaattcatcaattcattcgcacactaccatgcctggcaaaacaacacacatttatctgcttatagttccttggtcagaaatctgggcaagatgtggatagaatctccattctgggcttccaaaagctgtgttttcattttgaatcctccttcgggtttatacagaggtggcagaatgcagtttcttgcagttgtaagactgaggtccctgttcctggctagctgtcaatgtagagaactgggaggggctcaattcccggtgctcaccagcgttctttcctacacagccccttaatttttctttttttttttttttttgagacggagtctggctctgtagcccaggctggggtacagtggcccaatgatctctgctcactacaagttctgcctcctgggttcacaccattctcctgtctcagtcccaagtagctgggactacaggcgcccaccactatgctctaattttttttttcttagtagagacagggtttcaccttgttagccaggattgtctcgacctcctgacctcatgatccgcccgcctcggcctcccaaagtgtcgggattacaggcgtgagccaccgcgcccggcccatagccccttccttttcaaagcccatggtggaggaaacccctcatgctgagtccctttcacactgtaagtctctatgctcatgaagaacccagtcctttcaagggctcacctgattaggacagtccaagcaggataaacccagcataaagtcaactaattgagacccttaattatatctgctaaatcccttcacagcagcacctacattagagttggttgaataactggaggaaggtgaatgaccaggagctgcttgttgaggccatcatagaatcagcctagcaagggttggatcttccttttgtgtttaattgggacacagttggaaagtgaagttcaagtaacgtgatcattgtgaatggtcataaaatacatcctcttcagccatggaaattgtccttactttttaaaactaagttacatgtttaatatcttataattaatttaggccaggtgtggtggctcacatctgtcatcctagcactgtggaaggcagaggaatggagatttgttgactccagaagttcaagatcagcctgggcaaaacctccatctatacaaaaaattagaaaattagccaggcatggtagttcatgatggtagtcgcagctactcaggaggctgaggtcagagagtcccttgaccccaggagttcgacactgcagtgcatggtgatcatgccactgcattccagcctgggtgacagagcaagaccctgtctcaaaaataataatgatgatgatgacacatttagagcaaatgcaatttgatgtgtaataatacatcctctcttgtgaaaatatattatttactgttgcataacaaattacgtaaaacttagctcgaaacaacaaatgtttctcatctcccacagtttccaatcgtcaggaatccaggagaggtttccctgagtgcttcctgctcagggcctctcacaaggttgcactccagttgtcagccaggggccgcagcatctgagggcttcgctggggctgggaattcacaagaaacatggatcagtcacatggctgttggaaaaggcctggttccttgttgtctgttcccagaaggcctcagttctcagtcatgtggaccttcctgcagggctgcttatggcacagcggctggcttccccaagagctcatgatcccagattcagagagagagaaggtggaagccgcagttagttttatgttctacacccagagtcacaaactgttatgttggcactattctataagttagaagttgtattagtccattctcacactgctataaagaaatacctgagactgggtaatttataaaggaaagaggtttaattgacccacagttctgcatgactgaggaggctgccccaggaaacttacagtcatggcagaagtggaagcaaacccgtccttcttcatgtggtgacaggagagagaaatgcacaatgaagcagggaaaagccccttataaaaccatcagatctcatgagtattccctcagcatcaggagaacagcatgggggcaccgtccccatgatccaatcaactcccatgaggtccctcccccaataggtagggattacaatttgcataacaactcaagatgacatttgggtgggaacacagaaccaggccatttcagaagtgcctcattaagtccaagccacactcaagagagggaattaagctgcacctccagaaaggaacagtatcaaaggatttgaatatatgttaaaagcaaaattataactattgtttcaggtttctgaaaatcaaagtcttcttgtatctaattatttttcattaactctttaagcttgtcttttaatttaatttttttttaagttccagggtacatgtgtaggatgtgcaggtttgttacataggtaaatgtgtgccatgttggtttgctgcacctgtcaactcatcacctaggtattaaccctggcatgcattagctatttttgctaatgatcctctcatcaccaccctcccccaacaggccccagtatgtgttgttcctctccctgtgtccatctgttctcattgctcagttcccagttatatatgagaacatgtggtgtttggttttctgttcctgtgtgagtttgctgaggataatggcttccaccttcatccatatccctgcaaaggacttgatctccttcctttttatggctgcataatattccatggtgtatatgtaccatattttctttatccagaccatcattgatgggcatttgggttgattccatgtctttgctattgtgaatagtgctgccatgaatgtaaacatgcatatgtctttataatagaatgatttatattcctttgggtatataccctgcaatgggattgctgggtcaaatggtatttctggttctaaatctttgaggaatcgccacactgtcactgtcttccacaatggattaaccatttacttttccaccaacagtgtaaaagcattcctatttctccgcaaccttgccagcatctgttgtttcttgactatttaataattgccattctgactggcataaaatggtatctcatttgtcgttttgatttgcatttctctaatgatcagtgatgggagctttctttttcatatttttgttggccacatgtatgtcttctttagaagtgtctgttcatgttctttgtccactttttaatgaagttttttttcatgtaaatttgcttaagttctttgtggattctgaatactagacttttgtcagatgaacagatggcaaaatttttctcccattctgtagcatgtctgttcactctgattgatgatagtttcttttgctgtgcagaaaaaaattgcctaatctgattgcttttcatttacaaaaatgagaatctcatacttcagttaacacatctagcttagcatgatgccaataattttggcttgatgctgtagcccgaaatggttagctcgaattgagagacaagttattttcaaaaactggctatccgatgggcattctataacacacttaacatcttttgttgtggtattgaatgagatgtgatagaagtgatttggacaccactatgattctaaatagcactgctattatgtgcttctgaattttttttctttttagcagtcctattctgtttccattcatatttgctattccatcactgggtattccttcctactcttttcatgtttcatttattttgatcagtgatgtgcatttccaattctgtaaaagtttaattcagttgtatgtgtggtaaaatgtaacatagaatctttgcaagatgaaattaccttgcacagcatattgaacattgtattattgaaaatctagagagatgccagtgagccaaggatcgaatgacctatatgtagccaaggccgttttcatggcatcctggtcctctctgtcacatagctccttacaattctctgtggttttcttccaatgtaagtagttcttgttaagaatctttgcaatgaactttgaaattcttttctttttcattgctataaaaaacaggtatattttaatttggaaaactctgtttaaggaattataatcaagtaactaccagggggcccagcactggtgtaaaacacaaaactgtaaaactaccctgggctgctacccagtactcccattgcacaggacagttttactttcccacttgaaaagtgcatctttcagatgccagagtgttgttccaatctgtaaatcttttgcagaactaaaccagtcaagaagtaccatatactccacattccccctcttcttccatgtatctatagaatcatctgggaggtgtgcttcaatccagctagcagtgactcctggactgatggcttttccaggaacactgagagaatgtaaaatacaggaatcctgataatcctgcattctttgagcatccattataatcaagctgatgtttttatccatcatcattgtgtatagttcctttgctgtgattgctcctttctctttggtctcacatttttcattcttttcaccattgctcttttgggttttgtctttggaatccaatacaatctccaaagaatctttagccaacgtgctgccatcctctcttcctgtttcctgccttttttgttgtagccactgtgcttccccctgcctgtccttttcctcaagttttttccagatttcaacttcttcatatcttagtttaaggctttcagagagtcgttcagtttctccagtggcttttttgatgtttgcaggtccaagtattgaatggaagtaatactgctgttgcttgaaatcagttcttttttggataggattataaacagtcatgtttcatatacatgtgtgagctttttttcatatgtttcttggccacgtaaatgtcttcctctgagaagtgtctgtttatatcctttgcccactttttgatggggatgggtttttttcttgtaaatttgtttaagttccttgtaaacaaacatgtgagctctcatcattcttgtttaaacacctaacagtcatcctaaccagtgcaacaaggaccaggcatggtggctcatgcctgtaatccctgcattttgggaggctgaggtgggaggatcacttgagatcaggagtttgagaccagcctgataagtgagacctcatctctaccaaataaaaataattttaaaagaaaatagatcaatggataagaaaggaagaaatgaaagtctttctttgtcaccagcttcattgtatatgtagaaaacactagggaattctgaaaaagtctctggaattaatcattgaatttgcaaaatagttcataaaatatatgtaataagtcgcttagatgaacatgaaaagatagcaaacaatactagtcatcaaagaagtgcaagttaaaaccacaatgagaaaccatcacacatcacctagagcaggtaaagttaaaaagacatatgataagtcttaacactggcaagaatatggaaaaaataggaatgtctgatattgctggtaggaatgcaaaaaatgtggcagccaatttgtaaagtggtatggcagtttcttatacagttacccatctattaccacatggcccagcaattccacaaatacgtatttatccaaaagaaataaaaatgtaagtccacacttgtaagcagttatttatagtggcttcattaataacaagccctaactggaggaatccacatgtctatcaactggagacacagaaaccaatgaataaactgggattccagcaatactcggcagctgctcagcaacaaaaatgaatgaatggcatcatctcaaacatcgttatgctaagggagagaccaaacaaaagactacataacatatgattgcatgtccatgaaattctagaaatgtcactattgcagtgacagaaagcacagcagtggttgaatgaagggaagggggtgagggtgggaggcaaggattaaatagaaaggggcataaagaaagtttttagggaaaagaaactgtcctctacagcgccacaactcaggagtgactgggagggggaagtaaggggagaagaaggtctgagggataaggggcagagagaagggctggggaagcaggaggagaggacgaggagcaggggaaaggactctaaagcagtggaggggcctagtaggaggatctttgcatttggtgtttctctactgggcagtgtggtagttacactataaataattaccaatatccaccaaaaagtgcagctaaaactggtgaatttttttacacgtaaacgccctgataagcaaaaaaaaaaaaaaaaaaaaaaaaaaaaaaaaaaaaaaagggggggggggaggaaagaaggcaaaaataaagacattgttagatcatcaaacccataaaattcatttcctaggggtcctgtactacatgtaattttaaaggacgttctacaggctgaagggaaatgatactagatggtgacccagatacacagaaaggaacaattaacaacagaaatgatgcacatacacatatcacatacacacccattttcttaatttcctgaagatacgtgactgcttgtctaaaacaaaaagtattacactgtattgttgagtttataacgtatattgatgtaacatatacaataataatagcataatgatagtttaaatgaaactacaccatttgaagtgtcctttattttgctggatgcagcttaatattacctgaatttcactgtgaaaagtcaaggaattgggtttcaattcttacaacaataaaaaattaatgtaaagaaatatagctaaaagccactaggagaattaaaaccataagctaaaaaatgtttacttgaaacataagaaagtagcaaaggaggaacagaaacaaaaagatatgagacaaattgaaaacgtatagcaaaatggtagaccaaaatccaaccattataagtgaagaaatgacacgacctgagtcacattagcaggactgctgagcactatggggagaacagacatgggcaggaagtgagggacagtgttagtgccacaattcaggagtgacagggtggcggggactaaaggggaaagagggtgtgagggatgagaggggcagacagaagggctggagaggcaggaggtgaggaaaaggagcagaggaaagaattctaaagcagtagaagagcctggcagggggttctttgcattcggtatttaatacattttgtgtgactgccttaaaactaatgggctccttatgatttttttttaaaaaggggttacaaaaatatcaagtgtccaaataaaatatgcacactgcttagatgtgcatagttccggaaaacgggcagtgctggagcgctggtgaagagcattgggactgcatggagccctcacaactttgaggtgatgactacaggctcccggttgcaatagacagtaacaaaccctgcttctttgtattcaggagatgttctggactcacacagggaaactctggctagagaatgaggataactttaaatgcaacaacccagagtcacagatccatagtctgcgaaagtaaaacaggagctttgagaatttaattgtaatgcagttttgacacaggtctttcacagattggaattctaatcattcagggattaccaatattgtgctacctactgtatcaataaacaaaaaggaaactggtctctatgagaatctctacctggtgctttcagacaaaacttcaccaggtttaaagagaaaactcctgactctacacgtccattcacagggcgagctcactgtctggcatcaagttccccatggtgagtttccctgtacaagagtccaaggggagaggtaagtgtcctttattttgctggatgtagtttaatattacctgaggtaaggtaaggtaaggcaaagagtgggaggcagggagtccagttcagggacggggattccaggaggagaagtgaaggggaaggggctgggcgcagcctgggggtctctccctggtttccacagacagatccttggccaggactcaggcacacagtgtgacaaagatgcttggtgtaggagaagaggaatcaggacgaagtcccaggtcccgggcggggctctcagggtctcaggctccaagggccgtgtctgcactggggaggcgccgcgttgaggattctccactcccctgagtttcacttctcccaacctgcgtcgggtccttcttcctgaatactcatgacgcgtccccaattcccactcccattgggtgtcggattctagagaagccaatcagcgtctccgcagtcccggttctgaagtccccagtcacccacccggactcagattctccccagacgccgagatgcgggtcatggcgccccgaaccctcatcctgctgctctcgggagccctggccctgaccgagacctgggccggtgagtgcggggttgggagggaatcggcctctgcggagaggagcgaggggcccgcccggcgagggcgcaggacccggggagccgcgcagggaggagggtcgggcgggtctcagcccctcctcgcccccaggctcccactccatgaggtatttctacaccgctgtgtcccggcccggccgcggggagccccacttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccagtgagtgaccccggcccggggcgcaggtcacgacccctcctcatcccccacggacggcccgggtcgccccaagtctcccggtctgagatccaccccgaggctgngctgcggaacccgcccagaccctcgaccggagagagccccagtcacctttacccggtttcattttcagtttaggccaaaatccccgcgggttggtcggggcggggcggggctcgggggacggggctgaccgcgggggcggggccagggtctcacatcatccagaggatgtatggctgcgacgtggggcccgacgggcgcctcctccgcgggtatgaccagtacgcctacgacggcaaggattacatcgccctgaacgaggatctgcgctcctggaccgccgcggacacggcggctcagatcacccagcgcaagtgggaggcggcccgtgaggcggagcagctgagagcctacctggagggcctgtgcgtggagtggctccgcagatacctgaagaatgggaaggagacgctgcagcgcgcgggtaccaggggcagtggggagccttccccatctcctgtagatctcccgggatggcctcccacgaggaggggaggaaaatgggatcagcgctagaatatcgccctcccttgaatggagaatgggatgagttttcctgagtttcctctgagggccccctctgctctctaggacaattaagggatgaagtccttgaggaaatggaggggaagacagtccctagaatactgatcaggggtcccctttgaccactttgaccactgcagcagctgtggtcaggctgctgacctttctctcaggccttgttctctgcctcacgctcaatgtgtttgaaggtttgattccagcttttctgagtccttcggcctccactcaggtcaggaccagaagtcgctgttcctccctcagagactagaactttccaatgaataggagattatcccaggtgcctgtgtccaggctggcgtctgggttctgtgcccccttccccaccccaggtgtcctgtccgttctcaggatggtcacatgggcgctgttggagtgtcgcaagagagatacaaagtgtctgaattttctgactcttcccgtcagaacacccaaagacacacgtgacccaccatcccgtctctgaccatgaggccaccctgaggtgctgggccctgggcttctaccctgcggagatcacactgacctggcagtgggatggggaggaccaaactcaggacactgagcttgtggagaccaggccagcaggagatggaaccttccagaagtgggcagctgtggtggtgccttctggagaagagcagagatacacgtgccatgtgcagcacgaggggctgccggagcccctcaccctgagatggggtaaggagggggggtgagtcctgnatgtcttctcagggaaagcagaagtcctcctggagcccttcagccaggtcagggctgaggcttgggggtcagggcccctcaccttcccctcctttcccagagccgtcttcccagcccaccatccccatcgtgggcatcgttgctggcctggctgtcctggctgtcctagctgtcctaggagctgtggtggctgttgtgatgtgtaggaggaagagctcaggtagggaaggggtgaggagtggggtctgggttttcttgttccactgggagtttcaagccccaggtagaagtgtgccccacctcgttactggaagcaccatccacacatgggccatcccagcctgggaccctgtgtgccagcacttactctgttgtgaagcacatgtgacaatgaaggacagatgtatcaccttgatgattatggtgttggggtcctgattccagcattcatgagtcaggggaaggtccctgctaaggacagaccttaggagggcagttgctccagaacccacagctgctttccccgtgtttcctgatcctgccctgggtctgcagtcatagttctggaaacttctcttgggtccaagactaggaggttcccctaagatcgcatggccctgactcctccctgtcccctcacagggcattttcttcccacaggtggaaaaggagggagctgctctcaggctgcgtggtaagtgatggcggtgggcgtgtggaggagctcacccaccccataattcctcttgtcccacatctcctgcgggctctgaccaggtctttttttttgttctaccccagccagcaacagtgcccagggctctgatgagtctctcatcgcttgtaaaggtgagattctggggagctgaagtggtcgggggtggggcagagggaaaaggcctaggtaatggggatcctttgattgggacgtttcgaatgtgtggtgagctgttcagagtgtgatcacttaccatgactgacctgaatttgttcatgactattgtgttctgtagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggacagacagcccacccccgtgtccaccgtgacccctgtccccacactgacctgtgttccctccccgatcatctttcctgttccagagaagtggggctggatgtctccatctctgtctcaacttcatggtgcgctgagctgcaacttcttacttccctaatgaagttaggaacctgaatataaatttgttttctcaaatatttgctatgaagggttgatggattaattaaataagtcaattcctggaagttgagagagcaaataaagacctgagaactttccagaatccgcatgttctctgtgctgagtctgttgcaggtgggggtggggaaggctgtgaggagccgagtgtggacggggcctgtgcctagttgctgttcagttcttcatgggctttatgtagtcagtccttagctgggtcaccttcactgctccattgtccttgtcccttcagtggaaacttgtccagcaggagctgtgaccacagaggctcacacatcgcccagggcagcccctgcacacgggagtccctgtgctttctgagacaaattttcagacccattcagctcctgccctccttatagggctcctcttctgctttggtctcctgccctctctcccttccctgattccagtgatcttcgtgctgactccaatcccaactcatgaatctaaagcagagcctaatttagatttgtatttgtttgtaaaattgggtccatagtctagaattgttccttcctgaagagagaaacctgatcgtgtgctgcagcgtgcagggcggttggtgtgggaggagggataggggagggaggacacacaagcagccctgctgagaaaagtacaggcggcctcggtgtcagtgtgaggggnccttgtgctgcagctgccacaaaacagcacttggcctgaggctatgttaataaagatactggctttagaataggaggtgctctacactgatcattcaactgacctttgttgtcagccagacacaggacagaaaagttctgcatctggggaacaccattgaagtaaaatcagaaaaatatctgagcatatgctccagtggtaagaggcagacgatacatacactataaccacagtaagaaaagaaagtgatggaaggtggtaagtgccatgaggcaggtgatccgggtatgggcagtggggacagggaaggtggctgttggacaggagttgtcaatgtgtgccttgttgcaaagatgacctttgaggaaagatttgagggacatgaggatgtctggggaagttctttctaggcaaggaaacacaacttcgtatagaaaggtatatacgaagttgtgcggccgcgtcgaccaattctcatgtttgacagcttatcatcgaatttctgccattcatccgcttattatcacttattcaggcgtagcaaccaggcgtttaagggcaccaataactgccttaaaaaaattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagattatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgttaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggac

SEQ ID NO: 9 - DNA, 10865 bp, synthetic construct
  source 1..10865: mol_type = other DNA
ccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtcctagggcggccgcataacttcgtatagcatacattatacgaagttatccggaaccgagtacaagcccacggtgcgcctcgccacccgcgacgacgtccccagggccgtccgcaccctcgccgccgcgttcgccgactaccccgccacgcgccacaccgtcgatccggaccgccacatcgagcgcgtgaccgagctgcaagaactcttcctcacgcgcgtcgggctcgacatcggcaaggtgtgggtcgcggacgacggagccgctgtggcggtctggaccacgccggagagcgtcgaagcgggggcggtgttcgccgagatcggcccgcgcatggccgagttgagcggttcccggctggccgcgcagcaacagatggagggcctcctggctccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccgcatgcggcagtggagagggcagaggaagtctgctaacatgcggtgacgtcgaggagaatcctggcccaggcgccgtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggcgacctccagttctccccctggattctggtccctcatatcgggtatggcttccatcagtacctgccctaccctgacgggatgtcgcctttccaggccgccatggtagatggctctggataccaagtccatcgcacaatgcagtttgaagatggtgcctcccttactgtgaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggaactcaagcactccaagaccgagctgaacttcaaggagtggcaaaaggcgtttaccgatgtgatgggcatggacgagctgtacaagtgaattcgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgggagctcataacttcgtatagtatagtatatacgaattatacgcgtatccatactggcgcgccacaacttcgtatagaaaggtatatacgaagttgtgtcgaccaattctcatgtttgacagcttatcatcgaatttctgccattcatccgcttattatcacttattcaggcgtagcaaccaggcgtttaagggcaccaataactgccttaaaaaaattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggac

SEQ ID NO: 10 - DNA, 10664 bp, synthetic construct
  source 1..10664: mol_type = other DNA
ccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtcctagggcggccgcataacttcgtatagcatacattatacgaagttatccggaaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaatggcagtggagagggcagaggaagtctgctaacatgcggtgacgtcgaggagaatcctggcccagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgttgaccgcaagttggacatcacctcccacaacgaggactacaccgtggtggaacagtacgaacgctccgagggccgccactccaccggcggcatggacgagctgtacaagtgaattcgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgggagctcataacttcgtatagcgtgcgttatacgaagttatacgcgtatccatactggcgcgccataacttcgtatagtatagtatatacgaattatgtcgaccaattctcatgtttgacagcttatcatcgaatttctgccattcatccgcttattatcacttattcaggcgtagcaaccaggcgtttaagggcaccaataactgccttaaaaaaattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggac

SEQ ID NO: 11 - DNA, 14161 bp, synthetic construct
  source 1..14161: mol_type = other DNA
ccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtcctagggcggccgcataacttcgtatagcatacattatacgaagttatccggaaccgagtacaagcccacggtgcgcctcgccacccgcgacgacgtccccagggccgtccgcaccctcgccgccgcgttcgccgactaccccgccacgcgccacaccgtcgatccggaccgccacatcgagcgcgtgaccgagctgcaagaactcttcctcacgcgcgtcgggctcgacatcggcaaggtgtgggtcgcggacgacggagccgctgtggcggtctggaccacgccggagagcgtcgaagcgggggcggtgttcgccgagatcggcccgcgcatggccgagttgagcggttcccggctggccgcgcagcaacagatggagggcctcctggctccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccgcatgccccacgctactgcgggtttatatagacggtccccacgggatggggaaaaccaccaccacgcaactgctggtggccctgggttcgcgcgacgatatcgtctatgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggtatgccttatgccgtgaccgacgccgttctggctcctcatatcgggggggaggctggtagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggccctgccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgcgagatgggggaggctaacgagctcggcagtggagagggcagaggaagtctgctaacatgcggtgacgtcgaggagaatcctggcccaagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgacagaaaccaagacaagcgtgtccaggcacatctactttttcagcgccagaggcaggatcgaccccctggtgtacctggacgagtttctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggcagcatcagataccccgcctggaacggcatcatcagccaggaggtgctggactacctgagcagctacatcaacaggcggatcaccggttgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctggctccggcgagggcaggggaagtcttctaacatgcggggacgtggaggaaaatcccggcccagtgagcaagggtgaggaggataacatggcctctctcccagcgacacatgagttacacatctttggctccatcaacggtgtggactttgacatggtgggtcagggcaccggcaatccaaatgatggttatgaggagttaaacctgaagtccaccaagggcgacctccagttctccccctggattctggtccctcatatcgggtatggcttccatcagtacctgccctaccctgacgggatgtcgcctttccaggccgccatggtagatggctctggataccaagtccatcgcacaatgcagtttgaagatggtgcctcccttactgtgaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggaactcaagcactccaagaccgagctgaacttcaaggagtggcaaaaggcgtttaccgatgtgatgggcatggacgagctgtacaagtgaattcgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggctcgagggaagttcctatactctagaaagaataggaacttcggcggacgcgtatagatcaggcgcgccacaacttcgtatagaaaggtatatacgaagttgtgcggccgcgtcgaccaattctcatgtttgacagcttatcatcgaatttctgccattcatccgcttattatcacttattcaggcgtagcaaccaggcgtttaagggcaccaataactgccttaaaaaaattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggac

SEQ ID NO: 12 - DNA, 13971 bp, synthetic construct
  source 1..13971: mol_type = other DNA
ccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtcctagggcggccgcataacttcgtatagcatacattatacgaagttatccggaaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaatgcatgccccacgctactgcgggtttatatagacggtccccacgggatggggaaaaccaccaccacgcaactgctggtggccctgggttcgcgcgacgatatcgtctatgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggtatgccttatgccgtgaccgacgccgttctggctcctcatatcgggggggaggctggtagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggccctgccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgcgagatgggggaggctaacgagctcggcagtggagagggcagaggaagtctgctaacatgcggtgacgtcgaggagaatcctggcccaagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgacagaaaccaagacaagcgtgtccaggcacatctactttttcagcgccagaggcaggatcgaccccctggtgtacctggacgagtttctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggcagcatcagataccccgcctggaacggcatcatcagccaggaggtgctggactacctgagcagctacatcaacaggcggatcaccggttgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctggctccggcgagggcaggggaagtcttctaacatgcggggacgtggaggaaaatcccggcccagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgttgaccgcaagttggacatcacctcccacaacgaggactacaccgtggtggaacagtacgaacgctccgagggccgccactccaccggcggcatggacgagctgtacaagtgaattcgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggctcgagggaagttcctatactctagaaagaataggaacttcggcggacgcgtatagatcaggcgcgccataacttcgtatagtatagtatatacgaattatgcggccgcgtcgaccaattctcatgtttgacagcttatcatcgaatttctgccattcatccgcttattatcacttattcaggcgtagcaaccaggcgtttaagggcaccaataactgccttaaaaaaattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggac

SEQ ID NO: 13 - DNA, 5859 bp, synthetic construct
  source 1..5859: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtgccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacag

SEQ ID NO: 14 - DNA, 113687 bp, synthetic construct
  source 1..113687: mol_type = other DNA
gggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtcctagggcggccgcataacttcgtatagcatacattatacgaagttatccggaaccgagtacaagcccacggtgcgcctcgccacccgcgacgacgtccccagggccgtccgcaccctcgccgccgcgttcgccgactaccccgccacgcgccacaccgtcgatccggaccgccacatcgagcgcgtgaccgagctgcaagaactcttcctcacgcgcgtcgggctcgacatcggcaaggtgtgggtcgcggacgacggagccgctgtggcggtctggaccacgccggagagcgtcgaagcgggggcggtgttcgccgagatcggcccgcgcatggccgagttgagcggttcccggctggccgcgcagcaacagatggagggcctcctggctccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccgcatgccccacgctactgcgggtttatatagacggtccccacgggatggggaaaaccaccaccacgcaactgctggtggccctgggttcgcgcgacgatatcgtctatgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggtatgccttatgccgtgaccgacgccgttctggctcctcatatcgggggggaggctggtagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggccctgccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgcgagatgggggaggctaacgagctcggcagtggagagggcagaggaagtctgctaacatgcggtgacgtcgaggagaatcctggcccaagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgacagaaaccaagacaagcgtgtccaggcacatctactttttcagcgccagaggcaggatcgaccccctggtgtacctggacgagtttctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggcagcatcagataccccgcctggaacggcatcatcagccaggaggtgctggactacctgagcagctacatcaacaggcggatcaccggttgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctggctccggcgagggcaggggaagtcttctaacatgcggggacgtggaggaaaatcccggcccagtgagcaagggtgaggaggataacatggcctctctcccagcgacacatgagttacacatctttggctccatcaacggtgtggactttgacatggtgggtcagggcaccggcaatccaaatgatggttatgaggagttaaacctgaagtccaccaagggcgacctccagttctccccctggattctggtccctcatatcgggtatggcttccatcagtacctgccctaccctgacgggatgtcgcctttccaggccgccatggtagatggctctggataccaagtccatcgcacaatgcagtttgaagatggtgcctcccttactgtgaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggaactcaagcactccaagaccgagctgaacttcaaggagtggcaaaaggcgtttaccgatgtgatgggcatggacgagctgtacaagtgaattcgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggctcgagggaagttcctatactctagaaagaataggaacttcaaaggaagtgcttcagggtgaaggaaaatgatactagatggtgacccagatatacagaaaggaacaattaacaacagaaatgatgcacatacacatgtcacattcacactcattttcttaatttcctgaagacatatggctacttgtttaaaacaaaaagtattacactgtatcattgagcttataatataggttgatgtaatatatacaacaataatagcacaatggtaggttacaagaaactacactgttagaagtgtcctttattttctggatgcagcttaatataacctgaacttcactgtgaaaagtcaaggaatcgggtttccattctttgctattgtgtaaagaaatatagccaaaagccactaggagaattaaaaccataaactaaaaaatgtttatttgacacataagaaagtagtaaaggaggaacagaaacaaaaagatatgagacaaattgaaaacgtatagcaaaattgtagaccaaaacccaaccattgcaagtgaagaaatgacatgacctgagtcacattagcaggactgctgagcactgtggggagaacagatatgggcaggaggtgagggacagtgttagtgccacaattcaggagggaaagggtggcagggactaacgggggtggtgtgaaggatgagaagggcagacagaagggctggagaggcaggaggagaggaaaaggagcaggagaaagaattctaaagcagtggaagagcctggcagagggttctttgcattcggtatttaatacattttgttggacttcctaaaaactaattggctccttatgattaaaaaaaaaagagttacaaaaataccaagtgttcagataaaatatgcacactgcttggatgtgcagagttcaggaaaacaggcagtgcttgagcgctggtgaagagcattgggactgcatggagccctcgcaactttgaggtgatgactacaggttcccggttgcaatagacagtaacaaaccctgcttctttatattcaggagatgttctggactcacacagggaaactcagggtggggaatgaagataattttaaatgcaacaacccagagtcacagatccatagtctgggaaagtaaaacttaggagctttgagagtttaattgtaatgctgttttgacacaggtcttttgcagattggaattctaatcattcagggattaccaatattgtgctacctactgtattaataaacaaaaaggaaactggtctctatgagaatctctgtgtggtggcttcagacaaaacttcgccaggtttagagagaaaacccctgtctctacacctccattcccagggcgagctcactctctggcatcaagttctccgtgatcagcttccctacacaagantccaagaggagaggtaaggagtgagaggcagggagtccagttcagggacagggattccaggaggagaagtgaaggggaagcgggtgggcgcagcctgggggtctctccctggtttccacagacagatccttgtgcaggactcaggcagacagtgtgacaaagaggctggtgtaggagaagagggatcaggacgaagtcccatgtcccggacggggctctcagggtctcaggctccgagggccgcgtctgcattggggaggcgcagcgttggggattccccactcccacgagtttcacttcttctcccaacctatgtcgggtccttcttccaggatactcgtgacgcgtccccatttcccactcccattgggtgtcgggtgtctagagaagccaatcagtgtcgccggggtcccagttctaaagtccccacgcacccacccggactcagagtctcctcagacaccgagatgcgggtcacggcaccccgaaccctcctcctgctgctctggggggccctggccctgaccgagacctgggccggtgagtgcgggtcgggagggaaatggcctctgtggggaggagcgaggggaccgcaggcgggggcgcaggacccggggagccgcgccgggaggagggtctggcgggtctcagcccctcctcgcccccaggctcccactccatgaggtatttctacaccgccatgtcccggcccggccgcggggagccccgcttcatcgcagtgggctacgtggacgacacgcagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccggtgagtgaccccggcccggggcgcaggtcacgactccccatcccccacgtacggcccgggtcgccccgagtctccgggtccgagatccgcctccctgaggccacgggacccgcccagaccctcgaccggcgagagccccaggcgcgtttacccggtttcattttcagttgaggccaaaatccccgcgggttggtcggggcggggcggggctcgggggacggggctgaccgcggggccggggccagggtctcacacttggcagacgatgtatggctgcgacctggggccggacgggcgcctcctccgcgggcataaccagttagcctacgacggcaaggattacatcgccctgaacgaggacctgagctcctggaccgcggcggacaccgcggctcagatcacccagcgcaagtgggaggcggcccgtgaggcggagcagctgagagcctacctggagggcacgtgcgtggagtggctccgcagatacctggagaacgggaaggagacgctgcagcgcgcgggtaccaggggcagtggggagccttccccatctcctataggtcgccggggatggcctcccacgagaagaggaggaaaatgggatcagcgctagaatgtcgccctcccttgaatggagaatggcatgagttttcctgagtttcctctgagggccccctcttctctctaggacaattaagggatgacgtctctgaggaaatggaggggaagacagtccctagaatactgatcaggggtcctctttgacccctgcagcagccttgggaaccgtgacttttcctctcaggccttgttctctgcctcacactcagtgtgtttggggctctgattccagcacttctgagtcactttacctccactcagctcaggagcagaagtccctgttccccgctcagagactcgaactttccaatgaataggagattatcccaggtgcctgcgtccaggctggtgtctgggttctgtgccccttccccaccccaggtgtcctgtccattctcaggctggtcacatgggtggtcctagggtgtcctatgagagatgcaaagcgcctgaattttctgactcttcccatcagaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatggggtaaggagggggatgaggggtcatatctcttctcagggaaagcaggagcccttctggagcccttcagcagggtcagggcccctcgtcttcccctcctttcccagagccatcttcccagtccaccatccccatcgtgggcattgttgctggcctggctgtcctagcagttgtggtcatcggagctgtggtcgctactgtgatgtgtaggaggaagagctcaggtagggaaggggtgaggggtggggtctgggttttcttgtcccactgggggtttcaagccccaggtagaagtgttccctgcctcattactgggaagcagcatccacacaggggctaatgcagcctgggaccctgtgtgccagcacttactcttttgtgcagcacatgtgacaatgaaggacggatgtatcaccttgatggttgtggtgttggggtcctgatttcagcattcatgagtcaggggaaggtccctgctaaggacagaccttaggagggcagttggtccaggacccacacttgctttcctcgtgtttcctgatcctgccttgggtctgtagtcatacttctggaaattccttttgggtccaagacgaggaggttcctctaagatctcatggccctgcttcctcccagtcccctcacaggacattttcttcccacaggtggaaaaggagggagctactctcaggctgcgtgtaagtggtgggggtgggagtgtggaggagctcacccaccccataattcctcctgtcccacgtctcctgcgggctctgaccaggtcctgtttttgttctactccagccagcgacagtgcccagggctctgatgtgtctctcacagcttgaaaaggtgagattcttggggtctagagtgggcgggggggggtggggtggggagggggcagaggggaaaggcctgggtaatggagattctttgattgggatgtttcgcgtgtgtggtgggctgtttagagtgtcatcacttaccatgactaaccagaatttgttcatgactgttgttttctgtagcctgagacagctgtcttgtgagggactgagatgcaggatttcttcactcctcccctttgtgacttcaagagcctctggcatctctttctgcaaaggcacctgaatgtgtctgcgtccctgttagcataatgtgaggaggtggagagacagcccacccccgtgtccactgtgacccctgttcccatgctgacctgtgtttcctccccagtcatctttcctgttccagaggggtggggctggatgtctccatctctgtctcaactttatgtgcactgagctgcaacttcttacttccctactgaaaataagaatctgaatataaatttgttttctcaaatatttgctatgagaggttgatggattaattaaataagtcaattcctggaatgtgagagagcaaataaagacctgagaactttccagaatctgcatgttcgctgtgctgagtctgttgcaggtggggtgtggagaaggctgtggggggccgagtgtggacagggcctgtgcccagttgttgttgagcccatcatgggctctatgtggttagtcctcagctgggtcaccttcactgccccattgtccttgtcccttcagcggaaacttgtccagtgggagctgtgaccacagaggctcacacatcgcccagggtggcccctgcacacgggggtctctgtgcattctgagacaaattttcagagccattcacctcctgccctgcttctagagctccttttctgctctgctctcctgccctctctccctgccctggttctagtgatcttggtgctgaatccaatcccaactcctgaatctgtaaagcagagtctaatttagacttacatttgtctgtgaaattggacccatcatcaaggactgttctttcctgaagggagaacctgattgtgtgctgcagtgtgctggggcagggggtgcggggagggggttgctgtagaaagagggatggggagggatggcctgaggctatgttaataaagatactgccttcaaaatagggaggtgctctacagtgatcattcattcaactgacctttgtcattggccagacataggacagaatggttctgcatctggggaacaccactgaagtaaaatcagaaaaatctctggccttttgtagcatatgttccagtgggaagaggcagacgatagatacactataaccagagtaaggaaggaaagtgctagaaggtggtaagtgctgtgaggcaggtgatccaggatgtgggcagtggggacagggaaggtggctgttgtgctgggtagtcagtgtgtgccttgttgcaaaggtgacttttgaggaaagatttgagagacatgaggatgtctggggaagttctttccaggcagaggaagctccagtccaaatgcactatggcaggaaggtgtttgtgttcccagaagagcaaggaggccaggagggctggacggagagaaactgaggtgaggtcagaggtgcggccagaacaggtgggcttgaggggagtggggttggatctggcctttgctctgagtgggatggggagttagaggacagttttgagcagaagagagccatgatatgacttctgttttaaaaggatctctctgacggctgtgctgagaacagaattgagaggcaagggacgagggaggcagaagggaaaacagtaggaatcaagtgcagtattccaggctggagatgtcggttaccttgactggggcgtgagcaggggaaatagtgggacgtgaggggattctggatgcatttgaagatggactcacagcatttgccaatggattgtatctgtgatgtgagaaagacgaatcaaggacacccatagttgtaaaatgagtgagtagaaggatggagctgctgtcagtggagatggggagactctggcaggagcgtcctgaagaggggacatcacaggcactcagtggaggagatgtctactaggagggcgggtgggggagctgggtggaatttggacagacaactccagagtttaggggaaaggactgggctggagaaatagatttaggaggtcacaccatatatatgagacttaaaacctcaagcatggatgaggcacaaagggagtgactgactatggaaaagaatgagcacaaggactgaaccctggacctccagttctaaggggtgtgatcagatcacacccagagcagactgcacagttctggccccatgtctagaggacactcagacaaggaaaccccatgtgcaccaggatcatctggatgtggtgctgagatcctggaagtctggagttgagcaagagattctggatttatgacaaggctggagctcatgttgctggtctccagatcacacttggagtagcaagaacaccaggatcccacacgtctgagcatcagcctcacctctagggcttgtcatataaatgattccttggtcttgtgcataattctctgagacaagggttctggggagtggcctgtgtattttctaagtccccaccagcaatcctattgctcagacagattgggagccactgagatcagtgatcagagagtgcccagggtgggtgggtggggtgggttttcaaaccctgttgaaaagaggatttttctcacagaaagaaaagggaggatgtatatcatcagttatgagaggtgatattctctgttgttctctccaccatggggtagaggccaggtagacaattcaggatgtggctctcgcacaaagaacacctctgaatgccgctctctgacactcgcccgcagactcatttctcactcacttcttggagaaaactatggaaaccaaatttctgtaatgtacacataaagttgtatattggtattgggggctagttttattgtggggaaggccacagaaccaggctggaaactacacatccaagaacagaattcacagcccaccctggatcaggtccctcctaggaacaactgcccctgctgctgagcacagacagcactgctcacacctctgacatcctggtgctggacactggaccctgaggctaggacagatgtcactgctgcacctggaaattggacatcactactgtcactcagccatctttactaaaatgctttctgcacagtcctagcctctctgtcacctcattctggctgagtctggcagtgatataggagttaagaagaaattatttagacaggtagtgagggtacgcaaggcttttaatggaaagcaggctccaaattattttcttttctaacaaggagcagcctgtaatatcaagcttcagacatagacaagtaagctggaagtttgcacgggtgaatgccagcagctgtccaataggaataggacgcctgggactgggcatgttcactatggcggctccatcttcccttctctttgccaggcacgtttatagtaaggagcagacaacatgtcaccggccaagtggaaagcccatttgcataataggatgagggtggggtgaacagccttccacacgcactatgtaaatatcacacctggtacaaccaacctgtgggccctacataaatcagacaccatctcctcaagcctgcctgtaaaatccgatgcactccattccaggctggaattcccttttgggtgcccctctctctcacaagaaggagctgtttccctttgtctttcttttgcctattaaaccctttgatcctaactcactcctcgtgtgtcagtgtccttaatcttcttggcgtgagatgacgaacacttagtatttacctgagacaatgacacctcttcagcaggtggtcatcagactggtggagcttaaaattcatgtccatgatcaactgcaggggtgactggagtgttgttgtcctcttccagggagggaggtggtgtctgtctcctctcaacattttacatgttgtaaaccaaaaataaacatcaaagccaccccttcccaatcattttaatggacaccctcagccagggtgctcaaaagttaacctgaaagagtggctcaggccaccatgggaagcaggtattgaacatgtcttattatgccctcttccctcttggaattcaggaaaagttgaccagcatttaccatcaacacagaccttcagtctgatcagaaacatttacaatctattctctttgaagcctggcacctggaggcgtcatctgtatgagaattttggactccacaaccttttatcataacccagacattcctttctattgataataacaaccaattgccaatcagaaaaattaaaaatctacttataacccagaagcactaccccgcaacccttgcttgcttcaaattgttccaactttctggaccgaaccaatgtatagcttaaatgtatttgattgatgtctcatatcttccttaaatgtgtaaaaccatgtatttgattgatgtctcatatcttccttaaatgtgtaaaaccaacctgtgcctcaaccaccttgagcacatgttctcggtctcctgagggctgtgtcatgggctgtggtcactcatttttggctcagaataaatctctttaaatatgttactgcgtttgactcttttcattgacagtgtggaattttgatgtagtaagcgggttcaagtgctggagtgtgaggggtggggaaaagtgacaaattttaattcttggagcagtgatccaggacgagaactttatctagtacccagtaggcacgaagagtggctgaatgaaccagtgactaataaataacatatttccacccattcccttgaggataggtttacatcaagtttttgtctatcccagtttatagtctagattattggagtggcattctgtaatgatgaaatgattattcaaacctcttccctcagcatgactcttactaggattaaaactcccaggtttgtaagatatcagaggttctggttccacacgatgttttatattaatatttatattttcttgtgtgtggaaatatttctggggagagaatctctaacatttattagtttttatgtgacaccctcaaaggcctaaaaaatcctaacttctagattaaaggtaggaccactccacgtctagacaatgaacagctggtagcaacctgtgtttagggacatgaaacaaatgcttacatttgtttatcttttcctctgggaccaaagaaggtagaggtttacttgcttactcttactataagtttctctctgtgggatggtgatggggttttcttccacccaaataactttctgattctccaacagcagctgggagtcccacaatttgactcaattctgacactaactacctggagttcgcctcagactctacaggtttaccagctcagtcctacaagtctgtcctcacttcagaagcaagtcacaagtatcgggtcccaggttacctgcacttctgtctaacatggctgcaaagtcaggggtttctcacctcccaaggattgagaattcttaaactaactcaaagaactcaggaagttgctatagccgtgtaaaccaaaaaacgtctgagacaggtctcagtcagtttagaagtttattttgccaagctgaagacgcaccttggaaacagagacacaaatcattgtagcatctgtggcccatgctctttcctaacaggcttttgaggacttcaatatgtacaggggaaagagcagccaggagaggaaaaaaatagtcaactatgcattcatctcatgctcagtaaatctgcattttaaataagataaagtaaacattgggtggaaaagtcaaatatgcgtttgactctgggtgggtggagggatgatttctagtcttgtctttgtcctgtacctgtgaagataagctgttaatttacattgtcaggggaaaattcaacaaaactgttttagggtaaacacgttggggcccacaaggaattatcttgtgagcagtttgtgagggaggccacctggggagatatgtggtcttctatctttgcagttatttgtttaggaacgaaagaaaggcagtttttccatcagtctccaaacttaatttttccttttggtatagtgaatttggagtcctgaaattttattttcttttcacactcagtattgcagttttttattgttatagcttaaatccccacctttttggcacctgggaccggtttcatggaagacagtttttccatggactagtggaggtggtggagggcagagggaaatggtttcggatgaaactgttgtacctcagatgatcaggcattagcgcttcataaggagcatgcaacctagatctctcgcatgcagagttcacaatagggttcgtgcttctatgagaatctaattccgccactgatctgacaggaggcggaactctggtgtcatgctcatgctctctcacccactgatcacctcctgctgtgcagtctggttcctaccaggccaggaacctgtaccagtccgcagcccgggggttaaggacccctggtttaaattatgccactagagcagaactcctgagactggataacttataacaaacagaaatttgtctcgttcatggtgtcgaggatgagaattcccagatcaagggactctatctggtagaaaacaaaaggacaagagagggtgagaggtggggggaaaaggaagtcaaactcacagcctcaggtccttttatcatgagccttagtccatgaatgaaggcagagctctgatggtccaatcacctctcaaaagtcccatttcttaaaaatgttgcatttgggatgaaatatccaacagatgcttttttggggaaacattcaaaccatagcagttataaaggataccaaggagcagccagatgaagaggtgcacagggtgaggcctggaagggctctgagcacaggagcctctgtccccatggagatggggctcaccatcctcccagcatgtggacacacagatgtgctctccgaaacccaatctccagaggtttctatggaaatttcattgtggaggcaagactaattaaatcactggtcattgtgtgactgaactcaacctcgaacctctctttccttcccagactttagggatggggttaacagttccaatcctctaatcacaacttcgtgtttctggcaaccagcccccatcctgaagctatctaggagcccctcaccccatagtcatctcattgccatattgaactcatgtttatcactgaggagattgcaaatgttttagaacctggttgtcaggaaacagggaaaagaccaaatatttttattggaccacagatcacaccctggttttgaccacattatctctttttataatatttattattttattttagagataatctcttgctctgttgcccagggtagaatgcagtggcacaatcatagctcactctaacctcgaattcctggactcaagtgatccttctgcctcagcctcctgagtagatagcattacaggtgcacaccaacatgcccaactaatttttaaatattttgtaggaatgggtctcactattctgcccagtctggtctcaaactcctggacttaaacaatgttcctaccattgcctcccaaactgatggaattataagtgtgagccactgtgcctggccaatcacagatctcctacaccagaagaatcataacagtgaaaagatcctggcacattaccagaatcctctttggtcattaacaatgatcccagtgcaatccataaacaggaatagtttcagcaacatatggctgcaccctttcaggcgtctggtaaaattgagctaagagacaatatcatctcttgcttatacctttttcaagtagttcatgtaacattggattttcctcattacataacccatttattcattcatttaccctcagctactatttctccttcccttcatttatactaaacatttataattttggaaggaacactagcttctgctgctgtgctggctgagactgcagggaccaatagagttctagcaagtgtctcccgtcatccattccagttcatagagggtagggttatacaggtagagaactggttggctatctgatcccaggcaatatagctgcattcagtgttagccccaactttgccagatgcagtgaaggcacaacctacttctacaggcccttaggaattattacataatggacataaaatatatttacagtttcttgcttaggaataattcctgtttctgaactctatttgcatccctagtcctgggaccactgcatcaggtatgagaaaagcaagttgaggtgaattacagtcatcattccagtgtcctcccaccttggggaggaatatatgtagatcataccagcaccttcccctgatccaccaggaaaagagaggatactctcatgtcaagtgtgagcacactcatgaaggtgtataagcccaccctttgtgcttgtttccccactctttcctttacacacccaattgactattttaattctctatggaactcttcctctgaggaggatatctctctgtgaccattgttggacattttgagcaggtgcattttctggtgtaaagaaatatgactcggtggtccacgtggttgcagtacctcccattcaagttcttttgtaacactcgagcatttgcatctgagataaagcagggacctcttttagaagcctgccggggcctcccaatctggaaataaagaaaaaccttcagttccttcaagagaaattccaggcacctagccagccctaaaaagtaagtcggtgaccttataagcaagaaggaatgatagcttgaaacaatagccaaggaagttagagtcacaagatgtttcattctctatagaaactaaaaataacatcttaacatatgtccctgagttgtttttcagaaacatggattccctcctaatagatccactgacagggagaccttataaaggggaaactgagaactgaactctgaccgccgttctttgttctaaatttcttcctgagggacctggagagagtcaagcccacaggccaggcctgaacattcctctctactgcccccaactgtgtaaggaagctgtgtaaagaagctttgcttccttatctatcacaaatcagagaatctttaaatctacctaagacctgtaagctccctcatcaggatattctaccttttaaggccaaagcagtgtaacttgtatgcattgatttatgatgttgcctgtaactctgctttcccgaaatttgcccctgcctttaaaaattcttgcttgtgagccatcaaaaangttgggtcttaagcgtgagctgcctgattctccttgtttggcgccctacacataaatgccctcctttgtcccactacaatctcagtgtggatgtttgcttttactgccccaggtgagtggaccccagttcagtttggtaacacacctaccaccagctgaacaaagcccactccagggtgaggatctattcctgccaagacccgtttgctgcttcctggggctactggtatcagtctaacttgccagctatgtattttcaaggccctcccataaaagaatctgccacatagccatatgctatctctctttttttttgttaaagaaaggacttcttatgtgtattttgtgcctgtgatgtgtggaatgcaagaggactatgtcttgattccacctatctctgcattgctacagccccaatgtctacttatttcatggactcaggtgatcaccacaagcaaacctggattttttgttgttgttgtttttgagatgtggtcttgatatgtttccctggctggcctcaaatgttgctcaggttaaactctaactcctggactcaagatatccttctgcctcagccgctctcatagctggaattacaagtgcacctggcaaacacttggagataaattcttgttgatttcaatcactttccaaacctgaaagggagttattctgaaggacactgaacagcactgaggggacggtgctaaaccattcatgagaaactgacctcgtgatccaaccacctccctccctccaggccccacctataatgctggggatcacatctcgacatgagatctgcacaagaacaacatccaatctatatcaggcatcaacagatcttacattaatgaacccctcaaattaccatagtgatttctgtgggacttggctcatatgggcattcttttcataggcaagtttttcattgctttcctgattaattatacggccaggccattagtcactccctgtaagtcagtaaaaactaaaacgtaggagctttatcattgttcaattcttccaccactgctaagaaaacaccatgcaattaagtccacaacagcttttctgtttttaccttctttgatcaaagaggtggacttccaaacagtatgttgtccattcatcttgcaaatgctgtacacaaaccaaccagctgttagtgggtcagttgactgatactgcagttcaaatcccagcgctgtctgctacagagttccatcttgctcaagagaggtcagtgttttgtttaatttacaccttcagtgaattgaatgaagcccactcacattatagacattaatctactttattcaaagtctggctatttatgtgttaatctcatccaacaaacatcctcacaaatcatccagaataatgcttgaccacctctccaggcacgatgctttacccaagatgaccaataaatttttttttattattatactttaagttctagggtacatgtgtacaacgtgcaggtttgttacatatgtatacatgtgccatgttggtctgctgcacctgttaactcattatttacattaggtatttctcctagtactatccctccccactgcccccaccccacgacaggccccggtgtgtgatgttcccccctgtgtccaagtgttctcattgttcaattcccacctatgagtgagaacatgcggtgtttggttttctgtccttgtgatagtttgaacaatgagaacacttggacacttgtgatagtttgctcagaatgatggtttccagcttcatccacgtccctgcaaaggacatgaactcatcctttttatggctgcatagtattccatggtgtatatgtgacacattttctcccagcacctgttgtttcctgactttttaatgatcgccattcttactggtgtgagatgggataccattgtggttttgatttgcatttctctgatggccagtgatgatgagcattttttcatgtgtctgttggctgcataaatgtcttctttggagaagtgtctgttcatatcctttgcccactttttgatggggttgtttgattttttcttgtaaatttgtttgagttccttgtagattctggatattagccctttgttagatgggtagattgtaaatattttctcccattttgtaggttgcctgttcactgtgatggtagtttcttttgcagtgcagaagctctttagtttaattagatcccatttgtcaattttggtttttgatgccattgcttttggtgttttagtcatgaagtccttgcccatgcctatggcctgaaaggtattgcctaggttttcttctagggtttttatggttttagggatgatcagtaaaattaattatgacattcttcaatataagaatactctgaggaaattagcctgtgatctaataaaacaaggagcaaacaactgtttctaaaaatttataacaaatagtcaaaaagccctcgctagagattgtttctcacattcactctataattctgggtaaaaaaaaaagcacgtatttgttgaaaaccacagaagaagagaaaattgaaaaagcagaaagtgaaaaaaataagaataataataaagatgacagcagttatttcattggaaacaatgcaaatgaggagacagatgagcaacatctttagagtcctaaaaggaaaatgctgtgaaactagattctattcccagaaaataatacctttttcaaatatatatgtatagttttaattgacaaagtataatacattcatgagatattgtttgatgtattgatatatgtgtaccctatgtaatgattaaattaagctaacacaccttgaatatatctatcaaaaaataaagggattttaggccaggcatggtggctcatgtctgtcatcccaacactttgggaggccaaggtaggtagatcacgaagtcaggagtcaaaaccagcctgaccaacatggtgaaacctcgtctctactaaaaatacaaaaattagctaggcatggtggcatgctcctgtaattcccagctactcaggaggctgaggcaggagaatcacttgaacatgggaggcggaggttgcagtaggccaatatcacaccattgcactccagcctgggcagtagagtgagactctgtctcaaaaataaacaaacaaacaaacaaacaaacaaacaaatggattttagacatacagggagtgaaaaaataaccagcagacctaaactataataaatgtttcagtcctataggcaaaaagaaaattaagggactagttggcacagcatttaacgatgaagagacagcttcatcgccccctcccactgagacagagaacacagccccagggcctgggaattcctcaacccaatccaccaccttgggcacctgtgcactcctcctgggtacctgaggttggatgcaatcaccttagctatcacctacctgcaaacaccacctatgggcatcccacccagcctatcgcatctgctgctaacaatgcacactgcttgggacccagagaagcagcccacgactgccactgccaagctggttgcccaggagctgaagtccccagcacctaccgagtcaaccactgccactactgccatccaagcaagccacctggaggtccaggaatcagcccaccagtaaccaccaatacaaatgccagtatataccaccttggggcacaaagataggaacactcagcacaccactgccaccactggagcctgaagactgtctcatgtggcatcccagtccccagcacattttcaccacagcctcactaataaccacaccctaatgcacagaagaaatcacagataccattaacactgtctacagccaaagaaattatacagagaccatagtactctactatgtactacacccagaatcaaatccaaagtaccccaggcaaccaacaccatagactcatcttcaggaaaatgctctccactatgaaacaaaattcaaaaataggaagaactgactgttatatcagatgcaaaaatattaatgttaggacacagaaacatgaaaaagcaaaaaagaaaaaaaaagccgcaaaggaagacaatagatcttaatcaaaaagaaattttcaagattctagataaaggactcaaaacatttattttacagaagttcagtgagatacaaaagaattctgaaaaaaaaaatacaaagaaaacataaaaataattcaggatacaaataagaagtttacaaaagatatagatttaaaaaaaaacaacagaaattctggaattaaagaatacatgacatgaaattccaaatacatttgaaagcttcaacaatatactaggtcaggcagaaggaagaattccaaaacttgaagatatacattgtgaaataattcaataagacaaaaacacacacaaaaaaagaatgagcaaagcctttgtgacatttggtacaatataaacaaccaaatattcaaattattggtatcctcaagagcgaagagacaaagaacagattagaaaacctatttaatgaaagaatagatgaataattcccaactctagcaagtgatttagacattcagatacaaaatattcaacaattctgagaaaaatacaatgcaaaaagttcctctccatggcatataatcagactttctatagtataaggtaaaatgcaaatcctaaaaatagcaagaggaaagtgtttagtcacctataaaggaaaccccatcagactaacagcagatttctttttcttttttctttcttttttttttttttgagacggagtctcgctgtgctgccaggctggagtgcagtggcgtgatctcggttcactgcaacctctgccccctgggttcaggtgattctcctccctcagcctcccaagtagctgggatgacaggcacatgccaccacacacagctaatttttttttttttgtatttttagtagagacaggatttcaccaagttggccaggatggtctcaatctcttgacatcatgatccgcccacctcagcctcccaaagtgctgggattacaggtgtgagccactgtgcccagctctaacagcagatttcttngcagaaatcttacaggccagaagagaataggatagcataatcaaagtcctgaaagaaaagaactgccaagccaagagtaataaaccaaacataaattattcttcataattgaaaaagaaattatctttccaagacaagcaaatgctgaaaggcttcattaccactagacagggcctacaggaaatgctcaagaaagtcctaaacctggaattgaaaggacaacattttcaacatttccacattatcaccatgagaacatacacaagtataaaactcactggtaaagcaatcacacaaaagaggaagagaaagttctcagatggtcctactacagaaatccaccaaagcataatgacaagcaataagagaaaaagacaataacaaaagacatataaaacaaccagagaacaacaatgtgaaaggaacaaagctgttgggaacaggaccccaaaaatctggccataaactggccccaaaactggccataaacaaaatctctgcagcactgtgacatgttaatgatggccatgacgcccacactggaaggttgtgggtttaccagaatgagggcaaggaacacctggcccactcagggcagaaaactgcttcaagttgttcttcaaccaccaacaatagcatgagcgatctgtgccttaaagacatgctcctgctgcagataactagccaaacctatccttttattttggcccatccctttgtttcccataaggaatacttttagttaatctataatctatagaaacaatgcttatcactggcttgagttaataaatacgggggtaaatctctattcgaggctgtcagctctgaaggctgggagacccctgatttcccacttcacacctgtatatttctgtgtgtgtgtctttaatttctctagcgccgctgggttagggtctccccagctgagttggtctcagcacaaagcctcacatattaataaaaatctggaatgtaaacagattaaattctccacttataagatgtagaatggctgaatgggtagaaaacatgatctaactacattctgcttacaaaaaaactcactggtaataccacctcaccacaatcatagagacagaaggtaaaagaatggcaacaggttatctatcgaaaccaaaagtgagcaggagtagctatacttatagcagataaaataaactgtaagtcaagaacaatttttttaaaagataaggaaggccatcacataatcataaagagatccatccaagaagagaatagaacaattctaaatatatatgcactaaacattggagcanctagattcataaaacaaatattactacatctaacaagagagatagactccaatacaataatagtgggggacttcaacatcccaacctcagcataagacagatcacctacacagaaaatcaagaaagaaacattggatttaaactgaattttacaccaaataaagctaacagatatttacagagcattctgtctaacactacagaatatacactcttttcatgaacacatggaacattttccaggagagagcctatgttaagccacaaaacaagtctcaacaaattttcaaaaattgaaatcatattaaatatcttctctggccacaatggaataaaactagaaatatacaccaagagaaatgttggcaacaagaccaatacatggaaattaaacaacctgctcctgaataaccattgagtcaacagagaaattaagatgtaaatcaaaaagtttcttgaaataaatgaaaatggaaacacaacataccaaaacctgtgcgatacagctaaacagggctaaaagggaggtttatagcaataaatacccacctaagaaaagtagaaacattacaaattaacaatataacaatgaacctcatggaaccagaaaagcaagaaaaaaaatatacaaaattagcagaaaagaaataataaagatcatagtagaaataaagacttttaaaaaccatgtaaatgatcaacaaagtgaaaagtttgttcttcaaaaacataaacaaaattgataaaccactagctaaacaaacccagaaaaaaagagagaaaatttattaagaataaaagttcctcttcaaagttttcctttttggtttagaatgataagtcttaagaagatagttcctcttaaaagcttcctttaagtttccctcgctcttcatactaacaaatcttctattttactaatgactctttattgtgccccaaaggagttgttacatacagtaaanaaataaacacattctatgttcttgtacttgaaccaaggcctaactcctttgttccctagtctgcctggatctgcctggacacgcccagacatgtcacagcttgcagcctatgctctttccttatttggaaatattattgtcttcctggtttcccataagcgaccccctcctttccttggttccccattgcgcctttaccttatttgggaaagtttaagtttttagccaactgggatcagtttagattgtgtggtccagttccagccaatggagacaggacacaatagtagaaacaaattgcgttaggaataaaaacccctgctttcctttgttctggttgctctcgtggcaatcaggcttacaggtagcagccttctgcagaagtaaaaattgccttgctgagaaatcctttgatcaagtgttcatttcctttgtgactccaagctttatttctaactattttatgggggctcttccaggattcccattctcctatgggaaggggtctttggtcaccccatctaggggaggtgtaccccactgcagtggccccagggacgggagattgagactacccattgtgatgaataaattcaggctctcagcaatgtggaaggagaaggcccacaagtaccacgatgaccagctaactgtgcagagaccaaggtaaaaaaagtcacaagggcaacagtatttccttggtggtcgggatattctggaggttaaagggtgtgaaaggtaacaagcactactgctgtgcagagtgagtgagttcaatctgtggttccgtggtcacctcatgtggcttatgatggccctcttgtagggatcctgacattggggttcatacagtcctgctgatgctaaggggagcctaatttcctctgggaaagcagccagagtggatgaagcaaaaggagaagattgcaagaaacctccagggtgggggttaaacctccagggaaggataggcaagaaatctctagttcgagggactgagcctaaccagtattcaacatgggaaataccctgagtaagacagaaggtaaaaaggaaaaggcaaacagtgaaatcacctctgatagtcctttaggactcatgttaaaatattggaaggatacaagaggactaaacataagaaaaagcaacagatgataaagtattattgtttcatttggaccaaggaacctattctaaagccttcagttttctggccaaaattcgggtcaaatgaggcttggacttgccaacttttaatagaatgtgtaaatgataagagtcctgtttcccaggaggaaatacattatgctctgtgttggcagcaagggcccatcctcctttgtcatctaaaaaccaaaggagacaagccagaaactacttctcatgaaattaataccccttactccaagaaccccaccaacacgtgggacgccttagaccatcttcccccaacaaaacttcttccccccacccagtagaccaacatcccccacctcccccttcagtagaaataacagtcccagacctttcccctactcagacattttttccctccttttaaccctgattcttggggtcatccccagcttgaatgccctccctcaggaagacttcaacgtgagttagaacaatgtaaaaatgatatccaaaacttcccttttcccccttcctctaggggtttggctacaaacctcttcccactgagggaagtgcctctaggaggagcaggcattggctttttaaatgctcccctgaccagttcggaagtccagagcttgaagagggaactcaagccactcttagatgatccctatggagtggcagatcaaattgatcaatttttagcaccccagttgtatactgtggctgagtttatgtccatcataggcatccttttttcaggggaagaaaggagcatgatgcatagggccgctatgaccatttgggagtgtgagcatcctcccggtcaaaacgtccctgcagccaaacagaaatttcctgctcaagatccccagtgggataacaacaacatagctcataggaggaatatgagagacctgcaagaaatgataataaaaggaattagataatcagtaccctgcacccagaatatgaccaaagcttttaatatacagcaaggaaaagaagaggggcctatggaatattttaataggcttaaggaacaaatgaggaaatatgctggtttagatggaggagacccacttggacaaggaatgttaaaagcttcactttgtcacaaatagctggccagacgttaccaagaaattacaaaagatagaaaattgaaaggactgtcccataaaggaactcttaagggaggcccaaaaggtgtatgtacgaagggatgaagaaaagcaaaagcacaaggcaaagattatgctgttcttaaagcagggaactccccaatagacagctcaaagaaatatcgccggtaaatcttttaggtacccgactgccagactctataatggaggtaaaggaatcaaaccagggaacaagggaataaaaagaggaatagggcagaataaatgcttcaaatgtgggaaggaaggtcactttaaagagaatgccctgaatggaaaaaagaaaaagaaatcacccacttatgatctttgaggaggaatggggggccaggggctctactctttttacctcgagtcccaccaagactccttgataaatttagaggtgggacccagacctttttaattgactcaggagctgctgattcctcagtttgttatcttccccctggtgtaacttggtcacaagaagaactttttattttgggagcaaaaggggaggggtttaaagcaaaagtcttagaagaaacaaaaattaaatatcagaaccactcagtaaatattaaacttctgttaatcctggaggcaggaacaaacctattaggaagagatttaatgctaaaattaaacctaggcctttgtgttaatcaaggaaatctcctcccctccttaaatttgcttgctactctaaatgagggatacatccatccagatgtatggtcaaaggaaggaaactgaggaaagttacaaatttccccaatccaagttaaattgaaaaacctggggcaagaggtaaaaagaaaacaatatccaattcccttagaagctaagatcaatttaaagcccacaatagaaagccttctccatggtggactcattgaaccctgtatgtctccttataacactcccatactgcctgtgaaaaagccagaggggtcatatcggctagtgcaagatcttcgatctattaatcaaatagttcaaacaactcactctgtcgatcctaatccttatgctattattagtaaaattccttacaaccatgagtggttcacagtaatagatctaaaggatgccttctgggcttgcccactagcaaagatagactttgaatgggaggaccctcactctggttgaaaataacagtatcgatggacagttttgccccagggatttacagaatctccaaatttgtttggtcaaatactagaacagatcatagaaaagttttacaaaccaccatatatatgcctgctccaatacatggataatatccttatttcaggggaggatagacaaaaagtagaaggattttcaataggttttttaaaaataatttgcaactggaggggttatgagtgtcaaaaaagcaaacttcggtttgtagaacctgaagtcaagtatttaggggacttaatcagtaagggcaaatggagaattggatttgaatggattgaaggcatcatatccttcatatgatatcctgagacaaagaaggaacttagaaagtttttagggttagtagggtattgttgcttatagagagactcttatgctttagtaaccaaacctctacataaaaaaaacttacacaggatgaattggacccccttatatggttttacaagaaatacagcagataaaaaaattaaaagagttactagtgacagcccatgttctagccttaccctctttagaattgccctttcatctttttgttaatgtaggtaatgaagtagccttaggggtacttactcaaacacacggaggccattggcaacccatagccttcttgtcaaaaattcttcacccagtagcccatggatggcccgagtgtgtcccatcagtggcagcaacagccttgctcatagaggagagcagaaaattaacctttggggaaatcttatcattagtactcctcaacaagtgagaacaattctcaatcagaaggcaggaaggtggcttattgattcaagaattttaaagtatgaagccatcttgttagaaagagatgatctaatcttaaccactgatgactcaattaatccagctgctttcttaacaggaaaccccaacccacagacccctgatctttgcccagagcacagatgtttagatttaattagttatcaaacagaagttagacctgatctaaatgaaacaccctttcatactgggaaacacctgtttgtagatggttcctcccgagtaatcaaaggaaaaaggcataacgagtattcaacgatagatggagataccctcacagaaatagagtctgaaaggttaccaaatgactggtctgcacaaacatgtgagttgttcacattaaatcaggccttaaaatttctgcaaatcaagaagcaacaatctatacagactccaggtatgcctttggagtagtccatacttttgaaaaaatttggactgagcggggcctcattaacagcaaaggtcaaattttagaccatagggacctaataatacaagtactagaaaatttacaattgccaaaagaaatagctgttgtacatgtcccacgacatcaaaggaatcctttatttgaaggccagggaaataatctcactgaccaaatagccaagcaagctgcctcttctccagcagaacccatttttcgactaaccccttgtcttccatcttcagctgcactccctatcttctctcaagcagatcaggaaaaactgaaaaaaataggagctgaagaaaactcaaaaggaaagtgggtattaccagatggaagggaaatgttacccaaactttatgagggaaatattgtcacagcttcatcaaggatgtcactggggtcctcaaaccatgtgtgatgcagtccttagggtttatgggtgcataagaatatacaccctcgccaagcaaatggtggaaagttatatactgtgcagaaaatctaataagcagaccctcaaaagacaacctcccggagggagaaatcctgggttaaggcgatttcaaagtgtccaagttgactctactgaaatgcccccagtaggccgccttaagtatttaccggtaatagtagatcatcttacccactgggtagaagccatcccctttccaagttcaacagccagtaatgtggtcaaagctctgttagaacatatcatacccaggtttggaataatagagaacattgattcagataatgggacccaatttaccgtgtacattattaaaagactaatccaagtattagggataaaatgggaatatcatacttcctggcctccaccttcatctgggagagtagaaagaatgaatcaaactttaaagagtcatctaaccaaattaattttagaaactcgcctaccatggacaaaatctcttcccattgctttactaagaatccaaacttctcctcggagggatcttggcttgtctccttatgaaatgctttatgggttaccttatctaaacactactactgaccttcctatgatcgaaacaaaagatcagttccttagaaattatgtatttggtctgtcttccactctttcgtccctcaggactcaaggtctggtagcacagactccaccccttgaattctgggcccaccaacatcaacccggagatcacacgtccttatcaaaggctgaaaagagggaaagcttgagcctacctgggaaggaccctatctggtgctcacaacaactgaaacagtagttcggaccactgagaaagggtggacccattatactcgagtcaagtaggcgtcaccttctccagagacatggaccattattccgtcaactcccaccagagtaacgataaaaagaaaaacttaatctatctctttttcttcttttctttcccttaactgcccccatctcatcattaatgtaactagatcaagtttgcccaaaattattacctttgatgcttctctagtcatgccttgtggaaatttacaggaccaaagacagcttactgcctcagataaatatctttgtccctcttgaacttcctcagattggaaagatataacaaattcttgtaaagatcatgataaccccaaaaatagtagctgtatgaactggtgggaatgggactcttgtccccttaaaacagagtccctatgctatacttggtctaatgtcctatggaacaccaaaggtccaggctggactgctcccaccagtctttgccagtccttaaagccatatatccattttacaaaagggattgcctcttctaattgtccatataaccagtgtaatcctattcggcttactattactattgcaacatcccaaaactctttcccttcattaagtcatatttatagcataggggccaacatcttgggaaaggatcctaggggaatttttgaaatctgctttattgccccttccacctctctaacacatcttcctcaaaccatcaccccacctatacccagtgacaaaaccaaagtagctatagtagaagtaaaagacctgaggtaaactctagccactgaaacagggtctcaggatgcaaatgcctggctggaatggattaaatattccatccacactcaaaataagagcgattgttatgcttgggcacagggcagaccagaggcccaaactgtcccctttccactcagatggtcttgtaatagaccagatatggactgcatggtagctctgctccaagacctcacagcctggggtaataaatcgtgtcaaagactttctttgctgttccctgagatcagcccccaaaggccattcagcctccatcttctgggaccaaatttgctttgtgtctctcacaacagggagaaaacttggctttccttggaaacctgacaggatgcagtgaggtcaagcacttccaagagctaacccatcagtcctcccttatccatccccgagcagatgtatggtggtattgtggtggacctttgtgggagactctgccaaataactggagtggtacttgcactctaattcaattggctatctctttcaccctgacatttcattcacaaaaatattaacaactaggcatcgtagtacaagaactgttcctgtgagtcttttgatcccaacgtttatatagatgctattggagtaccagatgaatttaaagcttgaaaccaaatagctgcagaatttaagtcattattcaggtgggtgactatcaataaaaatatagattggatagccaggcgcagtggctcacgtctgtaatcgcagcactttgggaggccaaggcgggcagatcacgaggtcaggagtttgagaccagcctgaccaacatagtgaaaccctgtttctactaaaaatacaaaaattagctgagtgtggtggcacgcacctgtaatcccagctactcaggagagtcacttcaacctgggagttggaggttgcagtgagccgagatcacaccactgcactccagcctgggcgacagactgagactccatctcgaaaaaaaaaatgtaggttgggtaaattatatttactacaatcagcaatgattcattaattgcacaagggatgctatgagggaatagctgagcaactaggacccaccagtcagatgacttgggaaaatagaacagcactagacatgatactagcagaaaaaggcaaggtttgtgttatgattggaactcaatgttgtacttttatccttaataatactgcccctgatcgaactataataaaagcactacaaggtcttactgcttcatcaaatgaactagctaaaaactctggaataaatgatcctttcactagcttaatggaaaggtggtttgctaagtggaaaggactcatgtcctcaattctcacctcactggctctcataatcagtgtacttatccttgtagaatgttgtattatatcttgcattcatggattagtgcaaaggcttatagaaaaggctcttaccaaaacccctttcaattctcctcacccttactcagataagttgttctttctaactgattaagaagagcaacaaaatcaagatatgttaaggagatttgaagaggaagaactgtaaaatcaagagggggaaactgtaaagaataaaagtacctctccaaagttttcctttttggttaagaatgataagtgttaagaagatagttcctcttaaaatctttctttaagtttcccttactcttcatactaacaaatcttctaatttactaatgattctttattgtgctccaagtagttgttacatacagtaaagaaataaacacattctatgttcttgtacttgaaccaaggcctaactcctagtctgcctggatctgaccagacatgcccagacatgtcccagcttgcagcctatgctcttgccttatttggaaatcttattgtcttcctggtttcccataagtgaccccctcctttcctttgttccccattgcacctttaccttatttaggaaagttaaagtttttagccaaccgggatcagtttagactgtgcggtccagctccagccaatggacacaggacacaatagtagaaacaacttgcgttaggaataaaaacccctgctttcccttgttctgggtgctctcgtggcaatcaggcttacaggtagcacccttctgcagaagtaaaaattgccttgctgagaaatactttgttcaagtgctcattttctttgtgacttcgagctttatttctaacagactcaaacaaaatcagaaaagaaaagagaaacattacaactgatattgcaaaagtacaaaagaccatcagagactattatgaacaattgtgtactgacaaactggaaaacctggaggaaatggataaattcctggaaacaaacaacctgccaagattacattaggaagaatagaaaacctgaagccctaacatacatgaaccattctcacagtggtcccagtgggcccctgacctctctgtgattatttcctcagtgcttgtttgcataattgaaatagacctactcagcaacagccaaaatccacagatcagttcctttttttaacttttattttcagttcaggggtacaagtgcaggctgttttacaggtaaacttgtgttgtgggagtttggtacacagattatttaatcacccaggttttaagcctattacccattagttattttccctgatcctcttcttcctcccaccctccaccttctgaaaggccccagtgcccagtgtgtgctattcccctctatgtctctatgcattctcatcatttagctcccacatataagtgagaacatgcagtatttggttttctgtttctgcattagtttgctaaggataatggcttccagctccacccatgtccctgcaaaggacatgatcacattcctttttctggctgcatagtgttccatggtatacatgtatcacgttttctttatccagtctataattggtgggcatttaagttgattccatgtctttgctattgtgaatagtgctgcaatgaacatatgtgtgcctatgtctttataatagaataatttatattcctttgggtagaatggtatttctctctttaggtcttcaagaaatcaccacactgtcttccacaatggctgacctaatttgcacttccaccaagagtatataagtattcctttttctccacaactttgccagcatctgttattttttgactttttaataatagccattctgactggtgtgaaatggcatctcactgtggtttagatttgcatttctctaatcatcagtgatgttgagcattttttcatgtttgttggctgcatgtatgccttcttttgagaagtgtctgtttatatgctttgcccactttttaatggggttgttttgttttttcttgtaaatttgtttaaattccttatagatattagacttttgtcagatgcatagtttgcaaaaaattttcccccattctgtaggttgtttactctgtcgatagtttcttttgctgtgcagagctctttagtcgaattagatcccatttgacaatttttgcttgtgttgcacttgcttttggcatctttgtcatgaaatcgttgcccatacctatgtcctaaatggtattgcctaggttctgttacggtttttacagttttggatcttacatttaagtctttaatccattttgaggtgattttggtatatggtgtaagaaaggggttccattttaatcttctgcatatggctagccagttatcccagcacaatttatgaatagggaatcctttccccattgcttgtttttgtcaggtttgtcaatgatcatagtttgtgtggtcttatttctgggttctctattctgttccattggtctatatgtctatttttgtaccagtaccatgctgtttcggttactgtagccttgaagtatagttgaaatcaggtagcatacactggacgtggtggctcatgcctgtaatcccagcactttgggaggcagaggcaggtggatcatgaggtcaggagttcgagaccagtctggtcaacaaggtgaaaccttgtctctactaaagatacaaaaaattagctgagcgtggtggcatgtgcctgtaatcccagctactcaggaggctgagaaaaggagaatcacttgaacccaggaggcagaggttgcagtgagccaggattacacaattgcaccccagcctggatgacagggtgagactccatctcaaaaaaaaaaaaaaaaaatcaggtagtgtaatgcctccagctttgttccttttgtttaggattgccttggctattcaggctcttttttggttccaggtgaattttaaaatagttttttctagttctgtgaagaatgtcagtggtagtggccgggcacggtggctcacgcctgtaatcccagcactttgggaggctgaggcagtggatcatgaggtcaggagatggagaccatcctggctaacggggtgaaaccccatctttactaaaagtacaaaaacttagcccagcatggtggcgggtacctatagtcccagctactcgggaggctgaggtaggagaataaaaaaaacctcctgaattcgttgatcttttgagtggtttttcatgtctctgtctccttcaggagacaatacctttgttaattttttattattacgtcttgtcttctgctagcttagggatatgtttgctctttgttctcaagttcttttggttgtgattttaggttgttaacttgaaatctttctaactttttgatgtgggcatttaatgctataaatttctctcttaacactgccttagctctttcccagagattctggtacattgtatctttgttctaattatcttcaaagaacttcttgatttgtgccttaatttcattatttacccaaaagtcgttcaagaacaggttattcaatttctatgtaattgtatggtttgagtgaatttcttagtctttattttgaatttgattgcactgtggtccaagtgactatttgttatgatttcagttcttttgcatttgctgaggagtgttttacttccaattgtgtgattgattttagagtaagtgccatgtgacaatgcaaaaaatgtatattccattgctttggggtgaagacttctgtagatatttatcaggtccatttgatgcagggctgagttcaggtcctgaatatcttcgttaattttctgtcttgataatctgtctaatattgtcagtggagtattaaaatctcttactattattgtgtgagattctaagtctctttgaaggtctctaggaacctactttatgaatgtgggtgttcctgtataggatgcatatatatttcagatagttagatctccttgttgaattgaaccctttaccattatgtaatgcccttctttgtcttttttttatcttttttggtttaacacctgttttgtcagaaacaagagttgcaaccctggctttttttctgctttccatttgcttggtaaattttcttccatctttttgttttgagcctatgtgtgtcattgcatgtgaaatgggtgtattgaagacagcatactgataggtcttggttctttatccagcttgccactctatgtcttttaaatggggcatttagcccatttacatttaaggttagtattgatatgtatggatttgatcctatcatcatgatgttagctacttactttgcagactttcttatgtggttgctttattgtgtcactagtctgtgtacttcattgtgtttttgtagtggctggtaactgtctttccatgtttagtgcttccttcaggagttcttgtaatgcaggtctggtagtaatgaattcccttggcatttgtttgcctgaaaagaatctgatttctcctttgcttatgaagcttagtttgtttggatatgaaattctgggttggaaactcttttctttgagaatgttgaatactggacctcagtctcttctggcttgtaggatttccactgagacatccactgttagtctgatgggcttccctttgtaggtgatctggcctttctctctggatacccttgacattttttctttcattttaaccttggagcatctgatgattatttgtcttggggatgatcttctcatggagcagcttactggggtcctctgcatttctgaatttgaatgttggcctgtctagctaggttggggaagttctcatggatcatatcctgaaatgttttccaaattggttccattctccccatctctttcaggtacaccaatcattcatacatttgtttctttacataatcccatgtttttcagatgttttgttcattccttttcattcttttttctctagtcttgtcttcttgtcttatttcagaaagccagtcttcaagcactgagattcttttctccacttgctctattctgctattaatacttgtgagtgcaccacgaaattcttataaagtgttttttacctctatcaggttggttatcagattcttttgctgattccttctcatctctatgggcttatctaccctcaatctttgaggttgctgacctttgagtggattttttttctcttcttttgttttctcttttaacagtctggccacttttctatagggctgtacagtttgctaggggcctgctacagtccctagttgcctcagattttccagtacctgaaggtatcaccagtgaaggctatgtaacagcaaagatggcagtctgccccttcctctgggaggtctgttccaggaaagtacagacttgttgccagcctgaacacacctgtaggaggtgctggagaccctggttgggaggtcttgcccagtcaggaggaacaggatcaggatctagaggtttactaaaggaggaatgctcccacaggagacacagaaatgaaccattgagctggaagctaagacagctacccaggcccttcagagcctcatgattctgaatcaacaaaggaaggagttactgcactgtctggggtgattggtcctggccatcagggggaattggtctactactacacaatggcagtaaataggactgtgtctggaacacaggagatcttttagagcatctcttagtaatactctgccgtataattaaagtcaataaaaacggcaacaacccaagtcaggtaggactagtaatgacacatacccttcaggaatgaaggtttgagtcacccaagcaggaaaagaaacctgatcagctgacggacttgctgaaagaaatgggaatatgggatggttagtgaaaaacaacaaaaaaagtatttataaatactagttgtggccggcacagtggcttgtgcctgtaatcccagcactttgggaggcggaggcaggtggattgcttaagtctaggaatgtgagaccaacctgagcaaaatgctgaaactccatctctactgaaaataaatacaaaaaattagtcagacttggtggtgtgcttgtagtcccagctacttaggaggttgaggtaggaggatcacttgtgcccaggaggtggaggttgtagtgagccaaggtaatgccactgcactccagcctgggcaacagaacaagaccctgtctcaaataaataagtaantaantaagtaaataaataaataaataaccagctgtaaccatgtgagcacttacagaaataaagacagtaaatgttatcagcattacttctttatgtttttataatgatatttatgtctgtgtgtatgtatccttacatgtattgagcaaatacctttcttttcatcttttcctgatgatctgtaataaaacataagatgtgctaacaataattcccttttattaataaaagttaacttttattacgcatggtatttaggttacagtatatcaaagagaagagtgcatatcatgcatctttttctgtggaaagggttagtgtgtttctagttggatgcaagttaattgtatcatctttggcaaaagtaagactttataattgtttttatttggagacgaagtaaggtttaaggaaaatgttaatgggtgccaagttcacaagcatggactgggatggtgggtcttatgtgtcaattttactaaaccatagcacctagttatttaatcaaacactaaatttatatgttgatatgatggtgttttgcagttgtgattaaaatctgatatcagttgatttcatgaaaaggaaattaccctaaaacatgtgggtgggactttttgaatcacttgaaggcctttgggcgaaatgtgagtcttcccagagaagaaaaaattctgcctcaagatgacagtagcaactacactttaggtttccaatctgccagcctggtctacaaattttagattgaaggctgcaacattatgactttccagccttccagcctgccgaggtgtaacttgtgacatcaataacgtaaaatgaggggtgggaaaagatgtaaagaagcagagtttcatattgatttcagttcaagattgttataactgtattgttatatataataccacagtaatcacaaagaaatatacataaaatatacacaaaagaaaatgagaaggcagttaaaacatatcactagaaaaaaatcgactaaatgaaaaatgactaaaaacaaaagaaggttggaatgaaggagatgaaggacaaaacagctataagacatgcataaaaccaacatcacaattacagaagtaaatccttccttatcagtaattactttaaatgtaaatgtaaatggattaaacttcccaatcagaattcagagattatcacaatggacagaataaaaaggatctggctgggcacggtggctcacacttttaatctcagcactttgggagacggtggctggcagatcacaaggtcaggagttcgagaccagcctggccaacatgatgaaaccccgtctctactaaaaatagaaaaaaattagccgggtgtagtggcaggtgcctgtaatctcagttacaagggaggctaaggcaggagaattgctggaaccggggaggcagaggtttcagtgagctaagatcgtaccactgcactccagcctgggcaacagagtgagactccgtctcaaaaaaaaaaaaaaaagattctcactttggatctaacgacacacagaagttgaaattggaaagacagagaaggatattcacaaaaatagtaactaagaatttttaatgatctcttatagggttctatgtgttggctgggctcagatgatggcctgggagtgttacatggtccagggattgaagtggaaggaaccagacgcaggcaggatggttaacagcagtgcccagactgacacagccacacttctgccatattttattgggtaacgcagatatcagatctgcttatgtttaaggagtggagagaacttacctcttgatgagggagtggtgggtcaaactaaagaaaagcaagtgcgatggaagatatttagatatttttgcagccctctttggaaaatacatgcaattcaaaacatcctatctgccagaaaggaagaacaccattacgccttacccctccagtttaagccctgtgattctatagtatcctggggaaggatcatgtcttaggaagcaggtggggagtcgaagctctaaataaagccccatctacacccacaatttcactttattttgatgaaattttgtgtcatttttgatgaaatgggggctcacaaatctctcttcagggatctctccagtggcggggacgatatgctcccttctccacagttctactttatagataagtggaactagatctcagtttcagttatttaaaacaccagagacttggatgcatgcatgtcacaaaaaggtgaaagaatgctctctgtcactttcccttctctcactcactgcgccctgaacaagaacctgctctaccctgaccttttgaatgaggaggagggtcgcactcctgcctaaccttcaactttccagagataaacctcctgcccaaacagcatggttcattataaagaccaaaccctacaccaagagctctccccttatccaccatggcagaaactgaatcctatggaaaggcccagaaaattctggctcgaaatgaccagcgtcatgtcccttccaccaaaataaagccaacatattcatcacaacatggggtattgataaggatatcaaaatgattaccaaaaaaatttgacaaatatgttctttttattacatcaacttttgtttccctccaaatataaagaaaataagcctgcacagcaggagaagttgttcccctgggagagcatcctctttccagctgaaaatctggtactggaatcactagaatggaacaagggtttctggtatgacacagcaccaggtcttggggcagccagagttagggagggctcaccctctgggtgacaccaacctttggttctccatttttttgttctcattgcaagaacaaaatttcagagtatcagagccaaagaaatttactctattttattctttagaaaatgcaatctcaaggataatcagactgttatgggctaaattgtatccccccaaaattggcatgttgatgtcctaacccccagtacctgagagagtgactgcatttggagaggttttaaggaggtcattaaggtaaaatgaggtcatgtgagtgtgccctaattcaatgtgactagtgtcttcataaaaagagaagattagacacagacagatacggaggaatgaccatgtgaagacagagagagaacacatctacaagccaaggaaagaaccttagaagaaatcagcactgccaacaccatgatcttgaacttccagcctccagaactgtgagacaatgaatttctgttgtttaagtcacccagtgtgtggtactttgttatggcagctctagcaaattaaaacaaacatattatattgcccaatggagagttttacctgactccactctggctccaatttgattaaggtctatatgctctgggacctctccagctcctcatgagaaatgacaaagaatgccatgtccagctcctggggtccctgatggcagtgagaggcagctcctaatcgagggagctttggggtgacgaaggcctcatagacttcatcactgttgggccacttggcttcagtcccctgaccacaatggatccatgggcctggattcacctctgacattcgtgtcttttttttccacaagtggtctctggtagagatggagggaaaggacacagggattcaaaatcctgactatcctagtgtcctcccctcacttccactcagagacaacacggccatttctctaaactcctgaggacgtggccaacataagtaatggagcacggggaggtgccttgaacagagctgcatggaagggtggctggtggcctctagaccctttgtctatacgtgctgggcttctttagtatgaaatgtcccagagacagccacattctatgcttggatcctcaaaccaaaatgtgtgagggtcttagccctggccaactgagcactgaattcaagagagaccaggaaaccttctgaatattgggacactttattcctgaatcctgaatcctggaagttggcttctggccagggaggcaggtgtcattatatctgggctgcctctgtgaggcctcttttccctctgcctttcacatctttctacccctgttaaaatcccagaacagatagaaacattctgccttcttagatgcctcttgctacaaatttcaatgccatttgattgaattaataaatgaaagcaactttaatacaagatgaggtttaaagaatggtatttgacatgggtttagaatgaataaatggatcaagtaatgaattttgagaaaattcatgtaaaatttatttttaaccattttattaacttgactgtggaattaataaatgattccaaattaatacaaaaaagtgcaatcacaaatgtatgaatgcccagaagtgtgaatgaggggcaatgcacacaagaaaaaaggtgtgcacaggaagaaattcttcattccacgtttttactgatccctctggtcaagatgggctaaaagtccaaaaatctgctttcctcacatggtcaaatgattcagcataaaatacaactgactgaacacaattatcaatgctttaaatatattagaaaaaatcaataaggaaaaataatcctaccatcactaagattttaagttataataaaataaagcctagaaacgtatcgtttgttcccaattccaatgattataggattgcagtgtttggctttcaaaagaccatttaaaacaactcagggaagttttcttaacaaactctatagcaggaccacagctaaacctatgagtagtagtagaagtaaacaatgacactgtctttaaaatttcattttgaaaaacatgtttgaaagtggctgactggctcctaatttaaccactaccatttgaagtcatatgttagattggcaaagtcgtcgtaatttttatgtattttatttttgtaatgcaagtgcttcactctgctatctgataaaagaaaataccaagccaggcatggtgatgcatgccttgtagtcccagctactcaagagcctgaggcaggaggttccttcagcccaggagttcaaggctgcagcaagctatgatggtgccactgcactccagcctgggcaacagataatgcaattttaatgtaaaattttagtgttaggagttcaaccttgaactgattctgtgagatcctatttgagagaagctgttgtgtacaatgaagtttggggaatatcaacaagaagtaaatgcctaccttgtttctaatttacaaactaaaaggaaattccataaacttttaatgctaagctaaatcattgtagaatgaatgacaaaaaaattatcctacaaaacataactagttgctagatatatttgaacattaattagaaaaaagatgaatgaaagccatgttttaggaataaaaactacaaaagaactgtaaaagcttatcatgcccttggctggagttacgcaaagttactactcacccgtaaattgggattagcatggtggctacttcttaaagttgatacaaggattaaatgtgttttaatgcatcaaaagcatctagaaaagtgcctgagacatagtaagtgctaaagtctctctaagtaaataaaaaatatataaaagtgatgctaaattcacgattctgatcctctgtgcacacccaatgtgtttaaaattctgttcatcttcccttccacattcacagtgaaatgtttctgattaatttaatgggctgtgtgaggtgttcattatcttttacattaaacttggtacattgtacaacactcttatattgtgataatttaccaagctaacacaggcttaccttgtttttaatccttttcacacacagcatagaattatattgctatatttttaaatttcagatgatttgaaaaatatttacactatagtagccaaacagtaataattataagcaacttggataaaacatttgaaattagagaactatatttagaataaatttctcaccttacagaaaaatttcccattgaaaacccctttatttcgcatacaaataagaattcagcatgtttttctatacccagagagaacagagataaacagggtcctttcgtgtggtttgggtattttattagatgctgagttcaagaaagtctcacccctgaagaagctgaaattccatttttcgaagcttgctcctcaattccacatgcttatcatagatatcaagcagtgcccttgctgtctgcccatcttttctgaccctgggacaccaccctttatttactttccccacaactccttgaaggtcaagcccccttggctgatactctgggttatcacagtaaatgtgccctctatcttttgcaggtcactggaaaggagggttcctaaagcattcactcctgaccctgagggaggtgggtgcactcactccaggactcaggttcaccacagataagcaaaaaagtcctcacattcaaaattgtcctgggccacatgcagcccacgagccgcgggttggacaagcctgcatctagaacgtatgcaccataaccccagacatttttctgtttttactttaaggatgctttctaaatgcaaaagcagtcatatccctagcagacaacaaatgtcagttgaatgaatgatcactgtagagcacctccctattctaaagcccatatctttattaatgtagccacagggcaaatgctgttttgtggcagctaaagaacataagttcacgtggacatccatgccgccagtgcttttctcaccagggctgcttgtgtgtcctccctcctcccacaccaaccctcctgcacacttcagcacaaaactatatatttctcttcaggaacaataaccctagccttatgggtacaattttccaaccacatatgaatctaaattagactctgctttataaatctatgagtttggattggagccagcactaggattactacaactcagagcaggaagaagagtaggagggcagaagaggagttccaacagaaagttaaccatgatgagaaactatgggacccctcctctctgcaaatgtcacaatctggttcctttaaaaagattggagaaaaatcgaaaataccattaaggaaaaagccctgggaggaaggcaagacctggacaggtctgaaaatttgtctcttgataccacaaggagatttgtatgcagggaccaccctaggtgacatccaactccctgtgatcacaggtcttggtgggacaaggttctactgaagggccaaggacaatggagcagcaaagatgacccaggtgagctgtgaccacataaagcccatggtggccagaacagaaactgggcacagccccatctactctcctcccctgcaacaaatcagcacaagaaacacgtggactctggaaggttctcatgtcttccatttattttgtctctcaaattttaggaatcttctcctttaattaactcatcaacctctcatggcaagaatttgagaaagtaaatttatactcaggttctaattttaatagggaaggaagaagttacagctcagtgcaccatgaagttgagacagagatggagacacctcagccccacctctctggaacaggaaagatgattggggagggagcacaggtcagcgtgggaagagggtcatggtggacatgggggtggggtggtctccccacctcctcacattatgcctacacgaacacagacacatgcaggtgcctttgcagaaacaaagtcagggttcttcaagtcacaaagggaagggcaggaacaactcttgcctctcagtcccacacaaggcagctgtctcacactatagaaaaaaatattcatgaacaaattcatatccatcacagtgaggggtcacaccttaaacagcccatcgcatgctcaatacatccaatgcaaagaaaccccatagcacagctgtgtccactgttccgcccaacacccaacacacattaggtcctccaagctctcacctttacaagctgtgagagacacatcagagccctgggcactgtcactgcctggggtagaacaaaaacagaacctggtcagatcccacagaagatgtggctagaggaggaattgtggggtgggtgagctcccccatgggctcccaaacacaatatcccaaggacctcaggcatcagcctccttcatacttacttgcagcctgagtgtaactccctccttttctatctgtgagaagaaaatgtcctgtgagataccagaaaggagccagggccttaaggtcctagaggaacctcctagtcttggaccccagagaagtttccagaaatgtgtgactgcagacccagggcgggatcaggaaacatgaagaaagcaggtgtgggtcctggaccaatagccctcctgaggtctgtcctcagggaccttcccctgtgacttgtgactgctgggatcaggtcccatcaccgccgtaatcaaggtgataaatctgtccttcattttaacaggtgctttacaaaagagtaagtgctggcacacagggcccaggctgggtaggcccatgattgtggatggtgcttcccagtaatgagacagggcacatttctagctggggcttggaaccctcagtgagacaagaaatctcagaccccacccttcaccccttctccacctgagctcttcctcctccacatcacggcagcgaccacagctccagtgatcacagctccaaggagaaccaggccagcaatgatgcccacgatggggatggtgggctgggaagacagctctgggaaaagaggggaaggtgaggggccctgaccctgctaaaggtctccagagaggctcctgctttccctaagagacatgacacccccatctccctccttaccccatctcagggtgaggggcttgggcagaccctcatgctgcacatggcaggtgtatctctgctcctctccagaaggcaccaccacagccgcccacttctggaaggttccatcccctgcaggcctggtctccacgagctccgtgtcctgggtctggtcctccccatcccgctgccaggtcagtgtgatctccgcagggtagaagcccagggcccagcacctcagggtggcctcatggtcagagatggggtggtgggtcatatgtgtcttgggggggtctgacgggaagagtcagaaaattcaggcattttgcatctgtcatgggacactccaccagcacgcatgtggccatcttgagaatggacaggacacccgggatggggaagagagcacagaacccagacaccagcctggacacaggcacctgggataatcttctattccctgagaagggaacagcgacttctggtcctgacctgagtggaggctgagggactcagaagtgctggactcagacccccacacacattgagtgtgaagcagagaacaaggcctgagaggaaaagtcacgggcccaaggctgctgccggtgtcaaagggaaccactcatcagtattcgagggatcgtcttcccgtcactccttcagagattttatcccttaattgtgtcagagagcagggcggaacctcagagtcactctctggtacaggatctggaaacccaggaggattcctctccctcaggaccagagggagggtgatattctagtgttggtcccaattgtctcccctccttgtgggaggccagcccgggagatctacaggcgatcaggaaggcgccccgtggcccctggtacccgtgcgctgcagcgtctccttcccgttctccaggtatctgcggagccactccacgcacgtgccctccaggtaggctctcaactgctccgcccaacgggccgcctcccacttgcgctgggtgatctgagccgccatgtccgccgcggtccaagagcgcaggtcctcgttcagggcgatgtaatccttgccgtcgtaggcgtgctgttcatacccgcggaggaagcgcccgtccgaccccacgtcgcagccatacattatctggatggtgtgagaacctggccccgaccccgcggtcagcccagtcccccgagccccgcccagccccgaccaacccgggggaatttttggcctaaactgaaaatgaaaccgggtaaaggcgcctgggcctctcccgggtcaagggtctcggggtcccgcggcttcggggtggatctcggacccggagactgtgggcgacctggcccgtccgtgggggatgaggggtcgtgacctgcgcccccggccggggtcactcaccggcctcgctctggttgtagtagccgcgcagggtccccaggtccactcggtcagtctgtgactgggccttcacattccgtgtctcctggtcccaatactcaggcctctcctgctctatccacggcgcccgcggctccatcctctggctcgcggcgtcgctgtcgaaccgcacgaactgcgtgtcgtccacgtagcccactgcgatgaagcggggctctccactgccgggccgggacacggatgtggagaaatacctcatggagtgggagcctgggggcgagcagtggctgagacctgaccgaccctcctcccggcgcggctcccccggtcctgcgcccccgccaggagggccccttgcttctccccgcagaggcggtttccctcccgaccccgcactcacccgcccaggtctgggtcagggccagggcccccgagagtagcaggaggagggttcggggcgccatgacggccatcctcggcgtctggggagaatctgagtcccggtgggtgcgtgcgggctttagaacagcgaccgcgacgacactgattggcttctctggaaacccgacacccaatgggagtgagaactgggtccgcgtcgtgagtatccaggatgaaggaccctacgtaggttgggagagggagaaaagaaactgcggagttggggaatccccaaggctgtgactccccaatccatacaccgccttcggggcctgagaccctgagagccacgcctggggccctgggacttcgccctgacccctctgctcctgtgcgaagcgctctgtctcaatgtctccctgagtcttggcccaagagctgtctgagaaacaagggagaaaccctcggttcacttttcatcccattatctctgtccctgaactggactccctgactcccactccttacctgtccacctggactcttctagaagaaaaatcaccccatggagtttggtgccagagagtaagcttgctctgggaatggaggtgtagatttttgttgttgttgtttttgtttttaatccggaaaagttgtgcctgagtgcatgagacagaatagagaccaatttgctctttgtttattaactacagtgggtagcagaatcttggtaacccctgaatgatcaggaatctaataggtaaaaaaatgtgactttggccccttaatgtataaatgtgtccaaaagcattacaacaggactcacaaagctactaagttgcactttcccagacaatgtatctgtgactcctgcttgtcttactttaaatttgccatcattccttagctctgagtttctgtgtgagtccacgacatctcctcaatacaaagtagccactgtgttcctatatgttgcaaccagaagccattacaggctttattcacctcaaagttgcaactgttcaaggcagtcacaatgcccctcactagtgctcatgcactgcctgtttttaggaagtatccacttctaagtgttgtgtatattttataggaacacttagtaatttttaaacctgattaacataaaaaatagtttttaggcagacccacatgaggtattaaaagacaactgccaaggacaccctgctaggctctgtagattgatgtattaaaatccataaaacaatgtctttaaacctaagaattctgctgctttcaaattctttcctctgctccttttcctcacctcctgcttctccagcccttccctccatccttctcatccttcaggccctcctctccccttagtccccaccacactgtcactcatgaattgtgacactagcactgtcccatgacctgctacgtaactgttcttttcacagtggccctgctcctgtgagtcggagtgtgtcatttctccacttaaaacactccagtgtctccacctcggtcttgtgaagcttctggagtgtcaggcacttgagcatatgaggggatacctcgttcattgtagggactaagttaatttttgttgacttaatgaatgaaatatgagtgtattaaattgcatcacagataattataaactgtaaaacactgaaaaagttcagaaagattttattttatgcaactagtgtgtatatcaattcatcaattcattcgcacactaccatgcctggcaaaacaacacacatttatctgcttatagttccttggtcagaaatctgggcaagatgtggatagaatctccattctgggcttccaaaagctgtgttttcattttgaatcctccttcgggtttatacagaggtggcagaatgcagtttcttgcagttgtaagactgaggtccctgttcctggctagctgtcaatgtagagaactgggaggggctcaattcccggtgctcaccagcgttctttcctacacagccccttaatttttctttttttttttttttttgagacggagtctggctctgtagcccaggctggggtacagtggcccaatgatctctgctcactacaagttctgcctcctgggttcacaccattctcctgtctcagtcccaagtagctgggactacaggcgcccaccactatgctctaattttttttttcttagtagagacagggtttcaccttgttagccaggattgtctcgacctcctgacctcatgatccgcccgcctcggcctcccaaagtgtcgggattacaggcgtgagccaccgcgcccggcccatagccccttccttttcaaagcccatggtggaggaaacccctcatgctgagtccctttcacactgtaagtctctatgctcatgaagaacccagtcctttcaagggctcacctgattaggacagtccaagcaggataaacccagcataaagtcaactaattgagacccttaattatatctgctaaatcccttcacagcagcacctacattagagttggttgaataactggaggaaggtgaatgaccaggagctgcttgttgaggccatcatagaatcagcctagcaagggttggatcttccttttgtgtttaattgggacacagttggaaagtgaagttcaagtaacgtgatcattgtgaatggtcataaaatacatcctcttcagccatggaaattgtccttactttttaaaactaagttacatgtttaatatcttataattaatttaggccaggtgtggtggctcacatctgtcatcctagcactgtggaaggcagaggaatggagatttgttgactccagaagttcaagatcagcctgggcaaaacctccatctatacaaaaaattagaaaattagccaggcatggtagttcatgatggtagtcgcagctactcaggaggctgaggtcagagagtcccttgaccccaggagttcgacactgcagtgcatggtgatcatgccactgcattccagcctgggtgacagagcaagaccctgtctcaaaaataataatgatgatgatgacacatttagagcaaatgcaatttgatgtgtaataatacatcctctcttgtgaaaatatattatttactgttgcataacaaattacgtaaaacttagctcgaaacaacaaatgtttctcatctcccacagtttccaatcgtcaggaatccaggagaggtttccctgagtgcttcctgctcagggcctctcacaaggttgcactccagttgtcagccaggggccgcagcatctgagggcttcgctggggctgggaattcacaagaaacatggatcagtcacatggctgttggaaaaggcctggttccttgttgtctgttcccagaaggcctcagttctcagtcatgtggaccttcctgcagggctgcttatggcacagcggctggcttccccaagagctcatgatcccagattcagagagagagaaggtggaagccgcagttagttttatgttctacacccagagtcacaaactgttatgttggcactattctataagttagaagttgtattagtccattctcacactgctataaagaaatacctgagactgggtaatttataaaggaaagaggtttaattgacccacagttctgcatgactgaggaggctgccccaggaaacttacagtcatggcagaagtggaagcaaacccgtccttcttcatgtggtgacaggagagagaaatgcacaatgaagcagggaaaagccccttataaaaccatcagatctcatgagtattccctcagcatcaggagaacagcatgggggcaccgtccccatgatccaatcaactcccatgaggtccctcccccaataggtagggattacaatttgcataacaactcaagatgacatttgggtgggaacacagaaccaggccatttcagaagtgcctcattaagtccaagccacactcaagagagggaattaagctgcacctccagaaaggaacagtatcaaaggatttgaatatatgttaaaagcaaaattataactattgtttcaggtttctgaaaatcaaagtcttcttgtatctaattatttttcattaactctttaagcttgtcttttaatttaattttttttttaagttccagggtacatgtgtaggatgtgcaggtttgttacataggtaaatgtgtgccatgttggtttgctgcacctgtcaactcatcacctaggtattaaccctggcatgcattagctatttttgctaatgatcctctcatcaccaccctcccccaacaggccccagtatgtgttgttcctctccctgtgtccatctgttctcattgctcagttcccagttatatatgagaacatgtggtgtttggttttctgttcctgtgtgagtttgctgaggataatggcttccaccttcatccatatccctgcaaaggacttgatctccttcctttttatggctgcataatattccatggtgtatatgtaccatattttctttatccagaccatcattgatgggcatttgggttgattccatgtctttgctattgtgaatagtgctgccatgaatgtaaacatgcatatgtctttataatagaatgatttatattcctttgggtatataccctgcaatgggattgctgggtcaaatggtatttctggttctaaatctttgaggaatcgccacactgtcactgtcttccacaatggattaaccatttacttttccaccaacagtgtaaaagcattcctatttctccgcaaccttgccagcatctgttgtttcttgactatttaataattgccattctgactggcataaaatggtatctcatttgtcgttttgatttgcatttctctaatgatcagtgatgggagctttctttttcatatttttgttggccacatgtatgtcttctttagaagtgtctgttcatgttctttgtccactttttaatgaagttttttttcatgtaaatttgcttaagttctttgtggattctgaatactagacttttgtcagatgaacagatggcaaaatttttctcccattctgtagcatgtctgttcactctgattgatgatagtttcttttgctgtgcagaaaaaaattgcctaatctgattgcttttcatttacaaaaatgagaatctcatacttcagttaacacatctagcttagcatgatgccaataattttggcttgatgctgtagcccgaaatggttagctcgaattgagagacaagttattttcaaaaactggctatccgatgggcattctataacacacttaacatcttttgttgtggtattgaatgagatgtgatagaagtgatttggacaccactatgattctaaatagcactgctattatgtgcttctgaattttttttctttttagcagtcctattctgtttccattcatatttgctattccatcactgggtattccttcctactcttttcatgtttcatttattttgatcagtgatgtgcatttccaattctgtaaaagtttaattcagttgtatgtgtggtaaaatgtaacatagaatctttgcaagatgaaattaccttgcacagcatattgaacattgtattattgaaaatctagagagatgccagtgagccaaggatcgaatgacctatatgtagccaaggccgttttcatggcatcctggtcctctctgtcacatagctccttacaattctctgtggttttcttccaatgtaagtagttcttgttaagaatctttgcaatgaactttgaaattcttttctttttcattgctataaaaaacaggtatattttaatttggaaaactctgtttaaggaattataatcaagtaactaccagggggcccagcactggtgtaaaacacaaaactgtaaaactaccctgggctgctacccttcgaaatggggtcttttcaaagttagtaacaatgaagctgtcattcacacgatgcatggctgaacaaaaagacagagacagcgggtgctgcattttatttttgtaatgttgccttgtccacaagacactatttacacttaaatcaattaaaattatattaattttaaaagtcagttttcccagtttctcatttgtactagccacatttcaaatactcagaattcatgggtggccggtgactgacgtaatgaacagcacagatataggcattccatcactacagaagggagctggacagcagtgtgccaatgttttaaggacagaatcctgagtagaattataggatgatgacaattccgagttcccattccaattcatggtctcttctctgatgagtggtgtagggggaaatccatctcttttgtgagtaaaggttaatttttccaactacagataaataattaacattactttttttaaaaccagttttacactgttaagttacaaccaaaagtgaagggcttgaattacatttacattttaacacatggttttaaagggaagatccgtgtgaaaaagaaacaaaactcagaaaaaaaacccagaagttttcataccaattcccatagcaataacaatcccttttctgtcgttagagttcaccctcattctgatttcatagtaatcctgttcatgtctttctttaaagctttaccccatgcatatatttctaaaacaacacaggttatctcaaactttatataaattttgaatttttcatacaaatggaatcatgctgtgtatattcttacaagacctcattttagatcaaatgtatgtttgctaagtttttataggtgtctgtattcatgtttattatttgtaacatttcattgaataaatacatcatatattaaattaacccttctctcgtttatggtcatttcatttgttgtcagattttttaaaattacaagcaatgctgttacaagtcttctagcactagtctgttggcacaatgggcacatatctgtcaaaattatacacctaggagtggagttactggtatatgtgtatgatatcccactcaaagagatgatgccacactgctagataaagtggctgtggcaagcaatgtgtggtagatcttgatggtctgtattctcatcagtgtttggaaaactcaataattttaatatagaaattctagtgcatttttcaaaatatctcattatgttttcctcattaaactttattgagatataaatcacatactactcacccatctaaagtatgcaattcaatggtttttcacatattcagagttttaaatctgcatatttaattttagaacattttcatcatcccaagataatcaacggctttataatatgtctctctatacaatgaagtaaatccataaattttgagttttcacttcaggagtgttttttctatcattggcaactttcctagcttgacaggcacctttccaagtacctttaacaattttatttttaatagagatttcattcagtaatcagatttggagagcactgacaaattttgccacattgagacttataattcatgaacatgtaatgtttacttattaaatactcttaaataaagtattgctggttttacttgtgcatcttttagtagatttattccaaagatcttgaaatatggtgctagtgttcacagtatctctttctttgttgaatttcagtgttggttgtggtatgtataaatataaataattttatatagttttaattcaatgactttgcttaaagcttttattaattcatgtagttgatatattctgttgcctgattgcactagctaggaaatccagtgtgattttgagtgcaatggagagggcaggtctgctggtatcttctctgtgcactcctgatccacactccatgtactctccctgctccgtgtcctggacactgtgctgcatggcctgctggaccacaaaaggcagccttgctttctggcttttatttgggatcggctaaatgggagcatcatcacggagagagtgatgaggagcactggttggagggatttctccccatagctttcagtattgacatagggagaaatgagatgtgattggcctggattcatcaatatgggatcttcacccagtatcttagattaaatgtgttattttgaacacctaagagtgatgttaatagtcagccaggttagctaattaaaacccgaattcaactgtggcctagcacagtgtttctcaaatttgactgagctttccatcacgtgggagattttaaatagccccaggtctagatcaacatgtaaaaccaatgaaatctaattcatcaaaagtaggatcctggaaacagtattgttcctaaaactctccaggtttgactccaatcaacagccaagttaaaaactagtagctcaaagcagtggttctcaatatttggtatgtatcaaaatcatctgggaattgtagcaaaactacacaggcccagatattattctaatttaacaggctagagccttgagtttattaaaacaaatcatctctccagattttgctatatgcaacagtttgtgaaaaccactagcctatatcaacagaatccctatcacaggcatactgcagaggaaagtgtctgaacacagctcagagagacggaaaagtgggaatgtatccattatgtgcaacctacttaacatctcctaaccacatctcccgggagggctgagaggatgccccccttaccaaggcattaaggtgccacatgacacatacaggtataaaaactgctaggtctgctgagcagtcaccaatcttgagtcctggatattcagaaattcttatccagtagctacaccaaagccagctgatatgtcagagcccctcaaataagcagaaggctggtgcccttgagaaaggaccctataattcactctgagataaattctttttacaggactttcccagaggaacatttagtcatttatcgctgtccactggggaatggaaaaccccaaataatctgggagctcaactttaatgtctttaatcctgtcaaagattaccctggggttaatactttgttgtaacctgcataaaaatctttccctacactaaagtaatgaagatattctactatattactgtaaagagatgagtacaatcacatttttttacaagagatgagtacaattgcatttttccagatagataaccaattgttgttaaattattttctaaatcgttttcctttacccactgatctgtaatgttgcctctgctctacatacgttcagggctttctattctgttcccaagttctagtttactaccctgtctcaacatctcgactctcttaattaaaataactctaacataagtcttaatatctgctaaggaaattctctctgcttattattcttatttagagtgtcatagctactttttctctttcatatttcaatataaggcttaaaattggcttactatgtgttgcaaaaatgcttgataaacatttgattgagatgtgtttgagttcttagagtactgtaggaaattaatgtcattattatattgaaccatccaagccaggatcctggtaaattgattcagttaattcactgtcttctttaattactgtcttctattattactcagtagggttttttaaatatcccaattgatatcttgtacagaatttgttaggtttagttttagaagtttatatgtacgcacttttgctgaatcaattttttaaaattttcattttatatctgtgcaaaacagctctataatttcttctacctaccaagcaaaaatatcttattaacttccggatcacgaggtcaggagatcaagaccatcctggctaacatggtgaaaccccatctctactaaaaatacaaaaaattagccgggcatggtggcgggcgcctgtaatcccagctactcgggaggctgaggcaggagaatggcctaagtaaatctgggaggcggagcttgcagtgagccgagatctcgccactgcactccagcctgggcgacagagcgagactccntctcaaaaaaaaaaaaaaaaaaaaaaaatatatatatatatatatataaatatatttatatatatatgtatcttattaacttcaagagtgttctaatcatttgtattttctgcattcagcatattaccatttgcaaattataagagttttgtgccttcttttcaaacctttttaagtatatttcttttcttctttatgacactgggaagatcctcaattacaatttttaataagagcagtgaaaactgctggaccactcaaacaaaaattgagctgccactcctgagaactgactgtgttgctcttatggatcagccctttgggacccatcctaatcccagggatttacagtgccacatatgcacccgcagcctttagatgtagactttatccacctagccctcaaaggtctcagacactgcccagagcagccaacatcacccaggacaaaagcaaccctgcagctcaacttgctcttctagatttctgccttctcctggttcctggcctggcaaatctttactgcttggttagccccttcatactttccatcagatttttcaaatatgtacccttttcccacttgtccagtagtactcaatgggagggatgatccaaattacataattaataattgctgaaaactaaagtacaatgtacaatttttatgtcaatgtcacatgccaagggagaaaatgcataggtatcagtagtgactacttaagcatgtgtaacttagaggtaataaaaacaaaaatgttaggtaatatctatctattccagattaaaatctacatcaataaattcaaacttctaggaaaaatatctgcatatgtaatatatgactgtgactccgatagacgaacgtgggaatatagtatgaagatacttctcatcgtcttcactctgtgaggcagtcagtgtgaaatgagaaccagggattcaaaatgtgttgcaagactatgaaaagcactagagtaagttctacacaaataaagcaatctcctcataaattacactgggcttatctgtgagatgcatgtccttatcaaaatggagttaattctaggtaggtgatcttgtttaaataaaatggactctgactcactagctactggtccaccatcggtttttccctttactaaaagggaggccccacactgagccagcatttcccaagaggaggtgtcttgctgacatcaacttccaacagggtaaattttcatgggaacccaaagagaagaggaagacctcaagaaaaagggcctaaagaaatgacaagaatgataacattttacacttttattgtgctatgtgtcaaatagtggtcttcgcactttaaaaatgccaattcattttattctgactacacatccatggcttgtgcctgttatttttccctttcatagttaatgccataggaatgaagatcacacaaaagtagggatggagccgggtttgcacccagacaattctgccccagggccatgctcacaccactgcacttttccagaatgtgagggtgggcggagagtccagctcagggagagagattggagtgagagaagaaggagagggggccgactggatcactgtgatggtctggacccacgattccaggagagaaagacaaggattatgtcactcagggaaaatatccaaagtctctggtttaaacttaagcatctccagctccaggacaggtggctggtgggaagacaaactaagccaaagccacagctctgaagatttccctctcctgagaattctgcaggggtttccctgacctcatggccacctctcacccttggctcttgtttttcccccaggactgataagggcgtccagatgggacgcctttatcagttggtctcgggttctagagaagccatcagagtcagtggaggatgagcttcataaagtgggagatctccaggatccttcctggactccaagatacccagagaagccggatcctgtgtcccggaacccccttcctgttcctttgtgggccctgaccttagggagaactgggctggtgagaagatcaggatgaactgggctggggaggcaagagggaaagggcggcttggagggcttagtagctcctctcgcggcgcctccgtcccgggctgggatctgcagactcctcaggtcacttctcccagagccgccgccccaagccaccctcccctggttcccgtccctctgtcccctccccagctccccccacacagtaagaagctcccaagtgagcggctccggtgccgggcgggacgtgggagggaggctgtgtccccgcggggaggcagggcgacggcgtccaggagagaggggagcggggcggtgtcccaggcaccctgcgcctctcccgcccgcaatgtgcgggttcccggagcccaggcccgcaccccgggtacttgaaggccgccgggctccgccttcaccacgtgggccctgagttcctccgcggcgccttcgtgggcgacacgcgattcctgagcttccacagccgcggcgagaatcagagcgtgaagcgcgggtggagaagatggcacagggctgtgggcgccctgaggaaagttctcgaagaccgcagggaatgtgttactgaggggcctgaggttcctgggcccgcaccaccaccagggtcagccggtgagtgacctcagactccgggcacaggtgtgccccgccccgaccccagagtgaccagcccggttctcccagggatgggggggcggggcctcagctgatccctctcaggtggggaagcgctggggccattttgctccaggtttctccttcaatctggatcatgactgacttgttctgtgaccaatgacagagggaggaaggagctggggtgggcggggcgacctccctcccctccctggccccgcctccggctcaccctggggcaggtccagggcaccgcacctgcagatgctgctgtctggctgggtggtggctccggacaggagcttcctcgaaggacactttaagtccgcctacgacaaggagatttcatgtccctgaacgaggacctgcagacctggacagcggctcaaagagcggcttggaggatccatcacacctgggagaggttctggaccgcggaggccacaagggaagccccgatgggtcactgcactaggtggctgctcagacacctggaggatggcaaggagatgatgctgccggcaggtactggggtcctccaggccccacgtcaggacctcttggagctggaattatgttccttggaggcatccatatttgaggaaagaggagaccgaacagtctccgtctcctcctcctcctccttctcctcctccgtcgccgcctcctcctcctcctccctactgcggggttggggattctggcccaggctcctgaggctctcccatgggtgggtggggagggaggacagagaagaaggagtgcagggaggtaaataggaaaggacagtagttacccttgtcaacagtgatgtacttgggtctagcttcaccgggaatctttttctttcctggccctctgtgcccaacaattacatataatctttaacccaaccagaagataaaacaccaggaaacggaatcggcagagacacagtagatgaaagtaccacgaaggactgctaggttctaatagaaagtagcagatgtgagtatcaaaagttataaacctttttgcaaatcagaaaagaaacagactttttttttttttttttttttttttttttttttttgagacggagtctctctctgtcacccaggctggagtgcagtggcgcgatctcagctcactgcaagctccgcctcccaggttcacgccattctcctgcctcagcctcccgagtagctgggactacaggcgcccgccactaagcccggctaattttttgtatttttagtagagacggggtttcactgtgttagccaggatggtctcgatctcctgacctcatgatccgcccgcctcggcctcccagagtgctgggattacaggcgtgagccaccgcgcctggccgaaaaagacatttaaagactgaaaagtggtatcaccaatgttagaaccaaaaaactggacctgagggtgcatcacccgagggggttaagttaaactctgtccctgttccttctagagccccgtggagattcctgctgtgcagcaaagcctgggaggggcccctggctgtggaaggcaggctgagacgtcataggtgagataggaggaaggggacctgctggactcccggggacctgctggactcccggaggcctggtacccagggaaggccagttcctagggctgcctctggacttcagaaccttgatgttaagtcatattttcccgctcctccatgagcttccaggagcagggccacatggggctcagaaagtcccacagtgcctaggaagtgggtctgtctcttgtgggacaaaaattctcaggtctttctctttcagattcttaaatttcttaaaagagattgaactggaattacccaggccccaaactgaatcaaggaattgggactgtgtaatgtgcgtgtgtctgtctgtgcacatgtggatgtataaacaggtatgggcatctatgtgtatgtgcgtgtttataagtgcgtatatgtatgtatctgtacatatttatgtatataagcaggtatgggtatgtatgtgtgtatgtgtgtgtgttactctgtgacttcctcatacttggcaccaccctggccttttcctggttgccccccacaccataggttgtaagggacagaacagcgctccacctgcagacatagaacgggggtcaggataagtgttggggagagctggtttcatcacacctggaataaggaaacaggacctaaatggatgggtcccactcttttacagaattgtaccttaaaaaaagtgtcagtcacgttcctaagggatttacatgaattaaatcatttaaacctcacaagagccctatgacctagagacagctatcagccccattttacagacaaggaaactgaggtcactaggaagtttagtaactgtcagtgggctgactcatgactctaaggagacagtctgactgctgagcccaagtaactgctctgtcatactcaccctcacacccacctactactggctccctgtgggtcacctcctcctgtgggctctgaccagcttccctttcttctccaggcacagagactggactgaagtctattgcatttgccacagtcccgactggggagtcatggggttgaagtgacccataggctgatggagtgtgcttgctggatgcgggggaaagggagatttatatctgccctggagggagtcaatggtgtttgcatcatggcatatgaggtgcactatctagaatgctgaagggtaaagaccagcaggggccatgcagagtctattcatcacggttttatttcacagcctgcatgggcctgagagtggtggggtgttcactcatctccatccaatccctactggttcctgacctctcttcttccaactggtaatacctgcactcaaggttgaaccatgaggaggagtctacccacaccccacacttcctcctctctgcctgccactccctagtggggcctcctggatggtgtgagaggagagtgacggagatagtaagagaacttgacattcacactggtaaagagaaagtgggatgtcaacctatgagaaagataagggctgccacagggaaggggatgagataagttctaactggccccaagggtcgcttaactcagtgaaagctgtgggaggtgttttcagctatccattgctatgtaagaaattgccacaaacttagcagcttacaacaacacacatgtattatcccacatttctatgggtcaggaatccagacacagctatgcagctttctcacatgattttcatcagagtgccatggctgggtcttatctggaggctgaactggggaaggatccacttccaagctcaagcaattgttggcaggattcattcctccatgcctgttagactgacagcctcagttccttactggctggtggccagaggctgacttcagttccttgccacatgggcctttctgcttcatcaaaacttgcaaacaaagaaggtagaagacagagtcatctagtaatacagaagtgcggctgtaatatgacctcattacggaagtgacatcatttttgtcactttctattggttagaagcaagtttaggccccatcggttctcagctggatgagatgccaagagacaggatcaccgggcatcatcttagggtctctatacacaggctggaccatgactgaacaagatcattacaaacccgggtcctggactgggccagatgatctgcagagctgagctgtgtttatgaaatcgaatccctggaaacaaaaatcaagagacgcccactggaaaggctggtcttgcattctgtgtgaaagctaagatggcgttctctccaaatgtttttctctattacttttcaaacctcctgcatctgtaaagaagcaacaccactaagcctatctggtttttttttcattcgagtgtcatactgttaggcaaatcttctgctttcactatttcttcaagcttgttttcatgcagaatcccacgaacttctggtaattacactttattgatttcttttccttcactcagtttattttttagtgattttatttctcttgtatggataaattagtccatttaattagggaatttgtgtcttctaaagaaatatttctactattttttggaccatatgggctaagatcctcttgccaagcctcagctcacttcccacatggacacgacaaaattactgggggtcctggaaagtcggggattggcatagttcttaagcaggatcagacatgtaagtcacaaagatatgaggcagatacctttgatttccttatgaccctgatgacagcatacctggcactcaatacctgtctgtagaggtgaactcaactaaactccctgatcactcaataccatgtgagcccaaggacctcttatgtcatgggccaaattcccttgttttatgccatttttccagatggggtgctgttcacatggggacgtttctccttttccacctcattttccatgtgtgaaacactgggtcttgattactcagatctctcagggtcctttctaccttttcctccatcctaacattctccctacagttcccgccagggtttccccgaacctcttcttctccatgtattcagtctagcatctctcagttcattcttcacacttcaccaaagtgatattctggaatgcagatctgatcaaggttcttcctgacttcagtgctgccagtgctgcacattgtcctcggataaaagccaaaggttctccccaaccccacaagccttagtcattgaatctgctcactgctccaagcttgctttttgcccctgtgctcctctttttctaatctctaccctgaactgctgacagttcccctagactccctgtgtgttctttcttctgagcctttgtttgtgatactgattccctaaaagatttctcctgccattcacctggattaactgtgttcctcatccagttctctgtttagacattgcatcctcttggaagcagttttcccgcactaattatagtagcctgcatttagctgtttttttagtccccttgtggaatctttgatgtttcactcattgagggctaggagaccctgacacatagtaactgcctaataaatgtttgctaaataaatgtggggttcaccatgagataccacgttatacatgcaagaatggcattaatttggctgggcgcggtggctcaagtctgtaatcccagcactttgggaggccaaagcaggcagatcacgagatcaggagatcgaaaccatcctggctaacacggtgaaaccctgtctctaccagaaatacaaaaaattatctgggcgtggtggcatgcacctgtaatcccagctactcgggacgctgaggcaggagaattgctggaacctgggaggtggaggttgcagtgaaccaagatcataccactgcactccagcctgggagacagagtgagattctgtctcaaaaaaaaaaatggcattaattaaaaagtcaaaaaacaatagatgttggcatggatggattacactgctggttgtgagaatgtaaattagtacaactactatggaaaacagtatggagattccttaaagaactaaaactagatctatcatttgatccagcaatcccactactgggtatctacccaaaggaaaataagtcattatatgaaaaagacacatgcacacgcatgtttatagcaacacaattcataattgcagggatgtggaaccagcctaagtgctcattgaccaaccagtggctaaagaaagtgtggtgtatgtacaccatggattactactcagccataaaaaggaatgaaataatgccttatgtagcaacttagatggagctggaggccattatcctaagtggagtaactcaggaatggaaaaaccaaatactgaatgttctcacttataaatgagagctaagctatgaggacacaaaaacatgcaaaatgacataatggactctaggggcttcttggtggaggttgggaatagggtgagggataaaagtcaacactgggtagagtacactgctcgggtgatggtgcaccaaaatctcagaattcactactaaagaactcacacacataaccaaaaactacctgtcccccaaaaactattgaaataaaaaaatttttttaaataaatgtggggttgatttgtaatttaaaatttattttgaatagctaatacaagaacaatatttaaaagattataataataaaaatgtgtatagtgtgcaacgcaaatttccttctccaccccatccacaattcttccaaattccttgcctggatgcaaccactgttaccagtttcagcaagaaattaagacagaaattaaagttctgctcctcataataatcacgatcaattatccctgcttatacagtaacccctccttttcccaacagcctcctagcatgaggaacccaaattgaacaggtaacaaacatacccctaaacgtaatgtaacttgtactctgttccatggtaggaacttccttctctgggatcccaaactcctagaccagctgagtacgttgaggggatgggaagcaccaacaccccacgcaggttaccggaagtgctggtgagtatagtcattccttcttccagcactggtggttagagacttactctacctgttggagacttggcaccatataacagctgctggcgtaaccttagtgtatgcttcatcctgaaggacattacccatcctgataggatgtgatcttcaagataaaacccagctgcaccctcaagagaccattccactgctctatcagctcgcagagtgtgtgatgcagtatggggtagaaccaggagatctcatggtcatgtgcccactaccagacatccttcgctataaagtaggtcccttagtctggggcaatgatatgtgggtcccctattagaagatcatacatatgtgagcccttagataatgtttctgagtgtggcactgtgggaagaaagaacaaactcataccgctaatacaaggcaacctaaatctgacagaacccctgcctttttcattgtgaaaaagtaagaatagaatcaatttgccatcaaattgctcattggcttccataagcgaaggtaatctgttggagtttatttatcgctggaaagtagattgtgacaataaccacgtcagccatgctgaaggcagcccaagccttgggacccacccacagtgcatctcttccaccatagataactgctttcaagagcctgtttcatgagcactgggatggttaaggacagaggctggctgatgtgtaccgaatgattcatcctggacacctggttgtttagactacttgtatagagggtaatttttgctgagtagtaacatctgatacaaaagatccaagcactttgtgtctactgtatgcctctttccagatattcctgttcctgatcttccaatcttgctccttccaggcccctgacctgctatttaagccacttgctgttgcctataagtctgtgcatattctcacctcaggccacttctgtctacacaaagtgggtgaatcagagccccacctgcctattggcaggaccctcttcaatgctgtctctactcattcctgagttgcccacactgcagacacactctactgctgcctcacaccaacacactaagctggcatgtttatgaagaagctcagaattttcctcttaagtcagctggtcatagaaactacccataaggtcattggtgagagtcgaggtcaaggcaaaggtccagcagtgcgaggtgacatggggtctgagcaaggtctttacgtaattaacaccagtactcagaacatacgtactttaacccaggggtcagccaatggtttcaataaaggcccagatggtttccctttgctgtgtgtgtgttcattcccctctccgaagctatgtgtaaccccatagagatcacaggggttcccatcaaccacagccagttacatccacaaaaaccttgatccttgttagcagatgggagagggtggcttggaggattgaaatccctacttcatgggactctggatacttacacagttgcttccttctgtgtgtctacatgaatctcagcactgtgtttgggacaccatctacactcttattcttcatgcaattctgtttgctcctcagatggccgcttttcctttgaagctcaacagtagccatcacgtacttgggctgagggttactgattaactctgtgggattctgggatcagttccttccctttcttagccatccctgtgctttacaaaacttagcttcacaattatactcatctctccctgtgtgcacctctaagaaattactgataaaacacgagtttttttgaaattacaaaaatatatgcaagaaaatgtttatggggccaattgatcagcacggaaaagcataaactttcctgttttctgcctctcattagaatttaatcccccaaaattgaactcattccaaacttctggcttaactaggagttgctgtagccagcctattctagacagcatcctgtaagcccctgggattggcagacacaagacactttttccattcatatatgtggttctgttctggccagatcagtggacttccagtgcccttcttgagtcatactgaggtaaattactcagaccacgaactcacatttttaaataaataaaatagcagcatagttaagaatttatttatcccattactgggtatatatgcaaaggattataaatcattctactataaagacacatgcacacatatgttcactgcagcactatttacaatagcaaagacttggaaccaatccaaatgcccatcaatgatagactggacaaagaaaatgtggcacatacacaccatggaatgttatgcagccataaaaaagaatgagttaatgtcctttgcagagacatggatgaagctggaaacaatcgttctcagcaaactaacacagaaacagaaaaccaaacaccgcatgttctcacttataagcaggagttgaacaatgagaacatgtggacacagggagggaaacatcacataccggggtctgtcaggggatgggggcaaggggtgggatagcattaggagaaatacccaatgcatgcggggcttaaaacctagatgacgggttgatggatgcagcaaaccaccatggcacatgtgtacctatgtaatgaacctgtacattctgcacatatatcccagaacttaaaagtataatttaaaaaaaagaatttatttcatttgtatannnnatgaaattttttggacattttttaaacctgcacttttcatagtttttgatacatccttgctccaataaataatttgctaaaattactaaatctcatagatataccaatggtttgcatcagctaatggcatatttgcttgtcaaactgaggtcagccagtcccccttcattcactgttagtaactcatgtgttgcattgttaaaaacccagtatcacctcatctcccctctagcatcttctctgcagaggagaagccaggcactatatctcccaggatccccttccctggatagttccagtttacattttccagtgagagagacttgcataagaaatggggccctggagagatggcgggacagacctgtacccatcagtggtggctgcaggtggaggagcaggcagatgtcaggttctcagtggcttccctgctaggctagagacccacctgctttgcttgtgcggactaagatgagtgataagagcttcctcataggttctggagaatatagcaatctcgcagcaggattctgagaacctcccatccatgcttcaagctgaagtcttcagtatatgcttccctgacctccctactgcagcttccaggagctccaatggtgactggtattattatagtatcccactgctgctataacaaactaccacaactatgtggcttaaaacaacaccaatgtattctcttatagttctggaggtaagaagtcaaaaatgggtcactatgggctaaaattaacatgtcgtcagagctgcatttcttctgaggctctaaaggaaaatctgtttcctcgccctttctagcttcttggggctgcccgcattctttggcttgtggtctcttcttccatcttccaagtcagcagtcacatcactttgacctctgattctgttatcacagctctaactctgatgctgctttctactcctttcacttataaggactattgtgattacatcgaacctgcccagataatccaggataatcttcccatctcaagagcctcagcttaatcacatctgaaaatttccttttgccctattaggagacatttttataagttccagggattaggacacagacatatttaggagaccgttattctgcctgtcacatgacttcaccaatatttgctacccgtggtttccaaacattagtgtatgctcttatttctatttattcctgaaataccagagtgagtctgtttttctgaaaacagctcactaatgcccagaggagctcaaaaattttctgtctgttgtctcaaagtctccaaattgtactgattcttcaatgtgcatggctgtaaattaggcaataatcttgctggggtgttggaaggagaagaaagggaatgattagacagcccttctgattcgggacagaatctgattcgggacactgtccctgatggctggccatcttgggctgtaatccagtttccctcccacgtagacccggtcatttctgactccccaagaggttttcacagtattcatgtccacttccttcatcagtcatcttcttgtcttatatctccatgtttacatttttcttctcttctaccactgcagttctgccttgattcttcagccattgccttccttttgcatgcatgtgcttcagccagttctctaggaagacattccagacagctcattatcacttgttactatgtttatttgactgcagagctttcaagatttgtaactttctattttttaaattttagtgtaaaaataattcttgctcacagtaggaaatgaaacaatcaaaatatgcataaatatacttttaaatctctcctttcactaaatccaaacccacttaactaaggtgattactattgtatcagcctactctctgcatgtctactttcttcacttgcacgcaaaccatagtgagtgtttttatctggtagcttttgttgagctgtaatgactataagttttatatgtaatttaatcattttcagggcaacttgagatataatttaaatagtataaaatccgcacattcaaaatatacaattcagtggcttttagaatattcacagttgtgcctccattaccacaatcaattacagatccttttatctcctcccagccctgcagaaaaatccatatccaaaccccagccctaagaaactaccaacctatgttttaactctacagatttacctattctgaacattttatgtaatgaaattatacaacatgtggctttttgtgtctgtcttcttttacttaccagaacattttcaaagtttgtgtgggtcgtagcttgtatcagtacttcacatctttttcatcgctgaattacattccattgtatggatatatcacaatttatttattagttggtgacattttggtccttctcatttggattgtgattaataatgctgctataaacatggcatataggtttttatgtgaactcatgttttcatttctcttgggtatactgtatcataggaggggattgctgggtcatgtaataacttaatgttcacaaatttgagctactgctagcctgctttctaaaggggctatgtctcttaaatccccaccagcaaagtgtgagtattctagtatctccacattctcatcaacactaattattatctttttaattatagccattctagtgggtgggaaatagtatctcatttttgggttgatgtgcatttccctgatggttaatgatgttgaacatgtttctatgtgcttatgggccttttgtatattatttgtcttattattattattattattattattattatttattgtttattattatcttattattaatattattatattgagttacaaccattctttatatattctagatacaagtcctttccttgccagatataagatttgcaaaacttttctcccattcaatgagtttcttttcactttcttgatggtttccttagactcacacatttttaattttgatgatgttcaatttatcttttatttcctctttttgcttgtgcctttggtatcatttaagatcaggtatttttacctaatccaaagtcatgaagatttatgcttatgttttcttctaagagttctctaatattaagtctcacatttaggtctttgatacactgaggtaatttttatatgtggtgtgaagtagggatccagctttattcttttgcatagggatagactgttgtttctgcactgtttgatgaaaagataatttcttcccctcactgaattatattagcactgttttataaaaccaattaaccatataagtgaagagtattgctgggccctcaattttcttctgacttcattctttttaatgctgttgtaagttggatttttaaataacatttgtattaatgcattgatactctatagaagcacactcaatttttgtattttgatcttgtagcctaccaatgtgctaaactaatttagtattagtggtttagtagttgctaagcttgtttttagtgaattctttcgtatttctatataagattttatcatctgcaattgagatagttttacctcttgctttttaatctagatgacttttatttcatcttattgctcattgtagcttttgaaaaattacattgtattcacagtgccctaccatttgatatttttatttctattatatcatgaacatcctttgaatttcacagaaaaaatggtttaactcatcattttaatgctgttccatagtatgttatatgcacctactcccctggggtaaatatttaggtggtttcatttatttgtcacccactctacagatgtctatatgcacatattctcccagccatcccagagttttctgatttcccataacgtcagtgattaaactcctgatctgtctgactagctagacatgctgactgactgacagttccattgctactttgcataatttaagatcaacagctacatttggatctcagatcatctaggcccagctgcagttagtctttcctagtctcagagattccctctctgcctgttaatttctctatccctgaaactgcccttgtactccagcccaagcagcttcctagcccagtagtcctggatctgccacttgctaactagaaggtgtcatctgcagaaggtaagtaaggcatttatctccattcactttatatttagttatcaaatctccatgtagctcaggtagatcgagctgaaaacattcttgtgtttagtaatatagccattgagcacaccatcattgctcttttcataccttagtcctactcaaagattaaagtgacagcttcccagttatgataaaaaagtataaaacattctgagggacctgtaaactataaagctaaaatatcagcttctgcatgcaacatatatgtcaaattctatgtccaatactcttctcatttttagaattaattctgcaaacttgaagattgagctttttatgaactgattggcatctgctatgctcaccagattattcccacttgactgagaggatggtggtagcaagagctccttcctttcagaggcttttaaaaatacttattgggaagaatgtaccacataaggcatttaactaatgaaaccacagatataaccactggcaaaaaaatcaaacaaacatttctttaactgccctgtacttggtacattctcttcctcctgctagggaagtatctcctctcccttcacttcacagactggaattttccaaattcctagtctcaacaagacaaactgtattttgaaatcactgttttcccagaatacccggagtgttaccatgactccagaagatgccttcaaccaagactcataatctgactgtaagaaaccttcagataaggtattgggaacttctgtaaaattgtagctgggctgtgattagagaattttcacaatatcaacgaatcttacaaaagagctgctgatccaacaggtcctcagagatcctgtgtgggaaccagaaggtattctgtgaaaaacgacaataagaataaccataataacaataatggctgcatttaaaaggctcttgctatatttcaggcactggtttttgtacctacatggattattttgttcaatatttatcacaaccatatgagaaggatgttattatccacaataaataaatgaaatctgtaaactgtaacttgttcaagaaaattaggtacagagatgttaagttacttgcccaaagttccataattttgaagatatgaggctagaattcagttccaggccattttacactagatctactaatttagccactgtatgattgtaccaagactgacctaaagctacctccccttccccaacaggttcttttatgacagtgagtataaaatacagggctaatgacgtattgggtgcagagaggggttggaaagaatgtggtatttcagactatgcttactatgcttaaaaatcacaattccgtggtgactagagtttatcttaaactttcctccctggcattccattgcatccagttgagagagcactctagctccctacatcccagaacatgtagatctgtccgtggttctgaaagacacactggcaggtcccgccaactacatgaatggcattcatattgtggaaaactgcccactagtaaaaatgtacagttgaatttgtacattcgttacttacaaggttggagaacgtgaagtactggacaatccaacaatagcttggaaagataccataaaaagacccgcagaatattcttttatatgcattattctgaaatgtacaaaacaaaaccagaatgggacacattatgagtgagaagtttatcttgaaattttaatcagaatatgaagaaaagaaatccaacattagacctggaaaaagataaaataatttgtatttcatttaacaatatttatagattctcaatatatacactatccttttaaacctcaatttatatttaaccatccatttacctttattttttgtttgtttttttctttcacctcatactttctacctgggatatctttttgtgactaaagtacattctttagaacttcttgtactttactaggtgtgtttgtgttaaattgtttgtcttcaaaattcattgagatttaaattttattattatattttcatgcttaagtgttatgtgtttgtctcttcaaatttttctgaagattatttatagttttatagtcactaaatgtatttaacaatctgttctttaatcatagcccatttagcaattttatatcctatacaattattctgatttctcagttatatgattgtatgattctgctgtctgttgtattttcttgttctttcacggtgctttgtctcttatgaacattgttctctgttgttgttcttaactgttaagtgctccttttgctggtgtaatgattttttggaattttcagagtgccaatgacagtagaaattttccagagaagttttgcttgattttccaaggttccttggagtctgcactactttaaaataaattcatggcttgatgattttcagaccacccagggagtgtgaactcgggctgcaaatccacttctctttaatctcagcagaaggttttgcccctctccactcagcaccaaggtgactttctagggagaccttcaagcaaggatgttttccttatactgaggagctggctttgggatcctggtgaaatgaggagaacactctattaaatttctcactctgagtgctacctgaactctgtcttctattccaaatgccttaggaggccatgaaaactaaaccttaattatcctacattaagtaaataagctcagcaaaaaaagtaaatgcacaattccgtgatttaggatttgacaatttttattaaagtttaagcctctcaatgcttctacattgtgttttattttttcaccatccatttttgttagcatgagggtaggtatgtttacctaaatgtgccatgtgagtggttcaaatctcctccccaacgctccttatcacaaaccttaccatcttattcctttaaagtcactgactatctaagcaaactactagctactgctgtgcattgattaggacccaacagtaagacgtttctccttttggtaaactgcacttccagatggtctaattgaacatcttctcactggaagtatttttatttccattactcttaatgatgattcctgaaagagactctaatgccctgacagttaactctggggtatccaagcatactcaaatttttttccttctacagtcaattgtccttagggaactcctcaagacctcaataatgagacaagggagagtggactctccagtggtaataagaacattaggagtatagtacaaattattaggcaacttacctggccttatggaacctgctaaagtctgatgctgtgtataccatttccccttgcagactgagggctggtgagcatgagtgacctgataaaactcaggtcataatgggcatctcaagtcacttggtcattttctgtatcatgatcatagaattcagaatctaccagggcccagtagtaagccagaagctattactcaaattcagaaaaagaatggaactttttccccaaaccctgaaacttgggcaatgagtcctatgactgccacacacaaacacacacacacacacacacacacacacacacacacacacatacactcaacaatatccaaataagcatttagggccaaatggcagatatgcttgacttttgcctggatcaaaatgacatttccatttattatggaatgtttctgtacttgtactagctcataggattttggattagagaaatacccttgcaggatatgaagtcatactttttcattggtgaaatcatctgttctcattagagtttaataatatcatgcagttatttctcaaagagcttgtactccttagatttgctggaattcctcaagttaaaaagaccagggcctcaataatgggaggtctcattgccaatagctacaactggctgtagaatttattaaaaacatgtgagtgcattgtttggggaaagaatcaagaacaaaagcagtgcttgttttgtacttcactgaactacttccatggcctccattcaactttagctgcagttttggtgacttgattcacagactgtcggaaagttatgagctatggaatgtgctctctccaatactaaagaggccagtcatatatatgtagggtgtccttctttatcattgtaggttgtatgaagtcagtgattcccatagaaccttggggtcacagatgccaggcctaatcttcttttattattcattcaatctttattttttcaaacaatggtctcactctcttgcccaggctggagtccaatggtgcgaacacaactcactgcagcctcgaccgccccaggcttaggtgatcctcccacctcagccttccaagtagccgggactacagctgtgtgccaccgcacctggctaatttttgtgttttttgtagagatgggtttttgtcatgttacccaggcttgtcttgaactcctgggctcaagcaatccacccaccttggcctcccaaagttgtgggattacagacatgagccactgcacccagctctaatcttattttaattttagaaatctaacttgacagtttctggtgcctggatccatatttgtctcagaagattgaattgtggactgtgaccttctctcccaaaagtctctttcagggtagaacttctggttatggcaacatgaagagatagaccagcaagttttcctccaacagcaacatgaaaactgtactaaatactaaaaacaaacatttgaagtcactggaaaattaacaaaggcaagcaatatgtgataagagctttcttgtttaagactccttctgtcttaggtaaggactgcaagttggtggcctttccttcttacctatgagtgctcttcagcctcccagcagataactgcagccctagtggctcaatatgggcagtttatagagttcagagcccaaatcagctagaaatgtaaatgagaatgttgaaagagagctgcagaagagttgagatcaacaatgtgaatgcaaattatgctcatccctagttgaacactaaactctgcatgggtgtgggccacttgggaatctggtgaaaaaccagaaagaaactagagggaggtctaccctccaaagagataaatgatcctggtgaggtctgcaagtcttcagtaacatggactgagtgcatttgccaacctgcacagagctcagtagaagaaagcaaaaatcttactggcttgagaggtgaaaaggcaggatttgaggctaacaaaacaattggaaattcaaagagaattccagaaacaaagcaaccacaatgaagttgaatcacaaaatctgagtataaatgacccaaatacttggtcaacctctaaattacacagatgcaggacacacacccagaaacccctgctgaaaccacgtaagatactaagtggaaattctcaaacagaaaaaaaagaaaatgaaagtattctggaagaggtccacagaagttttgagatggcagaataaagaacaagcctgtgcctttgaatataaatctccagaatgtgctcaattctaaaatccaaaacacagattgaaataagattaaagaaaaaaatgaagagaacgtcatgaacccatcagagaatataaagcaggtgaccagatatgcaagtggatcctcgaaagaagacacaggatcagaaaaaaatatttgagaaaataatagttgaaagtttcccacatttggtgggggattttagctcatatctaaggtgttcaataaatgaaagcaaaataactgcagagaaaattacaaatagggtgaccaaggccttggcctggcctgaggcaacttagctagccccagggatgtgtgttctggtgtggagtaagatcagggagctaccagtgcaatctctgcctctacatcagcgggactgatcattttaattttgattgcagttactgctggtatctgaatcaaagtggtgatgcttcctgaggaaatttctgaagccattagtcaattagaacattataagccatgcctcccgtgaagaggagcatctaatcccagcatttgcaccagggaactctgcctgaagggattaccagtgacttgaaagtgtaaccaacaacaatcttgtcactattacgcaccagctaagcaatctgaacatacatgctgaagacatatttggtgagctatttaaggaggctaactacttccacattagatcaaattctcttagaatttgttgcctcatggtcaaagtcactcagctggattcagcagtggaagaggtgtcaccacaggacatcaacatggaaaaagccctcaaaagttccccagcccaagaccagcaagcagtttcaaagagcagcattcctactcctgctgctgacatttacaatcagagtgattgaccacctcccctgaatagcatgagagcacatggagatgataaagacaggctgaagttctatactgatccttcccatttatttgacctctggaaagaagaagtgctgcaggacacagaagacaaaaggaaagacagaaggcatcaaaagtagcaaaaccacatagatggcaccacttatgatgtgaaaaggattacaaaagccagaaacaagcaccatgagtggaatatgatggcatatgacaaagagcttagacccagcaacaggttgtctcagtgtgtaccatggagcatcctgcaagaacccctgtccctagacagcaggtcccatacaatggatgttagggatgctcttatcgagctactccaaaccattctcacgacccttcctaggcagctgaagatgcatgtccacatgggcccacaaaccaggcccctgagcttgagtgccacccctcctctgcctcagtgaggcacatggccctaaatggacctcaagagcggcccccacttccctgccttaccccagctgtgttccaatcctgtgcacttatggctccaggagactacaggatgctcccagcacaggtgcttgcatattacaacccgtcaagacctccttctcttcccccttttacagtacaaactgcctttgttgccttctgcaaatcctcctgccgcctccttcctcatatcagctggctccacacaccctcctcctcaccctccctccactggcccctgggtccagcattccctcctccagccacacactttccccaccaggcctcttggctcagttcttcttttttgtcttccccatgcattcgcccccacactgccaccagtagctgaggcaaagtgtcaagaacctgcacagccaccaaaaagtgatacgagaagtgatctccttgctgctactggaatggggatttgactggaaaaggtgcaggatctgtgtgagcaagaggccaagagtgagccccttgggaaggacaaggtcacaaccctgtcaatacattgcagtggagaagtacagggattctgagggagacttagagtttgacaaaaacaactggcccaaccaaggcagtgctggggctagctctatgcaacaggtgggtagctggtaccaggctgggtgacagccaggagaccatatgctaagaatgtacttaagatttcaagtggtctcagcactcaatggtattataatcccgcagctgagcaccttttgtattaatgatgtgatattgcttctgcacatccaaaaattctgggctttctcagtatttactgtgtaacatttaaatgccattaagcatagaatatcatgctgcacatgaagaaataggatgtaactattacatcgtcctgaaaagagcgtttttttttttcctgtaggccatgaaagtatttagtgtattttgggtttactcttattgatcattacaattctgcagatgtgctgtgtgaagctgcctggtgctaggcctttgaagattaataactgggtttcaggtggatatcttcagatgagtaattgatattgttttactgttttggggtgttttatgagtttgaaaagctgttggttctagagggaaaggtttaacaaataaggagaatccaatatttctgctttcattttcttggcgtggtatcctctgtgtgaatttgaatgctcagttgaatagttttattgccttagtacacacttgtcaaatgtaaagttggaaggacccttttcaggaagggtttcctgtgaacattcacttagtaaatgcttgtggatgattcttgtaagaggtctcagtgcttagaagtaatgaaaatgaagaacaagatcctttcaccttcaggggactttgggaccaatactcaaaatatttgccagcccttacagcttgggcaccagccagcaggagcagaggtgagtggtccaggagctgggcagctctgggcagagcagctgcatgtgggccctgggtgctaaagcaggcctgctcattgtgtgtcctggaagagctgtgttctaaaggaggccacaggagttcttagctccttagtgtgtgacagacagtgcttcagagggcactgtgctccacttggtcttaattttgaaagtttacattgaggtgaaagtagatattgacagctgttcactcttcatgtgctgtgttcagattacggatgaatccatgagcagggcctttgccttatcctagaaacacgtggttatcttccctcatgttgttgctggcacatcacatgttgataagtacctctcaagattaccttgatagacccgtagctgtagaagagcacttagttctcacctctgcccttccccttattcttgcaccgtggtcatgctagagacccatggggtttattttcctggctaaggcaaactgttgggaaatgcaagaatgagagcttctattgtgtaattctggatgaagaataactgcttgtttagaacaaataaatgtcccaggctttgattatagttatgacttcaaaaatatgctaagaccatttacttactgaggaaatcttacctacttatagtatttcaaattgattgcagtcattagtgactttcagttacgatgaagggaagaatcttgagctaaacgcagggttgtctgaggtgatgataggagtggaaacaggatggtaactaatggatttaggttaatgagttctgagaaaatcaaccagcccctgcaagtcacagctctgtgtctctaagtggacacctgtctgtcttgaggcagtcatcatataatacgcatcacatagattttagtcatatggattttgaaactctggaaaccatatgtctgcagctccctttcatgaccatgcttgtgctggtttatctgatctcctcagctgtaccagctgctgtggcatgagcttcttgagcttctctttttagagctgatcattatctggcggctgtcgctataattttcttgaatacaagtaaacattatttataaacttttaaatttctctgtttaatttttctctctcttttttttttaatgtagaggcctactttgctggcatctttgggtcctactcagtcataaaccaccctccccaccctagagtagccactcttgatttctaatacagatgagttctgccaggtctagttgaatggcattatacagtcaccatcatgaccctgggtctcatcatctcgctgtgtgtggtcgtagcctgttcactcccatagctgtccaatgttccactttataaataactcacatttcttgtcctttcactgttgatagacaattttgttgtatacagattttacaaatcgtgctgctatgaataatattttcatatagtttggcacacacatacatgcatttctattggtttagaactaagagtgctattgctggatcataggtgagtagatgattggcttagtctctcaaaaaccaagtttctagtagtaaacaaacagtagaggagggtgaatctcttacttagaaattattcagataataaatgaagaaggaatgagagactgagactataataactcttttgcaacccatcatgaattaacagatttagccattgaatagcaatggcaattaacaacacaataaaagaaaaaactaatgtgaggttcttcctcttgatggaagaacacaatacagcaccatcaaacaagtccaagtgaaaaagaaaccaagcctgaatagaagctgatctccatagccaattaccaatttagaggcaatacagataatagaaatacatattaaactatgccttggggagcaatcagcaaaatgcacactatgggaagctctaccacacaatataaatttcaaggaagaatctatagaataaagacaacaaaagcatatttctaaaggtaaaactaaatcataatattggatgataaaaataaaaataaaacaaagaagtgatgatcgtaaaagtcaggttgtgtttttatttgagggaagaaagagtttattgctgggctggagcaaatgatggggcttctggtttgccccacaaacatctatccctggtgattaatgggtgtttactttgtattaaaggacacaaattttagacatttttttcttttggagtcctatattttattttatgacacaaaggctaatgaaaaaataatttatagagtatggttactgttttgcacaaagcctcctccccatcttcttttctggacactgagcacccagaacaaccagcagccccaggacccccagcaggggtgacctcacttctaggtgtgcatgcagcttacatcaccagaggtaacatgcaggtctactccttgactcctggaagaacctgataggacacagctgaagggaagccttctccgccatctgtaggctcttggccatcagtgtagagggagcgggtcctcacttctccacaggcgtcttcatggccatagcctcctctctgcatggggagtaaggcctggcccttcgcctgaacacggtgacagggatctcgccaaaggtggagatcacaccattcctcctttagcaacctcacgcttcttttgctttacaggaaagttgactcaggctggtgtccagtaaagaaatcccaaggagaggccgggtgcggtggctcacgcctgtaatcccaggactttgggaggtcgaggcgggcggatcacgaggtcaggagattgagaccatcctggctaacacggtgaaaccccgtctctactaaaaaatacaaaacattagcagggcgtggtggcgggcacctgtagtcccagctactcggaaggctgagccagaatggcgtgaacccgggaggcggagcttgcagtgctcctgctcccaggaggcggatgcccaggatgaactttaaggtggagagaggaatgtcattgctcatcctccaggttccaagtaagaaccttcctgcccagcccatctcgatctccctgaatcctcaacgctaataaggactgtctccttctcacctccctagactgggctttttaacactggagagtggatgtgatttctagtttcagcatgtcctggtttattgtgctgccagtaaaataaaatcaaaatacacattgaataaataataaataacccatagtgagcaaaggcttacaatgtatttttttgagtcaagacctcactctggcctgggctggagcccagtggtgggatcgtagctcaatgcagccttgtatttatgagctcaagggatcttcccacctcagcctccacaatacctggaaatacaagcgcccaccaccaggcccagctgtttacttatttttatttttttagagacgagggtctcactatgttgtccaagctggtctctaattcctgatctcaagcaatcctcttgccttagcatcctgaattgctgggattacaggcatgagctgtcatgcccagcttgtaatgtactgacactatcaaggtttctttctccaagtgtggacacagcagcagacaccccttgtctcttgggtcaggacactgggtagagtggaagagcagggcaacaaagtcactgcagaaagcacccacgtggaagaggtccagcagggagagccacctgttccagggacaccatatttagggataactcctctttctgggcaggactgttctttgattacttttgtattcacaatagttctgaaatcgtaggatgatgagactcaagactggctagggttttattttggtttagtttttattttgttttgttttgtttttttcaaaaaaccatcatgagatttgttaatcttttgaatggtttttgtgtctcaatttccttcagaacttaactcttcttaacatttcacagtccactttcagttaaaaatgtactagtacccataaatagagaaatcttgcaaccatttatcctctataaaatgtccatctttatggtacagatattatatgtaatatatctatataggttataagtcccacaagaaaaaaacttcttcttatctttaaacagttctatgtatataaattagagtaaatgaggggaacaagatagccttttgcacttagctcagtatttaccatttttgatcatcttcatcttttctgataacctcatttcccacctggtatcatttgcctttagcctgaagaattttctttaccaattgttattgtgcagatctgctggtgacaaattctcttaattttcttttacctgaaacatctccttattttgcctgtccttgaaggacatctgtgctggatatagaattcttagttgtcctttttttcttccagcactttaaaggtgttattctactttcttttgtttctatggtttctaataagaagtcattggtcaggacaagaggaacagagaaaccaaaactgaggaaataacatggtagacctaaaacttatcaataatgaaaaaaaaacctatatgaatcaacactccaatgaagggcagagattgtgtgaattggttaaaaaaaaaaacaaaacacagaacccaaccatatgctctttccagatatgcagtttctttgtttcttttttttttaatttaacttttatttttagttaaggagtacatgtacaggtttgttatataggtaaacttgtgtcatgcggttttattgtacagattatttctgtacccattagtacccacccaagtattaagccttagtacccattagctatttttcctgatcctctccctcctcctctaccctcaagtggaccccagtgtgtgttgttcccttctgcgtgtccatgtgttctcatcatataactcctacttataagtgagaacatgtggtaattggttttctgttcctgcctcagtttgctaaggataatggcctccaactccatccatgttcctgcaaaggacatgatctcattcttttttatggctgcatggtattctgtggtgtatatgtgccacattttctttttcttttttttttaatggaaaataacttttattgagatcccaccagttgcaatatctgttcccagcattaagctccttcttcctttgcaattcggtctttcttgagtggtcccatgaatgctttcttctcctccgtggtctggaagcggccatggccaaacttggagggggtgtcaatgaacttaaggtcaatcttctccagagcccgctgctttgtctgcaccagcaaggacttgtggagggtgagcacccacttcttggttcccaccacacagcctttcagcatgacaaagtcattggtcacttcaccatagtggacgaagccacccaaagggttgatgctcttgtcagacaggtcatagtcagtggaggcattgttcttgatcagcttgccatccttgataaggtagccctagccaatcctatagatcttgttgatctcagtgcggtgatggtagcctttctgcccaccacgtaccacagagaagcccacacgagcaggatgccatgccccaatacaggccaccttgcacaggccttggtgggtcttgcggggcagcttcttggtgtgccaacgactggtgacccttttgtagcctttgcccttggtcaccctgatgacgtcgatcatctcatcctgcccaaacacttggttcacaggtacctggtgcttgagcctctcgccagcccagtccagcttctcagccacagtgcctccattcacctggatctccatcaggtgggccttcttctggcacagaggaagcaggtgcatctgggtgtgggcaatgacgcagatgacttggcagtacttcttcatgctgctgaagtccttctccagctgcttcttgccatcctcatcctgccatttcttgcagtacttggtaaaggccttcttcttagatttatgccagttcttatagaaacgcctcttgcattcatcactgatgtgctcagcgaagacagtcttgcaagtccggaagccttgaggggtttccacgcagcccacaatgcccactggtggcctctccacaatggtcacagcctccaccacctccttcttgttcaccttggatcctggcctgtcgacttcccgcacgatgtgggtcatgccagccttgtatcccaggaaggctgtgaggtggaccggcttggatgggtcatccttagggaagctcttcaccttcccacgatgcctgctgctgcgcttccaaggcaggaagccgagggacccatgtctgggagtggagaactttctgtgagacatcacgccatcaaatcctgccggtagagcatatttgccacattttctttatccagtctaccactgatggacatttaggttgattccatgtctttgctattgtgaatagtgctgcaatgaacatatgcatacgtgtaagacatgggctttctttttttaatttgctttgttttattttactttaggttccaggatacatgtgcagaacctgcaggtttgttacatacgtatagtctgccatggtggtttgctgcacctattgacccatcctctaagttcccgtcccttgccccccacactccccaaaaggccctgatatgtgttgttcccctccctgtgtccatgtgttgtcattgttcaactctcacttaggaatgagaacatgaggtgctttgttttctgttcccgtgttagtttgctgaggatgatgacttccagcttcatccatgtccctgcaaaggacatgaactcattcctttttctggctgtgtactattccatggtggtatgcaccacattttctttatccatctatcactgatggacatttgggttggttccaagtctttgctattgtaaatagtgctgcagtaaacatacgtgtgcatgtgtctttatagtagaatgatttatattcctttgggtagatacccaaagcgtcaaatagtatttctggttctcgatctctgaggaatggccacactgtcttccacaatggttgaactagtttacattcccaacaacagtgtaaaagtgttcctatttctccacagcctcaccagcatctattgtttcttgactttttaataattaccattctgactggcgggagatggtatctcattgtggctttgatttacatttctctaatgagcagtgatattgagcttttttaatttttctcattaagattttaatgtctcattttaaaaaattaaaggaagtttccatttattttttaagataaagatttagcgcacccaaatgcccccaaagccaacagaaaaatagctttgccctgtcatttccctaagaaagcactgcagttactcaaaatatgctcaaagaaaaaaatgcaatcctctgagttctaagtttcacgaaaggaccacatgttaaactatgtatatcgatttgatgtgtaagtatgcaataaatatgtacacatacattcctatctgcttcacatcattctagagtattcatagtatatgaagatgggatttagaaatgtgaaaaggctgtaacagtgaaaaggaaaaaaagggtacaatggtttttagaccagtagaataataatgcttagctagttaattattttaactttggagcagactaaaaaagttgttttgaaataaatggtacctgtaagtgatgcttactgcaatacttgttaagtgttatttgtcaccactgacaggaattatatagtgctagtaacacagctcccactgtcctagaattcctctaaatggccagctattgctaccattatcttgataagcatttcaagaattgtgttgctgagccttttaaaagataactagtttataacctatgtctccttatgtgcctacatcagttactcatggtcccaaagaagtataaaagaggatataagctgctgaagatttcacagaagaaacagagatatcagaaacttcatgatcatcaaacttaaaccaccgctgttttgctgcatttttacagtaggctgtgtaatggcctccatccagcccaccgcagtgatctgaaacagaaaacaaattatatttcttcaaattgttctttggaccaataacatactgtgacaaggcaagattttctaacaggaagtccacagatgtctgtaatttttgtttccacctgccattgtaggaaaaatgtttcagatgcactaaaagcacaggtggtaacttccagatttcttttttaagaatcccgtcgagctctgcaaagattgcagtaaaatctgttattatctatgagtttttcttctttagaaaataatctaaggcaatcctataatgtacatttacttgtggatgctatcagtagagacaaatacatgaaggcctcaagtgtcctagactttttgtgacgggtgaggcactgtactgtagatttgaattgaccctgaaaaagtgcaacaataatagactcatagagccgcttgtgtttctgccaggcatgttctgcagctttaaagtcattgagatgatcattattttcttctttatatgtcttccgattatcagttttatttagatcttcatggagaccatccattaggaacagaagaaattcttgtgaatcttgactgtatcctgcaaactggtaattgatcttcccaatggtgactttaaggtcttttggactgatatatctatactgtcctgtccacggggctttcatgattataccaaattcttctgccacttcacctttatgcccctaacaaatttgacttgttaatatcatcctgataacagtttcggttgaaataatcagccaaatgtggagcattacataggcactgcaatattgagttcatataacaagtatttcctaagttacgaagtccagtaagagctggtccagaacctccaaaaacaggattgagtttccaaatctgagaagcagaaagccttgagatctcagctttagggtagcatgttggcttgtcttcctgattaactgttggagttactgctggcttcctcttgaatagcctgggttatatctggggaggagtaagagcatttcagtttggaaggttccctatcctgcttagcaggaatctgtggcttgcctttatgagttggaggggtggaaggaggtgcagatgaaggagccatttccagtgggtacatatgaacagtgttagtggtgaatgataataatgaaacgttccagtgattggatcaagaaacttggcccagcctgaaggcagtcctggtaccatcctccccatttcttcacttcgtgctattatcgaaggttctcgttgagttttatttctttcagtatcatctgtatcctctctaaaagtttctgtccttagaattccagtttctggttgtcctttaatcttaaaaggcttgcctgaacctgaatcccctgtcgcagatgtatgggacacatctcttgttgactttttctgtacttctggggttgaacatcttctccccctgtcctctacagacttctcggcaccagaggtttcatgatcacttttactttgtctttttactcttgttatttctttgcccttctttgctgagatttctttatcctctttcttggcctgttcacgttctttcttctccatttcttcttttgctttttgttgcttctgtgtaatttcattttcttcagcttcttgtttctttttggctttttgctcgtgttcttccctcctcagtttctttctgtttcccttgctgtctttcctgaagttctttttcttgtttgtttttctccattagaagagcagtttctgcatgaatatgagccttttcttcatctgttaacatgagagttggagaggaaactactggcttggcggaacaatcaggaataactttttcattctgaggagactgttgctcatgatttgtactttcagatttgattatatgctcttcaggcaatttgactgccagttttttagtatgatcaatctgtggaacattctttatgctaggcactggctgaatgatgggtgaaacatcagatttagaagcagcaactgattcaactggaattgatatattcagtggtcctgtcctctcattttgatcattatcacttatttgatcacttatcaattctatgtcttcatccacttctataggtggaggtggcatctcggcagcaggtttagaaggaattgattcttccaatgagggataagtaaaatccaatgagatagacaactcttcattctggtgttgtgggggtggagtgaccttagcatttgttgtatactgggaatagcaaaggaaccagtttttatagcctccctctaaaaccaaaggcccaaggcccattgcacaggacagttttactttcccacttgaaaagtgcatctttcagatgccagagtgttgttccaatctgtaaatcttttgcagaactaaaccagtcaagaagtaccatatactccacattccccctcttcttccatgtatctatagaatcatctgggaggtgtgcttcaatccagctagcagtgactcctggactgatggcttttccaggaacactgagagaatgtaaaatacaggaatcctgataatcctgcattctttgagcatccattataatcaagctgatgtttttatccatcatcattgtgtatagttcctttgctgtgattgctcctttctctttggtctcacatttttcattcttttcaccattgctcttttgggttttgtctttggaatccaatacaatctccaaagaatctttagccaacgtgctgccatcctctcttcctgtttcctgccttttttgttgtagccactgtgcttccccctgcctgtccttttcctcaagttttttccagatttcaacttcttcatatcttagtttaaggctttcagagagtcgttcagtttctccagtggcttttttgatgtttgcaggtccaagtattgaatggaagtaatactgctgttgcttgaaatcagttcttttttggataggattataaacagtcatgtttcatatacatgtgtgagctttttttcatatgtttcttggccacgtaaatgtcttcctctgagaagtgtctgtttatatcctttgcccactttttgatggggatgggtttttttcttgtaaatttgtttaagttccttgtaaacaaacatgtgagctctcatcattcttgtttaaacacctaacagtcatcctaaccagtgcaacaaggaccaggcatggtggctcatgcctgtaatccctgcattttgggaggctgaggtgggaggatcacttgagatcaggagtttgagaccagcctgataagtgagacctcatctctaccaaataaaaataattttaaaagaaaatagatcaatggataagaaaggaagaaatgaaagtctttctttgtcaccagcttcattgtatatgtagaaaacactagggaattctgaaaaagtctctggaattaatcattgaatttgcaaaatagttcataaaatatatgtaataagtcgcttagatgaacatgaaaagatagcaaacaatactagtcatcaaagaagtgcaagttaaaaccacaatgagaaaccatcacacatcacctagagcaggtaaagttaaaaagacatatgataagtcttaacactggcaagaatatggaaaaaataggaatgtctgatattgctggtaggaatgcaaaaaatgtggcagccaatttgtaaagtggtatggcagtttcttatacagttacccatctattaccacatggcccagcaattccacaaatacgtatttatccaaaagaaataaaaatgtaagtccacacttgtaagcagttatttatagtggcttcattaataacaagccctaactggaggaatccacatgtctatcaactggagacacagaaaccaatgaataaactgggattccagcaatactcggcagctgctcagcaacaaaaatgaatgaatggcatcatctcaaacatcgttatgctaagggagagaccaaacaaaagactacataacatatgattgcatgtccatgaaattctagaaatgtcactattgcagtgacagaaagcacagcagtggttgaatgaagggaagggggtgagggtgggaggcaaggattaaatagaaaggggcataaagaaagtttttagggaaaagaaactgtcctctacagcgccacaactcaggagtgactgggagggggaagtaaggggagaagaaggtctgagggataaggggcagagagaagggctggggaagcaggaggagaggacgaggagcaggggaaaggactctaaagcagtggaggggcctagtaggaggatctttgcatttggtgtttctctactgggcagtgtggtagttacactataaataattaccaatatccaccaaaaagtgcagctaaaactggtgaatttttttacacgtaaacgccctgataagcaaaaaaaaaaaaaaaaaaaaaaaaaaaaaaaaaaaaaaagggggggggggaggaaagaaggcaaaaataaagacattgttagatcatcaaacccataaaattcatttcctaggggtcctgtactacatgtaattttaaaggacgttctacaggctgaagggaaatgatactagatggtgacccagatacacagaaaggaacaattaacaacagaaatgatgcacatacacatatcacatacacacccattttcttaatttcctgaagatacgtgactgcttgtctaaaacaaaaagtattacactgtattgttgagtttataacgtatattgatgtaacatatacaataataatagcataatgatagtttaaatgaaactacaccatttgaagtgtcctttattttgctggatgcagcttaatattacctgaatttcactgtgaaaagtcaaggaattgggtttcaattcttacaacaataaaaaattaatgtaaagaaatatagctaaaagccactaggagaattaaaaccataagctaaaaaatgtttacttgaaacataagaaagtagcaaaggaggaacagaaacaaaaagatatgagacaaattgaaaacgtatagcaaaatggtagaccaaaatccaaccattataagtgaagaaatgacacgacctgagtcacattagcaggactgctgagcactatggggagaacagacatgggcaggaagtgagggacagtgttagtgccacaattcaggagtgacagggtggcggggactaaaggggaaagagggtgtgagggatgagaggggcagacagaagggctggagaggcaggaggtgaggaaaaggagcagaggaaagaattctaaagcagtagaagagcctggcagggggttctttgcattcggtatttaatacattttgtgtgactgccttaaaactaatgggctccttatgatttttttttaaaaaggggttacaaaaatatcaagtgtccaaataaaatatgcacactgcttagatgtgcatagttccggaaaacgggcagtgctggagcgctggtgaagagcattgggactgcatggagccctcacaactttgaggtgatgactacaggctcccggttgcaatagacagtaacaaaccctgcttctttgtattcaggagatgttctggactcacacagggaaactctggctagagaatgaggataactttaaatgcaacaacccagagtcacagatccatagtctgcgaaagtaaaacaggagctttgagaatttaattgtaatgcagttttgacacaggtctttcacagattggaattctaatcattcagggattaccaatattgtgctacctactgtatcaataaacaaaaaggaaactggtctctatgagaatctctacctggtgctttcagacaaaacttcaccaggtttaaagagaaaactcctgactctacacgtccattcacagggcgagctcactgtctggcatcaagttccccatggtgagtttccctgtacaagagtccaaggggagaggtaagtgtcctttattttgctggatgtagtttaatattacctgaggtaaggtaaggcaaagagtgggaggcagggagtccagttcagggacggggattccaggagaagtgaaggggaaggggctgggcgcagcctgggggtctctccctggtttccacagacagatccttggccaggactcaggcacacagtgtgacaaagatgcttggtgtaggagaagaggaatcaggacgaagtcccaggtcccgggcggggctctcagggtctcaggctccaagggccgtgtctgcaatggggaggcgccgcgttgaggattctccactcccctgagtttcacttcttctctcccaacctgcgtcgggtccttcttcctgaatactcatgacgcgtccccaattcccactcccattgggtgtcggattctagagaagccaatcagcgtctccgcagtcccggttctgaagtccccagtcacccacccggactcagattctccccagacgccgagatgcgggtcatggcgccccgaaccctcatcctgctgctctcgggagccctggccctgaccgagacctgggccggtgagtgcggggttgggagggaatcggcctctgcggagaggagcgaggggcccgcccggcgagggcgcaggacccggggagcagcgcagggaggagggtcgggcgggtctcagcccctcctcgcccccaggctcccactccatgaggtatttctacaccgctgtgtcccggcccggccgcggggagccccacttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccagtgagtgaccccggcccggggcgcaggtcacgacccctcctcatcccccacggacggcccgggtcgccccaagtctcccggtctgagatccaccccgaggctgcggaacccgcccagaccctcgaccggagagagccccagtcacctttacccggtttcattttcagtttaggccaaaatccccgcgggttggtcggggcggggcggggctcgggggacggggctgaccgcgggggcggggccagggtctcacatcatccagaggatgtatggctgcgacgtggggcccgacgggcgcctcctccgcgggtatgaccagtccgcctacgacggcaaggattacatcgccctgaacgaggatctgcgctcctggaccgccgcggacacggcggctcagatcacccagcgcaagtgggaggcggcccgtgaggcggagcagctgagagcctacctggagggcctgtgcgtggagtggctccgcagatacctgaagaatgggaaggagacgctgcagcgcgcgggtaccaggggcagtggggagccttccccatctcctgtagatctcccgggatggcctcccacgaggaggggaggaaaatgggatcagcgctagaatatcgccctcccttgaatggagaatgggatgagttttcctgagtttcctctgagggccccctctgctctctaggacaattaagggatgaagtccttgaggaaatggaggggaagacagtccctagaatactgatcaggggtcccctttgaccactttgaccactgcagcagctgtggtcaggctgctgacctttctctcaggccttgttctctgcctcacgctcaatgtgtttgaaggtttgattccagcttttctgagtccttcggcctccactcaggtcaggaccagaagtcgctgttcctccctcagagactagaactttccaatgaataggagattatcccaggtgcctgtgtccaggctggcgtctgggttctgtgcccccttccccaccccaggtgtcctgtccgttctcaggatggtcacatgggcgctgttggagtgtcgcaagagagatacaaagtgtctgaattttctgactcttcccgtcagaacacccaaagacacacgtgacccaccatcccgtctctgaccatgaggccaccctgaggtgctgggccctgggcttctaccctgcggagatcacactgacctggcagtgggatggggaggaccaaactcaggacactgagcttgtggagaccaggccagcaggagatggaaccttccagaagtgggcagctgtggtggtgccttctggagaagagcagagatacacgtgccatgtgcagcacgaggggctgccggagcccctcaccctgagatggggtaaggagggggatgaggggtgatgtgtcttctcagggaaagcagaagtcctggagcccttcagccaggtcagggctgaggcttgggggtcagggcccctcaccttcccctcctttcccagagccgtcttcccagcccaccatccccatcgtgggcatcgttgctggcctggctgtcctggctgtcctagctgtcctaggagctgtggtggctgttgtgatgtgtaggaggaagagctcaggtagggaaggggtgaggagtggggtctgggttttcttgttccactgggagtttcaagccccaggtagaagtgtgccccacctcgttactggaagcaccatccacacatgggccatcccagcctgggaccctgtgtgccagcacttactctgttgtgaagcacatgacaatgaaggacagatgtatcaccttgatgattatggtgttggggtccttgattccagcattcatgagtcaggggaaggtccctgctaaggacagaccttaggagggcagttgctccagaacccacagctgctttccccgtgtttcctgatcctgccctgggtctgcagtcatagttctggaaacttctcttgggtccaagactaggaggttcccctaagatcgcatggccctgactcctccctgtcccctcacagggcattttcttcccacaggtggaaaaggagggagctgctctcaggctgcgtgtaagtgatggcggtgggcgtgtggaggagctcacccaccccataattcctcttgtcccacatctcctgcgggctctgaccaggtctttttttttgttctaccccagccagcaacagtgcccagggctctgatgagtctctcatcgcttgtaaaggtgagattctggggagctgaagtggtcgggggtggggcagagggaaaaggcctaggtaatggggatcctttgattgggacgtttcgaatgtgtggtgagctgttcagagtgtgatcacttaccatgactgacctgaatttgttcatgactattgtgttctgtagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggacagacagcccacccccgtgtccaccgtgacccctgtccccacactgacctgtgttccctccccgatcatctttcctgttccagagaagtgggctggatgtctccatctctgtctcaacttcatggtgcgctgagctgcaacttcttacttccctaatgaagttaggaacctgaatataaatttgttttctcaaatatttgctatgaagggttgatggattaattaaataagtcaattcctggaagttgagagagcaaataaagacctgagaactttccagaatccgcatgttctctgtgctgagtctgttgcaggtgggggtggggaaggctgtgaggagccgagtgtggacgggggcctgtgcctagttgctgttcagttcttcatgggctttatgtagtcagtccttagctgggtcaccttcactgctccattgtccttgtcccttcagtggaaacttgtccagcaggagctgtgaccacagaggctcacacatcgcccagggcagcccctgcacacgggagtccctgtgctttctgagacaaattttcagacccattcagctcctgccctccttatagggctcctcttctgctttggtctcctgccctctctcccttccctgattccagtgatcttcgtgctgactccaatcccaactcatgaatctaaagcagagcctaatttagatttgtatttgtttgtaaaattgggtccatagtctagaattgttccttcctgaagagagaaacctgatcgtgtgctgcagcgtgcagggcggttggtgtgggaggagggataggggagggaggacacacaagcagccctgctgagaaaagtacaggcggcctcggtgtcagtgtgaggggaccttgtgctgcagctgccacaaaacagcacttggcctgaggctatgttaataaagatactggctttagaataggaggtgctctacactgatcattcaactgacctttgttgtcagccagacacaggacagaaaagttctgcatctggggaacaccattgaagtaaaatcagaaaaatatctgagcatatgctccagtggtaagaggcagacgatacatacactataaccacagtaagaaaagaaagtgatggaaggtggtaagtgccatgaggcaggtgatccgggtatgggcagtggggacagggaaggtggctgttggacaggagttgtcaatgtgtgccttgttgcaaagatgacctttgaggaaagatttgagggacatgaggatgtctggggaagttctttctaggcaaggaaacacaacttcgtatagaaaggtatatacgaagttgtgcggccgcgtcgaccaattctcatgtttgacagcttatcatcgaatttctgccattcatccgcttattatcacttattcaggcgtagcaaccaggcgtttaagggcaccaataactgccttaaaaaaattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtgaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggac

SEQ ID NO: 15 - DNA, 7695 bp, synthetic construct
  source 1..7695: mol_type = other DNA
ttattatcatgacattaacctataaaaataggcgtatcacgaggccctttgtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtggtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgcgagaccttccgggtctcagaagttcctatacttcaaaaggaataggaacttcggatccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccgatcatcaagcttgatcctcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtcgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagtcgactaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgggaagttcctatacttcaaaaggaataggaacttcttgtagagaccaaccgggtctcagctagctccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaacca

SEQ ID NO: 16 - DNA, 14161 bp, synthetic construct; identical to SEQ ID NO: 11
  source 1..14161: mol_type = other DNA

(19) United States
(12) Patent Application Publication
(10) Pub. No.: US 2023/0295668 A1
(43) Pub. Date: Sep. 21, 2023

(54) METHODS AND COMPOSITIONS FOR INTEGRATION OF A DNA CONSTRUCT

(71) Applicants: Opentrons Labworks Inc., Brooklyn, NY (US); New York University, New York, NY (US)

(72) Inventor: David Minh TRUONG, Brooklyn, NY (US)

(21) Appl. No.: 18/185,601

(22) Filed: Mar. 17, 2023

Related U.S. Application Data
(60) Provisional application No. 63/321,561, filed on Mar. 18, 2022.
(60) Provisional application No. 63/322,664, filed on Mar. 23, 2022.

(51) Int. Cl. C12N 15/90; C12N 15/11; C12N 9/22

(52) U.S. Cl. CPC C12N 15/907; C12N 15/111; C12N 9/22; C12N 2310/20; C12N 2800/80

(57) ABSTRACT

Provided herein are methods and compositions for integrating a large DNA construct into a genome in a cell. Also provided herein are methods and compositions for producing a synthetic genome.

### CROSS-REFERENCE

This application claims the benefit of U.S. Provisional Application No. 63/321,561 filed on Mar. 18, 2022, and U.S. Provisional Application No. 63/322,664 filed on Mar. 23, 2022, each of which is incorporated by reference herein in its entirety.

### STATEMENT AS TO FEDERALLY SPONSORED RESEARCH

This invention was developed with support from the Government of the United States of America under AI154417 and AI148008 awards by the National Institutes of Health. The Government of the United States of America may have certain rights in this invention.

### SEQUENCE LISTING

The instant application contains a Sequence Listing which has been submitted electronically in XML format and is hereby incorporated by reference in its entirety. Said XML copy, created on Mar. 15, 2023, is named 59725-710_201_SL.xml and is 389,788 bytes in size.

### BACKGROUND

Manipulation of genomes such as genome editing or rewriting is an important technology in developing therapeutics such as gene therapy, cell immunotherapy (e.g., T cells, CAR-T cells, etc), and regenerative medicine (e.g., induced pluripotent stem cells) to treat diseases such as cancer, autoimmunity, and neurodegeneration, or for tissue/organ replacement and rejuvenation. There are many challenges and limited approaches for manipulating genomes or site-specifically integrating large DNA constructs into human and other mammalian cells. For example, there are no straightforward approaches for integrating large (sub-megabase) constructs sequentially, to allow for megabase scale DNA integrations, e.g., placing the large DNA constructs anywhere in the genome or in any cell types. In addition, there are limited approaches for providing “scarless” or “nearly scarless” large DNA integrations, i.e., no remnants of unwanted DNA such as selectable markers. The ability to integrate large DNA constructs into the human genome, especially in human iPSCs, would facilitate the rewriting of the genome, delivery of large genetic circuits, and provide a platform for producing cellular therapies and regenerative medicines.

Cellular therapies hold great promise for combating previously intractable diseases (e.g., cancer, organ failure, neuropathy, etc.). While autologous (patient-derived) cells generally are optimal, this route remains limited for many applications, cost-prohibitive, and burdensome. Allogeneic (donor-derived) pluripotent stem cells (PSCs), which can be differentiated to any cell type, provide greater scalability and cost savings. However, the allogeneic approach is limited by Human Leukocyte Antigen (HLA) immune-matching, as without HLA immune-matching, the transplanted donor cells will be rejected by a patient's immune system. There is a need for developing a technology to provide personalized stem cells for both immunotherapy and regenerative medicine applications, leading to safer and more efficacious cell-based treatments.

### SUMMARY

In some aspects, provided herein is a method of integrating a DNA construct into a genome, the method comprising: a) contacting the genome in a sample with one or more agents, wherein the one or more agents are capable of cleaving the genome at a locus; b) integrating a first nucleic acid sequence into the genome at the locus; and c) integrating the one or more second nucleic acid sequences into the first nucleic acid sequence in the genome, thereby integrating the DNA construct into the genomes, wherein each of the one or more second nucleic acid sequences comprises a cargo sequence.

In some aspects, provided herein is a method of producing a synthetic genome, the method comprising: a) obtaining a cell from a sample, wherein the cell comprises a genome; b) contacting a first nucleic acid sequence at a first locus on the genome with a first set of one or more agents, wherein the first nucleic acid sequence encodes a first plurality of antigens and wherein the first locus comprises one or more binding sites for a first set one or more agents; and c) contacting a second nucleic acid sequence at a second locus on the genome with a second set of one or more agents, wherein the second nucleic acid sequence encodes a second antigen, and wherein the second locus comprises one or more binding sites for a second set of one or more agents, thereby producing the synthetic genome.

In some aspect, provided herein, is a method of producing a synthetic genome, the method comprising: a) obtaining a cell from a sample, wherein the cell comprises a genome; b) contacting a nucleic acid sequence encoding an antigen at a locus on the genome with a set of one or more agents, wherein the locus comprises one or more binding sites for the set of the one or more agents; c) integrating a synthetic sequence to the locus on the genome, wherein the integrating the synthetic sequence to the locus on the genome preserves regulatory components for gene expression, thereby producing the synthetic genome.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic of sub-megabase-scale genome writing design. First, a landing pad sequence 105 is integrated into or in place of a locus of interest in a genome 101 in a cell derived from a sample 130 with one or more agents 120. Then, a DNA construct 102 comprising one or more cargo sequences 110, 111, 112 and a selective marker 125 is prepared and delivered to the cell 130. Next, the DNA construct 102 is integrated into the landing pad sequence 105 at the locus of interest in the genome 101. This step can be repeated to integrate multiple DNA constructs sequentially to the megabase-scale.

FIG. 2A depicts an exemplary diagram of the REcombinase WRiting of Iterative DNA and Trap Excision (REWRITE) for landing pad sequence (LPneo) for a single (sub-megabase) large DNA payload delivery.

FIG. 2B depicts an exemplary diagram for a configuration of the REWRITE landing pad sequence (LPneo) for iterative large DNA payload delivery to the megabase-scale.

FIG. 3A depicts a Landing Pad 1 (LP1) plasmid schematic with figure legend. Plasmid backbone markers (ampR, ura3, and CEN) enable propagation in both E. coli and budding yeast for recombineering.

FIG. 3B depicts a diagram of LP1 integration into the murine ROSA26 locus. ROSA26 is a ncRNA flanked by the genes Setdb5 and Thumpd3. Homology arms to ROSA26 were cloned via recombineering into LP1 and integrated using a CRISPR/Cas9 gRNA.

FIG. 3C depicts a Polymerase Chain Reaction (PCR) verification of 5′ and 3′ junctions (jxn) of Clone #2 and Clone #4. LP: landing pad.

FIG. 3D depicts a Polymerase Chain Reaction (PCR) verification of the 3′ junction (jxn) of an integrated 10 kb (HLA-A) payload delivery into the landing pad of the same in FIG. 3C. the left lanes are negative controls. C: Clone.

FIG. 3E depicts a Polymerase Chain Reaction (PCR) verification of the 5′ junction (jxn) of an integrated 10 kb (HLA-A) payload delivery into the landing pad of the same in FIG. 3C. the left lanes are negative controls. C: Clone.

FIG. 3F depicts a demonstration of the “Flp-OUT” component of REWRITE to provide scarless insertions, where Flp recombinase is induced after Payload delivery. Induction of Flp recombinase (using 4-OHT, 4-hydroxytamoxifin) from within the payload excises the gene cassettes via FRT recombinase-mediated excision. Because the cassettes express Thymidine Kinase, only full excision is resistant to Ganciclovir (GCV), whereas those not excising die off. Stem cell clones are shown after payload delivery in FIGS. 3B-D, induction of 4-OHT, and selection by GCV. This indicates the clones only have the payload as shown in the diagram (bottom, right).

FIG. 3G illustrates an overview of the entire workflow of single integration REWRITE, from payload delivery, Flp-OUT induction, to scarless clone selection.

FIG. 4A depicts flow cytometry data and fluorescence imaging of (top) unmodified mouse embryonic stem cells (mESCs); (middle) landing pad-modified mESCs grown in the presence of blasticidin; and (bottom) landing-pad modified mESCs grown in the absence of blasticidin.

FIG. 4B depicts three derivative landing pad designs with different promoters (eflp; enh; ucoe) driving expression.

FIG. 4C depicts landing pad-modified mESCs from FIG. 4B grown in the presence (left) or absence (right) of blasticidin.

FIG. 5A depicts a diagram for Human Leukocyte Antigen (HLA) locus (left) and class −1 HLA (HLA-1) diversity (right). The 3 megabase HLA locus is the most genetically diverse region in the genome.

FIG. 5B depicts a diagram for HLA-A/B/C deletion and landing pad integration, based on chr6 location. Final output: reusable blank iPSC: HLA-A/B/C−/− with landing pad.

FIG. 5C depicts a diagram of construct design and assembly of 115 kb synHLA (REWRITE compatible). HLA-A has been relocated within the 78-kb intergenic region between HLA-B/C (see FIGS. 6A-6C).

FIG. 6A depicts an illustration of how a synHLA can be integrated.

FIG. 6B depicts an illustration of how HLA expression can be measured by qRT-PCR and immunoblots.

FIG. 6C depicts images of iPSCs, pre- and post-delivery of synHLA (top) and an illustration of human iPSCs (bottom, left), blank accepter human iPSCs with HLA deletion (bottom, middle) and rewritten human iPSCs with synHLA (bottom, right).

FIG. 7 depicts complete removal of HLA-A from human iPSC. Shown on top is PCR verification of complete genomic removal of HLA-A locus from human iPSC. The A PCR amplicon will only amplify if at least one genomic allele of HLA-A has been deleted. The PCR amplicons for internal 1 (In1), internal 2 (In2), and internal 3 (In3) will only amplify if at least one allele of HLA-A is retained. HLA-A(−/−) shows that both alleles of HLA-A locus are removed. HLA-A(−/+) shows that one allele is removed, but that one allele remains intact. The HLA-A locus was removed by a Site-specific nuclease (SSN) in this specific case CRISPR ErCas12a (Mad7).

FIG. 8A depicts an exemplary miniature synHLA construction. (Top left) Order of HLA genes as constructed on mini-synHLA. (Right) Field-Inversion Gel Electrophoresis showing the correct DNA size for the assembled mini-synHLA product. Lane 1—uncut DNA construct. Lane 2—linearized DNA construct. L—NEB midsize DNA ladder. (Bottom left) Integrated Genome Viewer view of the complete sequence of mini-synHLA after sequencing on an Illumina NextSeq.

FIG. 8B depicts the number of clones/colonies obtained after delivering mini-synHLA to a human iPSC containing the REWRITE landing pad and inducible (by doxycycline) iCre. (Top) Dox—doxycycline (no dox −0 dox, 2 days of dox −48 h dox, and 1 day of dox on day 2-24 h day 2 dox) of payload to hiPSC landing pad line. (Bottom) loxFAS or loxF66 containing landing pads with or without co-transfected plasmid with constitutive iCre.

FIG. 9 depicts class-I HLA haplotypes (HLA-A, HLA-B, HLA-C) that would provide immunological matches to various ethnic populations across the world. The 12 listed haplotypes would cover over 50% of northwest Europe and southern China. The table on right shows the percent coverage of various countries across the world.

FIG. 10A depicts the assembly of 115 kb synHLA in yeast. A 10 kB fragment of the HLA-A locus was placed within the 78 kb intergenic region between HLA-B and HLA-C from a BAC clone, and these were assembled into the Payload Vector (PV1f) in yeast.

FIG. 10B depicts a field-inversion gel electrophoresis showing the size of synHLA compared to mini-synHLA.

FIG. 11 depicts a strategy to bi-allelically remove large genomic regions using the REWRITE landing pad and second Deleter construct, expressing Hygromycin resistance and Thymidine Kinase under a PGK promoter. The Deleter construct is flanked by two alternative FRT sites (FRTm) for eventual cassette removal.

FIGS. 12A-12G depict an installation and functional validation of the REWRITE Landing pad in U2OS cells. FIG. 12A shows a simplified schematic drawing of the REWRITE Landing Pad plasmid and minimal REWRITE Payload plasmid. In order to prepare the plasmid for installation at a particular genomic location, location specific homology arms were inserted at the flanking BsaI sites by Golden Gate Assembly. It is important to note that one BsaI site at the 3′ end of the landing pad can be methylated in bacteria. Thus the Landing Pad plasmid must be propagated in Dam/Dcm−/− bacteria prior to adding homology arms. FIGS. 12B-12D shows testing of REWRITE recombination at a REWRITE Landing Pad installed in U2OS cells, the minimal REWRITE Payload was transfected into cells that had been treated with doxycycline and tamoxifen. Cells were observed with a fluorescent microscope nine days post-transfection. A field of cells were imaged in bright-field (FIG. 12B), GFP (FIG. 12C) and RFP (FIG. 12D) channels. FIGS. 12E-G shows groups of cells that have undergone REWRITE recombination outlined in white. Cells that have undergone REWRITE recombination show expression of mNeon Green in the GFP channel (FIG. 12F), but lack mScarlet expression when examined in the RFP channel (FIG. 12G).

FIGS. 13A-13B shows mono-allelic insertion of the REWRITE Landing Pad at the site of HLA-B/C deletion in human iPSCs. FIG. 13A shows coverage map depicting Capture-seq data aligned to the 5′ end of the AHLA-B/C locus with the REWRITE Landing Pad inserted. Coverage in the landing pad region is observed in Clones C12, E5, and E2, at roughly half the level of the surrounding genomic HLA-B/C region indicating mono-allelic insertion of the landing pad. FIG. 13B shows coverage map depicting Capture-seq data aligned to the 3′ end of the AHLA-B/C locus with the REWRITE Landing Pad inserted. Coverage in the landing pad region is observed in all 5 clones, at roughly half the level of the surrounding genomic HLA-B/C region indicating mono-allelic insertion of the landing pad. Based on this data, only Clones C12, E5, and E2 have mono-allelic insertion of a full Re-Write Landing Pad at the HLA-B/C deletion site.

### DETAILED DESCRIPTION

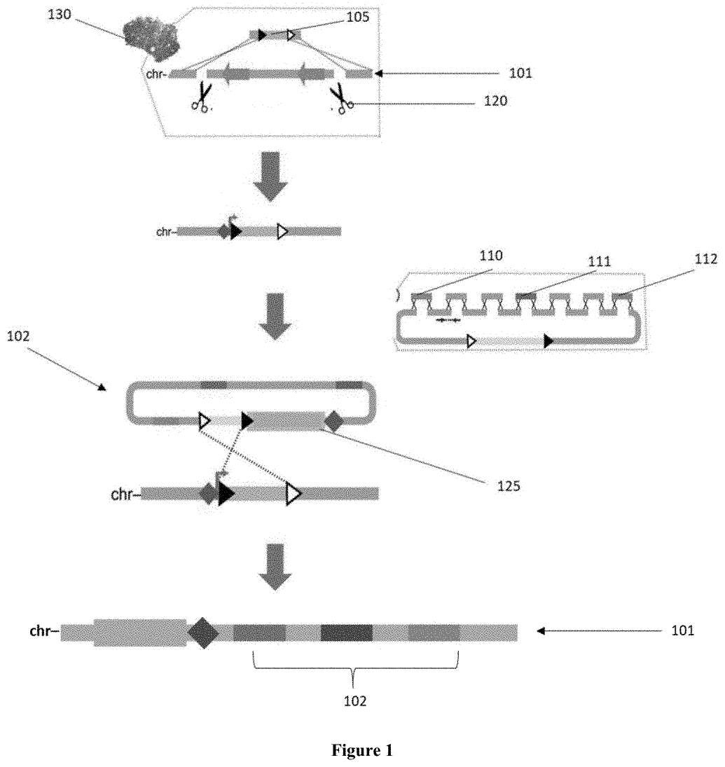

Provided herein are methods and compositions for integrating a DNA construct into a genome, the method comprising contacting the genome in a sample with one or more agents capable of cleaving the genome at a locus, integrating a first nucleic acid sequence into the genome at the locus, and integrating one or more second nucleic acid sequences into the first nucleic acid sequence in the genome, thereby integrating the DNA construct into the genome, wherein each of the one or more second nucleic acid sequences comprises a cargo sequence. In some aspects, provided herein are methods and compositions for integrating a large (sub-megabase) DNA construct, e.g., a DNA construct with at least 10-500 kilobases. In some aspects, methods described herein comprises integrating a landing pad sequence into a genome at a locus and integrating one or more cargo sequences into the landing pad sequence in the genome. In an exemplary workflow, a schematic of megabase-scale genome writing design is shown FIG. 1. In stage 1, a first nucleic acid sequence or a landing pad sequence 105 is integrated into a locus of interest in a genome 101 in a cell derived from a sample 130. This step utilizes one or more agents 120. In stage 2, a second nucleic acid sequence or a DNA construct 102 comprising one or more cargo sequences 110, 111, 112 and a selective marker 125 is prepared and delivered to the cell 130. Finally, the second nucleic acid sequence or the DNA construct 102 is integrated into the first nucleic acid sequence or the landing pad sequence 105 at the locus of interested in the genome 101. This final step can be repeated to integrate multiple DNA constructs sequentially.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well—known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Rewrite

There are limited approaches for site-specifically integrating very large DNA constructs (e.g., >10 kilobases) into human and other mammalian cells, especially primary cells like induced pluripotent stem cells (iPSCs) or embryonic stem cells (ESCs). Furthermore, there are no straightforward and portable (e.g., placed anywhere in the genome including silenced DNA and in any cell type) approaches for integrating large constructs sequentially, to allow for megabase scale DNA integrations. Finally, there are limited approaches for providing “scarless” or “nearly scarless” large DNA integrations, i.e., no remnants of unwanted DNA such as selectable markers like neomycin resistance cassettes. The ability to integrate large DNA constructs into the human genome, especially in human iPSCs, would facilitate the rewriting of the genome, delivery of large genetic circuits, and provide a platform for producing cellular therapies and regenerative medicines. Genome rewriting may provide a tool to create synthetic pathways, synthetic genomes, synthetic cells, or human patient DNA, and a tool for disease modeling.

Recombinase Mediated Cassette Exchange (RMCE) is a general approach for integrating large DNAs into the genome of mouse embryonic stem cells. However, there have been no demonstrations or approaches optimized for integrating large DNA into human stem cells such as iPSCs. RMCE uses tyrosine recombinases (including, but not limited to, Cre or Flp recombinase) and 2 incompatible recombinase recognition sites (including, but not limited to, Cre or Flp recognition sites). For example, a commonly used approach using Cre recombinase uses the incompatible recognition sites loxP and lox2272.

Provided herein is a new RMCE landing pad approach called REcombinase WRiting of Iterative DNA and Trap Excision (REWRITE). In some aspects, REWRITE has optimal features allowing for complete portability to any mammalian cells, including but not limited to, human iPSCs and mouse ESCs; any place in the genome including, but not limited to, silenced regions; allows for sequential integration of large DNA constructs towards the megabase scale; and includes a self-excising selection cassette for rapid and seamless removal of genetic scars, including but not limited to selectable markers. In some aspects, REWRITE landing pad can be placed at any region of the genome. In some embodiments, site-specific nucleases (SSNs) or homology-directed recombination (HR) can be used to place REWRITE landing pad. In some embodiments, SSN comprises meganucleases, zinc-finger nucleases (ZFN), TAL effector nucleases (TALEN), and clustered regularly interspaced short palindromic repeats (CRISPR)/CRISPR-associated (Cas) system.

These four major classes of gene-editing techniques, namely, meganucleases, ZFNs, TALENs, CRISPR/Cas systems share a common mode of action in binding a user-defined sequence of DNA and mediating a double-stranded DNA break (DSB). DSB may then be repaired by HR, an event that introduces the homologous sequence from a donor DNA fragment, or by non-homologous end joining (NHEJ), when there is no donor DNA present.

CRISPR-Cas system may be used with a guide target sequence for genetic screening, targeted transcriptional regulation, targeted knock-in, and targeted genome editing, including base editing, epigenetic editing, and introducing double strand breaks (DSBs) for homologous recombination-mediated insertion of a nucleotide sequence. CRISPR-Cas system comprises an endonuclease protein whose DNA-targeting specificity and cutting activity can be programmed by a short guide RNA or a duplex crRNA/TracrRNA. A CRISPR endonuclease comprises a caspase effector nuclease, typically microbial Cas9 and a short guide RNA (gRNA) or a RNA duplex comprising a 18 to 20 nucleotide targeting sequence that directs the nuclease to a location of interest in the genome. Genome editing can refer to the targeted modification of a DNA sequence, including but not limited to, adding, removing, replacing, or modifying existing DNA sequences, and inducing chromosomal rearrangements or modifying transcription regulation elements (e.g., methylation/demethylation of a promoter sequence of a gene) to alter gene expression. As described above CRISPR-Cas system requires a guide system that can locate Cas protein to the target DNA site in the genome. In some instances, the guide system comprises a crispr RNA (crRNA) with a 17-20 nucleotide sequence that is complementary to a target DNA site and a trans-activating crRNA (tracrRNA) scaffold recognized by the Cas protein (e.g., Cas9). The 17-20 nucleotide sequence complementary to a target DNA site is referred to as a spacer while the 17-20 nucleotide target DNA sequence is referred to a protospacer. While crRNAs and tracrRNAs exist as two separate RNA molecules in nature, single guide RNA (sgRNA or gRNA) can be engineered to combine and fuse crRNA and tracrRNA elements into one single RNA molecule. Thus, in one embodiment, the gRNA comprises two or more RNAs, e.g., crRNA and tracrRNA. In another embodiment, the gRNA comprises a sgRNA comprising a spacer sequence for genomic targeting and a scaffold sequence for Cas protein binding. In some instances, the guide system naturally comprises a sgRNA. For example, Cas12a/Cpf1 utilizes a guide system lacking tracrRNA and comprising only a crRNA containing a spacer sequence and a scaffold for Cas12a/Cpf1 binding. While the spacer sequence can be varied depending on a target site in the genome, the scaffold sequence for Cas protein binding can be identical for all gRNAs.

CRISPR-Cas systems described herein can comprise different CRISPR enzymes. For example, the CRISPR-Cas system can comprise Cas9, Cas12a/Cpf1, Cas12b/C2c1, Cas12c/C2c3, Cas12d/CasY, Cas12e/CasX, Cas12g, Cas12h, or Cas12i. Non-limiting examples of Cas enzymes include, but are not limited to, Cas1, Cas1B, Cas2, Cas3, Cas4, Cas5, Cas5d, Cas5t, Cas5h, Cas5a, Cash, Cas7, Cas8, Cas8a, Cas8b, Cas8c, Cas9 (also known as Csn1 or Csx12), Cas10, Cas10d, Cas12a/|, Cas12b/C2c1, Cas12c/C2c3, Cas12d/CasY, Cas12e/CasX, Cas12f/Cas14/C2c10, Cas12g, Cas12h, Cas12i, Cas12k/C2c5, Cas13a/C2c2, Cas13b, Cas13c, Cas13d, C2c4, C2c8, C2c9, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, GSU0054, Type II Cas effector proteins, Type V Cas effector proteins, Type VI Cas effector proteins, CARF, DinG, homologues thereof, or modified or engineered versions thereof such as dCas9 (endonuclease-dead Cas9) and nCas9 (Cas9 nickase that has inactive DNA cleavage domain). In some cases, the compositions, methods, devices, and systems, described herein, may use the Cas9 nuclease from Streptococcus pyogenes, of which amino acid sequences and structures are well known to those skilled in the art.

In some aspects, described herein, are methods for contacting a genome from a sample with one or more agents capable of cleaving the genome at a locus. In some embodiments, the contacting may occur in vitro. In some embodiments, the contacting may occur in vivo, e.g., in a cell. In some embodiments, the one or more agents comprise a polypeptide, a polynucleotide, or a combination thereof. In some embodiments, the polypeptide comprises an enzyme, e.g., a site-specific nuclease. Examples of a site-specific nuclease are shown above. In some embodiments, a site-specific nuclease comprises an engineered homing endonuclease or meganuclease, a zinc-finger nuclease (ZFN), a transcription activator-like effector nuclease (TALEN), a clustered regularly interspaced short palindromic repeat (CRISPR/Cas), or a combination thereof. In some embodiments, the polynucleotide comprises a guide RNA (gRNA). In some embodiments, the one or more agents comprise a site-specific nuclease and a gRNA (e.g., CRISPR/Cas system).

Agents described herein can be delivered into cells in vitro or in vivo by art-known methods or as described herein. Delivery methods such as physical, chemical, and viral methods are also known in the art [63]. In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus. In some embodiments, agents described herein can be delivered via a carrier. In some embodiments, agents described herein can be delivered by, e.g., vectors (e.g., viral or non-viral vectors), non-vector based methods (e.g., using naked DNA, DNA complexes, lipid nanoparticles, RNA such as mRNA), or a combination thereof. In some embodiments, a carrier can comprise comprises a vector, a messenger RNA (mRNA), double stranded DNA (dsDNA), single stranded DNA (ssDNA), or a plasmid. In some embodiments, agents can be delivered directly to cells as naked DNA or RNA, for instance by means of transfection or electroporation, or can be conjugated to molecules (e.g., N-acetylgalactosamine) promoting uptake by cells.

In some embodiments, vectors can comprise one or more sequences encoding one or more agents described herein. Vectors can also comprise a sequence encoding a signal peptide (e.g., for nuclear localization, nucleolar localization, or mitochondrial localization), associated with (e.g., inserted into or fused to) a sequence coding for a protein. As one example, vectors can include a Cas9 coding sequence that includes one or more nuclear localization sequences (e.g., a nuclear localization sequence from SV40). Vectors described herein can also include any suitable number of regulatory/control elements, e.g., promoters, enhancers, introns, polyadenylation signals, Kozak consensus sequences, or internal ribosome entry sites (IRES). These elements are well known in the art. Vectors described herein may include recombinant viral vectors. Any viral vectors known in the art can be used. Examples of viral vectors include, but are not limited to lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types. In some embodiments, agents described herein may be delivered in one carrier (e.g., one vector). In some embodiments, agents described herein may be delivered in in multiple carriers (e.g., multiple vectors).

In addition, viral particles can be used to deliver agents in nucleic acid and/or peptide form. For example, “empty” viral particles can be assembled to contain any suitable cargo. Viral vectors and viral particles can also be engineered to incorporate targeting ligands to alter target tissue specificity. Non-viral vectors can be also used to deliver agents according to the present disclosure. One example of non-viral nucleic acid vectors is an nanoparticle, which can be organic or inorganic. Nanoparticles are well known in the art. Any suitable nanoparticle design can be used to deliver agents described herein (e.g., nucleic acids encoding such agents).

In some embodiments, agents described herein can be delivered as a ribonucleoprotein (RNP) to cells. An RNP may comprise a nucleic acid binding protein, e.g., Cas9, in a complex with a gRNA targeting a genome/locus/sequence of interest. RNPs can be delivered to cells using known methods in the art, including, but not limited to electroporation, nucleofection, or cationic lipid-mediated methods, for example, as reported by Zuris, J. A. et al., 2015, Nat. Biotechnology, 33(1):73-80.

### Landing Pad Sequence

Provided herein is a method of integrating a DNA construct into a genome, the method comprising contacting the genome from a sample with one or more agents capable of cleaving the genome at a locus, integrating a first nucleic acid sequence into the genome at the locus, and integrating one or more second nucleic acid sequences into the first nucleic acid sequence in the genome, thereby integrating the DNA construct into the genome, wherein each of the one or more second nucleic acid sequences comprises a cargo sequence. In some embodiments, the contacting and/or the integrating may occur in vitro. In some embodiments, the contacting and/or the integrating may occur in vivo, e.g., in a cell. In some embodiments, the first nucleic acid sequence comprises a landing pad sequence. A landing pad sequence as described herein may comprise a gene, a regulatory element, or a combination thereof. Details of a landing pad sequence is described in Example 1. In some embodiments, a regulatory element may comprise a promoter, a terminator, an enhancer, a recombinase recognition site, or a combination thereof. Examples of a promoter can include but are not limited to a universal chromatin opening element (UCOE), human EF1α promoter, Cytomegalovirus (CMV) promoter, simian virus 40 (SV40) promoter, a CAG promoter, or a combination thereof.

In some embodiments, a promoter may be an inducible promoter, a cell specific promoter, a developmental specific promoter, or a tissue specific promoter. Examples of tissue specific promoters include, but are not limited to, a FABP promoter, an Lck promoter, a CamKII promoter, a CD19 promoter, a Keratin promoter, an Albumin promoter, an aP2 promoter, an insulin promoter, an MCK promoter, a MyHC promoter, a WAP promoter, or a Col2A promoter. In some embodiments, the exogenous promoter may be a CMV promoter, an RSV long terminal repeat (LTR) promoter, a simian virus 40 (SV40) promoter, a dihydrofolate reductase (DHFR) promoter, a beta-actin promoter, a phosphoglycerate kinase (PGK) promoter, or an elongation factor-1 alpha (EF1a) promoter. In some embodiments, a promoter may comprise a promoter system comprising one or more promoters, enhancers, or regulatory sequences. For example, a promoter may comprise a cytomegalovirus early enhancer element, a first exon and first intron of chicken beta-actin gene, or a splice acceptor of rabbit beta-globin gene (the CAG promoter system). In some embodiments, a promoter may comprise one or more translational enhancer sequence of human telomerase reverse transcriptase (hTERT), a simian virus 40 (SV40) enhancer sequence, or a CMV enhancer sequence (the SGE promoter system). Promoter systems as described in Watanabe et al. Ocol Rep. 2014 31(3): 1089-1095 is incorporated herein by reference in its entirety. In some embodiments, a promoter may be a CMV promoter. In some embodiments, a promoter can comprise an enhancer region, a TATA box, and a transcription start point. In particular embodiments, a promoter may comprise a CAG promoter system. In another embodiment, a promoter is a SGE promoter system. In some embodiments, a promoter may be constitutive or inducible. In some embodiments, a promoter is a mammalian specific promoter.

Examples of a terminator can include but are not limited to simian virus 40 (SV40) terminator, human growth hormone (hGH) terminator, bovine growth hormone (BGH) terminator, or rabbit beta-globin (rbGlob) terminator. In some embodiments, a landing pad sequence may comprise a gene encoding estrogen receptor 2 (ERT2) or a variant thereof, a gene encoding P2A or a variant thereof, a gene encoding a recombinase or a variant thereof, a blasticidin resistance gene, or a gene encoding a fluorescent protein or a variant thereof. In some embodiments, the ERT2 gene is fused with a gene encoding a recombinase. In some embodiments, the ERT2 gene is fused with a gene encoding a tyrosine recombinase. In some embodiments, the ERT2 gene is fused with a gene encoding a Cre recombinase, a Flp recombinase, a variant thereof, or a combination thereof. In some embodiments, the ERT2 gene is induced by tamoxifen. In some embodiments, a promoter may comprise a human EF1α promoter combined with a universal chromatin opening element (UCOE). In some embodiments, a human EF1α promoter combined with a universal chromatin opening element (UCOE) may serve as a highly active gene promoter.

In some embodiments, a landing pad sequence may comprise one or more restriction enzyme site sequences. Examples of restriction enzyme site sequences can include, but are not limited to, restriction site sequence of restriction enzymes including, but not limited to, AarI, AatII, Acc65I, AccI, AclI, AcuI, AfeI, AflII, AflIII, AgeI, AhdI, AjuI, AleI, AlfI, AloI, AlwNI, ApaI, ApaLI, ApoI, AscI, AseI, AsiSI, AvaI, AvrII, BaeGI, BaeI, BamHI, BanI, BanII, BarI, BbsI, BbvCI, BcgI, BciVI, BcII, BdaI, BfuAI, BglI, BglII, BlpI, Bme1580I, BmeT110I, BmgBI, BmrI, BmtI, Bp1I, BpmI, Bpu10I, BpuEI, BsaAI, BsaBI, BsaHI, BsaI, BsaWI, BsaXI, BseRI, BseYI, BsgI, BsiEI, BsiHKAI, BsiWI, BsmBI, BsmI, BsoBI, Bsp1286I, BspDI, BspEI, BspHI, BspMI, BspQI, BsrBI, BsrDI, BsrFI, BsrGI, BssHII, BssSI, BstAPI, BstBI, BstEII, BstXI, BstYI, BstZ17I, Bsu36I, BtgI, BtgZI, BtsI, ClaI, CspCI, DraI, DraIII, DrdI, EaeI, EagI, Earl, EciI, Eco53kI, Eco57MI, EcoNI, EcoO109I, EcoP15I, EcoRI, EcoRV, Fall, FseI, FspAI, FspI, HaeII, Hin4I, HincII, HindIII, HpaI, KasI, KflI, KpnI, MauBI, MfeI, MluI, MmeI, MreI, MscI, MslI, MspA1I, NaeI, NarI, NcoI, NdeI, NgoMIV, NheI, NmeAIII, NotI, NruI, NsiI, NspI, PacI, PaeR7I, PasI, PciI, PflFI, PflMI, PfoI, PluTI, PmeI, Pm1I, PpiI, PpuMI, PshAI, PsiI, PspFI, PspOMI, PspXI, PstI, PvuI, PvuII, RsrII, SacI, SacII, SalI, SapI, SbfI, ScaI, SexAI, SfcI, SfiI, SfoI, SgrAI, SmaI, SmlI, SnaBI, SpeI, SphI, Srfl, SspI, StuI, StyI, TaqII, TatI, TsoI, TspMI, TstI, Tth111I, XcmI, XhoI, XmaI, XmnI, ZraI. In some embodiments, a landing pad sequence may comprise a BsaI restriction enzyme site sequence.

In some embodiments, a landing pad sequence may comprise a gene encoding a recombinase. In some embodiments, the recombinase may be codon-optimized. In some embodiments, the recombinase may be codon-optimized for mammals. Any codon-optimization methods well known in the art can be used. In some embodiments, the recombinase may lack CpG sites. In some embodiments, the recombinase may be codon-optimized for mammals and lack CpG sites. In some embodiments, recombinase expression may be induced. In some embodiments, an inducible gene expression system may be used. Inducible gene expression systems are well known in the art. Examples of inducible gene expression systems can include, but are not limited to, tetracycline-controlled operator system (e.g., LacR/O-based systems, lacR-VP16 chimeric system, Tet-on/Tet-off system, etc.), cumate-controlled operator system, protein-protein interaction-based chimeric system (e.g., rapamycin system, abscisic acid-regulated interaction system, light-induced protein-protein interaction system, etc.), tamoxifen-controlled recombinase system, and riboswitch-regulatable expression system (e.g., bacteria-derived RNA aptamers linked with hammerhead ribozymes (aptazymes), etc.). In some embodiments, inducible gene expression systems described herein may be combined with RNA interference or CRISPR/Cas9 system. In some embodiments, recombinase expression may be induced by a tetracycline-controlled operator system. In some embodiments, recombinase expression nay be induced by a Tet-on/Tet-off system. In some embodiments, a Tet-on/Tet-off system comprises a promoter. In some embodiments, a Tet-on/Tet-off system comprises a TET-On 3G promoter (TRE3G). In some embodiments, recombinase may be induced by tetracycline or a derivative thereof. In some embodiments, recombinase expression may be induced by doxycycline.

In some embodiments, a landing pad sequence may comprise a gene encoding a fluorescent protein. For example, a landing pad sequence may comprise a gene encoding a red fluorescent protein (RFP), a green fluorescent protein (GFP), a yellow fluorescent protein (YFP), a blue fluorescent protein (BFP), or a cyan fluorescent protein (CFP), or any other fluorescent protein known in the art. In some embodiments, a landing pad sequence may comprise a GFP. In some embodiments, a landing pad sequence may comprise an RFP. In some embodiments, a landing pad sequence may comprise an mNeongreen (GFP).

In some embodiments, a landing pad sequence may comprise a recombinase recognition site. Examples of a recombinase recognition site can include, but are not limited to, a Cre recognition site, or a Flp recognition site. Examples of a recombinase recognition site can include, but are not limited to, loxP, loxM, lox2722, loxFAS, lox5171, frt, a variant thereof, or a combination thereof. In some embodiments, a landing pad sequence described herein may comprise a recombinase recognition site placed downstream of or 3′ 5′ to a promoter. For example, a landing pad sequence described herein may comprise a loxP site placed downstream of or 3′ to an EFα promoter. In some embodiments, an incompatible recombinase recognition site may be used. In some embodiments, an incompatible Cre or Flp recombinase recognition site may be used. In some embodiments, one, two or more incompatible recombinase recognition sites may be used. In some embodiments, an incompatible recombinase recognition site may comprise loxP or lox2272. In some embodiments, incompatible recombinase recognition sites loxP and lox2272 may be used.

In some embodiments, a landing pad sequence described herein may comprise a gene encoding a recombinase. A recombinase may be a site-specific recombinase. A site-specific recombinase may include, but are not limited to a Cre recombinase, a Hin recombinase, a Tre recombinase, or a Flippase (Flp) recombinase. A recombinase may comprise a tyrosine recombinase or a serine recombinase. In some embodiments, a recombinase may be induced. In some embodiments, expression of the recombinase may be induced. Inducible gene expression systems described herein or any inducible gene expression systems known in the art can be used. In some embodiments, the recombinase may be doxycycline inducible. Doxycycline-inducible gene expression systems are well known in the art. In some embodiments, a recombinase is constitutively expressed. In some embodiments, the recombinase may be codon-optimized. In some embodiments, the recombinase may be codon-optimized for mammals. Any codon-optimization methods well known in the art can be used. In some embodiments, the recombinase may lack CpG sites. In some embodiments, the recombinase may be codon-optimized for mammals and lack CpG sites. In some embodiments, the recombinase may comprise Cre recombinase. In some embodiments, the recombinase may comprise codon-optimized Cre recombinase. In some embodiments, the recombinase may comprise a doxycycline inducible codon-optimized Cre recombinase (iCre). In some embodiments, the iCre recombinase is expressed from a plasmid comprising a sequence of SEQ ID NO: 13.

In some embodiments, the recombinase may be self-excisable. In some embodiments, the recombinase may comprise Flp recombinase. In some embodiments, the recombinase may be fused with a tamoxifen inducible estrogen receptor (ERT2). In some embodiments, the Flp recombinase may comprise a self-excising Flp recombinase. In some embodiments, the Flp recombinase may be an inducible Flp recombinase. In some embodiments, the Flp recombinase may be fused with a tamoxifen inducible estrogen receptor (ERT2). In some embodiments, the Flp recombinase may be a self-excising inducible Flp recombinase may be fused with a tamoxifen inducible estrogen receptor (ERT2).

In some embodiments, the landing pad sequence may not comprise a gene encoding a recombinase. In the embodiment where the landing pad sequence does not comprise a gene encoding a recombinase, a recombinase may be transiently expressed from a separate delivery vehicle. A delivery vehicle may be any standard expression vector. In some embodiments, a recombinase may be transiently expressed from a plasmid. In some embodiments, a landing pad sequence may comprise a sequence comprising SEQ ID NO: 2, SEQ ID NO: 5, SEQ ID NO: 6, or SEQ ID NO: 7.

The present disclosure also encompasses a Deleter construct. A Deleter construct can be assembled by standard cloning methods using the same backbone as the REWRITE landing pad. A Deleter construct can comprise a recombination recognition site, a promoter, a marker gene for selection, a sequence that can induce ribosome skipping, a kinase gene, a polyA sequence, or a combination thereof. For example, a Deleter construct can comprise an FRTm (F5) sequence, a PGK promoter (e.g., a constitutive PGK promoter), a hygromycin resistance gene, a P2A sequence, a thymidine kinase gene, an SV40 polyA sequence, and another FRTm (F5) sequence. In some embodiments, a Deleter construct can comprise one or more restriction enzyme digestion sites for cloning. For example, a Deleter construct can comprise BsaI sites for golden gate cloning. In some embodiments, a Deleter construct can comprise homology arms or sequences for genomic integration. In some embodiments, a Deleter construct can comprise a sequence of SEQ ID NO: 15.

### Cargo Sequence

In some aspects, provided herein are methods for integrating multiple DNA constructs into a genome. Also provided herein, in some aspects, are methods for sequentially integrating one or more large DNA constructs. In some aspects, methods described herein may comprise contacting the genome from a sample with one or more agents capable of cleaving the genome at a locus, integrating a first nucleic acid sequence into the genome at the locus, and integrating the one or more second nucleic acid sequences into the first nucleic acid sequence in the genome, thereby integrating the DNA construct into the genome, wherein each of the one or more second nucleic acid sequences comprises a cargo sequence. In some embodiments, the integrating may occur in vitro. In some embodiments, the integrating may occur in vivo, e.g., in a cell. In some embodiments, the one or more second nucleic acid sequences may be integrated sequentially. In some embodiments, the one or more second nucleic acid sequences, each comprising a cargo sequence, may be integrated sequentially. In some embodiments, methods described herein can comprise integrating a first nucleic acid sequence comprising a landing pad sequence and then integrating one or more second nucleic acid sequences each comprising a large DNA “payload” delivery construct or a cargo sequence into the landing pad sequence. In some embodiments, the one or more second nucleic acid sequences may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleic acid sequences.

In some aspects, provided herein, are methods for integrating a large DNA construct. In some embodiments, the integrating may occur in vitro. In some embodiments, the contacting and/or the integrating may occur in vivo, e.g., in a cell. A DNA construct can be inserted into a suitable expression vector or a vector comprising an expression cassette known in the art. A vector can include a transcriptional unit comprising an assembly of a genetic element or elements having a regulatory role in gene expression, for example, promoters or enhancers, a structural or coding sequence which is transcribed into mRNA and translated into protein, and appropriate transcription initiation and termination sequences. In some embodiments, the one or more second nucleic acid sequences are provided by one or more vectors configured to carry a kilobase-sized or megabase-sized (e.g., 10 to 500 kilobases or 10-1000 kilobases) nucleic acid sequence. In some embodiments, vectors described herein can include expression vectors such as mammalian expression vectors. Any suitable expression vector known in the art can be used for delivery. In some embodiments, vectors described herein may comprise a marker. In some embodiments, vectors described herein may comprise a marker that is devoid of a promoter. In some embodiments, vectors described herein may comprise a marker that is devoid of a start codon. In some embodiments, vectors described herein may comprise a marker that is devoid of a promoter and a start codon. In some embodiments, a marker comprises a selectable marker. Examples of a selectable marker can include, but are not limited to an antibiotic marker, a fluorescent marker, or a autotrophic marker. In some embodiment, a marker comprises an antibiotic marker. Examples of an antibiotic marker can include, but are not limited to, neomycin (G418), puromycin, hygromycin, blasticidin, ampicillin, tetracycline, zeocin, kanamycin, bleomycin, chloramphenicol, spectinomycin, streptomycin, carbenicillin, bleomycin, erythromycin, and polymyxin B. Examples of a fluorescent marker can include, but are not limited to, a green fluorescent protein (GFP), a red fluorescent protein (RFP), a yellow fluorescent protein (YFP), a blue fluorescent protein (BFP), or a cyan fluorescent protein (CFP). In some embodiments, a fluorescent marker may comprise a GFP. In some embodiments, a fluorescent marker may comprise a mNeongreen (GFP). In some embodiments, a vector for delivering a DNA construct or a DNA payload may not comprise a promoter. In some embodiments, a vector for delivering a DNA construct or a DNA payload may comprise an antibiotic marker that may not comprise a start codon.

In some aspects, a “promoter trap” system may be used. “Promoter trap” systems are well known in the art and can include a promoter-less and/or start codon (i.e., ATG)-less reporter gene so that reporter gene expression can occur only when the insertion is within a transcriptional unit and/or in a correct direction. In some embodiments, a “promotor trap” system may comprise a marker. In some embodiments, a “promotor trap” system may comprise an antibiotic marker a fluorescent marker, or a autotrophic marker. In some embodiments, a “promotor trap” system may comprise a promoter-less and/or ATG-less antibiotic marker. In some embodiments, a promoter-less and/or ATG-less puromycin resistance marker may be used. In some embodiments, a “promoter trap” system may comprise a GFP. In some embodiments, a “promotor trap” system may comprise a mNeongreen (GFP). In some embodiments, a “promotor trap” system may comprise a promoter/ATG-less puromycin resistance marker and mNeongreen (GFP).

In some embodiments, a vector may be prepared based on a copy-number inducible Bacterial Artificial Chromosome (BAC), Yeast Artificial Chromosome (YAC), or a combination thereof. In some embodiments, a vector may comprise the “promoter trap” described herein. Any suitable vector system described herein can be used to deliver a cargo sequence. In some embodiments, one or more vectors each comprising a cargo sequence can be introduced to a cell or a genome sequentially or serially. In some embodiments, the last vector comprising a cargo sequence may comprise a final “exit” markers or elements. In some embodiments, the final exit markers or elements may include a tamoxifen inducible Flp-ert2 (Flp) and/or a second frt site.

In some embodiments, a cargo sequence (also referred to as a DNA payload, a DNA construct, or “Big-DNA” herein) may comprise at least 10 kilobases. In some embodiments, a cargo sequence may comprise at least 1, 2, 3, 4, 5, 6, 7, 8, 9, 10, 15, 20, 25, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or at least 1000 kilobases. In some embodiments, a cargo sequence may comprise approximately 1, 2, 3, 4, 5, 6, 7, 8, 9, 10, 15, 20, 25, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or approximately 1000 kilobases.

In some embodiments, a cargo sequence may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 120, 140, 150, 160, 180, 200, 22, 240, 250, 260, 280, 300, 350, 400, 450, 500, 550, 600, 650, 700, 750, 800, 850, 900, 950, or at least 1000 megabases. In some embodiments, a cargo sequence may comprise approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 120, 140, 150, 160, 180, 200, 22, 240, 250, 260, 280, 300, 350, 400, 450, 500, 550, 600, 650, 700, 750, 800, 850, 900, 950, or approximately 1000 megabases.

In some embodiments, a cargo sequence may comprise approximately 350 kilobases. In some embodiments, a cargo sequence may comprise approximately 500 kilobases. In some embodiments, one or more cargo sequences may be integrated at each step sequentially into the landing pad within an iPSC or ESC or any other cell type. In some embodiments, a cargo sequence may be cloned into a payload delivery vector.

### Locus

Landing pads, large DNA payloads, cargo sequences, or “Big-DNA” described herein can be integrated into any genomic locus. Genomic loci described herein can include, but are not limited to HLA, AAVS1, CCR5, hROSA26, CYBB, CD40LG, CDK6, TLR8, TRBC1, HPRT, or HOXA. Table 1 describes exemplary genomic loci. In some embodiments, a genomic locus may be a human genomic locus. Because it may be beneficial to replace any gene in a genome with a synthetic version, for example, to provide a disease-linked allele for functional testing in cell culture or an animal model, in principle any gene in a mammalian genome represents a reasonable position to install a landing pad. In some embodiments, a payload vector comprising a location specific homology arm sequences can comprise a sequence of SEQ ID NO: 16.

TABLE 1
Exemplary Human Genomic Locus

| Gene | Locus | Description |
|---|---|---|
| HLA | chr6 | Silenced |
| AAVS1 | chr19 - 19q13.42 | Safe harbor |
| CCR5 | chr3 - p21.31 | HIV1 |
| hROSA26 | chr3 - p26.1 | Safe harbor |
| CYBB | chrX - p11.4 | Active region |
| CD40LG | chrX - q26.3 | Silenced |
| CDK6 | chr7 - 7q21.3 | Active region |
| TLR8 | chrX - p22.2 | Silenced |
| TRBC1 | chr7 - q34 | Silenced |
| HPRT | chrX | Active |
| HOXA | Chr17 | Silenced |

### Biological Samples and Cells

In some embodiments, a sample can be derived from a biological sample, i.e., extracted from a biological sample. In some embodiments, the biological sample can be from a virus, bacterium, mycoplasma, parasite, fungus, or plant. In some embodiments, the biological sample can be an animal. In some embodiments, the animal is a mammal. In some embodiments, the mammal comprises a human, non-human primate, rodent, caprine, bovine, ovine, equine, canine, feline, mouse, rat, rabbit, horse or goat. In some embodiments, a biological sample is obtained from a human subject. The human subject can be a patient. The human subject can be an adult, an adolescent, a pre-adolescent, a child, a toddler, an infant, or a neonate. In some embodiments, the biological sample can be a tissue sample or bodily fluid, such as a human bodily fluid. For example, the bodily fluid can be blood, sera, plasma, lavage, urine, cerebrospinal fluid (CSF), sputum, saliva, bone marrow, synovial fluid, aqueous humor, amniotic fluid, cerumen, breast milk, bronchoalveolar lavage fluid, semen, prostatic fluid, Cowper's fluid, pre-ejaculatory fluid, female ejaculate, sweat, tears, cyst fluid, pleural fluid, peritoneal fluid, pericardial fluid, lymph, chyme, chyle, bile, interstitial fluid, menses, pus, sebum, vaginal secretion, mucosal secretion, stool water, pancreatic juice, lavage fluid from sinus cavities, bronchopulmonary aspirate, blastocoel cavity fluid, or umbilical cord blood. A biological sample can comprise a cell, such as a stem cell, undifferentiated cell, differentiated cell, or a cell from a diseased subject or a subject suspected of having a condition or infection. A biological sample can be blood, a cell, a population of cells, a quantity of tissue, or fluid of a subject. In some embodiments, a biological sample comprises nasopharyngeal fluid, oropharyngeal fluid, saliva, blood, sera, plasma, lavage, urine, ear exudate, cerebrospinal fluid (CSF), sputum, saliva, bone marrow, synovial fluid, aqueous humor, amniotic fluid, cerumen, breast milk, auroral pharyngeal lavage fluid, bronchoalveolar lavage, bronchoalveolar lavage fluid, semen, prostatic fluid, Cowper's fluid, pre-ejaculatory fluid, female ejaculate, sweat, tears, cyst fluid, pleural fluid, peritoneal fluid, pericardial fluid, lymph, chyme, chyle, bile, interstitial fluid, menses, pus, sebum, vaginal secretion, mucosal secretion, stool, stool water, pancreatic juice, lavage fluid from sinus cavities, bronchopulmonary aspirate, blastocoel cavity fluid, or umbilical cord blood.

A biological sample can be collected by any non-invasive means, such as, for example, by a nasopharyngeal swab, a nasal swab, an oropharyngeal swab, or a buccal swab. A biological sample can be also collected by drawing, for example, blood or any other bodily fluid from a subject, or using fine needle aspiration or needle biopsy. A biological sample can be collected by the subject providing the sample to, for example, a doctor or lab technician. For example, the subject can provide a urine, stool, or saliva sample.

A cell described herein can be a bacterial cell, a yeast cell, a fungal cell, an insect cell, or a mammalian cell. In some embodiments, a cell may comprise a mammalian cell. Mammalian cells can be derived or isolated from a tissue of a mammal. In some embodiments, mammalian cells may comprise COS cells, BHK cells, 293 cells, 3T3 cells, NSO hybridoma cells, baby hamster kidney (BHK) cells, PER.C6™ human cells, HEK293 cells or Cricetulus griseus (CHO) cells. In some embodiments, a mammalian cell may comprise a human cell or a mouse cell. Examples of mammalian cells can also include but are not limited to cells from humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some embodiments, a mammalian cell is a human cell. In some embodiments, a mammalian cell is a mouse cell. In some embodiments, a mammalian cell comprises an embryonic stem cell (ESC), a pluripotent stem cell (PSC), or an induced pluripotent stem cell (iPSC).

A cell described herein can be a primary cell or a derivative of a primary cell. Primary cells may be taken directly from a biological sample such as living tissue (e.g., biopsy material) and established for growth in vitro. In some embodiments, primary cells described herein may have undergone very few population doublings. In some embodiments, a primary cell comprises an embryonic stem cell (ESC), a pluripotent stem cell (PSC), or an induced pluripotent stem cell (iPSC). In some embodiments, a primary cell is an embryonic stem cell (ESC). In some embodiments, a primary cell is a pluripotent stem cell (PSC). In some embodiments, a primary cell is an induced pluripotent stem cell (iPSC).

### Diseases and Conditions

Cellular therapies hold great promise for combating previously intractable diseases (e.g., cancer, organ failure, neuropathy, autoimmunity, etc.). Clinical trials are underway for immunotherapies (T-cells), Parkinson's disease (dopaminergic neurons), heart failure (cardiomyocytes), leukemia (cord blood), and organ transplantation [1-10]. For autologous cell therapies, the patient's own cells are extracted, genetically modified ex vivo, and re-infused as a cell-based therapeutic. While autologous (patient-derived) cells generally are optimal, this route remains limited for many applications as this process can be expensive, difficult to scale, and limited by the overall fitness, cell expansion capacity, and potential for engineering of the required cell type. Allogeneic (donor-derived) pluripotent stem cells (PSCs) or embryonic stem cells (ESCs) are a promising alternative as they can be differentiated to many therapeutic cell types and grown indefinitely, providing a potentially unlimited supply of therapeutic cells (e.g., retinal cells for ocular diseases; vascular progenitor cells for ischemia and vascular repair; cardiac progenitors for heart diseases; insulin-producing (3-cells for diabetes; diabetic retinopathy; chronic obstructive pulmonary disease (COPD); organ failure; critical limb ischemia; platelets, red blood cells and CAR-T/Natural Killer cells for cancer, transfusion and hematological disorders). Importantly, PSCs derived from healthy donors (allogeneic) can reduce costs, enable centralized manufacturing, and open new market possibilities and opportunities.

However, allogeneic cells are limited by the need to match Human Leukocyte Antigen (HLA) Class-1 alleles, the most genetically polymorphic region in humans. Mismatches in HLA Class 1 haplotypes lead to the “self vs non-self” immune response that can result in rejection of transplanted therapeutic cells. This process is mediated by genetically polymorphic HLA Class I proteins HLA-A, HLA-B, and HLA-C, (HLA-A/B/C), which are found on the surface of most somatic cells. HLA-A/B/C proteins complex with non-polymorphic beta-2-microglobulin (B2M) and together present intracellular peptide antigens to immune cells. Polymorphisms in HLA-A/B/C alter how these internal peptides are displayed; this gives many patients a unique cellular “fingerprint.” Thus, the immune system rejects allogeneic cells with foreign HLA haplotypes. As the HLAs are the most genetically diverse region in the genome, this leads to difficulty in matching allogeneic donors to patients in need.

This unmet need needs to be addressed by developing a technology to program any HLA haplotype into a PSC on-demand. Pre-immune matched iPSCs can provide an “off-the-shelf” source of cells for regenerative medicine. Provided herein are methods and compositions for “immune-matching” technology that can provide isogenic PSCs that are compatible with any individual, including, but not limited to those with rare HLA haplotypes under-served by PSC “banks.” Such engineered cells can be centrally manufactured into any differentiated cell type, including, but not limited to neurons, muscles, etc. Before differentiation, the PSC can also be modified with additional genetic safeguards and efficacy enhancing features beyond just HLA Class 1 genes, thus reducing time and effort.

Provided herein are methods and compositions for generating a blank human PSC line that is devoid of HLA-1 proteins and capable of accepting a new HLA-1 haplotype. In some embodiments, methods described herein may utilize gene editing technology well known in the art. Also provided herein are methods and compositions for reconfiguring and synthesizing HLA Class 1 as a single linked 115-kb locus, with modular assembly features for inputting any patient haplotype on-demand. Further provided herein are methods and compositions for integrating a 115-kb donor HLA Class 1 locus into a blank stem cell line, while preserving the native regulatory information and expression levels of the HLA Class 1 genes. The technologies described herein can provide personalized stem cells for both immunotherapy and regenerative medicine applications, leading to more efficacious and safer cell-based treatments. The technologies described herein can also provide a cost-effective resource for allogeneic cell therapies. The methods described herein for developing technologies to make “off-the-shelf” stem cells compatible with the immune system of any patient, especially underserved patients with rare immune determinants, as a source to make any adult cell-type suitable for numerous applications in immunotherapy and regenerative medicine.

Methods described herein for genetically engineering immune-matches using a single well-defined PSC, enabling centralized good manufacturing practice (GMP) “off-the-shelf” cells for differentiation to therapeutic cells. This is in contrast to three other approaches, each with major limitations, as described below (Table 1).

(1) Autologous iPSCs. Patient-derived iPSCs obviate the need for HLA matching. However, methods that generate bespoke iPSCs are inefficient and cost-prohibitive [11], largely because each new line must be validated against genetic abnormalities for clinical GMP safety and assessed for differentiation capacity [12,13]. Furthermore, any genetic modifications (e.g., genomic safeguards) would have to be introduced and verified anew each time.

(2) iPSC Banking. Initiatives to provide banks of iPSCs covering common HLA haplotypes are underway in the US, UK, and Japan, but at a cost of $250 million and screening of >160,000 people [14,15]. Further, each banked cell line will require cGMP safety testing as above. Finally, due to genetic and epigenetic differences, each line will vary in ability to differentiate to other cell types, making centralized manufacturing infeasible.

(3) “Immune-Cloaking” Technology. So-called “universal” allogeneic iPSCs can be generated by deleting the B2M gene, thus eliminating HLA Class I cell surface localization [16]. This “hides” cells from recipient T-cells, but instead induces the missing-self response, alerting natural killer cells to destroy HLA-less cells. Many companies attempt to circumvent this issue by expressing HLA-E/G [17], as these HLAs are immune-suppressing. However, this approach has a critical flaw: if these cells are infected by a virus, bacterium, fungus or become cancerous, they'll become invisible reservoirs of disease because the immune system fails to recognize this dangerous cell population as they no longer display any foreign antigens or neoantigens. Indeed, the loss of HLA expression is a very common mechanism by which cancer avoids elimination by the immune system [18,19]. For cell therapies, this issue poses a serious safety concern for long-term transplantation of immune-cloaked cells. And last but not least, many natural killer cells do not express NKG2A (which HLA-E inhibits); this population of natural killer cells is expected to eliminate HLA-E/G expressing cells [16,20,21].

TABLE 2
Comparison of Technologies

| | iPSC | B2M−/− | synHLA |
|---|---|---|---|
| HLA-match | + | | + |
| Off-the-shelf | | + | + |
| Isogenic | | + | + |
| cGMP compatible | | + | + |
| Scalable/low-cost | | + | + |
| NK-cell resistant | + | | + |
| Immune-surveillance | + | | + |

Provided herein are methods and compositions for “immune-matching” strategies that has crucial advantage of preserving essential immune surveillance capabilities as compared to the “immune-cloaking” approach described above. Strategies described herein simultaneously capture the major advantages offered by iPSC banks and immune-cloaking technology (Table 2). Furthermore, immune-matched iPSCs described herein can serve individuals from underrepresented minorities and those with rare HLA haplotypes, a critical unmet need. In addition, the greatest advantage of this off-the-shelf technology is its potential as a standardized platform cell: a uniform, isogenic cell that might be optimized for specific tasks and therapeutics. For example, cells could include ultra-safe features like kill-switches or immune modulating receptors [22-24].

A key innovation is that by reconfiguring HLA-A/B/C as a single linked synthetic locus (“synHLA”), custom HLA haplotypes can be delivered in a single shot directly into a blank acceptor cell (lacking HLA-AB/C). As iPSCs can be differentiated into any therapeutic cell type and can be a preferred cell type, in some embodiments. The inventors' platform technology enables the generation of 100+kb DNA (“Big-DNA”) and its integration into cells using highly specific recombinases or homology-directed repair using site-specific nucleases (CRISPR, TALENs, ZFNs) [25].

Finally, experimental evidence suggests that allogeneic transplants with a homozygous haplotype (e.g., a haplomatch) will be well tolerated [28,29]. This would allow generating a bank of pre-matched cells. A bank of 140 homozygous haplotypes has been estimated to cover 90% of the Japanese population, for example, but would require screening 160,000 individuals [15]; similar numbers are estimated for the UK population [30]. The HLA-matching technology described herein could, in principle, provide haplomatches rapidly and be installed in any cell type that is renewable in culture. It has been computationally determined that just 10 haplotypes can cover 30% of a diverse California population, and can be a reasonable first goal. 50% of people can be covered with just 46 haplotypes. Finally, only matching 2 HLAs may be required for transplants [31-33], and it has been calculated that using this method, a bank of 29 haplotypes could cover 73% of people. HLA haplotype data was taken from the website [34].

In some embodiments, HLA Class II protein deletion may not be necessary for most cell types, because HLA Class II protein are only expressed in professional antigen presenting cells [44,45]. In some embodiment, successful proof-of-principle for HLA-A/B/C could establish feasibility for HLA Class II, which would be useful for fully manipulating dendritic cell antigen presentation.

In some aspects, provided herein, is a method of producing a synthetic genome, the method comprising: a) obtaining a cell from a sample, wherein the cell comprises a genome; b) contacting a first nucleic acid sequence at a first locus on the genome with a first set of one or more agents, wherein the first nucleic acid sequence encodes a first plurality of antigens, and wherein the first locus comprises one or more binding sites for a first set one or more agents; and c) contacting a second nucleic acid sequence at a second locus on the genome with a second set of one or more agents, wherein the second nucleic acid sequence encodes a second antigen, and wherein the second locus comprises one or more binding sites for a second set of one or more agents, thereby producing the synthetic genome.

In some embodiments, the first plurality of antigens and the second antigens comprise a major histocompatibility complex (MHC) or a human leukocyte antigen (HLA). In some embodiments, the MHC or the HLA comprises class I MHC. In some embodiments, the HLA comprises an HLA-A gene, an HLA-B gene, an HLA-C gene, or a combination thereof. In some embodiments, the one or more binding sites comprise a target sequence for the first set or the second set of the one or more agents. In some embodiments, the first set and the second set of the one or more agents comprise a polypeptide, a polynucleotide, or a combination thereof. In some embodiments, the polypeptide comprises an enzyme. In some embodiments, the enzyme comprises a site-specific nuclease. In some embodiments, the site-specific nuclease comprises an engineered homing endonuclease or meganuclease, a zinc-finger nuclease (ZFN), a transcription activator-like effector nuclease (TALEN), a clustered regularly interspaced short palindromic repeat (CRISPR/Cas9), or a combination thereof. In some embodiments, the polynucleotide comprises a guide RNA (gRNA), a single-stranded oligodeoxynucleotide (ssODN), or a combination thereof. In some embodiments, the ssODN comprises at least 50 bp. In some embodiments, the ssODN comprises at least 80 bp. In some embodiments, the ssODN may comprise approximately 50-5000 bp. In some embodiments, the ssODN may comprise at most 5000 bp. In some embodiments, the ssODN may comprise a sequence comprising SEQ ID NO: 3. In some embodiments, the ssODN may comprise a sequence comprising SEQ ID NO: 4. In some embodiments, the first set and the second set of the one or more agents are delivered via a carrier. In some embodiments, the carrier comprises a vector, a messenger RNA (mRNA), double stranded DNA (dsDNA), single stranded DNA (ssDNA), or a plasmid.

In some embodiments, the first locus is at least 1 megabase away from the second locus. In some embodiments, the first nucleic acid sequence and the second nucleic acid sequence comprises at least 3 kilobases. In some embodiments, the first nucleic acid sequence comprises approximately 100 kilobases. In some embodiments, the second nucleic acid sequence comprises approximately 3 kilobases.

In some embodiments, the cell comprises a pluripotent stem cell (PSC) or an induced pluripotent stem cell (iPSC). In some embodiments, the sample comprises a biological sample. Examples of a biological samples is described in “Biological Samples and Cells section”.

In some embodiments, the method further comprises integrating a third nucleic acid sequence at the second locus on the genome after step c). In some embodiments, the integrating comprises homology-directed recombination (HR). In some embodiments, integrating comprises utilizing a recombinase. In some embodiments, the recombinase is constitutively expressed. In some embodiments, the recombinase is inducible, i.e., the expression of the recombinase is inducible. In some embodiments, the recombinase is a site-specific recombinase. In some embodiments, the recombinase comprises a Cre recombinase, a Flp recombinase, or a combination thereof. In some embodiments, integrating comprises utilizing an inducible or constitutive site-specific recombinase Cre, a Flp recombinase, or a combination thereof. In some embodiments, integrating comprises utilizing an inducible or constitutive site-specific recombinase Cre, an inducible or constitutive site-specific Flp recombinase, or a combination thereof. In some embodiments, the third nucleic acid sequence is linear. In some embodiments, the third nucleic acid sequence comprises a sequence homologous to the second locus on the genome. In some embodiments, the third nucleic acid sequence comprises a HLA-A gene, a HLA-B gene, a HLA-C gene, or a combination thereof. In some embodiments, the third nucleic acid sequence further comprises a regulatory element sequence. In some embodiments, the regulatory element sequence comprises a promoter sequence, an enhancer sequence, a terminator sequence, or a combination thereof. In some embodiments, the third nucleic acid sequence further comprises an intergenic sequence. In some embodiments, the intergenic sequence comprises at least about 50 kilobases. In some embodiments, the third nucleic acid sequence further comprises one or more loxP/loxM sites. In some embodiments, the third nucleic acid sequence comprises at least 20 kilobases. In some embodiments, the third nucleic acid sequence comprises at least 30 kilobases. In some embodiments, the third nucleic acid sequence comprises at least 100 kilobases. In some embodiments, a synHLA may comprise a sequence comprising SEQ ID NO: 1. In some embodiments, a synHLA may comprise a sequence comprising SEQ ID NO: 8. In some embodiments, a synHLA may comprise a sequence comprising SEQ ID NO: 14.

### OTHER EMBODIMENTS

In some aspects, provided herein is a method of integrating a DNA construct into a genome, the method comprising: a) contacting the genome from a sample with one or more agents, wherein the one or more agents are capable of cleaving the genome at a locus; b) integrating a first nucleic acid sequence into the genome at the locus; and c) integrating the one or more second nucleic acid sequences into the first nucleic acid sequence in the genome, thereby integrating the DNA construct into the genomes, wherein each of the one or more second nucleic acid sequences comprises a cargo sequence.

In some embodiments, the first nucleic acid sequence comprises a landing pad sequence. In some embodiments, the landing pad sequence comprises one or more genes, regulatory elements, or combinations thereof. In some embodiments, the one or more regulatory elements comprise a promoter, a terminator, a recombinase recognition site, or a combination thereof. In some embodiments, the promoter comprises a universal chromatin opening element (UCOE), human EF1α promoter, phosphoglycerate kinase (PGK), or a combination thereof. In some embodiments, the terminator comprises simian virus 40 (SV40) terminator, bovine growth hormone (bGH) terminator, human growth hormone (hGH) terminator, or rabbit beta-globin (rbGlob).

In some embodiments, the one or more genes comprise a gene encoding estrogen receptor 2 (ERT2) or a variant thereof, a gene encoding P2A or a variant thereof, a Thymidine Kinase or a variant thereof, a gene encoding a recombinase or a variant thereof, a blasticidin resistance gene, a hygromycin resistance gene, or a gene encoding a fluorescent protein or a variant thereof. In some embodiments, the ERT2 gene is fused with a gene encoding a recombinase. In some embodiments, the ERT2 gene is induced by tamoxifen. In some embodiments, the recombinase comprises a tyrosine recombinase. In some embodiments, the recombinase comprises a Cre recombinase, a Flp recombinase, a variant thereof, or a combination thereof. In some embodiments, the recombinase is codon-optimized for mammals. In some embodiments, the recombinase lacks CpG sites. In some embodiments, the gene encoding the recombinase is induced by a gene expression system. In some embodiments, the gene encoding the recombinase is induced by doxycycline.

In some embodiments, the fluorescent protein is a red fluorescent protein, a green fluorescent protein, a yellow fluorescent protein, a blue fluorescent protein or a cyan fluorescent protein. In some embodiments, integrating in steps b) and c) comprises sequential integration. In some embodiments, the recombinase recognition site comprises a Cre recognition site, a Flp recognition site, or a combination thereof. In some embodiments, the recombinase recognition site comprises one or more loxP, loxM, lox2722, loxFAS, lox5171, frt, a variant thereof, or a combination thereof.

In some embodiments, the one or more second nucleic acid sequences are provided by one or more vectors configured to carry nucleic acid sequence that comprises approximately from 10 kilobases to 1,000 kilobases. In some embodiments, the one or more vectors comprise a marker, wherein the marker is devoid of a promoter and a start codon. In some embodiments, the marker comprises a selectable marker. In some embodiments, the selectable marker comprises an antibiotic marker.

In some embodiments, the genome in the sample is contained in a cell. In some embodiments, the contacting and integrating are performed in the cell. In some embodiments, the cell comprises a mammalian cell. In some embodiments, the mammalian cell comprises a human cell or a mouse cell. In some embodiments, the cell comprises a primary cell. In some embodiments, the primary cell comprises an embryonic stem cell (ESC), a pluripotent stem cell (PSC), or an induced pluripotent stem cell (iPSC).

In some embodiments, the one or more agents comprise a polypeptide, a polynucleotide, or a combination thereof. In some embodiments, the polypeptide comprises a site-specific nuclease. In some embodiments, the site-specific nuclease comprises an engineered homing endonuclease or meganuclease, a zinc-finger nuclease (ZFN), a transcription activator-like effector nuclease (TALEN), a clustered regularly interspaced short palindromic repeat (CRISPR), or a combination thereof. In some embodiments, the polynucleotide comprises a guide RNA (gRNA).

In some embodiments, the one or more agents are delivered via a carrier. In some embodiments, the carrier comprises a vector, a messenger RNA (mRNA), a double stranded DNA (dsDNA), a single stranded DNA (ssDNA), or a plasmid. In some embodiments, each of the one or more second nucleic acid sequences comprises at least 10 kilobases, 100 kilobases, or 1 megabase. In some embodiments, each of the one or more second nucleic acid sequences comprise approximately from 1 kilobase to 10 megabases.

In some embodiments, the sample comprises a biological sample. In some embodiments, the biological sample comprises a cell, tissue sample, or blood sample. In some embodiments, the biological sample is obtained from a subject. In some embodiments, the subject is a human.

In some embodiments, the integrating in steps b) and c) comprises homology-directed recombination (HR). In some embodiments, the integrating comprises utilizing an inducible or constitutive site-specific recombinase Cre, a Flp recombinase, or a combination thereof. In some embodiments, the cargo sequence comprises a sequence encoding one or more antigens. In some embodiments, the integrating in steps b) or c) comprise a self-excising recombinase. In some embodiments, the cargo sequence comprises 100 kilobases, 500 kilobases, or 1 megabase.

In some embodiments, the first plurality of antigens and the second antigens comprise a major histocompatibility complex (MHC) or a human leukocyte antigen (HLA). In some embodiments, the MHC or the HLA comprises class I MHC. In some embodiments, the HLA comprises an HLA-A gene, an HLA-B gene, an HLA-C gene, or a combination thereof. In some embodiments, wherein the one or more binding sites comprise a target sequence for the first set or the second set of the one or more agents.

In some embodiments, the first set and the second set of the one or more agents comprise a polypeptide, a polynucleotide, or a combination thereof. In some embodiments, the polypeptide comprises a site-specific nuclease. In some embodiments, the site-specific nuclease comprises an engineered homing endonuclease or meganuclease, a zinc-finger nuclease (ZFN), a transcription activator-like effector nuclease (TALEN), a clustered regularly interspaced short palindromic repeat (CRISPR/Cas), or a combination thereof.

In some embodiments, the polynucleotide comprises a guide RNA (gRNA), a single-stranded oligodeoxynucleotide (ssODN), or a combination thereof. In some embodiments, the ssODN comprises at least 50 bp or at least 80 bp. In some embodiments, the first set and the second set of the one or more agents are delivered via a carrier. In some embodiments, the carrier comprises a vector, a messenger RNA (mRNA), double stranded DNA (dsDNA), single stranded DNA (ssDNA), or a plasmid.

In some embodiments, the first locus is at least 10 kilobases or at least 1 megabase away from the second locus. In some embodiments, the first locus and the second locus are on different chromosomes. In some embodiments, the first nucleic acid sequence and the second nucleic acid sequence comprises at least 3 kilobases. In some embodiments, the first nucleic acid sequence comprises approximately 100 kilobases. In some embodiments, the second nucleic acid sequence comprises approximately 3 kilobases.

In some embodiments, the cell comprises a pluripotent stem cell (PSC) or an induced pluripotent stem cell (iPSC). In some embodiments, the sample comprises a biological sample. In some embodiments, the biological sample comprises a cell, tissue sample, or blood sample.

In some embodiments, the method further comprises integrating a third nucleic acid sequence at the second locus on the genome after step c). In some embodiments, the integrating comprises homology-directed recombination (HR). In some embodiments, the integrating comprises utilizing an inducible or constitutive site-specific recombinase Cre, a Flp recombinase, or a combination thereof.

In some embodiments, the third nucleic acid sequence is linear. In some embodiments, the third nucleic acid sequence comprises a sequence homologous to the second locus on the genome. In some embodiments, the third nucleic acid sequence comprises a HLA-A gene, a HLA-B gene, a HLA-C gene, or a combination thereof. In some embodiments, the third nucleic acid sequence further comprises a regulatory element sequence. In some embodiments, the regulatory element sequence comprises a promoter sequence, an enhancer sequence, a terminator sequence, or a combination thereof.

In some embodiments, the third nucleic acid sequence further comprises an intergenic sequence. In some embodiments, the intergenic sequence comprises at least about 50 kilobases. In some embodiments, the third nucleic acid sequence further comprises one or more loxP/loxM sites. In some embodiments, the third nucleic acid sequence comprises at least 10 kilobases. In some embodiments, the third nucleic acid sequence comprises at least 30 kilobases. In some embodiments, the third nucleic acid sequence comprises at least 100 kilobases.

In some aspect, provided herein, is a method of producing a synthetic genome, the method comprising: a) obtaining a cell from a sample, wherein the cell comprises a genome; b) contacting a nucleic acid sequence encoding an antigen at a locus on the genome with a set of one or more agents, wherein the locus comprises one or more binding sites for the set of the one or more agents; c) integrating a synthetic sequence to the locus on the genome, wherein the integrating the synthetic sequence to the locus on the genome preserves regulatory components for gene expression, thereby producing the synthetic genome.

In some embodiments, the antigen comprises a major histocompatibility complex (MHC) or a human leukocyte antigen (HLA). In some embodiments, the MHC or the HLA comprises class I MHC. In some embodiments, the HLA comprises an HLA-A gene, an HLA-B gene, an HLA-C gene, or a combination thereof. In some embodiments, the one or more binding sites comprise a target sequence for the set of the one or more agents. In some embodiments, the set of the one or more agents comprises a polypeptide, a polynucleotide, or a combination thereof. In some embodiments, the polypeptide comprises a site-specific nuclease. In some embodiments, the site-specific nuclease comprises an engineered homing endonuclease or meganuclease, a zinc-finger nuclease (ZFN), a transcription activator-like effector nuclease (TALEN), a clustered regularly interspaced short palindromic repeat (CRISPR/Cas), or a combination thereof.

In some embodiments, the polynucleotide comprises a guide RNA (gRNA), a single-stranded oligodeoxynucleotide (ssODN), or a combination thereof. In some embodiments, the ssODN comprises at least 50 bp or at least 80 bp. In some embodiments, the set of the one or more agents are delivered via a carrier. In some embodiments, the carrier comprises the carrier comprises a vector, a messenger RNA (mRNA), double stranded DNA (dsDNA), single stranded DNA (ssDNA), or a plasmid.

In some embodiments, the nucleic acid sequence comprises at least 1 kilobases. In some embodiments, the nucleic acid sequence comprises approximately 100 kilobases. In some embodiments, the nucleic acid sequence comprises approximately 3 kilobases. In some embodiments, the cell comprises a pluripotent stem cell (PSC) or an induced pluripotent stem cell (iPSC).

In some embodiments, the sample comprises a biological sample. In some embodiments, the biological sample comprises a cell, tissue sample, or blood sample. In some embodiments, the integrating comprises homology-directed recombination (HR). In some embodiments, the integrating comprises utilizing an inducible site-specific recombinase Cre, a Flp recombinase, or a combination thereof. In some embodiments, the synthetic sequence is linear. In some embodiments, the synthetic sequence comprises a sequence homologous to the locus on the genome. In some embodiments, the synthetic sequence comprises a HLA-A gene, a HLA-B gene, a HLA-C gene, or a combination thereof.

In some embodiments, the synthetic sequence further comprises a regulatory element sequence. In some embodiments, the regulatory element sequence comprises a promoter sequence, an enhancer sequence, a terminator sequence, or a combination thereof. In some embodiments, the synthetic sequence further comprises an intergenic sequence. In some embodiments, the intergenic sequence comprises at least about 50 kilobases. In some embodiments, the synthetic sequence further comprises one or more loxP/loxM sites. In some embodiments, the synthetic sequence comprises at least 10 kilobases. In some embodiments, the synthetic sequence comprises at least 30 kilobases. In some embodiments, the synthetic sequence comprises at least 100 kilobases.

### EXAMPLES

These examples are provided for illustrative purposes only and not to limit the scope of the claims provided herein.

### Example 1: Design and Testing of REWRITE Landing Pad in Mouse Embryonic Stem Cells (mESCs)

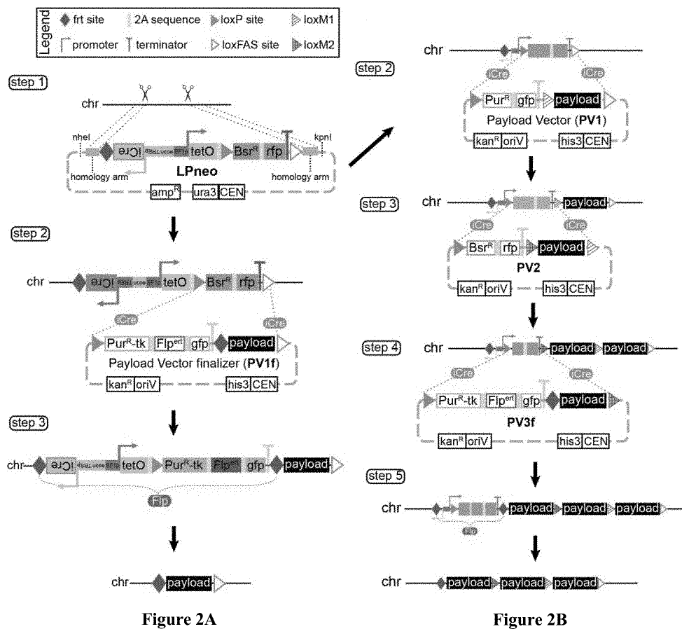

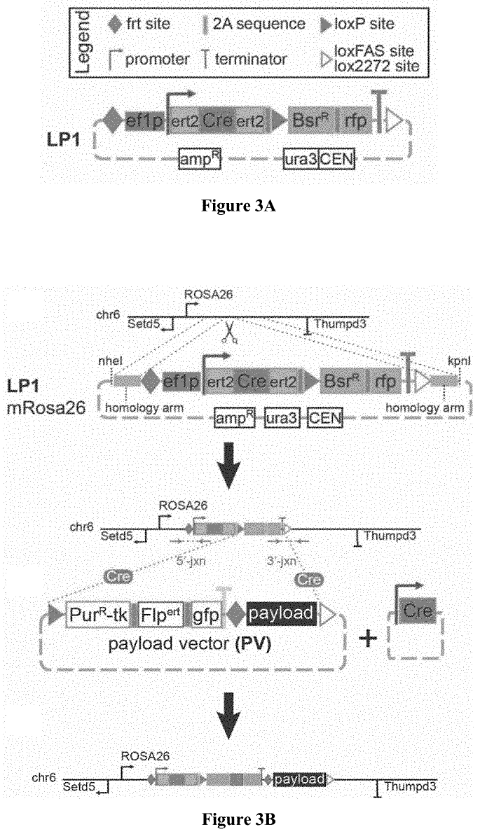

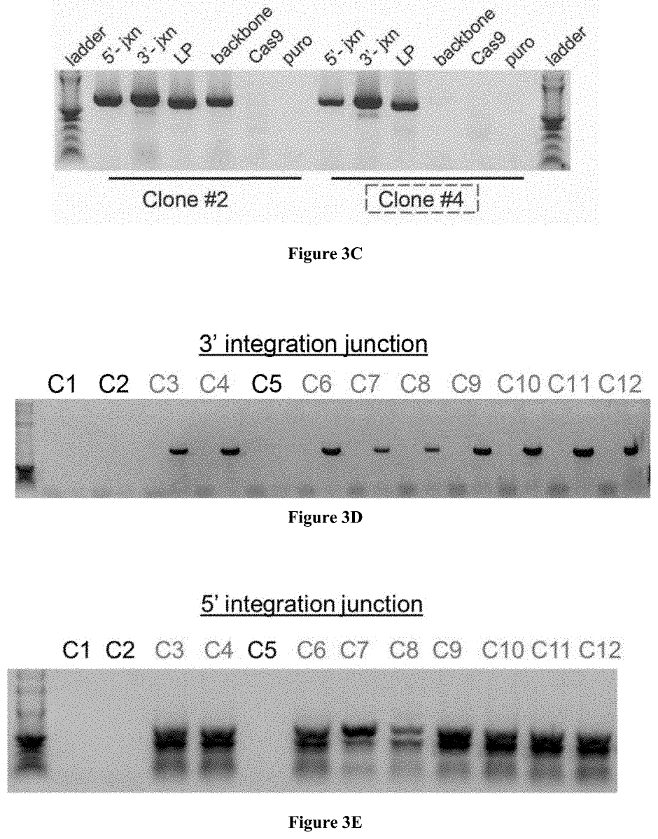

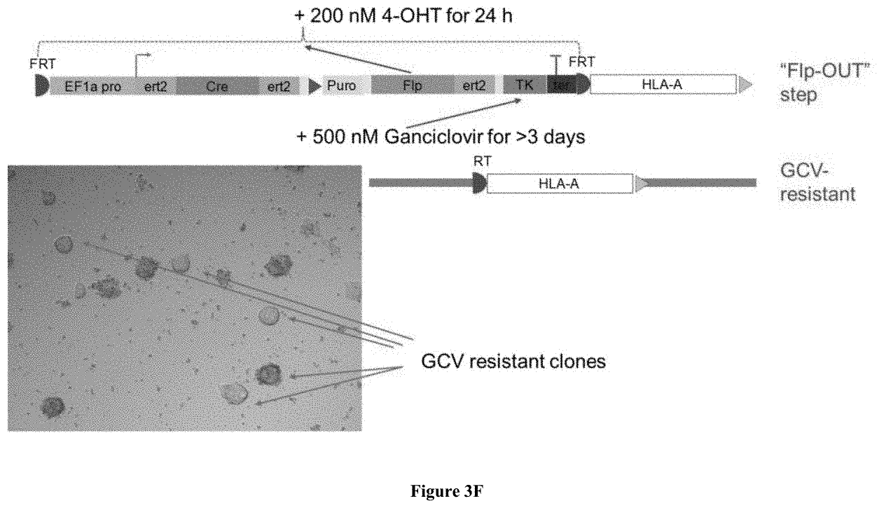

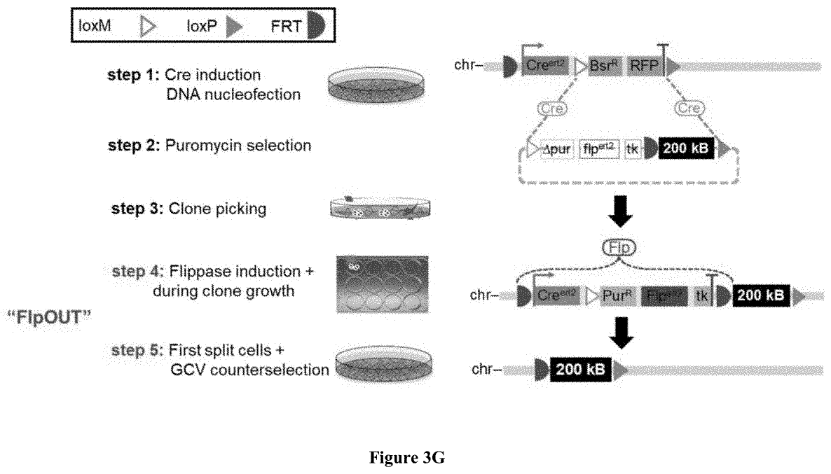

A Recombinase Mediated Cassette Exchange (RMCE) landing pad (LP) approach was designed to write large DNA into the genome of human iPSCs, which, when repeated consecutively, enables megabase genome engineering (FIG. 3G). The ideal landing pad would: permit 100-300+kb integrations consecutively; contain all necessary components; be placed anywhere in the genome; and not leave behind transgene markers (“scarless” delivery). By using CRISPR/Cas9 guide RNAs (gRNA) for homology-directed repair, the landing pad may be site-specifically placed over any amount of DNA (FIG. 2A), thereby enabling the reintroduction of “designer” DNAs of any size. The approach precisely delivers large DNA payloads by using an in-frame “promoter trap,” thereby limiting non-specific genome integrations. The approach moves unidirectionally as new integrants are brought in (FIG. 2A). Finally, the promoter trap and transgenes would be “self-excisable” during early clonal expansion, enabling rapid scarless genome engineering.

The initial LP design (FIGS. 3A-3G) was tested at the murine Rosa26 locus in mouse embryonic stem cells (mESCs), as mESCs are more amenable to genetic modification. The 5.7 kb LP1, in order, (FIG. 3A) consists of: (i) one Flp recognition sequence frt; (ii) constitutively expressed human EF1α promoter; (iii) tamoxifen inducible double ert2 (estrogen receptor) fused Cre recombinase; (iv) ribosome skipping P2A; (v) Cre recognition sequence loxP; (vi) a blasticidin resistance marker (vii) ribosome skipping T2A; (viii) mScarlet (rfp); (ix) SV40 terminator; and (x) a Cre-lox mutant site. Two landing pads were built to test if the pairing of loxP/loxFAS enables higher integration frequencies in human cells than the loxP/lox2272 pairing as previously reported [58]. Both LPs were integrated at Rosa26 using homology-directed repair, using homology arms of −450 bp, and assisted by a single Rosa26 Cas9 gRNA. For each LP, 10-20 clones that were blasticidin resistant and mScarlet positive were identified. Correct genomic integration was assessed through PCR genotyping, using primers that amplify the novel integration junctions (FIGS. 3C-3E). The process of inducing Flp recombinase after payload delivery to provide scarless insertions (“Flp-OUT”) is illustrated in FIG. 3F. Induction of Flp recombinase using 4-hydroxytamoxifin (4-OHT) from within the payload excises the gene cassettes via FRT recombinase-mediated excision. Because the cassettes express Thymidine Kinase, only full excision is resistant to Ganciclovir (GCV), whereas those not excising are not resistant to GCV. GCV resistant stem cell clones are shown in FIG. 3F after payload delivery (as described in FIGS. 3B-3E), induction of 4-OHT, and selection by GCV. These clones only have the payload as shown in the diagram in FIG. 3F, bottom right. An overview of the entire workflow or REWRITE, from payload delivery, Flp-OUT induction, to scarless clone selection is illustrated in FIG. 3G.

Large DNA payloads (100 kb+) are integrated using a Payload Vector (PV) (FIGS. 2B and 3A-3E). The Payload Vector used herein is based on a copy-number inducible Bacterial Artificial Chromosome (BAC) [60] and Yeast Artificial Chromosome (YAC), enabling megabase-sized DNA carrying capacity. The “promoter trap” contains a promoter/ATG-less puromycin resistance marker and mNeongreen (GFP), that only functions by capturing the EF1α promoter by precisely integrating in-frame over the landing pad via Cre RMCE (FIG. 3B, middle). Payloads are brought in serially, and the final “exit” PV includes a tamoxifen inducible Flp-ert2 (Flp) and a second frt site. This enables near simultaneous excision of the markers, thereby leaving a scarless (except one each of lox and frt sites) rewritten locus (FIGS. 2A-2B and 3B, middle). In some embodiments, a payload vector may comprise a sequence comprising SEQ ID NO: 9 or SEQ ID NO: 10. In some embodiments, a payload vector may comprise a sequence comprising SEQ ID NO: 11 or SEQ ID NO: 12.

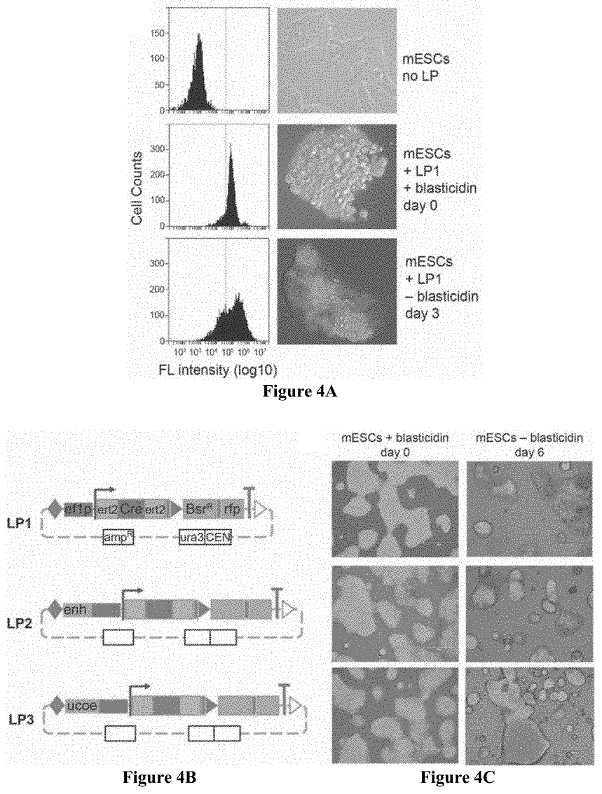

It was found that the tamoxifen inducible double ert2 Cre fusion in LP1 (FIG. 3A) was not active. The attempt to integrate a 10 kb human genome segment at Rosa26 in mESCs produced zero puromycin-resistant cells. It was thought that double ert2 (a configuration meant to reduce background Cre activity) leads to low Cre activity in mammalian cells. In order to test the promoter trap itself, the experiment was re-performed by co-transfecting a plasmid constitutively expressing Cre. In addition, a version of Cre that has been mammalian codon-optimized and lacks CpG methylation sites to prevent gene silencing, called “iCre” was also tested [61]. Both loxFAS and lox2272 containing LP1s were tested. It was found that loxFAS out-performs lox2272 for wt Cre-mediated RMCE, as 33 puromycin-resistant and mNeongreen+clones for loxP/loxFAS were found, but only 2 clones were found for loxP/lox2272. These were also now negative for blasticidin and mScarlet. Furthermore, recoded iCre substantially out-performed wt Cre, as over 270 clones in the loxFAS LP1 were found (FIGS. 4A-4C). In conclusion, iCre with the loxP/loxFAS pairing was found to provide optimal conditions for large DNA genome integrations.

It was also found that LP1 was rapidly silenced without continued use of blasticidin antibiotic in the media, as mScarlet expression diminished within 3 days (FIG. 4A), despite Rosa26 being a “safe-harbor”site. As the goal is for the LP to be placed at any genomic site, the promoter was redesigned to prevent gene silencing. To minimize the silencing effect, two new landing pads (LP2 and LP3) were built to add either the enhancer regions of the EF1α promoter, or a Universal Chromatin Opening Element (UCOE) upstream of the EF1α promoter, previously shown to limit silencing with EF1α [66]. LP2 and LP3 were integrated into Rosa26 in mESCs as before and new stable clones were obtained. To determine whether these modifications reduced silencing, LP1-3 were initially grown with blasticidin selection, and then without blasticidin selection for an additional 6 days (FIG. 4C). Whereas mESC clones with LP1 and LP2 both showed silencing of mScarlet, LP3 containing the UCOE remained fluorescent after 6 days without blasticidin selection (FIG. 4C).

Based on these results, the landing pad was reconstructed to a final configuration called LPneo, which represents the most common REWRITE landing pad configuration (FIG. 2B). To provide optimal Cre activity, the LP was engineered to contain iCre (with SV40 nuclear localization signals) driven by the low background 3rd gen TET-On 3G system (TRE3G; Tet-On® 3G Bidirectional inducible Expression System (with ZsGreen1)). The TRE3G promoter for iCre was cloned as a bidirectional promoter in reverse to the EF1α promoter. The TRE3G activator protein TET-ON, which is inactive without doxycycline, was constitutively expressed from the EF1α promoter. The promoter trap was otherwise unmodified. Type IIS restriction sites BsaI were also introduced to enable Golden Gate assembly of homology arms for rapidly redeploying the LP to new sites. Thus, the final landing pad was constructed to be an “all-in-one” construct with the potential to be used anywhere in the genome.

### Example 2: Integration in Human Induced Pluripotent Stem Cells (iPSCs)

To show that REcombinase WRiting of Iterative DNA and Trap Excision (REWRITE) enables Big-DNA delivery into human induced pluripotent stem cells (hiPSCs), a REWRITE landing pad was generated to remove a 100 kilobase genomic region covering the HLA-B and HLA-C genes on chromosome 6 in iPSCs. Two CRISPR gRNAs for erCas12a, also called MAD7, were designed to generate double strand breaks spanning a 100 kilobase region. The REWRITE landing pad was successfully integrated over this 100 kilobase genomic locus, as the iPSCs were both Blasticidin resistant and mScarlet+.

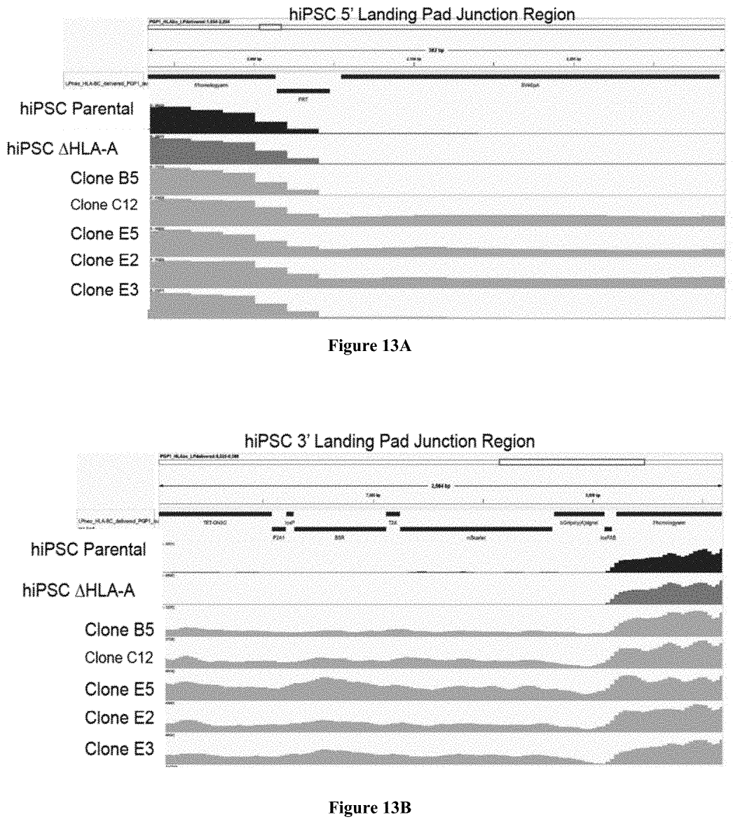

Using the methods described herein, REWRITE landing pad was inserted at the site of HLA-B/C deletion in human iPSCs (FIGS. 13A-13B). AHLA-B/C locus with mono-allelic REWRITE Landing Pad insertion was sequenced using next-generation sequencing platform method, Capture-seq. Capture-seq data aligned to the 5′ end of the AHLA-B/C locus with the REWRITE Landing Pad insertion shows that Clones C12, E5, and E2 have coverage in the landing pad region at roughly half the level of the surrounding genomic HLA-B/C region, which indicates mono-allelic insertion of the REWRITE landing pad (FIG. 13A). Capture-seq data aligned to the 3′ end of the AHLA-B/C locus with the REWRITE Landing Pad insertion shows that Clones B5, C12, E5, E2, and E3 have coverage in the landing pad region at roughly half the level of the surrounding genomic HLA-B/C region, which indicates mono-allelic insertion of the REWRITE landing pad (FIG. 13B). Based on this data, Clones C12, E5, and E2 were confirmed to have mono-allelic insertion of a full REWRITE Landing Pad at the HLA-B/C deletion site.

A 115 kilobase synthetic version of the human HLA locus, called synHLA, was assembled into our Payload Vector (PV). The 115 kilobase PV was introduced into the REWRITE iPSCs via nucleofection, and the iCre expression module was then activated for 3 days using Doxycycline. After 3 days, successful integrants was selected using Puromycin. After 10 days, −30 distinct colonies indicative of Big-DNA delivery were observed. Under fluorescent microscopy it was confirmed that all clones were mNeongreen+in addition to Puromycin resistant. This confirms that the REWRITE landing pad approach enables the delivery of Big-DNA of at least 115 kilobases.

### Example 3: Engineering a Blank (HLA-Less) iPSC by Replacing HLA-A/B/C with a Landing Pad

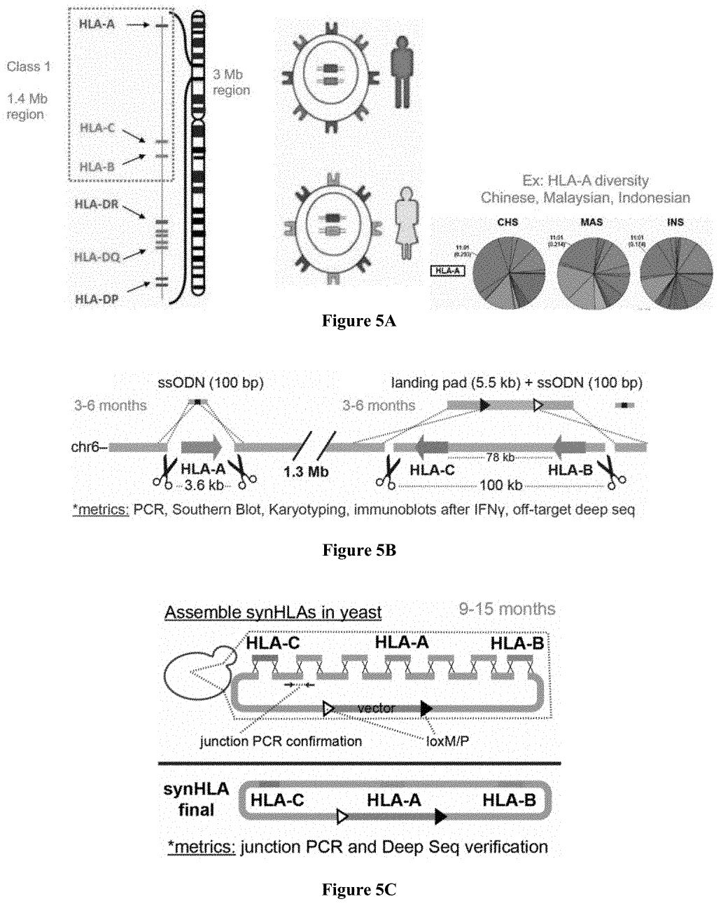

A well-characterized iPSC (PGP1) from the Coriell Intitute was acquired [50] for this experiment; however, any human iPSC can be used. Starting with this iPSC line, a “blank” iPSC devoid of HLA-A/B/C and encoding the Recombinase Mediated Cassette Exchange (RMCE) landing pad will be constructed (FIG. 5B). The blank iPSC allows testing precise single-copy integration of synHLAs. Following HLA deletion, the blank iPSC will be evaluated for potential off-target mutations by deep sequencing and karyotype analysis. No issues associated with bi-allelic deletion of HLA-1 genes in human iPSCs are expected because HLA-less mouse ESCs readily generate healthy mice [51].

The 100 kb HLA-B/C locus will be replaced with the RMCE landing pad (5.5 kb) described earlier by CRISPR or TALEN or ZFN (heretofore “Site-Specific Nucleases” or “SSNs”) DNA-cut stimulated homology-directed recombination (HR). The other allele will be cleanly deleted with a commercially synthesized 100 bp single stranded oligodeoxynucleotides (ssODN) deletion fragment [46,47]. Two SSN guide RNAs will be introduced, one upstream of HLA-C and one downstream of HLA-B. The landing pad construct will be introduced as linear dsDNA designed with homology arms to the region (FIG. 5B). The Site-Specific Nuclease will be delivered transiently on plasmids. The HLA-B/C deletion genotype will be confirmed by PCR, Southern Blotting, immunoblots after IFN-γ HLA induction [17] (PSCs don't express HLA), karyotyping, and potential predicted off-target mutations will be evaluated by deep sequencing.

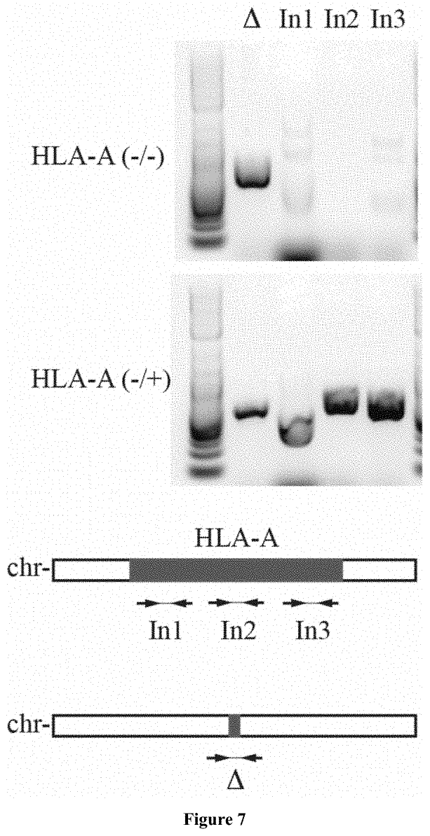

The final step in preparing the blank iPSC requires deleting HLA-A (3.6 kb) (FIG. 5B), as it is located 1.3 Mb away from HLA-B/C on chromosome [6]. The two HLA-A alleles will be deleted using two flanking SSNs and a commercially synthesized 100 bp ssODN [46,47]. The clones will be verified for HLA-A deletion as detailed in the above for HLA-B/C deletion. HLA-A allele removal from human iPSC using an SSN, CRISPR ErCas12a (Mad7) is shown in FIG. 7.

Alternative Methods:
1. Instead of a iPSCs, a hESC (H1) cell line with another landing pad inserted in X-chromosome.
2. A single MAD7 targeted to an exon for mutagenic non-homologous end-joining (NHEJ) can be used for HLA-B/C deletion.
3. Deletions can be repeated using multiple alternative MAD7 gRNAs in parallel.

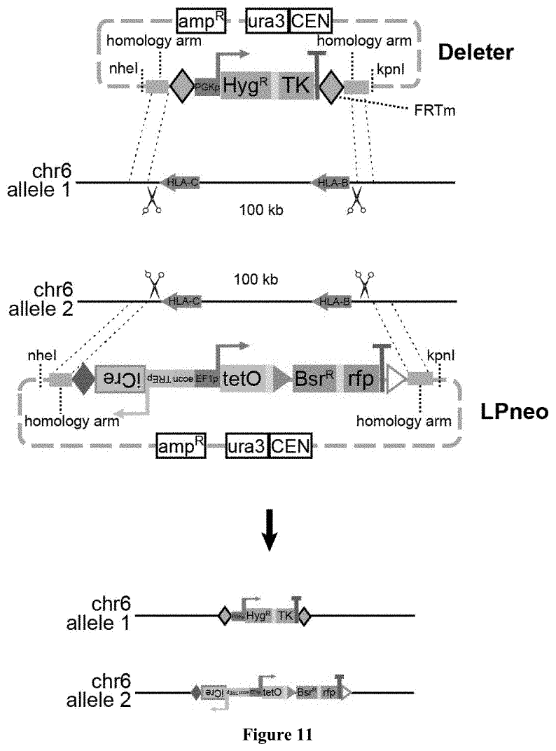

The second allele can also be deleted using a Deleter construct expressing Hygromycin resistance gene and Thymidine Kinase under a PGK promoter (FIG. 11). The Deleter construct was assembled by standard cloning methods into the same backbone as the REWRITE landing pad. It comprises an FRTm (F5) sequence, followed by the constitutive PGK promoter, a Hygromycin resistance gene, a P2A sequence, a Thymidine Kinase gene, an SV40 polyA sequence, and the another FRTm (F5) sequence. The Deleter construct is flanked by two alternative FRT sites (FRTm)—BsaI sites—used for golden gate cloning and insertion of homology arms for genomic integration, in the same way that is used by the REWRITE landing pad. The construct is co-transfected with the landing pad and uses the same reagents. Clones with bi-allelic deletions are selected using Blasticidin and Hygromycin simultaneously. The Deleter construct sequence is shown in SEQ ID NO: 15.

### Example 4: Designing and Building a Designer HLA-A/B/C (synHLA)

Big-DNA can be assembled up to 1 Mb by co-opting the naturally efficient homologous recombination (HR) machinery of budding yeast using techniques described earlier. Multiple linear DNA fragments can be stitched by using HR into a vector with yeast artificial chromosome (YAC) and bacterial artificial chromosome (BAC) replication sites, capable of carrying up to 1 Mb of DNA. Here the goal is to reconfigure HLA-A/B/C into a single locus of −115 kb (synHLA) or miniature version of ˜36 kb (mini-synHLA).

The HLA-A gene, including regulatory information (FIG. 5A), will be relocated centrally within the 78-kb intergenic region between HLA-B and C (FIGS. 5B-5C). The HLA-A core promoter maps within 1000 bp [52,53], and in silico analysis by Genehancer [54] (which integrates all public epigenetic, Hi-C, RNA expression, DNase hypersensitivity, eQTL data, etc) shows enhancers within just 2 kb of the gene body. To permit wild-type HLA-A expression, safety will be emphasized and the construct will encode up to 10 kb flanking region for the promoter/enhancer and 1 kb for the terminator in the design. This synthetic configuration enables single-step integration of HLA-A/B/C haplotypes into the blank iPSC, while preserving native regulatory information for cell-specific gene expression.

Both synHLA and mini-synHLA may be constructed from smaller 4-kb DNA pieces with −180 bp of overlap (genomic PCR of the iPSC DNA and/or commercial synthesis). Sections may also be captured through Transformation Assisted Cloning using yeast from readily available Bacterial Artificial Chromosomes (BACs) containing human genetic loci. Examples of personalized HLA genic DNAs (˜3.4 kb) will be commercially synthesized from an HLA haplotype database. The amplicons will be transformed into budding yeast with the linearized YAC/BAC vector, assembled by HR, and identified by selecting for the plasmid auxotrophic marker (e.g., HIS3) (FIG. 5C). Correct assemblies will be verified in yeast using junction PCR analysis and by deep sequencing. The 115-kb or 36-kb plasmid will be extracted from yeast and transformed into E. coli for DNA scale-up.

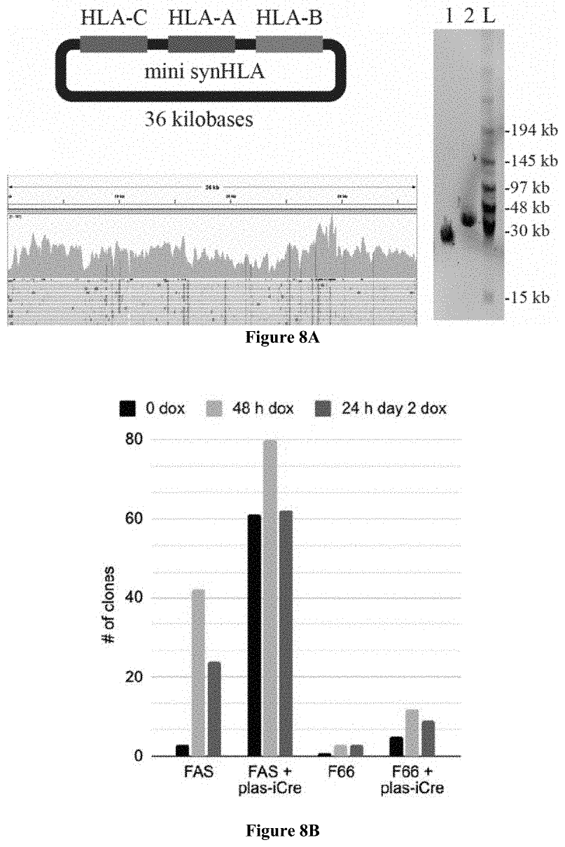

Using the methods described above, a mini-synHLA comprising HLA-A/B/C (FIG. 8A, top left) was constructed. Assembled mini-synHLA was analyzed by Gel Electrophoresis to confirm that it has the correct DNA size for the assembled mini-synHLA product (FIG. 8A, right) and sequenced using Illumina Miseq™ system (FIG. 8A, bottom left). Next, mini-synHLA was introduced to human iPSCs containing the REWRITE landing pad and inducible (by doxycycline) iCre via nucleofection. The iCre expression module was then activated by treating cells with doxycycline for up to 2 days to induce integration of HLA-A/B/C to the landing pad and the number of clones/colonies is shown in FIG. 8B.

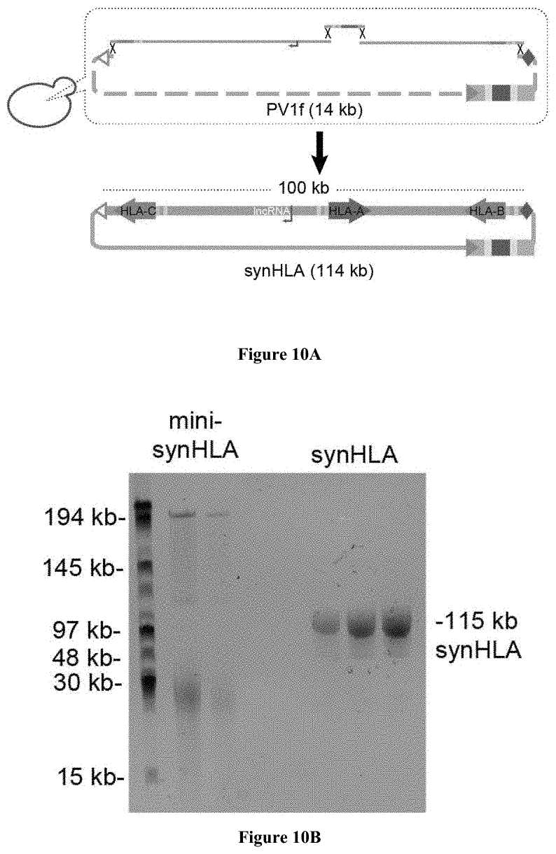

Using the methods described above, 115 kb synHLA comprising HLA-A/B/C was assembled in yeast. A 10 kB fragment of the HLA-A locus was placed within the 78 kb intergenic region between HLA-B and HLA-C from a BAC clone, and these were assembled into the Payload Vector (PV1f) in yeast (FIG. 10A). Assembled synHLA was analyzed by Field-Inversion Gel Electrophoresis to confirm that it has the correct DNA size and the size was compared with that of mini-synHLA (FIG. 10B).

### Example 5: Personalizing Blank iPSC with synHLA and Evaluating Expression

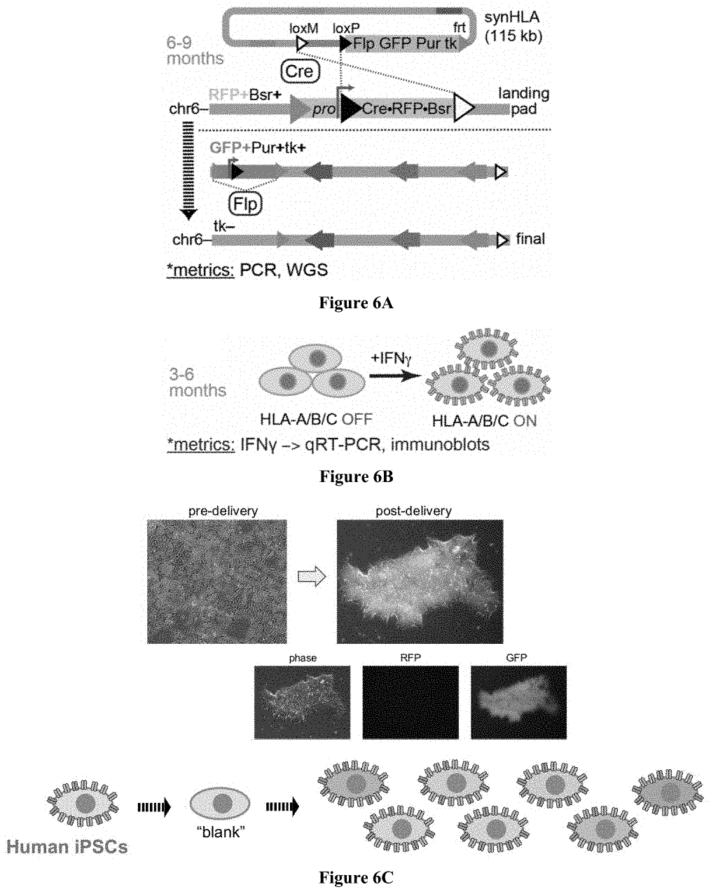

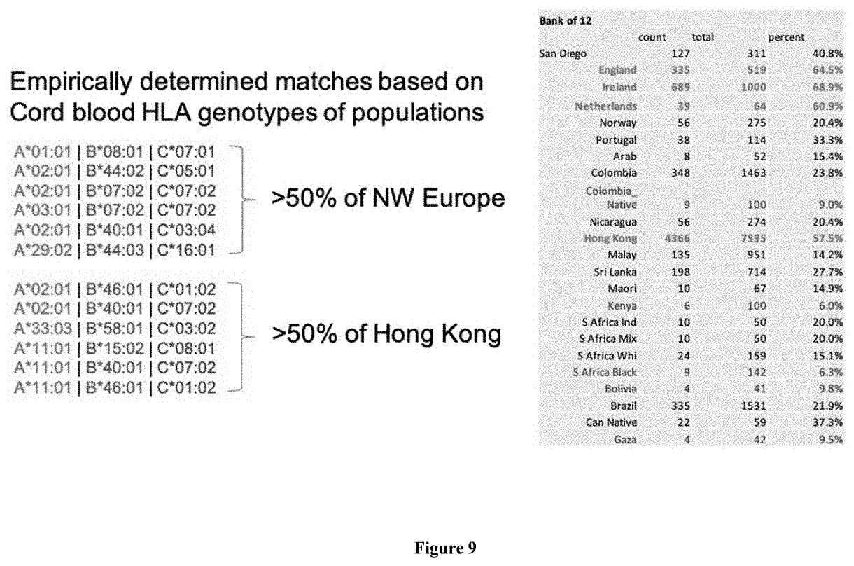

Personalizing class-I HLA haplotypes would provide immunological matches to various ethnic populations across the world (FIG. 9). To achieve this, a synHLA haplotype will be delivered to the blank iPSC using recombinase mediated cassette exchange method (RMCE) (FIGS. 6A-6C). The integration of 101 kb DNA has been shown using the related method Inducible Cassette Exchange (ICE) in mESCs [42]. The 115 kb synHLA will be delivered into an RMCE landing pad site within the blank iPSC. The RMCE strategy uses heterotypic loxP/loxM sites and an inducible site-specific recombinase Cre for “gene-trapping” as described earlier. The cassette is then excised using the Flp recombinase. The integrant will be verified using PCR and then confirmed of genetic integrity by 30x coverage whole genome sequencing (WGS). Then it will be tested to see if synHLA is expressed by adding IFNγ17 (PSCs don't express HLA), which induces HLA expression in iPSCs, to both personalized and wild-type iPSCs, and measuring the HLA expression by qRT-PCR and immunoblotting.

It is important to note that RMCE methods insert DNA without size-limitations, with high specificity, and reduce the need for whole genome sequencing or equivalent validation of each new cell line [25], as nuclease-assisted HR would require [26,27]. Only a single correct clone needs to be isolated as PSCs can be expanded indefinitely, therefore, high integration efficiency is not required.

Alternative methods:
1. synHLA can be retro-fit with ICE markers and delivered to pre-existing cell lines with the ICE landing pad.
2. CRISPR/MAD7 can be used to introduce synHLA by homology-directed repair.
3. synHLA can be randomly integrated to any locus as synHLA comprises all required regulatory sequences.

Authentication of Key Resources and Reproducibility
1. Human induced pluripotent stem cells (iPSCs): iPSCs were obtained from the Coriell Institute and the donor's race noted. The iPSC will be verified by karyotype analysis of chromosome number, microscopy, and whole genome sequencing at 30x coverage. A sensitive PCR test will be used to ensure that Mycoplasma is not present in the cultures. Finally, the cells will be grown on feeder-free conditions using commercial substrates (e.g., Matrigel, Laminin 521, etc), and prevented from spontaneous differentiation by including FGF2 or LIF.
2. Sex as a biological variable: The iPSC line in this work will be a male line. In future work, both male and female versions of cells will be included. This is because male cells have a sex-specific antigen (H-Y) that causes transplant rejection in females.
3. Reproducibility of experiments: The iPSC experiments will be carefully controlled to enable reproducibility. Temperature, humidity, liquid volume, and similar glassware will be used to reduce environmental variation during experiments. Furthermore, it will be noted which lots each batch of specific reagents (FBS, Media, Growth Factors, Antibodies) come from and these will be used uniformly. At least 3 biological replicates will be routinely performed for every experiment, and replicate experiment will be performed over time when possible. Finally, the use of commercial substrates for the iPSCs will ensure greater reproducibility compared to using a fibroblast feeder layer.
4. Other reagents: Examples described herein use standard biochemical and molecular biology reagents that are not expected to vary significantly. However, it will be noted specific vendors and batches for commercial reagents and the same will be used for internal controls to re-establish specific activities before using all reagents.
5. Control experiments routinely performed. All Big-DNAs will be fully sequenced. Standard controls will be used for immunoblots (tubulin and histones), karyotyping, and for qRT-PCR (housekeeping genes).
6. Statistical analysis. The experiments outlined in this proposal will utilize statistically sound numbers, usually experimental or biological replicates greater than three. Whole genome sequencing results will have at least 30x coverage, a standard in the field.

### Example 6: Preliminary Data Supporting Described Technology

Expertise Deleting Genomic DNAs (Example 3):

The inventors have technology to delete big genomic DNA regions to homozygosity, including the HLAs (H2K), from 5 kb to 200 kb in mouse embryonic stem cells (mESCs) at frequencies between 2-50%, without the use of any selectable marker (Table 3). These deletions were achieved using two targeted CRISPR gRNAs and a 100 bp single-stranded oligonucleotide (ssODN) to induce homology-directed repair [46,47]. In some embodiments, Inscripta's MAD7 CRISPR system can be used [48].

TABLE 3
Example mESC Genomic Deletions

| | Homozygous Δ |
|---|---|
| Gene - DNA size | Frequency |
| Major Histocompatibility Complex Class I, K | 50% |
| (H2K) - 5 kb | |
| Cluster of Differentiation 8 (CD8) - 55 kb | 20% |
| T-cell receptor beta (TCRβ) - 200 kb | 2% |

Expertise Building Big-DNA (Example 4):

Big-DNA assembly technology leveraging yeast homologous recombination and developed by the inventors enables fusion of multiple linear DNAs into one large Big-DNA with success in configurations so far up to 1 Mb [39]. A high-throughput automated workflow for the design, assembly, and verification (1536 qPCR and deep sequencing) can be used [43]. Assembling Big-DNAs in the HLA size-range is a routine task (Table 4).

TABLE 4
Example of DNAs generated

| Description | Organism | Length |
|---|---|---|
| Human biologic | Human | 88 kb |
| HPRT* | Human | 101 kb |
| Alpha globin | Mouse | 90 kb |
| HOX** locus | Rat | 170 kb |
| Chromosomes | Yeast | 1.4 Mb |

*HPRT: Hypoxanthine-guanine phosphoribosyltransferase
**HOX: Homeobox

Demonstration of 101 kb DNA Delivery to Mammalian Cells (Example 5):

Big-DNA assembly and delivery have been reduced to practice using Recombinase Mediated Cassette Exchange (RMCE). RMCE uses recombinases (Cre and others) to integrate large DNA payloads site-specifically. Mutant heterotypic recognition sites based on lox series (loxM, loxP, etc.) ensure unidirectional DNA insertion. A 101 kb DNA comprising the human HPRT locus has been integrated into mouse ESCs, as described previously [42] using a RMCE method called Inducible Cassette Exchange (ICE) [49]. A 170 kb DNA comprising rat HoxA locus has also been delivered to mESCs using the same method [62]. A 144 kb human Sox2 locus has been delivered to mESCs using “Big-IN” method [61]. ICE method can leave behind genomic scars such as transgenes used for positive selection.

Scarless DNA Integration with “REWRITE” (Example 5):

A robust and nearly scarless proprietary Big-DNA delivery method called REcombinase WRiting of Iterative DNA and Trap Excision (REWRITE), which leaves behind only 34 bp recombinase sites have been developed by the inventors. The REWRITE landing pad also co-delivers an inducible FLP recombinase gene for inducible excision of markers. This enables precise delivery of >100 kb DNA payloads (e.g., synHLA) without DNA scars (FIGS. 6A-6C).

### Example 7: Installation and Functional Validation of the REWRITE Landing Pad in U2OS

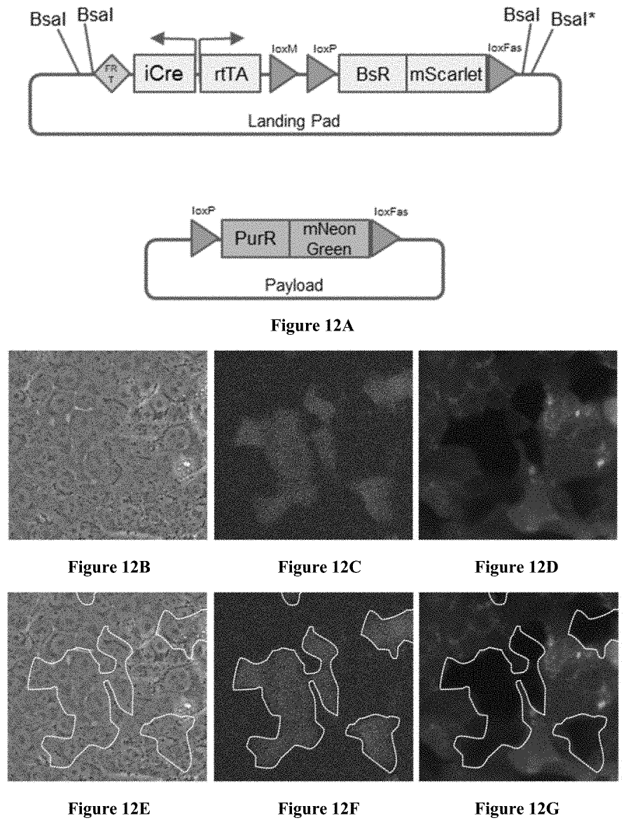

REWRITE landing pad plasmid and REWRITE payload plasmid containing location specific homology arms inserted at the flanking BsaI sites by Golden Gate Assembly were generated (FIG. 12A). As one of the BsaI site at the 3′ end of the landing pad can be methylated in bacteria, it is important to propagate the landing pad plasmid in Dam/Dcm−/− bacteria prior to adding homology arms. To test REWRITE recombination at a REWRITE Landing Pad, a REWRITE Landing Pad was installed in U2OS cells, and a minimal REWRITE payload (pDT3) was introduced to U2OS cells that had been treated with doxycycline and tamoxifen via transfection. U2OS cells were analyzed by fluorescent microscopy nine days post-transfection and (FIGS. 12B-12G). A field of U2OS cells were imaged in bright-field (FIG. 12B), GFP (FIG. 12C), and RFP channels (FIG. 12D). Groups of cells that have undergone REWRITE recombination with the REWRITE payload plasmid are outlined in white (FIGS. 12E-G). Cells that have undergone REWRITE recombination show expression of mNeongreen in the GFP channel (Figure F) but lack mScarlet expression in the RFP channel (Figure G). The sequence of the minimal REWRITE payload (pDT3) is shown in SEQ ID NO: 16.

The examples and embodiments described herein are for illustrative purposes only and various modifications or changes suggested to persons skilled in the art are to be included within the spirit and purview of this application and scope of the appended claims.

```
SEQUENCES
(HLA-B-A-C)
SEQ ID NO: 1
ATAACTTCGTATAGCATACATTATACGAAGTTATCCGGAACCGAGTACAAGCCCACGGTGCGCCTCGCCACCCGCG
ACGACGTCCCCAGGGCCGTCCGCACCCTCGCCGCCGCGTTCGCCGACTACCCCGCCACGCGCCACACCGTCGATCC
GGACCGCCACATCGAGCGCGTGACCGAGCTGCAAGAACTCTTCCTCACGCGCGTCGGGCTCGACATCGGCAAGGTG
TGGGTCGCGGACGACGGAGCCGCTGTGGCGGTCTGGACCACGCCGGAGAGCGTCGAAGCGGGGGCGGTGTTCGCCG
AGATCGGCCCGCGCATGGCCGAGTTGAGCGGTTCCCGGCTGGCCGCGCAGCAACAGATGGAGGGCCTCCTGGCTCC
GCACCGGCCCAAGGAGCCCGCGTGGTTCCTGGCCACCGTCGGAGTCTCGCCCGACCACCAGGGCAAGGGTCTGGGC
AGCGCCGTCGTGCTCCCCGGAGTGGAGGCGGCCGAGCGCGCCGGGGTGCCCGCCTTCCTGGAAACCTCCGCGCCCC
GCAACCTCCCCTTCTACGAGCGGCTCGGCTTCACCGTCACCGCCGACGTCGAGGTGCCCGAAGGACCGCGCACCTG
GTGCATGACCCGCAAGCCCGGTGCCGCATGCCCCACGCTACTGCGGGTTTATATAGACGGTCCCCACGGGATGGGG
AAAACCACCACCACGCAACTGCTGGTGGCCCTGGGTTCGCGCGACGATATCGTCTATGTACCCGAGCCGATGACTT
ACTGGCGGGTGCTGGGGGCTTCCGAGACAATCGCGAACATCTACACCACACAACACCGCCTCGACCAGGGTGAGAT
ATCGGCCGGGGACGCGGCGGTGGTAATGACAAGCGCCCAGATAACAATGGGTATGCCTTATGCCGTGACCGACGCC
GTTCTGGCTCCTCATATCGGGGGGGAGGCTGGTAGCTCACATGCCCCGCCCCCGGCCCTCACCCTCATCTTCGACC
GCCATCCCATCGCCGCCCTCCTGTGCTACCCGGCCGCGCGGTACCTTATGGGCAGCATGACCCCCCAGGCCGTGCT
GGCGTTCGTGGCCCTCATCCCGCCGACCTTGCCCGGCACCAACATCGTGCTTGGGGCCCTGCCGGAGGACAGACAC
ATCGACCGCCTGGCCAAACGCCAGCGCCCCGGCGAGCGGCTGGACCTGGCTATGCTGGCTGCGATTCGCCGCGTTT
ACGGGCTACTTGCCAATACGGTGCGGTATCTGCAGTGCGGCGGGTCGTGGCGGGAGGACTGGGGACAGCTTTCGGG
GACGGCCGTGCCGCCCCAGGGTGCCGAGCCCCAGAGCAACGCGGGCCCACGACCCCATATCGGGGACACGTTATTT
ACCCTGTTTCGGGCCCCCGAGTTGCTGGCCCCCAACGGCGACCTGTATAACGTGTTTGCCTGGGCCTTGGACGTCT
TGGCCAAACGCCTCCGTTCCATGCACGTCTTTATCCTGGATTACGACCAATCGCCCGCCGGCTGCCGGGACGCCCT
GCTGCAACTTACCTCCGGGATGGTCCAGACCCACGTCACCACCCCCGGCTCCATACCGACGATATGCGACCTGGCG
CGCACGTTTGCCCGCGAGATGGGGGAGGCTAACGAGCTCGGCAGTGGAGAGGGCAGAGGAAGTCTGCTAACATGCG
GTGACGTCGAGGAGAATCCTGGCCCAAGCCAGTTCGACATCCTGTGCAAGACCCCCCCCAAGGTGCTGGTGCGGCA
GTTCGTGGAGAGATTCGAGAGGCCCAGCGGCGAGAAGATCGCCAGCTGTGCCGCCGAGCTGACCTACCTGTGCTGG
ATGATCACCCACAACGGCACCGCCATCAAGAGGGCCACCTTCATGAGCTACAACACCATCATCAGCAACAGCCTGA
GCTTCGACATCGTGAACAAGAGCCTGCAGTTCAAGTACAAGACCCAGAAGGCCACCATCCTGGAGGCCAGCCTGAA
GAAGCTGATCCCCGCCTGGGAGTTCACCATCATCCCTTACAACGGCCAGAAGCACCAGAGCGACATCACCGACATC
GTGTCCAGCCTGCAGCTGCAGTTCGAGAGCAGCGAGGAGGCCGACAAGGGCAACAGCCACAGCAAGAAGATGCTGA
AGGCCCTGCTGTCCGAGGGCGAGAGCATCTGGGAGATCACCGAGAAGATCCTGAACAGCTTCGAGTACACCAGCAG
GTTCACCAAGACCAAGACCCTGTACCAGTTCCTGTTCCTGGCCACATTCATCAACTGCGGCAGGTTCAGCGACATC
AAGAACGTGGACCCCAAGAGCTTCAAGCTGGTGCAGAACAAGTACCTGGGCGTGATCATTCAGTGCCTGGTGACAG
AAACCAAGACAAGCGTGTCCAGGCACATCTACTTTTTCAGCGCCAGAGGCAGGATCGACCCCCTGGTGTACCTGGA
CGAGTTTCTGAGGAACAGCGAGCCCGTGCTGAAGAGAGTGAACAGGACCGGCAACAGCAGCAGCAACAAGCAGGAG
TACCAGCTGCTGAAGGACAACCTGGTGCGCAGCTACAACAAGGCCCTGAAGAAGAACGCCCCCTACCCCATCTTCG
CTATCAAGAACGGCCCTAAGAGCCACATCGGCAGGCACCTGATGACCAGCTTTCTGAGCATGAAGGGCCTGACCGA
GCTGACAAACGTGGTGGGCAACTGGAGCGACAAGAGGGCCTCCGCCGTGGCCAGGACCACCTACACCCACCAGATC
ACCGCCATCCCCGACCACTACTTCGCCCTGGTGTCCAGGTACTACGCCTACGACCCCATCAGCAAGGAGATGATCG
CCCTGAAGGACGAAACCAACCCCATCGAGGAGTGGCAGCACATCGAGCAGCTGAAGGGCAGCGCCGAGGGCAGCAT
CAGATACCCCGCCTGGAACGGCATCATCAGCCAGGAGGTGCTGGACTACCTGAGCAGCTACATCAACAGGCGGATC
ACCGGTTGCGTACGCGGATCCTCTGCTGGAGACATGAGAGCTGCCAACCTTTGGCCAAGCCCGCTCATGATCAAAC
GCTCTAAGAAGAACAGCCTGGCCTTGTCCCTGACGGCCGACCAGATGGTCAGTGCCTTGTTGGATGCTGAGCCCCC
CATACTCTATTCCGAGTATGATCCTACCAGACCCTTCAGTGAAGCTTCGATGATGGGCTTACTGACCAACCTGGCA
GACAGGGAGCTGGTTCACATGATCAACTGGGCGAAGAGGGTGCCAGGCTTTGTGGATTTGACCCTCCATGATCAGG
TCCACCTTCTAGAATGTGCCTGGCTAGAGATCCTGATGATTGGTCTCGTCTGGCGCTCCATGGAGCACCCAGTGAA
GCTACTGTTTGCTCCTAACTTGCTCTTGGACAGGAACCAGGGAAAATGTGTAGAGGGCATGGTGGAGATCTTCGAC
ATGCTGCTGGCTACATCATCTCGGTTCCGCATGATGAATCTGCAGGGAGAGGAGTTTGTGTGCCTCAAATCTATTA
TTTTGCTTAATTCTGGAGTGTACACATTTCTGTCCAGCACCCTGAAGTCTCTGGAAGAGAAGGACCATATCCACCG
AGTCCTGGACAAGATCACAGACACTTTGATCCACCTGATGGCCAAGGCAGGCCTGACCCTGCAGCAGCAGCACCAG
CGGCTGGCCCAGCTCCTCCTCATCCTCTCCCACATCAGGCACATGAGTAACAAAGGCATGGAGCATCTGTACAGCA
TGAAGTGCAAGAACGTGGTGCCCCTCTATGACCTGCTGCTGGAGGCGGCGGACGCCCACCGCCTACATGCGCCCAC
TAGCCGTGGAGGGGCATCCGTGGAGGAGACGGACCAAAGCCACTTGGCCACTGCGGGCTCTACTTCATCGCATTCC
TTGCAAAAGTATTACATCACGGGGGAGGCAGAGGGTTTCCCTGCCACAGCTGGCTCCGGCGAGGGCAGGGGAAGTC
TTCTAACATGCGGGGACGTGGAGGAAAATCCCGGCCCAGTGAGCAAGGGTGAGGAGGATAACATGGCCTCTCTCCC
AGCGACACATGAGTTACACATCTTTGGCTCCATCAACGGTGTGGACTTTGACATGGTGGGTCAGGGCACCGGCAAT
CCAAATGATGGTTATGAGGAGTTAAACCTGAAGTCCACCAAGGGCGACCTCCAGTTCTCCCCCTGGATTCTGGTCC
CTCATATCGGGTATGGCTTCCATCAGTACCTGCCCTACCCTGACGGGATGTCGCCTTTCCAGGCCGCCATGGTAGA
TGGCTCTGGATACCAAGTCCATCGCACAATGCAGTTTGAAGATGGTGCCTCCCTTACTGTGAACTACCGCTACACC
TACGAGGGAAGCCACATCAAAGGAGAGGCCCAGGTGAAGGGGACTGGTTTCCCTGCTGACGGTCCTGTGATGACCA
ACTCGCTGACCGCTGCGGACTGGTGCAGGTCGAAGAAGACTTACCCCAACGACAAAACCATCATCAGTACCTTTAA
GTGGAGTTACACCACTGGAAATGGCAAGCGCTACCGGAGCACTGCGCGGACCACCTACACCTTTGCCAAGCCAATG
GCGGCTAACTATCTGAAGAACCAGCCGATGTACGTGTTCCGTAAGACGGAACTCAAGCACTCCAAGACCGAGCTGA
ACTTCAAGGAGTGGCAAAAGGCGTTTACCGATGTGATGGGCATGGACGAGCTGTACAAGTGAATTCGATCATAATC
AGCCATACCACATTTGTAGAGGTTTTACTTGCTTTAAAAAACCTCCCACACCTCCCCCTGAACCTGAAACATAAAA
TGAATGCAATTGTTGTTGTTAACTTGTTTATTGCAGCTTATAATGGTTACAAATAAAGCAATAGCATCACAAATTT
CACAAATAAAGCATTTTTTTCACTGCATTCTAGTTGTGGTTTGTCCAAACTCATCAATGTATCTTATCATGTCTGG
CTCGAGGGAAGTTCCTATACTCTAGAAAGAATAGGAACTTCAAAGGAAGTGCTTCAGGGTGAAGGAAAATGATACT
AGATGGTGACCCAGATATACAGAAAGGAACAATTAACAACAGAAATGATGCACATACACATGTCACATTCACACTC
ATTTTCTTAATTTCCTGAAGACATATGGCTACTTGTTTAAAACAAAAAGTATTACACTGTATCATTGAGCTTATAA
TATAGGTTGATGTAATATATACAACAATAATAGCACAATGGTAGGTTACAAGAAACTACACTGTTAGAAGTGTCCT
TTATTTTCTGGATGCAGCTTAATATAACCTGAACTTCACTGTGAAAAGTCAAGGAATCGGGTTTCCATTCTTTGCT
ATTGTGTAAAGAAATATAGCCAAAAGCCACTAGGAGAATTAAAACCATAAACTAAAAAATGTTTATTTGACACATA
AGAAAGTAGTAAAGGAGGAACAGAAACAAAAAGATATGAGACAAATTGAAAACGTATAGCAAAATTGTAGACCAAA
ACCCAACCATTGCAAGTGAAGAAATGACATGACCTGAGTCACATTAGCAGGACTGCTGAGCACTGTGGGGAGAACA
GATATGGGCAGGAGGTGAGGGACAGTGTTAGTGCCACAATTCAGGAGGGAAAGGGTGGCAGGGACTAACGGGGGTG
GTGTGAAGGATGAGAAGGGCAGACAGAAGGGCTGGAGAGGCAGGAGGAGAGGAAAAGGAGCAGGAGAAAGAATTCT
AAAGCAGTGGAAGAGCCTGGCAGAGGGTTCTTTGCATTCGGTATTTAATACATTTTGTTGGACTTCCTAAAAACTA
ATTGGCTCCTTATGATTAAAAAAAAAAGAGTTACAAAAATACCAAGTGTTCAGATAAAATATGCACACTGCTTGGA
TGTGCAGAGTTCAGGAAAACAGGCAGTGCTTGAGCGCTGGTGAAGAGCATTGGGACTGCATGGAGCCCTCGCAACT
TTGAGGTGATGACTACAGGTTCCCGGTTGCAATAGACAGTAACAAACCCTGCTTCTTTATATTCAGGAGATGTTCT
GGACTCACACAGGGAAACTCAGGGTGGGGAATGAAGATAATTTTAAATGCAACAACCCAGAGTCACAGATCCATAG
TCTGGGAAAGTAAAACTTAGGAGCTTTGAGAGTTTAATTGTAATGCTGTTTTGACACAGGTCTTTTGCAGATTGGA
ATTCTAATCATTCAGGGATTACCAATATTGTGCTACCTACTGTATTAATAAACAAAAAGGAAACTGGTCTCTATGA
GAATCTCTGTGTGGTGGCTTCAGACAAAACTTCGCCAGGTTTAGAGAGAAAACCCCTGTCTCTACACCTCCATTCC
CAGGGCGAGCTCACTCTCTGGCATCAAGTTCTCCGTGATCAGCTTCCCTACACAAGANTCCAAGAGGAGAGGTAAG
GAGTGAGAGGCAGGGAGTCCAGTTCAGGGACAGGGATTCCAGGAGGAGAAGTGAAGGGGAAGCGGGTGGGCGCAGC
CTGGGGGTCTCTCCCTGGTTTCCACAGACAGATCCTTGTGCAGGACTCAGGCAGACAGTGTGACAAAGAGGCTGGT
GTAGGAGAAGAGGGATCAGGACGAAGTCCCATGTCCCGGACGGGGCTCTCAGGGTCTCAGGCTCCGAGGGCCGCGT
CTGCATTGGGGAGGCGCAGCGTTGGGGATTCCCCACTCCCACGAGTTTCACTTCTTCTCCCAACCTATGTCGGGTC
CTTCTTCCAGGATACTCGTGACGCGTCCCCATTTCCCACTCCCATTGGGTGTCGGGTGTCTAGAGAAGCCAATCAG
TGTCGCCGGGGTCCCAGTTCTAAAGTCCCCACGCACCCACCCGGACTCAGAGTCTCCTCAGACACCGAGATGCGGG
TCACGGCACCCCGAACCCTCCTCCTGCTGCTCTGGGGGGCCCTGGCCCTGACCGAGACCTGGGCCGGTGAGTGCGG
GTCGGGAGGGAAATGGCCTCTGTGGGGAGGAGCGAGGGGACCGCAGGCGGGGGCGCAGGACCCGGGGAGCCGCGCC
GGGAGGAGGGTCTGGCGGGTCTCAGCCCCTCCTCGCCCCCAGGCTCCCACTCCATGAGGTATTTCTACACCGCCAT
GTCCCGGCCCGGCCGCGGGGAGCCCCGCTTCATCGCAGTGGGCTACGTGGACGACACGCAGTTCGTGAGGTTCGAC
AGCGACGCCGCGAGTCCGAGAGAGGAGCCGCGGGCGCCGTGGATAGAGCAGGAGGGGCCGGAGTATTGGGACCGGA
ACACACAGATCTACAAGGCCCAGGCACAGACTGACCGAGAGAGCCTGCGGAACCTGCGCGGCTACTACAACCAGAG
CGAGGCCGGTGAGTGACCCCGGCCCGGGGCGCAGGTCACGACTCCCCATCCCCCACGTACGGCCCGGGTCGCCCCG
AGTCTCCGGGTCCGAGATCCGCCTCCCTGAGGCCACGGGACCCGCCCAGACCCTCGACCGGCGAGAGCCCCAGGCG
CGTTTACCCGGTTTCATTTTCAGTTGAGGCCAAAATCCCCGCGGGTTGGTCGGGGCGGGGCGGGGCTCGGGGGACG
GGGCTGACCGCGGGGCCGGGGCCAGGGTCTCACACTTGGCAGACGATGTATGGCTGCGACCTGGGGCCGGACGGGC
GCCTCCTCCGCGGGCATAACCAGTTAGCCTACGACGGCAAGGATTACATCGCCCTGAACGAGGACCTGAGCTCCTG
GACCGCGGCGGACACCGCGGCTCAGATCACCCAGCGCAAGTGGGAGGCGGCCCGTGAGGCGGAGCAGCTGAGAGCC
TACCTGGAGGGCACGTGCGTGGAGTGGCTCCGCAGATACCTGGAGAACGGGAAGGAGACGCTGCAGCGCGCGGGTA
CCAGGGGCAGTGGGGAGCCTTCCCCATCTCCTATAGGTCGCCGGGGATGGCCTCCCACGAGAAGAGGAGGAAAATG
GGATCAGCGCTAGAATGTCGCCCTCCCTTGAATGGAGAATGGCATGAGTTTTCCTGAGTTTCCTCTGAGGGCCCCC
TCTTCTCTCTAGGACAATTAAGGGATGACGTCTCTGAGGAAATGGAGGGGAAGACAGTCCCTAGAATACTGATCAG
GGGTCCTCTTTGACCCCTGCAGCAGCCTTGGGAACCGTGACTTTTCCTCTCAGGCCTTGTTCTCTGCCTCACACTC
AGTGTGTTTGGGGCTCTGATTCCAGCACTTCTGAGTCACTTTACCTCCACTCAGCTCAGGAGCAGAAGTCCCTGTT
CCCCGCTCAGAGACTCGAACTTTCCAATGAATAGGAGATTATCCCAGGTGCCTGCGTCCAGGCTGGTGTCTGGGTT
CTGTGCCCCTTCCCCACCCCAGGTGTCCTGTCCATTCTCAGGCTGGTCACATGGGTGGTCCTAGGGTGTCCTATGA
GAGATGCAAAGCGCCTGAATTTTCTGACTCTTCCCATCAGACCCCCCAAAGACACACGTGACCCACCACCCCATCT
CTGACCATGAGGCCACCCTGAGGTGCTGGGCCCTGGGCTTCTACCCTGCGGAGATCACACTGACCTGGCAGCGGGA
TGGCGAGGACCAAACTCAGGACACTGAGCTTGTGGAGACCAGACCAGCAGGAGATAGAACCTTCCAGAAGTGGGCA
GCTGTGGTGGTGCCTTCTGGAGAAGAGCAGAGATACACATGCCATGTACAGCATGAGGGGCTGCCGAAGCCCCTCA
CCCTGAGATGGGGTAAGGAGGGGGATGAGGGGTCATATCTCTTCTCAGGGAAAGCAGGAGCCCTTCTGGAGCCCTT
CAGCAGGGTCAGGGCCCCTCGTCTTCCCCTCCTTTCCCAGAGCCATCTTCCCAGTCCACCATCCCCATCGTGGGCA
TTGTTGCTGGCCTGGCTGTCCTAGCAGTTGTGGTCATCGGAGCTGTGGTCGCTACTGTGATGTGTAGGAGGAAGAG
CTCAGGTAGGGAAGGGGTGAGGGGTGGGGTCTGGGTTTTCTTGTCCCACTGGGGGTTTCAAGCCCCAGGTAGAAGT
GTTCCCTGCCTCATTACTGGGAAGCAGCATCCACACAGGGGCTAATGCAGCCTGGGACCCTGTGTGCCAGCACTTA
CTCTTTTGTGCAGCACATGTGACAATGAAGGACGGATGTATCACCTTGATGGTTGTGGTGTTGGGGTCCTGATTTC
AGCATTCATGAGTCAGGGGAAGGTCCCTGCTAAGGACAGACCTTAGGAGGGCAGTTGGTCCAGGACCCACACTTGC
TTTCCTCGTGTTTCCTGATCCTGCCTTGGGTCTGTAGTCATACTTCTGGAAATTCCTTTTGGGTCCAAGACGAGGA
GGTTCCTCTAAGATCTCATGGCCCTGCTTCCTCCCAGTCCCCTCACAGGACATTTTCTTCCCACAGGTGGAAAAGG
AGGGAGCTACTCTCAGGCTGCGTGTAAGTGGTGGGGGTGGGAGTGTGGAGGAGCTCACCCACCCCATAATTCCTCC
TGTCCCACGTCTCCTGCGGGCTCTGACCAGGTCCTGTTTTTGTTCTACTCCAGCCAGCGACAGTGCCCAGGGCTCT
GATGTGTCTCTCACAGCTTGAAAAGGTGAGATTCTTGGGGTCTAGAGTGGGCGGGGGGGGGTGGGGTGGGGAGGGG
GCAGAGGGGAAAGGCCTGGGTAATGGAGATTCTTTGATTGGGATGTTTCGCGTGTGTGGTGGGCTGTTTAGAGTGT
CATCACTTACCATGACTAACCAGAATTTGTTCATGACTGTTGTTTTCTGTAGCCTGAGACAGCTGTCTTGTGAGGG
ACTGAGATGCAGGATTTCTTCACTCCTCCCCTTTGTGACTTCAAGAGCCTCTGGCATCTCTTTCTGCAAAGGCACC
TGAATGTGTCTGCGTCCCTGTTAGCATAATGTGAGGAGGTGGAGAGACAGCCCACCCCCGTGTCCACTGTGACCCC
TGTTCCCATGCTGACCTGTGTTTCCTCCCCAGTCATCTTTCCTGTTCCAGAGGGGTGGGGCTGGATGTCTCCATCT
CTGTCTCAACTTTATGTGCACTGAGCTGCAACTTCTTACTTCCCTACTGAAAATAAGAATCTGAATATAAATTTGT
TTTCTCAAATATTTGCTATGAGAGGTTGATGGATTAATTAAATAAGTCAATTCCTGGAATGTGAGAGAGCAAATAA
AGACCTGAGAACTTTCCAGAATCTGCATGTTCGCTGTGCTGAGTCTGTTGCAGGTGGGGTGTGGAGAAGGCTGTGG
GGGGCCGAGTGTGGACAGGGCCTGTGCCCAGTTGTTGTTGAGCCCATCATGGGCTCTATGTGGTTAGTCCTCAGCT
GGGTCACCTTCACTGCCCCATTGTCCTTGTCCCTTCAGCGGAAACTTGTCCAGTGGGAGCTGTGACCACAGAGGCT
CACACATCGCCCAGGGTGGCCCCTGCACACGGGGGTCTCTGTGCATTCTGAGACAAATTTTCAGAGCCATTCACCT
CCTGCCCTGCTTCTAGAGCTCCTTTTCTGCTCTGCTCTCCTGCCCTCTCTCCCTGCCCTGGTTCTAGTGATCTTGG
TGCTGAATCCAATCCCAACTCCTGAATCTGTAAAGCAGAGTCTAATTTAGACTTACATTTGTCTGTGAAATTGGAC
CCATCATCAAGGACTGTTCTTTCCTGAAGGGAGAACCTGATTGTGTGCTGCAGTGTGCTGGGGCAGGGGGTGCGGG
GAGGGGGTTGCTGTAGAAAGAGGGATGGGGAGGGATGGCCTGAGGCTATGTTAATAAAGATACTGCCTTCAAAATA
GGGAGGTGCTCTACAGTGATCATTCATTCAACTGACCTTTGTCATTGGCCAGACATAGGACAGAATGGTTCTGCAT
CTGGGGAACACCACTGAAGTAAAATCAGAAAAATCTCTGGCCTTTTGTAGCATATGTTCCAGTGGGAAGAGGCAGA
CGATAGATACACTATAACCAGAGTAAGGAAGGAAAGTGCTAGAAGGTGGTAAGTGCTGTGAGGCAGGTGATCCAGG
ATGTGGGCAGTGGGGACAGGGAAGGTGGCTGTTGTGCTGGGTAGTCAGTGTGTGCCTTGTTGCAAAGGTGACTTTT
GAGGAAAGATTTGAGAGACATGAGGATGTCTGGGGAAGTTCTTTCCAGGCAGAGGAAGCTCCAGTCCAAATGCACT
ATGGCAGGAAGGTGTTTGTGTTCCCAGAAGAGCAAGGAGGCCAGGAGGGCTGGACGGAGAGAAACTGAGGTGAGGT
CAGAGGTGCGGCCAGAACAGGTGGGCTTGAGGGGAGTGGGGTTGGATCTGGCCTTTGCTCTGAGTGGGATGGGGAG
TTAGAGGACAGTTTTGAGCAGAAGAGAGCCATGATATGACTTCTGTTTTAAAAGGATCTCTCTGACGGCTGTGCTG
AGAACAGAATTGAGAGGCAAGGGACGAGGGAGGCAGAAGGGAAAACAGTAGGAATCAAGTGCAGTATTCCAGGCTG
GAGATGTCGGTTACCTTGACTGGGGCGTGAGCAGGGGAAATAGTGGGACGTGAGGGGATTCTGGATGCATTTGAAG
ATGGACTCACAGCATTTGCCAATGGATTGTATCTGTGATGTGAGAAAGACGAATCAAGGACACCCATAGTTGTAAA
ATGAGTGAGTAGAAGGATGGAGCTGCTGTCAGTGGAGATGGGGAGACTCTGGCAGGAGCGTCCTGAAGAGGGGACA
TCACAGGCACTCAGTGGAGGAGATGTCTACTAGGAGGGCGGGTGGGGGAGCTGGGTGGAATTTGGACAGACAACTC
CAGAGTTTAGGGGAAAGGACTGGGCTGGAGAAATAGATTTAGGAGGTCACACCATATATATGAGACTTAAAACCTC
AAGCATGGATGAGGCACAAAGGGAGTGACTGACTATGGAAAAGAATGAGCACAAGGACTGAACCCTGGACCTCCAG
TTCTAAGGGGTGTGATCAGATCACACCCAGAGCAGACTGCACAGTTCTGGCCCCATGTCTAGAGGACACTCAGACA
AGGAAACCCCATGTGCACCAGGATCATCTGGATGTGGTGCTGAGATCCTGGAAGTCTGGAGTTGAGCAAGAGATTC
TGGATTTATGACAAGGCTGGAGCTCATGTTGCTGGTCTCCAGATCACACTTGGAGTAGCAAGAACACCAGGATCCC
ACACGTCTGAGCATCAGCCTCACCTCTAGGGCTTGTCATATAAATGATTCCTTGGTCTTGTGCATAATTCTCTGAG
ACAAGGGTTCTGGGGAGTGGCCTGTGTATTTTCTAAGTCCCCACCAGCAATCCTATTGCTCAGACAGATTGGGAGC
CACTGAGATCAGTGATCAGAGAGTGCCCAGGGTGGGTGGGTGGGGTGGGTTTTCAAACCCTGTTGAAAAGAGGATT
TTTCTCACAGAAAGAAAAGGGAGGATGTATATCATCAGTTATGAGAGGTGATATTCTCTGTTGTTCTCTCCACCAT
GGGGTAGAGGCCAGGTAGACAATTCAGGATGTGGCTCTCGCACAAAGAACACCTCTGAATGCCGCTCTCTGACACT
CGCCCGCAGACTCATTTCTCACTCACTTCTTGGAGAAAACTATGGAAACCAAATTTCTGTAATGTACACATAAAGT
TGTATATTGGTATTGGGGGCTAGTTTTATTGTGGGGAAGGCCACAGAACCAGGCTGGAAACTACACATCCAAGAAC
AGAATTCACAGCCCACCCTGGATCAGGTCCCTCCTAGGAACAACTGCCCCTGCTGCTGAGCACAGACAGCACTGCT
CACACCTCTGACATCCTGGTGCTGGACACTGGACCCTGAGGCTAGGACAGATGTCACTGCTGCACCTGGAAATTGG
ACATCACTACTGTCACTCAGCCATCTTTACTAAAATGCTTTCTGCACAGTCCTAGCCTCTCTGTCACCTCATTCTG
GCTGAGTCTGGCAGTGATATAGGAGTTAAGAAGAAATTATTTAGACAGGTAGTGAGGGTACGCAAGGCTTTTAATG
GAAAGCAGGCTCCAAATTATTTTCTTTTCTAACAAGGAGCAGCCTGTAATATCAAGCTTCAGACATAGACAAGTAA
GCTGGAAGTTTGCACGGGTGAATGCCAGCAGCTGTCCAATAGGAATAGGACGCCTGGGACTGGGCATGTTCACTAT
GGCGGCTCCATCTTCCCTTCTCTTTGCCAGGCACGTTTATAGTAAGGAGCAGACAACATGTCACCGGCCAAGTGGA
AAGCCCATTTGCATAATAGGATGAGGGTGGGGTGAACAGCCTTCCACACGCACTATGTAAATATCACACCTGGTAC
AACCAACCTGTGGGCCCTACATAAATCAGACACCATCTCCTCAAGCCTGCCTGTAAAATCCGATGCACTCCATTCC
AGGCTGGAATTCCCTTTTGGGTGCCCCTCTCTCTCACAAGAAGGAGCTGTTTCCCTTTGTCTTTCTTTTGCCTATT
AAACCCTTTGATCCTAACTCACTCCTCGTGTGTCAGTGTCCTTAATCTTCTTGGCGTGAGATGACGAACACTTAGT
ATTTACCTGAGACAATGACACCTCTTCAGCAGGTGGTCATCAGACTGGTGGAGCTTAAAATTCATGTCCATGATCA
ACTGCAGGGGTGACTGGAGTGTTGTTGTCCTCTTCCAGGGAGGGAGGTGGTGTCTGTCTCCTCTCAACATTTTACA
TGTTGTAAACCAAAAATAAACATCAAAGCCACCCCTTCCCAATCATTTTAATGGACACCCTCAGCCAGGGTGCTCA
AAAGTTAACCTGAAAGAGTGGCTCAGGCCACCATGGGAAGCAGGTATTGAACATGTCTTATTATGCCCTCTTCCCT
CTTGGAATTCAGGAAAAGTTGACCAGCATTTACCATCAACACAGACCTTCAGTCTGATCAGAAACATTTACAATCT
ATTCTCTTTGAAGCCTGGCACCTGGAGGCGTCATCTGTATGAGAATTTTGGACTCCACAACCTTTTATCATAACCC
AGACATTCCTTTCTATTGATAATAACAACCAATTGCCAATCAGAAAAATTAAAAATCTACTTATAACCCAGAAGCA
CTACCCCGCAACCCTTGCTTGCTTCAAATTGTTCCAACTTTCTGGACCGAACCAATGTATAGCTTAAATGTATTTG
ATTGATGTCTCATATCTTCCTTAAATGTGTAAAACCATGTATTTGATTGATGTCTCATATCTTCCTTAAATGTGTA
AAACCAACCTGTGCCTCAACCACCTTGAGCACATGTTCTCGGTCTCCTGAGGGCTGTGTCATGGGCTGTGGTCACT
CATTTTTGGCTCAGAATAAATCTCTTTAAATATGTTACTGCGTTTGACTCTTTTCATTGACAGTGTGGAATTTTGA
TGTAGTAAGCGGGTTCAAGTGCTGGAGTGTGAGGGGTGGGGAAAAGTGACAAATTTTAATTCTTGGAGCAGTGATC
CAGGACGAGAACTTTATCTAGTACCCAGTAGGCACGAAGAGTGGCTGAATGAACCAGTGACTAATAAATAACATAT
TTCCACCCATTCCCTTGAGGATAGGTTTACATCAAGTTTTTGTCTATCCCAGTTTATAGTCTAGATTATTGGAGTG
GCATTCTGTAATGATGAAATGATTATTCAAACCTCTTCCCTCAGCATGACTCTTACTAGGATTAAAACTCCCAGGT
TTGTAAGATATCAGAGGTTCTGGTTCCACACGATGTTTTATATTAATATTTATATTTTCTTGTGTGTGGAAATATT
TCTGGGGAGAGAATCTCTAACATTTATTAGTTTTTATGTGACACCCTCAAAGGCCTAAAAAATCCTAACTTCTAGA
TTAAAGGTAGGACCACTCCACGTCTAGACAATGAACAGCTGGTAGCAACCTGTGTTTAGGGACATGAAACAAATGC
TTACATTTGTTTATCTTTTCCTCTGGGACCAAAGAAGGTAGAGGTTTACTTGCTTACTCTTACTATAAGTTTCTCT
CTGTGGGATGGTGATGGGGTTTTCTTCCACCCAAATAACTTTCTGATTCTCCAACAGCAGCTGGGAGTCCCACAAT
TTGACTCAATTCTGACACTAACTACCTGGAGTTCGCCTCAGACTCTACAGGTTTACCAGCTCAGTCCTACAAGTCT
GTCCTCACTTCAGAAGCAAGTCACAAGTATCGGGTCCCAGGTTACCTGCACTTCTGTCTAACATGGCTGCAAAGTC
AGGGGTTTCTCACCTCCCAAGGATTGAGAATTCTTAAACTAACTCAAAGAACTCAGGAAGTTGCTATAGCCGTGTA
AACCAAAAAACGTCTGAGACAGGTCTCAGTCAGTTTAGAAGTTTATTTTGCCAAGCTGAAGACGCACCTTGGAAAC
AGAGACACAAATCATTGTAGCATCTGTGGCCCATGCTCTTTCCTAACAGGCTTTTGAGGACTTCAATATGTACAGG
GGAAAGAGCAGCCAGGAGAGGAAAAAAATAGTCAACTATGCATTCATCTCATGCTCAGTAAATCTGCATTTTAAAT
AAGATAAAGTAAACATTGGGTGGAAAAGTCAAATATGCGTTTGACTCTGGGTGGGTGGAGGGATGATTTCTAGTCT
TGTCTTTGTCCTGTACCTGTGAAGATAAGCTGTTAATTTACATTGTCAGGGGAAAATTCAACAAAACTGTTTTAGG
GTAAACACGTTGGGGCCCACAAGGAATTATCTTGTGAGCAGTTTGTGAGGGAGGCCACCTGGGGAGATATGTGGTC
TTCTATCTTTGCAGTTATTTGTTTAGGAACGAAAGAAAGGCAGTTTTTCCATCAGTCTCCAAACTTAATTTTTCCT
TTTGGTATAGTGAATTTGGAGTCCTGAAATTTTATTTTCTTTTCACACTCAGTATTGCAGTTTTTTATTGTTATAG
CTTAAATCCCCACCTTTTTGGCACCTGGGACCGGTTTCATGGAAGACAGTTTTTCCATGGACTAGTGGAGGTGGTG
GAGGGCAGAGGGAAATGGTTTCGGATGAAACTGTTGTACCTCAGATGATCAGGCATTAGCGCTTCATAAGGAGCAT
GCAACCTAGATCTCTCGCATGCAGAGTTCACAATAGGGTTCGTGCTTCTATGAGAATCTAATTCCGCCACTGATCT
GACAGGAGGCGGAACTCTGGTGTCATGCTCATGCTCTCTCACCCACTGATCACCTCCTGCTGTGCAGTCTGGTTCC
TACCAGGCCAGGAACCTGTACCAGTCCGCAGCCCGGGGGTTAAGGACCCCTGGTTTAAATTATGCCACTAGAGCAG
AACTCCTGAGACTGGATAACTTATAACAAACAGAAATTTGTCTCGTTCATGGTGTCGAGGATGAGAATTCCCAGAT
CAAGGGACTCTATCTGGTAGAAAACAAAAGGACAAGAGAGGGTGAGAGGTGGGGGGAAAAGGAAGTCAAACTCACA
GCCTCAGGTCCTTTTATCATGAGCCTTAGTCCATGAATGAAGGCAGAGCTCTGATGGTCCAATCACCTCTCAAAAG
TCCCATTTCTTAAAAATGTTGCATTTGGGATGAAATATCCAACAGATGCTTTTTTGGGGAAACATTCAAACCATAG
CAGTTATAAAGGATACCAAGGAGCAGCCAGATGAAGAGGTGCACAGGGTGAGGCCTGGAAGGGCTCTGAGCACAGG
AGCCTCTGTCCCCATGGAGATGGGGCTCACCATCCTCCCAGCATGTGGACACACAGATGTGCTCTCCGAAACCCAA
TCTCCAGAGGTTTCTATGGAAATTTCATTGTGGAGGCAAGACTAATTAAATCACTGGTCATTGTGTGACTGAACTC
AACCTCGAACCTCTCTTTCCTTCCCAGACTTTAGGGATGGGGTTAACAGTTCCAATCCTCTAATCACAACTTCGTG
TTTCTGGCAACCAGCCCCCATCCTGAAGCTATCTAGGAGCCCCTCACCCCATAGTCATCTCATTGCCATATTGAAC
TCATGTTTATCACTGAGGAGATTGCAAATGTTTTAGAACCTGGTTGTCAGGAAACAGGGAAAAGACCAAATATTTT
TATTGGACCACAGATCACACCCTGGTTTTGACCACATTATCTCTTTTTATAATATTTATTATTTTATTTTAGAGAT
AATCTCTTGCTCTGTTGCCCAGGGTAGAATGCAGTGGCACAATCATAGCTCACTCTAACCTCGAATTCCTGGACTC
AAGTGATCCTTCTGCCTCAGCCTCCTGAGTAGATAGCATTACAGGTGCACACCAACATGCCCAACTAATTTTTAAA
TATTTTGTAGGAATGGGTCTCACTATTCTGCCCAGTCTGGTCTCAAACTCCTGGACTTAAACAATGTTCCTACCAT
TGCCTCCCAAACTGATGGAATTATAAGTGTGAGCCACTGTGCCTGGCCAATCACAGATCTCCTACACCAGAAGAAT
CATAACAGTGAAAAGATCCTGGCACATTACCAGAATCCTCTTTGGTCATTAACAATGATCCCAGTGCAATCCATAA
ACAGGAATAGTTTCAGCAACATATGGCTGCACCCTTTCAGGCGTCTGGTAAAATTGAGCTAAGAGACAATATCATC
TCTTGCTTATACCTTTTTCAAGTAGTTCATGTAACATTGGATTTTCCTCATTACATAACCCATTTATTCATTCATT
TACCCTCAGCTACTATTTCTCCTTCCCTTCATTTATACTAAACATTTATAATTTTGGAAGGAACACTAGCTTCTGC
TGCTGTGCTGGCTGAGACTGCAGGGACCAATAGAGTTCTAGCAAGTGTCTCCCGTCATCCATTCCAGTTCATAGAG
GGTAGGGTTATACAGGTAGAGAACTGGTTGGCTATCTGATCCCAGGCAATATAGCTGCATTCAGTGTTAGCCCCAA
CTTTGCCAGATGCAGTGAAGGCACAACCTACTTCTACAGGCCCTTAGGAATTATTACATAATGGACATAAAATATA
TTTACAGTTTCTTGCTTAGGAATAATTCCTGTTTCTGAACTCTATTTGCATCCCTAGTCCTGGGACCACTGCATCA
GGTATGAGAAAAGCAAGTTGAGGTGAATTACAGTCATCATTCCAGTGTCCTCCCACCTTGGGGAGGAATATATGTA
GATCATACCAGCACCTTCCCCTGATCCACCAGGAAAAGAGAGGATACTCTCATGTCAAGTGTGAGCACACTCATGA
AGGTGTATAAGCCCACCCTTTGTGCTTGTTTCCCCACTCTTTCCTTTACACACCCAATTGACTATTTTAATTCTCT
ATGGAACTCTTCCTCTGAGGAGGATATCTCTCTGTGACCATTGTTGGACATTTTGAGCAGGTGCATTTTCTGGTGT
AAAGAAATATGACTCGGTGGTCCACGTGGTTGCAGTACCTCCCATTCAAGTTCTTTTGTAACACTCGAGCATTTGC
ATCTGAGATAAAGCAGGGACCTCTTTTAGAAGCCTGCCGGGGCCTCCCAATCTGGAAATAAAGAAAAACCTTCAGT
TCCTTCAAGAGAAATTCCAGGCACCTAGCCAGCCCTAAAAAGTAAGTCGGTGACCTTATAAGCAAGAAGGAATGAT
AGCTTGAAACAATAGCCAAGGAAGTTAGAGTCACAAGATGTTTCATTCTCTATAGAAACTAAAAATAACATCTTAA
CATATGTCCCTGAGTTGTTTTTCAGAAACATGGATTCCCTCCTAATAGATCCACTGACAGGGAGACCTTATAAAGG
GGAAACTGAGAACTGAACTCTGACCGCCGTTCTTTGTTCTAAATTTCTTCCTGAGGGACCTGGAGAGAGTCAAGCC
CACAGGCCAGGCCTGAACATTCCTCTCTACTGCCCCCAACTGTGTAAGGAAGCTGTGTAAAGAAGCTTTGCTTCCT
TATCTATCACAAATCAGAGAATCTTTAAATCTACCTAAGACCTGTAAGCTCCCTCATCAGGATATTCTACCTTTTA
AGGCCAAAGCAGTGTAACTTGTATGCATTGATTTATGATGTTGCCTGTAACTCTGCTTTCCCGAAATTTGCCCCTG
CCTTTAAAAATTCTTGCTTGTGAGCCATCAAAAANGTTGGGTCTTAAGCGTGAGCTGCCTGATTCTCCTTGTTTGG
CGCCCTACACATAAATGCCCTCCTTTGTCCCACTACAATCTCAGTGTGGATGTTTGCTTTTACTGCCCCAGGTGAG
TGGACCCCAGTTCAGTTTGGTAACACACCTACCACCAGCTGAACAAAGCCCACTCCAGGGTGAGGATCTATTCCTG
CCAAGACCCGTTTGCTGCTTCCTGGGGCTACTGGTATCAGTCTAACTTGCCAGCTATGTATTTTCAAGGCCCTCCC
ATAAAAGAATCTGCCACATAGCCATATGCTATCTCTCTTTTTTTTTGTTAAAGAAAGGACTTCTTATGTGTATTTT
GTGCCTGTGATGTGTGGAATGCAAGAGGACTATGTCTTGATTCCACCTATCTCTGCATTGCTACAGCCCCAATGTC
TACTTATTTCATGGACTCAGGTGATCACCACAAGCAAACCTGGATTTTTTGTTGTTGTTGTTTTTGAGATGTGGTC
TTGATATGTTTCCCTGGCTGGCCTCAAATGTTGCTCAGGTTAAACTCTAACTCCTGGACTCAAGATATCCTTCTGC
CTCAGCCGCTCTCATAGCTGGAATTACAAGTGCACCTGGCAAACACTTGGAGATAAATTCTTGTTGATTTCAATCA
CTTTCCAAACCTGAAAGGGAGTTATTCTGAAGGACACTGAACAGCACTGAGGGGACGGTGCTAAACCATTCATGAG
AAACTGACCTCGTGATCCAACCACCTCCCTCCCTCCAGGCCCCACCTATAATGCTGGGGATCACATCTCGACATGA
GATCTGCACAAGAACAACATCCAATCTATATCAGGCATCAACAGATCTTACATTAATGAACCCCTCAAATTACCAT
AGTGATTTCTGTGGGACTTGGCTCATATGGGCATTCTTTTCATAGGCAAGTTTTTCATTGCTTTCCTGATTAATTA
TACGGCCAGGCCATTAGTCACTCCCTGTAAGTCAGTAAAAACTAAAACGTAGGAGCTTTATCATTGTTCAATTCTT
CCACCACTGCTAAGAAAACACCATGCAATTAAGTCCACAACAGCTTTTCTGTTTTTACCTTCTTTGATCAAAGAGG
TGGACTTCCAAACAGTATGTTGTCCATTCATCTTGCAAATGCTGTACACAAACCAACCAGCTGTTAGTGGGTCAGT
TGACTGATACTGCAGTTCAAATCCCAGCGCTGTCTGCTACAGAGTTCCATCTTGCTCAAGAGAGGTCAGTGTTTTG
TTTAATTTACACCTTCAGTGAATTGAATGAAGCCCACTCACATTATAGACATTAATCTACTTTATTCAAAGTCTGG
CTATTTATGTGTTAATCTCATCCAACAAACATCCTCACAAATCATCCAGAATAATGCTTGACCACCTCTCCAGGCA
CGATGCTTTACCCAAGATGACCAATAAATTTTTTTTTATTATTATACTTTAAGTTCTAGGGTACATGTGTACAACG
TGCAGGTTTGTTACATATGTATACATGTGCCATGTTGGTCTGCTGCACCTGTTAACTCATTATTTACATTAGGTAT
TTCTCCTAGTACTATCCCTCCCCACTGCCCCCACCCCACGACAGGCCCCGGTGTGTGATGTTCCCCCCTGTGTCCA
AGTGTTCTCATTGTTCAATTCCCACCTATGAGTGAGAACATGCGGTGTTTGGTTTTCTGTCCTTGTGATAGTTTGA
ACAATGAGAACACTTGGACACTTGTGATAGTTTGCTCAGAATGATGGTTTCCAGCTTCATCCACGTCCCTGCAAAG
GACATGAACTCATCCTTTTTATGGCTGCATAGTATTCCATGGTGTATATGTGACACATTTTCTCCCAGCACCTGTT
GTTTCCTGACTTTTTAATGATCGCCATTCTTACTGGTGTGAGATGGGATACCATTGTGGTTTTGATTTGCATTTCT
CTGATGGCCAGTGATGATGAGCATTTTTTCATGTGTCTGTTGGCTGCATAAATGTCTTCTTTGGAGAAGTGTCTGT
TCATATCCTTTGCCCACTTTTTGATGGGGTTGTTTGATTTTTTCTTGTAAATTTGTTTGAGTTCCTTGTAGATTCT
GGATATTAGCCCTTTGTTAGATGGGTAGATTGTAAATATTTTCTCCCATTTTGTAGGTTGCCTGTTCACTGTGATG
GTAGTTTCTTTTGCAGTGCAGAAGCTCTTTAGTTTAATTAGATCCCATTTGTCAATTTTGGTTTTTGATGCCATTG
CTTTTGGTGTTTTAGTCATGAAGTCCTTGCCCATGCCTATGGCCTGAAAGGTATTGCCTAGGTTTTCTTCTAGGGT
TTTTATGGTTTTAGGGATGATCAGTAAAATTAATTATGACATTCTTCAATATAAGAATACTCTGAGGAAATTAGCC
TGTGATCTAATAAAACAAGGAGCAAACAACTGTTTCTAAAAATTTATAACAAATAGTCAAAAAGCCCTCGCTAGAG
ATTGTTTCTCACATTCACTCTATAATTCTGGGTAAAAAAAAAAGCACGTATTTGTTGAAAACCACAGAAGAAGAGA
AAATTGAAAAAGCAGAAAGTGAAAAAAATAAGAATAATAATAAAGATGACAGCAGTTATTTCATTGGAAACAATGC
AAATGAGGAGACAGATGAGCAACATCTTTAGAGTCCTAAAAGGAAAATGCTGTGAAACTAGATTCTATTCCCAGAA
AATAATACCTTTTTCAAATATATATGTATAGTTTTAATTGACAAAGTATAATACATTCATGAGATATTGTTTGATG
TATTGATATATGTGTACCCTATGTAATGATTAAATTAAGCTAACACACCTTGAATATATCTATCAAAAAATAAAGG
GATTTTAGGCCAGGCATGGTGGCTCATGTCTGTCATCCCAACACTTTGGGAGGCCAAGGTAGGTAGATCACGAAGT
CAGGAGTCAAAACCAGCCTGACCAACATGGTGAAACCTCGTCTCTACTAAAAATACAAAAATTAGCTAGGCATGGT
GGCATGCTCCTGTAATTCCCAGCTACTCAGGAGGCTGAGGCAGGAGAATCACTTGAACATGGGAGGCGGAGGTTGC
AGTAGGCCAATATCACACCATTGCACTCCAGCCTGGGCAGTAGAGTGAGACTCTGTCTCAAAAATAAACAAACAAA
CAAACAAACAAACAAACAAATGGATTTTAGACATACAGGGAGTGAAAAAATAACCAGCAGACCTAAACTATAATAA
ATGTTTCAGTCCTATAGGCAAAAAGAAAATTAAGGGACTAGTTGGCACAGCATTTAACGATGAAGAGACAGCTTCA
TCGCCCCCTCCCACTGAGACAGAGAACACAGCCCCAGGGCCTGGGAATTCCTCAACCCAATCCACCACCTTGGGCA
CCTGTGCACTCCTCCTGGGTACCTGAGGTTGGATGCAATCACCTTAGCTATCACCTACCTGCAAACACCACCTATG
GGCATCCCACCCAGCCTATCGCATCTGCTGCTAACAATGCACACTGCTTGGGACCCAGAGAAGCAGCCCACGACTG
CCACTGCCAAGCTGGTTGCCCAGGAGCTGAAGTCCCCAGCACCTACCGAGTCAACCACTGCCACTACTGCCATCCA
AGCAAGCCACCTGGAGGTCCAGGAATCAGCCCACCAGTAACCACCAATACAAATGCCAGTATATACCACCTTGGGG
CACAAAGATAGGAACACTCAGCACACCACTGCCACCACTGGAGCCTGAAGACTGTCTCATGTGGCATCCCAGTCCC
CAGCACATTTTCACCACAGCCTCACTAATAACCACACCCTAATGCACAGAAGAAATCACAGATACCATTAACACTG
TCTACAGCCAAAGAAATTATACAGAGACCATAGTACTCTACTATGTACTACACCCAGAATCAAATCCAAAGTACCC
CAGGCAACCAACACCATAGACTCATCTTCAGGAAAATGCTCTCCACTATGAAACAAAATTCAAAAATAGGAAGAAC
TGACTGTTATATCAGATGCAAAAATATTAATGTTAGGACACAGAAACATGAAAAAGCAAAAAAGAAAAAAAAAGCC
GCAAAGGAAGACAATAGATCTTAATCAAAAAGAAATTTTCAAGATTCTAGATAAAGGACTCAAAACATTTATTTTA
CAGAAGTTCAGTGAGATACAAAAGAATTCTGAAAAAAAAAATACAAAGAAAACATAAAAATAATTCAGGATACAAA
TAAGAAGTTTACAAAAGATATAGATTTAAAAAAAAACAACAGAAATTCTGGAATTAAAGAATACATGACATGAAAT
TCCAAATACATTTGAAAGCTTCAACAATATACTAGGTCAGGCAGAAGGAAGAATTCCAAAACTTGAAGATATACAT
TGTGAAATAATTCAATAAGACAAAAACACACACAAAAAAAGAATGAGCAAAGCCTTTGTGACATTTGGTACAATAT
AAACAACCAAATATTCAAATTATTGGTATCCTCAAGAGCGAAGAGACAAAGAACAGATTAGAAAACCTATTTAATG
AAAGAATAGATGAATAATTCCCAACTCTAGCAAGTGATTTAGACATTCAGATACAAAATATTCAACAATTCTGAGA
AAAATACAATGCAAAAAGTTCCTCTCCATGGCATATAATCAGACTTTCTATAGTATAAGGTAAAATGCAAATCCTA
AAAATAGCAAGAGGAAAGTGTTTAGTCACCTATAAAGGAAACCCCATCAGACTAACAGCAGATTTCTTTTTCTTTT
TTCTTTCTTTTTTTTTTTTTTGAGACGGAGTCTCGCTGTGCTGCCAGGCTGGAGTGCAGTGGCGTGATCTCGGTTC
ACTGCAACCTCTGCCCCCTGGGTTCAGGTGATTCTCCTCCCTCAGCCTCCCAAGTAGCTGGGATGACAGGCACATG
CCACCACACACAGCTAATTTTTTTTTTTTTGTATTTTTAGTAGAGACAGGATTTCACCAAGTTGGCCAGGATGGTC
TCAATCTCTTGACATCATGATCCGCCCACCTCAGCCTCCCAAAGTGCTGGGATTACAGGTGTGAGCCACTGTGCCC
AGCTCTAACAGCAGATTTCTTNGCAGAAATCTTACAGGCCAGAAGAGAATAGGATAGCATAATCAAAGTCCTGAAA
GAAAAGAACTGCCAAGCCAAGAGTAATAAACCAAACATAAATTATTCTTCATAATTGAAAAAGAAATTATCTTTCC
AAGACAAGCAAATGCTGAAAGGCTTCATTACCACTAGACAGGGCCTACAGGAAATGCTCAAGAAAGTCCTAAACCT
GGAATTGAAAGGACAACATTTTCAACATTTCCACATTATCACCATGAGAACATACACAAGTATAAAACTCACTGGT
AAAGCAATCACACAAAAGAGGAAGAGAAAGTTCTCAGATGGTCCTACTACAGAAATCCACCAAAGCATAATGACAA
GCAATAAGAGAAAAAGACAATAACAAAAGACATATAAAACAACCAGAGAACAACAATGTGAAAGGAACAAAGCTGT
TGGGAACAGGACCCCAAAAATCTGGCCATAAACTGGCCCCAAAACTGGCCATAAACAAAATCTCTGCAGCACTGTG
ACATGTTAATGATGGCCATGACGCCCACACTGGAAGGTTGTGGGTTTACCAGAATGAGGGCAAGGAACACCTGGCC
CACTCAGGGCAGAAAACTGCTTCAAGTTGTTCTTCAACCACCAACAATAGCATGAGCGATCTGTGCCTTAAAGACA
TGCTCCTGCTGCAGATAACTAGCCAAACCTATCCTTTTATTTTGGCCCATCCCTTTGTTTCCCATAAGGAATACTT
TTAGTTAATCTATAATCTATAGAAACAATGCTTATCACTGGCTTGAGTTAATAAATACGGGGGTAAATCTCTATTC
GAGGCTGTCAGCTCTGAAGGCTGGGAGACCCCTGATTTCCCACTTCACACCTGTATATTTCTGTGTGTGTGTCTTT
AATTTCTCTAGCGCCGCTGGGTTAGGGTCTCCCCAGCTGAGTTGGTCTCAGCACAAAGCCTCACATATTAATAAAA
ATCTGGAATGTAAACAGATTAAATTCTCCACTTATAAGATGTAGAATGGCTGAATGGGTAGAAAACATGATCTAAC
TACATTCTGCTTACAAAAAAACTCACTGGTAATACCACCTCACCACAATCATAGAGACAGAAGGTAAAAGAATGGC
AACAGGTTATCTATCGAAACCAAAAGTGAGCAGGAGTAGCTATACTTATAGCAGATAAAATAAACTGTAAGTCAAG
AACAATTTTTTTAAAAGATAAGGAAGGCCATCACATAATCATAAAGAGATCCATCCAAGAAGAGAATAGAACAATT
CTAAATATATATGCACTAAACATTGGAGCANCTAGATTCATAAAACAAATATTACTACATCTAACAAGAGAGATAG
ACTCCAATACAATAATAGTGGGGGACTTCAACATCCCAACCTCAGCATAAGACAGATCACCTACACAGAAAATCAA
GAAAGAAACATTGGATTTAAACTGAATTTTACACCAAATAAAGCTAACAGATATTTACAGAGCATTCTGTCTAACA
CTACAGAATATACACTCTTTTCATGAACACATGGAACATTTTCCAGGAGAGAGCCTATGTTAAGCCACAAAACAAG
TCTCAACAAATTTTCAAAAATTGAAATCATATTAAATATCTTCTCTGGCCACAATGGAATAAAACTAGAAATATAC
ACCAAGAGAAATGTTGGCAACAAGACCAATACATGGAAATTAAACAACCTGCTCCTGAATAACCATTGAGTCAACA
GAGAAATTAAGATGTAAATCAAAAAGTTTCTTGAAATAAATGAAAATGGAAACACAACATACCAAAACCTGTGCGA
TACAGCTAAACAGGGCTAAAAGGGAGGTTTATAGCAATAAATACCCACCTAAGAAAAGTAGAAACATTACAAATTA
ACAATATAACAATGAACCTCATGGAACCAGAAAAGCAAGAAAAAAAATATACAAAATTAGCAGAAAAGAAATAATA
AAGATCATAGTAGAAATAAAGACTTTTAAAAACCATGTAAATGATCAACAAAGTGAAAAGTTTGTTCTTCAAAAAC
ATAAACAAAATTGATAAACCACTAGCTAAACAAACCCAGAAAAAAAGAGAGAAAATTTATTAAGAATAAAAGTTCC
TCTTCAAAGTTTTCCTTTTTGGTTTAGAATGATAAGTCTTAAGAAGATAGTTCCTCTTAAAAGCTTCCTTTAAGTT
TCCCTCGCTCTTCATACTAACAAATCTTCTATTTTACTAATGACTCTTTATTGTGCCCCAAAGGAGTTGTTACATA
CAGTAAANAAATAAACACATTCTATGTTCTTGTACTTGAACCAAGGCCTAACTCCTTTGTTCCCTAGTCTGCCTGG
ATCTGCCTGGACACGCCCAGACATGTCACAGCTTGCAGCCTATGCTCTTTCCTTATTTGGAAATATTATTGTCTTC
CTGGTTTCCCATAAGCGACCCCCTCCTTTCCTTGGTTCCCCATTGCGCCTTTACCTTATTTGGGAAAGTTTAAGTT
TTTAGCCAACTGGGATCAGTTTAGATTGTGTGGTCCAGTTCCAGCCAATGGAGACAGGACACAATAGTAGAAACAA
ATTGCGTTAGGAATAAAAACCCCTGCTTTCCTTTGTTCTGGTTGCTCTCGTGGCAATCAGGCTTACAGGTAGCAGC
CTTCTGCAGAAGTAAAAATTGCCTTGCTGAGAAATCCTTTGATCAAGTGTTCATTTCCTTTGTGACTCCAAGCTTT
ATTTCTAACTATTTTATGGGGGCTCTTCCAGGATTCCCATTCTCCTATGGGAAGGGGTCTTTGGTCACCCCATCTA
GGGGAGGTGTACCCCACTGCAGTGGCCCCAGGGACGGGAGATTGAGACTACCCATTGTGATGAATAAATTCAGGCT
CTCAGCAATGTGGAAGGAGAAGGCCCACAAGTACCACGATGACCAGCTAACTGTGCAGAGACCAAGGTAAAAAAAG
TCACAAGGGCAACAGTATTTCCTTGGTGGTCGGGATATTCTGGAGGTTAAAGGGTGTGAAAGGTAACAAGCACTAC
TGCTGTGCAGAGTGAGTGAGTTCAATCTGTGGTTCCGTGGTCACCTCATGTGGCTTATGATGGCCCTCTTGTAGGG
ATCCTGACATTGGGGTTCATACAGTCCTGCTGATGCTAAGGGGAGCCTAATTTCCTCTGGGAAAGCAGCCAGAGTG
GATGAAGCAAAAGGAGAAGATTGCAAGAAACCTCCAGGGTGGGGGTTAAACCTCCAGGGAAGGATAGGCAAGAAAT
CTCTAGTTCGAGGGACTGAGCCTAACCAGTATTCAACATGGGAAATACCCTGAGTAAGACAGAAGGTAAAAAGGAA
AAGGCAAACAGTGAAATCACCTCTGATAGTCCTTTAGGACTCATGTTAAAATATTGGAAGGATACAAGAGGACTAA
ACATAAGAAAAAGCAACAGATGATAAAGTATTATTGTTTCATTTGGACCAAGGAACCTATTCTAAAGCCTTCAGTT
TTCTGGCCAAAATTCGGGTCAAATGAGGCTTGGACTTGCCAACTTTTAATAGAATGTGTAAATGATAAGAGTCCTG
TTTCCCAGGAGGAAATACATTATGCTCTGTGTTGGCAGCAAGGGCCCATCCTCCTTTGTCATCTAAAAACCAAAGG
AGACAAGCCAGAAACTACTTCTCATGAAATTAATACCCCTTACTCCAAGAACCCCACCAACACGTGGGACGCCTTA
GACCATCTTCCCCCAACAAAACTTCTTCCCCCCACCCAGTAGACCAACATCCCCCACCTCCCCCTTCAGTAGAAAT
AACAGTCCCAGACCTTTCCCCTACTCAGACATTTTTTCCCTCCTTTTAACCCTGATTCTTGGGGTCATCCCCAGCT
TGAATGCCCTCCCTCAGGAAGACTTCAACGTGAGTTAGAACAATGTAAAAATGATATCCAAAACTTCCCTTTTCCC
CCTTCCTCTAGGGGTTTGGCTACAAACCTCTTCCCACTGAGGGAAGTGCCTCTAGGAGGAGCAGGCATTGGCTTTT
TAAATGCTCCCCTGACCAGTTCGGAAGTCCAGAGCTTGAAGAGGGAACTCAAGCCACTCTTAGATGATCCCTATGG
AGTGGCAGATCAAATTGATCAATTTTTAGCACCCCAGTTGTATACTGTGGCTGAGTTTATGTCCATCATAGGCATC
CTTTTTTCAGGGGAAGAAAGGAGCATGATGCATAGGGCCGCTATGACCATTTGGGAGTGTGAGCATCCTCCCGGTC
AAAACGTCCCTGCAGCCAAACAGAAATTTCCTGCTCAAGATCCCCAGTGGGATAACAACAACATAGCTCATAGGAG
GAATATGAGAGACCTGCAAGAAATGATAATAAAAGGAATTAGATAATCAGTACCCTGCACCCAGAATATGACCAAA
GCTTTTAATATACAGCAAGGAAAAGAAGAGGGGCCTATGGAATATTTTAATAGGCTTAAGGAACAAATGAGGAAAT
ATGCTGGTTTAGATGGAGGAGACCCACTTGGACAAGGAATGTTAAAAGCTTCACTTTGTCACAAATAGCTGGCCAG
ACGTTACCAAGAAATTACAAAAGATAGAAAATTGAAAGGACTGTCCCATAAAGGAACTCTTAAGGGAGGCCCAAAA
GGTGTATGTACGAAGGGATGAAGAAAAGCAAAAGCACAAGGCAAAGATTATGCTGTTCTTAAAGCAGGGAACTCCC
CAATAGACAGCTCAAAGAAATATCGCCGGTAAATCTTTTAGGTACCCGACTGCCAGACTCTATAATGGAGGTAAAG
GAATCAAACCAGGGAACAAGGGAATAAAAAGAGGAATAGGGCAGAATAAATGCTTCAAATGTGGGAAGGAAGGTCA
CTTTAAAGAGAATGCCCTGAATGGAAAAAAGAAAAAGAAATCACCCACTTATGATCTTTGAGGAGGAATGGGGGGC
CAGGGGCTCTACTCTTTTTACCTCGAGTCCCACCAAGACTCCTTGATAAATTTAGAGGTGGGACCCAGACCTTTTT
AATTGACTCAGGAGCTGCTGATTCCTCAGTTTGTTATCTTCCCCCTGGTGTAACTTGGTCACAAGAAGAACTTTTT
ATTTTGGGAGCAAAAGGGGAGGGGTTTAAAGCAAAAGTCTTAGAAGAAACAAAAATTAAATATCAGAACCACTCAG
TAAATATTAAACTTCTGTTAATCCTGGAGGCAGGAACAAACCTATTAGGAAGAGATTTAATGCTAAAATTAAACCT
AGGCCTTTGTGTTAATCAAGGAAATCTCCTCCCCTCCTTAAATTTGCTTGCTACTCTAAATGAGGGATACATCCAT
CCAGATGTATGGTCAAAGGAAGGAAACTGAGGAAAGTTACAAATTTCCCCAATCCAAGTTAAATTGAAAAACCTGG
GGCAAGAGGTAAAAAGAAAACAATATCCAATTCCCTTAGAAGCTAAGATCAATTTAAAGCCCACAATAGAAAGCCT
TCTCCATGGTGGACTCATTGAACCCTGTATGTCTCCTTATAACACTCCCATACTGCCTGTGAAAAAGCCAGAGGGG
TCATATCGGCTAGTGCAAGATCTTCGATCTATTAATCAAATAGTTCAAACAACTCACTCTGTCGATCCTAATCCTT
ATGCTATTATTAGTAAAATTCCTTACAACCATGAGTGGTTCACAGTAATAGATCTAAAGGATGCCTTCTGGGCTTG
CCCACTAGCAAAGATAGACTTTGAATGGGAGGACCCTCACTCTGGTTGAAAATAACAGTATCGATGGACAGTTTTG
CCCCAGGGATTTACAGAATCTCCAAATTTGTTTGGTCAAATACTAGAACAGATCATAGAAAAGTTTTACAAACCAC
CATATATATGCCTGCTCCAATACATGGATAATATCCTTATTTCAGGGGAGGATAGACAAAAAGTAGAAGGATTTTC
AATAGGTTTTTTAAAAATAATTTGCAACTGGAGGGGTTATGAGTGTCAAAAAAGCAAACTTCGGTTTGTAGAACCT
GAAGTCAAGTATTTAGGGGACTTAATCAGTAAGGGCAAATGGAGAATTGGATTTGAATGGATTGAAGGCATCATAT
CCTTCATATGATATCCTGAGACAAAGAAGGAACTTAGAAAGTTTTTAGGGTTAGTAGGGTATTGTTGCTTATAGAG
AGACTCTTATGCTTTAGTAACCAAACCTCTACATAAAAAAAACTTACACAGGATGAATTGGACCCCCTTATATGGT
TTTACAAGAAATACAGCAGATAAAAAAATTAAAAGAGTTACTAGTGACAGCCCATGTTCTAGCCTTACCCTCTTTA
GAATTGCCCTTTCATCTTTTTGTTAATGTAGGTAATGAAGTAGCCTTAGGGGTACTTACTCAAACACACGGAGGCC
ATTGGCAACCCATAGCCTTCTTGTCAAAAATTCTTCACCCAGTAGCCCATGGATGGCCCGAGTGTGTCCCATCAGT
GGCAGCAACAGCCTTGCTCATAGAGGAGAGCAGAAAATTAACCTTTGGGGAAATCTTATCATTAGTACTCCTCAAC
AAGTGAGAACAATTCTCAATCAGAAGGCAGGAAGGTGGCTTATTGATTCAAGAATTTTAAAGTATGAAGCCATCTT
GTTAGAAAGAGATGATCTAATCTTAACCACTGATGACTCAATTAATCCAGCTGCTTTCTTAACAGGAAACCCCAAC
CCACAGACCCCTGATCTTTGCCCAGAGCACAGATGTTTAGATTTAATTAGTTATCAAACAGAAGTTAGACCTGATC
TAAATGAAACACCCTTTCATACTGGGAAACACCTGTTTGTAGATGGTTCCTCCCGAGTAATCAAAGGAAAAAGGCA
TAACGAGTATTCAACGATAGATGGAGATACCCTCACAGAAATAGAGTCTGAAAGGTTACCAAATGACTGGTCTGCA
CAAACATGTGAGTTGTTCACATTAAATCAGGCCTTAAAATTTCTGCAAATCAAGAAGCAACAATCTATACAGACTC
CAGGTATGCCTTTGGAGTAGTCCATACTTTTGAAAAAATTTGGACTGAGCGGGGCCTCATTAACAGCAAAGGTCAA
ATTTTAGACCATAGGGACCTAATAATACAAGTACTAGAAAATTTACAATTGCCAAAAGAAATAGCTGTTGTACATG
TCCCACGACATCAAAGGAATCCTTTATTTGAAGGCCAGGGAAATAATCTCACTGACCAAATAGCCAAGCAAGCTGC
CTCTTCTCCAGCAGAACCCATTTTTCGACTAACCCCTTGTCTTCCATCTTCAGCTGCACTCCCTATCTTCTCTCAA
GCAGATCAGGAAAAACTGAAAAAAATAGGAGCTGAAGAAAACTCAAAAGGAAAGTGGGTATTACCAGATGGAAGGG
AAATGTTACCCAAACTTTATGAGGGAAATATTGTCACAGCTTCATCAAGGATGTCACTGGGGTCCTCAAACCATGT
GTGATGCAGTCCTTAGGGTTTATGGGTGCATAAGAATATACACCCTCGCCAAGCAAATGGTGGAAAGTTATATACT
GTGCAGAAAATCTAATAAGCAGACCCTCAAAAGACAACCTCCCGGAGGGAGAAATCCTGGGTTAAGGCGATTTCAA
AGTGTCCAAGTTGACTCTACTGAAATGCCCCCAGTAGGCCGCCTTAAGTATTTACCGGTAATAGTAGATCATCTTA
CCCACTGGGTAGAAGCCATCCCCTTTCCAAGTTCAACAGCCAGTAATGTGGTCAAAGCTCTGTTAGAACATATCAT
ACCCAGGTTTGGAATAATAGAGAACATTGATTCAGATAATGGGACCCAATTTACCGTGTACATTATTAAAAGACTA
ATCCAAGTATTAGGGATAAAATGGGAATATCATACTTCCTGGCCTCCACCTTCATCTGGGAGAGTAGAAAGAATGA
ATCAAACTTTAAAGAGTCATCTAACCAAATTAATTTTAGAAACTCGCCTACCATGGACAAAATCTCTTCCCATTGC
TTTACTAAGAATCCAAACTTCTCCTCGGAGGGATCTTGGCTTGTCTCCTTATGAAATGCTTTATGGGTTACCTTAT
CTAAACACTACTACTGACCTTCCTATGATCGAAACAAAAGATCAGTTCCTTAGAAATTATGTATTTGGTCTGTCTT
CCACTCTTTCGTCCCTCAGGACTCAAGGTCTGGTAGCACAGACTCCACCCCTTGAATTCTGGGCCCACCAACATCA
ACCCGGAGATCACACGTCCTTATCAAAGGCTGAAAAGAGGGAAAGCTTGAGCCTACCTGGGAAGGACCCTATCTGG
TGCTCACAACAACTGAAACAGTAGTTCGGACCACTGAGAAAGGGTGGACCCATTATACTCGAGTCAAGTAGGCGTC
ACCTTCTCCAGAGACATGGACCATTATTCCGTCAACTCCCACCAGAGTAACGATAAAAAGAAAAACTTAATCTATC
TCTTTTTCTTCTTTTCTTTCCCTTAACTGCCCCCATCTCATCATTAATGTAACTAGATCAAGTTTGCCCAAAATTA
TTACCTTTGATGCTTCTCTAGTCATGCCTTGTGGAAATTTACAGGACCAAAGACAGCTTACTGCCTCAGATAAATA
TCTTTGTCCCTCTTGAACTTCCTCAGATTGGAAAGATATAACAAATTCTTGTAAAGATCATGATAACCCCAAAAAT
AGTAGCTGTATGAACTGGTGGGAATGGGACTCTTGTCCCCTTAAAACAGAGTCCCTATGCTATACTTGGTCTAATG
TCCTATGGAACACCAAAGGTCCAGGCTGGACTGCTCCCACCAGTCTTTGCCAGTCCTTAAAGCCATATATCCATTT
TACAAAAGGGATTGCCTCTTCTAATTGTCCATATAACCAGTGTAATCCTATTCGGCTTACTATTACTATTGCAACA
TCCCAAAACTCTTTCCCTTCATTAAGTCATATTTATAGCATAGGGGCCAACATCTTGGGAAAGGATCCTAGGGGAA
TTTTTGAAATCTGCTTTATTGCCCCTTCCACCTCTCTAACACATCTTCCTCAAACCATCACCCCACCTATACCCAG
TGACAAAACCAAAGTAGCTATAGTAGAAGTAAAAGACCTGAGGTAAACTCTAGCCACTGAAACAGGGTCTCAGGAT
GCAAATGCCTGGCTGGAATGGATTAAATATTCCATCCACACTCAAAATAAGAGCGATTGTTATGCTTGGGCACAGG
GCAGACCAGAGGCCCAAACTGTCCCCTTTCCACTCAGATGGTCTTGTAATAGACCAGATATGGACTGCATGGTAGC
TCTGCTCCAAGACCTCACAGCCTGGGGTAATAAATCGTGTCAAAGACTTTCTTTGCTGTTCCCTGAGATCAGCCCC
CAAAGGCCATTCAGCCTCCATCTTCTGGGACCAAATTTGCTTTGTGTCTCTCACAACAGGGAGAAAACTTGGCTTT
CCTTGGAAACCTGACAGGATGCAGTGAGGTCAAGCACTTCCAAGAGCTAACCCATCAGTCCTCCCTTATCCATCCC
CGAGCAGATGTATGGTGGTATTGTGGTGGACCTTTGTGGGAGACTCTGCCAAATAACTGGAGTGGTACTTGCACTC
TAATTCAATTGGCTATCTCTTTCACCCTGACATTTCATTCACAAAAATATTAACAACTAGGCATCGTAGTACAAGA
ACTGTTCCTGTGAGTCTTTTGATCCCAACGTTTATATAGATGCTATTGGAGTACCAGATGAATTTAAAGCTTGAAA
CCAAATAGCTGCAGAATTTAAGTCATTATTCAGGTGGGTGACTATCAATAAAAATATAGATTGGATAGCCAGGCGC
AGTGGCTCACGTCTGTAATCGCAGCACTTTGGGAGGCCAAGGCGGGCAGATCACGAGGTCAGGAGTTTGAGACCAG
CCTGACCAACATAGTGAAACCCTGTTTCTACTAAAAATACAAAAATTAGCTGAGTGTGGTGGCACGCACCTGTAAT
CCCAGCTACTCAGGAGAGTCACTTCAACCTGGGAGTTGGAGGTTGCAGTGAGCCGAGATCACACCACTGCACTCCA
GCCTGGGCGACAGACTGAGACTCCATCTCGAAAAAAAAAATGTAGGTTGGGTAAATTATATTTACTACAATCAGCA
ATGATTCATTAATTGCACAAGGGATGCTATGAGGGAATAGCTGAGCAACTAGGACCCACCAGTCAGATGACTTGGG
AAAATAGAACAGCACTAGACATGATACTAGCAGAAAAAGGCAAGGTTTGTGTTATGATTGGAACTCAATGTTGTAC
TTTTATCCTTAATAATACTGCCCCTGATCGAACTATAATAAAAGCACTACAAGGTCTTACTGCTTCATCAAATGAA
CTAGCTAAAAACTCTGGAATAAATGATCCTTTCACTAGCTTAATGGAAAGGTGGTTTGCTAAGTGGAAAGGACTCA
TGTCCTCAATTCTCACCTCACTGGCTCTCATAATCAGTGTACTTATCCTTGTAGAATGTTGTATTATATCTTGCAT
TCATGGATTAGTGCAAAGGCTTATAGAAAAGGCTCTTACCAAAACCCCTTTCAATTCTCCTCACCCTTACTCAGAT
AAGTTGTTCTTTCTAACTGATTAAGAAGAGCAACAAAATCAAGATATGTTAAGGAGATTTGAAGAGGAAGAACTGT
AAAATCAAGAGGGGGAAACTGTAAAGAATAAAAGTACCTCTCCAAAGTTTTCCTTTTTGGTTAAGAATGATAAGTG
TTAAGAAGATAGTTCCTCTTAAAATCTTTCTTTAAGTTTCCCTTACTCTTCATACTAACAAATCTTCTAATTTACT
AATGATTCTTTATTGTGCTCCAAGTAGTTGTTACATACAGTAAAGAAATAAACACATTCTATGTTCTTGTACTTGA
ACCAAGGCCTAACTCCTAGTCTGCCTGGATCTGACCAGACATGCCCAGACATGTCCCAGCTTGCAGCCTATGCTCT
TGCCTTATTTGGAAATCTTATTGTCTTCCTGGTTTCCCATAAGTGACCCCCTCCTTTCCTTTGTTCCCCATTGCAC
CTTTACCTTATTTAGGAAAGTTAAAGTTTTTAGCCAACCGGGATCAGTTTAGACTGTGCGGTCCAGCTCCAGCCAA
TGGACACAGGACACAATAGTAGAAACAACTTGCGTTAGGAATAAAAACCCCTGCTTTCCCTTGTTCTGGGTGCTCT
CGTGGCAATCAGGCTTACAGGTAGCACCCTTCTGCAGAAGTAAAAATTGCCTTGCTGAGAAATACTTTGTTCAAGT
GCTCATTTTCTTTGTGACTTCGAGCTTTATTTCTAACAGACTCAAACAAAATCAGAAAAGAAAAGAGAAACATTAC
AACTGATATTGCAAAAGTACAAAAGACCATCAGAGACTATTATGAACAATTGTGTACTGACAAACTGGAAAACCTG
GAGGAAATGGATAAATTCCTGGAAACAAACAACCTGCCAAGATTACATTAGGAAGAATAGAAAACCTGAAGCCCTA
ACATACATGAACCATTCTCACAGTGGTCCCAGTGGGCCCCTGACCTCTCTGTGATTATTTCCTCAGTGCTTGTTTG
CATAATTGAAATAGACCTACTCAGCAACAGCCAAAATCCACAGATCAGTTCCTTTTTTTAACTTTTATTTTCAGTT
CAGGGGTACAAGTGCAGGCTGTTTTACAGGTAAACTTGTGTTGTGGGAGTTTGGTACACAGATTATTTAATCACCC
AGGTTTTAAGCCTATTACCCATTAGTTATTTTCCCTGATCCTCTTCTTCCTCCCACCCTCCACCTTCTGAAAGGCC
CCAGTGCCCAGTGTGTGCTATTCCCCTCTATGTCTCTATGCATTCTCATCATTTAGCTCCCACATATAAGTGAGAA
CATGCAGTATTTGGTTTTCTGTTTCTGCATTAGTTTGCTAAGGATAATGGCTTCCAGCTCCACCCATGTCCCTGCA
AAGGACATGATCACATTCCTTTTTCTGGCTGCATAGTGTTCCATGGTATACATGTATCACGTTTTCTTTATCCAGT
CTATAATTGGTGGGCATTTAAGTTGATTCCATGTCTTTGCTATTGTGAATAGTGCTGCAATGAACATATGTGTGCC
TATGTCTTTATAATAGAATAATTTATATTCCTTTGGGTAGAATGGTATTTCTCTCTTTAGGTCTTCAAGAAATCAC
CACACTGTCTTCCACAATGGCTGACCTAATTTGCACTTCCACCAAGAGTATATAAGTATTCCTTTTTCTCCACAAC
TTTGCCAGCATCTGTTATTTTTTGACTTTTTAATAATAGCCATTCTGACTGGTGTGAAATGGCATCTCACTGTGGT
TTAGATTTGCATTTCTCTAATCATCAGTGATGTTGAGCATTTTTTCATGTTTGTTGGCTGCATGTATGCCTTCTTT
TGAGAAGTGTCTGTTTATATGCTTTGCCCACTTTTTAATGGGGTTGTTTTGTTTTTTCTTGTAAATTTGTTTAAAT
TCCTTATAGATATTAGACTTTTGTCAGATGCATAGTTTGCAAAAAATTTTCCCCCATTCTGTAGGTTGTTTACTCT
GTCGATAGTTTCTTTTGCTGTGCAGAGCTCTTTAGTCGAATTAGATCCCATTTGACAATTTTTGCTTGTGTTGCAC
TTGCTTTTGGCATCTTTGTCATGAAATCGTTGCCCATACCTATGTCCTAAATGGTATTGCCTAGGTTCTGTTACGG
TTTTTACAGTTTTGGATCTTACATTTAAGTCTTTAATCCATTTTGAGGTGATTTTGGTATATGGTGTAAGAAAGGG
GTTCCATTTTAATCTTCTGCATATGGCTAGCCAGTTATCCCAGCACAATTTATGAATAGGGAATCCTTTCCCCATT
GCTTGTTTTTGTCAGGTTTGTCAATGATCATAGTTTGTGTGGTCTTATTTCTGGGTTCTCTATTCTGTTCCATTGG
TCTATATGTCTATTTTTGTACCAGTACCATGCTGTTTCGGTTACTGTAGCCTTGAAGTATAGTTGAAATCAGGTAG
CATACACTGGACGTGGTGGCTCATGCCTGTAATCCCAGCACTTTGGGAGGCAGAGGCAGGTGGATCATGAGGTCAG
GAGTTCGAGACCAGTCTGGTCAACAAGGTGAAACCTTGTCTCTACTAAAGATACAAAAAATTAGCTGAGCGTGGTG
GCATGTGCCTGTAATCCCAGCTACTCAGGAGGCTGAGAAAAGGAGAATCACTTGAACCCAGGAGGCAGAGGTTGCA
GTGAGCCAGGATTACACAATTGCACCCCAGCCTGGATGACAGGGTGAGACTCCATCTCAAAAAAAAAAAAAAAAAA
TCAGGTAGTGTAATGCCTCCAGCTTTGTTCCTTTTGTTTAGGATTGCCTTGGCTATTCAGGCTCTTTTTTGGTTCC
AGGTGAATTTTAAAATAGTTTTTTCTAGTTCTGTGAAGAATGTCAGTGGTAGTGGCCGGGCACGGTGGCTCACGCC
TGTAATCCCAGCACTTTGGGAGGCTGAGGCAGTGGATCATGAGGTCAGGAGATGGAGACCATCCTGGCTAACGGGG
TGAAACCCCATCTTTACTAAAAGTACAAAAACTTAGCCCAGCATGGTGGCGGGTACCTATAGTCCCAGCTACTCGG
GAGGCTGAGGTAGGAGAATAAAAAAAACCTCCTGAATTCGTTGATCTTTTGAGTGGTTTTTCATGTCTCTGTCTCC
TTCAGGAGACAATACCTTTGTTAATTTTTTATTATTACGTCTTGTCTTCTGCTAGCTTAGGGATATGTTTGCTCTT
TGTTCTCAAGTTCTTTTGGTTGTGATTTTAGGTTGTTAACTTGAAATCTTTCTAACTTTTTGATGTGGGCATTTAA
TGCTATAAATTTCTCTCTTAACACTGCCTTAGCTCTTTCCCAGAGATTCTGGTACATTGTATCTTTGTTCTAATTA
TCTTCAAAGAACTTCTTGATTTGTGCCTTAATTTCATTATTTACCCAAAAGTCGTTCAAGAACAGGTTATTCAATT
TCTATGTAATTGTATGGTTTGAGTGAATTTCTTAGTCTTTATTTTGAATTTGATTGCACTGTGGTCCAAGTGACTA
TTTGTTATGATTTCAGTTCTTTTGCATTTGCTGAGGAGTGTTTTACTTCCAATTGTGTGATTGATTTTAGAGTAAG
TGCCATGTGACAATGCAAAAAATGTATATTCCATTGCTTTGGGGTGAAGACTTCTGTAGATATTTATCAGGTCCAT
TTGATGCAGGGCTGAGTTCAGGTCCTGAATATCTTCGTTAATTTTCTGTCTTGATAATCTGTCTAATATTGTCAGT
GGAGTATTAAAATCTCTTACTATTATTGTGTGAGATTCTAAGTCTCTTTGAAGGTCTCTAGGAACCTACTTTATGA
ATGTGGGTGTTCCTGTATAGGATGCATATATATTTCAGATAGTTAGATCTCCTTGTTGAATTGAACCCTTTACCAT
TATGTAATGCCCTTCTTTGTCTTTTTTTTATCTTTTTTGGTTTAACACCTGTTTTGTCAGAAACAAGAGTTGCAAC
CCTGGCTTTTTTTCTGCTTTCCATTTGCTTGGTAAATTTTCTTCCATCTTTTTGTTTTGAGCCTATGTGTGTCATT
GCATGTGAAATGGGTGTATTGAAGACAGCATACTGATAGGTCTTGGTTCTTTATCCAGCTTGCCACTCTATGTCTT
TTAAATGGGGCATTTAGCCCATTTACATTTAAGGTTAGTATTGATATGTATGGATTTGATCCTATCATCATGATGT
TAGCTACTTACTTTGCAGACTTTCTTATGTGGTTGCTTTATTGTGTCACTAGTCTGTGTACTTCATTGTGTTTTTG
TAGTGGCTGGTAACTGTCTTTCCATGTTTAGTGCTTCCTTCAGGAGTTCTTGTAATGCAGGTCTGGTAGTAATGAA
TTCCCTTGGCATTTGTTTGCCTGAAAAGAATCTGATTTCTCCTTTGCTTATGAAGCTTAGTTTGTTTGGATATGAA
ATTCTGGGTTGGAAACTCTTTTCTTTGAGAATGTTGAATACTGGACCTCAGTCTCTTCTGGCTTGTAGGATTTCCA
CTGAGACATCCACTGTTAGTCTGATGGGCTTCCCTTTGTAGGTGATCTGGCCTTTCTCTCTGGATACCCTTGACAT
TTTTTCTTTCATTTTAACCTTGGAGCATCTGATGATTATTTGTCTTGGGGATGATCTTCTCATGGAGCAGCTTACT
GGGGTCCTCTGCATTTCTGAATTTGAATGTTGGCCTGTCTAGCTAGGTTGGGGAAGTTCTCATGGATCATATCCTG
AAATGTTTTCCAAATTGGTTCCATTCTCCCCATCTCTTTCAGGTACACCAATCATTCATACATTTGTTTCTTTACA
TAATCCCATGTTTTTCAGATGTTTTGTTCATTCCTTTTCATTCTTTTTTCTCTAGTCTTGTCTTCTTGTCTTATTT
CAGAAAGCCAGTCTTCAAGCACTGAGATTCTTTTCTCCACTTGCTCTATTCTGCTATTAATACTTGTGAGTGCACC
ACGAAATTCTTATAAAGTGTTTTTTACCTCTATCAGGTTGGTTATCAGATTCTTTTGCTGATTCCTTCTCATCTCT
ATGGGCTTATCTACCCTCAATCTTTGAGGTTGCTGACCTTTGAGTGGATTTTTTTTCTCTTCTTTTGTTTTCTCTT
TTAACAGTCTGGCCACTTTTCTATAGGGCTGTACAGTTTGCTAGGGGCCTGCTACAGTCCCTAGTTGCCTCAGATT
TTCCAGTACCTGAAGGTATCACCAGTGAAGGCTATGTAACAGCAAAGATGGCAGTCTGCCCCTTCCTCTGGGAGGT
CTGTTCCAGGAAAGTACAGACTTGTTGCCAGCCTGAACACACCTGTAGGAGGTGCTGGAGACCCTGGTTGGGAGGT
CTTGCCCAGTCAGGAGGAACAGGATCAGGATCTAGAGGTTTACTAAAGGAGGAATGCTCCCACAGGAGACACAGAA
ATGAACCATTGAGCTGGAAGCTAAGACAGCTACCCAGGCCCTTCAGAGCCTCATGATTCTGAATCAACAAAGGAAG
GAGTTACTGCACTGTCTGGGGTGATTGGTCCTGGCCATCAGGGGGAATTGGTCTACTACTACACAATGGCAGTAAA
TAGGACTGTGTCTGGAACACAGGAGATCTTTTAGAGCATCTCTTAGTAATACTCTGCCGTATAATTAAAGTCAATA
AAAACGGCAACAACCCAAGTCAGGTAGGACTAGTAATGACACATACCCTTCAGGAATGAAGGTTTGAGTCACCCAA
GCAGGAAAAGAAACCTGATCAGCTGACGGACTTGCTGAAAGAAATGGGAATATGGGATGGTTAGTGAAAAACAACA
AAAAAAGTATTTATAAATACTAGTTGTGGCCGGCACAGTGGCTTGTGCCTGTAATCCCAGCACTTTGGGAGGCGGA
GGCAGGTGGATTGCTTAAGTCTAGGAATGTGAGACCAACCTGAGCAAAATGCTGAAACTCCATCTCTACTGAAAAT
AAATACAAAAAATTAGTCAGACTTGGTGGTGTGCTTGTAGTCCCAGCTACTTAGGAGGTTGAGGTAGGAGGATCAC
TTGTGCCCAGGAGGTGGAGGTTGTAGTGAGCCAAGGTAATGCCACTGCACTCCAGCCTGGGCAACAGAACAAGACC
CTGTCTCAAATAAATAAGTAANTAANTAAGTAAATAAATAAATAAATAACCAGCTGTAACCATGTGAGCACTTACA
GAAATAAAGACAGTAAATGTTATCAGCATTACTTCTTTATGTTTTTATAATGATATTTATGTCTGTGTGTATGTAT
CCTTACATGTATTGAGCAAATACCTTTCTTTTCATCTTTTCCTGATGATCTGTAATAAAACATAAGATGTGCTAAC
AATAATTCCCTTTTATTAATAAAAGTTAACTTTTATTACGCATGGTATTTAGGTTACAGTATATCAAAGAGAAGAG
TGCATATCATGCATCTTTTTCTGTGGAAAGGGTTAGTGTGTTTCTAGTTGGATGCAAGTTAATTGTATCATCTTTG
GCAAAAGTAAGACTTTATAATTGTTTTTATTTGGAGACGAAGTAAGGTTTAAGGAAAATGTTAATGGGTGCCAAGT
TCACAAGCATGGACTGGGATGGTGGGTCTTATGTGTCAATTTTACTAAACCATAGCACCTAGTTATTTAATCAAAC
ACTAAATTTATATGTTGATATGATGGTGTTTTGCAGTTGTGATTAAAATCTGATATCAGTTGATTTCATGAAAAGG
AAATTACCCTAAAACATGTGGGTGGGACTTTTTGAATCACTTGAAGGCCTTTGGGCGAAATGTGAGTCTTCCCAGA
GAAGAAAAAATTCTGCCTCAAGATGACAGTAGCAACTACACTTTAGGTTTCCAATCTGCCAGCCTGGTCTACAAAT
TTTAGATTGAAGGCTGCAACATTATGACTTTCCAGCCTTCCAGCCTGCCGAGGTGTAACTTGTGACATCAATAACG
TAAAATGAGGGGTGGGAAAAGATGTAAAGAAGCAGAGTTTCATATTGATTTCAGTTCAAGATTGTTATAACTGTAT
TGTTATATATAATACCACAGTAATCACAAAGAAATATACATAAAATATACACAAAAGAAAATGAGAAGGCAGTTAA
AACATATCACTAGAAAAAAATCGACTAAATGAAAAATGACTAAAAACAAAAGAAGGTTGGAATGAAGGAGATGAAG
GACAAAACAGCTATAAGACATGCATAAAACCAACATCACAATTACAGAAGTAAATCCTTCCTTATCAGTAATTACT
TTAAATGTAAATGTAAATGGATTAAACTTCCCAATCAGAATTCAGAGATTATCACAATGGACAGAATAAAAAGGAT
CTGGCTGGGCACGGTGGCTCACACTTTTAATCTCAGCACTTTGGGAGACGGTGGCTGGCAGATCACAAGGTCAGGA
GTTCGAGACCAGCCTGGCCAACATGATGAAACCCCGTCTCTACTAAAAATAGAAAAAAATTAGCCGGGTGTAGTGG
CAGGTGCCTGTAATCTCAGTTACAAGGGAGGCTAAGGCAGGAGAATTGCTGGAACCGGGGAGGCAGAGGTTTCAGT
GAGCTAAGATCGTACCACTGCACTCCAGCCTGGGCAACAGAGTGAGACTCCGTCTCAAAAAAAAAAAAAAAAGATT
CTCACTTTGGATCTAACGACACACAGAAGTTGAAATTGGAAAGACAGAGAAGGATATTCACAAAAATAGTAACTAA
GAATTTTTAATGATCTCTTATAGGGTTCTATGTGTTGGCTGGGCTCAGATGATGGCCTGGGAGTGTTACATGGTCC
AGGGATTGAAGTGGAAGGAACCAGACGCAGGCAGGATGGTTAACAGCAGTGCCCAGACTGACACAGCCACACTTCT
GCCATATTTTATTGGGTAACGCAGATATCAGATCTGCTTATGTTTAAGGAGTGGAGAGAACTTACCTCTTGATGAG
GGAGTGGTGGGTCAAACTAAAGAAAAGCAAGTGCGATGGAAGATATTTAGATATTTTTGCAGCCCTCTTTGGAAAA
TACATGCAATTCAAAACATCCTATCTGCCAGAAAGGAAGAACACCATTACGCCTTACCCCTCCAGTTTAAGCCCTG
TGATTCTATAGTATCCTGGGGAAGGATCATGTCTTAGGAAGCAGGTGGGGAGTCGAAGCTCTAAATAAAGCCCCAT
CTACACCCACAATTTCACTTTATTTTGATGAAATTTTGTGTCATTTTTGATGAAATGGGGGCTCACAAATCTCTCT
TCAGGGATCTCTCCAGTGGCGGGGACGATATGCTCCCTTCTCCACAGTTCTACTTTATAGATAAGTGGAACTAGAT
CTCAGTTTCAGTTATTTAAAACACCAGAGACTTGGATGCATGCATGTCACAAAAAGGTGAAAGAATGCTCTCTGTC
ACTTTCCCTTCTCTCACTCACTGCGCCCTGAACAAGAACCTGCTCTACCCTGACCTTTTGAATGAGGAGGAGGGTC
GCACTCCTGCCTAACCTTCAACTTTCCAGAGATAAACCTCCTGCCCAAACAGCATGGTTCATTATAAAGACCAAAC
CCTACACCAAGAGCTCTCCCCTTATCCACCATGGCAGAAACTGAATCCTATGGAAAGGCCCAGAAAATTCTGGCTC
GAAATGACCAGCGTCATGTCCCTTCCACCAAAATAAAGCCAACATATTCATCACAACATGGGGTATTGATAAGGAT
ATCAAAATGATTACCAAAAAAATTTGACAAATATGTTCTTTTTATTACATCAACTTTTGTTTCCCTCCAAATATAA
GAATCACTAGAATGGAACAAGGGTTTCTGGTATGACACAGCACCAGGTCTTGGGGCAGCCAGAGTTAGGGAGGGCT
CACCCTCTGGGTGACACCAACCTTTGGTTCTCCATTTTTTTGTTCTCATTGCAAGAACAAAATTTCAGAGTATCAG
AGCCAAAGAAATTTACTCTATTTTATTCTTTAGAAAATGCAATCTCAAGGATAATCAGACTGTTATGGGCTAAATT
GTATCCCCCCAAAATTGGCATGTTGATGTCCTAACCCCCAGTACCTGAGAGAGTGACTGCATTTGGAGAGGTTTTA
AGGAGGTCATTAAGGTAAAATGAGGTCATGTGAGTGTGCCCTAATTCAATGTGACTAGTGTCTTCATAAAAAGAGA
AGATTAGACACAGACAGATACGGAGGAATGACCATGTGAAGACAGAGAGAGAACACATCTACAAGCCAAGGAAAGA
ACCTTAGAAGAAATCAGCACTGCCAACACCATGATCTTGAACTTCCAGCCTCCAGAACTGTGAGACAATGAATTTC
TGTTGTTTAAGTCACCCAGTGTGTGGTACTTTGTTATGGCAGCTCTAGCAAATTAAAACAAACATATTATATTGCC
CAATGGAGAGTTTTACCTGACTCCACTCTGGCTCCAATTTGATTAAGGTCTATATGCTCTGGGACCTCTCCAGCTC
CTCATGAGAAATGACAAAGAATGCCATGTCCAGCTCCTGGGGTCCCTGATGGCAGTGAGAGGCAGCTCCTAATCGA
GGGAGCTTTGGGGTGACGAAGGCCTCATAGACTTCATCACTGTTGGGCCACTTGGCTTCAGTCCCCTGACCACAAT
GGATCCATGGGCCTGGATTCACCTCTGACATTCGTGTCTTTTTTTTCCACAAGTGGTCTCTGGTAGAGATGGAGGG
AAAGGACACAGGGATTCAAAATCCTGACTATCCTAGTGTCCTCCCCTCACTTCCACTCAGAGACAACACGGCCATT
TCTCTAAACTCCTGAGGACGTGGCCAACATAAGTAATGGAGCACGGGGAGGTGCCTTGAACAGAGCTGCATGGAAG
GGTGGCTGGTGGCCTCTAGACCCTTTGTCTATACGTGCTGGGCTTCTTTAGTATGAAATGTCCCAGAGACAGCCAC
ATTCTATGCTTGGATCCTCAAACCAAAATGTGTGAGGGTCTTAGCCCTGGCCAACTGAGCACTGAATTCAAGAGAG
ACCAGGAAACCTTCTGAATATTGGGACACTTTATTCCTGAATCCTGAATCCTGGAAGTTGGCTTCTGGCCAGGGAG
GCAGGTGTCATTATATCTGGGCTGCCTCTGTGAGGCCTCTTTTCCCTCTGCCTTTCACATCTTTCTACCCCTGTTA
AAATCCCAGAACAGATAGAAACATTCTGCCTTCTTAGATGCCTCTTGCTACAAATTTCAATGCCATTTGATTGAAT
TAATAAATGAAAGCAACTTTAATACAAGATGAGGTTTAAAGAATGGTATTTGACATGGGTTTAGAATGAATAAATG
TAAATGATTCCAAATTAATACAAAAAAGTGCAATCACAAATGTATGAATGCCCAGAAGTGTGAATGAGGGGCAATG
CACACAAGAAAAAAGGTGTGCACAGGAAGAAATTCTTCATTCCACGTTTTTACTGATCCCTCTGGTCAAGATGGGC
TAAAAGTCCAAAAATCTGCTTTCCTCACATGGTCAAATGATTCAGCATAAAATACAACTGACTGAACACAATTATC
AATGCTTTAAATATATTAGAAAAAATCAATAAGGAAAAATAATCCTACCATCACTAAGATTTTAAGTTATAATAAA
ATAAAGCCTAGAAACGTATCGTTTGTTCCCAATTCCAATGATTATAGGATTGCAGTGTTTGGCTTTCAAAAGACCA
TTTAAAACAACTCAGGGAAGTTTTCTTAACAAACTCTATAGCAGGACCACAGCTAAACCTATGAGTAGTAGTAGAA
GTAAACAATGACACTGTCTTTAAAATTTCATTTTGAAAAACATGTTTGAAAGTGGCTGACTGGCTCCTAATTTAAC
CACTACCATTTGAAGTCATATGTTAGATTGGCAAAGTCGTCGTAATTTTTATGTATTTTATTTTTGTAATGCAAGT
GCTTCACTCTGCTATCTGATAAAAGAAAATACCAAGCCAGGCATGGTGATGCATGCCTTGTAGTCCCAGCTACTCA
AGAGCCTGAGGCAGGAGGTTCCTTCAGCCCAGGAGTTCAAGGCTGCAGCAAGCTATGATGGTGCCACTGCACTCCA
GCCTGGGCAACAGATAATGCAATTTTAATGTAAAATTTTAGTGTTAGGAGTTCAACCTTGAACTGATTCTGTGAGA
TCCTATTTGAGAGAAGCTGTTGTGTACAATGAAGTTTGGGGAATATCAACAAGAAGTAAATGCCTACCTTGTTTCT
AATTTACAAACTAAAAGGAAATTCCATAAACTTTTAATGCTAAGCTAAATCATTGTAGAATGAATGACAAAAAAAT
TATCCTACAAAACATAACTAGTTGCTAGATATATTTGAACATTAATTAGAAAAAAGATGAATGAAAGCCATGTTTT
AGGAATAAAAACTACAAAAGAACTGTAAAAGCTTATCATGCCCTTGGCTGGAGTTACGCAAAGTTACTACTCACCC
GTAAATTGGGATTAGCATGGTGGCTACTTCTTAAAGTTGATACAAGGATTAAATGTGTTTTAATGCATCAAAAGCA
TCTAGAAAAGTGCCTGAGACATAGTAAGTGCTAAAGTCTCTCTAAGTAAATAAAAAATATATAAAAGTGATGCTAA
ATTCACGATTCTGATCCTCTGTGCACACCCAATGTGTTTAAAATTCTGTTCATCTTCCCTTCCACATTCACAGTGA
AATGTTTCTGATTAATTTAATGGGCTGTGTGAGGTGTTCATTATCTTTTACATTAAACTTGGTACATTGTACAACA
CTCTTATATTGTGATAATTTACCAAGCTAACACAGGCTTACCTTGTTTTTAATCCTTTTCACACACAGCATAGAAT
TATATTGCTATATTTTTAAATTTCAGATGATTTGAAAAATATTTACACTATAGTAGCCAAACAGTAATAATTATAA
GCAACTTGGATAAAACATTTGAAATTAGAGAACTATATTTAGAATAAATTTCTCACCTTACAGAAAAATTTCCCAT
TGAAAACCCCTTTATTTCGCATACAAATAAGAATTCAGCATGTTTTTCTATACCCAGAGAGAACAGAGATAAACAG
GGTCCTTTCGTGTGGTTTGGGTATTTTATTAGATGCTGAGTTCAAGAAAGTCTCACCCCTGAAGAAGCTGAAATTC
CATTTTTCGAAGCTTGCTCCTCAATTCCACATGCTTATCATAGATATCAAGCAGTGCCCTTGCTGTCTGCCCATCT
TTTCTGACCCTGGGACACCACCCTTTATTTACTTTCCCCACAACTCCTTGAAGGTCAAGCCCCCTTGGCTGATACT
CTGGGTTATCACAGTAAATGTGCCCTCTATCTTTTGCAGGTCACTGGAAAGGAGGGTTCCTAAAGCATTCACTCCT
GACCCTGAGGGAGGTGGGTGCACTCACTCCAGGACTCAGGTTCACCACAGATAAGCAAAAAAGTCCTCACATTCAA
AATTGTCCTGGGCCACATGCAGCCCACGAGCCGCGGGTTGGACAAGCCTGCATCTAGAACGTATGCACCATAACCC
CAGACATTTTTCTGTTTTTACTTTAAGGATGCTTTCTAAATGCAAAAGCAGTCATATCCCTAGCAGACAACAAATG
TCAGTTGAATGAATGATCACTGTAGAGCACCTCCCTATTCTAAAGCCCATATCTTTATTAATGTAGCCACAGGGCA
AATGCTGTTTTGTGGCAGCTAAAGAACATAAGTTCACGTGGACATCCATGCCGCCAGTGCTTTTCTCACCAGGGCT
GCTTGTGTGTCCTCCCTCCTCCCACACCAACCCTCCTGCACACTTCAGCACAAAACTATATATTTCTCTTCAGGAA
CAATAACCCTAGCCTTATGGGTACAATTTTCCAACCACATATGAATCTAAATTAGACTCTGCTTTATAAATCTATG
AGTTTGGATTGGAGCCAGCACTAGGATTACTACAACTCAGAGCAGGAAGAAGAGTAGGAGGGCAGAAGAGGAGTTC
CAACAGAAAGTTAACCATGATGAGAAACTATGGGACCCCTCCTCTCTGCAAATGTCACAATCTGGTTCCTTTAAAA
AGATTGGAGAAAAATCGAAAATACCATTAAGGAAAAAGCCCTGGGAGGAAGGCAAGACCTGGACAGGTCTGAAAAT
TTGTCTCTTGATACCACAAGGAGATTTGTATGCAGGGACCACCCTAGGTGACATCCAACTCCCTGTGATCACAGGT
CTTGGTGGGACAAGGTTCTACTGAAGGGCCAAGGACAATGGAGCAGCAAAGATGACCCAGGTGAGCTGTGACCACA
TAAAGCCCATGGTGGCCAGAACAGAAACTGGGCACAGCCCCATCTACTCTCCTCCCCTGCAACAAATCAGCACAAG
AAACACGTGGACTCTGGAAGGTTCTCATGTCTTCCATTTATTTTGTCTCTCAAATTTTAGGAATCTTCTCCTTTAA
TTAACTCATCAACCTCTCATGGCAAGAATTTGAGAAAGTAAATTTATACTCAGGTTCTAATTTTAATAGGGAAGGA
AGAAGTTACAGCTCAGTGCACCATGAAGTTGAGACAGAGATGGAGACACCTCAGCCCCACCTCTCTGGAACAGGAA
AGATGATTGGGGAGGGAGCACAGGTCAGCGTGGGAAGAGGGTCATGGTGGACATGGGGGTGGGGTGGTCTCCCCAC
CTCCTCACATTATGCCTACACGAACACAGACACATGCAGGTGCCTTTGCAGAAACAAAGTCAGGGTTCTTCAAGTC
ACAAAGGGAAGGGCAGGAACAACTCTTGCCTCTCAGTCCCACACAAGGCAGCTGTCTCACACTATAGAAAAAAATA
TTCATGAACAAATTCATATCCATCACAGTGAGGGGTCACACCTTAAACAGCCCATCGCATGCTCAATACATCCAAT
GCAAAGAAACCCCATAGCACAGCTGTGTCCACTGTTCCGCCCAACACCCAACACACATTAGGTCCTCCAAGCTCTC
ACCTTTACAAGCTGTGAGAGACACATCAGAGCCCTGGGCACTGTCACTGCCTGGGGTAGAACAAAAACAGAACCTG
GTCAGATCCCACAGAAGATGTGGCTAGAGGAGGAATTGTGGGGTGGGTGAGCTCCCCCATGGGCTCCCAAACACAA
TATCCCAAGGACCTCAGGCATCAGCCTCCTTCATACTTACTTGCAGCCTGAGTGTAACTCCCTCCTTTTCTATCTG
TGAGAAGAAAATGTCCTGTGAGATACCAGAAAGGAGCCAGGGCCTTAAGGTCCTAGAGGAACCTCCTAGTCTTGGA
CCCCAGAGAAGTTTCCAGAAATGTGTGACTGCAGACCCAGGGCGGGATCAGGAAACATGAAGAAAGCAGGTGTGGG
TCCTGGACCAATAGCCCTCCTGAGGTCTGTCCTCAGGGACCTTCCCCTGTGACTTGTGACTGCTGGGATCAGGTCC
CATCACCGCCGTAATCAAGGTGATAAATCTGTCCTTCATTTTAACAGGTGCTTTACAAAAGAGTAAGTGCTGGCAC
ACAGGGCCCAGGCTGGGTAGGCCCATGATTGTGGATGGTGCTTCCCAGTAATGAGACAGGGCACATTTCTAGCTGG
GGCTTGGAACCCTCAGTGAGACAAGAAATCTCAGACCCCACCCTTCACCCCTTCTCCACCTGAGCTCTTCCTCCTC
CACATCACGGCAGCGACCACAGCTCCAGTGATCACAGCTCCAAGGAGAACCAGGCCAGCAATGATGCCCACGATGG
GGATGGTGGGCTGGGAAGACAGCTCTGGGAAAAGAGGGGAAGGTGAGGGGCCCTGACCCTGCTAAAGGTCTCCAGA
GAGGCTCCTGCTTTCCCTAAGAGACATGACACCCCCATCTCCCTCCTTACCCCATCTCAGGGTGAGGGGCTTGGGC
AGACCCTCATGCTGCACATGGCAGGTGTATCTCTGCTCCTCTCCAGAAGGCACCACCACAGCCGCCCACTTCTGGA
AGGTTCCATCCCCTGCAGGCCTGGTCTCCACGAGCTCCGTGTCCTGGGTCTGGTCCTCCCCATCCCGCTGCCAGGT
CAGTGTGATCTCCGCAGGGTAGAAGCCCAGGGCCCAGCACCTCAGGGTGGCCTCATGGTCAGAGATGGGGTGGTGG
GTCATATGTGTCTTGGGGGGGTCTGACGGGAAGAGTCAGAAAATTCAGGCATTTTGCATCTGTCATGGGACACTCC
ACCAGCACGCATGTGGCCATCTTGAGAATGGACAGGACACCCGGGATGGGGAAGAGAGCACAGAACCCAGACACCA
GCCTGGACACAGGCACCTGGGATAATCTTCTATTCCCTGAGAAGGGAACAGCGACTTCTGGTCCTGACCTGAGTGG
AGGCTGAGGGACTCAGAAGTGCTGGACTCAGACCCCCACACACATTGAGTGTGAAGCAGAGAACAAGGCCTGAGAG
GAAAAGTCACGGGCCCAAGGCTGCTGCCGGTGTCAAAGGGAACCACTCATCAGTATTCGAGGGATCGTCTTCCCGT
CACTCCTTCAGAGATTTTATCCCTTAATTGTGTCAGAGAGCAGGGCGGAACCTCAGAGTCACTCTCTGGTACAGGA
TCTGGAAACCCAGGAGGATTCCTCTCCCTCAGGACCAGAGGGAGGGTGATATTCTAGTGTTGGTCCCAATTGTCTC
CCCTCCTTGTGGGAGGCCAGCCCGGGAGATCTACAGGCGATCAGGAAGGCGCCCCGTGGCCCCTGGTACCCGTGCG
CTGCAGCGTCTCCTTCCCGTTCTCCAGGTATCTGCGGAGCCACTCCACGCACGTGCCCTCCAGGTAGGCTCTCAAC
TGCTCCGCCCAACGGGCCGCCTCCCACTTGCGCTGGGTGATCTGAGCCGCCATGTCCGCCGCGGTCCAAGAGCGCA
GGTCCTCGTTCAGGGCGATGTAATCCTTGCCGTCGTAGGCGTGCTGTTCATACCCGCGGAGGAAGCGCCCGTCCGA
CCCCACGTCGCAGCCATACATTATCTGGATGGTGTGAGAACCTGGCCCCGACCCCGCGGTCAGCCCAGTCCCCCGA
GCCCCGCCCAGCCCCGACCAACCCGGGGGAATTTTTGGCCTAAACTGAAAATGAAACCGGGTAAAGGCGCCTGGGC
CTCTCCCGGGTCAAGGGTCTCGGGGTCCCGCGGCTTCGGGGTGGATCTCGGACCCGGAGACTGTGGGCGACCTGGC
CCGTCCGTGGGGGATGAGGGGTCGTGACCTGCGCCCCCGGCCGGGGTCACTCACCGGCCTCGCTCTGGTTGTAGTA
GCCGCGCAGGGTCCCCAGGTCCACTCGGTCAGTCTGTGACTGGGCCTTCACATTCCGTGTCTCCTGGTCCCAATAC
TCAGGCCTCTCCTGCTCTATCCACGGCGCCCGCGGCTCCATCCTCTGGCTCGCGGCGTCGCTGTCGAACCGCACGA
ACTGCGTGTCGTCCACGTAGCCCACTGCGATGAAGCGGGGCTCTCCACTGCCGGGCCGGGACACGGATGTGGAGAA
ATACCTCATGGAGTGGGAGCCTGGGGGCGAGCAGTGGCTGAGACCTGACCGACCCTCCTCCCGGCGCGGCTCCCCC
GGTCCTGCGCCCCCGCCAGGAGGGCCCCTTGCTTCTCCCCGCAGAGGCGGTTTCCCTCCCGACCCCGCACTCACCC
GCCCAGGTCTGGGTCAGGGCCAGGGCCCCCGAGAGTAGCAGGAGGAGGGTTCGGGGCGCCATGACGGCCATCCTCG
GCGTCTGGGGAGAATCTGAGTCCCGGTGGGTGCGTGCGGGCTTTAGAACAGCGACCGCGACGACACTGATTGGCTT
CTCTGGAAACCCGACACCCAATGGGAGTGAGAACTGGGTCCGCGTCGTGAGTATCCAGGATGAAGGACCCTACGTA
GGTTGGGAGAGGGAGAAAAGAAACTGCGGAGTTGGGGAATCCCCAAGGCTGTGACTCCCCAATCCATACACCGCCT
TCGGGGCCTGAGACCCTGAGAGCCACGCCTGGGGCCCTGGGACTTCGCCCTGACCCCTCTGCTCCTGTGCGAAGCG
CTCTGTCTCAATGTCTCCCTGAGTCTTGGCCCAAGAGCTGTCTGAGAAACAAGGGAGAAACCCTCGGTTCACTTTT
CATCCCATTATCTCTGTCCCTGAACTGGACTCCCTGACTCCCACTCCTTACCTGTCCACCTGGACTCTTCTAGAAG
AAAAATCACCCCATGGAGTTTGGTGCCAGAGAGTAAGCTTGCTCTGGGAATGGAGGTGTAGATTTTTGTTGTTGTT
GTTTTTGTTTTTAATCCGGAAAAGTTGTGCCTGAGTGCATGAGACAGAATAGAGACCAATTTGCTCTTTGTTTATT
AACTACAGTGGGTAGCAGAATCTTGGTAACCCCTGAATGATCAGGAATCTAATAGGTAAAAAAATGTGACTTTGGC
CCCTTAATGTATAAATGTGTCCAAAAGCATTACAACAGGACTCACAAAGCTACTAAGTTGCACTTTCCCAGACAAT
GTATCTGTGACTCCTGCTTGTCTTACTTTAAATTTGCCATCATTCCTTAGCTCTGAGTTTCTGTGTGAGTCCACGA
CATCTCCTCAATACAAAGTAGCCACTGTGTTCCTATATGTTGCAACCAGAAGCCATTACAGGCTTTATTCACCTCA
AAGTTGCAACTGTTCAAGGCAGTCACAATGCCCCTCACTAGTGCTCATGCACTGCCTGTTTTTAGGAAGTATCCAC
TTCTAAGTGTTGTGTATATTTTATAGGAACACTTAGTAATTTTTAAACCTGATTAACATAAAAAATAGTTTTTAGG
CAGACCCACATGAGGTATTAAAAGACAACTGCCAAGGACACCCTGCTAGGCTCTGTAGATTGATGTATTAAAATCC
ATAAAACAATGTCTTTAAACCTAAGAATTCTGCTGCTTTCAAATTCTTTCCTCTGCTCCTTTTCCTCACCTCCTGC
TTCTCCAGCCCTTCCCTCCATCCTTCTCATCCTTCAGGCCCTCCTCTCCCCTTAGTCCCCACCACACTGTCACTCA
TGAATTGTGACACTAGCACTGTCCCATGACCTGCTACGTAACTGTTCTTTTCACAGTGGCCCTGCTCCTGTGAGTC
GGAGTGTGTCATTTCTCCACTTAAAACACTCCAGTGTCTCCACCTCGGTCTTGTGAAGCTTCTGGAGTGTCAGGCA
CTTGAGCATATGAGGGGATACCTCGTTCATTGTAGGGACTAAGTTAATTTTTGTTGACTTAATGAATGAAATATGA
GTGTATTAAATTGCATCACAGATAATTATAAACTGTAAAACACTGAAAAAGTTCAGAAAGATTTTATTTTATGCAA
CTAGTGTGTATATCAATTCATCAATTCATTCGCACACTACCATGCCTGGCAAAACAACACACATTTATCTGCTTAT
AGTTCCTTGGTCAGAAATCTGGGCAAGATGTGGATAGAATCTCCATTCTGGGCTTCCAAAAGCTGTGTTTTCATTT
TGAATCCTCCTTCGGGTTTATACAGAGGTGGCAGAATGCAGTTTCTTGCAGTTGTAAGACTGAGGTCCCTGTTCCT
GGCTAGCTGTCAATGTAGAGAACTGGGAGGGGCTCAATTCCCGGTGCTCACCAGCGTTCTTTCCTACACAGCCCCT
TAATTTTTCTTTTTTTTTTTTTTTTTGAGACGGAGTCTGGCTCTGTAGCCCAGGCTGGGGTACAGTGGCCCAATGA
TCTCTGCTCACTACAAGTTCTGCCTCCTGGGTTCACACCATTCTCCTGTCTCAGTCCCAAGTAGCTGGGACTACAG
GCGCCCACCACTATGCTCTAATTTTTTTTTTCTTAGTAGAGACAGGGTTTCACCTTGTTAGCCAGGATTGTCTCGA
CCTCCTGACCTCATGATCCGCCCGCCTCGGCCTCCCAAAGTGTCGGGATTACAGGCGTGAGCCACCGCGCCCGGCC
CATAGCCCCTTCCTTTTCAAAGCCCATGGTGGAGGAAACCCCTCATGCTGAGTCCCTTTCACACTGTAAGTCTCTA
TGCTCATGAAGAACCCAGTCCTTTCAAGGGCTCACCTGATTAGGACAGTCCAAGCAGGATAAACCCAGCATAAAGT
CAACTAATTGAGACCCTTAATTATATCTGCTAAATCCCTTCACAGCAGCACCTACATTAGAGTTGGTTGAATAACT
GGAGGAAGGTGAATGACCAGGAGCTGCTTGTTGAGGCCATCATAGAATCAGCCTAGCAAGGGTTGGATCTTCCTTT
TGTGTTTAATTGGGACACAGTTGGAAAGTGAAGTTCAAGTAACGTGATCATTGTGAATGGTCATAAAATACATCCT
CTTCAGCCATGGAAATTGTCCTTACTTTTTAAAACTAAGTTACATGTTTAATATCTTATAATTAATTTAGGCCAGG
TGTGGTGGCTCACATCTGTCATCCTAGCACTGTGGAAGGCAGAGGAATGGAGATTTGTTGACTCCAGAAGTTCAAG
ATCAGCCTGGGCAAAACCTCCATCTATACAAAAAATTAGAAAATTAGCCAGGCATGGTAGTTCATGATGGTAGTCG
CAGCTACTCAGGAGGCTGAGGTCAGAGAGTCCCTTGACCCCAGGAGTTCGACACTGCAGTGCATGGTGATCATGCC
ACTGCATTCCAGCCTGGGTGACAGAGCAAGACCCTGTCTCAAAAATAATAATGATGATGATGACACATTTAGAGCA
AATGCAATTTGATGTGTAATAATACATCCTCTCTTGTGAAAATATATTATTTACTGTTGCATAACAAATTACGTAA
AACTTAGCTCGAAACAACAAATGTTTCTCATCTCCCACAGTTTCCAATCGTCAGGAATCCAGGAGAGGTTTCCCTG
AGTGCTTCCTGCTCAGGGCCTCTCACAAGGTTGCACTCCAGTTGTCAGCCAGGGGCCGCAGCATCTGAGGGCTTCG
CTGGGGCTGGGAATTCACAAGAAACATGGATCAGTCACATGGCTGTTGGAAAAGGCCTGGTTCCTTGTTGTCTGTT
CCCAGAAGGCCTCAGTTCTCAGTCATGTGGACCTTCCTGCAGGGCTGCTTATGGCACAGCGGCTGGCTTCCCCAAG
AGCTCATGATCCCAGATTCAGAGAGAGAGAAGGTGGAAGCCGCAGTTAGTTTTATGTTCTACACCCAGAGTCACAA
ACTGTTATGTTGGCACTATTCTATAAGTTAGAAGTTGTATTAGTCCATTCTCACACTGCTATAAAGAAATACCTGA
GACTGGGTAATTTATAAAGGAAAGAGGTTTAATTGACCCACAGTTCTGCATGACTGAGGAGGCTGCCCCAGGAAAC
TTACAGTCATGGCAGAAGTGGAAGCAAACCCGTCCTTCTTCATGTGGTGACAGGAGAGAGAAATGCACAATGAAGC
AGGGAAAAGCCCCTTATAAAACCATCAGATCTCATGAGTATTCCCTCAGCATCAGGAGAACAGCATGGGGGCACCG
TCCCCATGATCCAATCAACTCCCATGAGGTCCCTCCCCCAATAGGTAGGGATTACAATTTGCATAACAACTCAAGA
TGACATTTGGGTGGGAACACAGAACCAGGCCATTTCAGAAGTGCCTCATTAAGTCCAAGCCACACTCAAGAGAGGG
AATTAAGCTGCACCTCCAGAAAGGAACAGTATCAAAGGATTTGAATATATGTTAAAAGCAAAATTATAACTATTGT
TTCAGGTTTCTGAAAATCAAAGTCTTCTTGTATCTAATTATTTTTCATTAACTCTTTAAGCTTGTCTTTTAATTTA
ATTTTTTTTTTAAGTTCCAGGGTACATGTGTAGGATGTGCAGGTTTGTTACATAGGTAAATGTGTGCCATGTTGGT
TTGCTGCACCTGTCAACTCATCACCTAGGTATTAACCCTGGCATGCATTAGCTATTTTTGCTAATGATCCTCTCAT
CACCACCCTCCCCCAACAGGCCCCAGTATGTGTTGTTCCTCTCCCTGTGTCCATCTGTTCTCATTGCTCAGTTCCC
AGTTATATATGAGAACATGTGGTGTTTGGTTTTCTGTTCCTGTGTGAGTTTGCTGAGGATAATGGCTTCCACCTTC
ATCCATATCCCTGCAAAGGACTTGATCTCCTTCCTTTTTATGGCTGCATAATATTCCATGGTGTATATGTACCATA
TTTTCTTTATCCAGACCATCATTGATGGGCATTTGGGTTGATTCCATGTCTTTGCTATTGTGAATAGTGCTGCCAT
GAATGTAAACATGCATATGTCTTTATAATAGAATGATTTATATTCCTTTGGGTATATACCCTGCAATGGGATTGCT
GGGTCAAATGGTATTTCTGGTTCTAAATCTTTGAGGAATCGCCACACTGTCACTGTCTTCCACAATGGATTAACCA
TTTACTTTTCCACCAACAGTGTAAAAGCATTCCTATTTCTCCGCAACCTTGCCAGCATCTGTTGTTTCTTGACTAT
TTAATAATTGCCATTCTGACTGGCATAAAATGGTATCTCATTTGTCGTTTTGATTTGCATTTCTCTAATGATCAGT
GATGGGAGCTTTCTTTTTCATATTTTTGTTGGCCACATGTATGTCTTCTTTAGAAGTGTCTGTTCATGTTCTTTGT
CCACTTTTTAATGAAGTTTTTTTTCATGTAAATTTGCTTAAGTTCTTTGTGGATTCTGAATACTAGACTTTTGTCA
GATGAACAGATGGCAAAATTTTTCTCCCATTCTGTAGCATGTCTGTTCACTCTGATTGATGATAGTTTCTTTTGCT
GTGCAGAAAAAAATTGCCTAATCTGATTGCTTTTCATTTACAAAAATGAGAATCTCATACTTCAGTTAACACATCT
AGCTTAGCATGATGCCAATAATTTTGGCTTGATGCTGTAGCCCGAAATGGTTAGCTCGAATTGAGAGACAAGTTAT
TTTCAAAAACTGGCTATCCGATGGGCATTCTATAACACACTTAACATCTTTTGTTGTGGTATTGAATGAGATGTGA
TAGAAGTGATTTGGACACCACTATGATTCTAAATAGCACTGCTATTATGTGCTTCTGAATTTTTTTTCTTTTTAGC
AGTCCTATTCTGTTTCCATTCATATTTGCTATTCCATCACTGGGTATTCCTTCCTACTCTTTTCATGTTTCATTTA
TTTTGATCAGTGATGTGCATTTCCAATTCTGTAAAAGTTTAATTCAGTTGTATGTGTGGTAAAATGTAACATAGAA
TCTTTGCAAGATGAAATTACCTTGCACAGCATATTGAACATTGTATTATTGAAAATCTAGAGAGATGCCAGTGAGC
CAAGGATCGAATGACCTATATGTAGCCAAGGCCGTTTTCATGGCATCCTGGTCCTCTCTGTCACATAGCTCCTTAC
AATTCTCTGTGGTTTTCTTCCAATGTAAGTAGTTCTTGTTAAGAATCTTTGCAATGAACTTTGAAATTCTTTTCTT
TTTCATTGCTATAAAAAACAGGTATATTTTAATTTGGAAAACTCTGTTTAAGGAATTATAATCAAGTAACTACCAG
GGGGCCCAGCACTGGTGTAAAACACAAAACTGTAAAACTACCCTGGGCTGCTACCCTTCGAAATGGGGTCTTTTCA
AAGTTAGTAACAATGAAGCTGTCATTCACACGATGCATGGCTGAACAAAAAGACAGAGACAGCGGGTGCTGCATTT
TATTTTTGTAATGTTGCCTTGTCCACAAGACACTATTTACACTTAAATCAATTAAAATTATATTAATTTTAAAAGT
CAGTTTTCCCAGTTTCTCATTTGTACTAGCCACATTTCAAATACTCAGAATTCATGGGTGGCCGGTGACTGACGTA
ATGAACAGCACAGATATAGGCATTCCATCACTACAGAAGGGAGCTGGACAGCAGTGTGCCAATGTTTTAAGGACAG
AATCCTGAGTAGAATTATAGGATGATGACAATTCCGAGTTCCCATTCCAATTCATGGTCTCTTCTCTGATGAGTGG
TGTAGGGGGAAATCCATCTCTTTTGTGAGTAAAGGTTAATTTTTCCAACTACAGATAAATAATTAACATTACTTTT
TTTAAAACCAGTTTTACACTGTTAAGTTACAACCAAAAGTGAAGGGCTTGAATTACATTTACATTTTAACACATGG
TTTTAAAGGGAAGATCCGTGTGAAAAAGAAACAAAACTCAGAAAAAAAACCCAGAAGTTTTCATACCAATTCCCAT
AGCAATAACAATCCCTTTTCTGTCGTTAGAGTTCACCCTCATTCTGATTTCATAGTAATCCTGTTCATGTCTTTCT
TTAAAGCTTTACCCCATGCATATATTTCTAAAACAACACAGGTTATCTCAAACTTTATATAAATTTTGAATTTTTC
ATACAAATGGAATCATGCTGTGTATATTCTTACAAGACCTCATTTTAGATCAAATGTATGTTTGCTAAGTTTTTAT
AGGTGTCTGTATTCATGTTTATTATTTGTAACATTTCATTGAATAAATACATCATATATTAAATTAACCCTTCTCT
CGTTTATGGTCATTTCATTTGTTGTCAGATTTTTTAAAATTACAAGCAATGCTGTTACAAGTCTTCTAGCACTAGT
CTGTTGGCACAATGGGCACATATCTGTCAAAATTATACACCTAGGAGTGGAGTTACTGGTATATGTGTATGATATC
CCACTCAAAGAGATGATGCCACACTGCTAGATAAAGTGGCTGTGGCAAGCAATGTGTGGTAGATCTTGATGGTCTG
TATTCTCATCAGTGTTTGGAAAACTCAATAATTTTAATATAGAAATTCTAGTGCATTTTTCAAAATATCTCATTAT
GTTTTCCTCATTAAACTTTATTGAGATATAAATCACATACTACTCACCCATCTAAAGTATGCAATTCAATGGTTTT
TCACATATTCAGAGTTTTAAATCTGCATATTTAATTTTAGAACATTTTCATCATCCCAAGATAATCAACGGCTTTA
TAATATGTCTCTCTATACAATGAAGTAAATCCATAAATTTTGAGTTTTCACTTCAGGAGTGTTTTTTCTATCATTG
GCAACTTTCCTAGCTTGACAGGCACCTTTCCAAGTACCTTTAACAATTTTATTTTTAATAGAGATTTCATTCAGTA
ATCAGATTTGGAGAGCACTGACAAATTTTGCCACATTGAGACTTATAATTCATGAACATGTAATGTTTACTTATTA
AATACTCTTAAATAAAGTATTGCTGGTTTTACTTGTGCATCTTTTAGTAGATTTATTCCAAAGATCTTGAAATATG
GTGCTAGTGTTCACAGTATCTCTTTCTTTGTTGAATTTCAGTGTTGGTTGTGGTATGTATAAATATAAATAATTTT
ATATAGTTTTAATTCAATGACTTTGCTTAAAGCTTTTATTAATTCATGTAGTTGATATATTCTGTTGCCTGATTGC
ACTAGCTAGGAAATCCAGTGTGATTTTGAGTGCAATGGAGAGGGCAGGTCTGCTGGTATCTTCTCTGTGCACTCCT
GATCCACACTCCATGTACTCTCCCTGCTCCGTGTCCTGGACACTGTGCTGCATGGCCTGCTGGACCACAAAAGGCA
GCCTTGCTTTCTGGCTTTTATTTGGGATCGGCTAAATGGGAGCATCATCACGGAGAGAGTGATGAGGAGCACTGGT
TGGAGGGATTTCTCCCCATAGCTTTCAGTATTGACATAGGGAGAAATGAGATGTGATTGGCCTGGATTCATCAATA
TGGGATCTTCACCCAGTATCTTAGATTAAATGTGTTATTTTGAACACCTAAGAGTGATGTTAATAGTCAGCCAGGT
TAGCTAATTAAAACCCGAATTCAACTGTGGCCTAGCACAGTGTTTCTCAAATTTGACTGAGCTTTCCATCACGTGG
GAGATTTTAAATAGCCCCAGGTCTAGATCAACATGTAAAACCAATGAAATCTAATTCATCAAAAGTAGGATCCTGG
AAACAGTATTGTTCCTAAAACTCTCCAGGTTTGACTCCAATCAACAGCCAAGTTAAAAACTAGTAGCTCAAAGCAG
TGGTTCTCAATATTTGGTATGTATCAAAATCATCTGGGAATTGTAGCAAAACTACACAGGCCCAGATATTATTCTA
ATTTAACAGGCTAGAGCCTTGAGTTTATTAAAACAAATCATCTCTCCAGATTTTGCTATATGCAACAGTTTGTGAA
AACCACTAGCCTATATCAACAGAATCCCTATCACAGGCATACTGCAGAGGAAAGTGTCTGAACACAGCTCAGAGAG
ACGGAAAAGTGGGAATGTATCCATTATGTGCAACCTACTTAACATCTCCTAACCACATCTCCCGGGAGGGCTGAGA
GGATGCCCCCCTTACCAAGGCATTAAGGTGCCACATGACACATACAGGTATAAAAACTGCTAGGTCTGCTGAGCAG
TCACCAATCTTGAGTCCTGGATATTCAGAAATTCTTATCCAGTAGCTACACCAAAGCCAGCTGATATGTCAGAGCC
CCTCAAATAAGCAGAAGGCTGGTGCCCTTGAGAAAGGACCCTATAATTCACTCTGAGATAAATTCTTTTTACAGGA
CTTTCCCAGAGGAACATTTAGTCATTTATCGCTGTCCACTGGGGAATGGAAAACCCCAAATAATCTGGGAGCTCAA
CTTTAATGTCTTTAATCCTGTCAAAGATTACCCTGGGGTTAATACTTTGTTGTAACCTGCATAAAAATCTTTCCCT
ACACTAAAGTAATGAAGATATTCTACTATATTACTGTAAAGAGATGAGTACAATCACATTTTTTTACAAGAGATGA
GTACAATTGCATTTTTCCAGATAGATAACCAATTGTTGTTAAATTATTTTCTAAATCGTTTTCCTTTACCCACTGA
TCTGTAATGTTGCCTCTGCTCTACATACGTTCAGGGCTTTCTATTCTGTTCCCAAGTTCTAGTTTACTACCCTGTC
TCAACATCTCGACTCTCTTAATTAAAATAACTCTAACATAAGTCTTAATATCTGCTAAGGAAATTCTCTCTGCTTA
TTATTCTTATTTAGAGTGTCATAGCTACTTTTTCTCTTTCATATTTCAATATAAGGCTTAAAATTGGCTTACTATG
TGTTGCAAAAATGCTTGATAAACATTTGATTGAGATGTGTTTGAGTTCTTAGAGTACTGTAGGAAATTAATGTCAT
TATTATATTGAACCATCCAAGCCAGGATCCTGGTAAATTGATTCAGTTAATTCACTGTCTTCTTTAATTACTGTCT
TCTATTATTACTCAGTAGGGTTTTTTAAATATCCCAATTGATATCTTGTACAGAATTTGTTAGGTTTAGTTTTAGA
AGTTTATATGTACGCACTTTTGCTGAATCAATTTTTTAAAATTTTCATTTTATATCTGTGCAAAACAGCTCTATAA
TTTCTTCTACCTACCAAGCAAAAATATCTTATTAACTTCCGGATCACGAGGTCAGGAGATCAAGACCATCCTGGCT
AACATGGTGAAACCCCATCTCTACTAAAAATACAAAAAATTAGCCGGGCATGGTGGCGGGCGCCTGTAATCCCAGC
TACTCGGGAGGCTGAGGCAGGAGAATGGCCTAAGTAAATCTGGGAGGCGGAGCTTGCAGTGAGCCGAGATCTCGCC
ACTGCACTCCAGCCTGGGCGACAGAGCGAGACTCCNTCTCAAAAAAAAAAAAAAAAAAAAAAAATATATATATATA
TATATATAAATATATTTATATATATATGTATCTTATTAACTTCAAGAGTGTTCTAATCATTTGTATTTTCTGCATT
CAGCATATTACCATTTGCAAATTATAAGAGTTTTGTGCCTTCTTTTCAAACCTTTTTAAGTATATTTCTTTTCTTC
TTTATGACACTGGGAAGATCCTCAATTACAATTTTTAATAAGAGCAGTGAAAACTGCTGGACCACTCAAACAAAAA
TTGAGCTGCCACTCCTGAGAACTGACTGTGTTGCTCTTATGGATCAGCCCTTTGGGACCCATCCTAATCCCAGGGA
TTTACAGTGCCACATATGCACCCGCAGCCTTTAGATGTAGACTTTATCCACCTAGCCCTCAAAGGTCTCAGACACT
GCCCAGAGCAGCCAACATCACCCAGGACAAAAGCAACCCTGCAGCTCAACTTGCTCTTCTAGATTTCTGCCTTCTC
CTGGTTCCTGGCCTGGCAAATCTTTACTGCTTGGTTAGCCCCTTCATACTTTCCATCAGATTTTTCAAATATGTAC
CCTTTTCCCACTTGTCCAGTAGTACTCAATGGGAGGGATGATCCAAATTACATAATTAATAATTGCTGAAAACTAA
AGTACAATGTACAATTTTTATGTCAATGTCACATGCCAAGGGAGAAAATGCATAGGTATCAGTAGTGACTACTTAA
GCATGTGTAACTTAGAGGTAATAAAAACAAAAATGTTAGGTAATATCTATCTATTCCAGATTAAAATCTACATCAA
TAAATTCAAACTTCTAGGAAAAATATCTGCATATGTAATATATGACTGTGACTCCGATAGACGAACGTGGGAATAT
AGTATGAAGATACTTCTCATCGTCTTCACTCTGTGAGGCAGTCAGTGTGAAATGAGAACCAGGGATTCAAAATGTG
TTGCAAGACTATGAAAAGCACTAGAGTAAGTTCTACACAAATAAAGCAATCTCCTCATAAATTACACTGGGCTTAT
CTGTGAGATGCATGTCCTTATCAAAATGGAGTTAATTCTAGGTAGGTGATCTTGTTTAAATAAAATGGACTCTGAC
TCACTAGCTACTGGTCCACCATCGGTTTTTCCCTTTACTAAAAGGGAGGCCCCACACTGAGCCAGCATTTCCCAAG
AGGAGGTGTCTTGCTGACATCAACTTCCAACAGGGTAAATTTTCATGGGAACCCAAAGAGAAGAGGAAGACCTCAA
GAAAAAGGGCCTAAAGAAATGACAAGAATGATAACATTTTACACTTTTATTGTGCTATGTGTCAAATAGTGGTCTT
CGCACTTTAAAAATGCCAATTCATTTTATTCTGACTACACATCCATGGCTTGTGCCTGTTATTTTTCCCTTTCATA
GTTAATGCCATAGGAATGAAGATCACACAAAAGTAGGGATGGAGCCGGGTTTGCACCCAGACAATTCTGCCCCAGG
GCCATGCTCACACCACTGCACTTTTCCAGAATGTGAGGGTGGGCGGAGAGTCCAGCTCAGGGAGAGAGATTGGAGT
GAGAGAAGAAGGAGAGGGGGCCGACTGGATCACTGTGATGGTCTGGACCCACGATTCCAGGAGAGAAAGACAAGGA
TTATGTCACTCAGGGAAAATATCCAAAGTCTCTGGTTTAAACTTAAGCATCTCCAGCTCCAGGACAGGTGGCTGGT
GGGAAGACAAACTAAGCCAAAGCCACAGCTCTGAAGATTTCCCTCTCCTGAGAATTCTGCAGGGGTTTCCCTGACC
TCATGGCCACCTCTCACCCTTGGCTCTTGTTTTTCCCCCAGGACTGATAAGGGCGTCCAGATGGGACGCCTTTATC
AGTTGGTCTCGGGTTCTAGAGAAGCCATCAGAGTCAGTGGAGGATGAGCTTCATAAAGTGGGAGATCTCCAGGATC
CTTCCTGGACTCCAAGATACCCAGAGAAGCCGGATCCTGTGTCCCGGAACCCCCTTCCTGTTCCTTTGTGGGCCCT
GACCTTAGGGAGAACTGGGCTGGTGAGAAGATCAGGATGAACTGGGCTGGGGAGGCAAGAGGGAAAGGGCGGCTTG
GAGGGCTTAGTAGCTCCTCTCGCGGCGCCTCCGTCCCGGGCTGGGATCTGCAGACTCCTCAGGTCACTTCTCCCAG
AGCCGCCGCCCCAAGCCACCCTCCCCTGGTTCCCGTCCCTCTGTCCCCTCCCCAGCTCCCCCCACACAGTAAGAAG
CTCCCAAGTGAGCGGCTCCGGTGCCGGGCGGGACGTGGGAGGGAGGCTGTGTCCCCGCGGGGAGGCAGGGCGACGG
CGTCCAGGAGAGAGGGGAGCGGGGCGGTGTCCCAGGCACCCTGCGCCTCTCCCGCCCGCAATGTGCGGGTTCCCGG
AGCCCAGGCCCGCACCCCGGGTACTTGAAGGCCGCCGGGCTCCGCCTTCACCACGTGGGCCCTGAGTTCCTCCGCG
GCGCCTTCGTGGGCGACACGCGATTCCTGAGCTTCCACAGCCGCGGCGAGAATCAGAGCGTGAAGCGCGGGTGGAG
AAGATGGCACAGGGCTGTGGGCGCCCTGAGGAAAGTTCTCGAAGACCGCAGGGAATGTGTTACTGAGGGGCCTGAG
GTTCCTGGGCCCGCACCACCACCAGGGTCAGCCGGTGAGTGACCTCAGACTCCGGGCACAGGTGTGCCCCGCCCCG
ACCCCAGAGTGACCAGCCCGGTTCTCCCAGGGATGGGGGGGCGGGGCCTCAGCTGATCCCTCTCAGGTGGGGAAGC
GCTGGGGCCATTTTGCTCCAGGTTTCTCCTTCAATCTGGATCATGACTGACTTGTTCTGTGACCAATGACAGAGGG
AGGAAGGAGCTGGGGTGGGCGGGGCGACCTCCCTCCCCTCCCTGGCCCCGCCTCCGGCTCACCCTGGGGCAGGTCC
AGGGCACCGCACCTGCAGATGCTGCTGTCTGGCTGGGTGGTGGCTCCGGACAGGAGCTTCCTCGAAGGACACTTTA
AGTCCGCCTACGACAAGGAGATTTCATGTCCCTGAACGAGGACCTGCAGACCTGGACAGCGGCTCAAAGAGCGGCT
TGGAGGATCCATCACACCTGGGAGAGGTTCTGGACCGCGGAGGCCACAAGGGAAGCCCCGATGGGTCACTGCACTA
GGTGGCTGCTCAGACACCTGGAGGATGGCAAGGAGATGATGCTGCCGGCAGGTACTGGGGTCCTCCAGGCCCCACG
TCAGGACCTCTTGGAGCTGGAATTATGTTCCTTGGAGGCATCCATATTTGAGGAAAGAGGAGACCGAACAGTCTCC
GTCTCCTCCTCCTCCTCCTTCTCCTCCTCCGTCGCCGCCTCCTCCTCCTCCTCCCTACTGCGGGGTTGGGGATTCT
GGCCCAGGCTCCTGAGGCTCTCCCATGGGTGGGTGGGGAGGGAGGACAGAGAAGAAGGAGTGCAGGGAGGTAAATA
GGAAAGGACAGTAGTTACCCTTGTCAACAGTGATGTACTTGGGTCTAGCTTCACCGGGAATCTTTTTCTTTCCTGG
CCCTCTGTGCCCAACAATTACATATAATCTTTAACCCAACCAGAAGATAAAACACCAGGAAACGGAATCGGCAGAG
ACACAGTAGATGAAAGTACCACGAAGGACTGCTAGGTTCTAATAGAAAGTAGCAGATGTGAGTATCAAAAGTTATA
AACCTTTTTGCAAATCAGAAAAGAAACAGACTTTTTTTTTTTTTTTTTTTTTTTTTTTTTTTTTTTGAGACGGAGT
CTCTCTCTGTCACCCAGGCTGGAGTGCAGTGGCGCGATCTCAGCTCACTGCAAGCTCCGCCTCCCAGGTTCACGCC
ATTCTCCTGCCTCAGCCTCCCGAGTAGCTGGGACTACAGGCGCCCGCCACTAAGCCCGGCTAATTTTTTGTATTTT
TAGTAGAGACGGGGTTTCACTGTGTTAGCCAGGATGGTCTCGATCTCCTGACCTCATGATCCGCCCGCCTCGGCCT
CCCAGAGTGCTGGGATTACAGGCGTGAGCCACCGCGCCTGGCCGAAAAAGACATTTAAAGACTGAAAAGTGGTATC
ACCAATGTTAGAACCAAAAAACTGGACCTGAGGGTGCATCACCCGAGGGGGTTAAGTTAAACTCTGTCCCTGTTCC
TTCTAGAGCCCCGTGGAGATTCCTGCTGTGCAGCAAAGCCTGGGAGGGGCCCCTGGCTGTGGAAGGCAGGCTGAGA
CGTCATAGGTGAGATAGGAGGAAGGGGACCTGCTGGACTCCCGGGGACCTGCTGGACTCCCGGAGGCCTGGTACCC
AGGGAAGGCCAGTTCCTAGGGCTGCCTCTGGACTTCAGAACCTTGATGTTAAGTCATATTTTCCCGCTCCTCCATG
AGCTTCCAGGAGCAGGGCCACATGGGGCTCAGAAAGTCCCACAGTGCCTAGGAAGTGGGTCTGTCTCTTGTGGGAC
AAAAATTCTCAGGTCTTTCTCTTTCAGATTCTTAAATTTCTTAAAAGAGATTGAACTGGAATTACCCAGGCCCCAA
ACTGAATCAAGGAATTGGGACTGTGTAATGTGCGTGTGTCTGTCTGTGCACATGTGGATGTATAAACAGGTATGGG
CATCTATGTGTATGTGCGTGTTTATAAGTGCGTATATGTATGTATCTGTACATATTTATGTATATAAGCAGGTATG
GGTATGTATGTGTGTATGTGTGTGTGTTACTCTGTGACTTCCTCATACTTGGCACCACCCTGGCCTTTTCCTGGTT
GCCCCCCACACCATAGGTTGTAAGGGACAGAACAGCGCTCCACCTGCAGACATAGAACGGGGGTCAGGATAAGTGT
TGGGGAGAGCTGGTTTCATCACACCTGGAATAAGGAAACAGGACCTAAATGGATGGGTCCCACTCTTTTACAGAAT
TGTACCTTAAAAAAAGTGTCAGTCACGTTCCTAAGGGATTTACATGAATTAAATCATTTAAACCTCACAAGAGCCC
TATGACCTAGAGACAGCTATCAGCCCCATTTTACAGACAAGGAAACTGAGGTCACTAGGAAGTTTAGTAACTGTCA
GTGGGCTGACTCATGACTCTAAGGAGACAGTCTGACTGCTGAGCCCAAGTAACTGCTCTGTCATACTCACCCTCAC
ACCCACCTACTACTGGCTCCCTGTGGGTCACCTCCTCCTGTGGGCTCTGACCAGCTTCCCTTTCTTCTCCAGGCAC
AGAGACTGGACTGAAGTCTATTGCATTTGCCACAGTCCCGACTGGGGAGTCATGGGGTTGAAGTGACCCATAGGCT
GATGGAGTGTGCTTGCTGGATGCGGGGGAAAGGGAGATTTATATCTGCCCTGGAGGGAGTCAATGGTGTTTGCATC
ATGGCATATGAGGTGCACTATCTAGAATGCTGAAGGGTAAAGACCAGCAGGGGCCATGCAGAGTCTATTCATCACG
GTTTTATTTCACAGCCTGCATGGGCCTGAGAGTGGTGGGGTGTTCACTCATCTCCATCCAATCCCTACTGGTTCCT
GACCTCTCTTCTTCCAACTGGTAATACCTGCACTCAAGGTTGAACCATGAGGAGGAGTCTACCCACACCCCACACT
TCCTCCTCTCTGCCTGCCACTCCCTAGTGGGGCCTCCTGGATGGTGTGAGAGGAGAGTGACGGAGATAGTAAGAGA
ACTTGACATTCACACTGGTAAAGAGAAAGTGGGATGTCAACCTATGAGAAAGATAAGGGCTGCCACAGGGAAGGGG
ATGAGATAAGTTCTAACTGGCCCCAAGGGTCGCTTAACTCAGTGAAAGCTGTGGGAGGTGTTTTCAGCTATCCATT
GCTATGTAAGAAATTGCCACAAACTTAGCAGCTTACAACAACACACATGTATTATCCCACATTTCTATGGGTCAGG
AATCCAGACACAGCTATGCAGCTTTCTCACATGATTTTCATCAGAGTGCCATGGCTGGGTCTTATCTGGAGGCTGA
ACTGGGGAAGGATCCACTTCCAAGCTCAAGCAATTGTTGGCAGGATTCATTCCTCCATGCCTGTTAGACTGACAGC
CTCAGTTCCTTACTGGCTGGTGGCCAGAGGCTGACTTCAGTTCCTTGCCACATGGGCCTTTCTGCTTCATCAAAAC
TTGCAAACAAAGAAGGTAGAAGACAGAGTCATCTAGTAATACAGAAGTGCGGCTGTAATATGACCTCATTACGGAA
GTGACATCATTTTTGTCACTTTCTATTGGTTAGAAGCAAGTTTAGGCCCCATCGGTTCTCAGCTGGATGAGATGCC
AAGAGACAGGATCACCGGGCATCATCTTAGGGTCTCTATACACAGGCTGGACCATGACTGAACAAGATCATTACAA
ACCCGGGTCCTGGACTGGGCCAGATGATCTGCAGAGCTGAGCTGTGTTTATGAAATCGAATCCCTGGAAACAAAAA
TCAAGAGACGCCCACTGGAAAGGCTGGTCTTGCATTCTGTGTGAAAGCTAAGATGGCGTTCTCTCCAAATGTTTTT
CTCTATTACTTTTCAAACCTCCTGCATCTGTAAAGAAGCAACACCACTAAGCCTATCTGGTTTTTTTTTCATTCGA
GTGTCATACTGTTAGGCAAATCTTCTGCTTTCACTATTTCTTCAAGCTTGTTTTCATGCAGAATCCCACGAACTTC
TGGTAATTACACTTTATTGATTTCTTTTCCTTCACTCAGTTTATTTTTTAGTGATTTTATTTCTCTTGTATGGATA
AATTAGTCCATTTAATTAGGGAATTTGTGTCTTCTAAAGAAATATTTCTACTATTTTTTGGACCATATGGGCTAAG
ATCCTCTTGCCAAGCCTCAGCTCACTTCCCACATGGACACGACAAAATTACTGGGGGTCCTGGAAAGTCGGGGATT
GGCATAGTTCTTAAGCAGGATCAGACATGTAAGTCACAAAGATATGAGGCAGATACCTTTGATTTCCTTATGACCC
TGATGACAGCATACCTGGCACTCAATACCTGTCTGTAGAGGTGAACTCAACTAAACTCCCTGATCACTCAATACCA
TGTGAGCCCAAGGACCTCTTATGTCATGGGCCAAATTCCCTTGTTTTATGCCATTTTTCCAGATGGGGTGCTGTTC
ACATGGGGACGTTTCTCCTTTTCCACCTCATTTTCCATGTGTGAAACACTGGGTCTTGATTACTCAGATCTCTCAG
GGTCCTTTCTACCTTTTCCTCCATCCTAACATTCTCCCTACAGTTCCCGCCAGGGTTTCCCCGAACCTCTTCTTCT
CCATGTATTCAGTCTAGCATCTCTCAGTTCATTCTTCACACTTCACCAAAGTGATATTCTGGAATGCAGATCTGAT
CAAGGTTCTTCCTGACTTCAGTGCTGCCAGTGCTGCACATTGTCCTCGGATAAAAGCCAAAGGTTCTCCCCAACCC
CACAAGCCTTAGTCATTGAATCTGCTCACTGCTCCAAGCTTGCTTTTTGCCCCTGTGCTCCTCTTTTTCTAATCTC
TACCCTGAACTGCTGACAGTTCCCCTAGACTCCCTGTGTGTTCTTTCTTCTGAGCCTTTGTTTGTGATACTGATTC
CCTAAAAGATTTCTCCTGCCATTCACCTGGATTAACTGTGTTCCTCATCCAGTTCTCTGTTTAGACATTGCATCCT
CTTGGAAGCAGTTTTCCCGCACTAATTATAGTAGCCTGCATTTAGCTGTTTTTTTAGTCCCCTTGTGGAATCTTTG
ATGTTTCACTCATTGAGGGCTAGGAGACCCTGACACATAGTAACTGCCTAATAAATGTTTGCTAAATAAATGTGGG
GTTCACCATGAGATACCACGTTATACATGCAAGAATGGCATTAATTTGGCTGGGCGCGGTGGCTCAAGTCTGTAAT
CCCAGCACTTTGGGAGGCCAAAGCAGGCAGATCACGAGATCAGGAGATCGAAACCATCCTGGCTAACACGGTGAAA
CCCTGTCTCTACCAGAAATACAAAAAATTATCTGGGCGTGGTGGCATGCACCTGTAATCCCAGCTACTCGGGACGC
TGAGGCAGGAGAATTGCTGGAACCTGGGAGGTGGAGGTTGCAGTGAACCAAGATCATACCACTGCACTCCAGCCTG
GGAGACAGAGTGAGATTCTGTCTCAAAAAAAAAAATGGCATTAATTAAAAAGTCAAAAAACAATAGATGTTGGCAT
GGATGGATTACACTGCTGGTTGTGAGAATGTAAATTAGTACAACTACTATGGAAAACAGTATGGAGATTCCTTAAA
GAACTAAAACTAGATCTATCATTTGATCCAGCAATCCCACTACTGGGTATCTACCCAAAGGAAAATAAGTCATTAT
ATGAAAAAGACACATGCACACGCATGTTTATAGCAACACAATTCATAATTGCAGGGATGTGGAACCAGCCTAAGTG
CTCATTGACCAACCAGTGGCTAAAGAAAGTGTGGTGTATGTACACCATGGATTACTACTCAGCCATAAAAAGGAAT
GAAATAATGCCTTATGTAGCAACTTAGATGGAGCTGGAGGCCATTATCCTAAGTGGAGTAACTCAGGAATGGAAAA
ACCAAATACTGAATGTTCTCACTTATAAATGAGAGCTAAGCTATGAGGACACAAAAACATGCAAAATGACATAATG
GACTCTAGGGGCTTCTTGGTGGAGGTTGGGAATAGGGTGAGGGATAAAAGTCAACACTGGGTAGAGTACACTGCTC
GGGTGATGGTGCACCAAAATCTCAGAATTCACTACTAAAGAACTCACACACATAACCAAAAACTACCTGTCCCCCA
AAAACTATTGAAATAAAAAAATTTTTTTAAATAAATGTGGGGTTGATTTGTAATTTAAAATTTATTTTGAATAGCT
AATACAAGAACAATATTTAAAAGATTATAATAATAAAAATGTGTATAGTGTGCAACGCAAATTTCCTTCTCCACCC
CATCCACAATTCTTCCAAATTCCTTGCCTGGATGCAACCACTGTTACCAGTTTCAGCAAGAAATTAAGACAGAAAT
TAAAGTTCTGCTCCTCATAATAATCACGATCAATTATCCCTGCTTATACAGTAACCCCTCCTTTTCCCAACAGCCT
CCTAGCATGAGGAACCCAAATTGAACAGGTAACAAACATACCCCTAAACGTAATGTAACTTGTACTCTGTTCCATG
GTAGGAACTTCCTTCTCTGGGATCCCAAACTCCTAGACCAGCTGAGTACGTTGAGGGGATGGGAAGCACCAACACC
CCACGCAGGTTACCGGAAGTGCTGGTGAGTATAGTCATTCCTTCTTCCAGCACTGGTGGTTAGAGACTTACTCTAC
CTGTTGGAGACTTGGCACCATATAACAGCTGCTGGCGTAACCTTAGTGTATGCTTCATCCTGAAGGACATTACCCA
TCCTGATAGGATGTGATCTTCAAGATAAAACCCAGCTGCACCCTCAAGAGACCATTCCACTGCTCTATCAGCTCGC
AGAGTGTGTGATGCAGTATGGGGTAGAACCAGGAGATCTCATGGTCATGTGCCCACTACCAGACATCCTTCGCTAT
AAAGTAGGTCCCTTAGTCTGGGGCAATGATATGTGGGTCCCCTATTAGAAGATCATACATATGTGAGCCCTTAGAT
AATGTTTCTGAGTGTGGCACTGTGGGAAGAAAGAACAAACTCATACCGCTAATACAAGGCAACCTAAATCTGACAG
AACCCCTGCCTTTTTCATTGTGAAAAAGTAAGAATAGAATCAATTTGCCATCAAATTGCTCATTGGCTTCCATAAG
CGAAGGTAATCTGTTGGAGTTTATTTATCGCTGGAAAGTAGATTGTGACAATAACCACGTCAGCCATGCTGAAGGC
AGCCCAAGCCTTGGGACCCACCCACAGTGCATCTCTTCCACCATAGATAACTGCTTTCAAGAGCCTGTTTCATGAG
CACTGGGATGGTTAAGGACAGAGGCTGGCTGATGTGTACCGAATGATTCATCCTGGACACCTGGTTGTTTAGACTA
CTTGTATAGAGGGTAATTTTTGCTGAGTAGTAACATCTGATACAAAAGATCCAAGCACTTTGTGTCTACTGTATGC
CTCTTTCCAGATATTCCTGTTCCTGATCTTCCAATCTTGCTCCTTCCAGGCCCCTGACCTGCTATTTAAGCCACTT
GCTGTTGCCTATAAGTCTGTGCATATTCTCACCTCAGGCCACTTCTGTCTACACAAAGTGGGTGAATCAGAGCCCC
ACCTGCCTATTGGCAGGACCCTCTTCAATGCTGTCTCTACTCATTCCTGAGTTGCCCACACTGCAGACACACTCTA
CTGCTGCCTCACACCAACACACTAAGCTGGCATGTTTATGAAGAAGCTCAGAATTTTCCTCTTAAGTCAGCTGGTC
ATAGAAACTACCCATAAGGTCATTGGTGAGAGTCGAGGTCAAGGCAAAGGTCCAGCAGTGCGAGGTGACATGGGGT
CTGAGCAAGGTCTTTACGTAATTAACACCAGTACTCAGAACATACGTACTTTAACCCAGGGGTCAGCCAATGGTTT
CAATAAAGGCCCAGATGGTTTCCCTTTGCTGTGTGTGTGTTCATTCCCCTCTCCGAAGCTATGTGTAACCCCATAG
AGATCACAGGGGTTCCCATCAACCACAGCCAGTTACATCCACAAAAACCTTGATCCTTGTTAGCAGATGGGAGAGG
GTGGCTTGGAGGATTGAAATCCCTACTTCATGGGACTCTGGATACTTACACAGTTGCTTCCTTCTGTGTGTCTACA
TGAATCTCAGCACTGTGTTTGGGACACCATCTACACTCTTATTCTTCATGCAATTCTGTTTGCTCCTCAGATGGCC
GCTTTTCCTTTGAAGCTCAACAGTAGCCATCACGTACTTGGGCTGAGGGTTACTGATTAACTCTGTGGGATTCTGG
GATCAGTTCCTTCCCTTTCTTAGCCATCCCTGTGCTTTACAAAACTTAGCTTCACAATTATACTCATCTCTCCCTG
TGTGCACCTCTAAGAAATTACTGATAAAACACGAGTTTTTTTGAAATTACAAAAATATATGCAAGAAAATGTTTAT
GGGGCCAATTGATCAGCACGGAAAAGCATAAACTTTCCTGTTTTCTGCCTCTCATTAGAATTTAATCCCCCAAAAT
TGAACTCATTCCAAACTTCTGGCTTAACTAGGAGTTGCTGTAGCCAGCCTATTCTAGACAGCATCCTGTAAGCCCC
TGGGATTGGCAGACACAAGACACTTTTTCCATTCATATATGTGGTTCTGTTCTGGCCAGATCAGTGGACTTCCAGT
GCCCTTCTTGAGTCATACTGAGGTAAATTACTCAGACCACGAACTCACATTTTTAAATAAATAAAATAGCAGCATA
GTTAAGAATTTATTTATCCCATTACTGGGTATATATGCAAAGGATTATAAATCATTCTACTATAAAGACACATGCA
CACATATGTTCACTGCAGCACTATTTACAATAGCAAAGACTTGGAACCAATCCAAATGCCCATCAATGATAGACTG
GACAAAGAAAATGTGGCACATACACACCATGGAATGTTATGCAGCCATAAAAAAGAATGAGTTAATGTCCTTTGCA
GAGACATGGATGAAGCTGGAAACAATCGTTCTCAGCAAACTAACACAGAAACAGAAAACCAAACACCGCATGTTCT
CACTTATAAGCAGGAGTTGAACAATGAGAACATGTGGACACAGGGAGGGAAACATCACATACCGGGGTCTGTCAGG
GGATGGGGGCAAGGGGTGGGATAGCATTAGGAGAAATACCCAATGCATGCGGGGCTTAAAACCTAGATGACGGGTT
GATGGATGCAGCAAACCACCATGGCACATGTGTACCTATGTAATGAACCTGTACATTCTGCACATATATCCCAGAA
CTTAAAAGTATAATTTAAAAAAAAGAATTTATTTCATTTGTATAGTCCATGAAATTTTTTGGACATTTTTTAAACC
TGCACTTTTCATAGTTTTTGATACATCCTTGCTCCAATAAATAATTTGCTAAAATTACTAAATCTCATAGATATAC
CAATGGTTTGCATCAGCTAATGGCATATTTGCTTGTCAAACTGAGGTCAGCCAGTCCCCCTTCATTCACTGTTAGT
AACTCATGTGTTGCATTGTTAAAAACCCAGTATCACCTCATCTCCCCTCTAGCATCTTCTCTGCAGAGGAGAAGCC
AGGCACTATATCTCCCAGGATCCCCTTCCCTGGATAGTTCCAGTTTACATTTTCCAGTGAGAGAGACTTGCATAAG
AAATGGGGCCCTGGAGAGATGGCGGGACAGACCTGTACCCATCAGTGGTGGCTGCAGGTGGAGGAGCAGGCAGATG
TCAGGTTCTCAGTGGCTTCCCTGCTAGGCTAGAGACCCACCTGCTTTGCTTGTGCGGACTAAGATGAGTGATAAGA
GCTTCCTCATAGGTTCTGGAGAATATAGCAATCTCGCAGCAGGATTCTGAGAACCTCCCATCCATGCTTCAAGCTG
AAGTCTTCAGTATATGCTTCCCTGACCTCCCTACTGCAGCTTCCAGGAGCTCCAATGGTGACTGGTATTATTATAG
TATCCCACTGCTGCTATAACAAACTACCACAACTATGTGGCTTAAAACAACACCAATGTATTCTCTTATAGTTCTG
GAGGTAAGAAGTCAAAAATGGGTCACTATGGGCTAAAATTAACATGTCGTCAGAGCTGCATTTCTTCTGAGGCTCT
AAAGGAAAATCTGTTTCCTCGCCCTTTCTAGCTTCTTGGGGCTGCCCGCATTCTTTGGCTTGTGGTCTCTTCTTCC
ATCTTCCAAGTCAGCAGTCACATCACTTTGACCTCTGATTCTGTTATCACAGCTCTAACTCTGATGCTGCTTTCTA
CTCCTTTCACTTATAAGGACTATTGTGATTACATCGAACCTGCCCAGATAATCCAGGATAATCTTCCCATCTCAAG
AGCCTCAGCTTAATCACATCTGAAAATTTCCTTTTGCCCTATTAGGAGACATTTTTATAAGTTCCAGGGATTAGGA
CACAGACATATTTAGGAGACCGTTATTCTGCCTGTCACATGACTTCACCAATATTTGCTACCCGTGGTTTCCAAAC
ATTAGTGTATGCTCTTATTTCTATTTATTCCTGAAATACCAGAGTGAGTCTGTTTTTCTGAAAACAGCTCACTAAT
GCCCAGAGGAGCTCAAAAATTTTCTGTCTGTTGTCTCAAAGTCTCCAAATTGTACTGATTCTTCAATGTGCATGGC
TGTAAATTAGGCAATAATCTTGCTGGGGTGTTGGAAGGAGAAGAAAGGGAATGATTAGACAGCCCTTCTGATTCGG
GACAGAATCTGATTCGGGACACTGTCCCTGATGGCTGGCCATCTTGGGCTGTAATCCAGTTTCCCTCCCACGTAGA
CCCGGTCATTTCTGACTCCCCAAGAGGTTTTCACAGTATTCATGTCCACTTCCTTCATCAGTCATCTTCTTGTCTT
ATATCTCCATGTTTACATTTTTCTTCTCTTCTACCACTGCAGTTCTGCCTTGATTCTTCAGCCATTGCCTTCCTTT
TGCATGCATGTGCTTCAGCCAGTTCTCTAGGAAGACATTCCAGACAGCTCATTATCACTTGTTACTATGTTTATTT
GACTGCAGAGCTTTCAAGATTTGTAACTTTCTATTTTTTAAATTTTAGTGTAAAAATAATTCTTGCTCACAGTAGG
AAATGAAACAATCAAAATATGCATAAATATACTTTTAAATCTCTCCTTTCACTAAATCCAAACCCACTTAACTAAG
GTGATTACTATTGTATCAGCCTACTCTCTGCATGTCTACTTTCTTCACTTGCACGCAAACCATAGTGAGTGTTTTT
ATCTGGTAGCTTTTGTTGAGCTGTAATGACTATAAGTTTTATATGTAATTTAATCATTTTCAGGGCAACTTGAGAT
ATAATTTAAATAGTATAAAATCCGCACATTCAAAATATACAATTCAGTGGCTTTTAGAATATTCACAGTTGTGCCT
CCATTACCACAATCAATTACAGATCCTTTTATCTCCTCCCAGCCCTGCAGAAAAATCCATATCCAAACCCCAGCCC
TAAGAAACTACCAACCTATGTTTTAACTCTACAGATTTACCTATTCTGAACATTTTATGTAATGAAATTATACAAC
ATGTGGCTTTTTGTGTCTGTCTTCTTTTACTTACCAGAACATTTTCAAAGTTTGTGTGGGTCGTAGCTTGTATCAG
TACTTCACATCTTTTTCATCGCTGAATTACATTCCATTGTATGGATATATCACAATTTATTTATTAGTTGGTGACA
TTTTGGTCCTTCTCATTTGGATTGTGATTAATAATGCTGCTATAAACATGGCATATAGGTTTTTATGTGAACTCAT
GTTTTCATTTCTCTTGGGTATACTGTATCATAGGAGGGGATTGCTGGGTCATGTAATAACTTAATGTTCACAAATT
TGAGCTACTGCTAGCCTGCTTTCTAAAGGGGCTATGTCTCTTAAATCCCCACCAGCAAAGTGTGAGTATTCTAGTA
TCTCCACATTCTCATCAACACTAATTATTATCTTTTTAATTATAGCCATTCTAGTGGGTGGGAAATAGTATCTCAT
TTTTGGGTTGATGTGCATTTCCCTGATGGTTAATGATGTTGAACATGTTTCTATGTGCTTATGGGCCTTTTGTATA
TTATTTGTCTTATTATTATTATTATTATTATTATTATTATTTATTGTTTATTATTATCTTATTATTAATATTATTA
TATTGAGTTACAACCATTCTTTATATATTCTAGATACAAGTCCTTTCCTTGCCAGATATAAGATTTGCAAAACTTT
TCTCCCATTCAATGAGTTTCTTTTCACTTTCTTGATGGTTTCCTTAGACTCACACATTTTTAATTTTGATGATGTT
CAATTTATCTTTTATTTCCTCTTTTTGCTTGTGCCTTTGGTATCATTTAAGATCAGGTATTTTTACCTAATCCAAA
GTCATGAAGATTTATGCTTATGTTTTCTTCTAAGAGTTCTCTAATATTAAGTCTCACATTTAGGTCTTTGATACAC
TGAGGTAATTTTTATATGTGGTGTGAAGTAGGGATCCAGCTTTATTCTTTTGCATAGGGATAGACTGTTGTTTCTG
CACTGTTTGATGAAAAGATAATTTCTTCCCCTCACTGAATTATATTAGCACTGTTTTATAAAACCAATTAACCATA
TAAGTGAAGAGTATTGCTGGGCCCTCAATTTTCTTCTGACTTCATTCTTTTTAATGCTGTTGTAAGTTGGATTTTT
AAATAACATTTGTATTAATGCATTGATACTCTATAGAAGCACACTCAATTTTTGTATTTTGATCTTGTAGCCTACC
AATGTGCTAAACTAATTTAGTATTAGTGGTTTAGTAGTTGCTAAGCTTGTTTTTAGTGAATTCTTTCGTATTTCTA
TATAAGATTTTATCATCTGCAATTGAGATAGTTTTACCTCTTGCTTTTTAATCTAGATGACTTTTATTTCATCTTA
TTGCTCATTGTAGCTTTTGAAAAATTACATTGTATTCACAGTGCCCTACCATTTGATATTTTTATTTCTATTATAT
CATGAACATCCTTTGAATTTCACAGAAAAAATGGTTTAACTCATCATTTTAATGCTGTTCCATAGTATGTTATATG
CACCTACTCCCCTGGGGTAAATATTTAGGTGGTTTCATTTATTTGTCACCCACTCTACAGATGTCTATATGCACAT
ATTCTCCCAGCCATCCCAGAGTTTTCTGATTTCCCATAACGTCAGTGATTAAACTCCTGATCTGTCTGACTAGCTA
GACATGCTGACTGACTGACAGTTCCATTGCTACTTTGCATAATTTAAGATCAACAGCTACATTTGGATCTCAGATC
ATCTAGGCCCAGCTGCAGTTAGTCTTTCCTAGTCTCAGAGATTCCCTCTCTGCCTGTTAATTTCTCTATCCCTGAA
ACTGCCCTTGTACTCCAGCCCAAGCAGCTTCCTAGCCCAGTAGTCCTGGATCTGCCACTTGCTAACTAGAAGGTGT
CATCTGCAGAAGGTAAGTAAGGCATTTATCTCCATTCACTTTATATTTAGTTATCAAATCTCCATGTAGCTCAGGT
AGATCGAGCTGAAAACATTCTTGTGTTTAGTAATATAGCCATTGAGCACACCATCATTGCTCTTTTCATACCTTAG
TCCTACTCAAAGATTAAAGTGACAGCTTCCCAGTTATGATAAAAAAGTATAAAACATTCTGAGGGACCTGTAAACT
ATAAAGCTAAAATATCAGCTTCTGCATGCAACATATATGTCAAATTCTATGTCCAATACTCTTCTCATTTTTAGAA
TTAATTCTGCAAACTTGAAGATTGAGCTTTTTATGAACTGATTGGCATCTGCTATGCTCACCAGATTATTCCCACT
TGACTGAGAGGATGGTGGTAGCAAGAGCTCCTTCCTTTCAGAGGCTTTTAAAAATACTTATTGGGAAGAATGTACC
ACATAAGGCATTTAACTAATGAAACCACAGATATAACCACTGGCAAAAAAATCAAACAAACATTTCTTTAACTGCC
CTGTACTTGGTACATTCTCTTCCTCCTGCTAGGGAAGTATCTCCTCTCCCTTCACTTCACAGACTGGAATTTTCCA
AATTCCTAGTCTCAACAAGACAAACTGTATTTTGAAATCACTGTTTTCCCAGAATACCCGGAGTGTTACCATGACT
CCAGAAGATGCCTTCAACCAAGACTCATAATCTGACTGTAAGAAACCTTCAGATAAGGTATTGGGAACTTCTGTAA
AATTGTAGCTGGGCTGTGATTAGAGAATTTTCACAATATCAACGAATCTTACAAAAGAGCTGCTGATCCAACAGGT
CCTCAGAGATCCTGTGTGGGAACCAGAAGGTATTCTGTGAAAAACGACAATAAGAATAACCATAATAACAATAATG
GCTGCATTTAAAAGGCTCTTGCTATATTTCAGGCACTGGTTTTTGTACCTACATGGATTATTTTGTTCAATATTTA
TCACAACCATATGAGAAGGATGTTATTATCCACAATAAATAAATGAAATCTGTAAACTGTAACTTGTTCAAGAAAA
TTAGGTACAGAGATGTTAAGTTACTTGCCCAAAGTTCCATAATTTTGAAGATATGAGGCTAGAATTCAGTTCCAGG
CCATTTTACACTAGATCTACTAATTTAGCCACTGTATGATTGTACCAAGACTGACCTAAAGCTACCTCCCCTTCCC
CAACAGGTTCTTTTATGACAGTGAGTATAAAATACAGGGCTAATGACGTATTGGGTGCAGAGAGGGGTTGGAAAGA
ATGTGGTATTTCAGACTATGCTTACTATGCTTAAAAATCACAATTCCGTGGTGACTAGAGTTTATCTTAAACTTTC
CTCCCTGGCATTCCATTGCATCCAGTTGAGAGAGCACTCTAGCTCCCTACATCCCAGAACATGTAGATCTGTCCGT
GGTTCTGAAAGACACACTGGCAGGTCCCGCCAACTACATGAATGGCATTCATATTGTGGAAAACTGCCCACTAGTA
AAAATGTACAGTTGAATTTGTACATTCGTTACTTACAAGGTTGGAGAACGTGAAGTACTGGACAATCCAACAATAG
CTTGGAAAGATACCATAAAAAGACCCGCAGAATATTCTTTTATATGCATTATTCTGAAATGTACAAAACAAAACCA
GAATGGGACACATTATGAGTGAGAAGTTTATCTTGAAATTTTAATCAGAATATGAAGAAAAGAAATCCAACATTAG
ACCTGGAAAAAGATAAAATAATTTGTATTTCATTTAACAATATTTATAGATTCTCAATATATACACTATCCTTTTA
AACCTCAATTTATATTTAACCATCCATTTACCTTTATTTTTTGTTTGTTTTTTTCTTTCACCTCATACTTTCTACC
TGGGATATCTTTTTGTGACTAAAGTACATTCTTTAGAACTTCTTGTACTTTACTAGGTGTGTTTGTGTTAAATTGT
TTGTCTTCAAAATTCATTGAGATTTAAATTTTATTATTATATTTTCATGCTTAAGTGTTATGTGTTTGTCTCTTCA
AATTTTTCTGAAGATTATTTATAGTTTTATAGTCACTAAATGTATTTAACAATCTGTTCTTTAATCATAGCCCATT
TAGCAATTTTATATCCTATACAATTATTCTGATTTCTCAGTTATATGATTGTATGATTCTGCTGTCTGTTGTATTT
TCTTGTTCTTTCACGGTGCTTTGTCTCTTATGAACATTGTTCTCTGTTGTTGTTCTTAACTGTTAAGTGCTCCTTT
TGCTGGTGTAATGATTTTTTGGAATTTTCAGAGTGCCAATGACAGTAGAAATTTTCCAGAGAAGTTTTGCTTGATT
TTCCAAGGTTCCTTGGAGTCTGCACTACTTTAAAATAAATTCATGGCTTGATGATTTTCAGACCACCCAGGGAGTG
TGAACTCGGGCTGCAAATCCACTTCTCTTTAATCTCAGCAGAAGGTTTTGCCCCTCTCCACTCAGCACCAAGGTGA
CTTTCTAGGGAGACCTTCAAGCAAGGATGTTTTCCTTATACTGAGGAGCTGGCTTTGGGATCCTGGTGAAATGAGG
AGAACACTCTATTAAATTTCTCACTCTGAGTGCTACCTGAACTCTGTCTTCTATTCCAAATGCCTTAGGAGGCCAT
GAAAACTAAACCTTAATTATCCTACATTAAGTAAATAAGCTCAGCAAAAAAAGTAAATGCACAATTCCGTGATTTA
GGATTTGACAATTTTTATTAAAGTTTAAGCCTCTCAATGCTTCTACATTGTGTTTTATTTTTTCACCATCCATTTT
TGTTAGCATGAGGGTAGGTATGTTTACCTAAATGTGCCATGTGAGTGGTTCAAATCTCCTCCCCAACGCTCCTTAT
CACAAACCTTACCATCTTATTCCTTTAAAGTCACTGACTATCTAAGCAAACTACTAGCTACTGCTGTGCATTGATT
AGGACCCAACAGTAAGACGTTTCTCCTTTTGGTAAACTGCACTTCCAGATGGTCTAATTGAACATCTTCTCACTGG
AAGTATTTTTATTTCCATTACTCTTAATGATGATTCCTGAAAGAGACTCTAATGCCCTGACAGTTAACTCTGGGGT
ATCCAAGCATACTCAAATTTTTTTCCTTCTACAGTCAATTGTCCTTAGGGAACTCCTCAAGACCTCAATAATGAGA
CAAGGGAGAGTGGACTCTCCAGTGGTAATAAGAACATTAGGAGTATAGTACAAATTATTAGGCAACTTACCTGGCC
TTATGGAACCTGCTAAAGTCTGATGCTGTGTATACCATTTCCCCTTGCAGACTGAGGGCTGGTGAGCATGAGTGAC
CTGATAAAACTCAGGTCATAATGGGCATCTCAAGTCACTTGGTCATTTTCTGTATCATGATCATAGAATTCAGAAT
CTACCAGGGCCCAGTAGTAAGCCAGAAGCTATTACTCAAATTCAGAAAAAGAATGGAACTTTTTCCCCAAACCCTG
AAACTTGGGCAATGAGTCCTATGACTGCCACACACAAACACACACACACACACACACACACACACACACACACACA
CATACACTCAACAATATCCAAATAAGCATTTAGGGCCAAATGGCAGATATGCTTGACTTTTGCCTGGATCAAAATG
ACATTTCCATTTATTATGGAATGTTTCTGTACTTGTACTAGCTCATAGGATTTTGGATTAGAGAAATACCCTTGCA
GGATATGAAGTCATACTTTTTCATTGGTGAAATCATCTGTTCTCATTAGAGTTTAATAATATCATGCAGTTATTTC
TCAAAGAGCTTGTACTCCTTAGATTTGCTGGAATTCCTCAAGTTAAAAAGACCAGGGCCTCAATAATGGGAGGTCT
CATTGCCAATAGCTACAACTGGCTGTAGAATTTATTAAAAACATGTGAGTGCATTGTTTGGGGAAAGAATCAAGAA
CAAAAGCAGTGCTTGTTTTGTACTTCACTGAACTACTTCCATGGCCTCCATTCAACTTTAGCTGCAGTTTTGGTGA
CTTGATTCACAGACTGTCGGAAAGTTATGAGCTATGGAATGTGCTCTCTCCAATACTAAAGAGGCCAGTCATATAT
ATGTAGGGTGTCCTTCTTTATCATTGTAGGTTGTATGAAGTCAGTGATTCCCATAGAACCTTGGGGTCACAGATGC
CAGGCCTAATCTTCTTTTATTATTCATTCAATCTTTATTTTTTCAAACAATGGTCTCACTCTCTTGCCCAGGCTGG
AGTCCAATGGTGCGAACACAACTCACTGCAGCCTCGACCGCCCCAGGCTTAGGTGATCCTCCCACCTCAGCCTTCC
AAGTAGCCGGGACTACAGCTGTGTGCCACCGCACCTGGCTAATTTTTGTGTTTTTTGTAGAGATGGGTTTTTGTCA
TGTTACCCAGGCTTGTCTTGAACTCCTGGGCTCAAGCAATCCACCCACCTTGGCCTCCCAAAGTTGTGGGATTACA
GACATGAGCCACTGCACCCAGCTCTAATCTTATTTTAATTTTAGAAATCTAACTTGACAGTTTCTGGTGCCTGGAT
CCATATTTGTCTCAGAAGATTGAATTGTGGACTGTGACCTTCTCTCCCAAAAGTCTCTTTCAGGGTAGAACTTCTG
GTTATGGCAACATGAAGAGATAGACCAGCAAGTTTTCCTCCAACAGCAACATGAAAACTGTACTAAATACTAAAAA
CAAACATTTGAAGTCACTGGAAAATTAACAAAGGCAAGCAATATGTGATAAGAGCTTTCTTGTTTAAGACTCCTTC
TGTCTTAGGTAAGGACTGCAAGTTGGTGGCCTTTCCTTCTTACCTATGAGTGCTCTTCAGCCTCCCAGCAGATAAC
TGCAGCCCTAGTGGCTCAATATGGGCAGTTTATAGAGTTCAGAGCCCAAATCAGCTAGAAATGTAAATGAGAATGT
TGAAAGAGAGCTGCAGAAGAGTTGAGATCAACAATGTGAATGCAAATTATGCTCATCCCTAGTTGAACACTAAACT
CTGCATGGGTGTGGGCCACTTGGGAATCTGGTGAAAAACCAGAAAGAAACTAGAGGGAGGTCTACCCTCCAAAGAG
ATAAATGATCCTGGTGAGGTCTGCAAGTCTTCAGTAACATGGACTGAGTGCATTTGCCAACCTGCACAGAGCTCAG
TAGAAGAAAGCAAAAATCTTACTGGCTTGAGAGGTGAAAAGGCAGGATTTGAGGCTAACAAAACAATTGGAAATTC
AAAGAGAATTCCAGAAACAAAGCAACCACAATGAAGTTGAATCACAAAATCTGAGTATAAATGACCCAAATACTTG
GTCAACCTCTAAATTACACAGATGCAGGACACACACCCAGAAACCCCTGCTGAAACCACGTAAGATACTAAGTGGA
AATTCTCAAACAGAAAAAAAAGAAAATGAAAGTATTCTGGAAGAGGTCCACAGAAGTTTTGAGATGGCAGAATAAA
GAACAAGCCTGTGCCTTTGAATATAAATCTCCAGAATGTGCTCAATTCTAAAATCCAAAACACAGATTGAAATAAG
ATTAAAGAAAAAAATGAAGAGAACGTCATGAACCCATCAGAGAATATAAAGCAGGTGACCAGATATGCAAGTGGAT
CCTCGAAAGAAGACACAGGATCAGAAAAAAATATTTGAGAAAATAATAGTTGAAAGTTTCCCACATTTGGTGGGGG
ATTTTAGCTCATATCTAAGGTGTTCAATAAATGAAAGCAAAATAACTGCAGAGAAAATTACAAATAGGGTGACCAA
GGCCTTGGCCTGGCCTGAGGCAACTTAGCTAGCCCCAGGGATGTGTGTTCTGGTGTGGAGTAAGATCAGGGAGCTA
CCAGTGCAATCTCTGCCTCTACATCAGCGGGACTGATCATTTTAATTTTGATTGCAGTTACTGCTGGTATCTGAAT
CAAAGTGGTGATGCTTCCTGAGGAAATTTCTGAAGCCATTAGTCAATTAGAACATTATAAGCCATGCCTCCCGTGA
AGAGGAGCATCTAATCCCAGCATTTGCACCAGGGAACTCTGCCTGAAGGGATTACCAGTGACTTGAAAGTGTAACC
AACAACAATCTTGTCACTATTACGCACCAGCTAAGCAATCTGAACATACATGCTGAAGACATATTTGGTGAGCTAT
TTAAGGAGGCTAACTACTTCCACATTAGATCAAATTCTCTTAGAATTTGTTGCCTCATGGTCAAAGTCACTCAGCT
GGATTCAGCAGTGGAAGAGGTGTCACCACAGGACATCAACATGGAAAAAGCCCTCAAAAGTTCCCCAGCCCAAGAC
CAGCAAGCAGTTTCAAAGAGCAGCATTCCTACTCCTGCTGCTGACATTTACAATCAGAGTGATTGACCACCTCCCC
TGAATAGCATGAGAGCACATGGAGATGATAAAGACAGGCTGAAGTTCTATACTGATCCTTCCCATTTATTTGACCT
CTGGAAAGAAGAAGTGCTGCAGGACACAGAAGACAAAAGGAAAGACAGAAGGCATCAAAAGTAGCAAAACCACATA
GATGGCACCACTTATGATGTGAAAAGGATTACAAAAGCCAGAAACAAGCACCATGAGTGGAATATGATGGCATATG
ACAAAGAGCTTAGACCCAGCAACAGGTTGTCTCAGTGTGTACCATGGAGCATCCTGCAAGAACCCCTGTCCCTAGA
CAGCAGGTCCCATACAATGGATGTTAGGGATGCTCTTATCGAGCTACTCCAAACCATTCTCACGACCCTTCCTAGG
CAGCTGAAGATGCATGTCCACATGGGCCCACAAACCAGGCCCCTGAGCTTGAGTGCCACCCCTCCTCTGCCTCAGT
GAGGCACATGGCCCTAAATGGACCTCAAGAGCGGCCCCCACTTCCCTGCCTTACCCCAGCTGTGTTCCAATCCTGT
GCACTTATGGCTCCAGGAGACTACAGGATGCTCCCAGCACAGGTGCTTGCATATTACAACCCGTCAAGACCTCCTT
CTCTTCCCCCTTTTACAGTACAAACTGCCTTTGTTGCCTTCTGCAAATCCTCCTGCCGCCTCCTTCCTCATATCAG
CTGGCTCCACACACCCTCCTCCTCACCCTCCCTCCACTGGCCCCTGGGTCCAGCATTCCCTCCTCCAGCCACACAC
TTTCCCCACCAGGCCTCTTGGCTCAGTTCTTCTTTTTTGTCTTCCCCATGCATTCGCCCCCACACTGCCACCAGTA
GCTGAGGCAAAGTGTCAAGAACCTGCACAGCCACCAAAAAGTGATACGAGAAGTGATCTCCTTGCTGCTACTGGAA
TGGGGATTTGACTGGAAAAGGTGCAGGATCTGTGTGAGCAAGAGGCCAAGAGTGAGCCCCTTGGGAAGGACAAGGT
CACAACCCTGTCAATACATTGCAGTGGAGAAGTACAGGGATTCTGAGGGAGACTTAGAGTTTGACAAAAACAACTG
GCCCAACCAAGGCAGTGCTGGGGCTAGCTCTATGCAACAGGTGGGTAGCTGGTACCAGGCTGGGTGACAGCCAGGA
GACCATATGCTAAGAATGTACTTAAGATTTCAAGTGGTCTCAGCACTCAATGGTATTATAATCCCGCAGCTGAGCA
CCTTTTGTATTAATGATGTGATATTGCTTCTGCACATCCAAAAATTCTGGGCTTTCTCAGTATTTACTGTGTAACA
TTTAAATGCCATTAAGCATAGAATATCATGCTGCACATGAAGAAATAGGATGTAACTATTACATCGTCCTGAAAAG
AGCGTTTTTTTTTTTCCTGTAGGCCATGAAAGTATTTAGTGTATTTTGGGTTTACTCTTATTGATCATTACAATTC
TGCAGATGTGCTGTGTGAAGCTGCCTGGTGCTAGGCCTTTGAAGATTAATAACTGGGTTTCAGGTGGATATCTTCA
GATGAGTAATTGATATTGTTTTACTGTTTTGGGGTGTTTTATGAGTTTGAAAAGCTGTTGGTTCTAGAGGGAAAGG
TTTAACAAATAAGGAGAATCCAATATTTCTGCTTTCATTTTCTTGGCGTGGTATCCTCTGTGTGAATTTGAATGCT
CAGTTGAATAGTTTTATTGCCTTAGTACACACTTGTCAAATGTAAAGTTGGAAGGACCCTTTTCAGGAAGGGTTTC
CTGTGAACATTCACTTAGTAAATGCTTGTGGATGATTCTTGTAAGAGGTCTCAGTGCTTAGAAGTAATGAAAATGA
AGAACAAGATCCTTTCACCTTCAGGGGACTTTGGGACCAATACTCAAAATATTTGCCAGCCCTTACAGCTTGGGCA
CCAGCCAGCAGGAGCAGAGGTGAGTGGTCCAGGAGCTGGGCAGCTCTGGGCAGAGCAGCTGCATGTGGGCCCTGGG
TGCTAAAGCAGGCCTGCTCATTGTGTGTCCTGGAAGAGCTGTGTTCTAAAGGAGGCCACAGGAGTTCTTAGCTCCT
TAGTGTGTGACAGACAGTGCTTCAGAGGGCACTGTGCTCCACTTGGTCTTAATTTTGAAAGTTTACATTGAGGTGA
AAGTAGATATTGACAGCTGTTCACTCTTCATGTGCTGTGTTCAGATTACGGATGAATCCATGAGCAGGGCCTTTGC
CTTATCCTAGAAACACGTGGTTATCTTCCCTCATGTTGTTGCTGGCACATCACATGTTGATAAGTACCTCTCAAGA
TTACCTTGATAGACCCGTAGCTGTAGAAGAGCACTTAGTTCTCACCTCTGCCCTTCCCCTTATTCTTGCACCGTGG
TCATGCTAGAGACCCATGGGGTTTATTTTCCTGGCTAAGGCAAACTGTTGGGAAATGCAAGAATGAGAGCTTCTAT
TGTGTAATTCTGGATGAAGAATAACTGCTTGTTTAGAACAAATAAATGTCCCAGGCTTTGATTATAGTTATGACTT
CAAAAATATGCTAAGACCATTTACTTACTGAGGAAATCTTACCTACTTATAGTATTTCAAATTGATTGCAGTCATT
AGTGACTTTCAGTTACGATGAAGGGAAGAATCTTGAGCTAAACGCAGGGTTGTCTGAGGTGATGATAGGAGTGGAA
ACAGGATGGTAACTAATGGATTTAGGTTAATGAGTTCTGAGAAAATCAACCAGCCCCTGCAAGTCACAGCTCTGTG
TCTCTAAGTGGACACCTGTCTGTCTTGAGGCAGTCATCATATAATACGCATCACATAGATTTTAGTCATATGGATT
TTGAAACTCTGGAAACCATATGTCTGCAGCTCCCTTTCATGACCATGCTTGTGCTGGTTTATCTGATCTCCTCAGC
TGTACCAGCTGCTGTGGCATGAGCTTCTTGAGCTTCTCTTTTTAGAGCTGATCATTATCTGGCGGCTGTCGCTATA
ATTTTCTTGAATACAAGTAAACATTATTTATAAACTTTTAAATTTCTCTGTTTAATTTTTCTCTCTCTTTTTTTTT
TAATGTAGAGGCCTACTTTGCTGGCATCTTTGGGTCCTACTCAGTCATAAACCACCCTCCCCACCCTAGAGTAGCC
ACTCTTGATTTCTAATACAGATGAGTTCTGCCAGGTCTAGTTGAATGGCATTATACAGTCACCATCATGACCCTGG
GTCTCATCATCTCGCTGTGTGTGGTCGTAGCCTGTTCACTCCCATAGCTGTCCAATGTTCCACTTTATAAATAACT
CACATTTCTTGTCCTTTCACTGTTGATAGACAATTTTGTTGTATACAGATTTTACAAATCGTGCTGCTATGAATAA
TATTTTCATATAGTTTGGCACACACATACATGCATTTCTATTGGTTTAGAACTAAGAGTGCTATTGCTGGATCATA
GGTGAGTAGATGATTGGCTTAGTCTCTCAAAAACCAAGTTTCTAGTAGTAAACAAACAGTAGAGGAGGGTGAATCT
CTTACTTAGAAATTATTCAGATAATAAATGAAGAAGGAATGAGAGACTGAGACTATAATAACTCTTTTGCAACCCA
TCATGAATTAACAGATTTAGCCATTGAATAGCAATGGCAATTAACAACACAATAAAAGAAAAAACTAATGTGAGGT
TCTTCCTCTTGATGGAAGAACACAATACAGCACCATCAAACAAGTCCAAGTGAAAAAGAAACCAAGCCTGAATAGA
AGCTGATCTCCATAGCCAATTACCAATTTAGAGGCAATACAGATAATAGAAATACATATTAAACTATGCCTTGGGG
AGCAATCAGCAAAATGCACACTATGGGAAGCTCTACCACACAATATAAATTTCAAGGAAGAATCTATAGAATAAAG
ACAACAAAAGCATATTTCTAAAGGTAAAACTAAATCATAATATTGGATGATAAAAATAAAAATAAAACAAAGAAGT
GATGATCGTAAAAGTCAGGTTGTGTTTTTATTTGAGGGAAGAAAGAGTTTATTGCTGGGCTGGAGCAAATGATGGG
GCTTCTGGTTTGCCCCACAAACATCTATCCCTGGTGATTAATGGGTGTTTACTTTGTATTAAAGGACACAAATTTT
AGACATTTTTTTCTTTTGGAGTCCTATATTTTATTTTATGACACAAAGGCTAATGAAAAAATAATTTATAGAGTAT
GGTTACTGTTTTGCACAAAGCCTCCTCCCCATCTTCTTTTCTGGACACTGAGCACCCAGAACAACCAGCAGCCCCA
GGACCCCCAGCAGGGGTGACCTCACTTCTAGGTGTGCATGCAGCTTACATCACCAGAGGTAACATGCAGGTCTACT
CCTTGACTCCTGGAAGAACCTGATAGGACACAGCTGAAGGGAAGCCTTCTCCGCCATCTGTAGGCTCTTGGCCATC
AGTGTAGAGGGAGCGGGTCCTCACTTCTCCACAGGCGTCTTCATGGCCATAGCCTCCTCTCTGCATGGGGAGTAAG
GCCTGGCCCTTCGCCTGAACACGGTGACAGGGATCTCGCCAAAGGTGGAGATCACACCATTCCTCCTTTAGCAACC
TCACGCTTCTTTTGCTTTACAGGAAAGTTGACTCAGGCTGGTGTCCAGTAAAGAAATCCCAAGGAGAGGCCGGGTG
CGGTGGCTCACGCCTGTAATCCCAGGACTTTGGGAGGTCGAGGCGGGCGGATCACGAGGTCAGGAGATTGAGACCA
TCCTGGCTAACACGGTGAAACCCCGTCTCTACTAAAAAATACAAAACATTAGCAGGGCGTGGTGGCGGGCACCTGT
AGTCCCAGCTACTCGGAAGGCTGAGCCAGAATGGCGTGAACCCGGGAGGCGGAGCTTGCAGTGCTCCTGCTCCCAG
GAGGCGGATGCCCAGGATGAACTTTAAGGTGGAGAGAGGAATGTCATTGCTCATCCTCCAGGTTCCAAGTAAGAAC
CTTCCTGCCCAGCCCATCTCGATCTCCCTGAATCCTCAACGCTAATAAGGACTGTCTCCTTCTCACCTCCCTAGAC
TGGGCTTTTTAACACTGGAGAGTGGATGTGATTTCTAGTTTCAGCATGTCCTGGTTTATTGTGCTGCCAGTAAAAT
AAAATCAAAATACACATTGAATAAATAATAAATAACCCATAGTGAGCAAAGGCTTACAATGTATTTTTTTGAGTCA
AGACCTCACTCTGGCCTGGGCTGGAGCCCAGTGGTGGGATCGTAGCTCAATGCAGCCTTGTATTTATGAGCTCAAG
GGATCTTCCCACCTCAGCCTCCACAATACCTGGAAATACAAGCGCCCACCACCAGGCCCAGCTGTTTACTTATTTT
TATTTTTTTAGAGACGAGGGTCTCACTATGTTGTCCAAGCTGGTCTCTAATTCCTGATCTCAAGCAATCCTCTTGC
CTTAGCATCCTGAATTGCTGGGATTACAGGCATGAGCTGTCATGCCCAGCTTGTAATGTACTGACACTATCAAGGT
TTCTTTCTCCAAGTGTGGACACAGCAGCAGACACCCCTTGTCTCTTGGGTCAGGACACTGGGTAGAGTGGAAGAGC
AGGGCAACAAAGTCACTGCAGAAAGCACCCACGTGGAAGAGGTCCAGCAGGGAGAGCCACCTGTTCCAGGGACACC
ATATTTAGGGATAACTCCTCTTTCTGGGCAGGACTGTTCTTTGATTACTTTTGTATTCACAATAGTTCTGAAATCG
TAGGATGATGAGACTCAAGACTGGCTAGGGTTTTATTTTGGTTTAGTTTTTATTTTGTTTTGTTTTGTTTTTTTCA
AAAAACCATCATGAGATTTGTTAATCTTTTGAATGGTTTTTGTGTCTCAATTTCCTTCAGAACTTAACTCTTCTTA
ACATTTCACAGTCCACTTTCAGTTAAAAATGTACTAGTACCCATAAATAGAGAAATCTTGCAACCATTTATCCTCT
ATAAAATGTCCATCTTTATGGTACAGATATTATATGTAATATATCTATATAGGTTATAAGTCCCACAAGAAAAAAA
CTTCTTCTTATCTTTAAACAGTTCTATGTATATAAATTAGAGTAAATGAGGGGAACAAGATAGCCTTTTGCACTTA
GCTCAGTATTTACCATTTTTGATCATCTTCATCTTTTCTGATAACCTCATTTCCCACCTGGTATCATTTGCCTTTA
GCCTGAAGAATTTTCTTTACCAATTGTTATTGTGCAGATCTGCTGGTGACAAATTCTCTTAATTTTCTTTTACCTG
AAACATCTCCTTATTTTGCCTGTCCTTGAAGGACATCTGTGCTGGATATAGAATTCTTAGTTGTCCTTTTTTTCTT
CCAGCACTTTAAAGGTGTTATTCTACTTTCTTTTGTTTCTATGGTTTCTAATAAGAAGTCATTGGTCAGGACAAGA
GGAACAGAGAAACCAAAACTGAGGAAATAACATGGTAGACCTAAAACTTATCAATAATGAAAAAAAAACCTATATG
AATCAACACTCCAATGAAGGGCAGAGATTGTGTGAATTGGTTAAAAAAAAAAACAAAACACAGAACCCAACCATAT
GCTCTTTCCAGATATGCAGTTTCTTTGTTTCTTTTTTTTTTAATTTAACTTTTATTTTTAGTTAAGGAGTACATGT
ACAGGTTTGTTATATAGGTAAACTTGTGTCATGCGGTTTTATTGTACAGATTATTTCTGTACCCATTAGTACCCAC
CCAAGTATTAAGCCTTAGTACCCATTAGCTATTTTTCCTGATCCTCTCCCTCCTCCTCTACCCTCAAGTGGACCCC
AGTGTGTGTTGTTCCCTTCTGCGTGTCCATGTGTTCTCATCATATAACTCCTACTTATAAGTGAGAACATGTGGTA
ATTGGTTTTCTGTTCCTGCCTCAGTTTGCTAAGGATAATGGCCTCCAACTCCATCCATGTTCCTGCAAAGGACATG
ATCTCATTCTTTTTTATGGCTGCATGGTATTCTGTGGTGTATATGTGCCACATTTTCTTTTTCTTTTTTTTTTAAT
GGAAAATAACTTTTATTGAGATCCCACCAGTTGCAATATCTGTTCCCAGCATTAAGCTCCTTCTTCCTTTGCAATT
CGGTCTTTCTTGAGTGGTCCCATGAATGCTTTCTTCTCCTCCGTGGTCTGGAAGCGGCCATGGCCAAACTTGGAGG
GGGTGTCAATGAACTTAAGGTCAATCTTCTCCAGAGCCCGCTGCTTTGTCTGCACCAGCAAGGACTTGTGGAGGGT
GAGCACCCACTTCTTGGTTCCCACCACACAGCCTTTCAGCATGACAAAGTCATTGGTCACTTCACCATAGTGGACG
AAGCCACCCAAAGGGTTGATGCTCTTGTCAGACAGGTCATAGTCAGTGGAGGCATTGTTCTTGATCAGCTTGCCAT
CCTTGATAAGGTAGCCCTAGCCAATCCTATAGATCTTGTTGATCTCAGTGCGGTGATGGTAGCCTTTCTGCCCACC
ACGTACCACAGAGAAGCCCACACGAGCAGGATGCCATGCCCCAATACAGGCCACCTTGCACAGGCCTTGGTGGGTC
TTGCGGGGCAGCTTCTTGGTGTGCCAACGACTGGTGACCCTTTTGTAGCCTTTGCCCTTGGTCACCCTGATGACGT
CGATCATCTCATCCTGCCCAAACACTTGGTTCACAGGTACCTGGTGCTTGAGCCTCTCGCCAGCCCAGTCCAGCTT
CTCAGCCACAGTGCCTCCATTCACCTGGATCTCCATCAGGTGGGCCTTCTTCTGGCACAGAGGAAGCAGGTGCATC
TGGGTGTGGGCAATGACGCAGATGACTTGGCAGTACTTCTTCATGCTGCTGAAGTCCTTCTCCAGCTGCTTCTTGC
CATCCTCATCCTGCCATTTCTTGCAGTACTTGGTAAAGGCCTTCTTCTTAGATTTATGCCAGTTCTTATAGAAACG
CCTCTTGCATTCATCACTGATGTGCTCAGCGAAGACAGTCTTGCAAGTCCGGAAGCCTTGAGGGGTTTCCACGCAG
CCCACAATGCCCACTGGTGGCCTCTCCACAATGGTCACAGCCTCCACCACCTCCTTCTTGTTCACCTTGGATCCTG
GCCTGTCGACTTCCCGCACGATGTGGGTCATGCCAGCCTTGTATCCCAGGAAGGCTGTGAGGTGGACCGGCTTGGA
TGGGTCATCCTTAGGGAAGCTCTTCACCTTCCCACGATGCCTGCTGCTGCGCTTCCAAGGCAGGAAGCCGAGGGAC
CCATGTCTGGGAGTGGAGAACTTTCTGTGAGACATCACGCCATCAAATCCTGCCGGTAGAGCATATTTGCCACATT
TTCTTTATCCAGTCTACCACTGATGGACATTTAGGTTGATTCCATGTCTTTGCTATTGTGAATAGTGCTGCAATGA
ACATATGCATACGTGTAAGACATGGGCTTTCTTTTTTTAATTTGCTTTGTTTTATTTTACTTTAGGTTCCAGGATA
CATGTGCAGAACCTGCAGGTTTGTTACATACGTATAGTCTGCCATGGTGGTTTGCTGCACCTATTGACCCATCCTC
TAAGTTCCCGTCCCTTGCCCCCCACACTCCCCAAAAGGCCCTGATATGTGTTGTTCCCCTCCCTGTGTCCATGTGT
TGTCATTGTTCAACTCTCACTTAGGAATGAGAACATGAGGTGCTTTGTTTTCTGTTCCCGTGTTAGTTTGCTGAGG
ATGATGACTTCCAGCTTCATCCATGTCCCTGCAAAGGACATGAACTCATTCCTTTTTCTGGCTGTGTACTATTCCA
TGGTGGTATGCACCACATTTTCTTTATCCATCTATCACTGATGGACATTTGGGTTGGTTCCAAGTCTTTGCTATTG
TAAATAGTGCTGCAGTAAACATACGTGTGCATGTGTCTTTATAGTAGAATGATTTATATTCCTTTGGGTAGATACC
CAAAGCGTCAAATAGTATTTCTGGTTCTCGATCTCTGAGGAATGGCCACACTGTCTTCCACAATGGTTGAACTAGT
TTACATTCCCAACAACAGTGTAAAAGTGTTCCTATTTCTCCACAGCCTCACCAGCATCTATTGTTTCTTGACTTTT
TAATAATTACCATTCTGACTGGCGGGAGATGGTATCTCATTGTGGCTTTGATTTACATTTCTCTAATGAGCAGTGA
TATTGAGCTTTTTTAATTTTTCTCATTAAGATTTTAATGTCTCATTTTAAAAAATTAAAGGAAGTTTCCATTTATT
TTTTAAGATAAAGATTTAGCGCACCCAAATGCCCCCAAAGCCAACAGAAAAATAGCTTTGCCCTGTCATTTCCCTA
AGAAAGCACTGCAGTTACTCAAAATATGCTCAAAGAAAAAAATGCAATCCTCTGAGTTCTAAGTTTCACGAAAGGA
CCACATGTTAAACTATGTATATCGATTTGATGTGTAAGTATGCAATAAATATGTACACATACATTCCTATCTGCTT
CACATCATTCTAGAGTATTCATAGTATATGAAGATGGGATTTAGAAATGTGAAAAGGCTGTAACAGTGAAAAGGAA
AAAAAGGGTACAATGGTTTTTAGACCAGTAGAATAATAATGCTTAGCTAGTTAATTATTTTAACTTTGGAGCAGAC
TAAAAAAGTTGTTTTGAAATAAATGGTACCTGTAAGTGATGCTTACTGCAATACTTGTTAAGTGTTATTTGTCACC
ACTGACAGGAATTATATAGTGCTAGTAACACAGCTCCCACTGTCCTAGAATTCCTCTAAATGGCCAGCTATTGCTA
CCATTATCTTGATAAGCATTTCAAGAATTGTGTTGCTGAGCCTTTTAAAAGATAACTAGTTTATAACCTATGTCTC
CTTATGTGCCTACATCAGTTACTCATGGTCCCAAAGAAGTATAAAAGAGGATATAAGCTGCTGAAGATTTCACAGA
AGAAACAGAGATATCAGAAACTTCATGATCATCAAACTTAAACCACCGCTGTTTTGCTGCATTTTTACAGTAGGCT
GTGTAATGGCCTCCATCCAGCCCACCGCAGTGATCTGAAACAGAAAACAAATTATATTTCTTCAAATTGTTCTTTG
GACCAATAACATACTGTGACAAGGCAAGATTTTCTAACAGGAAGTCCACAGATGTCTGTAATTTTTGTTTCCACCT
GCCATTGTAGGAAAAATGTTTCAGATGCACTAAAAGCACAGGTGGTAACTTCCAGATTTCTTTTTTAAGAATCCCG
TCGAGCTCTGCAAAGATTGCAGTAAAATCTGTTATTATCTATGAGTTTTTCTTCTTTAGAAAATAATCTAAGGCAA
TCCTATAATGTACATTTACTTGTGGATGCTATCAGTAGAGACAAATACATGAAGGCCTCAAGTGTCCTAGACTTTT
TGTGACGGGTGAGGCACTGTACTGTAGATTTGAATTGACCCTGAAAAAGTGCAACAATAATAGACTCATAGAGCCG
CTTGTGTTTCTGCCAGGCATGTTCTGCAGCTTTAAAGTCATTGAGATGATCATTATTTTCTTCTTTATATGTCTTC
CGATTATCAGTTTTATTTAGATCTTCATGGAGACCATCCATTAGGAACAGAAGAAATTCTTGTGAATCTTGACTGT
ATCCTGCAAACTGGTAATTGATCTTCCCAATGGTGACTTTAAGGTCTTTTGGACTGATATATCTATACTGTCCTGT
CCACGGGGCTTTCATGATTATACCAAATTCTTCTGCCACTTCACCTTTATGCCCCTAACAAATTTGACTTGTTAAT
ATCATCCTGATAACAGTTTCGGTTGAAATAATCAGCCAAATGTGGAGCATTACATAGGCACTGCAATATTGAGTTC
ATATAACAAGTATTTCCTAAGTTACGAAGTCCAGTAAGAGCTGGTCCAGAACCTCCAAAAACAGGATTGAGTTTCC
AAATCTGAGAAGCAGAAAGCCTTGAGATCTCAGCTTTAGGGTAGCATGTTGGCTTGTCTTCCTGATTAACTGTTGG
AGTTACTGCTGGCTTCCTCTTGAATAGCCTGGGTTATATCTGGGGAGGAGTAAGAGCATTTCAGTTTGGAAGGTTC
CCTATCCTGCTTAGCAGGAATCTGTGGCTTGCCTTTATGAGTTGGAGGGGTGGAAGGAGGTGCAGATGAAGGAGCC
ATTTCCAGTGGGTACATATGAACAGTGTTAGTGGTGAATGATAATAATGAAACGTTCCAGTGATTGGATCAAGAAA
CTTGGCCCAGCCTGAAGGCAGTCCTGGTACCATCCTCCCCATTTCTTCACTTCGTGCTATTATCGAAGGTTCTCGT
TGAGTTTTATTTCTTTCAGTATCATCTGTATCCTCTCTAAAAGTTTCTGTCCTTAGAATTCCAGTTTCTGGTTGTC
CTTTAATCTTAAAAGGCTTGCCTGAACCTGAATCCCCTGTCGCAGATGTATGGGACACATCTCTTGTTGACTTTTT
CTGTACTTCTGGGGTTGAACATCTTCTCCCCCTGTCCTCTACAGACTTCTCGGCACCAGAGGTTTCATGATCACTT
TTACTTTGTCTTTTTACTCTTGTTATTTCTTTGCCCTTCTTTGCTGAGATTTCTTTATCCTCTTTCTTGGCCTGTT
CACGTTCTTTCTTCTCCATTTCTTCTTTTGCTTTTTGTTGCTTCTGTGTAATTTCATTTTCTTCAGCTTCTTGTTT
CTTTTTGGCTTTTTGCTCGTGTTCTTCCCTCCTCAGTTTCTTTCTGTTTCCCTTGCTGTCTTTCCTGAAGTTCTTT
TTCTTGTTTGTTTTTCTCCATTAGAAGAGCAGTTTCTGCATGAATATGAGCCTTTTCTTCATCTGTTAACATGAGA
GTTGGAGAGGAAACTACTGGCTTGGCGGAACAATCAGGAATAACTTTTTCATTCTGAGGAGACTGTTGCTCATGAT
TTGTACTTTCAGATTTGATTATATGCTCTTCAGGCAATTTGACTGCCAGTTTTTTAGTATGATCAATCTGTGGAAC
ATTCTTTATGCTAGGCACTGGCTGAATGATGGGTGAAACATCAGATTTAGAAGCAGCAACTGATTCAACTGGAATT
GATATATTCAGTGGTCCTGTCCTCTCATTTTGATCATTATCACTTATTTGATCACTTATCAATTCTATGTCTTCAT
CCACTTCTATAGGTGGAGGTGGCATCTCGGCAGCAGGTTTAGAAGGAATTGATTCTTCCAATGAGGGATAAGTAAA
ATCCAATGAGATAGACAACTCTTCATTCTGGTGTTGTGGGGGTGGAGTGACCTTAGCATTTGTTGTATACTGGGAA
TAGCAAAGGAACCAGTTTTTATAGCCTCCCTCTAAAACCAAAGGCCCAAGGCCCATTGCACAGGACAGTTTTACTT
TCCCACTTGAAAAGTGCATCTTTCAGATGCCAGAGTGTTGTTCCAATCTGTAAATCTTTTGCAGAACTAAACCAGT
CAAGAAGTACCATATACTCCACATTCCCCCTCTTCTTCCATGTATCTATAGAATCATCTGGGAGGTGTGCTTCAAT
CCAGCTAGCAGTGACTCCTGGACTGATGGCTTTTCCAGGAACACTGAGAGAATGTAAAATACAGGAATCCTGATAA
TCCTGCATTCTTTGAGCATCCATTATAATCAAGCTGATGTTTTTATCCATCATCATTGTGTATAGTTCCTTTGCTG
TGATTGCTCCTTTCTCTTTGGTCTCACATTTTTCATTCTTTTCACCATTGCTCTTTTGGGTTTTGTCTTTGGAATC
CAATACAATCTCCAAAGAATCTTTAGCCAACGTGCTGCCATCCTCTCTTCCTGTTTCCTGCCTTTTTTGTTGTAGC
CACTGTGCTTCCCCCTGCCTGTCCTTTTCCTCAAGTTTTTTCCAGATTTCAACTTCTTCATATCTTAGTTTAAGGC
TTTCAGAGAGTCGTTCAGTTTCTCCAGTGGCTTTTTTGATGTTTGCAGGTCCAAGTATTGAATGGAAGTAATACTG
CTGTTGCTTGAAATCAGTTCTTTTTTGGATAGGATTATAAACAGTCATGTTTCATATACATGTGTGAGCTTTTTTT
CATATGTTTCTTGGCCACGTAAATGTCTTCCTCTGAGAAGTGTCTGTTTATATCCTTTGCCCACTTTTTGATGGGG
ATGGGTTTTTTTCTTGTAAATTTGTTTAAGTTCCTTGTAAACAAACATGTGAGCTCTCATCATTCTTGTTTAAACA
CCTAACAGTCATCCTAACCAGTGCAACAAGGACCAGGCATGGTGGCTCATGCCTGTAATCCCTGCATTTTGGGAGG
CTGAGGTGGGAGGATCACTTGAGATCAGGAGTTTGAGACCAGCCTGATAAGTGAGACCTCATCTCTACCAAATAAA
AATAATTTTAAAAGAAAATAGATCAATGGATAAGAAAGGAAGAAATGAAAGTCTTTCTTTGTCACCAGCTTCATTG
TATATGTAGAAAACACTAGGGAATTCTGAAAAAGTCTCTGGAATTAATCATTGAATTTGCAAAATAGTTCATAAAA
TATATGTAATAAGTCGCTTAGATGAACATGAAAAGATAGCAAACAATACTAGTCATCAAAGAAGTGCAAGTTAAAA
CCACAATGAGAAACCATCACACATCACCTAGAGCAGGTAAAGTTAAAAAGACATATGATAAGTCTTAACACTGGCA
AGAATATGGAAAAAATAGGAATGTCTGATATTGCTGGTAGGAATGCAAAAAATGTGGCAGCCAATTTGTAAAGTGG
TATGGCAGTTTCTTATACAGTTACCCATCTATTACCACATGGCCCAGCAATTCCACAAATACGTATTTATCCAAAA
GAAATAAAAATGTAAGTCCACACTTGTAAGCAGTTATTTATAGTGGCTTCATTAATAACAAGCCCTAACTGGAGGA
ATCCACATGTCTATCAACTGGAGACACAGAAACCAATGAATAAACTGGGATTCCAGCAATACTCGGCAGCTGCTCA
GCAACAAAAATGAATGAATGGCATCATCTCAAACATCGTTATGCTAAGGGAGAGACCAAACAAAAGACTACATAAC
ATATGATTGCATGTCCATGAAATTCTAGAAATGTCACTATTGCAGTGACAGAAAGCACAGCAGTGGTTGAATGAAG
GGAAGGGGGTGAGGGTGGGAGGCAAGGATTAAATAGAAAGGGGCATAAAGAAAGTTTTTAGGGAAAAGAAACTGTC
CTCTACAGCGCCACAACTCAGGAGTGACTGGGAGGGGGAAGTAAGGGGAGAAGAAGGTCTGAGGGATAAGGGGCAG
AGAGAAGGGCTGGGGAAGCAGGAGGAGAGGACGAGGAGCAGGGGAAAGGACTCTAAAGCAGTGGAGGGGCCTAGTA
GGAGGATCTTTGCATTTGGTGTTTCTCTACTGGGCAGTGTGGTAGTTACACTATAAATAATTACCAATATCCACCA
AAAAGTGCAGCTAAAACTGGTGAATTTTTTTACACGTAAACGCCCTGATAAGCAAAAAAAAAAAAAAAAAAAAAAA
AAAAAAAAAAAAAAAAGGGGGGGGGGGAGGAAAGAAGGCAAAAATAAAGACATTGTTAGATCATCAAACCCATAAA
ATTCATTTCCTAGGGGTCCTGTACTACATGTAATTTTAAAGGACGTTCTACAGGCTGAAGGGAAATGATACTAGAT
GGTGACCCAGATACACAGAAAGGAACAATTAACAACAGAAATGATGCACATACACATATCACATACACACCCATTT
TCTTAATTTCCTGAAGATACGTGACTGCTTGTCTAAAACAAAAAGTATTACACTGTATTGTTGAGTTTATAACGTA
TATTGATGTAACATATACAATAATAATAGCATAATGATAGTTTAAATGAAACTACACCATTTGAAGTGTCCTTTAT
TTTGCTGGATGCAGCTTAATATTACCTGAATTTCACTGTGAAAAGTCAAGGAATTGGGTTTCAATTCTTACAACAA
TAAAAAATTAATGTAAAGAAATATAGCTAAAAGCCACTAGGAGAATTAAAACCATAAGCTAAAAAATGTTTACTTG
AAACATAAGAAAGTAGCAAAGGAGGAACAGAAACAAAAAGATATGAGACAAATTGAAAACGTATAGCAAAATGGTA
GACCAAAATCCAACCATTATAAGTGAAGAAATGACACGACCTGAGTCACATTAGCAGGACTGCTGAGCACTATGGG
GAGAACAGACATGGGCAGGAAGTGAGGGACAGTGTTAGTGCCACAATTCAGGAGTGACAGGGTGGCGGGGACTAAA
GGGGAAAGAGGGTGTGAGGGATGAGAGGGGCAGACAGAAGGGCTGGAGAGGCAGGAGGTGAGGAAAAGGAGCAGAG
GAAAGAATTCTAAAGCAGTAGAAGAGCCTGGCAGGGGGTTCTTTGCATTCGGTATTTAATACATTTTGTGTGACTG
CCTTAAAACTAATGGGCTCCTTATGATTTTTTTTTAAAAAGGGGTTACAAAAATATCAAGTGTCCAAATAAAATAT
GCACACTGCTTAGATGTGCATAGTTCCGGAAAACGGGCAGTGCTGGAGCGCTGGTGAAGAGCATTGGGACTGCATG
GAGCCCTCACAACTTTGAGGTGATGACTACAGGCTCCCGGTTGCAATAGACAGTAACAAACCCTGCTTCTTTGTAT
TCAGGAGATGTTCTGGACTCACACAGGGAAACTCTGGCTAGAGAATGAGGATAACTTTAAATGCAACAACCCAGAG
TCACAGATCCATAGTCTGCGAAAGTAAAACAGGAGCTTTGAGAATTTAATTGTAATGCAGTTTTGACACAGGTCTT
TCACAGATTGGAATTCTAATCATTCAGGGATTACCAATATTGTGCTACCTACTGTATCAATAAACAAAAAGGAAAC
TGGTCTCTATGAGAATCTCTACCTGGTGCTTTCAGACAAAACTTCACCAGGTTTAAAGAGAAAACTCCTGACTCTA
CACGTCCATTCACAGGGCGAGCTCACTGTCTGGCATCAAGTTCCCCATGGTGAGTTTCCCTGTACAAGAGTCCAAG
GGGAGAGGTAAGTGTCCTTTATTTTGCTGGATGTAGTTTAATATTACCTGAGGTAAGGTAAGGCAAAGAGTGGGAG
GCAGGGAGTCCAGTTCAGGGACGGGGATTCCAGGAGAAGTGAAGGGGAAGGGGCTGGGCGCAGCCTGGGGGTCTCT
CCCTGGTTTCCACAGACAGATCCTTGGCCAGGACTCAGGCACACAGTGTGACAAAGATGCTTGGTGTAGGAGAAGA
GGAATCAGGACGAAGTCCCAGGTCCCGGGCGGGGCTCTCAGGGTCTCAGGCTCCAAGGGCCGTGTCTGCAATGGGG
AGGCGCCGCGTTGAGGATTCTCCACTCCCCTGAGTTTCACTTCTTCTCTCCCAACCTGCGTCGGGTCCTTCTTCCT
GAATACTCATGACGCGTCCCCAATTCCCACTCCCATTGGGTGTCGGATTCTAGAGAAGCCAATCAGCGTCTCCGCA
GTCCCGGTTCTGAAGTCCCCAGTCACCCACCCGGACTCAGATTCTCCCCAGACGCCGAGATGCGGGTCATGGCGCC
CCGAACCCTCATCCTGCTGCTCTCGGGAGCCCTGGCCCTGACCGAGACCTGGGCCGGTGAGTGCGGGGTTGGGAGG
GAATCGGCCTCTGCGGAGAGGAGCGAGGGGCCCGCCCGGCGAGGGCGCAGGACCCGGGGAGCAGCGCAGGGAGGAG
GGTCGGGCGGGTCTCAGCCCCTCCTCGCCCCCAGGCTCCCACTCCATGAGGTATTTCTACACCGCTGTGTCCCGGC
CCGGCCGCGGGGAGCCCCACTTCATCGCAGTGGGCTACGTGGACGACACGCAGTTCGTGCGGTTCGACAGCGACGC
CGCGAGTCCGAGAGGGGAGCCGCGGGCGCCGTGGGTGGAGCAGGAGGGGCCGGAGTATTGGGACCGGGAGACACAG
AAGTACAAGCGCCAGGCACAGACTGACCGAGTGAGCCTGCGGAACCTGCGCGGCTACTACAACCAGAGCGAGGCCA
GTGAGTGACCCCGGCCCGGGGCGCAGGTCACGACCCCTCCTCATCCCCCACGGACGGCCCGGGTCGCCCCAAGTCT
CCCGGTCTGAGATCCACCCCGAGGCTGCGGAACCCGCCCAGACCCTCGACCGGAGAGAGCCCCAGTCACCTTTACC
CGGTTTCATTTTCAGTTTAGGCCAAAATCCCCGCGGGTTGGTCGGGGCGGGGCGGGGCTCGGGGGACGGGGCTGAC
CGCGGGGGCGGGGCCAGGGTCTCACATCATCCAGAGGATGTATGGCTGCGACGTGGGGCCCGACGGGCGCCTCCTC
CGCGGGTATGACCAGTCCGCCTACGACGGCAAGGATTACATCGCCCTGAACGAGGATCTGCGCTCCTGGACCGCCG
CGGACACGGCGGCTCAGATCACCCAGCGCAAGTGGGAGGCGGCCCGTGAGGCGGAGCAGCTGAGAGCCTACCTGGA
GGGCCTGTGCGTGGAGTGGCTCCGCAGATACCTGAAGAATGGGAAGGAGACGCTGCAGCGCGCGGGTACCAGGGGC
AGTGGGGAGCCTTCCCCATCTCCTGTAGATCTCCCGGGATGGCCTCCCACGAGGAGGGGAGGAAAATGGGATCAGC
GCTAGAATATCGCCCTCCCTTGAATGGAGAATGGGATGAGTTTTCCTGAGTTTCCTCTGAGGGCCCCCTCTGCTCT
CTAGGACAATTAAGGGATGAAGTCCTTGAGGAAATGGAGGGGAAGACAGTCCCTAGAATACTGATCAGGGGTCCCC
TTTGACCACTTTGACCACTGCAGCAGCTGTGGTCAGGCTGCTGACCTTTCTCTCAGGCCTTGTTCTCTGCCTCACG
CTCAATGTGTTTGAAGGTTTGATTCCAGCTTTTCTGAGTCCTTCGGCCTCCACTCAGGTCAGGACCAGAAGTCGCT
GTTCCTCCCTCAGAGACTAGAACTTTCCAATGAATAGGAGATTATCCCAGGTGCCTGTGTCCAGGCTGGCGTCTGG
GTTCTGTGCCCCCTTCCCCACCCCAGGTGTCCTGTCCGTTCTCAGGATGGTCACATGGGCGCTGTTGGAGTGTCGC
AAGAGAGATACAAAGTGTCTGAATTTTCTGACTCTTCCCGTCAGAACACCCAAAGACACACGTGACCCACCATCCC
GTCTCTGACCATGAGGCCACCCTGAGGTGCTGGGCCCTGGGCTTCTACCCTGCGGAGATCACACTGACCTGGCAGT
GGGATGGGGAGGACCAAACTCAGGACACTGAGCTTGTGGAGACCAGGCCAGCAGGAGATGGAACCTTCCAGAAGTG
GGCAGCTGTGGTGGTGCCTTCTGGAGAAGAGCAGAGATACACGTGCCATGTGCAGCACGAGGGGCTGCCGGAGCCC
CTCACCCTGAGATGGGGTAAGGAGGGGGATGAGGGGTGATGTGTCTTCTCAGGGAAAGCAGAAGTCCTGGAGCCCT
TCAGCCAGGTCAGGGCTGAGGCTTGGGGGTCAGGGCCCCTCACCTTCCCCTCCTTTCCCAGAGCCGTCTTCCCAGC
CCACCATCCCCATCGTGGGCATCGTTGCTGGCCTGGCTGTCCTGGCTGTCCTAGCTGTCCTAGGAGCTGTGGTGGC
TGTTGTGATGTGTAGGAGGAAGAGCTCAGGTAGGGAAGGGGTGAGGAGTGGGGTCTGGGTTTTCTTGTTCCACTGG
GAGTTTCAAGCCCCAGGTAGAAGTGTGCCCCACCTCGTTACTGGAAGCACCATCCACACATGGGCCATCCCAGCCT
GGGACCCTGTGTGCCAGCACTTACTCTGTTGTGAAGCACATGACAATGAAGGACAGATGTATCACCTTGATGATTA
TGGTGTTGGGGTCCTTGATTCCAGCATTCATGAGTCAGGGGAAGGTCCCTGCTAAGGACAGACCTTAGGAGGGCAG
TTGCTCCAGAACCCACAGCTGCTTTCCCCGTGTTTCCTGATCCTGCCCTGGGTCTGCAGTCATAGTTCTGGAAACT
TCTCTTGGGTCCAAGACTAGGAGGTTCCCCTAAGATCGCATGGCCCTGACTCCTCCCTGTCCCCTCACAGGGCATT
TTCTTCCCACAGGTGGAAAAGGAGGGAGCTGCTCTCAGGCTGCGTGTAAGTGATGGCGGTGGGCGTGTGGAGGAGC
TCACCCACCCCATAATTCCTCTTGTCCCACATCTCCTGCGGGCTCTGACCAGGTCTTTTTTTTTGTTCTACCCCAG
CCAGCAACAGTGCCCAGGGCTCTGATGAGTCTCTCATCGCTTGTAAAGGTGAGATTCTGGGGAGCTGAAGTGGTCG
GGGGTGGGGCAGAGGGAAAAGGCCTAGGTAATGGGGATCCTTTGATTGGGACGTTTCGAATGTGTGGTGAGCTGTT
CAGAGTGTGATCACTTACCATGACTGACCTGAATTTGTTCATGACTATTGTGTTCTGTAGCCTGAGACAGCTGCCT
GTGTGGGACTGAGATGCAGGATTTCTTCACACCTCTCCTTTGTGACTTCAAGAGCCTCTGGCATCTCTTTCTGCAA
AGGCATCTGAATGTGTCTGCGTTCCTGTTAGCATAATGTGAGGAGGTGGACAGACAGCCCACCCCCGTGTCCACCG
TGACCCCTGTCCCCACACTGACCTGTGTTCCCTCCCCGATCATCTTTCCTGTTCCAGAGAAGTGGGCTGGATGTCT
CCATCTCTGTCTCAACTTCATGGTGCGCTGAGCTGCAACTTCTTACTTCCCTAATGAAGTTAGGAACCTGAATATA
AATTTGTTTTCTCAAATATTTGCTATGAAGGGTTGATGGATTAATTAAATAAGTCAATTCCTGGAAGTTGAGAGAG
CAAATAAAGACCTGAGAACTTTCCAGAATCCGCATGTTCTCTGTGCTGAGTCTGTTGCAGGTGGGGGTGGGGAAGG
CTGTGAGGAGCCGAGTGTGGACGGGGGCCTGTGCCTAGTTGCTGTTCAGTTCTTCATGGGCTTTATGTAGTCAGTC
CTTAGCTGGGTCACCTTCACTGCTCCATTGTCCTTGTCCCTTCAGTGGAAACTTGTCCAGCAGGAGCTGTGACCAC
AGAGGCTCACACATCGCCCAGGGCAGCCCCTGCACACGGGAGTCCCTGTGCTTTCTGAGACAAATTTTCAGACCCA
TTCAGCTCCTGCCCTCCTTATAGGGCTCCTCTTCTGCTTTGGTCTCCTGCCCTCTCTCCCTTCCCTGATTCCAGTG
ATCTTCGTGCTGACTCCAATCCCAACTCATGAATCTAAAGCAGAGCCTAATTTAGATTTGTATTTGTTTGTAAAAT
TGGGTCCATAGTCTAGAATTGTTCCTTCCTGAAGAGAGAAACCTGATCGTGTGCTGCAGCGTGCAGGGCGGTTGGT
GTGGGAGGAGGGATAGGGGAGGGAGGACACACAAGCAGCCCTGCTGAGAAAAGTACAGGCGGCCTCGGTGTCAGTG
TGAGGGGACCTTGTGCTGCAGCTGCCACAAAACAGCACTTGGCCTGAGGCTATGTTAATAAAGATACTGGCTTTAG
AATAGGAGGTGCTCTACACTGATCATTCAACTGACCTTTGTTGTCAGCCAGACACAGGACAGAAAAGTTCTGCATC
TGGGGAACACCATTGAAGTAAAATCAGAAAAATATCTGAGCATATGCTCCAGTGGTAAGAGGCAGACGATACATAC
ACTATAACCACAGTAAGAAAAGAAAGTGATGGAAGGTGGTAAGTGCCATGAGGCAGGTGATCCGGGTATGGGCAGT
GGGGACAGGGAAGGTGGCTGTTGGACAGGAGTTGTCAATGTGTGCCTTGTTGCAAAGATGACCTTTGAGGAAAGAT
TTGAGGGACATGAGGATGTCTGGGGAAGTTCTTTCTAGGCAAGGAAACACAACTTCGTATAGAAAGGTATATACGA
AGTTGT
(LANDING PAD)
SEQ ID NO: 2
GAAGTTCCTATACTCTAGAAAGAATAGGAACTTCGTCGACCCAGACATGATAAGATACATTGATGAGTTTGGACAA
ACCACAACTAGAATGCAGTGAAAAAAATGCTTTATTTGTGAAATTTGTGATGCTATTGCTTTATTTGTAACCATTA
TAAGCTGCAATAAACAAGTTAACAACAACAATTGCATTCATTTTATGTTTCAGGTTCAGGGGGAGGTGTGGGAGGT
TTTTTAAAGCAAGTAAAACCTCTACAAATGTGGTATGGCTGATTATGATCTTAGTCCCCATCCTCGAGCAGCCTCA
CCATGGCCCCAGTCTCAGAGTCCAGGTTTCTGATGTAGTTCATCACAATGTTCACATTGGTCCAGCCACCAGCCTG
CATGATTTCAGGGATGGACACACCAGCCCTGGCCATGTCCCTGGCAGCACCCACTCTGGCAGAGTGGCCAGACCAG
GCCAGGTATCTCTGCCCAGAGTCATCCTTGGCACCATAGATCAGGCGGTGGGTGGCCTCAAAGATCCCTTCCAGGG
CCCGGGTGGACAGTTGGGAGGTGGCAGAAGGGGCAGCCACACCATTCTTTCTGACCCGGCAGAACAGGTAGTTGTT
GGGGTCATCAGCCACACCAGACACAGAGATCCATCTCTCCACCAGCTTGGTAACCCCCAGGGACAGGGCCTTCTCC
ACACCAGCTGTGGACACCAGGGTCTTGGTCCTGCCAATGTGGATCAGCATTCTCCCACCATCGGTGCGGGAGATGT
CCTTCACTCTGATTCTGGCAATTTCGGCAATGCGCAGCAGGGTGTTGTAGGCAATGCCCAGGAAGGCCAGGTTCCT
GATGTCCTGGCATCTGTCAGAGTTCTCCATCAGGGATCTGACTTGGTCAAAGTCAGTGCGTTCAAAGGCCAGGGCC
TGCTTGGCTCTCTCCCCAGCATCCACATTCTCCTTTCTGATTCTCCTCATCACCAGGGACACAGCATTGGAGTCAG
AAGGGCGAGGCAGGCCAGATCTCCTGTGCAGCATGTTGAGCTGGCCCAGGTGCTGTTGGATGGTCTTCACAGCCAG
GCCTCTGGCTTGCAGGTACAGGAGGTAGTCCCTCACATCCTCAGGTTCAGCAGGGAACCATTTCCTGTTGTTCAGC
TTGCACCAGGCAGCCCAGGATCTGCACACAGACAGGAGCATCTTCCAGGTGTGTTCAGAGAAGGCCTGCCTGTCCC
TGAACATGTCCATCAGGTTCTTCCTGACTTCATCAGAGGTGGCATCCACAGGGAGGGCAGGCAGGTTTTGGTGCAC
AGTCAGCAGGTTGGAGACTTTCCTCTTCTTCTTGGGCACCATGGTGGCGAATTCTTTACGAGGGTAGGAAGTGGTA
CGGAAAGTTGGTATAAGACAAAAGTGTTGTGGAATTGAAGTTTACTCAAAAAATCAGCACTCTTTTATAGGCGCCC
TGGTTTACATAAGCAAAGCTTATACGTTCTCTATCACTGATAGGGAGTAAACTGGATATACGTTCTCTATCACTGA
TAGGGAGTAAACTGTAGATACGTTCTCTATCACTGATAGGGAGTAAACTGGTCATACGTTCTCTATCACTGATAGG
GAGTAAACTCCTTATACGTTCTCTATCACTGATAGGGAGTAAAGTCTGCATACGTTCTCTATCACTGATAGGGAGT
AAACTCTTCATACGTTCTCTATCACTGATAGGGAGTAAACTCGAGGGGATCCTGAAATTAACGCCGACGGGAGGGG
CTTAATCCGCAGCCTGGAGATCCAGCCCCCTCAACCCGGGAGGTGGTCCCTGCAGTTACGCCAATGATAACCCCCG
CCAGAAAAATCTTAGTAGCCTTCCCTTTTTGTTTTCCGTGCCCCAACTCGGCGGATTGACTCGGCCCCTTCCGGAA
ACACCCGAATCAACTTCTAGTCAAATTATTGTTCACGCCGCAATGACCCACCCCTGGCCCGCGTCTGTGGAACTGA
CCCCTGGTGTACAGGAGAGTTCGCTGCTGAAAGTGGTCCCAAAGGGGTACTAGTTTTTAAGCTCCCAACTCCCCCT
CCCCCAGCGTCTGGAGGATTCCACACCCTCGCACCGCAGGGGCGAGGAAGTGGGCGGAGTCCGGTTTTGGCGCCAG
CCGCTGAGGCTGCCAAGCAGAAAAGCCACCGCTGAGGAGACTCCGGTCACTGTCCTCGCCCCGCCTCCCCCTTCCC
TCCCCTTGGGGACCACCGGGCGCCACGCCGCGAACGGTAAGTGCCGCGGTCGTCGGCGCCTCCGCCCTCCCCCTAG
GGCCCCAATTCCCAGCGGGCGCGGCGCGCGGCCCCTCCCCCCGCCGGGCGCGCGCCCGCTGCCCCGCCCTTCGTGG
CCGCCCGGCGTGGGCGGTGCCACCCCTCCCCCCGGCGGCCCCGCGCGCAGCTCCCGGCTCCCTCCCCCTTCGGATG
TGGCTTGAGCTGTAGGCGCGGAGGGCCGGAGACGCTGCAGACCCGCGACCCGGAGCAGCTCGGAGGCGGTGAAGTC
GGTGGCTTTCCTTCTCTCTAGCTCTCGCTCGCTGGTGGTGCTTCAGATGCCACACGCGTCCCGGGGGCCCGGTTCT
CCGCTCCCCTCGGCTCCGGTGCCCGTCAGTGGGCAGAGCGCACATCGCCCACAGTCCCCGAGAAGTTGGGGGGAGG
GGTCGGCAATTGAACCGGTGCCTAGAGAAGGTGGCGCGGGGTAAACTGGGAAAGTGATGTCGTGTACTGGCTCCGC
CTTTTTCCCGAGGGTGGGGGAGAACCGTATATAAGTGCAGTAGTCGCCGTGAACGTTCTTTTTCGCAACGGGTTTG
CCGCCAGAACACAGGTAAGTGCCGTGTGTGGTTCCCGCGGGCCTGGCCTCTTTACGGGTTATGGCCCTTGCGTGCC
TTGAATTACTTCCACCTGGCTGCAGTACGTGATTCTTGATCCCGAGCTTCGGGTTGGAAGTGGGTGGGAGAGTTCG
AGGCCTTGCGCTTAAGGAGCCCCTTCGCCTCGTGCATGAGTTGAGGCCTGGCCTGGGCGCTGGGGCCGCCGCGTGC
GAATCTGGTGGCACCTTCGCGCCTGTCTCGCTGCTTTCGATAAGTCTCTAGCCATTTAAAATTTTTGATGACCTGC
TGCGACGCTTTTTTTCTGGCAAGATAGTCTTGTAAATGCGGGCCAAGATCTGCACACTGGTATTTCGGTTTTTGGG
GCCGCGGGCGGCGACGGGGCCCGTGCGTCCCAGCGCACATGTTCGGCGAGGCGGGGCCTGCGAGCGCGGCCACCGA
GAATCGGACGGGGGTAGTCTCAAGCTGGCCGGCCTGCTCTGGTGCCTGGCCTCGCGCCGCCGTGTATCGCCCCGCC
CTGGGCGGCAAGGCTGGCCCGGTCGGCACCAGTTGCGTGAGCGGAAAGATGGCCGCTTCCCGGCCCTGCTGCAGGG
AGCTCAAAATGGAGGACGCGGCGCTCGGGAGAGCGGGCGGGTGAGTCACCCACACAAAGGAAAAGGGCCTTTCCGT
CCTCAGCCGTCGCTTCATGTGACTCCACGGAGTACCGGGCGCCGTCCAGGCACCTCGATTAGTTCTCGAGCTTTTG
GAGTACGTCGTCTTTAGGTTGGGGGGAGGGGTTTTATGCGATGGAGTTTCCCCACACTGAGTGGGTGGAGACTGAA
GTTAGGCCAGCTTGGCACTTGATGTAATTCTCCTTGGAATTTGCCCTTTTTGAGTTTGGATCTTGGTTCATTCTCA
AGCCTCAGACAGTGGTTCAAAGTTTTTTTCTTCCATTTCAGGTGTCGTGAGCCACCATGGGATCTAGACTGGACAA
GAGCAAAGTCATAAACTCTGCTCTGGAATTACTCAATGGAGTCGGTATCGAAGGCCTGACGACAAGGAAACTCGCT
CAAAAGCTGGGAGTTGAGCAGCCTACCCTGTACTGGCACGTGAAGAACAAGCGGGCCCTGCTCGATGCCCTGCCAA
TCGAGATGCTGGACAGGCATCATACCCACTCCTGCCCCCTGGAAGGCGAGTCATGGCAAGACTTTCTGCGGAACAA
CGCCAAGTCATACCGCTGTGCTCTCCTCTCACATCGCGACGGGGCTAAAGTGCATCTCGGCACCCGCCCAACAGAG
AAACAGTACGAAACCCTGGAAAATCAGCTCGCGTTCCTGTGTCAGCAAGGCTTCTCCCTGGAGAACGCACTGTACG
CTCTGTCCGCCGTGGGCCACTTTACACTGGGCTGCGTATTGGAGGAACAGGAGCATCAAGTAGCAAAAGAGGAAAG
AGAGACACCTACCACCGATTCTATGCCCCCACTTCTGAAACAAGCAATTGAGCTGTTCGACCGGCAGGGAGCCGAA
CCTGCCTTCCTTTTCGGCCTGGAACTAATCATATGTGGCCTGGAGAAACAGCTAAAGTGCGAAAGCGGCGGGCCGA
CCGACGCCCTTGACGATTTTGACTTAGACATGCTCCCAGCCGATGCCCTTGACGACTTTGACCTTGATATGCTGCC
TGCTGACGCTCTTGACGATTTTGACCTTGACATGCTCCCCGGGGGCTCAGGCGCCACCAACTTTAGCCTGCTGAAG
CAGGCAGGCGACGTCGAGGAGAACCCCGGCCCTATAACTTCGTATAGCATACATTATACGAAGTTATCCAAAACAT
TTAACATTTCTCAACAAGATCTAGAATTAGTAGAAGTAGCGACAGAGAAGATTACAATGCTTTATGAGGATAATAA
ACATCATGTGGGAGCGGCAATTCGTACGAAAACAGGAGAAATCATTTCGGCAGTACATATTGAAGCGTATATAGGA
CGAGTAACTGTTTGTGCAGAAGCCATTGCGATTGGTAGTGCAGTTTCGAATGGACAAAAGGATTTTGACACGATTG
TAGCTGTTAGACACCCTTATTCTGACGAAGTAGATAGAAGTATTCGAGTGGTAAGTCCTTGTGGTATGTGTAGGGA
GTTGATTTCAGACTATGCACCAGATTGTTTTGTGTTAATAGAAATGAATGGCAAGTTAGTCAAAACTACGATTGAA
GAACTCATTCCACTCAAATATACCCGAAATGGCAGTGGAGAGGGCAGAGGAAGTCTGCTAACATGCGGTGACGTCG
AGGAGAATCCTGGCCCAGTGAGCAAGGGCGAGGCAGTGATCAAGGAGTTCATGCGGTTCAAGGTGCACATGGAGGG
CTCCATGAACGGCCACGAGTTCGAGATCGAGGGCGAGGGCGAGGGCCGCCCCTACGAGGGCACCCAGACCGCCAAG
CTGAAGGTGACCAAGGGTGGCCCCCTGCCCTTCTCCTGGGACATCCTGTCCCCTCAGTTCATGTACGGCTCCAGGG
CCTTCACCAAGCACCCCGCCGACATCCCCGACTACTATAAGCAGTCCTTCCCCGAGGGCTTCAAGTGGGAGCGCGT
GATGAACTTCGAGGACGGCGGCGCCGTGACCGTGACCCAGGACACCTCCCTGGAGGACGGCACCCTGATCTACAAG
GTGAAGCTCCGCGGCACCAACTTCCCTCCTGACGGCCCCGTAATGCAGAAGAAAACAATGGGCTGGGAAGCGTCCA
CCGAGCGGTTGTACCCCGAGGACGGCGTGCTGAAGGGCGACATTAAGATGGCCCTGCGCCTGAAGGACGGCGGCCG
CTACCTGGCGGACTTCAAGACCACCTACAAGGCCAAGAAGCCCGTGCAGATGCCCGGCGCCTACAACGTTGACCGC
AAGTTGGACATCACCTCCCACAACGAGGACTACACCGTGGTGGAACAGTACGAACGCTCCGAGGGCCGCCACTCCA
CCGGCGGCATGGACGAGCTGTACAAGTGACCTCGACTGTGCCTTCTAGTTGCCAGCCATCTGTTGTTTGCCCCTCC
CCCGTGCCTTCCTTGACCCTGGAAGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAAGGGGGAGGATTGGGAAGACAATAG
CAGGCATGCTGGGGATGCGGTGGGCTCTATGGCATGCACAACTTCGTATAGAAAGGTATATACGAAGTTGT
SSODN
(HLA-A SsODN)
SEQ ID NO: 3
T*A*TAATTTTGCTTTTAACATATATTCAAATCCTTTGATACTGTTCCTTTCATATGGGCTTTAGAATAGGGAGGT
GCTCTACAGTGATCATTCA*T*T
*represents a phosphorothioate bond.
(HLA-B/C ssODN)
SEQ ID NO: 4
G*A*TGCACATACACATGTCACATTCACACTCATTTTCTTAATTTCCTGAAGGGCAAGGAAACTCCAGTCCAAATG
CACAATGGCAGGAAGGTGTCTGTG*T*T
*represents a phosphorothioate bond.
(LANDING PAD)
SEQ ID NO: 5
          1 TTATTATCAT GACATTAACC TATAAAAATA GGCGTATCAC GAGGCCCTTT GTCACAGCTT
        61 GTCTGTAAGC GGATGCCGGG AGCAGACAAG CCCGTCAGGG CGCGTCAGCG GGTGTTGGCG
      121 GGTGTCGGGG CTGGCTTAAC TATGCGGCAT CAGAGCAGAT TGTACTGAGA GTGCACCATA
      181 CCACAGCTTT TCAATTCAAT TCATCATTTT TTTTTTATTC TTTTTTTTGA TTTCGGTTTC
      241 TTTGAAATTT TTTTGATTCG GTAATCTCCG AACAGAAGGA AGAACGAAGG AAGGAGCACA
      301 GACTTAGATT GGTATATATA CGCATATGTA GTGTTGAAGA AACATGAAAT TGCCCAGTAT
      361 TCTTAACCCA ACTGCACAGA ACAAAAACCT GCAGGAAACG AAGATAAATC ATGTCGAAAG
      421 CTACATATAA GGAACGTGCT GCTACTCATC CTAGTCCTGT TGCTGCCAAG CTATTTAATA
      481 TCATGCACGA AAAGCAAACA AACTTGTGTG CTTCATTGGA TGTTCGTACC ACCAAGGAAT
      541 TACTGGAGTT AGTTGAAGCA TTAGGTCCCA AAATTTGTTT ACTAAAAACA CATGTGGATA
      601 TCTTGACTGA TTTTTCCATG GAGGGCACAG TTAAGCCGCT AAAGGCATTA TCCGCCAAGT
      661 ACAATTTTTT ACTCTTCGAA GACAGAAAAT TTGCTGACAT TGGTAATACA GTCAAATTGC
      721 AGTACTCTGC GGGTGTATAC AGAATAGCAG AATGGGCAGA CATTACGAAT GCACACGGTG
      781 TGGTGGGCCC AGGTATTGTT AGCGGTTTGA AGCAGGCGGC AGAAGAAGTA ACAAAGGAAC
      841 CTAGAGGCCT TTTGATGTTA GCAGAATTGT CATGCAAGGG CTCCCTATCT ACTGGAGAAT
      901 ATACTAAGGG TACTGTTGAC ATTGCGAAGA GCGACAAAGA TTTTGTTATC GGCTTTATTG
      961 CTCAAAGAGA CATGGGTGGA AGAGATGAAG GTTACGATTG GTTGATTATG ACACCCGGTG
    1021 TGGGTTTAGA TGACAAGGGA GATGCATTGG GTCAACAGTA TAGAACCGTG GATGATGTGG
    1081 TGTCTACAGG ATCTGACATT ATTATTGTTG GAAGAGGACT ATTTGCAAAG GGAAGGGATG
    1141 CTAAGGTAGA GGGTGAACGT TACAGAAAAG CAGGCTGGGA AGCATATTTG AGAAGATGCG
    1201 GCCAGCAAAA CTAAAAAACT GTATTATAAG TAAATGCATG TATACTAAAC TCACAAATTA
    1261 GAGCTTCAAT TTAATTATAT CAGTTATTAC CCTATGCGGT GTGAAATACC GCACAGATGC
    1321 GTAAGGAGAA AATACCGCAT CAGGAAATTG TAAACGTTAA TATTTTGTTA AAATTCGCGT
    1381 TAAATTTTTG TTAAATCAGC TCATTTTTTA ACCAATAGGC CGAAATCGGC AAAATCCCTT
    1441 ATAAATCAAA AGAATAGACC GAGATAGGGT TGAGTGTTGT TCCAGTTTGG AACAAGAGTC
    1501 CACTATTAAA GAACGTGGAC TCCAACGTCA AAGGGCGAAA AACCGTCTAT CAGGGCGATG
    1561 GCCCACTACG TGAACCATCA CCCTAATCAA GTTTTTTGGG GTCGAGGTGC CGTAAAGCAC
    1621 TAAATCGGAA CCCTAAAGGG AGCCCCCGAT TTAGAGCTTG ACGGGGAAAG CCGGCGAACG
    1681 TGGCGAGAAA GGAAGGGAAG AAAGCGAAAG GAGCGGGCGC TAGGGCGCTG GCAAGTGTAG
    1741 CGGTCACGCT GCGCGTAACC ACCACACCCG CCGCGCTTAA TGCGCCGCTA CAGGGCGCGT
    1801 CGCGCCATTC GCCATTCAGG CTGCGCAACT GTTGGGAAGG GCGATCGGTG CGGGCCTCTT
    1861 CGCTATTACG CCAGCTGGCG AAAGGGGGAT GTGCTGCAAG GCGATTAAGT TGGGTAACGC
    1921 CAGGGTTTTC CCAGTCACGA CGTTGTAAAA CGACGGCCAG TGAGCGCGCG TAATACGACT
    1981 CACTATAGGG CGAATTGGGT ACCGCGAGAC CTTCCTGGTC TCAGAAGTTC CTATACTCTA
    2041 GAAAGAATAG GAACTTCGTC GACCCAGACA TGATAAGATA CATTGATGAG TTTGGACAAA
    2101 CCACAACTAG AATGCAGTGA AAAAAATGCT TTATTTGTGA AATTTGTGAT GCTATTGCTT
    2161 TATTTGTAAC CATTATAAGC TGCAATAAAC AAGTTAACAA CAACAATTGC ATTCATTTTA
    2221 TGTTTCAGGT TCAGGGGGAG GTGTGGGAGG TTTTTTAAAG CAAGTAAAAC CTCTACAAAT
    2281 GTGGTATGGC TGATTATGAT CTTAGTCCCC ATCCTCGAGC AGCCTCACCA TGGCCCCAGT
    2341 CTCAGAGTCC AGGTTTCTGA TGTAGTTCAT CACAATGTTC ACATTGGTCC AGCCACCAGC
    2401 CTGCATGATT TCAGGGATGG ACACACCAGC CCTGGCCATG TCCCTGGCAG CACCCACTCT
    2461 GGCAGAGTGG CCAGACCAGG CCAGGTATCT CTGCCCAGAG TCATCCTTGG CACCATAGAT
    2521 CAGGCGGTGG GTGGCCTCAA AGATCCCTTC CAGGGCCCGG GTGGACAGTT GGGAGGTGGC
    2581 AGAAGGGGCA GCCACACCAT TCTTTCTGAC CCGGCAGAAC AGGTAGTTGT TGGGGTCATC
    2641 AGCCACACCA GACACAGAGA TCCATCTCTC CACCAGCTTG GTAACCCCCA GGGACAGGGC
    2701 CTTCTCCACA CCAGCTGTGG ACACCAGGGT CTTGGTCCTG CCAATGTGGA TCAGCATTCT
    2761 CCCACCATCG GTGCGGGAGA TGTCCTTCAC TCTGATTCTG GCAATTTCGG CAATGCGCAG
    2821 CAGGGTGTTG TAGGCAATGC CCAGGAAGGC CAGGTTCCTG ATGTCCTGGC ATCTGTCAGA
    2881 GTTCTCCATC AGGGATCTGA CTTGGTCAAA GTCAGTGCGT TCAAAGGCCA GGGCCTGCTT
    2941 GGCTCTCTCC CCAGCATCCA CATTCTCCTT TCTGATTCTC CTCATCACCA GGGACACAGC
    3001 ATTGGAGTCA GAAGGGCGAG GCAGGCCAGA TCTCCTGTGC AGCATGTTGA GCTGGCCCAG
    3061 GTGCTGTTGG ATGGTCTTCA CAGCCAGGCC TCTGGCTTGC AGGTACAGGA GGTAGTCCCT
    3121 CACATCCTCA GGTTCAGCAG GGAACCATTT CCTGTTGTTC AGCTTGCACC AGGCAGCCCA
    3181 GGATCTGCAC ACAGACAGGA GCATCTTCCA GGTGTGTTCA GAGAAGGCCT GCCTGTCCCT
    3241 GAACATGTCC ATCAGGTTCT TCCTGACTTC ATCAGAGGTG GCATCCACAG GGAGGGCAGG
    3301 CAGGTTTTGG TGCACAGTCA GCAGGTTGGA GACTTTCCTC TTCTTCTTGG GCACCATGGT
    3361 GGCGAATTCT TTACGAGGGT AGGAAGTGGT ACGGAAAGTT GGTATAAGAC AAAAGTGTTG
    3421 TGGAATTGAA GTTTACTCAA AAAATCAGCA CTCTTTTATA GGCGCCCTGG TTTACATAAG
    3481 CAAAGCTTAT ACGTTCTCTA TCACTGATAG GGAGTAAACT GGATATACGT TCTCTATCAC
    3541 TGATAGGGAG TAAACTGTAG ATACGTTCTC TATCACTGAT AGGGAGTAAA CTGGTCATAC
    3601 GTTCTCTATC ACTGATAGGG AGTAAACTCC TTATACGTTC TCTATCACTG ATAGGGAGTA
    3661 AAGTCTGCAT ACGTTCTCTA TCACTGATAG GGAGTAAACT CTTCATACGT TCTCTATCAC
    3721 TGATAGGGAG TAAACTCGAG GGGATCCTGA AATTAACGCC GACGGGAGGG GOTTAATCCG
    3781 CAGCCTGGAG ATCCAGCCCC CTCAACCCGG GAGGTGGTCC CTGCAGTTAC GCCAATGATA
    3841 ACCCCCGCCA GAAAAATCTT AGTAGCCTTC CCTTTTTGTT TTCCGTGCCC CAACTCGGCG
    3901 GATTGACTCG GCCCCTTCCG GAAACACCCG AATCAACTTC TAGTCAAATT ATTGTTCACG
    3961 CCGCAATGAC CCACCCCTGG CCCGCGTCTG TGGAACTGAC CCCTGGTGTA CAGGAGAGTT
    4021 CGCTGCTGAA AGTGGTCCCA AAGGGGTACT AGTTTTTAAG CTCCCAACTC CCCCTCCCCC
    4081 AGCGTCTGGA GGATTCCACA CCCTCGCACC GCAGGGGCGA GGAAGTGGGC GGAGTCCGGT
    4141 TTTGGCGCCA GCCGCTGAGG CTGCCAAGCA GAAAAGCCAC CGCTGAGGAG ACTCCGGTCA
    4201 CTGTCCTCGC CCCGCCTCCC CCTTCCCTCC CCTTGGGGAC CACCGGGCGC CACGCCGCGA
    4261 ACGGTAAGTG CCGCGGTCGT CGGCGCCTCC GCCCTCCCCC TAGGGCCCCA ATTCCCAGCG
    4321 GGCGCGGCGC GCGGCCCCTC CCCCCGCCGG GCGCGCGCCC GCTGCCCCGC CCTTCGTGGC
    4381 CGCCCGGCGT GGGCGGTGCC ACCCCTCCCC CCGGCGGCCC CGCGCGCAGC TCCCGGCTCC
    4441 CTCCCCCTTC GGATGTGGCT TGAGCTGTAG GCGCGGAGGG CCGGAGACGC TGCAGACCCG
    4501 CGACCCGGAG CAGCTCGGAG GCGGTGAAGT CGGTGGCTTT CCTTCTCTCT AGCTCTCGCT
    4561 CGCTGGTGGT GCTTCAGATG CCACACGCGT CCCGGGGGCC CGGTTCTCCG CTCCCCTCGG
    4621 CTCCGGTGCC CGTCAGTGGG CAGAGCGCAC ATCGCCCACA GTCCCCGAGA AGTTGGGGGG
    4681 AGGGGTCGGC AATTGAACCG GTGCCTAGAG AAGGTGGCGC GGGGTAAACT GGGAAAGTGA
    4741 TGTCGTGTAC TGGCTCCGCC TTTTTCCCGA GGGTGGGGGA GAACCGTATA TAAGTGCAGT
    4801 AGTCGCCGTG AACGTTCTTT TTCGCAACGG GTTTGCCGCC AGAACACAGG TAAGTGCCGT
    4861 GTGTGGTTCC CGCGGGCCTG GCCTCTTTAC GGGTTATGGC CCTTGCGTGC CTTGAATTAC
    4921 TTCCACCTGG CTGCAGTACG TGATTCTTGA TCCCGAGCTT CGGGTTGGAA GTGGGTGGGA
    4981 GAGTTCGAGG CCTTGCGCTT AAGGAGCCCC TTCGCCTCGT GCATGAGTTG AGGCCTGGCC
    5041 TGGGCGCTGG GGCCGCCGCG TGCGAATCTG GTGGCACCTT CGCGCCTGTC TCGCTGCTTT
    5101 CGATAAGTCT CTAGCCATTT AAAATTTTTG ATGACCTGCT GCGACGCTTT TTTTCTGGCA
    5161 AGATAGTCTT GTAAATGCGG GCCAAGATCT GCACACTGGT ATTTCGGTTT TTGGGGCCGC
    5221 GGGCGGCGAC GGGGCCCGTG CGTCCCAGCG CACATGTTCG GCGAGGCGGG GCCTGCGAGC
    5281 GCGGCCACCG AGAATCGGAC GGGGGTAGTC TCAAGCTGGC CGGCCTGCTC TGGTGCCTGG
    5341 CCTCGCGCCG CCGTGTATCG CCCCGCCCTG GGCGGCAAGG CTGGCCCGGT CGGCACCAGT
    5401 TGCGTGAGCG GAAAGATGGC CGCTTCCCGG CCCTGCTGCA GGGAGCTCAA AATGGAGGAC
    5461 GCGGCGCTCG GGAGAGCGGG CGGGTGAGTC ACCCACACAA AGGAAAAGGG CCTTTCCGTC
    5521 CTCAGCCGTC GCTTCATGTG ACTCCACGGA GTACCGGGCG CCGTCCAGGC ACCTCGATTA
    5581 GTTCTCGAGC TTTTGGAGTA CGTCGTCTTT AGGTTGGGGG GAGGGGTTTT ATGCGATGGA
    5641 GTTTCCCCAC ACTGAGTGGG TGGAGACTGA AGTTAGGCCA GCTTGGCACT TGATGTAATT
    5701 CTCCTTGGAA TTTGCCCTTT TTGAGTTTGG ATCTTGGTTC ATTCTCAAGC CTCAGACAGT
    5761 GGTTCAAAGT TTTTTTCTTC CATTTCAGGT GTCGTGAGCC ACCATGGGAT CTAGACTGGA
    5821 CAAGAGCAAA GTCATAAACT CTGCTCTGGA ATTACTCAAT GGAGTCGGTA TCGAAGGCCT
    5881 GACGACAAGG AAACTCGCTC AAAAGCTGGG AGTTGAGCAG CCTACCCTGT ACTGGCACGT
    5941 GAAGAACAAG CGGGCCCTGC TCGATGCCCT GCCAATCGAG ATGCTGGACA GGCATCATAC
    6001 CCACTCCTGC CCCCTGGAAG GCGAGTCATG GCAAGACTTT CTGCGGAACA ACGCCAAGTC
    6061 ATACCGCTGT GCTCTCCTCT CACATCGCGA CGGGGCTAAA GTGCATCTCG GCACCCGCCC
    6121 AACAGAGAAA CAGTACGAAA CCCTGGAAAA TCAGCTCGCG TTCCTGTGTC AGCAAGGCTT
    6181 CTCCCTGGAG AACGCACTGT ACGCTCTGTC CGCCGTGGGC CACTTTACAC TGGGCTGCGT
    6241 ATTGGAGGAA CAGGAGCATC AAGTAGCAAA AGAGGAAAGA GAGACACCTA CCACCGATTC
    6301 TATGCCCCCA CTTCTGAAAC AAGCAATTGA GCTGTTCGAC CGGCAGGGAG CCGAACCTGC
    6361 CTTCCTTTTC GGCCTGGAAC TAATCATATG TGGCCTGGAG AAACAGCTAA AGTGCGAAAG
    6421 CGGCGGGCCG ACCGACGCCC TTGACGATTT TGACTTAGAC ATGCTCCCAG CCGATGCCCT
    6481 TGACGACTTT GACCTTGATA TGCTGCCTGC TGACGCTCTT GACGATTTTG ACCTTGACAT
    6541 GCTCCCCGGG GGCTCAGGCG CCACCAACTT TAGCCTGCTG AAGCAGGCAG GCGACGTCGA
    6601 GGAGAACCCC GGCCCTATAA CTTCGTATAG CATACATTAT ACGAAGTTAT CCAAAACATT
    6661 TAACATTTCT CAACAAGATC TAGAATTAGT AGAAGTAGCG ACAGAGAAGA TTACAATGCT
    6721 TTATGAGGAT AATAAACATC ATGTGGGAGC GGCAATTCGT ACGAAAACAG GAGAAATCAT
    6781 TTCGGCAGTA CATATTGAAG CGTATATAGG ACGAGTAACT GTTTGTGCAG AAGCCATTGC
    6841 GATTGGTAGT GCAGTTTCGA ATGGACAAAA GGATTTTGAC ACGATTGTAG CTGTTAGACA
    6901 CCCTTATTCT GACGAAGTAG ATAGAAGTAT TCGAGTGGTA AGTCCTTGTG GTATGTGTAG
    6961 GGAGTTGATT TCAGACTATG CACCAGATTG TTTTGTGTTA ATAGAAATGA ATGGCAAGTT
    7021 AGTCAAAACT ACGATTGAAG AACTCATTCC ACTCAAATAT ACCCGAAATG GCAGTGGAGA
    7081 GGGCAGAGGA AGTCTGCTAA CATGCGGTGA CGTCGAGGAG AATCCTGGCC CAGTGAGCAA
    7141 GGGCGAGGCA GTGATCAAGG AGTTCATGCG GTTCAAGGTG CACATGGAGG GCTCCATGAA
    7201 CGGCCACGAG TTCGAGATCG AGGGCGAGGG CGAGGGCCGC CCCTACGAGG GCACCCAGAC
    7261 CGCCAAGCTG AAGGTGACCA AGGGTGGCCC CCTGCCCTTC TCCTGGGACA TCCTGTCCCC
    7321 TCAGTTCATG TACGGCTCCA GGGCCTTCAC CAAGCACCCC GCCGACATCC CCGACTACTA
    7381 TAAGCAGTCC TTCCCCGAGG GCTTCAAGTG GGAGCGCGTG ATGAACTTCG AGGACGGCGG
    7441 CGCCGTGACC GTGACCCAGG ACACCTCCCT GGAGGACGGC ACCCTGATCT ACAAGGTGAA
    7501 GCTCCGCGGC ACCAACTTCC CTCCTGACGG CCCCGTAATG CAGAAGAAAA CAATGGGCTG
    7561 GGAAGCGTCC ACCGAGCGGT TGTACCCCGA GGACGGCGTG CTGAAGGGCG ACATTAAGAT
    7621 GGCCCTGCGC CTGAAGGACG GCGGCCGCTA CCTGGCGGAC TTCAAGACCA CCTACAAGGC
    7681 CAAGAAGCCC GTGCAGATGC CCGGCGCCTA CAACGTTGAC CGCAAGTTGG ACATCACCTC
    7741 CCACAACGAG GACTACACCG TGGTGGAACA GTACGAACGC TCCGAGGGCC GCCACTCCAC
    7801 CGGCGGCATG GACGAGCTGT ACAAGTGACC TCGACTGTGC CTTCTAGTTG CCAGCCATCT
    7861 GTTGTTTGCC CCTCCCCCGT GCCTTCCTTG ACCCTGGAAG GTGCCACTCC CACTGTCCTT
    7921 TCCTAATAAA ATGAGGAAAT TGCATCGCAT TGTCTGAGTA GGTGTCATTC TATTCTGGGG
    7981 GGTGGGGTGG GGCAGGACAG CAAGGGGGAG GATTGGGAAG ACAATAGCAG GCATGCTGGG
    8041 GATGCGGTGG GCTCTATGGC ATGCACAACT TCGTATAGAA AGGTATATAC GAAGTTGTAG
    8101 AGACCAACCT GGTCTCAGCT AGCTCCCTTT AGTGAGGGTT AATTGCGCGC TTGGCGTAAT
    8161 CATGGTCATA GCTGTTTCCT GTGTGAAATT GTTATCCGCT CACAATTCCA CACAACATAG
    8221 GAGCCGGAAG CATAAAGTGT AAAGCCTGGG GTGCCTAATG AGTGAGGTAA CTCACATTAA
    8281 TTGCGTTGCG CTCACTGCCC GCTTTCCAGT CGGGAAACCT GTCGTGCCAG CTGCATTAAT
    8341 GAATCGGCCA ACGCGCGGGG AGAGGCGGTT TGCGTATTGG GCGCTCTTCC GCTTCCTCGC
    8401 TCACTGACTC GCTGCGCTCG GTCGTTCGGC TGCGGCGAGC GGTATCAGCT CACTCAAAGG
    8461 CGGTAATACG GTTATCCACA GAATCAGGGG ATAACGCAGG AAAGAACATG TGAGCAAAAG
    8521 GCCAGCAAAA GGCCAGGAAC CGTAAAAAGG CCGCGTTGCT GGCGTTTTTC CATAGGCTCC
    8581 GCCCCCCTGA CGAGCATCAC AAAAATCGAC GCTCAAGTCA GAGGTGGCGA AACCCGACAG
    8641 GACTATAAAG ATACCAGGCG TTTCCCCCTG GAAGCTCCCT CGTGCGCTCT CCTGTTCCGA
    8701 CCCTGCCGCT TACCGGATAC CTGTCCGCCT TTCTCCCTTC GGGAAGCGTG GCGCTTTCTC
    8761 ATAGCTCACG CTGTAGGTAT CTCAGTTCGG TGTAGGTCGT TCGCTCCAAG CTGGGCTGTG
    8821 TGCACGAACC CCCCGTTCAG CCCGACCGCT GCGCCTTATC CGGTAACTAT CGTCTTGAGT
    8881 CCAACCCGGT AAGACACGAC TTATCGCCAC TGGCAGCAGC CACTGGTAAC AGGATTAGCA
    8941 GAGCGAGGTA TGTAGGCGGT GCTACAGAGT TCTTGAAGTG GTGGCCTAAC TACGGCTACA
    9001 CTAGAAGGAC AGTATTTGGT ATCTGCGCTC TGCTGAAGCC AGTTACCTTC GGAAAAAGAG
    9061 TTGGTAGCTC TTGATCCGGC AAACAAACCA CCGCTGGTAG CGGTGGTTTT TTTGTTTGCA
    9121 AGCAGCAGAT TACGCGCAGA AAAAAAGGAT CTCAAGAAGA TCCTTTGATC TTTTCTACGG
    9181 GGTCTGACGC TCAGTGGAAC GAAAACTCAC GTTAAGGGAT TTTGGTCATG AGATTATCAA
    9241 AAAGGATOTT CACCTAGATC CTTTTAAATT AAAAATGAAG TTTTAAATCA ATCTAAAGTA
    9301 TATATGAGTA AACTTGGTCT GACAGTTACC AATGOTTAAT CAGTGAGGCA CCTATCTCAG
    9361 CGATCTGTCT ATTTCGTTCA TCCATAGTTG CCTGACTCCC CGTCGTGTAG ATAACTACGA
    9421 TACGGGAGGG CTTACCATCT GGCCCCAGTG CTGCAATGAT ACCGCGAGAT CCACGCTCAC
    9481 CGGCTCCAGA TTTATCAGCA ATAAACCAGC CAGCCGGAAG GGCCGAGCGC AGAAGTGGTC
    9541 CTGCAACTTT ATCCGCCTCC ATCCAGTCTA TTAATTGTTG CCGGGAAGCT AGAGTAAGTA
    9601 GTTCGCCAGT TAATAGTTTG CGCAACGTTG TTGCCATTGC TACAGGCATC GTGGTGTCAC
    9661 GCTCGTCGTT TGGTATGGCT TCATTCAGCT CCGGTTCCCA ACGATCAAGG CGAGTTACAT
    9721 GATCCCCCAT GTTGTGCAAA AAAGCGGTTA GCTCCTTCGG TCCTCCGATC GTTGTCAGAA
    9781 GTAAGTTGGC CGCAGTGTTA TCACTCATGG TTATGGCAGC ACTGCATAAT TCTCTTACTG
    9841 TCATGCCATC CGTAAGATGC TTTTCTGTGA CTGGTGAGTA CTCAACCAAG TCATTCTGAG
    9901 AATAGTGTAT GCGGCGACCG AGTTGCTCTT GCCCGGCGTC AATACGGGAT AATACCGCGC
    9961 CACATAGCAG AACTTTAAAA GTGCTCATCA TTGGAAAACG TTCTTCGGGG CGAAAACTCT
  10021 CAAGGATOTT ACCGCTGTTG AGATCCAGTT CGATGTAACC CACTCGTGCA CCCAACTGAT
  10081 CTTCAGCATC TTTTACTTTC ACCAGCGTTT CTGGGTGAGC AAAAACAGGA AGGCAAAATG
  10141 CCGCAAAAAA GGGAATAAGG GCGACACGGA AATGTTGAAT ACTCATACTC TTCCTTTTTC
  10201 AATATTATTG AAGCATTTAT CAGGGTTATT GTCTCATGAG CGGATACATA TTTGAATGTA
  10261 TTTAGAAAAA TAAACAAATA GGGGTTCCGC GCACATTTCC CCGAAAAGTG CCACCTGGGT
  10321 CCTTTTCATC ACGTGCTATA AAAATAATTA TAATTTAAAT TTTTTAATAT AAATATATAA
  10381 ATTAAAAATA GAAAGTAAAA AAAGAAATTA AAGAAAAAAT AGTTTTTGTT TTCCGAAGAT
  10441 GTAAAAGACT CTAGGGGGAT CGCCAACAAA TACTACCTTT TATCTTGCTC TTCCTGCTCT
  10501 CAGGTATTAA TGCCGAATTG TTTCATCTTG TCTGTGTAGA AGACCACACA CGAAAATCCT
  10561 GTGATTTTAC ATTTTACTTA TCGTTAATCG AATGTATATC TATTTAATCT GCTTTTCTTG
  10621 TCTAATAAAT ATATATGTAA AGTACGCTTT TTGTTGAAAT TTTTTAAACC TTTGTTTATT
  10681 TTTTTTTCTT CATTCCGTAA CTCTTCTACC TTCTTTATTT ACTTTCTAAA ATCCAAATAC
  10741 AAAACATAAA AATAAATAAA CACAGAGTAA ATTCCCAAAT TATTCCATCA TTAAAAGATA
  10801 CGAGGCGCGT GTAAGTTACA GGCAAGCGAT CCGTCCTAAG AAACCA
(LANDING PAD_noCre)
SEQ ID NO: 6
          1 TTATTATCAT GACATTAACC TATAAAAATA GGCGTATCAC GAGGCCCTTT GTCACAGCTT
        61 GTCTGTAAGC GGATGCCGGG AGCAGACAAG CCCGTCAGGG CGCGTCAGCG GGTGTTGGCG
      121 GGTGTCGGGG CTGGCTTAAC TATGCGGCAT CAGAGCAGAT TGTACTGAGA GTGCACCATA
      181 CCACAGCTTT TCAATTCAAT TCATCATTTT TTTTTTATTC TTTTTTTTGA TTTCGGTTTC
      241 TTTGAAATTT TTTTGATTCG GTAATCTCCG AACAGAAGGA AGAACGAAGG AAGGAGCACA
      301 GACTTAGATT GGTATATATA CGCATATGTA GTGTTGAAGA AACATGAAAT TGCCCAGTAT
      361 TCTTAACCCA ACTGCACAGA ACAAAAACCT GCAGGAAACG AAGATAAATC ATGTCGAAAG
      421 CTACATATAA GGAACGTGCT GCTACTCATC CTAGTCCTGT TGCTGCCAAG CTATTTAATA
      481 TCATGCACGA AAAGCAAACA AACTTGTGTG CTTCATTGGA TGTTCGTACC ACCAAGGAAT
      541 TACTGGAGTT AGTTGAAGCA TTAGGTCCCA AAATTTGTTT ACTAAAAACA CATGTGGATA
      601 TCTTGACTGA TTTTTCCATG GAGGGCACAG TTAAGCCGCT AAAGGCATTA TCCGCCAAGT
      661 ACAATTTTTT ACTCTTCGAA GACAGAAAAT TTGCTGACAT TGGTAATACA GTCAAATTGC
      721 AGTACTCTGC GGGTGTATAC AGAATAGCAG AATGGGCAGA CATTACGAAT GCACACGGTG
      781 TGGTGGGCCC AGGTATTGTT AGCGGTTTGA AGCAGGCGGC AGAAGAAGTA ACAAAGGAAC
      841 CTAGAGGCCT TTTGATGTTA GCAGAATTGT CATGCAAGGG CTCCCTATCT ACTGGAGAAT
      901 ATACTAAGGG TACTGTTGAC ATTGCGAAGA GCGACAAAGA TTTTGTTATC GGCTTTATTG
      961 CTCAAAGAGA CATGGGTGGA AGAGATGAAG GTTACGATTG GTTGATTATG ACACCCGGTG
    1021 TGGGTTTAGA TGACAAGGGA GATGCATTGG GTCAACAGTA TAGAACCGTG GATGATGTGG
    1081 TGTCTACAGG ATCTGACATT ATTATTGTTG GAAGAGGACT ATTTGCAAAG GGAAGGGATG
    1141 CTAAGGTAGA GGGTGAACGT TACAGAAAAG CAGGCTGGGA AGCATATTTG AGAAGATGCG
    1201 GCCAGCAAAA CTAAAAAACT GTATTATAAG TAAATGCATG TATACTAAAC TCACAAATTA
    1261 GAGCTTCAAT TTAATTATAT CAGTTATTAC CCTATGCGGT GTGAAATACC GCACAGATGC
    1321 GTAAGGAGAA AATACCGCAT CAGGAAATTG TAAACGTTAA TATTTTGTTA AAATTCGCGT
    1381 TAAATTTTTG TTAAATCAGC TCATTTTTTA ACCAATAGGC CGAAATCGGC AAAATCCCTT
    1441 ATAAATCAAA AGAATAGACC GAGATAGGGT TGAGTGTTGT TCCAGTTTGG AACAAGAGTC
    1501 CACTATTAAA GAACGTGGAC TCCAACGTCA AAGGGCGAAA AACCGTCTAT CAGGGCGATG
    1561 GCCCACTACG TGAACCATCA CCCTAATCAA GTTTTTTGGG GTCGAGGTGC CGTAAAGCAC
    1621 TAAATCGGAA CCCTAAAGGG AGCCCCCGAT TTAGAGCTTG ACGGGGAAAG CCGGCGAACG
    1681 TGGCGAGAAA GGAAGGGAAG AAAGCGAAAG GAGCGGGCGC TAGGGCGCTG GCAAGTGTAG
    1741 CGGTCACGCT GCGCGTAACC ACCACACCCG CCGCGCTTAA TGCGCCGCTA CAGGGCGCGT
    1801 CGCGCCATTC GCCATTCAGG CTGCGCAACT GTTGGGAAGG GCGATCGGTG CGGGCCTCTT
    1861 CGCTATTACG CCAGCTGGCG AAAGGGGGAT GTGCTGCAAG GCGATTAAGT TGGGTAACGC
    1921 CAGGGTTTTC CCAGTCACGA CGTTGTAAAA CGACGGCCAG TGAGCGCGCG TAATACGACT
    1981 CACTATAGGG CGAATTGGGT ACCGCGAGAC CTTCCGGGTC TCAGAAGTTC CTATACTCTA
    2041 GAAAGAATAG GAACTTCGTC GACCCAGACA TGATAAGATA CATTGATGAG TTTGGACAAA
    2101 CCACAACTAG AATGCAGTGA AAAAAATGCT TTATTTGTGA AATTTGTGAT GCTATTGCTT
    2161 TATTTGTAAC CATTATAAGC TGCAATAAAC AAGTTAACAA CAACAATTGC ATTCATTTTA
    2221 TGTTTCAGGT TCAGGGGGAG GTGTGGGAGG TTTTTTAAAG CAAGTAAAAC CTCTACAAAT
    2281 GTGGTATGGC TGATTATGAT CTTAGTCCCC ATCCTCGAGC AGCCTCACCA TGGCCCCAGT
    2341 CTCAGAGTCC AGGTTTCTGA TGTAGTTCAT CACAATGTTC ACATTGGTCC AGCCACCAGC
    2401 CTGCATGATT TCAGGGATGG ACACACCAGC CCTGGCCATG TCCCTGGCAG CACCCACTCT
    2461 GGCAGAGTGG CCAGACCAGG CCAGGTATCT CTGCCCAGAG TCATCCTTGG CACCATAGAT
    2521 CAGGCGGTGG GTGGCCTCAA AGATCCCTTC CAGGGCCCGG GTGGACAGTT GGGAGGTGGC
    2581 AGAAGGGGCA GCCACACCAT TCTTTCTGAC CCGGCAGAAC AGGTAGTTGT TGGGGTCATC
    2641 AGCCACACCA GACACAGAGA TCCATCTCTC CACCAGCTTG GTAACCCCCA GGGACAGGGC
    2701 CTTCTCCACA CCAGCTGTGG ACACCAGGGT CTTGGTCCTG CCAATGTGGA TCAGCATTCT
    2761 CCCACCATCG GTGCGGGAGA TGTCCTTCAC TCTGATTCTG GCAATTTCGG CAATGCGCAG
    2821 CAGGGTGTTG TAGGCAATGC CCAGGAAGGC CAGGTTCCTG ATGTCCTGGC ATCTGTCAGA
    2881 GTTCTCCATC AGGGATCTGA CTTGGTCAAA GTCAGTGCGT TCAAAGGCCA GGGCCTGCTT
    2941 GGCTCTCTCC CCAGCATCCA CATTCTCCTT TCTGATTCTC CTCATCACCA GGGACACAGC
    3001 ATTGGAGTCA GAAGGGCGAG GCAGGCCAGA TCTCCTGTGC AGCATGTTGA GCTGGCCCAG
    3061 GTGCTGTTGG ATGGTCTTCA CAGCCAGGCC TCTGGCTTGC AGGTACAGGA GGTAGTCCCT
    3121 CACATCCTCA GGTTCAGCAG GGAACCATTT CCTGTTGTTC AGCTTGCACC AGGCAGCCCA
    3181 GGATCTGCAC ACAGACAGGA GCATCTTCCA GGTGTGTTCA GAGAAGGCCT GCCTGTCCCT
    3241 GAACATGTCC ATCAGGTTCT TCCTGACTTC ATCAGAGGTG GCATCCACAG GGAGGGCAGG
    3301 CAGGTTTTGG TGCACAGTCA GCAGGTTGGA GACTTTCCTC TTCTTCTTGG GCACCATGGT
    3361 GGCGAATTCT TTACGAGGGT AGGAAGTGGT ACGGAAAGTT GGTATAAGAC AAAAGTGTTG
    3421 TGGAATTGAA GTTTACTCAA AAAATCAGCA CTCTTTTATA GGCGCCCTGG TTTACATAAG
    3481 CAAAGCTTAT ACGTTCTCTA TCACTGATAG GGAGTAAACT GGATATACGT TCTCTATCAC
    3541 TGATAGGGAG TAAACTGTAG ATACGTTCTC TATCACTGAT AGGGAGTAAA CTGGTCATAC
    3601 GTTCTCTATC ACTGATAGGG AGTAAACTCC TTATACGTTC TCTATCACTG ATAGGGAGTA
    3661 AAGTCTGCAT ACGTTCTCTA TCACTGATAG GGAGTAAACT CTTCATACGT TCTCTATCAC
    3721 TGATAGGGAG TAAACTCGAG GGGATCCTGA AATTAACGCC GACGGGAGGG GOTTAATCCG
    3781 CAGCCTGGAG ATCCAGCCCC CTCAACCCGG GAGGTGGTCC CTGCAGTTAC GCCAATGATA
    3841 ACCCCCGCCA GAAAAATCTT AGTAGCCTTC CCTTTTTGTT TTCCGTGCCC CAACTCGGCG
    3901 GATTGACTCG GCCCCTTCCG GAAACACCCG AATCAACTTC TAGTCAAATT ATTGTTCACG
    3961 CCGCAATGAC CCACCCCTGG CCCGCGTCTG TGGAACTGAC CCCTGGTGTA CAGGAGAGTT
    4021 CGCTGCTGAA AGTGGTCCCA AAGGGGTACT AGTTTTTAAG CTCCCAACTC CCCCTCCCCC
    4081 AGCGTCTGGA GGATTCCACA CCCTCGCACC GCAGGGGCGA GGAAGTGGGC GGAGTCCGGT
    4141 TTTGGCGCCA GCCGCTGAGG CTGCCAAGCA GAAAAGCCAC CGCTGAGGAG ACTCCGGTCA
    4201 CTGTCCTCGC CCCGCCTCCC CCTTCCCTCC CCTTGGGGAC CACCGGGCGC CACGCCGCGA
    4261 ACGGTAAGTG CCGCGGTCGT CGGCGCCTCC GCCCTCCCCC TAGGGCCCCA ATTCCCAGCG
    4321 GGCGCGGCGC GCGGCCCCTC CCCCCGCCGG GCGCGCGCCC GCTGCCCCGC CCTTCGTGGC
    4381 CGCCCGGCGT GGGCGGTGCC ACCCCTCCCC CCGGCGGCCC CGCGCGCAGC TCCCGGCTCC
    4441 CTCCCCCTTC GGATGTGGCT TGAGCTGTAG GCGCGGAGGG CCGGAGACGC TGCAGACCCG
    4501 CGACCCGGAG CAGCTCGGAG GCGGTGAAGT CGGTGGCTTT CCTTCTCTCT AGCTCTCGCT
    4561 CGCTGGTGGT GCTTCAGATG CCACACGCGT CCCGGGGGCC CGGTTCTCCG CTCCCCTCGG
    4621 CTCCGGTGCC CGTCAGTGGG CAGAGCGCAC ATCGCCCACA GTCCCCGAGA AGTTGGGGGG
    4681 AGGGGTCGGC AATTGAACCG GTGCCTAGAG AAGGTGGCGC GGGGTAAACT GGGAAAGTGA
    4741 TGTCGTGTAC TGGCTCCGCC TTTTTCCCGA GGGTGGGGGA GAACCGTATA TAAGTGCAGT
    4801 AGTCGCCGTG AACGTTCTTT TTCGCAACGG GTTTGCCGCC AGAACACAGG TAAGTGCCGT
    4861 GTGTGGTTCC CGCGGGCCTG GCCTCTTTAC GGGTTATGGC CCTTGCGTGC CTTGAATTAC
    4921 TTCCACCTGG CTGCAGTACG TGATTCTTGA TCCCGAGCTT CGGGTTGGAA GTGGGTGGGA
    4981 GAGTTCGAGG CCTTGCGCTT AAGGAGCCCC TTCGCCTCGT GCATGAGTTG AGGCCTGGCC
    5041 TGGGCGCTGG GGCCGCCGCG TGCGAATCTG GTGGCACCTT CGCGCCTGTC TCGCTGCTTT
    5101 CGATAAGTCT CTAGCCATTT AAAATTTTTG ATGACCTGCT GCGACGCTTT TTTTCTGGCA
    5161 AGATAGTCTT GTAAATGCGG GCCAAGATCT GCACACTGGT ATTTCGGTTT TTGGGGCCGC
    5221 GGGCGGCGAC GGGGCCCGTG CGTCCCAGCG CACATGTTCG GCGAGGCGGG GCCTGCGAGC
    5281 GCGGCCACCG AGAATCGGAC GGGGGTAGTC TCAAGCTGGC CGGCCTGCTC TGGTGCCTGG
    5341 CCTCGCGCCG CCGTGTATCG CCCCGCCCTG GGCGGCAAGG CTGGCCCGGT CGGCACCAGT
    5401 TGCGTGAGCG GAAAGATGGC CGCTTCCCGG CCCTGCTGCA GGGAGCTCAA AATGGAGGAC
    5461 GCGGCGCTCG GGAGAGCGGG CGGGTGAGTC ACCCACACAA AGGAAAAGGG CCTTTCCGTC
    5521 CTCAGCCGTC GCTTCATGTG ACTCCACGGA GTACCGGGCG CCGTCCAGGC ACCTCGATTA
    5581 GTTCTCGAGC TTTTGGAGTA CGTCGTCTTT AGGTTGGGGG GAGGGGTTTT ATGCGATGGA
    5641 GTTTCCCCAC ACTGAGTGGG TGGAGACTGA AGTTAGGCCA GCTTGGCACT TGATGTAATT
    5701 CTCCTTGGAA TTTGCCCTTT TTGAGTTTGG ATCTTGGTTC ATTCTCAAGC CTCAGACAGT
    5761 GGTTCAAAGT TTTTTTCTTC CATTTCAGGT GTCGTGAGCC ACCATGGGAT CTAGACTGGA
    5821 CAAGAGCAAA GTCATAAACT CTGCTCTGGA ATTACTCAAT GGAGTCGGTA TCGAAGGCCT
    5881 GACGACAAGG AAACTCGCTC AAAAGCTGGG AGTTGAGCAG CCTACCCTGT ACTGGCACGT
    5941 GAAGAACAAG CGGGCCCTGC TCGATGCCCT GCCAATCGAG ATGCTGGACA GGCATCATAC
    6001 CCACTCCTGC CCCCTGGAAG GCGAGTCATG GCAAGACTTT CTGCGGAACA ACGCCAAGTC
    6061 ATACCGCTGT GCTCTCCTCT CACATCGCGA CGGGGCTAAA GTGCATCTCG GCACCCGCCC
    6121 AACAGAGAAA CAGTACGAAA CCCTGGAAAA TCAGCTCGCG TTCCTGTGTC AGCAAGGCTT
    6181 CTCCCTGGAG AACGCACTGT ACGCTCTGTC CGCCGTGGGC CACTTTACAC TGGGCTGCGT
    6241 ATTGGAGGAA CAGGAGCATC AAGTAGCAAA AGAGGAAAGA GAGACACCTA CCACCGATTC
    6301 TATGCCCCCA CTTCTGAAAC AAGCAATTGA GCTGTTCGAC CGGCAGGGAG CCGAACCTGC
    6361 CTTCCTTTTC GGCCTGGAAC TAATCATATG TGGCCTGGAG AAACAGCTAA AGTGCGAAAG
    6421 CGGCGGGCCG ACCGACGCCC TTGACGATTT TGACTTAGAC ATGCTCCCAG CCGATGCCCT
    6481 TGACGACTTT GACCTTGATA TGCTGCCTGC TGACGCTCTT GACGATTTTG ACCTTGACAT
    6541 GCTCCCCGGG GGCTCAGGCG CCACCAACTT TAGCCTGCTG AAGCAGGCAG GCGACGTCGA
    6601 GGAGAACCCC GGCCCTATAA CTTCGTATAG CATACATTAT ACGAAGTTAT CCAAAACATT
    6661 TAACATTTCT CAACAAGATC TAGAATTAGT AGAAGTAGCG ACAGAGAAGA TTACAATGCT
    6721 TTATGAGGAT AATAAACATC ATGTGGGAGC GGCAATTCGT ACGAAAACAG GAGAAATCAT
    6781 TTCGGCAGTA CATATTGAAG CGTATATAGG ACGAGTAACT GTTTGTGCAG AAGCCATTGC
    6841 GATTGGTAGT GCAGTTTCGA ATGGACAAAA GGATTTTGAC ACGATTGTAG CTGTTAGACA
    6901 CCCTTATTCT GACGAAGTAG ATAGAAGTAT TCGAGTGGTA AGTCCTTGTG GTATGTGTAG
    6961 GGAGTTGATT TCAGACTATG CACCAGATTG TTTTGTGTTA ATAGAAATGA ATGGCAAGTT
    7021 AGTCAAAACT ACGATTGAAG AACTCATTCC ACTCAAATAT ACCCGAAATG GCAGTGGAGA
    7081 GGGCAGAGGA AGTCTGCTAA CATGCGGTGA CGTCGAGGAG AATCCTGGCC CAGTGAGCAA
    7141 GGGCGAGGCA GTGATCAAGG AGTTCATGCG GTTCAAGGTG CACATGGAGG GCTCCATGAA
    7201 CGGCCACGAG TTCGAGATCG AGGGCGAGGG CGAGGGCCGC CCCTACGAGG GCACCCAGAC
    7261 CGCCAAGCTG AAGGTGACCA AGGGTGGCCC CCTGCCCTTC TCCTGGGACA TCCTGTCCCC
    7321 TCAGTTCATG TACGGCTCCA GGGCCTTCAC CAAGCACCCC GCCGACATCC CCGACTACTA
    7381 TAAGCAGTCC TTCCCCGAGG GCTTCAAGTG GGAGCGCGTG ATGAACTTCG AGGACGGCGG
    7441 CGCCGTGACC GTGACCCAGG ACACCTCCCT GGAGGACGGC ACCCTGATCT ACAAGGTGAA
    7501 GCTCCGCGGC ACCAACTTCC CTCCTGACGG CCCCGTAATG CAGAAGAAAA CAATGGGCTG
    7561 GGAAGCGTCC ACCGAGCGGT TGTACCCCGA GGACGGCGTG CTGAAGGGCG ACATTAAGAT
    7621 GGCCCTGCGC CTGAAGGACG GCGGCCGCTA CCTGGCGGAC TTCAAGACCA CCTACAAGGC
    7681 CAAGAAGCCC GTGCAGATGC CCGGCGCCTA CAACGTTGAC CGCAAGTTGG ACATCACCTC
    7741 CCACAACGAG GACTACACCG TGGTGGAACA GTACGAACGC TCCGAGGGCC GCCACTCCAC
    7801 CGGCGGCATG GACGAGCTGT ACAAGTGACC TCGACTGTGC CTTCTAGTTG CCAGCCATCT
    7861 GTTGTTTGCC CCTCCCCCGT GCCTTCCTTG ACCCTGGAAG GTGCCACTCC CACTGTCCTT
    7921 TCCTAATAAA ATGAGGAAAT TGCATCGCAT TGTCTGAGTA GGTGTCATTC TATTCTGGGG
    7981 GGTGGGGTGG GGCAGGACAG CAAGGGGGAG GATTGGGAAG ACAATAGCAG GCATGCTGGG
    8041 GATGCGGTGG GCTCTATGGC ATGCACAACT TCGTATAGAA AGGTATATAC GAAGTTGTAG
    8101 AGACCAACCG GGTCTCAGCT AGCTCCCTTT AGTGAGGGTT AATTGCGCGC TTGGCGTAAT
    8161 CATGGTCATA GCTGTTTCCT GTGTGAAATT GTTATCCGCT CACAATTCCA CACAACATAG
    8221 GAGCCGGAAG CATAAAGTGT AAAGCCTGGG GTGCCTAATG AGTGAGGTAA CTCACATTAA
    8281 TTGCGTTGCG CTCACTGCCC GCTTTCCAGT CGGGAAACCT GTCGTGCCAG CTGCATTAAT
    8341 GAATCGGCCA ACGCGCGGGG AGAGGCGGTT TGCGTATTGG GCGCTCTTCC GCTTCCTCGC
    8401 TCACTGACTC GCTGCGCTCG GTCGTTCGGC TGCGGCGAGC GGTATCAGCT CACTCAAAGG
    8461 CGGTAATACG GTTATCCACA GAATCAGGGG ATAACGCAGG AAAGAACATG TGAGCAAAAG
    8521 GCCAGCAAAA GGCCAGGAAC CGTAAAAAGG CCGCGTTGCT GGCGTTTTTC CATAGGCTCC
    8581 GCCCCCCTGA CGAGCATCAC AAAAATCGAC GCTCAAGTCA GAGGTGGCGA AACCCGACAG
    8641 GACTATAAAG ATACCAGGCG TTTCCCCCTG GAAGCTCCCT CGTGCGCTCT CCTGTTCCGA
    8701 CCCTGCCGCT TACCGGATAC CTGTCCGCCT TTCTCCCTTC GGGAAGCGTG GCGCTTTCTC
    8761 ATAGCTCACG CTGTAGGTAT CTCAGTTCGG TGTAGGTCGT TCGCTCCAAG CTGGGCTGTG
    8821 TGCACGAACC CCCCGTTCAG CCCGACCGCT GCGCCTTATC CGGTAACTAT CGTCTTGAGT
    8881 CCAACCCGGT AAGACACGAC TTATCGCCAC TGGCAGCAGC CACTGGTAAC AGGATTAGCA
    8941 GAGCGAGGTA TGTAGGCGGT GCTACAGAGT TOTTGAAGTG GTGGCCTAAC TACGGCTACA
    9001 CTAGAAGGAC AGTATTTGGT ATCTGCGCTC TGCTGAAGCC AGTTACCTTC GGAAAAAGAG
    9061 TTGGTAGCTC TTGATCCGGC AAACAAACCA CCGCTGGTAG CGGTGGTTTT TTTGTTTGCA
    9121 AGCAGCAGAT TACGCGCAGA AAAAAAGGAT CTCAAGAAGA TCCTTTGATC TTTTCTACGG
    9181 GGTCTGACGC TCAGTGGAAC GAAAACTCAC GTTAAGGGAT TTTGGTCATG AGATTATCAA
    9241 AAAGGATOTT CACCTAGATC CTTTTAAATT AAAAATGAAG TTTTAAATCA ATCTAAAGTA
    9301 TATATGAGTA AACTTGGTCT GACAGTTACC AATGOTTAAT CAGTGAGGCA CCTATCTCAG
    9361 CGATCTGTCT ATTTCGTTCA TCCATAGTTG CCTGACTCCC CGTCGTGTAG ATAACTACGA
    9421 TACGGGAGGG CTTACCATCT GGCCCCAGTG CTGCAATGAT ACCGCGAGAT CCACGCTCAC
    9481 CGGCTCCAGA TTTATCAGCA ATAAACCAGC CAGCCGGAAG GGCCGAGCGC AGAAGTGGTC
    9541 CTGCAACTTT ATCCGCCTCC ATCCAGTCTA TTAATTGTTG CCGGGAAGCT AGAGTAAGTA
    9601 GTTCGCCAGT TAATAGTTTG CGCAACGTTG TTGCCATTGC TACAGGCATC GTGGTGTCAC
    9661 GCTCGTCGTT TGGTATGGCT TCATTCAGCT CCGGTTCCCA ACGATCAAGG CGAGTTACAT
    9721 GATCCCCCAT GTTGTGCAAA AAAGCGGTTA GCTCCTTCGG TCCTCCGATC GTTGTCAGAA
    9781 GTAAGTTGGC CGCAGTGTTA TCACTCATGG TTATGGCAGC ACTGCATAAT TCTCTTACTG
    9841 TCATGCCATC CGTAAGATGC TTTTCTGTGA CTGGTGAGTA CTCAACCAAG TCATTCTGAG
    9901 AATAGTGTAT GCGGCGACCG AGTTGCTCTT GCCCGGCGTC AATACGGGAT AATACCGCGC
    9961 CACATAGCAG AACTTTAAAA GTGCTCATCA TTGGAAAACG TTCTTCGGGG CGAAAACTCT
  10021 CAAGGATOTT ACCGCTGTTG AGATCCAGTT CGATGTAACC CACTCGTGCA CCCAACTGAT
  10081 CTTCAGCATC TTTTACTTTC ACCAGCGTTT CTGGGTGAGC AAAAACAGGA AGGCAAAATG
  10141 CCGCAAAAAA GGGAATAAGG GCGACACGGA AATGTTGAAT ACTCATACTC TTCCTTTTTC
  10201 AATATTATTG AAGCATTTAT CAGGGTTATT GTCTCATGAG CGGATACATA TTTGAATGTA
  10261 TTTAGAAAAA TAAACAAATA GGGGTTCCGC GCACATTTCC CCGAAAAGTG CCACCTGGGT
  10321 CCTTTTCATC ACGTGCTATA AAAATAATTA TAATTTAAAT TTTTTAATAT AAATATATAA
  10381 ATTAAAAATA GAAAGTAAAA AAAGAAATTA AAGAAAAAAT AGTTTTTGTT TTCCGAAGAT
  10441 GTAAAAGACT CTAGGGGGAT CGCCAACAAA TACTACCTTT TATCTTGCTC TTCCTGCTCT
  10501 CAGGTATTAA TGCCGAATTG TTTCATCTTG TCTGTGTAGA AGACCACACA CGAAAATCCT
  10561 GTGATTTTAC ATTTTACTTA TCGTTAATCG AATGTATATC TATTTAATCT GCTTTTCTTG
  10621 TCTAATAAAT ATATATGTAA AGTACGCTTT TTGTTGAAAT TTTTTAAACC TTTGTTTATT
  10681 TTTTTTTCTT CATTCCGTAA CTCTTCTACC TTCTTTATTT ACTTTCTAAA ATCCAAATAC
  10741 AAAACATAAA AATAAATAAA CACAGAGTAA ATTCCCAAAT TATTCCATCA TTAAAAGATA
  10801 CGAGGCGCGT GTAAGTTACA GGCAAGCGAT CCGTCCTAAG AAACCA
(LANDING PAD_noCreTet)
SEQ ID NO: 7
          1 TTATTATCAT GACATTAACC TATAAAAATA GGCGTATCAC GAGGCCCTTT GTCACAGCTT
        61 GTCTGTAAGC GGATGCCGGG AGCAGACAAG CCCGTCAGGG CGCGTCAGCG GGTGTTGGCG
      121 GGTGTCGGGG CTGGCTTAAC TATGCGGCAT CAGAGCAGAT TGTACTGAGA GTGCACCATA
      181 CCACAGCTTT TCAATTCAAT TCATCATTTT TTTTTTATTC TTTTTTTTGA TTTCGGTTTC
      241 TTTGAAATTT TTTTGATTCG GTAATCTCCG AACAGAAGGA AGAACGAAGG AAGGAGCACA
      301 GACTTAGATT GGTATATATA CGCATATGTA GTGTTGAAGA AACATGAAAT TGCCCAGTAT
      361 TCTTAACCCA ACTGCACAGA ACAAAAACCT GCAGGAAACG AAGATAAATC ATGTCGAAAG
      421 CTACATATAA GGAACGTGCT GCTACTCATC CTAGTCCTGT TGCTGCCAAG CTATTTAATA
      481 TCATGCACGA AAAGCAAACA AACTTGTGTG CTTCATTGGA TGTTCGTACC ACCAAGGAAT
      541 TACTGGAGTT AGTTGAAGCA TTAGGTCCCA AAATTTGTTT ACTAAAAACA CATGTGGATA
      601 TCTTGACTGA TTTTTCCATG GAGGGCACAG TTAAGCCGCT AAAGGCATTA TCCGCCAAGT
      661 ACAATTTTTT ACTCTTCGAA GACAGAAAAT TTGCTGACAT TGGTAATACA GTCAAATTGC
      721 AGTACTCTGC GGGTGTATAC AGAATAGCAG AATGGGCAGA CATTACGAAT GCACACGGTG
      781 TGGTGGGCCC AGGTATTGTT AGCGGTTTGA AGCAGGCGGC AGAAGAAGTA ACAAAGGAAC
      841 CTAGAGGCCT TTTGATGTTA GCAGAATTGT CATGCAAGGG CTCCCTATCT ACTGGAGAAT
      901 ATACTAAGGG TACTGTTGAC ATTGCGAAGA GCGACAAAGA TTTTGTTATC GGCTTTATTG
      961 CTCAAAGAGA CATGGGTGGA AGAGATGAAG GTTACGATTG GTTGATTATG ACACCCGGTG
    1021 TGGGTTTAGA TGACAAGGGA GATGCATTGG GTCAACAGTA TAGAACCGTG GATGATGTGG
    1081 TGTCTACAGG ATCTGACATT ATTATTGTTG GAAGAGGACT ATTTGCAAAG GGAAGGGATG
    1141 CTAAGGTAGA GGGTGAACGT TACAGAAAAG CAGGCTGGGA AGCATATTTG AGAAGATGCG
    1201 GCCAGCAAAA CTAAAAAACT GTATTATAAG TAAATGCATG TATACTAAAC TCACAAATTA
    1261 GAGCTTCAAT TTAATTATAT CAGTTATTAC CCTATGCGGT GTGAAATACC GCACAGATGC
    1321 GTAAGGAGAA AATACCGCAT CAGGAAATTG TAAACGTTAA TATTTTGTTA AAATTCGCGT
    1381 TAAATTTTTG TTAAATCAGC TCATTTTTTA ACCAATAGGC CGAAATCGGC AAAATCCCTT
    1441 ATAAATCAAA AGAATAGACC GAGATAGGGT TGAGTGTTGT TCCAGTTTGG AACAAGAGTC
    1501 CACTATTAAA GAACGTGGAC TCCAACGTCA AAGGGCGAAA AACCGTCTAT CAGGGCGATG
    1561 GCCCACTACG TGAACCATCA CCCTAATCAA GTTTTTTGGG GTCGAGGTGC CGTAAAGCAC
    1621 TAAATCGGAA CCCTAAAGGG AGCCCCCGAT TTAGAGCTTG ACGGGGAAAG CCGGCGAACG
    1681 TGGCGAGAAA GGAAGGGAAG AAAGCGAAAG GAGCGGGCGC TAGGGCGCTG GCAAGTGTAG
    1741 CGGTCACGCT GCGCGTAACC ACCACACCCG CCGCGCTTAA TGCGCCGCTA CAGGGCGCGT
    1801 CGCGCCATTC GCCATTCAGG CTGCGCAACT GTTGGGAAGG GCGATCGGTG CGGGCCTCTT
    1861 CGCTATTACG CCAGCTGGCG AAAGGGGGAT GTGCTGCAAG GCGATTAAGT TGGGTAACGC
    1921 CAGGGTTTTC CCAGTCACGA CGTTGTAAAA CGACGGCCAG TGAGCGCGCG TAATACGACT
    1981 CACTATAGGG CGAATTGGGT ACCGCGAGAC CTTCCTGGTC TCAGAAGTTC CTATACTCTA
    2041 GAAAGAATAG GAACTTCGTC GACCCAGACA TGATAAGATA CATTGATGAG TTTGGACAAA
    2101 CCACAACTAG AATGCAGTGA AAAAAATGCT TTATTTGTGA AATTTGTGAT GCTATTGCTT
    2161 TATTTGTAAC CATTATAAGC TGCAATAAAC AAGTTAACAA CAACAATTGC ATTCATTTTA
    2221 TGTTTCAGGT TCAGGGGGAG GTGGTCCCTG CAGTTACGCC AATGATAACC CCCGCCAGAA
    2281 AAATCTTAGT AGCCTTCCCT TTTTGTTTTC CGTGCCCCAA CTCGGCGGAT TGACTCGGCC
    2341 CCTTCCGGAA ACACCCGAAT CAACTTCTAG TCAAATTATT GTTCACGCCG CAATGACCCA
    2401 CCCCTGGCCC GCGTCTGTGG AACTGACCCC TGGTGTACAG GAGAGTTCGC TGCTGAAAGT
    2461 GGTCCCAAAG GGGTACTAGT TTTTAAGCTC CCAACTCCCC CTCCCCCAGC GTCTGGAGGA
    2521 TTCCACACCC TCGCACCGCA GGGGCGAGGA AGTGGGCGGA GTCCGGTTTT GGCGCCAGCC
    2581 GCTGAGGCTG CCAAGCAGAA AAGCCACCGC TGAGGAGACT CCGGTCACTG TCCTCGCCCC
    2641 GCCTCCCCCT TCCCTCCCCT TGGGGACCAC CGGGCGCCAC GCCGCGAACG GTAAGTGCCG
    2701 CGGTCGTCGG CGCCTCCGCC CTCCCCCTAG GGCCCCAATT CCCAGCGGGC GCGGCGCGCG
    2761 GCCCCTCCCC CCGCCGGGCG CGCGCCCGCT GCCCCGCCCT TCGTGGCCGC CCGGCGTGGG
    2821 CGGTGCCACC CCTCCCCCCG GCGGCCCCGC GCGCAGCTCC CGGCTCCCTC CCCCTTCGGA
    2881 TGTGGCTTGA GCTGTAGGCG CGGAGGGCCG GAGACGCTGC AGACCCGCGA CCCGGAGCAG
    2941 CTCGGAGGCG GTGAAGTCGG TGGCTTTCCT TCTCTCTAGC TCTCGCTCGC TGGTGGTGCT
    3001 TCAGATGCCA CACGCGTCCC GGGGGCCCGG TTCTCCGCTC CCCTCGGCTC CGGTGCCCGT
    3061 CAGTGGGCAG AGCGCACATC GCCCACAGTC CCCGAGAAGT TGGGGGGAGG GGTCGGCAAT
    3121 TGAACCGGTG CCTAGAGAAG GTGGCGCGGG GTAAACTGGG AAAGTGATGT CGTGTACTGG
    3181 CTCCGCCTTT TTCCCGAGGG TGGGGGAGAA CCGTATATAA GTGCAGTAGT CGCCGTGAAC
    3241 GTTCTTTTTC GCAACGGGTT TGCCGCCAGA ACACAGGTAA GTGCCGTGTG TGGTTCCCGC
    3301 GGGCCTGGCC TCTTTACGGG TTATGGCCCT TGCGTGCCTT GAATTACTTC CACCTGGCTG
    3361 CAGTACGTGA TTCTTGATCC CGAGCTTCGG GTTGGAAGTG GGTGGGAGAG TTCGAGGCCT
    3421 TGCGCTTAAG GAGCCCCTTC GCCTCGTGCA TGAGTTGAGG CCTGGCCTGG GCGCTGGGGC
    3481 CGCCGCGTGC GAATCTGGTG GCACCTTCGC GCCTGTCTCG CTGCTTTCGA TAAGTCTCTA
    3541 GCCATTTAAA ATTTTTGATG ACCTGCTGCG ACGCTTTTTT TCTGGCAAGA TAGTCTTGTA
    3601 AATGCGGGCC AAGATCTGCA CACTGGTATT TCGGTTTTTG GGGCCGCGGG CGGCGACGGG
    3661 GCCCGTGCGT CCCAGCGCAC ATGTTCGGCG AGGCGGGGCC TGCGAGCGCG GCCACCGAGA
    3721 ATCGGACGGG GGTAGTCTCA AGCTGGCCGG CCTGCTCTGG TGCCTGGCCT CGCGCCGCCG
    3781 TGTATCGCCC CGCCCTGGGC GGCAAGGCTG GCCCGGTCGG CACCAGTTGC GTGAGCGGAA
    3841 AGATGGCCGC TTCCCGGCCC TGCTGCAGGG AGCTCAAAAT GGAGGACGCG GCGCTCGGGA
    3901 GAGCGGGCGG GTGAGTCACC CACACAAAGG AAAAGGGCCT TTCCGTCCTC AGCCGTCGCT
    3961 TCATGTGACT CCACGGAGTA CCGGGCGCCG TCCAGGCACC TCGATTAGTT CTCGAGCTTT
    4021 TGGAGTACGT CGTCTTTAGG TTGGGGGGAG GGGTTTTATG CGATGGAGTT TCCCCACACT
    4081 GAGTGGGTGG AGACTGAAGT TAGGCCAGCT TGGCACTTGA TGTAATTCTC CTTGGAATTT
    4141 GCCCTTTTTG AGTTTGGATC TTGGTTCATT CTCAAGCCTC AGACAGTGGT TCAAAGTTTT
    4201 TTTCTTCCAT TTCAGGTGTC GTGAGCCACC ATGATAACTT CGTATAGCAT ACATTATACG
    4261 AAGTTATCCA AAACATTTAA CATTTCTCAA CAAGATCTAG AATTAGTAGA AGTAGCGACA
    4321 GAGAAGATTA CAATGCTTTA TGAGGATAAT AAACATCATG TGGGAGCGGC AATTCGTACG
    4381 AAAACAGGAG AAATCATTTC GGCAGTACAT ATTGAAGCGT ATATAGGACG AGTAACTGTT
    4441 TGTGCAGAAG CCATTGCGAT TGGTAGTGCA GTTTCGAATG GACAAAAGGA TTTTGACACG
    4501 ATTGTAGCTG TTAGACACCC TTATTCTGAC GAAGTAGATA GAAGTATTCG AGTGGTAAGT
    4561 CCTTGTGGTA TGTGTAGGGA GTTGATTTCA GACTATGCAC CAGATTGTTT TGTGTTAATA
    4621 GAAATGAATG GCAAGTTAGT CAAAACTACG ATTGAAGAAC TCATTCCACT CAAATATACC
    4681 CGAAATGGCA GTGGAGAGGG CAGAGGAAGT CTGCTAACAT GCGGTGACGT CGAGGAGAAT
    4741 CCTGGCCCAG TGAGCAAGGG CGAGGCAGTG ATCAAGGAGT TCATGCGGTT CAAGGTGCAC
    4801 ATGGAGGGCT CCATGAACGG CCACGAGTTC GAGATCGAGG GCGAGGGCGA GGGCCGCCCC
    4861 TACGAGGGCA CCCAGACCGC CAAGCTGAAG GTGACCAAGG GTGGCCCCCT GCCCTTCTCC
    4921 TGGGACATCC TGTCCCCTCA GTTCATGTAC GGCTCCAGGG CCTTCACCAA GCACCCCGCC
    4981 GACATCCCCG ACTACTATAA GCAGTCCTTC CCCGAGGGCT TCAAGTGGGA GCGCGTGATG
    5041 AACTTCGAGG ACGGCGGCGC CGTGACCGTG ACCCAGGACA CCTCCCTGGA GGACGGCACC
    5101 CTGATCTACA AGGTGAAGCT CCGCGGCACC AACTTCCCTC CTGACGGCCC CGTAATGCAG
    5161 AAGAAAACAA TGGGCTGGGA AGCGTCCACC GAGCGGTTGT ACCCCGAGGA CGGCGTGCTG
    5221 AAGGGCGACA TTAAGATGGC CCTGCGCCTG AAGGACGGCG GCCGCTACCT GGCGGACTTC
    5281 AAGACCACCT ACAAGGCCAA GAAGCCCGTG CAGATGCCCG GCGCCTACAA CGTTGACCGC
    5341 AAGTTGGACA TCACCTCCCA CAACGAGGAC TACACCGTGG TGGAACAGTA CGAACGCTCC
    5401 GAGGGCCGCC ACTCCACCGG CGGCATGGAC GAGCTGTACA AGTGACCTCG ACTGTGCCTT
    5461 CTAGTTGCCA GCCATCTGTT GTTTGCCCCT CCCCCGTGCC TTCCTTGACC CTGGAAGGTG
    5521 CCACTCCCAC TGTCCTTTCC TAATAAAATG AGGAAATTGC ATCGCATTGT CTGAGTAGGT
    5581 GTCATTCTAT TCTGGGGGGT GGGGTGGGGC AGGACAGCAA GGGGGAGGAT TGGGAAGACA
    5641 ATAGCAGGCA TGCTGGGGAT GCGGTGGGCT CTATGGCATG CACAACTTCG TATAGAAAGG
    5701 TATATACGAA GTTGTAGAGA CCAACCTGGT CTCAGCTAGC TCCCTTTAGT GAGGGTTAAT
    5761 TGCGCGCTTG GCGTAATCAT GGTCATAGCT GTTTCCTGTG TGAAATTGTT ATCCGCTCAC
    5821 AATTCCACAC AACATAGGAG CCGGAAGCAT AAAGTGTAAA GCCTGGGGTG CCTAATGAGT
    5881 GAGGTAACTC ACATTAATTG CGTTGCGCTC ACTGCCCGCT TTCCAGTCGG GAAACCTGTC
    5941 GTGCCAGCTG CATTAATGAA TCGGCCAACG CGCGGGGAGA GGCGGTTTGC GTATTGGGCG
    6001 CTCTTCCGCT TCCTCGCTCA CTGACTCGCT GCGCTCGGTC GTTCGGCTGC GGCGAGCGGT
    6061 ATCAGCTCAC TCAAAGGCGG TAATACGGTT ATCCACAGAA TCAGGGGATA ACGCAGGAAA
    6121 GAACATGTGA GCAAAAGGCC AGCAAAAGGC CAGGAACCGT AAAAAGGCCG CGTTGCTGGC
    6181 GTTTTTCCAT AGGCTCCGCC CCCCTGACGA GCATCACAAA AATCGACGCT CAAGTCAGAG
    6241 GTGGCGAAAC CCGACAGGAC TATAAAGATA CCAGGCGTTT CCCCCTGGAA GCTCCCTCGT
    6301 GCGCTCTCCT GTTCCGACCC TGCCGCTTAC CGGATACCTG TCCGCCTTTC TCCCTTCGGG
    6361 AAGCGTGGCG CTTTCTCATA GCTCACGCTG TAGGTATCTC AGTTCGGTGT AGGTCGTTCG
    6421 CTCCAAGCTG GGCTGTGTGC ACGAACCCCC CGTTCAGCCC GACCGCTGCG CCTTATCCGG
    6481 TAACTATCGT CTTGAGTCCA ACCCGGTAAG ACACGACTTA TCGCCACTGG CAGCAGCCAC
    6541 TGGTAACAGG ATTAGCAGAG CGAGGTATGT AGGCGGTGCT ACAGAGTTCT TGAAGTGGTG
    6601 GCCTAACTAC GGCTACACTA GAAGGACAGT ATTTGGTATC TGCGCTCTGC TGAAGCCAGT
    6661 TACCTTCGGA AAAAGAGTTG GTAGCTCTTG ATCCGGCAAA CAAACCACCG CTGGTAGCGG
    6721 TGGTTTTTTT GTTTGCAAGC AGCAGATTAC GCGCAGAAAA AAAGGATCTC AAGAAGATCC
    6781 TTTGATCTTT TCTACGGGGT CTGACGCTCA GTGGAACGAA AACTCACGTT AAGGGATTTT
    6841 GGTCATGAGA TTATCAAAAA GGATCTTCAC CTAGATCCTT TTAAATTAAA AATGAAGTTT
    6901 TAAATCAATC TAAAGTATAT ATGAGTAAAC TTGGTCTGAC AGTTACCAAT GOTTAATCAG
    6961 TGAGGCACCT ATCTCAGCGA TCTGTCTATT TCGTTCATCC ATAGTTGCCT GACTCCCCGT
    7021 CGTGTAGATA ACTACGATAC GGGAGGGCTT ACCATCTGGC CCCAGTGCTG CAATGATACC
    7081 GCGAGATCCA CGCTCACCGG CTCCAGATTT ATCAGCAATA AACCAGCCAG CCGGAAGGGC
    7141 CGAGCGCAGA AGTGGTCCTG CAACTTTATC CGCCTCCATC CAGTCTATTA ATTGTTGCCG
    7201 GGAAGCTAGA GTAAGTAGTT CGCCAGTTAA TAGTTTGCGC AACGTTGTTG CCATTGCTAC
    7261 AGGCATCGTG GTGTCACGCT CGTCGTTTGG TATGGCTTCA TTCAGCTCCG GTTCCCAACG
    7321 ATCAAGGCGA GTTACATGAT CCCCCATGTT GTGCAAAAAA GCGGTTAGCT CCTTCGGTCC
    7381 TCCGATCGTT GTCAGAAGTA AGTTGGCCGC AGTGTTATCA CTCATGGTTA TGGCAGCACT
    7441 GCATAATTCT CTTACTGTCA TGCCATCCGT AAGATGOTTT TCTGTGACTG GTGAGTACTC
    7501 AACCAAGTCA TTCTGAGAAT AGTGTATGCG GCGACCGAGT TGCTCTTGCC CGGCGTCAAT
    7561 ACGGGATAAT ACCGCGCCAC ATAGCAGAAC TTTAAAAGTG CTCATCATTG GAAAACGTTC
    7621 TTCGGGGCGA AAACTCTCAA GGATCTTACC GCTGTTGAGA TCCAGTTCGA TGTAACCCAC
    7681 TCGTGCACCC AACTGATCTT CAGCATCTTT TACTTTCACC AGCGTTTCTG GGTGAGCAAA
    7741 AACAGGAAGG CAAAATGCCG CAAAAAAGGG AATAAGGGCG ACACGGAAAT GTTGAATACT
    7801 CATACTCTTC CTTTTTCAAT ATTATTGAAG CATTTATCAG GGTTATTGTC TCATGAGCGG
    7861 ATACATATTT GAATGTATTT AGAAAAATAA ACAAATAGGG GTTCCGCGCA CATTTCCCCG
    7921 AAAAGTGCCA CCTGGGTCCT TTTCATCACG TGCTATAAAA ATAATTATAA TTTAAATTTT
    7981 TTAATATAAA TATATAAATT AAAAATAGAA AGTAAAAAAA GAAATTAAAG AAAAAATAGT
    8041 TTTTGTTTTC CGAAGATGTA AAAGACTCTA GGGGGATCGC CAACAAATAC TACCTTTTAT
    8101 CTTGCTCTTC CTGCTCTCAG GTATTAATGC CGAATTGTTT CATCTTGTCT GTGTAGAAGA
    8161 CCACACACGA AAATCCTGTG ATTTTACATT TTACTTATCG TTAATCGAAT GTATATCTAT
    8221 TTAATCTGCT TTTCTTGTCT AATAAATATA TATGTAAAGT ACGCTTTTTG TTGAAATTTT
    8281 TTAAACCTTT GTTTATTTTT TTTTCTTCAT TCCGTAACTC TTCTACCTTC TTTATTTACT
    8281 TTAAACCTTT GTTTATTTTT TTTTCTTCAT TCCGTAACTC TTCTACCTTC TTTATTTACT
    8341 TTCTAAAATC CAAATACAAA ACATAAAAAT AAATAAACAC AGAGTAAATT CCCAAATTAT
    8401 TCCATCATTA AAAGATACGA GGCGCGTGTA AGTTACAGGC AAGCGATCCG TCCTAAGAAA
    8461 CCA
(mini_Syn_HLA)
SEQ ID NO: 8
          1 GGGTCCTTTT CATCACGTGC TATAAAAATA ATTATAATTT AAATTTTTTA ATATAAATAT
        61 ATAAATTAAA AATAGAAAGT AAAAAAAGAA ATTAAAGAAA AAATAGTTTT TGTTTTCCGA
      121 AGATGTAAAA GACTCTAGGG GGATCGCCAA CAAATACTAC CTTTTATCTT GCTCTTCCTG
      181 CTCTCAGGTA TTAATGCCGA ATTGTTTCAT CTTGTCTGTG TAGAAGACCA CACACGAAAA
      241 TCCTGTGATT TTACATTTTA CTTATCGTTA ATCGAATGTA TATCTATTTA ATCTGCTTTT
      301 CTTGTCTAAT AAATATATAT GTAAAGTACG CTTTTTGTTG AAATTTTTTA AACCTTTGTT
      361 TATTTTTTTT TCTTCATTCC GTAACTCTTC TACCTTCTTT ATTTACTTTC TAAAATCCAA
      421 ATACAAAACA TAAAAATAAA TAAACACAGA GTAAATTCCC AAATTATTCC ATCATTAAAA
      481 GATACGAGGC GCGTGTAAGT TACAGGCAAG CGATCCGTCC TAAGAAACCA TTATTATCAT
      541 GACATTAACC TATAAAAATA GGCGTATCAC GAGGCCCTTT CGTCTCGCGC GTTTCGGTGA
      601 TGACGGTGAA AACCTCTGAC ACATGCAGCT CCCGGAGACG GTCACAGCTT GTCTGTAAGC
      661 GGATGCCGGG AGCAGACAAG CCCGTCAGGG CGCGTCAGCG GGTGTTGGCG GGTGTCGGGG
      721 CTGGCTTAAC TATGCGGCAT CAGAGCAGAT TGTACTGAGA GTGCACCATA AATTCCCGTT
      781 TTAAGAGOTT GGTGAGCGCT AGGAGTCACT GCCAGGTATC GTTTGAACAC GGCATTAGTC
      841 AGGGAAGTCA TAACACAGTC CTTTCCCGCA ATTTTCTTTT TCTATTACTC TTGGCCTCCT
      901 CTAGTACACT CTATATTTTT TTATGCCTCG GTAATGATTT TCATTTTTTT TTTTCCACCT
      961 AGCGGATGAC TCTTTTTTTT TCTTAGCGAT TGGCATTATC ACATAATGAA TTATACATTA
    1021 TATAAAGTAA TGTGATTTCT TCGAAGAATA TACTAAAAAA TGAGCAGGCA AGATAAACGA
    1081 AGGCAAAGAT GACAGAGCAG AAAGCCCTAG TAAAGCGTAT TACAAATGAA ACCAAGATTC
    1141 AGATTGCGAT CTCTTTAAAG GGTGGTCCCC TAGCGATAGA GCACTCGATC TTCCCAGAAA
    1201 AAGAGGCAGA AGCAGTAGCA GAACAGGCCA CACAATCGCA AGTGATTAAC GTCCACACAG
    1261 GTATAGGGTT TCTGGACCAT ATGATACATG CTCTGGCCAA GCATTCCGGC TGGTCGCTAA
    1321 TCGTTGAGTG CATTGGTGAC TTACACATAG ACGACCATCA CACCACTGAA GACTGCGGGA
    1381 TTGCTCTCGG TCAAGCTTTT AAAGAGGCCC TAGGGGCCGT GCGTGGAGTA AAAAGGTTTG
    1441 GATCAGGATT TGCGCCTTTG GATGAGGCAC TTTCCAGAGC GGTGGTAGAT CTTTCGAACA
    1501 GGCCGTACGC AGTTGTCGAA CTTGGTTTGC AAAGGGAGAA AGTAGGAGAT CTCTCTTGCG
    1561 AGATGATCCC GCATTTTCTT GAAAGCTTTG CAGAGGCTAG CAGAATTACC CTCCACGTTG
    1621 ATTGTCTGCG AGGCAAGAAT GATCATCACC GTAGTGAGAG TGCGTTCAAG GCTCTTGCGG
    1681 TTGCCATAAG AGAAGCCACC TCGCCCAATG GTACCAACGA TGTTCCCTCC ACCAAAGGTG
    1741 TTCTTATGTA GTGACACCGA TTATTTAAAG CTGCAGCATA CGATATATAT ACATGTGTAT
    1801 ATATGTATAC CTATGAATGT CAGTAAGTAT GTATACGAAC AGTATGATAC TGAAGATGAC
    1861 AAGGTAATGC ATCATTCTAT ACGTGTCATT CTGAACGAGG CGCGCTTTCC TTTTTTCTTT
    1921 TTGCTTTTTC TTTTTTTTTC TCTTGAACTC GACGGATCTA TGCGGTGTGA AATACCGCAC
    1981 AGATGCGTCC TAGGGCGGCC GCATAACTTC GTATAGCATA CATTATACGA AGTTATCCGG
    2041 AACCGAGTAC AAGCCCACGG TGCGCCTCGC CACCCGCGAC GACGTCCCCA GGGCCGTCCG
    2101 CACCCTCGCC GCCGCGTTCG CCGACTACCC CGCCACGCGC CACACCGTCG ATCCGGACCG
    2161 CCACATCGAG CGCGTGACCG AGCTGCAAGA ACTCTTCCTC ACGCGCGTCG GGCTCGACAT
    2221 CGGCAAGGTG TGGGTCGCGG ACGACGGAGC CGCTGTGGCG GTCTGGACCA CGCCGGAGAG
    2281 CGTCGAAGCG GGGGCGGTGT TCGCCGAGAT CGGCCCGCGC ATGGCCGAGT TGAGCGGTTC
    2341 CCGGCTGGCC GCGCAGCAAC AGATGGAGGG CCTCCTGGCT CCGCACCGGC CCAAGGAGCC
    2401 CGCGTGGTTC CTGGCCACCG TCGGAGTCTC GCCCGACCAC CAGGGCAAGG GTCTGGGCAG
    2461 CGCCGTCGTG CTCCCCGGAG TGGAGGCGGC CGAGCGCGCC GGGGTGCCCG CCTTCCTGGA
    2521 AACCTCCGCG CCCCGCAACC TCCCCTTCTA CGAGCGGCTC GGCTTCACCG TCACCGCCGA
    2581 CGTCGAGGTG CCCGAAGGAC CGCGCACCTG GTGCATGACC CGCAAGCCCG GTGCCGCATG
    2641 CCCCACGCTA CTGCGGGTTT ATATAGACGG TCCCCACGGG ATGGGGAAAA CCACCACCAC
    2701 GCAACTGCTG GTGGCCCTGG GTTCGCGCGA CGATATCGTC TATGTACCCG AGCCGATGAC
    2761 TTACTGGCGG GTGCTGGGGG CTTCCGAGAC AATCGCGAAC ATCTACACCA CACAACACCG
    2821 CCTCGACCAG GGTGAGATAT CGGCCGGGGA CGCGGCGGTG GTAATGACAA GCGCCCAGAT
    2881 AACAATGGGT ATGCCTTATG CCGTGACCGA CGCCGTTCTG GCTCCTCATA TCGGGGGGGA
    2941 GGCTGGTAGC TCACATGCCC CGCCCCCGGC CCTCACCCTC ATCTTCGACC GCCATCCCAT
    3001 CGCCGCCCTC CTGTGCTACC CGGCCGCGCG GTACCTTATG GGCAGCATGA CCCCCCAGGC
    3061 CGTGCTGGCG TTCGTGGCCC TCATCCCGCC GACCTTGCCC GGCACCAACA TCGTGCTTGG
    3121 GGCCCTGCCG GAGGACAGAC ACATCGACCG CCTGGCCAAA CGCCAGCGCC CCGGCGAGCG
    3181 GCTGGACCTG GCTATGCTGG CTGCGATTCG CCGCGTTTAC GGGCTACTTG CCAATACGGT
    3241 GCGGTATCTG CAGTGCGGCG GGTCGTGGCG GGAGGACTGG GGACAGCTTT CGGGGACGGC
    3301 CGTGCCGCCC CAGGGTGCCG AGCCCCAGAG CAACGCGGGC CCACGACCCC ATATCGGGGA
    3361 CACGTTATTT ACCCTGTTTC GGGCCCCCGA GTTGCTGGCC CCCAACGGCG ACCTGTATAA
    3421 CGTGTTTGCC TGGGCCTTGG ACGTCTTGGC CAAACGCCTC CGTTCCATGC ACGTCTTTAT
    3481 CCTGGATTAC GACCAATCGC CCGCCGGCTG CCGGGACGCC CTGCTGCAAC TTACCTCCGG
    3541 GATGGTCCAG ACCCACGTCA CCACCCCCGG CTCCATACCG ACGATATGCG ACCTGGCGCG
    3601 CACGTTTGCC CGCGAGATGG GGGAGGCTAA CGAGCTCGGC AGTGGAGAGG GCAGAGGAAG
    3661 TCTGCTAACA TGCGGTGACG TCGAGGAGAA TCCTGGCCCA AGCCAGTTCG ACATCCTGTG
    3721 CAAGACCCCC CCCAAGGTGC TGGTGCGGCA GTTCGTGGAG AGATTCGAGA GGCCCAGCGG
    3781 CGAGAAGATC GCCAGCTGTG CCGCCGAGCT GACCTACCTG TGCTGGATGA TCACCCACAA
    3841 CGGCACCGCC ATCAAGAGGG CCACCTTCAT GAGCTACAAC ACCATCATCA GCAACAGCCT
    3901 GAGCTTCGAC ATCGTGAACA AGAGCCTGCA GTTCAAGTAC AAGACCCAGA AGGCCACCAT
    3961 CCTGGAGGCC AGCCTGAAGA AGCTGATCCC CGCCTGGGAG TTCACCATCA TCCCTTACAA
    4021 CGGCCAGAAG CACCAGAGCG ACATCACCGA CATCGTGTCC AGCCTGCAGC TGCAGTTCGA
    4081 GAGCAGCGAG GAGGCCGACA AGGGCAACAG CCACAGCAAG AAGATGCTGA AGGCCCTGCT
    4141 GTCCGAGGGC GAGAGCATCT GGGAGATCAC CGAGAAGATC CTGAACAGCT TCGAGTACAC
    4201 CAGCAGGTTC ACCAAGACCA AGACCCTGTA CCAGTTCCTG TTCCTGGCCA CATTCATCAA
    4261 CTGCGGCAGG TTCAGCGACA TCAAGAACGT GGACCCCAAG AGCTTCAAGC TGGTGCAGAA
    4321 CAAGTACCTG GGCGTGATCA TTCAGTGCCT GGTGACAGAA ACCAAGACAA GCGTGTCCAG
    4381 GCACATCTAC TTTTTCAGCG CCAGAGGCAG GATCGACCCC CTGGTGTACC TGGACGAGTT
    4441 TCTGAGGAAC AGCGAGCCCG TGCTGAAGAG AGTGAACAGG ACCGGCAACA GCAGCAGCAA
    4501 CAAGCAGGAG TACCAGCTGC TGAAGGACAA CCTGGTGCGC AGCTACAACA AGGCCCTGAA
    4561 GAAGAACGCC CCCTACCCCA TCTTCGCTAT CAAGAACGGC CCTAAGAGCC ACATCGGCAG
    4621 GCACCTGATG ACCAGCTTTC TGAGCATGAA GGGCCTGACC GAGCTGACAA ACGTGGTGGG
    4681 CAACTGGAGC GACAAGAGGG CCTCCGCCGT GGCCAGGACC ACCTACACCC ACCAGATCAC
    4741 CGCCATCCCC GACCACTACT TCGCCCTGGT GTCCAGGTAC TACGCCTACG ACCCCATCAG
    4801 CAAGGAGATG ATCGCCCTGA AGGACGAAAC CAACCCCATC GAGGAGTGGC AGCACATCGA
    4861 GCAGCTGAAG GGCAGCGCCG AGGGCAGCAT CAGATACCCC GCCTGGAACG GCATCATCAG
    4921 CCAGGAGGTG CTGGACTACC TGAGCAGCTA CATCAACAGG CGGATCACCG GTTGCGTACG
    4981 CGGATCCTCT GCTGGAGACA TGAGAGOTGC CAACCTTTGG CCAAGCCCGC TCATGATCAA
    5041 ACGCTCTAAG AAGAACAGCC TGGCCTTGTC CCTGACGGCC GACCAGATGG TCAGTGCCTT
    5101 GTTGGATGCT GAGCCCCCCA TACTCTATTC CGAGTATGAT CCTACCAGAC CCTTCAGTGA
    5161 AGCTTCGATG ATGGGCTTAC TGACCAACCT GGCAGACAGG GAGCTGGTTC ACATGATCAA
    5221 CTGGGCGAAG AGGGTGCCAG GCTTTGTGGA TTTGACCCTC CATGATCAGG TCCACCTTCT
    5281 AGAATGTGCC TGGCTAGAGA TCCTGATGAT TGGTCTCGTC TGGCGCTCCA TGGAGCACCC
    5341 AGTGAAGCTA CTGTTTGCTC CTAACTTGCT CTTGGACAGG AACCAGGGAA AATGTGTAGA
    5401 GGGCATGGTG GAGATOTTCG ACATGCTGCT GGCTACATCA TCTCGGTTCC GCATGATGAA
    5461 TCTGCAGGGA GAGGAGTTTG TGTGCCTCAA ATCTATTATT TTGCTTAATT CTGGAGTGTA
    5521 CACATTTCTG TCCAGCACCC TGAAGTCTCT GGAAGAGAAG GACCATATCC ACCGAGTCCT
    5581 GGACAAGATC ACAGACACTT TGATCCACCT GATGGCCAAG GCAGGCCTGA CCCTGCAGCA
    5641 GCAGCACCAG CGGCTGGCCC AGCTCCTCCT CATCCTCTCC CACATCAGGC ACATGAGTAA
    5701 CAAAGGCATG GAGCATCTGT ACAGCATGAA GTGCAAGAAC GTGGTGCCCC TCTATGACCT
    5761 GCTGCTGGAG GCGGCGGACG CCCACCGCCT ACATGCGCCC ACTAGCCGTG GAGGGGCATC
    5821 CGTGGAGGAG ACGGACCAAA GCCACTTGGC CACTGCGGGC TCTACTTCAT CGCATTCCTT
    5881 GCAAAAGTAT TACATCACGG GGGAGGCAGA GGGTTTCCCT GCCACAGCTG GCTCCGGCGA
    5941 GGGCAGGGGA AGTCTTCTAA CATGCGGGGA CGTGGAGGAA AATCCCGGCC CAGTGAGCAA
    6001 GGGTGAGGAG GATAACATGG CCTCTCTCCC AGCGACACAT GAGTTACACA TCTTTGGCTC
    6061 CATCAACGGT GTGGACTTTG ACATGGTGGG TCAGGGCACC GGCAATCCAA ATGATGGTTA
    6121 TGAGGAGTTA AACCTGAAGT CCACCAAGGG CGACCTCCAG TTCTCCCCCT GGATTCTGGT
    6181 CCCTCATATC GGGTATGGCT TCCATCAGTA CCTGCCCTAC CCTGACGGGA TGTCGCCTTT
    6241 CCAGGCCGCC ATGGTAGATG GCTCTGGATA CCAAGTCCAT CGCACAATGC AGTTTGAAGA
    6301 TGGTGCCTCC CTTACTGTGA ACTACCGCTA CACCTACGAG GGAAGCCACA TCAAAGGAGA
    6361 GGCCCAGGTG AAGGGGACTG GTTTCCCTGC TGACGGTCCT GTGATGACCA ACTCGCTGAC
    6421 CGCTGCGGAC TGGTGCAGGT CGAAGAAGAC TTACCCCAAC GACAAAACCA TCATCAGTAC
    6481 CTTTAAGTGG AGTTACACCA CTGGAAATGG CAAGCGCTAC CGGAGCACTG CGCGGACCAC
    6541 CTACACCTTT GCCAAGCCAA TGGCGGCTAA CTATCTGAAG AACCAGCCGA TGTACGTGTT
    6601 CCGTAAGACG GAACTCAAGC ACTCCAAGAC CGAGCTGAAC TTCAAGGAGT GGCAAAAGGC
    6661 GTTTACCGAT GTGATGGGCA TGGACGAGCT GTACAAGTGA ATTCGATCAT AATCAGCCAT
    6721 ACCACATTTG TAGAGGTTTT ACTTGCTTTA AAAAACCTCC CACACCTCCC CCTGAACCTG
    6781 AAACATAAAA TGAATGCAAT TGTTGTTGTT AACTTGTTTA TTGCAGCTTA TAATGGTTAC
    6841 AAATAAAGCA ATAGCATCAC AAATTTCACA AATAAAGCAT TTTTTTCACT GCATTCTAGT
    6901 TGTGGTTTGT CCAAACTCAT CAATGTATCT TATCATGTCT GGCTCGAGGG AAGTTCCTAT
    6961 ACTCTAGAAA GAATAGGAAC TTCAAAGGAA GTGCTTCAGG GTGAAGGAAA ATGATACTAG
    7021 ATGGTGACCC AGATATACAG AAAGGAACAA TTAACAACAG AAATGATGCA CATACACATG
    7081 TCACATTCAC ACTCATTTTC TTAATTTCCT GAAGACATAT GGCTACTTGT TTAAAACAAA
    7141 AAGTATTACA CTGTATCATT GAGCTTATAA TATAGGTTGA TGTAATATAT ACAACAATAA
    7201 TAGCACAATG GTAGGTTACA AGAAACTACA CTGTTAGAAG TGTCCTTTAT TTTCTGGATG
    7261 CAGOTTAATA TAACCTGAAC TTCACTGTGA AAAGTCAAGG AATCGGGTTT CCATTCTTTG
    7321 CTATTGTGTA AAGAAATATA GCCAAAAGCC ACTAGGAGAA TTAAAACCAT AAACTAAAAA
    7381 ATGTTTATTT GACACATAAG AAAGTAGTAA AGGAGGAACA GAAACAAAAA GATATGAGAC
    7441 AAATTGAAAA CGTATAGCAA AATTGTAGAC CAAAACCCAA CCATTGCAAG TGAAGAAATG
    7501 ACATGACCTG AGTCACATTA GCAGGACTGC TGAGCACTGT GGGGAGAACA GATATGGGCA
    7561 GGAGGTGAGG GACAGTGTTA GTGCCACAAT TCAGGAGGGA AAGGGTGGCA GGGACTAACG
    7621 GGGGTGGTGT GAAGGATGAG AAGGGCAGAC AGAAGGGCTG GAGAGGCAGG AGGAGAGGAA
    7681 AAGGAGCAGG AGAAAGAATT CTAAAGCAGT GGAAGAGCCT GGCAGAGGGT TCTTTGCATT
    7741 CGGTATTTAA TACATTTTGT TGGACTTCCT AAAAACTAAT TGGCTCCTTA TGATTAAAAA
    7801 AAAAAGAGTT ACAAAAATAC CAAGTGTTCA GATAAAATAT GCACACTGCT TGGATGTGCA
    7861 GAGTTCAGGA AAACAGGCAG TGCTTGAGCG CTGGTGAAGA GCATTGGGAC TGCATGGAGC
    7921 CCTCGCAACT TTGAGGTGAT GACTACAGGT TCCCGGTTGC AATAGACAGT AACAAACCCT
    7981 GCTTCTTTAT ATTCAGGAGA TGTTCTGGAC TCACACAGGG AAACTCAGGG TGGGGAATGA
    8041 AGATAATTTT AAATGCAACA ACCCAGAGTC ACAGATCCAT AGTCTGGGAA AGTAAAACTT
    8101 AGGAGCTTTG AGAGTTTAAT TGTAATGCTG TTTTGACACA GGTCTTTTGC AGATTGGAAT
    8161 TCTAATCATT CAGGGATTAC CAATATTGTG CTACCTACTG TATTAATAAA CAAAAAGGAA
    8221 ACTGGTCTCT ATGAGAATCT CTGTGTGGTG GCTTCAGACA AAACTTCGCC AGGTTTAGAG
    8281 AGAAAACCCC TGTCTCTACA CCTCCATTCC CAGGGCGAGC TCACTCTCTG GCATCAAGTT
    8341 CTCCGTGATC AGTTTCCCTA CACAAGANTC CAAGAGGAGA GGTAAGGAGT GAGAGGCAGG
    8401 GAGTCCAGTT CAGGGACAGG GATTCCAGGA GGAGAAGTGA AGGGGAAGCG GGTGGGCGCA
    8461 GCCTGGGGGT CTCTCCCTGG TTTCCACAGA CAGATCCTTG TGCAGGACTC AGGCAGACAG
    8521 TGTGACAAAG AGGCTTGGTG TAGGAGAAGA GGGATCAGGA CGAAGTCCCA TGTCCCGGAC
    8581 GGGGCTCTCA GGGTCTCAGG CTCCGAGGGC CGCGTCTGCA TTGGGGAGGC GCAGCGTTGG
    8641 GGATTCCCCA CTCCCACGAG TTTCACTTCT TCTCCCAACC TATGTCGGGT CCTTCTTCCA
    8701 GGATACTCGT GACGCGTCCC CATTTCCCAC TCCCATTGGG TGTCGGGTGT CTAGAGAAGC
    8761 CAATCAGTGT CGCCGGGGTC CCAGTTCTAA AGTCCCCACG CACCCACCCG GACTCAGAGT
    8821 CTCCTCAGAC ACCGAGATGC GGGTCACGGC ACCCCGAACC CTCCTCCTGC TGCTCTGGGG
    8881 GGCCCTGGCC CTGACCGAGA CCTGGGCGTG AGTGGCTTAA TGGAAAGGTG GTTTGGGCCT
    8941 CTGTGGGGAG GAGCGAGGGG ACCGCAGGCG GGGGCGCAGG ACCCGGGGAG CCGCGCCGGG
    9001 AGGAGGGTCT GGCGGGTCTC AGCCCCTCCT CGCCCCCAGG CTCCCACTCC ATGAGGTATT
    9061 TCTACACCGC CATGTCCCGG CCCGGCCGCG GGGAGCCCCG CTTCATCGCA GTGGGCTACG
    9121 TGGACGACAC GCAGTTCGTG AGGTTCGACA GCGACGCCGC GAGTCCGAGA GAGGAGCCGC
    9181 GGGCGCCGTG GATAGAGCAG GAGGGGCCGG AGTATTGGGA CCGGAACACA CAGATCTACA
    9241 AGGCCCAGGC ACAGACTGAC CGAGAGAGCC TGCGGAACCT GCGCGGCTAC TACAACCAGA
    9301 GCGAGGCCGG TGAGTGACCC CGGCCCGGGG CGCAGGTCAC GACTCCCCAT CCCCCACGTA
    9361 CGGCCCGGGT CGCCCCGAGT CTCCGGGTCC GAGATCCGCC TCCCTGAGGC CACGGGACCC
    9421 GCCCAGACCC TCGACCGGCG AGAGCCCCAG GCGCGTTTAC CCGGTTTCAT TTTCAGTTGA
    9481 GGCCAAAATC CCCGCGGGTT GGTCGGGGCG GGGCGGGGCT CGGGGGACGG GGCTGACCGC
    9541 GGGGCCGGGG CCAGGGTCTC ACACTTGGCA GACGATGTAT GGCTGCGACC TGGGGCCGGA
    9601 CGGGCGCCTC CTCCGCGGGC ATAACCAGTT AGCCTACGAC GGCAAGGATT ACATCGCCCT
    9661 GAACGAGGAC CTGAGCTCCT GGACCGCGGC GGACACCGCG GCTCAGATCA CCCAGCGCAA
    9721 GTGGGAGGCG GCCCGTGAGG CGGAGCAGCT GAGAGCCTAC CTGGAGGGCA CGTGCGTGGA
    9781 GTGGCTCCGC AGATACCTGG AGAACGGGAA GGAGACGCTG CAGCGCGCGG GTACCAGGGG
    9841 CAGTGGGGAG CCTTCCCCAT CTCCTATAGG TCGCCGGGGA TGGCCTCCCA CGAGAAGAGG
    9901 AGGAAAATGG GATCAGCGCT AGAATGTCGC CCTCCCTTGA ATGGAGAATG GCATGAGTTT
    9961 TCCTGAGTTT CCTCTGAGGG CCCCCTCTTC TCTCTAGGAC AATTAAGGGA TGACGTCTCT
  10021 GAGGAAATGG AGGGGAAGAC AGTCCCTAGA ATACTGATCA GGGGTCCTCT TTGACCCCTG
  10081 CAGCAGCCTT GGGAACTGCT ACAAAAGGCC AGAGCTTGTT CTCTGCCTCA CACTCAGTGT
  10141 GTTTGGGGCT CTGATTCCAG CACTTCTGAG TCACTTTACC TCCACTCAGA TCAGGAGCAG
  10201 AAGTCCCTGT TCCCCGCTCA GAGACTCGAA CTTTCCAATG AATAGGAGAT TATCCCAGGT
  10261 GCCTGCGTCC AGGCTGGTGT CTGGGTTCTG TGCCCCTTCC CCACCCCAGG TGTCCTGTCC
  10321 ATTCTCAGGC TGGTCACATG GGTGGTCCTA GGGTGTCCTA TGAGAGATGC AAAGCGCCTG
  10381 AATTTTCTGA CTCTTCCCAT CAGACCCCCC AAAGACACAC GTGACCCACC ACCCCATCTC
  10441 TGACCATGAG GCCACCCTGA GGTGCTGGGC CCTGGGCTTC TACCCTGCGG AGATCACACT
  10501 GACCTGGCAG CGGGATGGCG AGGACCAAAC TCAGGACACT GAGCTTGTGG AGACCAGACC
  10561 AGCAGGAGAT AGAACCTTCC AGAAGTGGGC AGCTGTGGTG GTGCCTTCTG GAGAAGAGCA
  10621 GAGATACACA TGCCATGTAC AGCATGAGGG GCTGCCGAAG CCCCTCACCC TGAGATGGGG
  10681 TAAGGAGGGG GATGAGGGGT CATATCTCTT CTCAGGGAAA GCAGGAGCCC TTCTGGAGCC
  10741 CTTCAGCAGG GTCAGGGCCC CTCGTCTTCC CCTCCTTTCC CAGAGCCATC TTCCCAGTCC
  10801 ACCATCCCCA TCGTGGGCAT TGTTGCTGGC CTGGCTGTCC TAGCAGTTGT GGTCATCGGA
  10861 GCTGTGGTCG CTACTGTGAT GTGTAGGAGG AAGAGCTCAG GTAGGGAAGG GGTGAGGGGT
  10921 GGGGTCTGGG TTTTCTTGTC CCACTGGGGG TTTCAAGCCC CAGGTAGAAG TGTTCCCTGC
  10981 CTCATTACTG GGAAGCAGCA TCCACACAGG GGCTAATGCA GCCTGGGACC CTGTGTGCCA
  11041 GCACTTACTC TCTTGTGCAG CACATGTGAC AATGAAGGAC GGATGTATCA CCTTGATGGT
  11101 TGTGGTGTTG GGGTCCTGAT TTCAGCATTC ATGAGTCAGG GGAAGGTCCC TGCTAAGGAC
  11161 AGACCTTAGG AGGGCAGTTG GTCCAGGACC CACACTTGCT TTCCTCGTGT TTCCTGATCC
  11221 TGCCTTGGGT CTGTAGTCAT ACTTCTGGAA ATTCCTTTTG GGTCCAAGAC GAGGAGGTTC
  11281 CTCTAAGATC TCATGGCCCT GCTTCCTCCC AGTCCCCTCA CAGGACATTT TCTTCCCACA
  11341 GGTGGAAAAG GAGGGAGCTA CTCTCAGGCT GCGTGTAAGT GGTGGGGGTG GGAGTGTGGA
  11401 GGAGCTCACC CACCCCATAA TTCCTCCTGT CCCACGTCTC CTGCGGGCTC TGACCAGGTC
  11461 CTGTTTTTGT TCTACTCCAG CCAGCGACAG TGCCCAGGGC TCTGATGTGT CTCTCACAGC
  11521 TTGAAAAGGT GAGATTCTTG GGGTCTAGAG TGGGCGGGGG GGTGGCGGGG GGTGGGGAGG
  11581 GGGCAGAGGG GAAAGGCCTG GGTAATGGAG ATTCTTTGAT TGGGATGTTT CGCGTGTGTG
  11641 GTGGGCTGTT TAGAGTGTCA TCACTTACCA TGACTAACCA GAATTTGTTC ATGACTGTTG
  11701 TTTTCTGTAG CCTGAGACAG CTGTCTTGTG AGGGACTGAG ATGCAGGATT TCTTCACTCC
  11761 TCCCCTTTGT GACTTCAAGA GCCTCTGGCA TCTCTTTCTG CAAAGGCACC TGAATGTGTC
  11821 TGCGTCCCTG TTAGCATAAT GTGAGGAGGT GGAGAGACAG CCCACCCCCG TGTCCACTGT
  11881 GACCCCTGTT CCCATGCTTG CTTCTAGAGC TCCTTTTCTC ACACATCGCC CAGGGTGGCC
  11941 CCTGCACACG GGGGTCTCTG TGCATTCTGA GACAAATTTT CAGAGCCATT CACCTCCTGC
  12001 CCTGCTTCTA GAGCTCCTTT TCTGCTCTGC TCTCCTGCCC TCTCTCCCTG CCCTGGTTCT
  12061 AGTGATCTTG GTGCTGAATC CAATCCCAAC TCCTGAATCT GTAAAGCAGA GTCTAATTTA
  12121 GACTTACATT TGTCTGTGAA ATTGGACCCA TCATCAAGGA CTGTTCTTTC CTGAAGGGAG
  12181 AACCTGATTG TGTGCTGCAG TGTGCTGGGG CAGGGGGTGC GGGGAGGGGG TTGCTGTAGA
  12241 AAGAGGGATG GGGAGGGAAA GGAAGTGCTT CAGGGTGGCC TGAGGCTATG TTAATAAAGA
  12301 TACTGCCTTC AAAATAGGGA GGTGCTCTAC AGTGATCATT CATTCAACTG ACCTTTGTCA
  12361 TTGGCCAGAC ATAGGACAGA ATGGTTCTGC ATCTGGGGAA CACCACTGAA GTAAAATCAG
  12421 AAAAATCTCT GGCCTTTTGT AGCAGGCGCG CCGAAGCTTG CTCCTCAATT CCACATGCTT
  12481 ATCATAGATA TCAAGCAGTG CCCTTGCTGT CTGCCCATCT TTTCTGACCC TGGGACACCA
  12541 CCCTTTATTT ACTTTCCCCA CAACTCCTTG AAGGTCAAGC CCCCTTGGCT GATACTCTGG
  12601 GTTATCATAG TAAATGTGCC CTCTATCTTT TGCAGGTCAC TGGAAAGGAG GGTTCCTAAA
  12661 GCATTCACTC CTGACCCTGA GGGAGGTGGG TGCACTCACT CCAGGACTCA GGTTCACCAC
  12721 AGATAAGCAA AAAAGTCCTC ACATTCAAAA TTGTCCTGGG CCACATGCAG CCCACGAGCC
  12781 GCGGGTTGGA CAAGCCTGCA TCTAGAACGT ATGCACCATA ACCCCAGACA TTTTTCTGTT
  12841 TTTACTTTAA GGATGCTTTC TAAATGCAAA AGCAGTCATA TCCCTAGCAG ACAACAAATG
  12901 TCAGTTGAAT GAATGATCAC TGTAGAGCAC CTCCCTATTC TAAAGCCCAT ATCTTTATTA
  12961 ATGTAGCCAC AGGGCAAATG CTGTTTTGTG GCAGCTAAAG AACATAAGTT CACGTGGACA
  13021 TCCATGCCGC CAGTGCTTTT CTCACCAGGG CTGCTTGTGT GTCCTCCCTC CTCCCACACC
  13081 AACCCTCCTG CACACTTCAG CACAAAACTA TATATTTCTC TTCAGGAACA ATAACCCTAG
  13141 CCTTATGGGT ACAATTTTCC AACCACATAT GAATCTAAAT TAGACTCTGC TTTATAAATC
  13201 TATGAGTTTG GATTGGAGCC AGCACTAGGA TTACTACAAC TCAGAGCAGG AAGAAGAGTA
  13261 GGAGGGCAGA AGAGGAGTTC CAACAGAAAG TTAACCATGA TGAGAAACTA TGGGACCCCT
  13321 CCTCTCTGCA AATGTCACAA TCTGGTTCCT TTAAAAAGAT TGGAGAAAAA TCGAAAATAC
  13381 CATTAAGGAA AAAGCCCTGG GAGGAAGGCA AGACCTGGAC AGGTCTGAAA ATTTGTCTCT
  13441 TGATACCACA AGGAGATTTG TATGCAGGGA CCACCCTAGG TGACATCCAA CTCCCTGTGA
  13501 TCACAGGTCT TGGTGGGACA AGGTTCTACT GAAGGGCCAA GGACAATGGA GCAGCAAAGA
  13561 TGACCCAGGT GAGCTGTGAC CACATAAAGC CCATGGTGGC CAGAACAGAA ACTGGGCACA
  13621 GCCCCATCTA CTCTCCTCCC CTGCAACAAA TCAGCACAAG AAACACGTGG ACTCTGGAAG
  13681 GTTCTCATGT CTTCCATTTA TTTTGTCTCT CAAATTTTAG GAATCTTCTC CTTTAATTAA
  13741 CTCATCAACC TCTCATGGCA AGAATTTGAG AAAGTAAATT TATACTCAGG TTCTAATTTT
  13801 AATAGGGAAG GAAGAAGTTA CAGCTCAGTG CACCATGAAG TTGAGACAGA GATGGAGACA
  13861 CCTCAGCCCC ACCTCTCTGG AACAGGAAAG ATGATTGGGG AGGGAGCACA GGTCAGCGTG
  13921 GGAAGAGGGT CATGGTGGAC ATGGGGGTGG GGTGGTCTCC CCACCTCCTC ACATTATGCC
  13981 TACACGAACA CAGACACATG CAGGTGCCTT TGCAGAAACA AAGTCAGGGT TCTTCAAGTC
  14041 ACAAAGGGAA GGGCAGGAAC AACTCTTGCC TCTCAGTCCC ACACAAGGCA GCTGTCTCAC
  14101 ACTATAGAAA AAAATATTCA TGAACAAATT CATATCCATC ACAGTGAGGG GTCACACCTT
  14161 AAACAGCCCA TCGCATGCTC AATACATCCA ATGCAAAGAA ACCCCATAGC ACAGCTGTGT
  14221 CCACTGTTCC GCCCAACACC CAACACACAT TAGGTCCTCC AAGCTCTCAC CTTTACAAGC
  14281 TGTGAGAGAC ACATCAGAGC CCTGGGCACT GTCACTGCCT GGGGTAGAAC AAAAACAGAA
  14341 CCTGGTCAGA TCCCACAGAA GATGTGGCTA GAGGAGGAAT TGTGGGGTGG GTGAGCTCCC
  14401 CCATGGGCTC CCAAACACAA TATCCCAAGG ACCTCAGGCA TCAGCCTCCT TCATACTTAC
  14461 TTGCAGCCTG AGTGTAACTC CCTCCTTTTC TATCTGTGAG AAGAAAATGT CCTGTGAGAT
  14521 ACCAGAAAGG AGCCAGGGCC TTAAGGTCCT AGAGGAACCT CCTAGTCTTG GACCCCAGAG
  14581 AAGTTTCCAG AAATGTGTGA CTGCAGACCC AGGGCGGGAT CAGGAAACAT GAAGAAAGCA
  14641 GGTGTGGGTC CTGGACCAAT AGCCCTCCTG AGGTCTGTCC TCAGGGACCT TCCCCTGTGA
  14701 CTTGTGACTG CTGGGATCAG GTCCCATCAC CGCCGTAATC AAGGTGATAA ATCTGTCCTT
  14761 CATTTTAACA GGTGCTTTAC AAAAGAGTAA GTGCTGGCAC ACAGGGCCCA GGCTGGGTAG
  14821 GCCCATGATT GTGGATGGTG CTTCCCAGTA ATGAGACAGG GCACATTTCT AGCTGGGGCT
  14881 TGGAACCCTC AGTGAGACAA GAAATCTCAG ACCCCACCCT TCACCCCTTC TCCACCTGAG
  14941 CTCTTCCTCC TCCACATCAC GGCAGCGACC ACAGCTCCAG TGATCACAGC TCCAAGGAGA
  15001 ACCAGGCCAG CAATGATGCC CACGATGGGG ATGGTGGGCT GGGAAGACAG CTCTGGGAAA
  15061 AGAGGGGAAG GTGAGGGGCC CTGACCCTGC TAAAGGTCTC CAGAGAGGCT CCTGCTTTCC
  15121 CTAAGAGACA TGACACCCCC ATCTCCCTCC TTACCCCATC TCAGGGTGAG GGGCTTGGGC
  15181 AGACCCTCAT GCTGCACATG GCAGGTGTAT CTCTGCTCCT CTCCAGAAGG CACCACCACA
  15241 GCCGCCCACT TCTGGAAGGT TCCATCCCCT GCAGGCCTGG TCTCCACGAG CTCCGTGTCC
  15301 TGGGTCTGGT CCTCCCCATC CCGCTGCCAG GTCAGTGTGA TCTCCGCAGG GTAGAAGCCC
  15361 AGGGCCCAGC ACCTCAGGGT GGCCTCATGG TCAGAGATGG GGTGGTGGGT CATATGTGTC
  15421 TTGGGGGGGT CTGACGGGAA GAGTCAGAAA ATTCAGGCAT TTTGCATCTG TCATGGGACA
  15481 CTCCACCAGC ACGCATGTGG CCATCTTGAG AATGGACAGG ACACCCGGGA TGGGGAAGAG
  15541 AGCACAGAAC CCAGACACCA GCCTGGACAC AGGCACCTGG GATAATCTTC TATTCCCTGA
  15601 GAAGGGAACA GCGACTTCTG GTCCTGACCT GAGTGGAGGC TGAGGGACTC AGAAGTGCTG
  15661 GACTCAGACC CCCACACACA TTGAGTGTGA AGCAGAGAAC AAGGCCTGAG AGGAAAAGTC
  15721 ACGGGCCCAA GGCTGCTGCC GGTGTCAAAG GGAACCACTC ATCAGTATTC GAGGGATCGT
  15781 CTTCCCGTCA CTCCTTCAGA GATTTTATCC CTTAATTGTG TCAGAGAGCA GGGCGGAACC
  15841 TCAGAGTCAC TCTCTGGTAC AGGATCTGGA ACCCAGGAGG ATTCCTCTCC CTCAGGACCA
  15901 GAGGGAGGGT GATATTCTAG TGTTGGTCCC AATTGTCTCC CCTCCTTGTG GGAGGCCAGC
  15961 CCGGGAGATC TACAGGCGAT CAGGAAGGCG CCCCGTGGCC CCTGGTACCC GTGCGCTGCA
  16021 GCGTCTCCTT CCCGTTCTCC AGGTATCTGC GGAGCCACTC CACGCACGTG CCCTCCAGGT
  16081 AGGCTCTCAA CTGCTCCGCC CAACGGGCCG CCTCCCACTT GCGCTGGGTG ATCTGAGCCG
  16141 CCATGTCCGC CGCGGTCCAA GAGCGCAGGT CCTCGTTCAG GGCGATGTAA TCCTTGCCGT
  16201 CGTAGGCGTG CTGTTCATAC CCGCGGAGGA AGCGCCCGTC CGACCCCACG TCGCAGCCAT
  16261 ACATTATCTG GATGGTGTGA GAACCTGGCC CCGACCCCGC GGTCAGCCCA GTCCCCCGAG
  16321 CCCCGCCCAG CCCCGACCAA CCCGGGGGAA TTTTTGGCCT AAACTGAAAA TGAAACCGGG
  16381 TAAAGGCGCC TGGGCCTCTC CCGGGTCAAG GGTCTCGGGG TCCCGCGGCT TCGGGGTGGA
  16441 TCTCGGACCC GGAGACTGTG GGCGACCTGG CCCGTCCGTG GGGGATGAGG GGTCGTGACC
  16501 TGCGCCCCCG GCCGGGGTCA CTCACCGGCC TCGCTCTGGT TGTAGTAGCC GCGCAGGGTC
  16561 CCCAGGTCCA CTCGGTCAGT CTGTGACTGG GCCTTCACAT TCCGTGTCTC CTGGTCCCAA
  16621 TACTCAGGCC TCTCCTGCTC TATCCACGGC GCCCGCGGCT CCATCCTCTG GCTCGCGGCG
  16681 TCGCTGTCGA ACCGCACGAA CTGCGTGTCG TCCACGTAGC CCACTGCGAT GAAGCGGGGC
  16741 TCTCCACTGC CGGGCCGGGA CACGGATGTG GAGAAATACC TCATGGAGTG GGAGCCTGGG
  16801 GGCGAGCAGT GGCTGAGACC TGACCGACCC TCCTCCCGGC GCGGCTCCCC CGGTCCTGCG
  16861 CCCCCGCCAG GAGGGCCCCT TGCTTCTCCC CGCAGAGGCG GTTTCCCTCC CGACCCCGCA
  16921 CTCACCCGCC CAGGTCTGGG TCAGGGCCAG GGCCCCCGAG AGTAGCAGGA GGAGGGTTCG
  16981 GGGCGCCATG ACGGCCATCC TCGGCGTCTG GGGAGAATCT GAGTCCCGGT GGGTGCGTGC
  17041 GGGCTTTAGA ACAGCGACCG CGACGACACT GATTGGCTTC TCTGGAAACC CGACACCCAA
  17101 TGGGAGTGAG AACTGGGTCC GCGTCGTGAG TATCCAGGAT GAAGGACCCT ACGTAGGTTG
  17161 GGAGAGGGAG AAAAGAAACT GCGGAGTTGG GGAATCCCCA AGGCTGTGAC TCCCCAATCC
  17221 ATACACCGCC TTCGGGGCCT GAGACCCTGA GAGCCACGCC TGGGGCCCTG GGACTTCGCC
  17281 CTGACCCCTC TGCTCCTGTG CGAAGCGCTC TGTCTCAATG TCTCCCTGAG TCTTGGCCCA
  17341 AGAGCTGTCT GAGAAACAAG GGAGAAACCC TCGGTTCACT TTTCATCCCA TTATCTCTGT
  17401 CCCTGAACTG GACTCCCTGA CTCCCACTCC TTACCTGTCC ACCTGGACTC TTCTAGAAGA
  17461 AAAATCACCC CATGGAGTTT GGTGCCAGAG AGTAAGCTTG CTCTGGGAAT GGAGGTGTAG
  17521 ATTTTTGTTG TTGTTGTTTT TTTTTTAAT CCGGAAAAGT TGTGCCTGAG TGCATGAGAC
  17581 AGAATAGAGA CCAATTTGCT CTTTGTTTAT TAACTACAGT GGGTAGCAGA ATCTTGGTAA
  17641 CCCCTGAATG ATCAGGAATC TAATAGGTAA AAAATGTGAC TTTGGCCCCT TAATGTATAA
  17701 ATGTGTCCAA AAGCATTACA ACAGGACTCA CAAAGCTACT AAGTTGCACT TTCCCAGACA
  17761 ATGTATCTGT GACTCCTGCT TGTCTTACTT TAAATTTGCC ATCATTCCTT AGCTCTGAGT
  17821 TTCTGTGTGA GTCCACGACA TCTCCTCAAT ACAAAGTAGC CACTGTGTTC CTATATGTTG
  17881 CAACCAGAAG CCATTACAGG CTTTATTCAC CTCAAAGTTG CAACTGTTCA AGGCAGTCAC
  17941 AATGCCCCTC ACTAGTGCTC ATGCACTGCC TGTTTTTAGG AAGTATCCAC TTCTAAGTGT
  18001 TGTGTATATT TTATAGGAAC ACTTAGTAAT TTTTAAACCT GATTAACATA AAAAATAGTT
  18061 TTTAGGCAGA CCCACATGAG GTATTAAAAG ACAACTGCCA AGGACACCCT GCTAGGCTCT
  18121 GTAGATTGAT GTATTAAAAT CCATAAAACA ATGTCTTTAA ACCTAAGAAT TCTGCTGCTT
  18181 TCAAATTCTT TCCTCTGCTC CTTTTCCTCA CCTCCTGCTT CTCCAGCCCT TCCCTCCATC
  18241 CTTCTCATCC TTCAGGCCCT CCTCTCCCCT TAGTCCCCAC CACACTGTCA CTCATGAATT
  18301 GTGACACTAG CACTGTCCCA TGACCTGCTA CGTAACTGTT CTTTTCACAG TGGCCCTGCT
  18361 CCTGTGAGTC GGAGTGTGTC ATTTCTCCAC TTAAAACACT CCAGTGTCTC CACCTCGGTC
  18421 TTGTGAAGCT TCTGGAGTGT CAGGCACTTG AGCATATGAG GGGATACCTC GTTCATTGTA
  18481 GGGACTAAGT TAATTTTTGT TGACTTAATG AATGAAATAT GAGTGTATTA AATTGCATCA
  18541 CAGATAATTA TAAACTGTAA AACACTGAAA AAGTTCAGAA AGATTTTATT TTATGCAACT
  18601 AGTGTGTATA TCAATTCATC AATTCATTCG CACACTACCA TGCCTGGCAA AACAACACAC
  18661 ATTTATCTGC TTATAGTTCC TTGGTCAGAA ATCTGGGCAA GATGTGGATA GAATCTCCAT
  18721 TCTGGGCTTC CAAAAGCTGT GTTTTCATTT TGAATCCTCC TTCGGGTTTA TACAGAGGTG
  18781 GCAGAATGCA GTTTCTTGCA GTTGTAAGAC TGAGGTCCCT GTTCCTGGCT AGCTGTCAAT
  18841 GTAGAGAACT GGGAGGGGCT CAATTCCCGG TGCTCACCAG CGTTCTTTCC TACACAGCCC
  18901 CTTAATTTTT CTTTTTTTTT TTTTTTTTGA GACGGAGTCT GGCTCTGTAG CCCAGGCTGG
  18961 GGTACAGTGG CCCAATGATC TCTGCTCACT ACAAGTTCTG CCTCCTGGGT TCACACCATT
  19021 CTCCTGTCTC AGTCCCAAGT AGCTGGGACT ACAGGCGCCC ACCACTATGC TCTAATTTTT
  19081 TTTTTCTTAG TAGAGACAGG GTTTCACCTT GTTAGCCAGG ATTGTCTCGA CCTCCTGACC
  19141 TCATGATCCG CCCGCCTCGG CCTCCCAAAG TGTCGGGATT ACAGGCGTGA GCCACCGCGC
  19201 CCGGCCCATA GCCCCTTCCT TTTCAAAGCC CATGGTGGAG GAAACCCCTC ATGCTGAGTC
  19261 CCTTTCACAC TGTAAGTCTC TATGCTCATG AAGAACCCAG TCCTTTCAAG GGCTCACCTG
  19321 ATTAGGACAG TCCAAGCAGG ATAAACCCAG CATAAAGTCA ACTAATTGAG ACCCTTAATT
  19381 ATATCTGCTA AATCCCTTCA CAGCAGCACC TACATTAGAG TTGGTTGAAT AACTGGAGGA
  19441 AGGTGAATGA CCAGGAGCTG CTTGTTGAGG CCATCATAGA ATCAGCCTAG CAAGGGTTGG
  19501 ATCTTCCTTT TGTGTTTAAT TGGGACACAG TTGGAAAGTG AAGTTCAAGT AACGTGATCA
  19561 TTGTGAATGG TCATAAAATA CATCCTCTTC AGCCATGGAA ATTGTCCTTA CTTTTTAAAA
  19621 CTAAGTTACA TGTTTAATAT CTTATAATTA ATTTAGGCCA GGTGTGGTGG CTCACATCTG
  19681 TCATCCTAGC ACTGTGGAAG GCAGAGGAAT GGAGATTTGT TGACTCCAGA AGTTCAAGAT
  19741 CAGCCTGGGC AAAACCTCCA TCTATACAAA AAATTAGAAA ATTAGCCAGG CATGGTAGTT
  19801 CATGATGGTA GTCGCAGCTA CTCAGGAGGC TGAGGTCAGA GAGTCCCTTG ACCCCAGGAG
  19861 TTCGACACTG CAGTGCATGG TGATCATGCC ACTGCATTCC AGCCTGGGTG ACAGAGCAAG
  19921 ACCCTGTCTC AAAAATAATA ATGATGATGA TGACACATTT AGAGCAAATG CAATTTGATG
  19981 TGTAATAATA CATCCTCTCT TGTGAAAATA TATTATTTAC TGTTGCATAA CAAATTACGT
  20041 AAAACTTAGC TCGAAACAAC AAATGTTTCT CATCTCCCAC AGTTTCCAAT CGTCAGGAAT
  20101 CCAGGAGAGG TTTCCCTGAG TGCTTCCTGC TCAGGGCCTC TCACAAGGTT GCACTCCAGT
  20161 TGTCAGCCAG GGGCCGCAGC ATCTGAGGGC TTCGCTGGGG CTGGGAATTC ACAAGAAACA
  20221 TGGATCAGTC ACATGGCTGT TGGAAAAGGC CTGGTTCCTT GTTGTCTGTT CCCAGAAGGC
  20281 CTCAGTTCTC AGTCATGTGG ACCTTCCTGC AGGGCTGCTT ATGGCACAGC GGCTGGCTTC
  20341 CCCAAGAGCT CATGATCCCA GATTCAGAGA GAGAGAAGGT GGAAGCCGCA GTTAGTTTTA
  20401 TGTTCTACAC CCAGAGTCAC AAACTGTTAT GTTGGCACTA TTCTATAAGT TAGAAGTTGT
  20461 ATTAGTCCAT TCTCACACTG CTATAAAGAA ATACCTGAGA CTGGGTAATT TATAAAGGAA
  20521 AGAGETTTAA TTGACCCACA GTTCTGCATG ACTGAGGAGG CTGCCCCAGG AAACTTACAG
  20581 TCATGGCAGA AGTGGAAGCA AACCCGTCCT TCTTCATGTG GTGACAGGAG AGAGAAATGC
  20641 ACAATGAAGC AGGGAAAAGC CCCTTATAAA ACCATCAGAT CTCATGAGTA TTCCCTCAGC
  20701 ATCAGGAGAA CAGCATGGGG GCACCGTCCC CATGATCCAA TCAACTCCCA TGAGGTCCCT
  20761 CCCCCAATAG GTAGGGATTA CAATTTGCAT AACAACTCAA GATGACATTT GGGTGGGAAC
  20821 ACAGAACCAG GCCATTTCAG AAGTGCCTCA TTAAGTCCAA GCCACACTCA AGAGAGGGAA
  20881 TTAAGCTGCA CCTCCAGAAA GGAACAGTAT CAAAGGATTT GAATATATGT TAAAAGCAAA
  20941 ATTATAACTA TTGTTTCAGG TTTCTGAAAA TCAAAGTCTT CTTGTATCTA ATTATTTTTC
  21001 ATTAACTCTT TAAGCTTGTC TTTTAATTTA ATTTTTTTTT AAGTTCCAGG GTACATGTGT
  21061 AGGATGTGCA GGTTTGTTAC ATAGGTAAAT GTGTGCCATG TTGGTTTGCT GCACCTGTCA
  21121 ACTCATCACC TAGGTATTAA CCCTGGCATG CATTAGCTAT TTTTGCTAAT GATCCTCTCA
  21181 TCACCACCCT CCCCCAACAG GCCCCAGTAT GTGTTGTTCC TCTCCCTGTG TCCATCTGTT
  21241 CTCATTGCTC AGTTCCCAGT TATATATGAG AACATGTGGT GTTTGGTTTT CTGTTCCTGT
  21301 GTGAGTTTGC TGAGGATAAT GGCTTCCACC TTCATCCATA TCCCTGCAAA GGACTTGATC
  21361 TCCTTCCTTT TTATGGCTGC ATAATATTCC ATGGTGTATA TGTACCATAT TTTCTTTATC
  21421 CAGACCATCA TTGATGGGCA TTTGGGTTGA TTCCATGTCT TTGCTATTGT GAATAGTGCT
  21481 GCCATGAATG TAAACATGCA TATGTCTTTA TAATAGAATG ATTTATATTC CTTTGGGTAT
  21541 ATACCCTGCA ATGGGATTGC TGGGTCAAAT GGTATTTCTG GTTCTAAATC TTTGAGGAAT
  21601 CGCCACACTG TCACTGTCTT CCACAATGGA TTAACCATTT ACTTTTCCAC CAACAGTGTA
  21661 AAAGCATTCC TATTTCTCCG CAACCTTGCC AGCATCTGTT GTTTCTTGAC TATTTAATAA
  21721 TTGCCATTCT GACTGGCATA AAATGGTATC TCATTTGTCG TTTTGATTTG CATTTCTCTA
  21781 ATGATCAGTG ATGGGAGCTT TCTTTTTCAT ATTTTTGTTG GCCACATGTA TGTCTTCTTT
  21841 AGAAGTGTCT GTTCATGTTC TTTGTCCACT TTTTAATGAA GTTTTTTTTC ATGTAAATTT
  21901 GCTTAAGTTC TTTGTGGATT CTGAATACTA GACTTTTGTC AGATGAACAG ATGGCAAAAT
  21961 TTTTCTCCCA TTCTGTAGCA TGTCTGTTCA CTCTGATTGA TGATAGTTTC TTTTGCTGTG
  22021 CAGAAAAAAA TTGCCTAATC TGATTGCTTT TCATTTACAA AAATGAGAAT CTCATACTTC
  22081 AGTTAACACA TCTAGCTTAG CATGATGCCA ATAATTTTGG CTTGATGCTG TAGCCCGAAA
  22141 TGGTTAGCTC GAATTGAGAG ACAAGTTATT TTCAAAAACT GGCTATCCGA TGGGCATTCT
  22201 ATAACACACT TAACATCTTT TGTTGTGGTA TTGAATGAGA TGTGATAGAA GTGATTTGGA
  22261 CACCACTATG ATTCTAAATA GCACTGCTAT TATGTGCTTC TGAATTTTTT TTCTTTTTAG
  22321 CAGTCCTATT CTGTTTCCAT TCATATTTGC TATTCCATCA CTGGGTATTC CTTCCTACTC
  22381 TTTTCATGTT TCATTTATTT TGATCAGTGA TGTGCATTTC CAATTCTGTA AAAGTTTAAT
  22441 TCAGTTGTAT GTGTGGTAAA ATGTAACATA GAATCTTTGC AAGATGAAAT TACCTTGCAC
  22501 AGCATATTGA ACATTGTATT ATTGAAAATC TAGAGAGATG CCAGTGAGCC AAGGATCGAA
  22561 TGACCTATAT GTAGCCAAGG CCGTTTTCAT GGCATCCTGG TCCTCTCTGT CACATAGCTC
  22621 CTTACAATTC TCTGTGGTTT TCTTCCAATG TAAGTAGTTC TTGTTAAGAA TCTTTGCAAT
  22681 GAACTTTGAA ATTCTTTTCT TTTTCATTGC TATAAAAAAC AGGTATATTT TAATTTGGAA
  22741 AACTCTGTTT AAGGAATTAT AATCAAGTAA CTACCAGGGG GCCCAGCACT GGTGTAAAAC
  22801 ACAAAACTGT AAAACTACCC TGGGCTGCTA CCCAGTACTC CCATTGCACA GGACAGTTTT
  22861 ACTTTCCCAC TTGAAAAGTG CATCTTTCAG ATGCCAGAGT GTTGTTCCAA TCTGTAAATC
  22921 TTTTGCAGAA CTAAACCAGT CAAGAAGTAC CATATACTCC ACATTCCCCC TCTTCTTCCA
  22981 TGTATCTATA GAATCATCTG GGAGGTGTGC TTCAATCCAG CTAGCAGTGA CTCCTGGACT
  23041 GATGGCTTTT CCAGGAACAC TGAGAGAATG TAAAATACAG GAATCCTGAT AATCCTGCAT
  23101 TCTTTGAGCA TCCATTATAA TCAAGCTGAT GTTTTTATCC ATCATCATTG TGTATAGTTC
  23161 CTTTGCTGTG ATTGCTCCTT TCTCTTTGGT CTCACATTTT TCATTCTTTT CACCATTGCT
  23221 CTTTTGGGTT TTGTCTTTGG AATCCAATAC AATCTCCAAA GAATCTTTAG CCAACGTGCT
  23281 GCCATCCTCT CTTCCTGTTT CCTGCCTTTT TTGTTGTAGC CACTGTGCTT CCCCCTGCCT
  23341 GTCCTTTTCC TCAAGTTTTT TCCAGATTTC AACTTCTTCA TATCTTAGTT TAAGGCTTTC
  23401 AGAGAGTCGT TCAGTTTCTC CAGTGGCTTT TTTGATGTTT GCAGGTCCAA GTATTGAATG
  23461 GAAGTAATAC TGCTGTTGCT TGAAATCAGT TCTTTTTTGG ATAGGATTAT AAACAGTCAT
  23521 GTTTCATATA CATGTGTGAG CTTTTTTTCA TATGTTTCTT GGCCACGTAA ATGTCTTCCT
  23581 CTGAGAAGTG TCTGTTTATA TCCTTTGCCC ACTTTTTGAT GGGGATGGGT TTTTTTCTTG
  23641 TAAATTTGTT TAAGTTCCTT GTAAACAAAC ATGTGAGCTC TCATCATTCT TGTTTAAACA
  23701 CCTAACAGTC ATCCTAACCA GTGCAACAAG GACCAGGCAT GGTGGCTCAT GCCTGTAATC
  23761 CCTGCATTTT GGGAGGCTGA GGTGGGAGGA TCACTTGAGA TCAGGAGTTT GAGACCAGCC
  23821 TGATAAGTGA GACCTCATCT CTACCAAATA AAAATAATTT TAAAAGAAAA TAGATCAATG
  23881 GATAAGAAAG GAAGAAATGA AAGTCTTTCT TTGTCACCAG CTTCATTGTA TATGTAGAAA
  23941 ACACTAGGGA ATTCTGAAAA AGTCTCTGGA ATTAATCATT GAATTTGCAA AATAGTTCAT
  24001 AAAATATATG TAATAAGTCG CTTAGATGAA CATGAAAAGA TAGCAAACAA TACTAGTCAT
  24061 CAAAGAAGTG CAAGTTAAAA CCACAATGAG AAACCATCAC ACATCACCTA GAGCAGGTAA
  24121 AGTTAAAAAG ACATATGATA AGTCTTAACA CTGGCAAGAA TATGGAAAAA ATAGGAATGT
  24181 CTGATATTGC TGGTAGGAAT GCAAAAAATG TGGCAGCCAA TTTGTAAAGT GGTATGGCAG
  24241 TTTCTTATAC AGTTACCCAT CTATTACCAC ATGGCCCAGC AATTCCACAA ATACGTATTT
  24301 ATCCAAAAGA AATAAAAATG TAAGTCCACA CTTGTAAGCA GTTATTTATA GTGGCTTCAT
  24361 TAATAACAAG CCCTAACTGG AGGAATCCAC ATGTCTATCA ACTGGAGACA CAGAAACCAA
  24421 TGAATAAACT GGGATTCCAG CAATACTCGG CAGCTGCTCA GCAACAAAAA TGAATGAATG
  24481 GCATCATCTC AAACATCGTT ATGCTAAGGG AGAGACCAAA CAAAAGACTA CATAACATAT
  24541 GATTGCATGT CCATGAAATT CTAGAAATGT CACTATTGCA GTGACAGAAA GCACAGCAGT
  24601 GGTTGAATGA AGGGAAGGGG GTGAGGGTGG GAGGCAAGGA TTAAATAGAA AGGGGCATAA
  24661 AGAAAGTTTT TAGGGAAAAG AAACTGTCCT CTACAGCGCC ACAACTCAGG AGTGACTGGG
  24721 AGGGGGAAGT AAGGGGAGAA GAAGGTCTGA GGGATAAGGG GCAGAGAGAA GGGCTGGGGA
  24781 AGCAGGAGGA GAGGACGAGG AGCAGGGGAA AGGACTCTAA AGCAGTGGAG GGGCCTAGTA
  24841 GGAGGATCTT TGCATTTGGT GTTTCTCTAC TGGGCAGTGT GGTAGTTACA CTATAAATAA
  24901 TTACCAATAT CCACCAAAAA GTGCAGCTAA AACTGGTGAA TTTTTTTACA CGTAAACGCC
  24961 CTGATAAGCA AAAAAAAAAA AAAAAAAAAA AAAAAAAAAA AAAAAAAAGG GGGGGGGGGA
  25021 GGAAAGAAGG CAAAAATAAA GACATTGTTA GATCATCAAA CCCATAAAAT TCATTTCCTA
  25081 GGGGTCCTGT ACTACATGTA ATTTTAAAGG ACGTTCTACA GGCTGAAGGG AAATGATACT
  25141 AGATGGTGAC CCAGATACAC AGAAAGGAAC AATTAACAAC AGAAATGATG CACATACACA
  25201 TATCACATAC ACACCCATTT TOTTAATTTC CTGAAGATAC GTGACTGCTT GTCTAAAACA
  25261 AAAAGTATTA CACTGTATTG TTGAGTTTAT AACGTATATT GATGTAACAT ATACAATAAT
  25321 AATAGCATAA TGATAGTTTA AATGAAACTA CACCATTTGA AGTGTCCTTT ATTTTGCTGG
  25381 ATGCAGOTTA ATATTACCTG AATTTCACTG TGAAAAGTCA AGGAATTGGG TTTCAATTCT
  25441 TACAACAATA AAAAATTAAT GTAAAGAAAT ATAGCTAAAA GCCACTAGGA GAATTAAAAC
  25501 CATAAGCTAA AAAATGTTTA CTTGAAACAT AAGAAAGTAG CAAAGGAGGA ACAGAAACAA
  25561 AAAGATATGA GACAAATTGA AAACGTATAG CAAAATGGTA GACCAAAATC CAACCATTAT
  25621 AAGTGAAGAA ATGACACGAC CTGAGTCACA TTAGCAGGAC TGCTGAGCAC TATGGGGAGA
  25681 ACAGACATGG GCAGGAAGTG AGGGACAGTG TTAGTGCCAC AATTCAGGAG TGACAGGGTG
  25741 GCGGGGACTA AAGGGGAAAG AGGGTGTGAG GGATGAGAGG GGCAGACAGA AGGGCTGGAG
  25801 AGGCAGGAGG TGAGGAAAAG GAGCAGAGGA AAGAATTCTA AAGCAGTAGA AGAGCCTGGC
  25861 AGGGGGTTCT TTGCATTCGG TATTTAATAC ATTTTGTGTG ACTGCCTTAA AACTAATGGG
  25921 CTCCTTATGA TTTTTTTTTA AAAAGGGGTT ACAAAAATAT CAAGTGTCCA AATAAAATAT
  25981 GCACACTGCT TAGATGTGCA TAGTTCCGGA AAACGGGCAG TGCTGGAGCG CTGGTGAAGA
  26041 GCATTGGGAC TGCATGGAGC CCTCACAACT TTGAGGTGAT GACTACAGGC TCCCGGTTGC
  26101 AATAGACAGT AACAAACCCT GCTTCTTTGT ATTCAGGAGA TGTTCTGGAC TCACACAGGG
  26161 AAACTCTGGC TAGAGAATGA GGATAACTTT AAATGCAACA ACCCAGAGTC ACAGATCCAT
  26221 AGTCTGCGAA AGTAAAACAG GAGCTTTGAG AATTTAATTG TAATGCAGTT TTGACACAGG
  26281 TCTTTCACAG ATTGGAATTC TAATCATTCA GGGATTACCA ATATTGTGCT ACCTACTGTA
  26341 TCAATAAACA AAAAGGAAAC TGGTCTCTAT GAGAATCTCT ACCTGGTGCT TTCAGACAAA
  26401 ACTTCACCAG GTTTAAAGAG AAAACTCCTG ACTCTACACG TCCATTCACA GGGCGAGCTC
  26461 ACTGTCTGGC ATCAAGTTCC CCATGGTGAG TTTCCCTGTA CAAGAGTCCA AGGGGAGAGG
  26521 TAAGTGTCCT TTATTTTGCT GGATGTAGTT TAATATTACC TGAGGTAAGG TAAGGTAAGG
  26581 CAAAGAGTGG GAGGCAGGGA GTCCAGTTCA GGGACGGGGA TTCCAGGAGG AGAAGTGAAG
  26641 GGGAAGGGGC TGGGCGCAGC CTGGGGGTCT CTCCCTGGTT TCCACAGACA GATCCTTGGC
  26701 CAGGACTCAG GCACACAGTG TGACAAAGAT GCTTGGTGTA GGAGAAGAGG AATCAGGACG
  26761 AAGTCCCAGG TCCCGGGCGG GGCTCTCAGG GTCTCAGGCT CCAAGGGCCG TGTCTGCACT
  26821 GGGGAGGCGC CGCGTTGAGG ATTCTCCACT CCCCTGAGTT TCACTTCTCC CAACCTGCGT
  26881 CGGGTCCTTC TTCCTGAATA CTCATGACGC GTCCCCAATT CCCACTCCCA TTGGGTGTCG
  26941 GATTCTAGAG AAGCCAATCA GCGTCTCCGC AGTCCCGGTT CTGAAGTCCC CAGTCACCCA
  27001 CCCGGACTCA GATTCTCCCC AGACGCCGAG ATGCGGGTCA TGGCGCCCCG AACCCTCATC
  27061 CTGCTGCTCT CGGGAGCCCT GGCCCTGACC GAGACCTGGG CCGGTGAGTG CGGGGTTGGG
  27121 AGGGAATCGG CCTCTGCGGA GAGGAGCGAG GGGCCCGCCC GGCGAGGGCG CAGGACCCGG
  27181 GGAGCCGCGC AGGGAGGAGG GTCGGGCGGG TCTCAGCCCC TCCTCGCCCC CAGGCTCCCA
  27241 CTCCATGAGG TATTTCTACA CCGCTGTGTC CCGGCCCGGC CGCGGGGAGC CCCACTTCAT
  27301 CGCAGTGGGC TACGTGGACG ACACGCAGTT CGTGCGGTTC GACAGCGACG CCGCGAGTCC
  27361 GAGAGGGGAG CCGCGGGCGC CGTGGGTGGA GCAGGAGGGG CCGGAGTATT GGGACCGGGA
  27421 GACACAGAAG TACAAGCGCC AGGCACAGAC TGACCGAGTG AGCCTGCGGA ACCTGCGCGG
  27481 CTACTACAAC CAGAGCGAGG CCAGTGAGTG ACCCCGGCCC GGGGCGCAGG TCACGACCCC
  27541 TCCTCATCCC CCACGGACGG CCCGGGTCGC CCCAAGTCTC CCGGTCTGAG ATCCACCCCG
  27601 AGGCTGNGCT GCGGAACCCG CCCAGACCCT CGACCGGAGA GAGCCCCAGT CACCTTTACC
  27661 CGGTTTCATT TTCAGTTTAG GCCAAAATCC CCGCGGGTTG GTCGGGGCGG GGCGGGGCTC
  27721 GGGGGACGGG GCTGACCGCG GGGGCGGGGC CAGGGTCTCA CATCATCCAG AGGATGTATG
  27781 GCTGCGACGT GGGGCCCGAC GGGCGCCTCC TCCGCGGGTA TGACCAGTAC GCCTACGACG
  27841 GCAAGGATTA CATCGCCCTG AACGAGGATC TGCGCTCCTG GACCGCCGCG GACACGGCGG
  27901 CTCAGATCAC CCAGCGCAAG TGGGAGGCGG CCCGTGAGGC GGAGCAGCTG AGAGCCTACC
  27961 TGGAGGGCCT GTGCGTGGAG TGGCTCCGCA GATACCTGAA GAATGGGAAG GAGACGCTGC
  28021 AGCGCGCGGG TACCAGGGGC AGTGGGGAGC CTTCCCCATC TCCTGTAGAT CTCCCGGGAT
  28081 GGCCTCCCAC GAGGAGGGGA GGAAAATGGG ATCAGCGCTA GAATATCGCC CTCCCTTGAA
  28141 TGGAGAATGG GATGAGTTTT CCTGAGTTTC CTCTGAGGGC CCCCTCTGCT CTCTAGGACA
  28201 ATTAAGGGAT GAAGTCCTTG AGGAAATGGA GGGGAAGACA GTCCCTAGAA TACTGATCAG
  28261 GGGTCCCCTT TGACCACTTT GACCACTGCA GCAGCTGTGG TCAGGCTGCT GACCTTTCTC
  28321 TCAGGCCTTG TTCTCTGCCT CACGCTCAAT GTGTTTGAAG GTTTGATTCC AGCTTTTCTG
  28381 AGTCCTTCGG CCTCCACTCA GGTCAGGACC AGAAGTCGCT GTTCCTCCCT CAGAGACTAG
  28441 AACTTTCCAA TGAATAGGAG ATTATCCCAG GTGCCTGTGT CCAGGCTGGC GTCTGGGTTC
  28501 TGTGCCCCCT TCCCCACCCC AGGTGTCCTG TCCGTTCTCA GGATGGTCAC ATGGGCGCTG
  28561 TTGGAGTGTC GCAAGAGAGA TACAAAGTGT CTGAATTTTC TGACTCTTCC CGTCAGAACA
  28621 CCCAAAGACA CACGTGACCC ACCATCCCGT CTCTGACCAT GAGGCCACCC TGAGGTGCTG
  28681 GGCCCTGGGC TTCTACCCTG CGGAGATCAC ACTGACCTGG CAGTGGGATG GGGAGGACCA
  28741 AACTCAGGAC ACTGAGCTTG TGGAGACCAG GCCAGCAGGA GATGGAACCT TCCAGAAGTG
  28801 GGCAGCTGTG GTGGTGCCTT CTGGAGAAGA GCAGAGATAC ACGTGCCATG TGCAGCACGA
  28861 GGGGCTGCCG GAGCCCCTCA CCCTGAGATG GGGTAAGGAG GGGGGGTGAG TCCTGNATGT
  28921 CTTCTCAGGG AAAGCAGAAG TCCTCCTGGA GCCCTTCAGC CAGGTCAGGG CTGAGGCTTG
  28981 GGGGTCAGGG CCCCTCACCT TCCCCTCCTT TCCCAGAGCC GTCTTCCCAG CCCACCATCC
  29041 CCATCGTGGG CATCGTTGCT GGCCTGGCTG TCCTGGCTGT CCTAGCTGTC CTAGGAGCTG
  29101 TGGTGGCTGT TGTGATGTGT AGGAGGAAGA GCTCAGGTAG GGAAGGGGTG AGGAGTGGGG
  29161 TCTGGGTTTT CTTGTTCCAC TGGGAGTTTC AAGCCCCAGG TAGAAGTGTG CCCCACCTCG
  29221 TTACTGGAAG CACCATCCAC ACATGGGCCA TCCCAGCCTG GGACCCTGTG TGCCAGCACT
  29281 TACTCTGTTG TGAAGCACAT GTGACAATGA AGGACAGATG TATCACCTTG ATGATTATGG
  29341 TGTTGGGGTC CTGATTCCAG CATTCATGAG TCAGGGGAAG GTCCCTGCTA AGGACAGACC
  29401 TTAGGAGGGC AGTTGCTCCA GAACCCACAG CTGCTTTCCC CGTGTTTCCT GATCCTGCCC
  29461 TGGGTCTGCA GTCATAGTTC TGGAAACTTC TCTTGGGTCC AAGACTAGGA GGTTCCCCTA
  29521 AGATCGCATG GCCCTGACTC CTCCCTGTCC CCTCACAGGG CATTTTCTTC CCACAGGTGG
  29581 AAAAGGAGGG AGCTGCTCTC AGGCTGCGTG GTAAGTGATG GCGGTGGGCG TGTGGAGGAG
  29641 CTCACCCACC CCATAATTCC TCTTGTCCCA CATCTCCTGC GGGCTCTGAC CAGGTCTTTT
  29701 TTTTTGTTCT ACCCCAGCCA GCAACAGTGC CCAGGGCTCT GATGAGTCTC TCATCGCTTG
  29761 TAAAGGTGAG ATTCTGGGGA GCTGAAGTGG TCGGGGGTGG GGCAGAGGGA AAAGGCCTAG
  29821 GTAATGGGGA TCCTTTGATT GGGACGTTTC GAATGTGTGG TGAGCTGTTC AGAGTGTGAT
  29881 CACTTACCAT GACTGACCTG AATTTGTTCA TGACTATTGT GTTCTGTAGC CTGAGACAGC
  29941 TGCCTGTGTG GGACTGAGAT GCAGGATTTC TTCACACCTC TCCTTTGTGA CTTCAAGAGC
  30001 CTCTGGCATC TCTTTCTGCA AAGGCATCTG AATGTGTCTG CGTTCCTGTT AGCATAATGT
  30061 GAGGAGGTGG ACAGACAGCC CACCCCCGTG TCCACCGTGA CCCCTGTCCC CACACTGACC
  30121 TGTGTTCCCT CCCCGATCAT CTTTCCTGTT CCAGAGAAGT GGGGCTGGAT GTCTCCATCT
  30181 CTGTCTCAAC TTCATGGTGC GCTGAGCTGC AACTTCTTAC TTCCCTAATG AAGTTAGGAA
  30241 CCTGAATATA AATTTGTTTT CTCAAATATT TGCTATGAAG GGTTGATGGA TTAATTAAAT
  30301 AAGTCAATTC CTGGAAGTTG AGAGAGCAAA TAAAGACCTG AGAACTTTCC AGAATCCGCA
  30361 TGTTCTCTGT GCTGAGTCTG TTGCAGGTGG GGGTGGGGAA GGCTGTGAGG AGCCGAGTGT
  30421 GGACGGGGCC TGTGCCTAGT TGCTGTTCAG TTCTTCATGG GCTTTATGTA GTCAGTCCTT
  30481 AGCTGGGTCA CCTTCACTGC TCCATTGTCC TTGTCCCTTC AGTGGAAACT TGTCCAGCAG
  30541 GAGCTGTGAC CACAGAGGCT CACACATCGC CCAGGGCAGC CCCTGCACAC GGGAGTCCCT
  30601 GTGCTTTCTG AGACAAATTT TCAGACCCAT TCAGCTCCTG CCCTCCTTAT AGGGCTCCTC
  30661 TTCTGCTTTG GTCTCCTGCC CTCTCTCCCT TCCCTGATTC CAGTGATCTT CGTGCTGACT
  30721 CCAATCCCAA CTCATGAATC TAAAGCAGAG CCTAATTTAG ATTTGTATTT GTTTGTAAAA
  30781 TTGGGTCCAT AGTCTAGAAT TGTTCCTTCC TGAAGAGAGA AACCTGATCG TGTGCTGCAG
  30841 CGTGCAGGGC GGTTGGTGTG GGAGGAGGGA TAGGGGAGGG AGGACACACA AGCAGCCCTG
  30901 CTGAGAAAAG TACAGGCGGC CTCGGTGTCA GTGTGAGGGG NCCTTGTGCT GCAGCTGCCA
  30961 CAAAACAGCA CTTGGCCTGA GGCTATGTTA ATAAAGATAC TGGCTTTAGA ATAGGAGGTG
  31021 CTCTACACTG ATCATTCAAC TGACCTTTGT TGTCAGCCAG ACACAGGACA GAAAAGTTCT
  31081 GCATCTGGGG AACACCATTG AAGTAAAATC AGAAAAATAT CTGAGCATAT GCTCCAGTGG
  31141 TAAGAGGCAG ACGATACATA CACTATAACC ACAGTAAGAA AAGAAAGTGA TGGAAGGTGG
  31201 TAAGTGCCAT GAGGCAGGTG ATCCGGGTAT GGGCAGTGGG GACAGGGAAG GTGGCTGTTG
  31261 GACAGGAGTT GTCAATGTGT GCCTTGTTGC AAAGATGACC TTTGAGGAAA GATTTGAGGG
  31321 ACATGAGGAT GTCTGGGGAA GTTCTTTCTA GGCAAGGAAA CACAACTTCG TATAGAAAGG
  31381 TATATACGAA GTTGTGCGGC CGCGTCGACC AATTCTCATG TTTGACAGCT TATCATCGAA
  31441 TTTCTGCCAT TCATCCGCTT ATTATCACTT ATTCAGGCGT AGCAACCAGG CGTTTAAGGG
  31501 CACCAATAAC TGCCTTAAAA AAATTAGAAA AACTCATCGA GCATCAAATG AAACTGCAAT
  31561 TTATTCATAT CAGGATTATC AATACCATAT TTTTGAAAAA GCCGTTTCTG TAATGAAGGA
  31621 GAAAACTCAC CGAGGCAGTT CCATAGGATG GCAAGATCCT GGTATCGGTC TGCGATTCCG
  31681 ACTCGTCCAA CATCAATACA ACCTATTAAT TTCCCCTCGT CAAAAATAAG GTTATCAAGT
  31741 GAGAAATCAC CATGAGTGAC GACTGAATCC GGTGAGAATG GCAAAAGATT ATGCATTTCT
  31801 TTCCAGACTT GTTCAACAGG CCAGCCATTA CGCTCGTCAT CAAAATCACT CGCATCAACC
  31861 AAACCGTTAT TCATTCGTGA TTGCGCCTGA GCGAGACGAA ATACGCGATC GCTGTTAAAA
  31921 GGACAATTAC AAACAGGAAT CGAATGCAAC CGGCGCAGGA ACACTGCCAG CGCATCAACA
  31981 ATATTTTCAC CTGAATCAGG ATATTCTTCT AATACCTGGA ATGCTGTTTT TCCGGGGATC
  32041 GCAGTGGTGA GTAACCATGC ATCATCAGGA GTACGGATAA AATGCTTGAT GGTCGGAAGA
  32101 GGCATAAATT CCGTCAGCCA GTTTAGTCTG ACCATCTCAT CTGTAACATC ATTGGCAACG
  32161 CTACCTTTGC CATGTTTCAG AAACAACTCT GGCGCATCGG GCTTCCCATA CAATCGATAG
  32221 ATTGTCGCAC CTGATTGCCC GACATTATCG CGAGCCCATT TATACCCATA TAAATCAGCA
  32281 TCCATGTTGG AATTTAATCG CGGCCTCGAG CAAGACGTTT CCCGTTGAAT ATGGCTCATT
  32341 TTAGCTTCCT TAGCTCCTGA AAATCTCGAT AACTCAAAAA ATACGCCCGG TAGTGATCTT
  32401 ATTTCATTAT GGTGAAAGTT GGAACCTCTT ACGTGCCGAT CAACGTCTCA TTTTCGCCAA
  32461 AAGTTGGCCC AGGGCTTCCC GGTATCAACA GGGACACCAG GATTTATTTA TTCTGCGAAG
  32521 TGATCTTCCG TCACAGGTAT TTATTCGCGA TAAGCTCATG GAGCGGCGTA ACCGTCGCAC
  32581 AGGAAGGACA GAGAAAGCGC GGATCTGGGA AGTGACGGAC AGAACGGTCA GGACCTGGAT
  32641 TGGGGAGGCG GTTGCCGCCG CTGCTGCTGA CGGTGTGACG TTCTCTGTTC CGGTCACACC
  32701 ACATACGTTC CGCCATTCCT ATGCGATGCA CATGCTGTAT GCCGGTATAC CGCTGAAAGT
  32761 TCTGCAAAGC CTGATGGGAC ATAAGTCCAT CAGTTCAACG GAAGTCTACA CGAAGGTTTT
  32821 TGCGCTGGAT GTGGCTGCCC GGCACCGGGT GCAGTTTGCG ATGCCGGAGT CTGATGCGGT
  32881 TGCGATGCTG AAACAATTAT CCTGAGAATA AATGCCTTGG CCTTTATATG GAAATGTGGA
  32941 ACTGAGTGGA TATGCTGTTT TTGTCTGTTA AACAGAGAAG CTGGCTGTTA TCCACTGAGA
  33001 AGCGAACGAA ACAGTCGGGA AAATCTCCCA TTATCGTAGA GATCCGCATT ATTAATCTCA
  33061 GGAGCCTGTG TAGCGTTTAT AGGAAGTAGT GTTCTGTCAT GATGCCTGCA AGCGGTAACG
  33121 AAAACGATTT GAATATGCCT TCAGGAACAA TAGAAATCTT CGTGCGGTGT TACGTTGAAG
  33181 TGGAGCGGAT TATGTCAGCA ATGGACAGAA CAACCTAATG AACACAGAAC CATGATGTGG
  33241 TCTGTCCTTT TACAGCCAGT AGTGCTCGCC GCAGTCGAGC GACAGGGCGA AGCCCTCGAG
  33301 CTGGTTGCCC TCGCCGCTGG GCTGGCGGCC GTCTATGGCC CTGCAAACGC GCCAGAAACG
  33361 CCGTCGAAGC CGTGTGCGAG ACACCGCGGC CGGCCGCCGG CGTTGTGGAT ACCTCGCGGA
  33421 AAACTTGGCC CTCACTGACA GATGAGGGGC GGACGTTGAC ACTTGAGGGG CCGACTCACC
  33481 CGGCGCGGCG TTGACAGATG AGGGGCAGGC TCGATTTCGG CCGGCGACGT GGAGCTGGCC
  33541 AGCCTCGCAA ATCGGCGAAA ACGCCTGATT TTACGCGAGT TTCCCACAGA TGATGTGGAC
  33601 AAGCCTGGGG ATAAGTGCCC TGCGGTATTG ACACTTGAGG GGCGCGACTA CTGACAGATG
  33661 AGGGGCGCGA TCCTTGACAC TTGAGGGGCA GAGTGCTGAC AGATGAGGGG CGCACCTATT
  33721 GACATTTGAG GGGCTGTCCA CAGGCAGAAA ATCCAGCATT TGCAAGGGTT TCCGCCCGTT
  33781 TTTCGGCCAC CGCTAACCTG TCTTTTAACC TGCTTTTAAA CCAATATTTA TAAACCTTGT
  33841 TTTTAACCAG GGCTGCGCCC TGTGCGCGTG ACCGCGCACG CCGAAGGGGG GTGCCCCCCC
  33901 TTCTCGAACC CTCCCGGTCG AGTGAGCGAG GAAGCACCAG GGAACAGCAC TTATATATTC
  33961 TGCTTACACA CGATGCCTGA AAAAACTTCC CTTGGGGTTA TCCACTTATC CACGGGGATA
  34021 TTTTTATAAT TATTTTTTTT ATAGTTTTTA GATCTTCTTT TTTAGAGCGC CTTGTAGGCC
  34081 TTTATCCATG CTGGTTCTAG AGAAGGTGTT GTGACAAATT GCCCTTTCAG TGTGACAAAT
  34141 CACCCTCAAA TGACAGTCCT GTCTGTGACA AATTGCCCTT AACCCTGTGA CAAATTGCCC
  34201 TCAGAAGAAG CTGTTTTTTC ACAAAGTTAT CCCTGCTTAT TGACTCTTTT TTATTTAGTG
  34261 TGACAATCTA AAAACTTGTC ACACTTCACA TGGATCTGTC ATGGCGGAAA CAGCGGTTAT
  34321 CAATCACAAG AAACGTTAAA ATAGCCCGCG AATCGTCCAG TCAAACGACC TCACTGAGGC
  34381 GGCATATAGT CTCTCCCGGG ATCAAAAACG TATGCTGTAT CTGTTCGTTG ACCAGATCAG
  34441 AAAATCTGAT GGCACCCTAC AGGAACATGA CGGTATCTGC GAGATCCATG TTGCTAAATA
  34501 TGCTGAAATA TTCGGATTGA CCTCTGCGGA AGCCAGTAAG GATATACGGC AGGCATTGAA
  34561 GAGTTTCGCG GGGAAGGAAG TGGTTTTTTA TCGCCCTGAA GAGGATGCCG GCGATGAAAA
  34621 AGGCTATGAA TCTTTTCCTT GGTTTATCAA ACGTGCGCAC AGTCCATCCA GAGGGCTTTA
  34681 CAGTGTACAT ATCAACCCAT ATCTCATTCC CTTCTTTATC GGGTTACAGA ACCGGTTTAC
  34741 GCAGTTTCGG CTTAGTGAAA CAAAAGAAAT CACCAATCCG TATGCCATGC GTTTATACGA
  34801 ATCCCTGTGT CAGTATCGTA AGCCGGATGG CTCAGGCATC GTCTCTCTGA AAATCGACTG
  34861 GATCATAGAG CGTTACCAGC TGCCTCAAAG TTACCAGCGT ATGCCTGACT TCCGCCGCCG
  34921 CTTCCTGCAG GTCTGTGTTA ATGAGATCAA CAGCAGAACT CCAATGCGCC TCTCATACAT
  34981 TGAGAAAAAG AAAGGCCGCC AGACGACTCA TATCGTATTT TCCTTCCGCG ATATCACTTC
  35041 CATGACGACA GGATAGTCTG AGGGTTATCT GTCACAGATT TGAGGGTGGT TCGTCACATT
  35101 TGTTCTGACC TACTGAGGGT AATTTGTCAC AGTTTTGCTG TTTCCTTCAG CCTGCATGGA
  35161 TTTTCTCATA CTTTTTGAAC TGTAATTTTT AAGGAAGCCA AATTTGAGGG CAGTTTGTCA
  35221 CAGTTGATTT CCTTCTCTTT CCCTTCGTCA TGTGACCTGA TATCGGGGGT TAGTTCGTCA
  35281 TCATTGATGA GGGTTGATTA TCACAGTTTA TTACTCTGAA TTGGCTATCC GCGTGTGTAC
  35341 CTCTACCTGG AGTTTTTCCC ACGGTGGATA TTTCTTCTTG CGCTGAGCGT AAGAGCTATC
  35401 TGACAGAACA GTTCTTCTTT GCTTCCTCGC CAGTTCGCTC GCTATGCTCG GTTACACGGC
  35461 TGCGGCGAGC GCTAGTGATA ATAAGTGACT GAGGTATGTG CTCTTCTTAT CTCCTTTTGT
  35521 AGTGTTGCTC TTATTTTAAA CAACTTTGCG GTTTTTTGAT GACTTTGCGA TTTTGTTGTT
  35581 GCTTTGCAGT AAATTGCAAG ATTTAATAAA AAAACGCAAA GCAATGATTA AAGGATGTTC
  35641 AGAATGAAAC TCATGGAAAC ACTTAACCAG TGCATAAACG CTGGTCATGA AATGACGAAG
  35701 GCTATCGCCA TTGCACAGTT TAATGATGAC AGCCCGGAAG CGAGGAAAAT AACCCGGCGC
  35761 TGGAGAATAG GTGAAGCAGC GGATTTAGTT GGGGTTTCTT CTCAGGCTAT CAGAGATGCC
  35821 GAGAAAGCAG GGCGACTACC GCACCCGGAT ATGGAAATTC GAGGACGGGT TGAGCAACGT
  35881 GTTGGTTATA CAATTGAACA AATTAATCAT ATGCGTGATG TGTTTGGTAC GCGATTGCGA
  35941 CGTGCTGAAG ACGTATTTCC ACCGGTGATC GGGGTTGCTG CCCATAAAGG TGGCGTTTAC
  36001 AAAACCTCAG TTTCTGTTCA TCTTGCTCAG GATCTGGCTC TGAAGGGGCT ACGTGTTTTG
  36061 CTCGTGGAAG GTAACGACCC CCAGGGAACA GCCTCAATGT ATCACGGATG GGTACCAGAT
  36121 CTTCATATTC ATGCAGAAGA CACTCTCCTG CCTTTCTATC TTGGGGAAAA GGACGATGTC
  36181 ACTTATGCAA TAAAGCCCAC TTGCTGGCCG GGGCTTGACA TTATTCCTTC CTGTCTGGCT
  36241 CTGCACCGTA TTGAAACTGA GTTAATGGGC AAATTTGATG AAGGTAAACT GCCCACCGAT
  36301 CCACACCTGA TGCTCCGACT GGCCATTGAA ACTGTTGCTC ATGACTATGA TGTCATAGTT
  36361 ATTGACAGCG CGCCTAACCT GGGTATCGGC ACGATTAATG TCGTATGTGC TGCTGATGTG
  36421 CTGATTGTTC CCACGCCTGC TGAGTTGTTT GACTACACCT CCGCACTGCA GTTTTTCGAT
  36481 ATGCTTCGTG ATCTGCTCAA GAACGTTGAT CTTAAAGGGT TCGAGCCTGA TGTACGTATT
  36541 TTGCTTACCA AATACAGCAA TAGCAATGGC TCTCAGTCCC CGTGGATGGA GGAGCAAATT
  36601 CGGGATGCCT GGGGAAGCAT GGTTCTAAAA AATGTTGTAC GTGAAACGGA TGAAGTTGGT
  36661 AAAGGTCAGA TCCGGATGAG AACTGTTTTT GAACAGGCCA TTGATCAACG CTCTTCAACT
  36721 GGTGCCTGGA GAAATGCTCT TTCTATTTGG GAACCTGTCT GCAATGAAAT TTTCGATCGT
  36781 CTGATTAAAC CACGCTGGGA GATTAGATAA TGAAGCGTGC GCCTGTTATT CCAAAACATA
  36841 CGCTCAATAC TCAACCGGTT GAAGATACTT CGTTATCGAC ACCAGCTGCC CCGATGGTGG
  36901 ATTCGTTAAT TGCGCGCGTA GGAGTAATGG CTCGCGGTAA TGCCATTACT TTGCCTGTAT
  36961 GTGGTCGGGA TGTGAAGTTT ACTCTTGAAG TGCTCCGGGG TGATAGTGTT GAGAAGACCT
  37021 CTCGGGTATG GTCAGGTAAT GAACGTGACC AGGAGCTGCT TACTGAGGAC GCACTGGATG
  37081 ATCTCATCCC TTCTTTTCTA CTGACTGGTC AACAGACACC GGCGTTCGGT CGAAGAGTAT
  37141 CTGGTGTCAT AGAAATTGCC GATGGGAGTC GCCGTCGTAA AGCTGCTGCA CTTACCGAAA
  37201 GTGATTATCG TGTTCTGGTT GGCGAGCTGG ATGATGAGCA GATGGCTGCA TTATCCAGAT
  37261 TGGGTAACGA TTATCGCCCA ACAAGTGCTT ATGAACGTGG TCAGCGTTAT GCAAGCCGAT
  37321 TGCAGAATGA ATTTGCTGGA AATATTTCTG CGCTGGCTGA TGCGGAAAAT ATTTCACGTA
  37381 AGATTATTAC CCGCTGTATC AACACCGCCA AATTGCCTAA ATCAGTTGTT GCTCTTTTTT
  37441 CTCACCCCGG TGAACTATCT GCCCGGTCAG GTGATGCACT TCAAAAAGCC TTTACAGATA
  37501 AAGAGGAATT ACTTAAGCAG CAGGCATCTA ACCTTCATGA GCAGAAAAAA GCTGGGGTGA
  37561 TATTTGAAGC TGAAGAAGTT ATCACTCTTT TAACTTCTGT GCTTAAAACG TCATCTGCAT
  37621 CAAGAACTAG TTTAAGCTCA CGACATCAGT TTGCTCCTGG AGCGACAGTA TTGTATAAGG
  37681 GCGATAAAAT GGTGCTTAAC CTGGACAGGT CTCGTGTTCC AACTGAGTGT ATAGAGAAAA
  37741 TTGAGGCCAT TOTTAAGGAA CTTGAAAAGC CAGCACCCTG ATGCGACCTC GTTTTAGTCT
  37801 ACGTTTATCT GTCTTTACTT AATGTCCTTT GTTACAGGCC AGAAAGCATA ACTGGCCTGA
  37861 ATATTCTCTC TGGGCCCACT GTTCCACTTG TATCGTCGGT CTGATAATCA GACTGGGACC
  37921 ACGGTCCCAC TCGTATCGTC GGTCTGATTA TTAGTCTGGG ACCACGGTCC CACTCGTATC
  37981 GTCGGTCTGA TTATTAGTCT GGGACCACGG TCCCACTCGT ATCGTCGGTC TGATAATCAG
  38041 ACTGGGACCA CGGTCCCACT CGTATCGTCG GTCTGATTAT TAGTCTGGGA CCATGGTCCC
  38101 ACTCGTATCG TCGGTCTGAT TATTAGTCTG GGACCACGGT CCCACTCGTA TCGTCGGTCT
  38161 GATTATTAGT CTGGAACCAC GGTCCCACTC GTATCGTCGG TCTGATTATT AGTCTGGGAC
  38221 CACGGTCCCA CTCGTATCGT CGGTCTGATT ATTAGTCTGG GACCACGATC CCACTCGTGT
  38281 TGTCGGTCTG ATTATCGGTC TGGGACCACG GTCCCACTTG TATTGTCGAT CAGACTATCA
  38341 GCGTGAGACT ACGATTCCAT CAATGCCTGT CAAGGGCAAG TATTGACATG TCGTCGTAAC
  38401 CTGTAGAACG GAGTAACCTC GGTGTGCGGT TGTATGCCTG CTGTGGATTG CTGCTGTGTC
  38461 CTGCTTATCC ACAACATTTT GCGCACGGTT ATGTGGAC
(Payload Vector 1)
SEQ ID NO: 9
          1 CCCCGAAAAG TGCCACCTGG GTCCTTTTCA TCACGTGCTA TAAAAATAAT TATAATTTAA
        61 ATTTTTTAAT ATAAATATAT AAATTAAAAA TAGAAAGTAA AAAAAGAAAT TAAAGAAAAA
      121 ATAGTTTTTG TTTTCCGAAG ATGTAAAAGA CTCTAGGGGG ATCGCCAACA AATACTACCT
      181 TTTATCTTGC TCTTCCTGCT CTCAGGTATT AATGCCGAAT TGTTTCATCT TGTCTGTGTA
      241 GAAGACCACA CACGAAAATC CTGTGATTTT ACATTTTACT TATCGTTAAT CGAATGTATA
      301 TCTATTTAAT CTGCTTTTCT TGTCTAATAA ATATATATGT AAAGTACGCT TTTTGTTGAA
      361 ATTTTTTAAA CCTTTGTTTA TTTTTTTTTC TTCATTCCGT AACTCTTCTA CCTTCTTTAT
      421 TTACTTTCTA AAATCCAAAT ACAAAACATA AAAATAAATA AACACAGAGT AAATTCCCAA
      481 ATTATTCCAT CATTAAAAGA TACGAGGCGC GTGTAAGTTA CAGGCAAGCG ATCCGTCCTA
      541 AGAAACCATT ATTATCATGA CATTAACCTA TAAAAATAGG CGTATCACGA GGCCCTTTCG
      601 TCTCGCGCGT TTCGGTGATG ACGGTGAAAA CCTCTGACAC ATGCAGCTCC CGGAGACGGT
      661 CACAGOTTGT CTGTAAGCGG ATGCCGGGAG CAGACAAGCC CGTCAGGGCG CGTCAGCGCG
      721 TGTTGGCGGG TGTCGGGGCT GGCTTAACTA TGCGGCATCA GAGCAGATTG TACTGAGAGT
      781 GCACCATAAA TTCCCGTTTT AAGAGCTTGG TGAGCGCTAG GAGTCACTGC CAGGTATCGT
      841 TTGAACACGG CATTAGTCAG GGAAGTCATA ACACAGTCCT TTCCCGCAAT TTTCTTTTTC
      901 TATTACTCTT GGCCTCCTCT AGTACACTCT ATATTTTTTT ATGCCTCGGT AATGATTTTC
      961 ATTTTTTTTT TTCCCCTAGC GGATGACTCT TTTTTTTTCT TAGCGATTGG CATTATCACA
    1021 TAATGAATTA TACATTATAT AAAGTAATGT GATTTCTTCG AAGAATATAC TAAAAAATGA
    1081 GCAGGCAAGA TAAACGAAGG CAAAGATGAC AGAGCAGAAA GCCCTAGTAA AGCGTATTAC
    1141 AAATGAAACC AAGATTCAGA TTGCGATCTC TTTAAAGGGT GGTCCCCTAG CGATAGAGCA
    1201 CTCGATCTTC CCAGAAAAAG AGGCAGAAGC AGTAGCAGAA CAGGCCACAC AATCGCAAGT
    1261 GATTAACGTC CACACAGGTA TAGGGTTTCT GGACCATATG ATACATGCTC TGGCCAAGCA
    1321 TTCCGGCTGG TCGCTAATCG TTGAGTGCAT TGGTGACTTA CACATAGACG ACCATCACAC
    1381 CACTGAAGAC TGCGGGATTG CTCTCGGTCA AGCTTTTAAA GAGGCCCTAC TGGCGCGTGG
    1441 AGTAAAAAGG TTTGGATCAG GATTTGCGCC TTTGGATGAG GCACTTTCCA GAGCGGTGGT
    1501 AGATCTTTCG AACAGGCCGT ACGCAGTTGT CGAACTTGGT TTGCAAAGGG AGAAAGTAGG
    1561 AGATCTCTCT TGCGAGATGA TCCCGCATTT TCTTGAAAGC TTTGCAGAGG CTAGCAGAAT
    1621 TACCCTCCAC GTTGATTGTC TGCGAGGCAA GAATGATCAT CACCGTAGTG AGAGTGCGTT
    1681 CAAGGCTCTT GCGGTTGCCA TAAGAGAAGC CACCTCGCCC AATGGTACCA ACGATGTTCC
    1741 CTCCACCAAA GGTGTTCTTA TGTAGTGACA CCGATTATTT AAAGCTGCAG CATACGATAT
    1801 ATATACATGT GTATATATGT ATACCTATGA ATGTCAGTAA GTATGTATAC GAACAGTATG
    1861 ATACTGAAGA TGACAAGGTA ATGCATCATT CTATACGTGT CATTCTGAAC GAGGCGCGCT
    1921 TTCCTTTTTT CTTTTTGCTT TTTCTTTTTT TTTCTCTTGA ACTCGACGGA TCTATGCGGT
    1981 GTGAAATACC GCACAGATGC GTCCTAGGGC GGCCGCATAA CTTCGTATAG CATACATTAT
    2041 ACGAAGTTAT CCGGAACCGA GTACAAGCCC ACGGTGCGCC TCGCCACCCG CGACGACGTC
    2101 CCCAGGGCCG TCCGCACCCT CGCCGCCGCG TTCGCCGACT ACCCCGCCAC GCGCCACACC
    2161 GTCGATCCGG ACCGCCACAT CGAGCGCGTG ACCGAGCTGC AAGAACTCTT CCTCACGCGC
    2221 GTCGGGCTCG ACATCGGCAA GGTGTGGGTC GCGGACGACG GAGCCGCTGT GGCGGTCTGG
    2281 ACCACGCCGG AGAGCGTCGA AGCGGGGGCG GTGTTCGCCG AGATCGGCCC GCGCATGGCC
    2341 GAGTTGAGCG GTTCCCGGCT GGCCGCGCAG CAACAGATGG AGGGCCTCCT GGCTCCGCAC
    2401 CGGCCCAAGG AGCCCGCGTG GTTCCTGGCC ACCGTCGGAG TCTCGCCCGA CCACCAGGGC
    2461 AAGGGTCTGG GCAGCGCCGT CGTGCTCCCC GGAGTGGAGG CGGCCGAGCG CGCCGGGGTG
    2521 CCCGCCTTCC TGGAAACCTC CGCGCCCCGC AACCTCCCCT TCTACGAGCG GCTCGGCTTC
    2581 ACCGTCACCG CCGACGTCGA GGTGCCCGAA GGACCGCGCA CCTGGTGCAT GACCCGCAAG
    2641 CCCGGTGCCG CATGCGGCAG TGGAGAGGGC AGAGGAAGTC TGCTAACATG CGGTGACGTC
    2701 GAGGAGAATC CTGGCCCAGG CGCCGTGAGC AAGGGCGAGG AGGATAACAT GGCCTCTCTC
    2761 CCAGCGACAC ATGAGTTACA CATCTTTGGC TCCATCAACG GTGTGGACTT TGACATGGTG
    2821 GGTCAGGGCA CCGGCAATCC AAATGATGGT TATGAGGAGT TAAACCTGAA GTCCACCAAG
    2881 GGCGACCTCC AGTTCTCCCC CTGGATTCTG GTCCCTCATA TCGGGTATGG CTTCCATCAG
    2941 TACCTGCCCT ACCCTGACGG GATGTCGCCT TTCCAGGCCG CCATGGTAGA TGGCTCTGGA
    3001 TACCAAGTCC ATCGCACAAT GCAGTTTGAA GATGGTGCCT CCCTTACTGT GAACTACCGC
    3061 TACACCTACG AGGGAAGCCA CATCAAAGGA GAGGCCCAGG TGAAGGGGAC TGGTTTCCCT
    3121 GCTGACGGTC CTGTGATGAC CAACTCGCTG ACCGCTGCGG ACTGGTGCAG GTCGAAGAAG
    3181 ACTTACCCCA ACGACAAAAC CATCATCAGT ACCTTTAAGT GGAGTTACAC CACTGGAAAT
    3241 GGCAAGCGCT ACCGGAGCAC TGCGCGGACC ACCTACACCT TTGCCAAGCC AATGGCGGCT
    3301 AACTATCTGA AGAACCAGCC GATGTACGTG TTCCGTAAGA CGGAACTCAA GCACTCCAAG
    3361 ACCGAGCTGA ACTTCAAGGA GTGGCAAAAG GCGTTTACCG ATGTGATGGG CATGGACGAG
    3421 CTGTACAAGT GAATTCGATC ATAATCAGCC ATACCACATT TGTAGAGGTT TTACTTGCTT
    3481 TAAAAAACCT CCCACACCTC CCCCTGAACC TGAAACATAA AATGAATGCA ATTGTTGTTG
    3541 TTAACTTGTT TATTGCAGCT TATAATGGTT ACAAATAAAG CAATAGCATC ACAAATTTCA
    3601 CAAATAAAGC ATTTTTTTCA CTGCATTCTA GTTGTGGTTT GTCCAAACTC ATCAATGTAT
    3661 CTTATCATGT CTGGGAGCTC ATAACTTCGT ATAGTATAGT ATATACGAAT TATACGCGTA
    3721 TCCATACTGG CGCGCCACAA CTTCGTATAG AAAGGTATAT ACGAAGTTGT GTCGACCAAT
    3781 TCTCATGTTT GACAGOTTAT CATCGAATTT CTGCCATTCA TCCGCTTATT ATCACTTATT
    3841 CAGGCGTAGC AACCAGGCGT TTAAGGGCAC CAATAACTGC CTTAAAAAAA TTAGAAAAAC
    3901 TCATCGAGCA TCAAATGAAA CTGCAATTTA TTCATATCAG GATTATCAAT ACCATATTTT
    3961 TGAAAAAGCC GTTTCTGTAA TGAAGGAGAA AACTCACCGA GGCAGTTCCA TAGGATGGCA
    4021 AGATCCTGGT ATCGGTCTGC GATTCCGACT CGTCCAACAT CAATACAACC TATTAATTTC
    4081 CCCTCGTCAA AAATAAGGTT ATCAAGTGAG AAATCACCAT GAGTGACGAC TGAATCCGGT
    4141 GAGAATGGCA AAAGCTTATG CATTTCTTTC CAGACTTGTT CAACAGGCCA GCCATTACGC
    4201 TCGTCATCAA AATCACTCGC ATCAACCAAA CCGTTATTCA TTCGTGATTG CGCCTGAGCG
    4261 AGACGAAATA CGCGATCGCT GTTAAAAGGA CAATTACAAA CAGGAATCGA ATGCAACCGG
    4321 CGCAGGAACA CTGCCAGCGC ATCAACAATA TTTTCACCTG AATCAGGATA TTCTTCTAAT
    4381 ACCTGGAATG CTGTTTTCCC TGGGATCGCA GTGGTGAGTA ACCATGCATC ATCAGGAGTA
    4441 CGGATAAAAT GCTTGATGGT CGGAAGAGGC ATAAATTCCG TCAGCCAGTT TAGTCTGACC
    4501 ATCTCATCTG TAACATCATT GGCAACGCTA CCTTTGCCAT GTTTCAGAAA CAACTCTGGC
    4561 GCATCGGGCT TCCCATACAA TCGATAGATT GTCGCACCTG ATTGCCCGAC ATTATCGCGA
    4621 GCCCATTTAT ACCCATATAA ATCAGCATCC ATGTTGGAAT TTAATCGCGG CCTCGAGCAA
    4681 GACGTTTCCC GTTGAATATG GCTCATTTTA GCTTCCTTAG CTCCTGAAAA TCTCGATAAC
    4741 TCAAAAAATA CGCCCGGTAG TGATCTTATT TCATTATGGT GAAAGTTGGA ACCTCTTACG
    4801 TGCCGATCAA CGTCTCATTT TCGCCAAAAG TTGGCCCAGG GCTTCCCGGT ATCAACAGGG
    4861 ACACCAGGAT TTATTTATTC TGCGAAGTGA TCTTCCGTCA CAGGTATTTA TTCGCGATAA
    4921 GCTCATGGAG CGGCGTAACC GTCGCACAGG AAGGACAGAG AAAGCGCGGA TCTGGGAAGT
    4981 GACGGACAGA ACGGTCAGGA CCTGGATTGG GGAGGCGGTT GCCGCCGCTG CTGCTGACGG
    5041 TGTGACGTTC TCTGTTCCGG TCACACCACA TACGTTCCGC CATTCCTATG CGATGCACAT
    5101 GCTGTATGCC GGTATACCGC TGAAAGTTCT GCAAAGCCTG ATGGGACATA AGTCCATCAG
    5161 TTCAACGGAA GTCTACACGA AGGTTTTTGC GCTGGATGTG GCTGCCCGGC ACCGGGTGCA
    5221 GTTTGCGATG CCGGAGTCTG ATGCGGTTGC GATGCTGAAA CAATTATCCT GAGAATAAAT
    5281 GCCTTGGCCT TTATATGGAA ATGTGGAACT GAGTGGATAT GCTGTTTTTG TCTGTTAAAC
    5341 AGAGAAGCTG GCTGTTATCC ACTGAGAAGC GAACGAAACA GTCGGGAAAA TCTCCCATTA
    5401 TCGTAGAGAT CCGCATTATT AATCTCAGGA GCCTGTGTAG CGTTTATAGG AAGTAGTGTT
    5461 CTGTCATGAT GCCTGCAAGC GGTAACGAAA ACGATTTGAA TATGCCTTCA GGAACAATAG
    5521 AAATCTTCGT GCGGTGTTAC GTTGAAGTGG AGCGGATTAT GTCAGCAATG GACAGAACAA
    5581 CCTAATGAAC ACAGAACCAT GATGTGGTCT GTCCTTTTAC AGCCAGTAGT GCTCGCCGCA
    5641 GTCGAGCGAC AGGGCGAAGC CCTCGAGCTG GTTGCCCTCG CCGCTGGGCT GGCGGCCGTC
    5701 TATGGCCCTG CAAACGCGCC AGAAACGCCG TCGAAGCCGT GTGCGAGACA CCGCGGCCGG
    5761 CCGCCGGCGT TGTGGATACC TCGCGGAAAA CTTGGCCCTC ACTGACAGAT GAGGGGCGGA
    5821 CGTTGACACT TGAGGGGCCG ACTCACCCGG CGCGGCGTTG ACAGATGAGG GGCAGGCTCG
    5881 ATTTCGGCCG GCGACGTGGA GCTGGCCAGC CTCGCAAATC GGCGAAAACG CCTGATTTTA
    5941 CGCGAGTTTC CCACAGATGA TGTGGACAAG CCTGGGGATA AGTGCCCTGC GGTATTGACA
    6001 CTTGAGGGGC GCGACTACTG ACAGATGAGG GGCGCGATCC TTGACACTTG AGGGGCAGAG
    6061 TGCTGACAGA TGAGGGGCGC ACCTATTGAC ATTTGAGGGG CTGTCCACAG GCAGAAAATC
    6121 CAGCATTTGC AAGGGTTTCC GCCCGTTTTT CGGCCACCGC TAACCTGTCT TTTAACCTGC
    6181 TTTTAAACCA ATATTTATAA ACCTTGTTTT TAACCAGGGC TGCGCCCTGT GCGCGTGACC
    6241 GCGCACGCCG AAGGGGGGTG CCCCCCCTTC TCGAACCCTC CCGGTCGAGT GAGCGAGGAA
    6301 GCACCAGGGA ACAGCACTTA TATATTCTGC TTACACACGA TGCCTGAAAA AACTTCCCTT
    6361 GGGGTTATCC ACTTATCCAC GGGGATATTT TTATAATTAT TTTTTTTATA GTTTTTAGAT
    6421 CTTCTTTTTT AGAGCGCCTT GTAGGCCTTT ATCCATGCTG GTTCTAGAGA AGGTGTTGTG
    6481 ACAAATTGCC CTTTCAGTGT GACAAATCAC CCTCAAATGA CAGTCCTGTC TGTGACAAAT
    6541 TGCCCTTAAC CCTGTGACAA ATTGCCCTCA GAAGAAGCTG TTTTTTCACA AAGTTATCCC
    6601 TGCTTATTGA CTCTTTTTTA TTTAGTGTGA CAATCTAAAA ACTTGTCACA CTTCACATGG
    6661 ATCTGTCATG GCGGAAACAG CGGTTATCAA TCACAAGAAA CGTAAAAATA GCCCGCGAAT
    6721 CGTCCAGTCA AACGACCTCA CTGAGGCGGC ATATAGTCTC TCCCGGGATC AAAAACGTAT
    6781 GCTGTATCTG TTCGTTGACC AGATCAGAAA ATCTGATGGC ACCCTACAGG AACATGACGG
    6841 TATCTGCGAG ATCCATGTTG CTAAATATGC TGAAATATTC GGATTGACCT CTGCGGAAGC
    6901 CAGTAAGGAT ATACGGCAGG CATTGAAGAG TTTCGCGGGG AAGGAAGTGG TTTTTTATCG
    6961 CCCTGAAGAG GATGCCGGCG ATGAAAAAGG CTATGAATCT TTTCCTTGGT TTATCAAACG
    7021 TGCGCACAGT CCATCCAGAG GGCTTTACAG TGTACATATC AACCCATATC TCATTCCCTT
    7081 CTTTATCGGG TTACAGAACC GGTTTACGCA GTTTCGGCTT AGTGAAACAA AAGAAATCAC
    7141 CAATCCGTAT GCCATGCGTT TATACGAATC CCTGTGTCAG TATCGTAAGC CGGATGGCTC
    7201 AGGCATCGTC TCTCTGAAAA TCGACTGGAT CATAGAGCGT TACCAGCTGC CTCAAAGTTA
    7261 CCAGCGTATG CCTGACTTCC GCCGCCGCTT CCTGCAGGTC TGTGTTAATG AGATCAACAG
    7321 CAGAACTCCA ATGCGCCTCT CATACATTGA GAAAAAGAAA GGCCGCCAGA CGACTCATAT
    7381 CGTATTTTCC TTCCGCGATA TCACTTCCAT GACGACAGGA TAGTCTGAGG GTTATCTGTC
    7441 ACAGATTTGA GGGTGGTTCG TCACATTTGT TCTGACCTAC TGAGGGTAAT TTGTCACAGT
    7501 TTTGCTGTTT CCTTCAGCCT GCATGGATTT TCTCATACTT TTTGAACTGT AATTTTTAAG
    7561 GAAGCCAAAT TTGAGGGCAG TTTGTCACAG TTGATTTCCT TCTCTTTCCC TTCGTCATGT
    7621 GACCTGATAT CGGGGGTTAG TTCGTCATCA TTGATGAGGG TTGATTATCA CAGTTTATTA
    7681 CTCTGAATTG GCTATCCGCG TGTGTACCTC TACCTGGAGT TTTTCCCACG GTGGATATTT
    7741 CTTCTTGCGC TGAGCGTAAG AGCTATCTGA CAGAACAGTT CTTCTTTGCT TCCTCGCCAG
    7801 TTCGCTCGCT ATGCTCGGTT ACACGGCTGC GGCGAGCGCT AGTGATAATA AGTGACTGAG
    7861 GTATGTGCTC TTCTTATCTC CTTTTGTAGT GTTGCTCTTA TTTTAAACAA CTTTGCGGTT
    7921 TTTTGATGAC TTTGCGATTT TGTTGTTGCT TTGCAGTAAA TTGCAAGATT TAATAAAAAA
    7981 ACGCAAAGCA ATGATTAAAG GATGTTCAGA ATGAAACTCA TGGAAACACT TAACCAGTGC
    8041 ATAAACGCTG GTCATGAAAT GACGAAGGCT ATCGCCATTG CACAGTTTAA TGATGACAGC
    8101 CCGGAAGCGA GGAAAATAAC CCGGCGCTGG AGAATAGGTG AAGCAGCGGA TTTAGTTGGG
    8161 GTTTCTTCTC AGGCTATCAG AGATGCCGAG AAAGCAGGGC GACTACCGCA CCCGGATATG
    8221 GAAATTCGAG GACGGGTTGA GCAACGTGTT GGTTATACAA TTGAACAAAT TAATCATATG
    8281 CGTGATGTGT TTGGTACGCG ATTGCGACGT GCTGAAGACG TATTTCCACC GGTGATCGGG
    8341 GTTGCTGCCC ATAAAGGTGG CGTTTACAAA ACCTCAGTTT CTGTTCATCT TGCTCAGGAT
    8401 CTGGCTCTGA AGGGGCTACG TGTTTTGCTC GTGGAAGGTA ACGACCCCCA GGGAACAGCC
    8461 TCAATGTATC ACGGATGGGT ACCAGATOTT CATATTCATG CAGAAGACAC TCTCCTGCCT
    8521 TTCTATCTTG GGGAAAAGGA CGATGTCACT TATGCAATAA AGCCCACTTG CTGGCCGGGG
    8581 CTTGACATTA TTCCTTCCTG TCTGGCTCTG CACCGTATTG AAACTGAGTT AATGGGCAAA
    8641 TTTGATGAAG GTAAACTGCC CACCGATCCA CACCTGATGC TCCGACTGGC CATTGAAACT
    8701 GTTGCTCATG ACTATGATGT CATAGTTATT GACAGCGCGC CTAACCTGGG TATCGGCACG
    8761 ATTAATGTCG TATGTGCTGC TGATGTGCTG ATTGTTCCCA CGCCTGCTGA GTTGTTTGAC
    8821 TACACCTCCG CACTGCAGTT TTTCGATATG CTTCGTGATC TGCTCAAGAA CGTTGATCTT
    8881 AAAGGGTTCG AGCCTGATGT ACGTATTTTG CTTACCAAAT ACAGCAATAG CAATGGCTCT
    8941 CAGTCCCCGT GGATGGAGGA GCAAATTCGG GATGCCTGGG GAAGCATGGT TCTAAAAAAT
    9001 GTTGTACGTG AAACGGATGA AGTTGGTAAA GGTCAGATCC GGATGAGAAC TGTTTTTGAA
    9061 CAGGCCATTG ATCAACGCTC TTCAACTGGT GCCTGGAGAA ATGCTCTTTC TATTTGGGAA
    9121 CCTGTCTGCA ATGAAATTTT CGATCGTCTG ATTAAACCAC GCTGGGAGAT TAGATAATGA
    9181 AGCGTGCGCC TGTTATTCCA AAACATACGC TCAATACTCA ACCGGTTGAA GATACTTCGT
    9241 TATCGACACC AGCTGCCCCG ATGGTGGATT CGTTAATTGC GCGCGTAGGA GTAATGGCTC
    9301 GCGGTAATGC CATTACTTTG CCTGTATGTG GTCGGGATGT GAAGTTTACT CTTGAAGTGC
    9361 TCCGGGGTGA TAGTGTTGAG AAGACCTCTC GGGTATGGTC AGGTAATGAA CGTGACCAGG
    9421 AGCTGCTTAC TGAGGACGCA CTGGATGATC TCATCCCTTC TTTTCTACTG ACTGGTCAAC
    9481 AGACACCGGC GTTCGGTCGA AGAGTATCTG GTGTCATAGA AATTGCCGAT GGGAGTCGCC
    9541 GTCGTAAAGC TGCTGCACTT ACCGAAAGTG ATTATCGTGT TCTGGTTGGC GAGCTGGATG
    9601 ATGAGCAGAT GGCTGCATTA TCCAGATTGG GTAACGATTA TCGCCCAACA AGTGCTTATG
    9661 AACGTGGTCA GCGTTATGCA AGCCGATTGC AGAATGAATT TGCTGGAAAT ATTTCTGCGC
    9721 TGGCTGATGC GGAAAATATT TCACGTAAGA TTATTACCCG CTGTATCAAC ACCGCCAAAT
    9781 TGCCTAAATC AGTTGTTGCT CTTTTTTCTC ACCCCGGTGA ACTATCTGCC CGGTCAGGTG
    9841 ATGCACTTCA AAAAGCCTTT ACAGATAAAG AGGAATTACT TAAGCAGCAG GCATCTAACC
    9901 TTCATGAGCA GAAAAAAGCT GGGGTGATAT TTGAAGCTGA AGAAGTTATC ACTCTTTTAA
    9961 CTTCTGTGCT TAAAACGTCA TCTGCATCAA GAACTAGTTT AAGCTCACGA CATCAGTTTG
  10021 CTCCTGGAGC GACAGTATTG TATAAGGGCG ATAAAATGGT GCTTAACCTG GACAGGTCTC
  10081 GTGTTCCAAC TGAGTGTATA GAGAAAATTG AGGCCATTCT TAAGGAACTT GAAAAGCCAG
  10141 CACCCTGATG CGACCACGTT TTAGTCTACG TTTATCTGTC TTTACTTAAT GTCCTTTGTT
  10201 ACAGGCCAGA AAGCATAACT GGCCTGAATA TTCTCTCTGG GCCCACTGTT CCACTTGTAT
  10261 CGTCGGTCTG ATAATCAGAC TGGGACCACG GTCCCACTCG TATCGTCGGT CTGATTATTA
  10321 GTCTGGGACC ACGGTCCCAC TCGTATCGTC GGTCTGATTA TTAGTCTGGG ACCACGGTCC
  10381 CACTCGTATC GTCGGTCTGA TAATCAGACT GGGACCACGG TCCCACTCGT ATCGTCGGTC
  10441 TGATTATTAG TCTGGGACCA TGGTCCCACT CGTATCGTCG GTCTGATTAT TAGTCTGGGA
  10501 CCACGGTCCC ACTCGTATCG TCGGTCTGAT TATTAGTCTG GAACCACGGT CCCACTCGTA
  10561 TCGTCGGTCT GATTATTAGT CTGGGACCAC GGTCCCACTC GTATCGTCGG TCTGATTATT
  10621 AGTCTGGGAC CACGATCCCA CTCGTGTTGT CGGTCTGATT ATCGGTCTGG GACCACGGTC
  10681 CCACTTGTAT TGTCGATCAG ACTATCAGCG TGAGACTACG ATTCCATCAA TGCCTGTCAA
  10741 GGGCAAGTAT TGACATGTCG TCGTAACCTG TAGAACGGAG TAACCTCGGT GTGCGGTTGT
  10801 ATGCCTGCTG TGGATTGCTG CTGTGTCCTG CTTATCCACA ACATTTTGCG CACGGTTATG
  10861 TGGAC
(Payload Vector 2)
SEQ ID NO: 10
          1 CCCCGAAAAG TGCCACCTGG GTCCTTTTCA TCACGTGCTA TAAAAATAAT TATAATTTAA
        61 ATTTTTTAAT ATAAATATAT AAATTAAAAA TAGAAAGTAA AAAAAGAAAT TAAAGAAAAA
      121 ATAGTTTTTG TTTTCCGAAG ATGTAAAAGA CTCTAGGGGG ATCGCCAACA AATACTACCT
      181 TTTATCTTGC TCTTCCTGCT CTCAGGTATT AATGCCGAAT TGTTTCATCT TGTCTGTGTA
      241 GAAGACCACA CACGAAAATC CTGTGATTTT ACATTTTACT TATCGTTAAT CGAATGTATA
      301 TCTATTTAAT CTGCTTTTCT TGTCTAATAA ATATATATGT AAAGTACGCT TTTTGTTGAA
      361 ATTTTTTAAA CCTTTGTTTA TTTTTTTTTC TTCATTCCGT AACTCTTCTA CCTTCTTTAT
      421 TTACTTTCTA AAATCCAAAT ACAAAACATA AAAATAAATA AACACAGAGT AAATTCCCAA
      481 ATTATTCCAT CATTAAAAGA TACGAGGCGC GTGTAAGTTA CAGGCAAGCG ATCCGTCCTA
      541 AGAAACCATT ATTATCATGA CATTAACCTA TAAAAATAGG CGTATCACGA GGCCCTTTCG
      601 TCTCGCGCGT TTCGGTGATG ACGGTGAAAA CCTCTGACAC ATGCAGCTCC CGGAGACGGT
      661 CACAGOTTGT CTGTAAGCGG ATGCCGGGAG CAGACAAGCC CGTCAGGGCG CGTCAGCGCG
      721 TGTTGGCGGG TGTCGGGGCT GGCTTAACTA TGCGGCATCA GAGCAGATTG TACTGAGAGT
      781 GCACCATAAA TTCCCGTTTT AAGAGCTTGG TGAGCGCTAG GAGTCACTGC CAGGTATCGT
      841 TTGAACACGG CATTAGTCAG GGAAGTCATA ACACAGTCCT TTCCCGCAAT TTTCTTTTTC
      901 TATTACTCTT GGCCTCCTCT AGTACACTCT ATATTTTTTT ATGCCTCGGT AATGATTTTC
      961 ATTTTTTTTT TTCCCCTAGC GGATGACTCT TTTTTTTTCT TAGCGATTGG CATTATCACA
    1021 TAATGAATTA TACATTATAT AAAGTAATGT GATTTCTTCG AAGAATATAC TAAAAAATGA
    1081 GCAGGCAAGA TAAACGAAGG CAAAGATGAC AGAGCAGAAA GCCCTAGTAA AGCGTATTAC
    1141 AAATGAAACC AAGATTCAGA TTGCGATCTC TTTAAAGGGT GGTCCCCTAG CGATAGAGCA
    1201 CTCGATCTTC CCAGAAAAAG AGGCAGAAGC AGTAGCAGAA CAGGCCACAC AATCGCAAGT
    1261 GATTAACGTC CACACAGGTA TAGGGTTTCT GGACCATATG ATACATGCTC TGGCCAAGCA
    1321 TTCCGGCTGG TCGCTAATCG TTGAGTGCAT TGGTGACTTA CACATAGACG ACCATCACAC
    1381 CACTGAAGAC TGCGGGATTG CTCTCGGTCA AGCTTTTAAA GAGGCCCTAC TGGCGCGTGG
    1441 AGTAAAAAGG TTTGGATCAG GATTTGCGCC TTTGGATGAG GCACTTTCCA GAGCGGTGGT
    1501 AGATCTTTCG AACAGGCCGT ACGCAGTTGT CGAACTTGGT TTGCAAAGGG AGAAAGTAGG
    1561 AGATCTCTCT TGCGAGATGA TCCCGCATTT TCTTGAAAGC TTTGCAGAGG CTAGCAGAAT
    1621 TACCCTCCAC GTTGATTGTC TGCGAGGCAA GAATGATCAT CACCGTAGTG AGAGTGCGTT
    1681 CAAGGCTCTT GCGGTTGCCA TAAGAGAAGC CACCTCGCCC AATGGTACCA ACGATGTTCC
    1741 CTCCACCAAA GGTGTTCTTA TGTAGTGACA CCGATTATTT AAAGCTGCAG CATACGATAT
    1801 ATATACATGT GTATATATGT ATACCTATGA ATGTCAGTAA GTATGTATAC GAACAGTATG
    1861 ATACTGAAGA TGACAAGGTA ATGCATCATT CTATACGTGT CATTCTGAAC GAGGCGCGCT
    1921 TTCCTTTTTT CTTTTTGCTT TTTCTTTTTT TTTCTCTTGA ACTCGACGGA TCTATGCGGT
    1981 GTGAAATACC GCACAGATGC GTCCTAGGGC GGCCGCATAA CTTCGTATAG CATACATTAT
    2041 ACGAAGTTAT CCGGAAAAAC ATTTAACATT TCTCAACAAG ATCTAGAATT AGTAGAAGTA
    2101 GCGACAGAGA AGATTACAAT GCTTTATGAG GATAATAAAC ATCATGTGGG AGCGGCAATT
    2161 CGTACGAAAA CAGGAGAAAT CATTTCGGCA GTACATATTG AAGCGTATAT AGGACGAGTA
    2221 ACTGTTTGTG CAGAAGCCAT TGCGATTGGT AGTGCAGTTT CGAATGGACA AAAGGATTTT
    2281 GACACGATTG TAGCTGTTAG ACACCCTTAT TCTGACGAAG TAGATAGAAG TATTCGAGTG
    2341 GTAAGTCCTT GTGGTATGTG TAGGGAGTTG ATTTCAGACT ATGCACCAGA TTGTTTTGTG
    2401 TTAATAGAAA TGAATGGCAA GTTAGTCAAA ACTACGATTG AAGAACTCAT TCCACTCAAA
    2461 TATACCCGAA ATGGCAGTGG AGAGGGCAGA GGAAGTCTGC TAACATGCGG TGACGTCGAG
    2521 GAGAATCCTG GCCCAGTGAG CAAGGGCGAG GCAGTGATCA AGGAGTTCAT GCGGTTCAAG
    2581 GTGCACATGG AGGGCTCCAT GAACGGCCAC GAGTTCGAGA TCGAGGGCGA GGGCGAGGGC
    2641 CGCCCCTACG AGGGCACCCA GACCGCCAAG CTGAAGGTGA CCAAGGGTGG CCCCCTGCCC
    2701 TTCTCCTGGG ACATCCTGTC CCCTCAGTTC ATGTACGGCT CCAGGGCCTT CACCAAGCAC
    2761 CCCGCCGACA TCCCCGACTA CTATAAGCAG TCCTTCCCCG AGGGCTTCAA GTGGGAGCGC
    2821 GTGATGAACT TCGAGGACGG CGGCGCCGTG ACCGTGACCC AGGACACCTC CCTGGAGGAC
    2881 GGCACCCTGA TCTACAAGGT GAAGCTCCGC GGCACCAACT TCCCTCCTGA CGGCCCCGTA
    2941 ATGCAGAAGA AAACAATGGG CTGGGAAGCG TCCACCGAGC GGTTGTACCC CGAGGACGGC
    3001 GTGCTGAAGG GCGACATTAA GATGGCCCTG CGCCTGAAGG ACGGCGGCCG CTACCTGGCG
    3061 GACTTCAAGA CCACCTACAA GGCCAAGAAG CCCGTGCAGA TGCCCGGCGC CTACAACGTT
    3121 GACCGCAAGT TGGACATCAC CTCCCACAAC GAGGACTACA CCGTGGTGGA ACAGTACGAA
    3181 CGCTCCGAGG GCCGCCACTC CACCGGCGGC ATGGACGAGC TGTACAAGTG AATTCGATCA
    3241 TAATCAGCCA TACCACATTT GTAGAGGTTT TACTTGCTTT AAAAAACCTC CCACACCTCC
    3301 CCCTGAACCT GAAACATAAA ATGAATGCAA TTGTTGTTGT TAACTTGTTT ATTGCAGCTT
    3361 ATAATGGTTA CAAATAAAGC AATAGCATCA CAAATTTCAC AAATAAAGCA TTTTTTTCAC
    3421 TGCATTCTAG TTGTGGTTTG TCCAAACTCA TCAATGTATC TTATCATGTC TGGGAGCTCA
    3481 TAACTTCGTA TAGCGTGCGT TATACGAAGT TATACGCGTA TCCATACTGG CGCGCCATAA
    3541 CTTCGTATAG TATAGTATAT ACGAATTATG TCGACCAATT CTCATGTTTG ACAGCTTATC
    3601 ATCGAATTTC TGCCATTCAT CCGCTTATTA TCACTTATTC AGGCGTAGCA ACCAGGCGTT
    3661 TAAGGGCACC AATAACTGCC TTAAAAAAAT TAGAAAAACT CATCGAGCAT CAAATGAAAC
    3721 TGCAATTTAT TCATATCAGG ATTATCAATA CCATATTTTT GAAAAAGCCG TTTCTGTAAT
    3781 GAAGGAGAAA ACTCACCGAG GCAGTTCCAT AGGATGGCAA GATCCTGGTA TCGGTCTGCG
    3841 ATTCCGACTC GTCCAACATC AATACAACCT ATTAATTTCC CCTCGTCAAA AATAAGGTTA
    3901 TCAAGTGAGA AATCACCATG AGTGACGACT GAATCCGGTG AGAATGGCAA AAGCTTATGC
    3961 ATTTCTTTCC AGACTTGTTC AACAGGCCAG CCATTACGCT CGTCATCAAA ATCACTCGCA
    4021 TCAACCAAAC CGTTATTCAT TCGTGATTGC GCCTGAGCGA GACGAAATAC GCGATCGCTG
    4081 TTAAAAGGAC AATTACAAAC AGGAATCGAA TGCAACCGGC GCAGGAACAC TGCCAGCGCA
    4141 TCAACAATAT TTTCACCTGA ATCAGGATAT TCTTCTAATA CCTGGAATGC TGTTTTCCCT
    4201 GGGATCGCAG TGGTGAGTAA CCATGCATCA TCAGGAGTAC GGATAAAATG CTTGATGGTC
    4261 GGAAGAGGCA TAAATTCCGT CAGCCAGTTT AGTCTGACCA TCTCATCTGT AACATCATTG
    4321 GCAACGCTAC CTTTGCCATG TTTCAGAAAC AACTCTGGCG CATCGGGCTT CCCATACAAT
    4381 CGATAGATTG TCGCACCTGA TTGCCCGACA TTATCGCGAG CCCATTTATA CCCATATAAA
    4441 TCAGCATCCA TGTTGGAATT TAATCGCGGC CTCGAGCAAG ACGTTTCCCG TTGAATATGG
    4501 CTCATTTTAG CTTCCTTAGC TCCTGAAAAT CTCGATAACT CAAAAAATAC GCCCGGTAGT
    4561 GATCTTATTT CATTATGGTG AAAGTTGGAA CCTCTTACGT GCCGATCAAC GTCTCATTTT
    4621 CGCCAAAAGT TGGCCCAGGG CTTCCCGGTA TCAACAGGGA CACCAGGATT TATTTATTCT
    4681 GCGAAGTGAT CTTCCGTCAC AGGTATTTAT TCGCGATAAG CTCATGGAGC GGCGTAACCG
    4741 TCGCACAGGA AGGACAGAGA AAGCGCGGAT CTGGGAAGTG ACGGACAGAA CGGTCAGGAC
    4801 CTGGATTGGG GAGGCGGTTG CCGCCGCTGC TGCTGACGGT GTGACGTTCT CTGTTCCGGT
    4861 CACACCACAT ACGTTCCGCC ATTCCTATGC GATGCACATG CTGTATGCCG GTATACCGCT
    4921 GAAAGTTCTG CAAAGCCTGA TGGGACATAA GTCCATCAGT TCAACGGAAG TCTACACGAA
    4981 GGTTTTTGCG CTGGATGTGG CTGCCCGGCA CCGGGTGCAG TTTGCGATGC CGGAGTCTGA
    5041 TGCGGTTGCG ATGCTGAAAC AATTATCCTG AGAATAAATG CCTTGGCCTT TATATGGAAA
    5101 TGTGGAACTG AGTGGATATG CTGTTTTTGT CTGTTAAACA GAGAAGCTGG CTGTTATCCA
    5161 CTGAGAAGCG AACGAAACAG TCGGGAAAAT CTCCCATTAT CGTAGAGATC CGCATTATTA
    5221 ATCTCAGGAG CCTGTGTAGC GTTTATAGGA AGTAGTGTTC TGTCATGATG CCTGCAAGCG
    5281 GTAACGAAAA CGATTTGAAT ATGCCTTCAG GAACAATAGA AATCTTCGTG CGGTGTTACG
    5341 TTGAAGTGGA GCGGATTATG TCAGCAATGG ACAGAACAAC CTAATGAACA CAGAACCATG
    5401 ATGTGGTCTG TCCTTTTACA GCCAGTAGTG CTCGCCGCAG TCGAGCGACA GGGCGAAGCC
    5461 CTCGAGCTGG TTGCCCTCGC CGCTGGGCTG GCGGCCGTCT ATGGCCCTGC AAACGCGCCA
    5521 GAAACGCCGT CGAAGCCGTG TGCGAGACAC CGCGGCCGGC CGCCGGCGTT GTGGATACCT
    5581 CGCGGAAAAC TTGGCCCTCA CTGACAGATG AGGGGCGGAC GTTGACACTT GAGGGGCCGA
    5641 CTCACCCGGC GCGGCGTTGA CAGATGAGGG GCAGGCTCGA TTTCGGCCGG CGACGTGGAG
    5701 CTGGCCAGCC TCGCAAATCG GCGAAAACGC CTGATTTTAC GCGAGTTTCC CACAGATGAT
    5761 GTGGACAAGC CTGGGGATAA GTGCCCTGCG GTATTGACAC TTGAGGGGCG CGACTACTGA
    5821 CAGATGAGGG GCGCGATCCT TGACACTTGA GGGGCAGAGT GCTGACAGAT GAGGGGCGCA
    5881 CCTATTGACA TTTGAGGGGC TGTCCACAGG CAGAAAATCC AGCATTTGCA AGGGTTTCCG
    5941 CCCGTTTTTC GGCCACCGCT AACCTGTCTT TTAACCTGCT TTTAAACCAA TATTTATAAA
    6001 CCTTGTTTTT AACCAGGGCT GCGCCCTGTG CGCGTGACCG CGCACGCCGA AGGGGGGTGC
    6061 CCCCCCTTCT CGAACCCTCC CGGTCGAGTG AGCGAGGAAG CACCAGGGAA CAGCACTTAT
    6121 ATATTCTGCT TACACACGAT GCCTGAAAAA ACTTCCCTTG GGGTTATCCA CTTATCCACG
    6181 GGGATATTTT TATAATTATT TTTTTTATAG TTTTTAGATC TTCTTTTTTA GAGCGCCTTG
    6241 TAGGCCTTTA TCCATGCTGG TTCTAGAGAA GGTGTTGTGA CAAATTGCCC TTTCAGTGTG
    6301 ACAAATCACC CTCAAATGAC AGTCCTGTCT GTGACAAATT GCCCTTAACC CTGTGACAAA
    6361 TTGCCCTCAG AAGAAGCTGT TTTTTCACAA AGTTATCCCT GCTTATTGAC TCTTTTTTAT
    6421 TTAGTGTGAC AATCTAAAAA CTTGTCACAC TTCACATGGA TCTGTCATGG CGGAAACAGC
    6481 GGTTATCAAT CACAAGAAAC GTAAAAATAG CCCGCGAATC GTCCAGTCAA ACGACCTCAC
    6541 TGAGGCGGCA TATAGTCTCT CCCGGGATCA AAAACGTATG CTGTATCTGT TCGTTGACCA
    6601 GATCAGAAAA TCTGATGGCA CCCTACAGGA ACATGACGGT ATCTGCGAGA TCCATGTTGC
    6661 TAAATATGCT GAAATATTCG GATTGACCTC TGCGGAAGCC AGTAAGGATA TACGGCAGGC
    6721 ATTGAAGAGT TTCGCGGGGA AGGAAGTGGT TTTTTATCGC CCTGAAGAGG ATGCCGGCGA
    6781 TGAAAAAGGC TATGAATCTT TTCCTTGGTT TATCAAACGT GCGCACAGTC CATCCAGAGG
    6841 GCTTTACAGT GTACATATCA ACCCATATCT CATTCCCTTC TTTATCGGGT TACAGAACCG
    6901 GTTTACGCAG TTTCGGCTTA GTGAAACAAA AGAAATCACC AATCCGTATG CCATGCGTTT
    6961 ATACGAATCC CTGTGTCAGT ATCGTAAGCC GGATGGCTCA GGCATCGTCT CTCTGAAAAT
    7021 CGACTGGATC ATAGAGCGTT ACCAGCTGCC TCAAAGTTAC CAGCGTATGC CTGACTTCCG
    7081 CCGCCGCTTC CTGCAGGTCT GTGTTAATGA GATCAACAGC AGAACTCCAA TGCGCCTCTC
    7141 ATACATTGAG AAAAAGAAAG GCCGCCAGAC GACTCATATC GTATTTTCCT TCCGCGATAT
    7201 CACTTCCATG ACGACAGGAT AGTCTGAGGG TTATCTGTCA CAGATTTGAG GGTGGTTCGT
    7261 CACATTTGTT CTGACCTACT GAGGGTAATT TGTCACAGTT TTGCTGTTTC CTTCAGCCTG
    7321 CATGGATTTT CTCATACTTT TTGAACTGTA ATTTTTAAGG AAGCCAAATT TGAGGGCAGT
    7381 TTGTCACAGT TGATTTCCTT CTCTTTCCCT TCGTCATGTG ACCTGATATC GGGGGTTAGT
    7441 TCGTCATCAT TGATGAGGGT TGATTATCAC AGTTTATTAC TCTGAATTGG CTATCCGCGT
    7501 GTGTACCTCT ACCTGGAGTT TTTCCCACGG TGGATATTTC TTCTTGCGCT GAGCGTAAGA
    7561 GCTATCTGAC AGAACAGTTC TTCTTTGCTT CCTCGCCAGT TCGCTCGCTA TGCTCGGTTA
    7621 CACGGCTGCG GCGAGCGCTA GTGATAATAA GTGACTGAGG TATGTGCTCT TCTTATCTCC
    7681 TTTTGTAGTG TTGCTCTTAT TTTAAACAAC TTTGCGGTTT TTTGATGACT TTGCGATTTT
    7741 GTTGTTGCTT TGCAGTAAAT TGCAAGATTT AATAAAAAAA CGCAAAGCAA TGATTAAAGG
    7801 ATGTTCAGAA TGAAACTCAT GGAAACACTT AACCAGTGCA TAAACGCTGG TCATGAAATG
    7861 ACGAAGGCTA TCGCCATTGC ACAGTTTAAT GATGACAGCC CGGAAGCGAG GAAAATAACC
    7921 CGGCGCTGGA GAATAGGTGA AGCAGCGGAT TTAGTTGGGG TTTCTTCTCA GGCTATCAGA
    7981 GATGCCGAGA AAGCAGGGCG ACTACCGCAC CCGGATATGG AAATTCGAGG ACGGGTTGAG
    8041 CAACGTGTTG GTTATACAAT TGAACAAATT AATCATATGC GTGATGTGTT TGGTACGCGA
    8101 TTGCGACGTG CTGAAGACGT ATTTCCACCG GTGATCGGGG TTGCTGCCCA TAAAGGTGGC
    8161 GTTTACAAAA CCTCAGTTTC TGTTCATCTT GCTCAGGATC TGGCTCTGAA GGGGCTACGT
    8221 GTTTTGCTCG TGGAAGGTAA CGACCCCCAG GGAACAGCCT CAATGTATCA CGGATGGGTA
    8281 CCAGATCTTC ATATTCATGC AGAAGACACT CTCCTGCCTT TCTATCTTGG GGAAAAGGAC
    8341 GATGTCACTT ATGCAATAAA GCCCACTTGC TGGCCGGGGC TTGACATTAT TCCTTCCTGT
    8401 CTGGCTCTGC ACCGTATTGA AACTGAGTTA ATGGGCAAAT TTGATGAAGG TAAACTGCCC
    8461 ACCGATCCAC ACCTGATGCT CCGACTGGCC ATTGAAACTG TTGCTCATGA CTATGATGTC
    8521 ATAGTTATTG ACAGCGCGCC TAACCTGGGT ATCGGCACGA TTAATGTCGT ATGTGCTGCT
    8581 GATGTGCTGA TTGTTCCCAC GCCTGCTGAG TTGTTTGACT ACACCTCCGC ACTGCAGTTT
    8641 TTCGATATGC TTCGTGATCT GCTCAAGAAC GTTGATCTTA AAGGGTTCGA GCCTGATGTA
    8701 CGTATTTTGC TTACCAAATA CAGCAATAGC AATGGCTCTC AGTCCCCGTG GATGGAGGAG
    8761 CAAATTCGGG ATGCCTGGGG AAGCATGGTT CTAAAAAATG TTGTACGTGA AACGGATGAA
    8821 GTTGGTAAAG GTCAGATCCG GATGAGAACT GTTTTTGAAC AGGCCATTGA TCAACGCTCT
    8881 TCAACTGGTG CCTGGAGAAA TGCTCTTTCT ATTTGGGAAC CTGTCTGCAA TGAAATTTTC
    8941 GATCGTCTGA TTAAACCACG CTGGGAGATT AGATAATGAA GCGTGCGCCT GTTATTCCAA
    9001 AACATACGCT CAATACTCAA CCGGTTGAAG ATACTTCGTT ATCGACACCA GCTGCCCCGA
    9061 TGGTGGATTC GTTAATTGCG CGCGTAGGAG TAATGGCTCG CGGTAATGCC ATTACTTTGC
    9121 CTGTATGTGG TCGGGATGTG AAGTTTACTC TTGAAGTGCT CCGGGGTGAT AGTGTTGAGA
    9181 AGACCTCTCG GGTATGGTCA GGTAATGAAC GTGACCAGGA GCTGCTTACT GAGGACGCAC
    9241 TGGATGATCT CATCCCTTCT TTTCTACTGA CTGGTCAACA GACACCGGCG TTCGGTCGAA
    9301 GAGTATCTGG TGTCATAGAA ATTGCCGATG GGAGTCGCCG TCGTAAAGCT GCTGCACTTA
    9361 CCGAAAGTGA TTATCGTGTT CTGGTTGGCG AGCTGGATGA TGAGCAGATG GCTGCATTAT
    9421 CCAGATTGGG TAACGATTAT CGCCCAACAA GTGCTTATGA ACGTGGTCAG CGTTATGCAA
    9481 GCCGATTGCA GAATGAATTT GCTGGAAATA TTTCTGCGCT GGCTGATGCG GAAAATATTT
    9541 CACGTAAGAT TATTACCCGC TGTATCAACA CCGCCAAATT GCCTAAATCA GTTGTTGCTC
    9601 TTTTTTCTCA CCCCGGTGAA CTATCTGCCC GGTCAGGTGA TGCACTTCAA AAAGCCTTTA
    9661 CAGATAAAGA GGAATTACTT AAGCAGCAGG CATCTAACCT TCATGAGCAG AAAAAAGCTG
    9721 GGGTGATATT TGAAGCTGAA GAAGTTATCA CTCTTTTAAC TTCTGTGCTT AAAACGTCAT
    9781 CTGCATCAAG AACTAGTTTA AGCTCACGAC ATCAGTTTGC TCCTGGAGCG ACAGTATTGT
    9841 ATAAGGGCGA TAAAATGGTG CTTAACCTGG ACAGGTCTCG TGTTCCAACT GAGTGTATAG
    9901 AGAAAATTGA GGCCATTCTT AAGGAACTTG AAAAGCCAGC ACCCTGATGC GACCACGTTT
    9961 TAGTCTACGT TTATCTGTCT TTACTTAATG TCCTTTGTTA CAGGCCAGAA AGCATAACTG
  10021 GCCTGAATAT TCTCTCTGGG CCCACTGTTC CACTTGTATC GTCGGTCTGA TAATCAGACT
  10081 GGGACCACGG TCCCACTCGT ATCGTCGGTC TGATTATTAG TCTGGGACCA CGGTCCCACT
  10141 CGTATCGTCG GTCTGATTAT TAGTCTGGGA CCACGGTCCC ACTCGTATCG TCGGTCTGAT
  10201 AATCAGACTG GGACCACGGT CCCACTCGTA TCGTCGGTCT GATTATTAGT CTGGGACCAT
  10261 GGTCCCACTC GTATCGTCGG TCTGATTATT AGTCTGGGAC CACGGTCCCA CTCGTATCGT
  10321 CGGTCTGATT ATTAGTCTGG AACCACGGTC CCACTCGTAT CGTCGGTCTG ATTATTAGTC
  10381 TGGGACCACG GTCCCACTCG TATCGTCGGT CTGATTATTA GTCTGGGACC ACGATCCCAC
  10441 TCGTGTTGTC GGTCTGATTA TCGGTCTGGG ACCACGGTCC CACTTGTATT GTCGATCAGA
  10501 CTATCAGCGT GAGACTACGA TTCCATCAAT GCCTGTCAAG GGCAAGTATT GACATGTCGT
  10561 CGTAACCTGT AGAACGGAGT AACCTCGGTG TGCGGTTGTA TGCCTGCTGT GGATTGCTGC
  10621 TGTGTCCTGC TTATCCACAA CATTTTGCGC ACGGTTATGT GGAC
(Payload Vector Exit1_FlpOUTneo)
SEQ ID NO: 11
          1 CCCCGAAAAG TGCCACCTGG GTCCTTTTCA TCACGTGCTA TAAAAATAAT TATAATTTAA
        61 ATTTTTTAAT ATAAATATAT AAATTAAAAA TAGAAAGTAA AAAAAGAAAT TAAAGAAAAA
      121 ATAGTTTTTG TTTTCCGAAG ATGTAAAAGA CTCTAGGGGG ATCGCCAACA AATACTACCT
      181 TTTATCTTGC TCTTCCTGCT CTCAGGTATT AATGCCGAAT TGTTTCATCT TGTCTGTGTA
      241 GAAGACCACA CACGAAAATC CTGTGATTTT ACATTTTACT TATCGTTAAT CGAATGTATA
      301 TCTATTTAAT CTGCTTTTCT TGTCTAATAA ATATATATGT AAAGTACGCT TTTTGTTGAA
      361 ATTTTTTAAA CCTTTGTTTA TTTTTTTTTC TTCATTCCGT AACTCTTCTA CCTTCTTTAT
      421 TTACTTTCTA AAATCCAAAT ACAAAACATA AAAATAAATA AACACAGAGT AAATTCCCAA
      481 ATTATTCCAT CATTAAAAGA TACGAGGCGC GTGTAAGTTA CAGGCAAGCG ATCCGTCCTA
      541 AGAAACCATT ATTATCATGA CATTAACCTA TAAAAATAGG CGTATCACGA GGCCCTTTCG
      601 TCTCGCGCGT TTCGGTGATG ACGGTGAAAA CCTCTGACAC ATGCAGCTCC CGGAGACGGT
      661 CACAGOTTGT CTGTAAGCGG ATGCCGGGAG CAGACAAGCC CGTCAGGGCG CGTCAGCGCG
      721 TGTTGGCGGG TGTCGGGGCT GGCTTAACTA TGCGGCATCA GAGCAGATTG TACTGAGAGT
      781 GCACCATAAA TTCCCGTTTT AAGAGCTTGG TGAGCGCTAG GAGTCACTGC CAGGTATCGT
      841 TTGAACACGG CATTAGTCAG GGAAGTCATA ACACAGTCCT TTCCCGCAAT TTTCTTTTTC
      901 TATTACTCTT GGCCTCCTCT AGTACACTCT ATATTTTTTT ATGCCTCGGT AATGATTTTC
      961 ATTTTTTTTT TTCCCCTAGC GGATGACTCT TTTTTTTTCT TAGCGATTGG CATTATCACA
    1021 TAATGAATTA TACATTATAT AAAGTAATGT GATTTCTTCG AAGAATATAC TAAAAAATGA
    1081 GCAGGCAAGA TAAACGAAGG CAAAGATGAC AGAGCAGAAA GCCCTAGTAA AGCGTATTAC
    1141 AAATGAAACC AAGATTCAGA TTGCGATCTC TTTAAAGGGT GGTCCCCTAG CGATAGAGCA
    1201 CTCGATCTTC CCAGAAAAAG AGGCAGAAGC AGTAGCAGAA CAGGCCACAC AATCGCAAGT
    1261 GATTAACGTC CACACAGGTA TAGGGTTTCT GGACCATATG ATACATGCTC TGGCCAAGCA
    1321 TTCCGGCTGG TCGCTAATCG TTGAGTGCAT TGGTGACTTA CACATAGACG ACCATCACAC
    1381 CACTGAAGAC TGCGGGATTG CTCTCGGTCA AGCTTTTAAA GAGGCCCTAC TGGCGCGTGG
    1441 AGTAAAAAGG TTTGGATCAG GATTTGCGCC TTTGGATGAG GCACTTTCCA GAGCGGTGGT
    1501 AGATCTTTCG AACAGGCCGT ACGCAGTTGT CGAACTTGGT TTGCAAAGGG AGAAAGTAGG
    1561 AGATCTCTCT TGCGAGATGA TCCCGCATTT TCTTGAAAGC TTTGCAGAGG CTAGCAGAAT
    1621 TACCCTCCAC GTTGATTGTC TGCGAGGCAA GAATGATCAT CACCGTAGTG AGAGTGCGTT
    1681 CAAGGCTCTT GCGGTTGCCA TAAGAGAAGC CACCTCGCCC AATGGTACCA ACGATGTTCC
    1741 CTCCACCAAA GGTGTTCTTA TGTAGTGACA CCGATTATTT AAAGCTGCAG CATACGATAT
    1801 ATATACATGT GTATATATGT ATACCTATGA ATGTCAGTAA GTATGTATAC GAACAGTATG
    1861 ATACTGAAGA TGACAAGGTA ATGCATCATT CTATACGTGT CATTCTGAAC GAGGCGCGCT
    1921 TTCCTTTTTT CTTTTTGCTT TTTCTTTTTT TTTCTCTTGA ACTCGACGGA TCTATGCGGT
    1981 GTGAAATACC GCACAGATGC GTCCTAGGGC GGCCGCATAA CTTCGTATAG CATACATTAT
    2041 ACGAAGTTAT CCGGAACCGA GTACAAGCCC ACGGTGCGCC TCGCCACCCG CGACGACGTC
    2101 CCCAGGGCCG TCCGCACCCT CGCCGCCGCG TTCGCCGACT ACCCCGCCAC GCGCCACACC
    2161 GTCGATCCGG ACCGCCACAT CGAGCGCGTG ACCGAGCTGC AAGAACTCTT CCTCACGCGC
    2221 GTCGGGCTCG ACATCGGCAA GGTGTGGGTC GCGGACGACG GAGCCGCTGT GGCGGTCTGG
    2281 ACCACGCCGG AGAGCGTCGA AGCGGGGGCG GTGTTCGCCG AGATCGGCCC GCGCATGGCC
    2341 GAGTTGAGCG GTTCCCGGCT GGCCGCGCAG CAACAGATGG AGGGCCTCCT GGCTCCGCAC
    2401 CGGCCCAAGG AGCCCGCGTG GTTCCTGGCC ACCGTCGGAG TCTCGCCCGA CCACCAGGGC
    2461 AAGGGTCTGG GCAGCGCCGT CGTGCTCCCC GGAGTGGAGG CGGCCGAGCG CGCCGGGGTG
    2521 CCCGCCTTCC TGGAAACCTC CGCGCCCCGC AACCTCCCCT TCTACGAGCG GCTCGGCTTC
    2581 ACCGTCACCG CCGACGTCGA GGTGCCCGAA GGACCGCGCA CCTGGTGCAT GACCCGCAAG
    2641 CCCGGTGCCG CATGCCCCAC GCTACTGCGG GTTTATATAG ACGGTCCCCA CGGGATGGGG
    2701 AAAACCACCA CCACGCAACT GCTGGTGGCC CTGGGTTCGC GCGACGATAT CGTCTATGTA
    2761 CCCGAGCCGA TGACTTACTG GCGGGTGCTG GGGGCTTCCG AGACAATCGC GAACATCTAC
    2821 ACCACACAAC ACCGCCTCGA CCAGGGTGAG ATATCGGCCG GGGACGCGGC GGTGGTAATG
    2881 ACAAGCGCCC AGATAACAAT GGGTATGCCT TATGCCGTGA CCGACGCCGT TCTGGCTCCT
    2941 CATATCGGGG GGGAGGCTGG TAGCTCACAT GCCCCGCCCC CGGCCCTCAC CCTCATCTTC
    3001 GACCGCCATC CCATCGCCGC CCTCCTGTGC TACCCGGCCG CGCGGTACCT TATGGGCAGC
    3061 ATGACCCCCC AGGCCGTGCT GGCGTTCGTG GCCCTCATCC CGCCGACCTT GCCCGGCACC
    3121 AACATCGTGC TTGGGGCCCT GCCGGAGGAC AGACACATCG ACCGCCTGGC CAAACGCCAG
    3181 CGCCCCGGCG AGCGGCTGGA CCTGGCTATG CTGGCTGCGA TTCGCCGCGT TTACGGGCTA
    3241 CTTGCCAATA CGGTGCGGTA TCTGCAGTGC GGCGGGTCGT GGCGGGAGGA CTGGGGACAG
    3301 CTTTCGGGGA CGGCCGTGCC GCCCCAGGGT GCCGAGCCCC AGAGCAACGC GGGCCCACGA
    3361 CCCCATATCG GGGACACGTT ATTTACCCTG TTTCGGGCCC CCGAGTTGCT GGCCCCCAAC
    3421 GGCGACCTGT ATAACGTGTT TGCCTGGGCC TTGGACGTCT TGGCCAAACG CCTCCGTTCC
    3481 ATGCACGTCT TTATCCTGGA TTACGACCAA TCGCCCGCCG GCTGCCGGGA CGCCCTGCTG
    3541 CAACTTACCT CCGGGATGGT CCAGACCCAC GTCACCACCC CCGGCTCCAT ACCGACGATA
    3601 TGCGACCTGG CGCGCACGTT TGCCCGCGAG ATGGGGGAGG CTAACGAGCT CGGCAGTGGA
    3661 GAGGGCAGAG GAAGTCTGCT AACATGCGGT GACGTCGAGG AGAATCCTGG CCCAAGCCAG
    3721 TTCGACATCC TGTGCAAGAC CCCCCCCAAG GTGCTGGTGC GGCAGTTCGT GGAGAGATTC
    3781 GAGAGGCCCA GCGGCGAGAA GATCGCCAGC TGTGCCGCCG AGCTGACCTA CCTGTGCTGG
    3841 ATGATCACCC ACAACGGCAC CGCCATCAAG AGGGCCACCT TCATGAGCTA CAACACCATC
    3901 ATCAGCAACA GCCTGAGCTT CGACATCGTG AACAAGAGCC TGCAGTTCAA GTACAAGACC
    3961 CAGAAGGCCA CCATCCTGGA GGCCAGCCTG AAGAAGCTGA TCCCCGCCTG GGAGTTCACC
    4021 ATCATCCCTT ACAACGGCCA GAAGCACCAG AGCGACATCA CCGACATCGT GTCCAGCCTG
    4081 CAGCTGCAGT TCGAGAGCAG CGAGGAGGCC GACAAGGGCA ACAGCCACAG CAAGAAGATG
    4141 CTGAAGGCCC TGCTGTCCGA GGGCGAGAGC ATCTGGGAGA TCACCGAGAA GATCCTGAAC
    4201 AGCTTCGAGT ACACCAGCAG GTTCACCAAG ACCAAGACCC TGTACCAGTT CCTGTTCCTG
    4261 GCCACATTCA TCAACTGCGG CAGGTTCAGC GACATCAAGA ACGTGGACCC CAAGAGCTTC
    4321 AAGCTGGTGC AGAACAAGTA CCTGGGCGTG ATCATTCAGT GCCTGGTGAC AGAAACCAAG
    4381 ACAAGCGTGT CCAGGCACAT CTACTTTTTC AGCGCCAGAG GCAGGATCGA CCCCCTGGTG
    4441 TACCTGGACG AGTTTCTGAG GAACAGCGAG CCCGTGCTGA AGAGAGTGAA CAGGACCGGC
    4501 AACAGCAGCA GCAACAAGCA GGAGTACCAG CTGCTGAAGG ACAACCTGGT GCGCAGCTAC
    4561 AACAAGGCCC TGAAGAAGAA CGCCCCCTAC CCCATCTTCG CTATCAAGAA CGGCCCTAAG
    4621 AGCCACATCG GCAGGCACCT GATGACCAGC TTTCTGAGCA TGAAGGGCCT GACCGAGCTG
    4681 ACAAACGTGG TGGGCAACTG GAGCGACAAG AGGGCCTCCG CCGTGGCCAG GACCACCTAC
    4741 ACCCACCAGA TCACCGCCAT CCCCGACCAC TACTTCGCCC TGGTGTCCAG GTACTACGCC
    4801 TACGACCCCA TCAGCAAGGA GATGATCGCC CTGAAGGACG AAACCAACCC CATCGAGGAG
    4861 TGGCAGCACA TCGAGCAGCT GAAGGGCAGC GCCGAGGGCA GCATCAGATA CCCCGCCTGG
    4921 AACGGCATCA TCAGCCAGGA GGTGCTGGAC TACCTGAGCA GCTACATCAA CAGGCGGATC
    4981 ACCGGTTGCG TACGCGGATC CTCTGCTGGA GACATGAGAG CTGCCAACCT TTGGCCAAGC
    5041 CCGCTCATGA TCAAACGCTC TAAGAAGAAC AGCCTGGCCT TGTCCCTGAC GGCCGACCAG
    5101 ATGGTCAGTG CCTTGTTGGA TGCTGAGCCC CCCATACTCT ATTCCGAGTA TGATCCTACC
    5161 AGACCCTTCA GTGAAGCTTC GATGATGGGC TTACTGACCA ACCTGGCAGA CAGGGAGCTG
    5221 GTTCACATGA TCAACTGGGC GAAGAGGGTG CCAGGCTTTG TGGATTTGAC CCTCCATGAT
    5281 CAGGTCCACC TTCTAGAATG TGCCTGGCTA GAGATCCTGA TGATTGGTCT CGTCTGGCGC
    5341 TCCATGGAGC ACCCAGTGAA GCTACTGTTT GCTCCTAACT TGCTCTTGGA CAGGAACCAG
    5401 GGAAAATGTG TAGAGGGCAT GGTGGAGATC TTCGACATGC TGCTGGCTAC ATCATCTCGG
    5461 TTCCGCATGA TGAATCTGCA GGGAGAGGAG TTTGTGTGCC TCAAATCTAT TATTTTGCTT
    5521 AATTCTGGAG TGTACACATT TCTGTCCAGC ACCCTGAAGT CTCTGGAAGA GAAGGACCAT
    5581 ATCCACCGAG TCCTGGACAA GATCACAGAC ACTTTGATCC ACCTGATGGC CAAGGCAGGC
    5641 CTGACCCTGC AGCAGCAGCA CCAGCGGCTG GCCCAGCTCC TCCTCATCCT CTCCCACATC
    5701 AGGCACATGA GTAACAAAGG CATGGAGCAT CTGTACAGCA TGAAGTGCAA GAACGTGGTG
    5761 CCCCTCTATG ACCTGCTGCT GGAGGCGGCG GACGCCCACC GCCTACATGC GCCCACTAGC
    5821 CGTGGAGGGG CATCCGTGGA GGAGACGGAC CAAAGCCACT TGGCCACTGC GGGCTCTACT
    5881 TCATCGCATT CCTTGCAAAA GTATTACATC ACGGGGGAGG CAGAGGGTTT CCCTGCCACA
    5941 GCTGGCTCCG GCGAGGGCAG GGGAAGTCTT CTAACATGCG GGGACGTGGA GGAAAATCCC
    6001 GGCCCAGTGA GCAAGGGTGA GGAGGATAAC ATGGCCTCTC TCCCAGCGAC ACATGAGTTA
    6061 CACATCTTTG GCTCCATCAA CGGTGTGGAC TTTGACATGG TGGGTCAGGG CACCGGCAAT
    6121 CCAAATGATG GTTATGAGGA GTTAAACCTG AAGTCCACCA AGGGCGACCT CCAGTTCTCC
    6181 CCCTGGATTC TGGTCCCTCA TATCGGGTAT GGCTTCCATC AGTACCTGCC CTACCCTGAC
    6241 GGGATGTCGC CTTTCCAGGC CGCCATGGTA GATGGCTCTG GATACCAAGT CCATCGCACA
    6301 ATGCAGTTTG AAGATGGTGC CTCCCTTACT GTGAACTACC GCTACACCTA CGAGGGAAGC
    6361 CACATCAAAG GAGAGGCCCA GGTGAAGGGG ACTGGTTTCC CTGCTGACGG TCCTGTGATG
    6421 ACCAACTCGC TGACCGCTGC GGACTGGTGC AGGTCGAAGA AGACTTACCC CAACGACAAA
    6481 ACCATCATCA GTACCTTTAA GTGGAGTTAC ACCACTGGAA ATGGCAAGCG CTACCGGAGC
    6541 ACTGCGCGGA CCACCTACAC CTTTGCCAAG CCAATGGCGG CTAACTATCT GAAGAACCAG
    6601 CCGATGTACG TGTTCCGTAA GACGGAACTC AAGCACTCCA AGACCGAGCT GAACTTCAAG
    6661 GAGTGGCAAA AGGCGTTTAC CGATGTGATG GGCATGGACG AGCTGTACAA GTGAATTCGA
    6721 TCATAATCAG CCATACCACA TTTGTAGAGG TTTTACTTGC TTTAAAAAAC CTCCCACACC
    6781 TCCCCCTGAA CCTGAAACAT AAAATGAATG CAATTGTTGT TGTTAACTTG TTTATTGCAG
    6841 CTTATAATGG TTACAAATAA AGCAATAGCA TCACAAATTT CACAAATAAA GCATTTTTTT
    6901 CACTGCATTC TAGTTGTGGT TTGTCCAAAC TCATCAATGT ATCTTATCAT GTCTGGCTCG
    6961 AGGGAAGTTC CTATACTCTA GAAAGAATAG GAACTTCGGC GGACGCGTAT AGATCAGGCG
    7021 CGCCACAACT TCGTATAGAA AGGTATATAC GAAGTTGTGC GGCCGCGTCG ACCAATTCTC
    7081 ATGTTTGACA GOTTATCATC GAATTTCTGC CATTCATCCG CTTATTATCA CTTATTCAGG
    7141 CGTAGCAACC AGGCGTTTAA GGGCACCAAT AACTGCCTTA AAAAAATTAG AAAAACTCAT
    7201 CGAGCATCAA ATGAAACTGC AATTTATTCA TATCAGGATT ATCAATACCA TATTTTTGAA
    7261 AAAGCCGTTT CTGTAATGAA GGAGAAAACT CACCGAGGCA GTTCCATAGG ATGGCAAGAT
    7321 CCTGGTATCG GTCTGCGATT CCGACTCGTC CAACATCAAT ACAACCTATT AATTTCCCCT
    7381 CGTCAAAAAT AAGGTTATCA AGTGAGAAAT CACCATGAGT GACGACTGAA TCCGGTGAGA
    7441 ATGGCAAAAG CTTATGCATT TCTTTCCAGA CTTGTTCAAC AGGCCAGCCA TTACGCTCGT
    7501 CATCAAAATC ACTCGCATCA ACCAAACCGT TATTCATTCG TGATTGCGCC TGAGCGAGAC
    7561 GAAATACGCG ATCGCTGTTA AAAGGACAAT TACAAACAGG AATCGAATGC AACCGGCGCA
    7621 GGAACACTGC CAGCGCATCA ACAATATTTT CACCTGAATC AGGATATTCT TCTAATACCT
    7681 GGAATGCTGT TTTCCCTGGG ATCGCAGTGG TGAGTAACCA TGCATCATCA GGAGTACGGA
    7741 TAAAATGCTT GATGGTCGGA AGAGGCATAA ATTCCGTCAG CCAGTTTAGT CTGACCATCT
    7801 CATCTGTAAC ATCATTGGCA ACGCTACCTT TGCCATGTTT CAGAAACAAC TCTGGCGCAT
    7861 CGGGCTTCCC ATACAATCGA TAGATTGTCG CACCTGATTG CCCGACATTA TCGCGAGCCC
    7921 ATTTATACCC ATATAAATCA GCATCCATGT TGGAATTTAA TCGCGGCCTC GAGCAAGACG
    7981 TTTCCCGTTG AATATGGCTC ATTTTAGCTT CCTTAGCTCC TGAAAATCTC GATAACTCAA
    8041 AAAATACGCC CGGTAGTGAT CTTATTTCAT TATGGTGAAA GTTGGAACCT CTTACGTGCC
    8101 GATCAACGTC TCATTTTCGC CAAAAGTTGG CCCAGGGCTT CCCGGTATCA ACAGGGACAC
    8161 CAGGATTTAT TTATTCTGCG AAGTGATCTT CCGTCACAGG TATTTATTCG CGATAAGCTC
    8221 ATGGAGCGGC GTAACCGTCG CACAGGAAGG ACAGAGAAAG CGCGGATCTG GGAAGTGACG
    8281 GACAGAACGG TCAGGACCTG GATTGGGGAG GCGGTTGCCG CCGCTGCTGC TGACGGTGTG
    8341 ACGTTCTCTG TTCCGGTCAC ACCACATACG TTCCGCCATT CCTATGCGAT GCACATGCTG
    8401 TATGCCGGTA TACCGCTGAA AGTTCTGCAA AGCCTGATGG GACATAAGTC CATCAGTTCA
    8461 ACGGAAGTCT ACACGAAGGT TTTTGCGCTG GATGTGGCTG CCCGGCACCG GGTGCAGTTT
    8521 GCGATGCCGG AGTCTGATGC GGTTGCGATG CTGAAACAAT TATCCTGAGA ATAAATGCCT
    8581 TGGCCTTTAT ATGGAAATGT GGAACTGAGT GGATATGCTG TTTTTGTCTG TTAAACAGAG
    8641 AAGCTGGCTG TTATCCACTG AGAAGCGAAC GAAACAGTCG GGAAAATCTC CCATTATCGT
    8701 AGAGATCCGC ATTATTAATC TCAGGAGCCT GTGTAGCGTT TATAGGAAGT AGTGTTCTGT
    8761 CATGATGCCT GCAAGCGGTA ACGAAAACGA TTTGAATATG CCTTCAGGAA CAATAGAAAT
    8821 CTTCGTGCGG TGTTACGTTG AAGTGGAGCG GATTATGTCA GCAATGGACA GAACAACCTA
    8881 ATGAACACAG AACCATGATG TGGTCTGTCC TTTTACAGCC AGTAGTGCTC GCCGCAGTCG
    8941 AGCGACAGGG CGAAGCCCTC GAGCTGGTTG CCCTCGCCGC TGGGCTGGCG GCCGTCTATG
    9001 GCCCTGCAAA CGCGCCAGAA ACGCCGTCGA AGCCGTGTGC GAGACACCGC GGCCGGCCGC
    9061 CGGCGTTGTG GATACCTCGC GGAAAACTTG GCCCTCACTG ACAGATGAGG GGCGGACGTT
    9121 GACACTTGAG GGGCCGACTC ACCCGGCGCG GCGTTGACAG ATGAGGGGCA GGCTCGATTT
    9181 CGGCCGGCGA CGTGGAGCTG GCCAGCCTCG CAAATCGGCG AAAACGCCTG ATTTTACGCG
    9241 AGTTTCCCAC AGATGATGTG GACAAGCCTG GGGATAAGTG CCCTGCGGTA TTGACACTTG
    9301 AGGGGCGCGA CTACTGACAG ATGAGGGGCG CGATCCTTGA CACTTGAGGG GCAGAGTGCT
    9361 GACAGATGAG GGGCGCACCT ATTGACATTT GAGGGGCTGT CCACAGGCAG AAAATCCAGC
    9421 ATTTGCAAGG GTTTCCGCCC GTTTTTCGGC CACCGCTAAC CTGTCTTTTA ACCTGCTTTT
    9481 AAACCAATAT TTATAAACCT TGTTTTTAAC CAGGGCTGCG CCCTGTGCGC GTGACCGCGC
    9541 ACGCCGAAGG GGGGTGCCCC CCCTTCTCGA ACCCTCCCGG TCGAGTGAGC GAGGAAGCAC
    9601 CAGGGAACAG CACTTATATA TTCTGCTTAC ACACGATGCC TGAAAAAACT TCCCTTGGGG
    9661 TTATCCACTT ATCCACGGGG ATATTTTTAT AATTATTTTT TTTATAGTTT TTAGATCTTC
    9721 TTTTTTAGAG CGCCTTGTAG GCCTTTATCC ATGCTGGTTC TAGAGAAGGT GTTGTGACAA
    9781 ATTGCCCTTT CAGTGTGACA AATCACCCTC AAATGACAGT CCTGTCTGTG ACAAATTGCC
    9841 CTTAACCCTG TGACAAATTG CCCTCAGAAG AAGCTGTTTT TTCACAAAGT TATCCCTGCT
    9901 TATTGACTCT TTTTTATTTA GTGTGACAAT CTAAAAACTT GTCACACTTC ACATGGATCT
    9961 GTCATGGCGG AAACAGCGGT TATCAATCAC AAGAAACGTA AAAATAGCCC GCGAATCGTC
  10021 CAGTCAAACG ACCTCACTGA GGCGGCATAT AGTCTCTCCC GGGATCAAAA ACGTATGCTG
  10081 TATCTGTTCG TTGACCAGAT CAGAAAATCT GATGGCACCC TACAGGAACA TGACGGTATC
  10141 TGCGAGATCC ATGTTGCTAA ATATGCTGAA ATATTCGGAT TGACCTCTGC GGAAGCCAGT
  10201 AAGGATATAC GGCAGGCATT GAAGAGTTTC GCGGGGAAGG AAGTGGTTTT TTATCGCCCT
  10261 GAAGAGGATG CCGGCGATGA AAAAGGCTAT GAATCTTTTC CTTGGTTTAT CAAACGTGCG
  10321 CACAGTCCAT CCAGAGGGCT TTACAGTGTA CATATCAACC CATATCTCAT TCCCTTCTTT
  10381 ATCGGGTTAC AGAACCGGTT TACGCAGTTT CGGCTTAGTG AAACAAAAGA AATCACCAAT
  10441 CCGTATGCCA TGCGTTTATA CGAATCCCTG TGTCAGTATC GTAAGCCGGA TGGCTCAGGC
  10501 ATCGTCTCTC TGAAAATCGA CTGGATCATA GAGCGTTACC AGCTGCCTCA AAGTTACCAG
  10561 CGTATGCCTG ACTTCCGCCG CCGCTTCCTG CAGGTCTGTG TTAATGAGAT CAACAGCAGA
  10621 ACTCCAATGC GCCTCTCATA CATTGAGAAA AAGAAAGGCC GCCAGACGAC TCATATCGTA
  10681 TTTTCCTTCC GCGATATCAC TTCCATGACG ACAGGATAGT CTGAGGGTTA TCTGTCACAG
  10741 ATTTGAGGGT GGTTCGTCAC ATTTGTTCTG ACCTACTGAG GGTAATTTGT CACAGTTTTG
  10801 CTGTTTCCTT CAGCCTGCAT GGATTTTCTC ATACTTTTTG AACTGTAATT TTTAAGGAAG
  10861 CCAAATTTGA GGGCAGTTTG TCACAGTTGA TTTCCTTCTC TTTCCCTTCG TCATGTGACC
  10921 TGATATCGGG GGTTAGTTCG TCATCATTGA TGAGGGTTGA TTATCACAGT TTATTACTCT
  10981 GAATTGGCTA TCCGCGTGTG TACCTCTACC TGGAGTTTTT CCCACGGTGG ATATTTCTTC
  11041 TTGCGCTGAG CGTAAGAGCT ATCTGACAGA ACAGTTCTTC TTTGCTTCCT CGCCAGTTCG
  11101 CTCGCTATGC TCGGTTACAC GGCTGCGGCG AGCGCTAGTG ATAATAAGTG ACTGAGGTAT
  11161 GTGCTCTTCT TATCTCCTTT TGTAGTGTTG CTCTTATTTT AAACAACTTT GCGGTTTTTT
  11221 GATGACTTTG CGATTTTGTT GTTGCTTTGC AGTAAATTGC AAGATTTAAT AAAAAAACGC
  11281 AAAGCAATGA TTAAAGGATG TTCAGAATGA AACTCATGGA AACACTTAAC CAGTGCATAA
  11341 ACGCTGGTCA TGAAATGACG AAGGCTATCG CCATTGCACA GTTTAATGAT GACAGCCCGG
  11401 AAGCGAGGAA AATAACCCGG CGCTGGAGAA TAGGTGAAGC AGCGGATTTA GTTGGGGTTT
  11461 CTTCTCAGGC TATCAGAGAT GCCGAGAAAG CAGGGCGACT ACCGCACCCG GATATGGAAA
  11521 TTCGAGGACG GGTTGAGCAA CGTGTTGGTT ATACAATTGA ACAAATTAAT CATATGCGTG
  11581 ATGTGTTTGG TACGCGATTG CGACGTGCTG AAGACGTATT TCCACCGGTG ATCGGGGTTG
  11641 CTGCCCATAA AGGTGGCGTT TACAAAACCT CAGTTTCTGT TCATCTTGCT CAGGATCTGG
  11701 CTCTGAAGGG GCTACGTGTT TTGCTCGTGG AAGGTAACGA CCCCCAGGGA ACAGCCTCAA
  11761 TGTATCACGG ATGGGTACCA GATCTTCATA TTCATGCAGA AGACACTCTC CTGCCTTTCT
  11821 ATCTTGGGGA AAAGGACGAT GTCACTTATG CAATAAAGCC CACTTGCTGG CCGGGGCTTG
  11881 ACATTATTCC TTCCTGTCTG GCTCTGCACC GTATTGAAAC TGAGTTAATG GGCAAATTTG
  11941 ATGAAGGTAA ACTGCCCACC GATCCACACC TGATGCTCCG ACTGGCCATT GAAACTGTTG
  12001 CTCATGACTA TGATGTCATA GTTATTGACA GCGCGCCTAA CCTGGGTATC GGCACGATTA
  12061 ATGTCGTATG TGCTGCTGAT GTGCTGATTG TTCCCACGCC TGCTGAGTTG TTTGACTACA
  12121 CCTCCGCACT GCAGTTTTTC GATATGCTTC GTGATCTGCT CAAGAACGTT GATCTTAAAG
  12181 GGTTCGAGCC TGATGTACGT ATTTTGCTTA CCAAATACAG CAATAGCAAT GGCTCTCAGT
  12241 CCCCGTGGAT GGAGGAGCAA ATTCGGGATG CCTGGGGAAG CATGGTTCTA AAAAATGTTG
  12301 TACGTGAAAC GGATGAAGTT GGTAAAGGTC AGATCCGGAT GAGAACTGTT TTTGAACAGG
  12361 CCATTGATCA ACGCTCTTCA ACTGGTGCCT GGAGAAATGC TOTTTCTATT TGGGAACCTG
  12421 TCTGCAATGA AATTTTCGAT CGTCTGATTA AACCACGCTG GGAGATTAGA TAATGAAGCG
  12481 TGCGCCTGTT ATTCCAAAAC ATACGCTCAA TACTCAACCG GTTGAAGATA CTTCGTTATC
  12541 GACACCAGCT GCCCCGATGG TGGATTCGTT AATTGCGCGC GTAGGAGTAA TGGCTCGCGG
  12601 TAATGCCATT ACTTTGCCTG TATGTGGTCG GGATGTGAAG TTTACTCTTG AAGTGCTCCG
  12661 GGGTGATAGT GTTGAGAAGA CCTCTCGGGT ATGGTCAGGT AATGAACGTG ACCAGGAGCT
  12721 GCTTACTGAG GACGCACTGG ATGATCTCAT CCCTTCTTTT CTACTGACTG GTCAACAGAC
  12781 ACCGGCGTTC GGTCGAAGAG TATCTGGTGT CATAGAAATT GCCGATGGGA GTCGCCGTCG
  12841 TAAAGCTGCT GCACTTACCG AAAGTGATTA TCGTGTTCTG GTTGGCGAGC TGGATGATGA
  12901 GCAGATGGCT GCATTATCCA GATTGGGTAA CGATTATCGC CCAACAAGTG CTTATGAACG
  12961 TGGTCAGCGT TATGCAAGCC GATTGCAGAA TGAATTTGCT GGAAATATTT CTGCGCTGGC
  13021 TGATGCGGAA AATATTTCAC GTAAGATTAT TACCCGCTGT ATCAACACCG CCAAATTGCC
  13081 TAAATCAGTT GTTGCTCTTT TTTCTCACCC CGGTGAACTA TCTGCCCGGT CAGGTGATGC
  13141 ACTTCAAAAA GCCTTTACAG ATAAAGAGGA ATTACTTAAG CAGCAGGCAT CTAACCTTCA
  13201 TGAGCAGAAA AAAGCTGGGG TGATATTTGA AGCTGAAGAA GTTATCACTC TTTTAACTTC
  13261 TGTGCTTAAA ACGTCATCTG CATCAAGAAC TAGTTTAAGC TCACGACATC AGTTTGCTCC
  13321 TGGAGCGACA GTATTGTATA AGGGCGATAA AATGGTGCTT AACCTGGACA GGTCTCGTGT
  13381 TCCAACTGAG TGTATAGAGA AAATTGAGGC CATTCTTAAG GAACTTGAAA AGCCAGCACC
  13441 CTGATGCGAC CACGTTTTAG TCTACGTTTA TCTGTCTTTA CTTAATGTCC TTTGTTACAG
  13501 GCCAGAAAGC ATAACTGGCC TGAATATTCT CTCTGGGCCC ACTGTTCCAC TTGTATCGTC
  13561 GGTCTGATAA TCAGACTGGG ACCACGGTCC CACTCGTATC GTCGGTCTGA TTATTAGTCT
  13621 GGGACCACGG TCCCACTCGT ATCGTCGGTC TGATTATTAG TCTGGGACCA CGGTCCCACT
  13681 CGTATCGTCG GTCTGATAAT CAGACTGGGA CCACGGTCCC ACTCGTATCG TCGGTCTGAT
  13741 TATTAGTCTG GGACCATGGT CCCACTCGTA TCGTCGGTCT GATTATTAGT CTGGGACCAC
  13801 GGTCCCACTC GTATCGTCGG TCTGATTATT AGTCTGGAAC CACGGTCCCA CTCGTATCGT
  13861 CGGTCTGATT ATTAGTCTGG GACCACGGTC CCACTCGTAT CGTCGGTCTG ATTATTAGTC
  13921 TGGGACCACG ATCCCACTCG TGTTGTCGGT CTGATTATCG GTCTGGGACC ACGGTCCCAC
  13981 TTGTATTGTC GATCAGACTA TCAGCGTGAG ACTACGATTC CATCAATGCC TGTCAAGGGC
  14041 AAGTATTGAC ATGTCGTCGT AACCTGTAGA ACGGAGTAAC CTCGGTGTGC GGTTGTATGC
  14101 CTGCTGTGGA TTGCTGCTGT GTCCTGCTTA TCCACAACAT TTTGCGCACG GTTATGTGGA
  14161 C
(Payload Vector Exit2_FlpOUTneo)
SEQ ID NO: 12
          1 CCCCGAAAAG TGCCACCTGG GTCCTTTTCA TCACGTGCTA TAAAAATAAT TATAATTTAA
        61 ATTTTTTAAT ATAAATATAT AAATTAAAAA TAGAAAGTAA AAAAAGAAAT TAAAGAAAAA
      121 ATAGTTTTTG TTTTCCGAAG ATGTAAAAGA CTCTAGGGGG ATCGCCAACA AATACTACCT
      181 TTTATCTTGC TCTTCCTGCT CTCAGGTATT AATGCCGAAT TGTTTCATCT TGTCTGTGTA
      241 GAAGACCACA CACGAAAATC CTGTGATTTT ACATTTTACT TATCGTTAAT CGAATGTATA
      301 TCTATTTAAT CTGCTTTTCT TGTCTAATAA ATATATATGT AAAGTACGCT TTTTGTTGAA
      361 ATTTTTTAAA CCTTTGTTTA TTTTTTTTTC TTCATTCCGT AACTCTTCTA CCTTCTTTAT
      421 TTACTTTCTA AAATCCAAAT ACAAAACATA AAAATAAATA AACACAGAGT AAATTCCCAA
      481 ATTATTCCAT CATTAAAAGA TACGAGGCGC GTGTAAGTTA CAGGCAAGCG ATCCGTCCTA
      541 AGAAACCATT ATTATCATGA CATTAACCTA TAAAAATAGG CGTATCACGA GGCCCTTTCG
      601 TCTCGCGCGT TTCGGTGATG ACGGTGAAAA CCTCTGACAC ATGCAGCTCC CGGAGACGGT
      661 CACAGOTTGT CTGTAAGCGG ATGCCGGGAG CAGACAAGCC CGTCAGGGCG CGTCAGCGCG
      721 TGTTGGCGGG TGTCGGGGCT GGCTTAACTA TGCGGCATCA GAGCAGATTG TACTGAGAGT
      781 GCACCATAAA TTCCCGTTTT AAGAGCTTGG TGAGCGCTAG GAGTCACTGC CAGGTATCGT
      841 TTGAACACGG CATTAGTCAG GGAAGTCATA ACACAGTCCT TTCCCGCAAT TTTCTTTTTC
      901 TATTACTCTT GGCCTCCTCT AGTACACTCT ATATTTTTTT ATGCCTCGGT AATGATTTTC
      961 ATTTTTTTTT TTCCCCTAGC GGATGACTCT TTTTTTTTCT TAGCGATTGG CATTATCACA
    1021 TAATGAATTA TACATTATAT AAAGTAATGT GATTTCTTCG AAGAATATAC TAAAAAATGA
    1081 GCAGGCAAGA TAAACGAAGG CAAAGATGAC AGAGCAGAAA GCCCTAGTAA AGCGTATTAC
    1141 AAATGAAACC AAGATTCAGA TTGCGATCTC TTTAAAGGGT GGTCCCCTAG CGATAGAGCA
    1201 CTCGATCTTC CCAGAAAAAG AGGCAGAAGC AGTAGCAGAA CAGGCCACAC AATCGCAAGT
    1261 GATTAACGTC CACACAGGTA TAGGGTTTCT GGACCATATG ATACATGCTC TGGCCAAGCA
    1321 TTCCGGCTGG TCGCTAATCG TTGAGTGCAT TGGTGACTTA CACATAGACG ACCATCACAC
    1381 CACTGAAGAC TGCGGGATTG CTCTCGGTCA AGCTTTTAAA GAGGCCCTAC TGGCGCGTGG
    1441 AGTAAAAAGG TTTGGATCAG GATTTGCGCC TTTGGATGAG GCACTTTCCA GAGCGGTGGT
    1501 AGATCTTTCG AACAGGCCGT ACGCAGTTGT CGAACTTGGT TTGCAAAGGG AGAAAGTAGG
    1561 AGATCTCTCT TGCGAGATGA TCCCGCATTT TCTTGAAAGC TTTGCAGAGG CTAGCAGAAT
    1621 TACCCTCCAC GTTGATTGTC TGCGAGGCAA GAATGATCAT CACCGTAGTG AGAGTGCGTT
    1681 CAAGGCTCTT GCGGTTGCCA TAAGAGAAGC CACCTCGCCC AATGGTACCA ACGATGTTCC
    1741 CTCCACCAAA GGTGTTCTTA TGTAGTGACA CCGATTATTT AAAGCTGCAG CATACGATAT
    1801 ATATACATGT GTATATATGT ATACCTATGA ATGTCAGTAA GTATGTATAC GAACAGTATG
    1861 ATACTGAAGA TGACAAGGTA ATGCATCATT CTATACGTGT CATTCTGAAC GAGGCGCGCT
    1921 TTCCTTTTTT CTTTTTGCTT TTTCTTTTTT TTTCTCTTGA ACTCGACGGA TCTATGCGGT
    1981 GTGAAATACC GCACAGATGC GTCCTAGGGC GGCCGCATAA CTTCGTATAG CATACATTAT
    2041 ACGAAGTTAT CCGGAAAAAC ATTTAACATT TCTCAACAAG ATCTAGAATT AGTAGAAGTA
    2101 GCGACAGAGA AGATTACAAT GOTTTATGAG GATAATAAAC ATCATGTGGG AGCGGCAATT
    2161 CGTACGAAAA CAGGAGAAAT CATTTCGGCA GTACATATTG AAGCGTATAT AGGACGAGTA
    2221 ACTGTTTGTG CAGAAGCCAT TGCGATTGGT AGTGCAGTTT CGAATGGACA AAAGGATTTT
    2281 GACACGATTG TAGCTGTTAG ACACCCTTAT TCTGACGAAG TAGATAGAAG TATTCGAGTG
    2341 GTAAGTCCTT GTGGTATGTG TAGGGAGTTG ATTTCAGACT ATGCACCAGA TTGTTTTGTG
    2401 TTAATAGAAA TGAATGGCAA GTTAGTCAAA ACTACGATTG AAGAACTCAT TCCACTCAAA
    2461 TATACCCGAA ATGCATGCCC CACGCTACTG CGGGTTTATA TAGACGGTCC CCACGGGATG
    2521 GGGAAAACCA CCACCACGCA ACTGCTGGTG GCCCTGGGTT CGCGCGACGA TATCGTCTAT
    2581 GTACCCGAGC CGATGACTTA CTGGCGGGTG CTGGGGGCTT CCGAGACAAT CGCGAACATC
    2641 TACACCACAC AACACCGCCT CGACCAGGGT GAGATATCGG CCGGGGACGC GGCGGTGGTA
    2701 ATGACAAGCG CCCAGATAAC AATGGGTATG CCTTATGCCG TGACCGACGC CGTTCTGGCT
    2761 CCTCATATCG GGGGGGAGGC TGGTAGCTCA CATGCCCCGC CCCCGGCCCT CACCCTCATC
    2821 TTCGACCGCC ATCCCATCGC CGCCCTCCTG TGCTACCCGG CCGCGCGGTA CCTTATGGGC
    2881 AGCATGACCC CCCAGGCCGT GCTGGCGTTC GTGGCCCTCA TCCCGCCGAC CTTGCCCGGC
    2941 ACCAACATCG TGCTTGGGGC CCTGCCGGAG GACAGACACA TCGACCGCCT GGCCAAACGC
    3001 CAGCGCCCCG GCGAGCGGCT GGACCTGGCT ATGCTGGCTG CGATTCGCCG CGTTTACGGG
    3061 CTACTTGCCA ATACGGTGCG GTATCTGCAG TGCGGCGGGT CGTGGCGGGA GGACTGGGGA
    3121 CAGCTTTCGG GGACGGCCGT GCCGCCCCAG GGTGCCGAGC CCCAGAGCAA CGCGGGCCCA
    3181 CGACCCCATA TCGGGGACAC GTTATTTACC CTGTTTCGGG CCCCCGAGTT GCTGGCCCCC
    3241 AACGGCGACC TGTATAACGT GTTTGCCTGG GCCTTGGACG TCTTGGCCAA ACGCCTCCGT
    3301 TCCATGCACG TCTTTATCCT GGATTACGAC CAATCGCCCG CCGGCTGCCG GGACGCCCTG
    3361 CTGCAACTTA CCTCCGGGAT GGTCCAGACC CACGTCACCA CCCCCGGCTC CATACCGACG
    3421 ATATGCGACC TGGCGCGCAC GTTTGCCCGC GAGATGGGGG AGGCTAACGA GCTCGGCAGT
    3481 GGAGAGGGCA GAGGAAGTCT GCTAACATGC GGTGACGTCG AGGAGAATCC TGGCCCAAGC
    3541 CAGTTCGACA TCCTGTGCAA GACCCCCCCC AAGGTGCTGG TGCGGCAGTT CGTGGAGAGA
    3601 TTCGAGAGGC CCAGCGGCGA GAAGATCGCC AGCTGTGCCG CCGAGCTGAC CTACCTGTGC
    3661 TGGATGATCA CCCACAACGG CACCGCCATC AAGAGGGCCA CCTTCATGAG CTACAACACC
    3721 ATCATCAGCA ACAGCCTGAG CTTCGACATC GTGAACAAGA GCCTGCAGTT CAAGTACAAG
    3781 ACCCAGAAGG CCACCATCCT GGAGGCCAGC CTGAAGAAGC TGATCCCCGC CTGGGAGTTC
    3841 ACCATCATCC CTTACAACGG CCAGAAGCAC CAGAGCGACA TCACCGACAT CGTGTCCAGC
    3901 CTGCAGCTGC AGTTCGAGAG CAGCGAGGAG GCCGACAAGG GCAACAGCCA CAGCAAGAAG
    3961 ATGCTGAAGG CCCTGCTGTC CGAGGGCGAG AGCATCTGGG AGATCACCGA GAAGATCCTG
    4021 AACAGCTTCG AGTACACCAG CAGGTTCACC AAGACCAAGA CCCTGTACCA GTTCCTGTTC
    4081 CTGGCCACAT TCATCAACTG CGGCAGGTTC AGCGACATCA AGAACGTGGA CCCCAAGAGC
    4141 TTCAAGCTGG TGCAGAACAA GTACCTGGGC GTGATCATTC AGTGCCTGGT GACAGAAACC
    4201 AAGACAAGCG TGTCCAGGCA CATCTACTTT TTCAGCGCCA GAGGCAGGAT CGACCCCCTG
    4261 GTGTACCTGG ACGAGTTTCT GAGGAACAGC GAGCCCGTGC TGAAGAGAGT GAACAGGACC
    4321 GGCAACAGCA GCAGCAACAA GCAGGAGTAC CAGCTGCTGA AGGACAACCT GGTGCGCAGC
    4381 TACAACAAGG CCCTGAAGAA GAACGCCCCC TACCCCATCT TCGCTATCAA GAACGGCCCT
    4441 AAGAGCCACA TCGGCAGGCA CCTGATGACC AGCTTTCTGA GCATGAAGGG CCTGACCGAG
    4501 CTGACAAACG TGGTGGGCAA CTGGAGCGAC AAGAGGGCCT CCGCCGTGGC CAGGACCACC
    4561 TACACCCACC AGATCACCGC CATCCCCGAC CACTACTTCG CCCTGGTGTC CAGGTACTAC
    4621 GCCTACGACC CCATCAGCAA GGAGATGATC GCCCTGAAGG ACGAAACCAA CCCCATCGAG
    4681 GAGTGGCAGC ACATCGAGCA GCTGAAGGGC AGCGCCGAGG GCAGCATCAG ATACCCCGCC
    4741 TGGAACGGCA TCATCAGCCA GGAGGTGCTG GACTACCTGA GCAGCTACAT CAACAGGCGG
    4801 ATCACCGGTT GCGTACGCGG ATCCTCTGCT GGAGACATGA GAGCTGCCAA CCTTTGGCCA
    4861 AGCCCGCTCA TGATCAAACG CTCTAAGAAG AACAGCCTGG CCTTGTCCCT GACGGCCGAC
    4921 CAGATGGTCA GTGCCTTGTT GGATGCTGAG CCCCCCATAC TCTATTCCGA GTATGATCCT
    4981 ACCAGACCCT TCAGTGAAGC TTCGATGATG GGCTTACTGA CCAACCTGGC AGACAGGGAG
    5041 CTGGTTCACA TGATCAACTG GGCGAAGAGG GTGCCAGGCT TTGTGGATTT GACCCTCCAT
    5101 GATCAGGTCC ACCTTCTAGA ATGTGCCTGG CTAGAGATCC TGATGATTGG TCTCGTCTGG
    5161 CGCTCCATGG AGCACCCAGT GAAGCTACTG TTTGCTCCTA ACTTGCTCTT GGACAGGAAC
    5221 CAGGGAAAAT GTGTAGAGGG CATGGTGGAG ATCTTCGACA TGCTGCTGGC TACATCATCT
    5281 CGGTTCCGCA TGATGAATCT GCAGGGAGAG GAGTTTGTGT GCCTCAAATC TATTATTTTG
    5341 CTTAATTCTG GAGTGTACAC ATTTCTGTCC AGCACCCTGA AGTCTCTGGA AGAGAAGGAC
    5401 CATATCCACC GAGTCCTGGA CAAGATCACA GACACTTTGA TCCACCTGAT GGCCAAGGCA
    5461 GGCCTGACCC TGCAGCAGCA GCACCAGCGG CTGGCCCAGC TCCTCCTCAT CCTCTCCCAC
    5521 ATCAGGCACA TGAGTAACAA AGGCATGGAG CATCTGTACA GCATGAAGTG CAAGAACGTG
    5581 GTGCCCCTCT ATGACCTGCT GCTGGAGGCG GCGGACGCCC ACCGCCTACA TGCGCCCACT
    5641 AGCCGTGGAG GGGCATCCGT GGAGGAGACG GACCAAAGCC ACTTGGCCAC TGCGGGCTCT
    5701 ACTTCATCGC ATTCCTTGCA AAAGTATTAC ATCACGGGGG AGGCAGAGGG TTTCCCTGCC
    5761 ACAGCTGGCT CCGGCGAGGG CAGGGGAAGT CTTCTAACAT GCGGGGACGT GGAGGAAAAT
    5821 CCCGGCCCAG TGAGCAAGGG CGAGGCAGTG ATCAAGGAGT TCATGCGGTT CAAGGTGCAC
    5881 ATGGAGGGCT CCATGAACGG CCACGAGTTC GAGATCGAGG GCGAGGGCGA GGGCCGCCCC
    5941 TACGAGGGCA CCCAGACCGC CAAGCTGAAG GTGACCAAGG GTGGCCCCCT GCCCTTCTCC
    6001 TGGGACATCC TGTCCCCTCA GTTCATGTAC GGCTCCAGGG CCTTCACCAA GCACCCCGCC
    6061 GACATCCCCG ACTACTATAA GCAGTCCTTC CCCGAGGGCT TCAAGTGGGA GCGCGTGATG
    6121 AACTTCGAGG ACGGCGGCGC CGTGACCGTG ACCCAGGACA CCTCCCTGGA GGACGGCACC
    6181 CTGATCTACA AGGTGAAGCT CCGCGGCACC AACTTCCCTC CTGACGGCCC CGTAATGCAG
    6241 AAGAAAACAA TGGGCTGGGA AGCGTCCACC GAGCGGTTGT ACCCCGAGGA CGGCGTGCTG
    6301 AAGGGCGACA TTAAGATGGC CCTGCGCCTG AAGGACGGCG GCCGCTACCT GGCGGACTTC
    6361 AAGACCACCT ACAAGGCCAA GAAGCCCGTG CAGATGCCCG GCGCCTACAA CGTTGACCGC
    6421 AAGTTGGACA TCACCTCCCA CAACGAGGAC TACACCGTGG TGGAACAGTA CGAACGCTCC
    6481 GAGGGCCGCC ACTCCACCGG CGGCATGGAC GAGCTGTACA AGTGAATTCG ATCATAATCA
    6541 GCCATACCAC ATTTGTAGAG GTTTTACTTG CTTTAAAAAA CCTCCCACAC CTCCCCCTGA
    6601 ACCTGAAACA TAAAATGAAT GCAATTGTTG TTGTTAACTT GTTTATTGCA GCTTATAATG
    6661 GTTACAAATA AAGCAATAGC ATCACAAATT TCACAAATAA AGCATTTTTT TCACTGCATT
    6721 CTAGTTGTGG TTTGTCCAAA CTCATCAATG TATCTTATCA TGTCTGGCTC GAGGGAAGTT
    6781 CCTATACTCT AGAAAGAATA GGAACTTCGG CGGACGCGTA TAGATCAGGC GCGCCATAAC
    6841 TTCGTATAGT ATAGTATATA CGAATTATGC GGCCGCGTCG ACCAATTCTC ATGTTTGACA
    6901 GOTTATCATC GAATTTCTGC CATTCATCCG CTTATTATCA CTTATTCAGG CGTAGCAACC
    6961 AGGCGTTTAA GGGCACCAAT AACTGCCTTA AAAAAATTAG AAAAACTCAT CGAGCATCAA
    7021 ATGAAACTGC AATTTATTCA TATCAGGATT ATCAATACCA TATTTTTGAA AAAGCCGTTT
    7081 CTGTAATGAA GGAGAAAACT CACCGAGGCA GTTCCATAGG ATGGCAAGAT CCTGGTATCG
    7141 GTCTGCGATT CCGACTCGTC CAACATCAAT ACAACCTATT AATTTCCCCT CGTCAAAAAT
    7201 AAGGTTATCA AGTGAGAAAT CACCATGAGT GACGACTGAA TCCGGTGAGA ATGGCAAAAG
    7261 CTTATGCATT TCTTTCCAGA CTTGTTCAAC AGGCCAGCCA TTACGCTCGT CATCAAAATC
    7321 ACTCGCATCA ACCAAACCGT TATTCATTCG TGATTGCGCC TGAGCGAGAC GAAATACGCG
    7381 ATCGCTGTTA AAAGGACAAT TACAAACAGG AATCGAATGC AACCGGCGCA GGAACACTGC
    7441 CAGCGCATCA ACAATATTTT CACCTGAATC AGGATATTCT TCTAATACCT GGAATGCTGT
    7501 TTTCCCTGGG ATCGCAGTGG TGAGTAACCA TGCATCATCA GGAGTACGGA TAAAATGCTT
    7561 GATGGTCGGA AGAGGCATAA ATTCCGTCAG CCAGTTTAGT CTGACCATCT CATCTGTAAC
    7621 ATCATTGGCA ACGCTACCTT TGCCATGTTT CAGAAACAAC TCTGGCGCAT CGGGCTTCCC
    7681 ATACAATCGA TAGATTGTCG CACCTGATTG CCCGACATTA TCGCGAGCCC ATTTATACCC
    7741 ATATAAATCA GCATCCATGT TGGAATTTAA TCGCGGCCTC GAGCAAGACG TTTCCCGTTG
    7801 AATATGGCTC ATTTTAGCTT CCTTAGCTCC TGAAAATCTC GATAACTCAA AAAATACGCC
    7861 CGGTAGTGAT CTTATTTCAT TATGGTGAAA GTTGGAACCT CTTACGTGCC GATCAACGTC
    7921 TCATTTTCGC CAAAAGTTGG CCCAGGGCTT CCCGGTATCA ACAGGGACAC CAGGATTTAT
    7981 TTATTCTGCG AAGTGATCTT CCGTCACAGG TATTTATTCG CGATAAGCTC ATGGAGCGGC
    8041 GTAACCGTCG CACAGGAAGG ACAGAGAAAG CGCGGATCTG GGAAGTGACG GACAGAACGG
    8101 TCAGGACCTG GATTGGGGAG GCGGTTGCCG CCGCTGCTGC TGACGGTGTG ACGTTCTCTG
    8161 TTCCGGTCAC ACCACATACG TTCCGCCATT CCTATGCGAT GCACATGCTG TATGCCGGTA
    8221 TACCGCTGAA AGTTCTGCAA AGCCTGATGG GACATAAGTC CATCAGTTCA ACGGAAGTCT
    8281 ACACGAAGGT TTTTGCGCTG GATGTGGCTG CCCGGCACCG GGTGCAGTTT GCGATGCCGG
    8341 AGTCTGATGC GGTTGCGATG CTGAAACAAT TATCCTGAGA ATAAATGCCT TGGCCTTTAT
    8401 ATGGAAATGT GGAACTGAGT GGATATGCTG TTTTTGTCTG TTAAACAGAG AAGCTGGCTG
    8461 TTATCCACTG AGAAGCGAAC GAAACAGTCG GGAAAATCTC CCATTATCGT AGAGATCCGC
    8521 ATTATTAATC TCAGGAGCCT GTGTAGCGTT TATAGGAAGT AGTGTTCTGT CATGATGCCT
    8581 GCAAGCGGTA ACGAAAACGA TTTGAATATG CCTTCAGGAA CAATAGAAAT CTTCGTGCGG
    8641 TGTTACGTTG AAGTGGAGCG GATTATGTCA GCAATGGACA GAACAACCTA ATGAACACAG
    8701 AACCATGATG TGGTCTGTCC TTTTACAGCC AGTAGTGCTC GCCGCAGTCG AGCGACAGGG
    8761 CGAAGCCCTC GAGCTGGTTG CCCTCGCCGC TGGGCTGGCG GCCGTCTATG GCCCTGCAAA
    8821 CGCGCCAGAA ACGCCGTCGA AGCCGTGTGC GAGACACCGC GGCCGGCCGC CGGCGTTGTG
    8881 GATACCTCGC GGAAAACTTG GCCCTCACTG ACAGATGAGG GGCGGACGTT GACACTTGAG
    8941 GGGCCGACTC ACCCGGCGCG GCGTTGACAG ATGAGGGGCA GGCTCGATTT CGGCCGGCGA
    9001 CGTGGAGCTG GCCAGCCTCG CAAATCGGCG AAAACGCCTG ATTTTACGCG AGTTTCCCAC
    9061 AGATGATGTG GACAAGCCTG GGGATAAGTG CCCTGCGGTA TTGACACTTG AGGGGCGCGA
    9121 CTACTGACAG ATGAGGGGCG CGATCCTTGA CACTTGAGGG GCAGAGTGCT GACAGATGAG
    9181 GGGCGCACCT ATTGACATTT GAGGGGCTGT CCACAGGCAG AAAATCCAGC ATTTGCAAGG
    9241 GTTTCCGCCC GTTTTTCGGC CACCGCTAAC CTGTCTTTTA ACCTGCTTTT AAACCAATAT
    9301 TTATAAACCT TGTTTTTAAC CAGGGCTGCG CCCTGTGCGC GTGACCGCGC ACGCCGAAGG
    9361 GGGGTGCCCC CCCTTCTCGA ACCCTCCCGG TCGAGTGAGC GAGGAAGCAC CAGGGAACAG
    9421 CACTTATATA TTCTGCTTAC ACACGATGCC TGAAAAAACT TCCCTTGGGG TTATCCACTT
    9481 ATCCACGGGG ATATTTTTAT AATTATTTTT TTTATAGTTT TTAGATCTTC TTTTTTAGAG
    9541 CGCCTTGTAG GCCTTTATCC ATGCTGGTTC TAGAGAAGGT GTTGTGACAA ATTGCCCTTT
    9601 CAGTGTGACA AATCACCCTC AAATGACAGT CCTGTCTGTG ACAAATTGCC CTTAACCCTG
    9661 TGACAAATTG CCCTCAGAAG AAGCTGTTTT TTCACAAAGT TATCCCTGCT TATTGACTCT
    9721 TTTTTATTTA GTGTGACAAT CTAAAAACTT GTCACACTTC ACATGGATCT GTCATGGCGG
    9781 AAACAGCGGT TATCAATCAC AAGAAACGTA AAAATAGCCC GCGAATCGTC CAGTCAAACG
    9841 ACCTCACTGA GGCGGCATAT AGTCTCTCCC GGGATCAAAA ACGTATGCTG TATCTGTTCG
    9901 TTGACCAGAT CAGAAAATCT GATGGCACCC TACAGGAACA TGACGGTATC TGCGAGATCC
    9961 ATGTTGCTAA ATATGCTGAA ATATTCGGAT TGACCTCTGC GGAAGCCAGT AAGGATATAC
  10021 GGCAGGCATT GAAGAGTTTC GCGGGGAAGG AAGTGGTTTT TTATCGCCCT GAAGAGGATG
  10081 CCGGCGATGA AAAAGGCTAT GAATCTTTTC CTTGGTTTAT CAAACGTGCG CACAGTCCAT
  10141 CCAGAGGGCT TTACAGTGTA CATATCAACC CATATCTCAT TCCCTTCTTT ATCGGGTTAC
  10201 AGAACCGGTT TACGCAGTTT CGGCTTAGTG AAACAAAAGA AATCACCAAT CCGTATGCCA
  10261 TGCGTTTATA CGAATCCCTG TGTCAGTATC GTAAGCCGGA TGGCTCAGGC ATCGTCTCTC
  10321 TGAAAATCGA CTGGATCATA GAGCGTTACC AGCTGCCTCA AAGTTACCAG CGTATGCCTG
  10381 ACTTCCGCCG CCGCTTCCTG CAGGTCTGTG TTAATGAGAT CAACAGCAGA ACTCCAATGC
  10441 GCCTCTCATA CATTGAGAAA AAGAAAGGCC GCCAGACGAC TCATATCGTA TTTTCCTTCC
  10501 GCGATATCAC TTCCATGACG ACAGGATAGT CTGAGGGTTA TCTGTCACAG ATTTGAGGGT
  10561 GGTTCGTCAC ATTTGTTCTG ACCTACTGAG GGTAATTTGT CACAGTTTTG CTGTTTCCTT
  10621 CAGCCTGCAT GGATTTTCTC ATACTTTTTG AACTGTAATT TTTAAGGAAG CCAAATTTGA
  10681 GGGCAGTTTG TCACAGTTGA TTTCCTTCTC TTTCCCTTCG TCATGTGACC TGATATCGGG
  10741 GGTTAGTTCG TCATCATTGA TGAGGGTTGA TTATCACAGT TTATTACTCT GAATTGGCTA
  10801 TCCGCGTGTG TACCTCTACC TGGAGTTTTT CCCACGGTGG ATATTTCTTC TTGCGCTGAG
  10861 CGTAAGAGCT ATCTGACAGA ACAGTTCTTC TTTGCTTCCT CGCCAGTTCG CTCGCTATGC
  10921 TCGGTTACAC GGCTGCGGCG AGCGCTAGTG ATAATAAGTG ACTGAGGTAT GTGCTCTTCT
  10981 TATCTCCTTT TGTAGTGTTG CTCTTATTTT AAACAACTTT GCGGTTTTTT GATGACTTTG
  11041 CGATTTTGTT GTTGCTTTGC AGTAAATTGC AAGATTTAAT AAAAAAACGC AAAGCAATGA
  11101 TTAAAGGATG TTCAGAATGA AACTCATGGA AACACTTAAC CAGTGCATAA ACGCTGGTCA
  11161 TGAAATGACG AAGGCTATCG CCATTGCACA GTTTAATGAT GACAGCCCGG AAGCGAGGAA
  11221 AATAACCCGG CGCTGGAGAA TAGGTGAAGC AGCGGATTTA GTTGGGGTTT CTTCTCAGGC
  11281 TATCAGAGAT GCCGAGAAAG CAGGGCGACT ACCGCACCCG GATATGGAAA TTCGAGGACG
  11341 GGTTGAGCAA CGTGTTGGTT ATACAATTGA ACAAATTAAT CATATGCGTG ATGTGTTTGG
  11401 TACGCGATTG CGACGTGCTG AAGACGTATT TCCACCGGTG ATCGGGGTTG CTGCCCATAA
  11461 AGGTGGCGTT TACAAAACCT CAGTTTCTGT TCATCTTGCT CAGGATCTGG CTCTGAAGGG
  11521 GCTACGTGTT TTGCTCGTGG AAGGTAACGA CCCCCAGGGA ACAGCCTCAA TGTATCACGG
  11581 ATGGGTACCA GATCTTCATA TTCATGCAGA AGACACTCTC CTGCCTTTCT ATCTTGGGGA
  11641 AAAGGACGAT GTCACTTATG CAATAAAGCC CACTTGCTGG CCGGGGCTTG ACATTATTCC
  11701 TTCCTGTCTG GCTCTGCACC GTATTGAAAC TGAGTTAATG GGCAAATTTG ATGAAGGTAA
  11761 ACTGCCCACC GATCCACACC TGATGCTCCG ACTGGCCATT GAAACTGTTG CTCATGACTA
  11821 TGATGTCATA GTTATTGACA GCGCGCCTAA CCTGGGTATC GGCACGATTA ATGTCGTATG
  11881 TGCTGCTGAT GTGCTGATTG TTCCCACGCC TGCTGAGTTG TTTGACTACA CCTCCGCACT
  11941 GCAGTTTTTC GATATGCTTC GTGATCTGCT CAAGAACGTT GATCTTAAAG GGTTCGAGCC
  12001 TGATGTACGT ATTTTGCTTA CCAAATACAG CAATAGCAAT GGCTCTCAGT CCCCGTGGAT
  12061 GGAGGAGCAA ATTCGGGATG CCTGGGGAAG CATGGTTCTA AAAAATGTTG TACGTGAAAC
  12121 GGATGAAGTT GGTAAAGGTC AGATCCGGAT GAGAACTGTT TTTGAACAGG CCATTGATCA
  12181 ACGCTCTTCA ACTGGTGCCT GGAGAAATGC TCTTTCTATT TGGGAACCTG TCTGCAATGA
  12241 AATTTTCGAT CGTCTGATTA AACCACGCTG GGAGATTAGA TAATGAAGCG TGCGCCTGTT
  12301 ATTCCAAAAC ATACGCTCAA TACTCAACCG GTTGAAGATA CTTCGTTATC GACACCAGCT
  12361 GCCCCGATGG TGGATTCGTT AATTGCGCGC GTAGGAGTAA TGGCTCGCGG TAATGCCATT
  12421 ACTTTGCCTG TATGTGGTCG GGATGTGAAG TTTACTCTTG AAGTGCTCCG GGGTGATAGT
  12481 GTTGAGAAGA CCTCTCGGGT ATGGTCAGGT AATGAACGTG ACCAGGAGCT GCTTACTGAG
  12541 GACGCACTGG ATGATCTCAT CCCTTCTTTT CTACTGACTG GTCAACAGAC ACCGGCGTTC
  12601 GGTCGAAGAG TATCTGGTGT CATAGAAATT GCCGATGGGA GTCGCCGTCG TAAAGCTGCT
  12661 GCACTTACCG AAAGTGATTA TCGTGTTCTG GTTGGCGAGC TGGATGATGA GCAGATGGCT
  12721 GCATTATCCA GATTGGGTAA CGATTATCGC CCAACAAGTG CTTATGAACG TGGTCAGCGT
  12781 TATGCAAGCC GATTGCAGAA TGAATTTGCT GGAAATATTT CTGCGCTGGC TGATGCGGAA
  12841 AATATTTCAC GTAAGATTAT TACCCGCTGT ATCAACACCG CCAAATTGCC TAAATCAGTT
  12901 GTTGCTCTTT TTTCTCACCC CGGTGAACTA TCTGCCCGGT CAGGTGATGC ACTTCAAAAA
  12961 GCCTTTACAG ATAAAGAGGA ATTACTTAAG CAGCAGGCAT CTAACCTTCA TGAGCAGAAA
  13021 AAAGCTGGGG TGATATTTGA AGCTGAAGAA GTTATCACTC TTTTAACTTC TGTGCTTAAA
  13081 ACGTCATCTG CATCAAGAAC TAGTTTAAGC TCACGACATC AGTTTGCTCC TGGAGCGACA
  13141 GTATTGTATA AGGGCGATAA AATGGTGCTT AACCTGGACA GGTCTCGTGT TCCAACTGAG
  13201 TGTATAGAGA AAATTGAGGC CATTCTTAAG GAACTTGAAA AGCCAGCACC CTGATGCGAC
  13261 CACGTTTTAG TCTACGTTTA TCTGTCTTTA CTTAATGTCC TTTGTTACAG GCCAGAAAGC
  13321 ATAACTGGCC TGAATATTCT CTCTGGGCCC ACTGTTCCAC TTGTATCGTC GGTCTGATAA
  13381 TCAGACTGGG ACCACGGTCC CACTCGTATC GTCGGTCTGA TTATTAGTCT GGGACCACGG
  13441 TCCCACTCGT ATCGTCGGTC TGATTATTAG TCTGGGACCA CGGTCCCACT CGTATCGTCG
  13501 GTCTGATAAT CAGACTGGGA CCACGGTCCC ACTCGTATCG TCGGTCTGAT TATTAGTCTG
  13561 GGACCATGGT CCCACTCGTA TCGTCGGTCT GATTATTAGT CTGGGACCAC GGTCCCACTC
  13621 GTATCGTCGG TCTGATTATT AGTCTGGAAC CACGGTCCCA CTCGTATCGT CGGTCTGATT
  13681 ATTAGTCTGG GACCACGGTC CCACTCGTAT CGTCGGTCTG ATTATTAGTC TGGGACCACG
  13741 ATCCCACTCG TGTTGTCGGT CTGATTATCG GTCTGGGACC ACGGTCCCAC TTGTATTGTC
  13801 GATCAGACTA TCAGCGTGAG ACTACGATTC CATCAATGCC TGTCAAGGGC AAGTATTGAC
  13861 ATGTCGTCGT AACCTGTAGA ACGGAGTAAC CTCGGTGTGC GGTTGTATGC CTGCTGTGGA
  13921 TTGCTGCTGT GTCCTGCTTA TCCACAACAT TTTGCGCACG GTTATGTGGA C
(pCAG-iCre)
SEQ ID NO: 13
          1 TTACCAATGC TTAATCAGTG AGGCACCTAT CTCAGCGATC TGTCTATTTC GTTCATCCAT
        61 AGTTGCCTGA CTCCCCGTCG TGTAGATAAC TACGATACGG GAGGGCTTAC CATCTGGCCC
      121 CAGTGCTGCA ATGATACCGC GAGACCCACG CTCACCGGCT CCAGATTTAT CAGCAATAAA
      181 CCAGCCAGCC GGAAGGGCCG AGCGCAGAAG TGGTCCTGCA ACTTTATCCG CCTCCATCCA
      241 GTCTATTAAT TGTTGCCGGG AAGCTAGAGT AAGTAGTTCG CCAGTTAATA GTTTGCGCAA
      301 CGTTGTTGCC ATTGCTACAG GCATCGTGGT GTCACGCTCG TCGTTTGGTA TGGCTTCATT
      361 CAGCTCCGGT TCCCAACGAT CAAGGCGAGT TACATGATCC CCCATGTTGT GCAAAAAAGC
      421 GGTTAGCTCC TTCGGTCCTC CGATCGTTGT CAGAAGTAAG TTGGCCGCAG TGTTATCACT
      481 CATGGTTATG GCAGCACTGC ATAATTCTCT TACTGTCATG CCATCCGTAA GATGCTTTTC
      541 TGTGACTGGT GAGTACTCAA CCAAGTCATT CTGAGAATAG TGTATGCGGC GACCGAGTTG
      601 CTCTTGCCCG GCGTCAATAC GGGATAATAC CGCGCCACAT AGCAGAACTT TAAAAGTGCT
      661 CATCATTGGA AAACGTTCTT CGGGGCGAAA ACTCTCAAGG ATCTTACCGC TGTTGAGATC
      721 CAGTTCGATG TAACCCACTC GTGCACCCAA CTGATCTTCA GCATCTTTTA CTTTCACCAG
      781 CGTTTCTGGG TGAGCAAAAA CAGGAAGGCA AAATGCCGCA AAAAAGGGAA TAAGGGCGAC
      841 ACGGAAATGT TGAATACTCA TACTCTTCCT TTTTCAATAT TATTGAAGCA TTTATCAGGG
      901 TTATTGTCTC ATGAGCGGAT ACATATTTGA ATGTATTTAG AAAAATAAAC AAATAGGGGT
      961 TCCGCGCACA TTTCCCCGAA AAGTGCCACC TGGGTCGACA TTGATTATTG ACTAGTTATT
    1021 AATAGTAATC AATTACGGGG TCATTAGTTC ATAGCCCATA TATGGAGTTC CGCGTTACAT
    1081 AACTTACGGT AAATGGCCCG CCTGGCTGAC CGCCCAACGA CCCCCGCCCA TTGACGTCAA
    1141 TAATGACGTA TGTTCCCATA GTAACGCCAA TAGGGACTTT CCATTGACGT CAATGGGTGG
    1201 AGTATTTACG GTAAACTGCC CACTTGGCAG TACATCAAGT GTATCATATG CCAAGTACGC
    1261 CCCCTATTGA CGTCAATGAC GGTAAATGGC CCGCCTGGCA TTATGCCCAG TACATGACCT
    1321 TATGGGACTT TCCTACTTGG CAGTACATCT ACGTATTAGT CATCGCTATT ACCATGGTCG
    1381 AGGTGAGCCC CACGTTCTGC TTCACTCTCC CCATCTCCCC CCCCTCCCCA CCCCCAATTT
    1441 TGTATTTATT TATTTTTTAA TTATTTTGTG CAGCGATGGG GGCGGGGGGG GGGGGGGCGC
    1501 GCGCCAGGCG GGGCGGGGCG GGGCGAGGGG CGGGGCGGGG CGAGGCGGAA AGGTGCGGCG
    1561 GCAGCCAATC AGAGCGGCGC GCTCCGAAAG TTTCCTTTTA TGGCGAGGCG GCGGCGGCGG
    1621 CGGCCCTATA AAAAGCGAAG CGCGCGGCGG GCGGGAGTCG CTGCGTTGCC TTCGCCCCGT
    1681 GCCCCGCTCC GCGCCGCCTC GCGCCGCCCG CCCCGGCTCT GACTGACCGC GTTACTCCCA
    1741 CAGGTGAGCG GGCGGGACGG CCCTTCTCCT CCGGGCTGTA ATTAGCGCTT GGTTTAATGA
    1801 CGGCTCGTTT CTTTTCTGTG GCTGCGTGAA AGCCTTAAAG GGCTCCGGGA GGGCCCTTTG
    1861 TGCGGGGGGG AGCGGCTCGG GGGGTGCGTG CGTGTGTGTG TGCGTGGGGA GCGCCGCGTG
    1921 CGGCTCCGCG CTGCCCGGCG GCTGTGAGCG CTGCGGGCGC GGCGCGGGGC TTTGTGCGCT
    1981 CCGCAGTGTG CGCGAGGGGA GCGCGGCCGG GGGCGGTGCC CCGCGGTGCG GGGGGGGCTG
    2041 CGAGGGGAAC AAAGGCTGCG TGCGGGGTGT GTGCGTGGGG GGGTGAGCAG GGGGTGTGGG
    2101 CGCGGCGGTC GGGCTGTAAC CCCCCCCTGC ACCCCCCTCC CCGAGTTGCT GAGCACGGCC
    2161 CGGCTTCGGG TGCGGGGCTC CGTACGGGGC GTGGCGCGGG GCTCGCCGTG CCGGGCGGGG
    2221 GGTGGCGGCA GGTGGGGGTG CCGGGCGGGG CGGGGCCGCC TCGGGCCGGG GAGGGCTCGG
    2281 GGGAGGGGCG CGGCGGCCCC CGGAGCGCCG GCGGCTGTCG AGGCGCGGCG AGCCGCAGCC
    2341 ATTGCCTTTT ATGGTAATCG TGCGAGAGGG CGCAGGGACT TCCTTTGTCC CAAATCTGTG
    2401 CGGAGCCGAA ATCTGGGAGG CGCCGCCGCA CCCCCTCTAG CGGGCGCGGG GCGAAGCGGT
    2461 GCGGCGCCGG CAGGAAGGAA ATGGGCGGGG AGGGCCTTCG TGCGTCGCCG CGCCGCCGTC
    2521 CCCTTCTCCC TCTCCAGCCT CGGGGCTGTC CGCGGGGGGA CGGCTGCCTT CGGGGGGGAC
    2581 GGGGCAGGGC GGGGTTCGGC TTCTGGCGTG TGACCGGCGG CTCTAGAGCC TCTGCTAACC
    2641 ATGTTCATGC CTTCTTCTTT TTCCTACAGC TCCTGGGCAA CGTGCTGGTT ATTGTGCTGT
    2701 CTCATCATTT TGGCAAAGAA TTGATTAATT CGAGCGAACG CGTGCCGCCA CCATGGTGCC
    2761 CAAGAAGAAG AGGAAAGTCT CCAACCTGCT GACTGTGCAC CAAAACCTGC CTGCCCTCCC
    2821 TGTGGATGCC ACCTCTGATG AAGTCAGGAA GAACCTGATG GACATGTTCA GGGACAGGCA
    2881 GGCCTTCTCT GAACACACCT GGAAGATGCT CCTGTCTGTG TGCAGATCCT GGGCTGCCTG
    2941 GTGCAAGCTG AACAACAGGA AATGGTTCCC TGCTGAACCT GAGGATGTGA GGGACTACCT
    3001 CCTGTACCTG CAAGCCAGAG GCCTGGCTGT GAAGACCATC CAACAGCACC TGGGCCAGCT
    3061 CAACATGCTG CACAGGAGAT CTGGCCTGCC TCGCCCTTCT GACTCCAATG CTGTGTCCCT
    3121 GGTGATGAGG AGAATCAGAA AGGAGAATGT GGATGCTGGG GAGAGAGCCA AGCAGGCCCT
    3181 GGCCTTTGAA CGCACTGACT TTGACCAAGT CAGATCCCTG ATGGAGAACT CTGACAGATG
    3241 CCAGGACATC AGGAACCTGG CCTTCCTGGG CATTGCCTAC AACACCCTGC TGCGCATTGC
    3301 CGAAATTGCC AGAATCAGAG TGAAGGACAT CTCCCGCACC GATGGTGGGA GAATGCTGAT
    3361 CCACATTGGC AGGACCAAGA CCCTGGTGTC CACAGCTGGT GTGGAGAAGG CCCTGTCCCT
    3421 GGGGGTTACC AAGCTGGTGG AGAGATGGAT CTCTGTGTCT GGTGTGGCTG ATGACCCCAA
    3481 CAACTACCTG TTCTGCCGGG TCAGAAAGAA TGGTGTGGCT GCCCCTTCTG CCACCTCCCA
    3541 ACTGTCCACC CGGGCCCTGG AAGGGATOTT TGAGGCCACC CACCGCCTGA TCTATGGTGC
    3601 CAAGGATGAC TCTGGGCAGA GATACCTGGC CTGGTCTGGC CACTCTGCCA GAGTGGGTGC
    3661 TGCCAGGGAC ATGGCCAGGG CTGGTGTGTC CATCCCTGAA ATCATGCAGG CTGGTGGCTG
    3721 GACCAATGTG AACATTGTGA TGAACTACAT CAGAAACCTG GACTCTGAGA CTGGGGCCAT
    3781 GGTGAGGCTG CTCGAGGATG GGGACTGAGC GGCCGCACTC CTCAGGTGCA GGCTGCCTAT
    3841 CAGAAGGTGG TGGCTGGTGT GGCCAATGCC CTGGCTCACA AATACCACTG AGATCTTTTT
    3901 CCCTCTGCCA AAAATTATGG GGACATCATG AAGCCCCTTG AGCATCTGAC TTCTGGCTAA
    3961 TAAAGGAAAT TTATTTTCAT TGCAATAGTG TGTTGGAATT TTTTGTGTCT CTCACTCGGA
    4021 AGGACATATG GGAGGGCAAA TCATTTAAAA CATCAGAATG AGTATTTGGT TTAGAGTTTG
    4081 GCAACATATG CCCATATGCT GGCTGCCATG AACAAAGGTT GGCTATAAAG AGGTCATCAG
    4141 TATATGAAAC AGCCCCCTGC TGTCCATTCC TTATTCCATA GAAAAGCCTT GACTTGAGGT
    4201 TAGATTTTTT TTATATTTTG TTTTGTGTTA TTTTTTTCTT TAACATCCCT AAAATTTTCC
    4261 TTACATGTTT TACTAGCCAG ATTTTTCCTC CTCTCCTGAC TACTCCCAGT CATAGCTGTC
    4321 CCTCTTCTCT TATGGAGATC CCTCGACCTG CAGCCCAAGC TTGGCGTAAT CATGGTCATA
    4381 GCTGTTTCCT GTGTGAAATT GTTATCCGCT CACAATTCCA CACAACATAC GAGCCGGAAG
    4441 CATAAAGTGT AAAGCCTGGG GTGCCTAATG AGTGAGCTAA CTCACATTAA TTGCGTTGCG
    4501 CTCACTGCCC GCTTTCCAGT CGGGAAACCT GTCGTGCCAG CGGATCCGCA TCTCAATTAG
    4561 TCAGCAACCA TAGTCCCGCC CCTAACTCCG CCCAGTTCCG CCCATTCTCC GCCCCATGGC
    4621 TGACTAATTT TTTTTATTTA TGCAGAGGCC GAGGCCGCCT CGGCCTCTGA GCTATTCCAG
    4681 AAGTAGTGAG GAGGCTTTTT TGGAGGCCTA GGCTTTTGCA AAAAGCTAAC TTGTTTATTG
    4741 CAGCTTATAA TGGTTACAAA TAAAGCAATA GCATCACAAA TTTCACAAAT AAAGCATTTT
    4801 TTTCACTGCA TTCTAGTTGT GGTTTGTCCA AACTCATCAA TGTATCTTAT CATGTCTGGA
    4861 TCCGCTGCAT TAATGAATCG GCCAACGCGC GGGGAGAGGC GGTTTGCGTA TTGGGCGCTC
    4921 TTCCGCTTCC TCGCTCACTG ACTCGCTGCG CTCGGTCGTT CGGCTGCGGC GAGCGGTATC
    4981 AGCTCACTCA AAGGCGGTAA TACGGTTATC CACAGAATCA GGGGATAACG CAGGAAAGAA
    5041 CATGTGAGCA AAAGGCCAGC AAAAGGCCAG GAACCGTAAA AAGGCCGCGT TGCTGGCGTT
    5101 TTTCCATAGG CTCCGCCCCC CTGACGAGCA TCACAAAAAT CGACGCTCAA GTCAGAGGTG
    5161 GCGAAACCCG ACAGGACTAT AAAGATACCA GGCGTTTCCC CCTGGAAGCT CCCTCGTGCG
    5221 CTCTCCTGTT CCGACCCTGC CGCTTACCGG ATACCTGTCC GCCTTTCTCC CTTCGGGAAG
    5281 CGTGGCGCTT TCTCATAGCT CACGCTGTAG GTATCTCAGT TCGGTGTAGG TCGTTCGCTC
    5341 CAAGCTGGGC TGTGTGCACG AACCCCCCGT TCAGCCCGAC CGCTGCGCCT TATCCGGTAA
    5401 CTATCGTCTT GAGTCCAACC CGGTAAGACA CGACTTATCG CCACTGGCAG CAGCCACTGG
    5461 TAACAGGATT AGCAGAGCGA GGTATGTAGG CGGTGCTACA GAGTTCTTGA AGTGGTGGCC
    5521 TAACTACGGC TACACTAGAA GAACAGTATT TGGTATCTGC GCTCTGCTGA AGCCAGTTAC
    5581 CTTCGGAAAA AGAGTTGGTA GCTCTTGATC CGGCAAACAA ACCACCGCTG GTAGCGGTGG
    5641 TTTTTTTGTT TGCAAGCAGC AGATTACGCG CAGAAAAAAA GGATCTCAAG AAGATCCTTT
    5701 GATCTTTTCT ACGGGGTCTG ACGCTCAGTG GAACGAAAAC TCACGTTAAG GGATTTTGGT
    5761 CATGAGATTA TCAAAAAGGA TCTTCACCTA GATCCTTTTA AATTAAAAAT GAAGTTTTAA
    5821 ATCAATCTAA AGTATATATG AGTAAACTTG GTCTGACAG
(SynHLA_FullClone)
SEQ ID NO: 14
          1 GGGTCCTTTT CATCACGTGC TATAAAAATA ATTATAATTT AAATTTTTTA ATATAAATAT
        61 ATAAATTAAA AATAGAAAGT AAAAAAAGAA ATTAAAGAAA AAATAGTTTT TGTTTTCCGA
      121 AGATGTAAAA GACTCTAGGG GGATCGCCAA CAAATACTAC CTTTTATCTT GCTCTTCCTG
      181 CTCTCAGGTA TTAATGCCGA ATTGTTTCAT CTTGTCTGTG TAGAAGACCA CACACGAAAA
      241 TCCTGTGATT TTACATTTTA CTTATCGTTA ATCGAATGTA TATCTATTTA ATCTGCTTTT
      301 CTTGTCTAAT AAATATATAT GTAAAGTACG CTTTTTGTTG AAATTTTTTA AACCTTTGTT
      361 TATTTTTTTT TCTTCATTCC GTAACTCTTC TACCTTCTTT ATTTACTTTC TAAAATCCAA
      421 ATACAAAACA TAAAAATAAA TAAACACAGA GTAAATTCCC AAATTATTCC ATCATTAAAA
      481 GATACGAGGC GCGTGTAAGT TACAGGCAAG CGATCCGTCC TAAGAAACCA TTATTATCAT
      541 GACATTAACC TATAAAAATA GGCGTATCAC GAGGCCCTTT CGTCTCGCGC GTTTCGGTGA
      601 TGACGGTGAA AACCTCTGAC ACATGCAGCT CCCGGAGACG GTCACAGCTT GTCTGTAAGC
      661 GGATGCCGGG AGCAGACAAG CCCGTCAGGG CGCGTCAGCG GGTGTTGGCG GGTGTCGGGG
      721 CTGGCTTAAC TATGCGGCAT CAGAGCAGAT TGTACTGAGA GTGCACCATA AATTCCCGTT
      781 TTAAGAGCTT GGTGAGCGCT AGGAGTCACT GCCAGGTATC GTTTGAACAC GGCATTAGTC
      841 AGGGAAGTCA TAACACAGTC CTTTCCCGCA ATTTTCTTTT TCTATTACTC TTGGCCTCCT
      901 CTAGTACACT CTATATTTTT TTATGCCTCG GTAATGATTT TCATTTTTTT TTTTCCACCT
      961 AGCGGATGAC TCTTTTTTTT TCTTAGCGAT TGGCATTATC ACATAATGAA TTATACATTA
    1021 TATAAAGTAA TGTGATTTCT TCGAAGAATA TACTAAAAAA TGAGCAGGCA AGATAAACGA
    1081 AGGCAAAGAT GACAGAGCAG AAAGCCCTAG TAAAGCGTAT TACAAATGAA ACCAAGATTC
    1141 AGATTGCGAT CTCTTTAAAG GGTGGTCCCC TAGCGATAGA GCACTCGATC TTCCCAGAAA
    1201 AAGAGGCAGA AGCAGTAGCA GAACAGGCCA CACAATCGCA AGTGATTAAC GTCCACACAG
    1261 GTATAGGGTT TCTGGACCAT ATGATACATG CTCTGGCCAA GCATTCCGGC TGGTCGCTAA
    1321 TCGTTGAGTG CATTGGTGAC TTACACATAG ACGACCATCA CACCACTGAA GACTGCGGGA
    1381 TTGCTCTCGG TCAAGCTTTT AAAGAGGCCC TAGGGGCCGT GCGTGGAGTA AAAAGGTTTG
    1441 GATCAGGATT TGCGCCTTTG GATGAGGCAC TTTCCAGAGC GGTGGTAGAT CTTTCGAACA
    1501 GGCCGTACGC AGTTGTCGAA CTTGGTTTGC AAAGGGAGAA AGTAGGAGAT CTCTCTTGCG
    1561 AGATGATCCC GCATTTTCTT GAAAGCTTTG CAGAGGCTAG CAGAATTACC CTCCACGTTG
    1621 ATTGTCTGCG AGGCAAGAAT GATCATCACC GTAGTGAGAG TGCGTTCAAG GCTCTTGCGG
    1681 TTGCCATAAG AGAAGCCACC TCGCCCAATG GTACCAACGA TGTTCCCTCC ACCAAAGGTG
    1741 TTCTTATGTA GTGACACCGA TTATTTAAAG CTGCAGCATA CGATATATAT ACATGTGTAT
    1801 ATATGTATAC CTATGAATGT CAGTAAGTAT GTATACGAAC AGTATGATAC TGAAGATGAC
    1861 AAGGTAATGC ATCATTCTAT ACGTGTCATT CTGAACGAGG CGCGCTTTCC TTTTTTCTTT
    1921 TTGCTTTTTC TTTTTTTTTC TCTTGAACTC GACGGATCTA TGCGGTGTGA AATACCGCAC
    1981 AGATGCGTCC TAGGGCGGCC GCATAACTTC GTATAGCATA CATTATACGA AGTTATCCGG
    2041 AACCGAGTAC AAGCCCACGG TGCGCCTCGC CACCCGCGAC GACGTCCCCA GGGCCGTCCG
    2101 CACCCTCGCC GCCGCGTTCG CCGACTACCC CGCCACGCGC CACACCGTCG ATCCGGACCG
    2161 CCACATCGAG CGCGTGACCG AGCTGCAAGA ACTCTTCCTC ACGCGCGTCG GGCTCGACAT
    2221 CGGCAAGGTG TGGGTCGCGG ACGACGGAGC CGCTGTGGCG GTCTGGACCA CGCCGGAGAG
    2281 CGTCGAAGCG GGGGCGGTGT TCGCCGAGAT CGGCCCGCGC ATGGCCGAGT TGAGCGGTTC
    2341 CCGGCTGGCC GCGCAGCAAC AGATGGAGGG CCTCCTGGCT CCGCACCGGC CCAAGGAGCC
    2401 CGCGTGGTTC CTGGCCACCG TCGGAGTCTC GCCCGACCAC CAGGGCAAGG GTCTGGGCAG
    2461 CGCCGTCGTG CTCCCCGGAG TGGAGGCGGC CGAGCGCGCC GGGGTGCCCG CCTTCCTGGA
    2521 AACCTCCGCG CCCCGCAACC TCCCCTTCTA CGAGCGGCTC GGCTTCACCG TCACCGCCGA
    2581 CGTCGAGGTG CCCGAAGGAC CGCGCACCTG GTGCATGACC CGCAAGCCCG GTGCCGCATG
    2641 CCCCACGCTA CTGCGGGTTT ATATAGACGG TCCCCACGGG ATGGGGAAAA CCACCACCAC
    2701 GCAACTGCTG GTGGCCCTGG GTTCGCGCGA CGATATCGTC TATGTACCCG AGCCGATGAC
    2761 TTACTGGCGG GTGCTGGGGG CTTCCGAGAC AATCGCGAAC ATCTACACCA CACAACACCG
    2821 CCTCGACCAG GGTGAGATAT CGGCCGGGGA CGCGGCGGTG GTAATGACAA GCGCCCAGAT
    2881 AACAATGGGT ATGCCTTATG CCGTGACCGA CGCCGTTCTG GCTCCTCATA TCGGGGGGGA
    2941 GGCTGGTAGC TCACATGCCC CGCCCCCGGC CCTCACCCTC ATCTTCGACC GCCATCCCAT
    3001 CGCCGCCCTC CTGTGCTACC CGGCCGCGCG GTACCTTATG GGCAGCATGA CCCCCCAGGC
    3061 CGTGCTGGCG TTCGTGGCCC TCATCCCGCC GACCTTGCCC GGCACCAACA TCGTGCTTGG
    3121 GGCCCTGCCG GAGGACAGAC ACATCGACCG CCTGGCCAAA CGCCAGCGCC CCGGCGAGCG
    3181 GCTGGACCTG GCTATGCTGG CTGCGATTCG CCGCGTTTAC GGGCTACTTG CCAATACGGT
    3241 GCGGTATCTG CAGTGCGGCG GGTCGTGGCG GGAGGACTGG GGACAGCTTT CGGGGACGGC
    3301 CGTGCCGCCC CAGGGTGCCG AGCCCCAGAG CAACGCGGGC CCACGACCCC ATATCGGGGA
    3361 CACGTTATTT ACCCTGTTTC GGGCCCCCGA GTTGCTGGCC CCCAACGGCG ACCTGTATAA
    3421 CGTGTTTGCC TGGGCCTTGG ACGTCTTGGC CAAACGCCTC CGTTCCATGC ACGTCTTTAT
    3481 CCTGGATTAC GACCAATCGC CCGCCGGCTG CCGGGACGCC CTGCTGCAAC TTACCTCCGG
    3541 GATGGTCCAG ACCCACGTCA CCACCCCCGG CTCCATACCG ACGATATGCG ACCTGGCGCG
    3601 CACGTTTGCC CGCGAGATGG GGGAGGCTAA CGAGCTCGGC AGTGGAGAGG GCAGAGGAAG
    3661 TCTGCTAACA TGCGGTGACG TCGAGGAGAA TCCTGGCCCA AGCCAGTTCG ACATCCTGTG
    3721 CAAGACCCCC CCCAAGGTGC TGGTGCGGCA GTTCGTGGAG AGATTCGAGA GGCCCAGCGG
    3781 CGAGAAGATC GCCAGCTGTG CCGCCGAGCT GACCTACCTG TGCTGGATGA TCACCCACAA
    3841 CGGCACCGCC ATCAAGAGGG CCACCTTCAT GAGCTACAAC ACCATCATCA GCAACAGCCT
    3901 GAGCTTCGAC ATCGTGAACA AGAGCCTGCA GTTCAAGTAC AAGACCCAGA AGGCCACCAT
    3961 CCTGGAGGCC AGCCTGAAGA AGCTGATCCC CGCCTGGGAG TTCACCATCA TCCCTTACAA
    4021 CGGCCAGAAG CACCAGAGCG ACATCACCGA CATCGTGTCC AGCCTGCAGC TGCAGTTCGA
    4081 GAGCAGCGAG GAGGCCGACA AGGGCAACAG CCACAGCAAG AAGATGCTGA AGGCCCTGCT
    4141 GTCCGAGGGC GAGAGCATCT GGGAGATCAC CGAGAAGATC CTGAACAGCT TCGAGTACAC
    4201 CAGCAGGTTC ACCAAGACCA AGACCCTGTA CCAGTTCCTG TTCCTGGCCA CATTCATCAA
    4261 CTGCGGCAGG TTCAGCGACA TCAAGAACGT GGACCCCAAG AGCTTCAAGC TGGTGCAGAA
    4321 CAAGTACCTG GGCGTGATCA TTCAGTGCCT GGTGACAGAA ACCAAGACAA GCGTGTCCAG
    4381 GCACATCTAC TTTTTCAGCG CCAGAGGCAG GATCGACCCC CTGGTGTACC TGGACGAGTT
    4441 TCTGAGGAAC AGCGAGCCCG TGCTGAAGAG AGTGAACAGG ACCGGCAACA GCAGCAGCAA
    4501 CAAGCAGGAG TACCAGCTGC TGAAGGACAA CCTGGTGCGC AGCTACAACA AGGCCCTGAA
    4561 GAAGAACGCC CCCTACCCCA TCTTCGCTAT CAAGAACGGC CCTAAGAGCC ACATCGGCAG
    4621 GCACCTGATG ACCAGCTTTC TGAGCATGAA GGGCCTGACC GAGCTGACAA ACGTGGTGGG
    4681 CAACTGGAGC GACAAGAGGG CCTCCGCCGT GGCCAGGACC ACCTACACCC ACCAGATCAC
    4741 CGCCATCCCC GACCACTACT TCGCCCTGGT GTCCAGGTAC TACGCCTACG ACCCCATCAG
    4801 CAAGGAGATG ATCGCCCTGA AGGACGAAAC CAACCCCATC GAGGAGTGGC AGCACATCGA
    4861 GCAGCTGAAG GGCAGCGCCG AGGGCAGCAT CAGATACCCC GCCTGGAACG GCATCATCAG
    4921 CCAGGAGGTG CTGGACTACC TGAGCAGCTA CATCAACAGG CGGATCACCG GTTGCGTACG
    4981 CGGATCCTCT GCTGGAGACA TGAGAGCTGC CAACCTTTGG CCAAGCCCGC TCATGATCAA
    5041 ACGCTCTAAG AAGAACAGCC TGGCCTTGTC CCTGACGGCC GACCAGATGG TCAGTGCCTT
    5101 GTTGGATGCT GAGCCCCCCA TACTCTATTC CGAGTATGAT CCTACCAGAC CCTTCAGTGA
    5161 AGCTTCGATG ATGGGCTTAC TGACCAACCT GGCAGACAGG GAGCTGGTTC ACATGATCAA
    5221 CTGGGCGAAG AGGGTGCCAG GCTTTGTGGA TTTGACCCTC CATGATCAGG TCCACCTTCT
    5281 AGAATGTGCC TGGCTAGAGA TCCTGATGAT TGGTCTCGTC TGGCGCTCCA TGGAGCACCC
    5341 AGTGAAGCTA CTGTTTGCTC CTAACTTGCT CTTGGACAGG AACCAGGGAA AATGTGTAGA
    5401 GGGCATGGTG GAGATCTTCG ACATGCTGCT GGCTACATCA TCTCGGTTCC GCATGATGAA
    5461 TCTGCAGGGA GAGGAGTTTG TGTGCCTCAA ATCTATTATT TTGCTTAATT CTGGAGTGTA
    5521 CACATTTCTG TCCAGCACCC TGAAGTCTCT GGAAGAGAAG GACCATATCC ACCGAGTCCT
    5581 GGACAAGATC ACAGACACTT TGATCCACCT GATGGCCAAG GCAGGCCTGA CCCTGCAGCA
    5641 GCAGCACCAG CGGCTGGCCC AGCTCCTCCT CATCCTCTCC CACATCAGGC ACATGAGTAA
    5701 CAAAGGCATG GAGCATCTGT ACAGCATGAA GTGCAAGAAC GTGGTGCCCC TCTATGACCT
    5761 GCTGCTGGAG GCGGCGGACG CCCACCGCCT ACATGCGCCC ACTAGCCGTG GAGGGGCATC
    5821 CGTGGAGGAG ACGGACCAAA GCCACTTGGC CACTGCGGGC TCTACTTCAT CGCATTCCTT
    5881 GCAAAAGTAT TACATCACGG GGGAGGCAGA GGGTTTCCCT GCCACAGCTG GCTCCGGCGA
    5941 GGGCAGGGGA AGTCTTCTAA CATGCGGGGA CGTGGAGGAA AATCCCGGCC CAGTGAGCAA
    6001 GGGTGAGGAG GATAACATGG CCTCTCTCCC AGCGACACAT GAGTTACACA TCTTTGGCTC
    6061 CATCAACGGT GTGGACTTTG ACATGGTGGG TCAGGGCACC GGCAATCCAA ATGATGGTTA
    6121 TGAGGAGTTA AACCTGAAGT CCACCAAGGG CGACCTCCAG TTCTCCCCCT GGATTCTGGT
    6181 CCCTCATATC GGGTATGGCT TCCATCAGTA CCTGCCCTAC CCTGACGGGA TGTCGCCTTT
    6241 CCAGGCCGCC ATGGTAGATG GCTCTGGATA CCAAGTCCAT CGCACAATGC AGTTTGAAGA
    6301 TGGTGCCTCC CTTACTGTGA ACTACCGCTA CACCTACGAG GGAAGCCACA TCAAAGGAGA
    6361 GGCCCAGGTG AAGGGGACTG GTTTCCCTGC TGACGGTCCT GTGATGACCA ACTCGCTGAC
    6421 CGCTGCGGAC TGGTGCAGGT CGAAGAAGAC TTACCCCAAC GACAAAACCA TCATCAGTAC
    6481 CTTTAAGTGG AGTTACACCA CTGGAAATGG CAAGCGCTAC CGGAGCACTG CGCGGACCAC
    6541 CTACACCTTT GCCAAGCCAA TGGCGGCTAA CTATCTGAAG AACCAGCCGA TGTACGTGTT
    6601 CCGTAAGACG GAACTCAAGC ACTCCAAGAC CGAGCTGAAC TTCAAGGAGT GGCAAAAGGC
    6661 GTTTACCGAT GTGATGGGCA TGGACGAGCT GTACAAGTGA ATTCGATCAT AATCAGCCAT
    6721 ACCACATTTG TAGAGGTTTT ACTTGCTTTA AAAAACCTCC CACACCTCCC CCTGAACCTG
    6781 AAACATAAAA TGAATGCAAT TGTTGTTGTT AACTTGTTTA TTGCAGCTTA TAATGGTTAC
    6841 AAATAAAGCA ATAGCATCAC AAATTTCACA AATAAAGCAT TTTTTTCACT GCATTCTAGT
    6901 TGTGGTTTGT CCAAACTCAT CAATGTATCT TATCATGTCT GGCTCGAGGG AAGTTCCTAT
    6961 ACTCTAGAAA GAATAGGAAC TTCAAAGGAA GTGCTTCAGG GTGAAGGAAA ATGATACTAG
    7021 ATGGTGACCC AGATATACAG AAAGGAACAA TTAACAACAG AAATGATGCA CATACACATG
    7081 TCACATTCAC ACTCATTTTC TTAATTTCCT GAAGACATAT GGCTACTTGT TTAAAACAAA
    7141 AAGTATTACA CTGTATCATT GAGCTTATAA TATAGGTTGA TGTAATATAT ACAACAATAA
    7201 TAGCACAATG GTAGGTTACA AGAAACTACA CTGTTAGAAG TGTCCTTTAT TTTCTGGATG
    7261 CAGOTTAATA TAACCTGAAC TTCACTGTGA AAAGTCAAGG AATCGGGTTT CCATTCTTTG
    7321 CTATTGTGTA AAGAAATATA GCCAAAAGCC ACTAGGAGAA TTAAAACCAT AAACTAAAAA
    7381 ATGTTTATTT GACACATAAG AAAGTAGTAA AGGAGGAACA GAAACAAAAA GATATGAGAC
    7441 AAATTGAAAA CGTATAGCAA AATTGTAGAC CAAAACCCAA CCATTGCAAG TGAAGAAATG
    7501 ACATGACCTG AGTCACATTA GCAGGACTGC TGAGCACTGT GGGGAGAACA GATATGGGCA
    7561 GGAGGTGAGG GACAGTGTTA GTGCCACAAT TCAGGAGGGA AAGGGTGGCA GGGACTAACG
    7621 GGGGTGGTGT GAAGGATGAG AAGGGCAGAC AGAAGGGCTG GAGAGGCAGG AGGAGAGGAA
    7681 AAGGAGCAGG AGAAAGAATT CTAAAGCAGT GGAAGAGCCT GGCAGAGGGT TCTTTGCATT
    7741 CGGTATTTAA TACATTTTGT TGGACTTCCT AAAAACTAAT TGGCTCCTTA TGATTAAAAA
    7801 AAAAAGAGTT ACAAAAATAC CAAGTGTTCA GATAAAATAT GCACACTGCT TGGATGTGCA
    7861 GAGTTCAGGA AAACAGGCAG TGCTTGAGCG CTGGTGAAGA GCATTGGGAC TGCATGGAGC
    7921 CCTCGCAACT TTGAGGTGAT GACTACAGGT TCCCGGTTGC AATAGACAGT AACAAACCCT
    7981 GCTTCTTTAT ATTCAGGAGA TGTTCTGGAC TCACACAGGG AAACTCAGGG TGGGGAATGA
    8041 AGATAATTTT AAATGCAACA ACCCAGAGTC ACAGATCCAT AGTCTGGGAA AGTAAAACTT
    8101 AGGAGCTTTG AGAGTTTAAT TGTAATGCTG TTTTGACACA GGTCTTTTGC AGATTGGAAT
    8161 TCTAATCATT CAGGGATTAC CAATATTGTG CTACCTACTG TATTAATAAA CAAAAAGGAA
    8221 ACTGGTCTCT ATGAGAATCT CTGTGTGGTG GCTTCAGACA AAACTTCGCC AGGTTTAGAG
    8281 AGAAAACCCC TGTCTCTACA CCTCCATTCC CAGGGCGAGC TCACTCTCTG GCATCAAGTT
    8341 CTCCGTGATC AGCTTCCCTA CACAAGANTC CAAGAGGAGA GGTAAGGAGT GAGAGGCAGG
    8401 GAGTCCAGTT CAGGGACAGG GATTCCAGGA GGAGAAGTGA AGGGGAAGCG GGTGGGCGCA
    8461 GCCTGGGGGT CTCTCCCTGG TTTCCACAGA CAGATCCTTG TGCAGGACTC AGGCAGACAG
    8521 TGTGACAAAG AGGCTGGTGT AGGAGAAGAG GGATCAGGAC GAAGTCCCAT GTCCCGGACG
    8581 GGGCTCTCAG GGTCTCAGGC TCCGAGGGCC GCGTCTGCAT TGGGGAGGCG CAGCGTTGGG
    8641 GATTCCCCAC TCCCACGAGT TTCACTTCTT CTCCCAACCT ATGTCGGGTC CTTCTTCCAG
    8701 GATACTCGTG ACGCGTCCCC ATTTCCCACT CCCATTGGGT GTCGGGTGTC TAGAGAAGCC
    8761 AATCAGTGTC GCCGGGGTCC CAGTTCTAAA GTCCCCACGC ACCCACCCGG ACTCAGAGTC
    8821 TCCTCAGACA CCGAGATGCG GGTCACGGCA CCCCGAACCC TCCTCCTGCT GCTCTGGGGG
    8881 GCCCTGGCCC TGACCGAGAC CTGGGCCGGT GAGTGCGGGT CGGGAGGGAA ATGGCCTCTG
    8941 TGGGGAGGAG CGAGGGGACC GCAGGCGGGG GCGCAGGACC CGGGGAGCCG CGCCGGGAGG
    9001 AGGGTCTGGC GGGTCTCAGC CCCTCCTCGC CCCCAGGCTC CCACTCCATG AGGTATTTCT
    9061 ACACCGCCAT GTCCCGGCCC GGCCGCGGGG AGCCCCGCTT CATCGCAGTG GGCTACGTGG
    9121 ACGACACGCA GTTCGTGAGG TTCGACAGCG ACGCCGCGAG TCCGAGAGAG GAGCCGCGGG
    9181 CGCCGTGGAT AGAGCAGGAG GGGCCGGAGT ATTGGGACCG GAACACACAG ATCTACAAGG
    9241 CCCAGGCACA GACTGACCGA GAGAGCCTGC GGAACCTGCG CGGCTACTAC AACCAGAGCG
    9301 AGGCCGGTGA GTGACCCCGG CCCGGGGCGC AGGTCACGAC TCCCCATCCC CCACGTACGG
    9361 CCCGGGTCGC CCCGAGTCTC CGGGTCCGAG ATCCGCCTCC CTGAGGCCAC GGGACCCGCC
    9421 CAGACCCTCG ACCGGCGAGA GCCCCAGGCG CGTTTACCCG GTTTCATTTT CAGTTGAGGC
    9481 CAAAATCCCC GCGGGTTGGT CGGGGCGGGG CGGGGCTCGG GGGACGGGGC TGACCGCGGG
    9541 GCCGGGGCCA GGGTCTCACA CTTGGCAGAC GATGTATGGC TGCGACCTGG GGCCGGACGG
    9601 GCGCCTCCTC CGCGGGCATA ACCAGTTAGC CTACGACGGC AAGGATTACA TCGCCCTGAA
    9661 CGAGGACCTG AGCTCCTGGA CCGCGGCGGA CACCGCGGCT CAGATCACCC AGCGCAAGTG
    9721 GGAGGCGGCC CGTGAGGCGG AGCAGCTGAG AGCCTACCTG GAGGGCACGT GCGTGGAGTG
    9781 GCTCCGCAGA TACCTGGAGA ACGGGAAGGA GACGCTGCAG CGCGCGGGTA CCAGGGGCAG
    9841 TGGGGAGCCT TCCCCATCTC CTATAGGTCG CCGGGGATGG CCTCCCACGA GAAGAGGAGG
    9901 AAAATGGGAT CAGCGCTAGA ATGTCGCCCT CCCTTGAATG GAGAATGGCA TGAGTTTTCC
    9961 TGAGTTTCCT CTGAGGGCCC CCTCTTCTCT CTAGGACAAT TAAGGGATGA CGTCTCTGAG
  10021 GAAATGGAGG GGAAGACAGT CCCTAGAATA CTGATCAGGG GTCCTCTTTG ACCCCTGCAG
  10081 CAGCCTTGGG AACCGTGACT TTTCCTCTCA GGCCTTGTTC TCTGCCTCAC ACTCAGTGTG
  10141 TTTGGGGCTC TGATTCCAGC ACTTCTGAGT CACTTTACCT CCACTCAGCT CAGGAGCAGA
  10201 AGTCCCTGTT CCCCGCTCAG AGACTCGAAC TTTCCAATGA ATAGGAGATT ATCCCAGGTG
  10261 CCTGCGTCCA GGCTGGTGTC TGGGTTCTGT GCCCCTTCCC CACCCCAGGT GTCCTGTCCA
  10321 TTCTCAGGCT GGTCACATGG GTGGTCCTAG GGTGTCCTAT GAGAGATGCA AAGCGCCTGA
  10381 ATTTTCTGAC TCTTCCCATC AGACCCCCCA AAGACACACG TGACCCACCA CCCCATCTCT
  10441 GACCATGAGG CCACCCTGAG GTGCTGGGCC CTGGGCTTCT ACCCTGCGGA GATCACACTG
  10501 ACCTGGCAGC GGGATGGCGA GGACCAAACT CAGGACACTG AGCTTGTGGA GACCAGACCA
  10561 GCAGGAGATA GAACCTTCCA GAAGTGGGCA GCTGTGGTGG TGCCTTCTGG AGAAGAGCAG
  10621 AGATACACAT GCCATGTACA GCATGAGGGG CTGCCGAAGC CCCTCACCCT GAGATGGGGT
  10681 AAGGAGGGGG ATGAGGGGTC ATATCTCTTC TCAGGGAAAG CAGGAGCCCT TCTGGAGCCC
  10741 TTCAGCAGGG TCAGGGCCCC TCGTCTTCCC CTCCTTTCCC AGAGCCATCT TCCCAGTCCA
  10801 CCATCCCCAT CGTGGGCATT GTTGCTGGCC TGGCTGTCCT AGCAGTTGTG GTCATCGGAG
  10861 CTGTGGTCGC TACTGTGATG TGTAGGAGGA AGAGCTCAGG TAGGGAAGGG GTGAGGGGTG
  10921 GGGTCTGGGT TTTCTTGTCC CACTGGGGGT TTCAAGCCCC AGGTAGAAGT GTTCCCTGCC
  10981 TCATTACTGG GAAGCAGCAT CCACACAGGG GCTAATGCAG CCTGGGACCC TGTGTGCCAG
  11041 CACTTACTCT TTTGTGCAGC ACATGTGACA ATGAAGGACG GATGTATCAC CTTGATGGTT
  11101 GTGGTGTTGG GGTCCTGATT TCAGCATTCA TGAGTCAGGG GAAGGTCCCT GCTAAGGACA
  11161 GACCTTAGGA GGGCAGTTGG TCCAGGACCC ACACTTGCTT TCCTCGTGTT TCCTGATCCT
  11221 GCCTTGGGTC TGTAGTCATA CTTCTGGAAA TTCCTTTTGG GTCCAAGACG AGGAGGTTCC
  11281 TCTAAGATCT CATGGCCCTG CTTCCTCCCA GTCCCCTCAC AGGACATTTT CTTCCCACAG
  11341 GTGGAAAAGG AGGGAGCTAC TCTCAGGCTG CGTGTAAGTG GTGGGGGTGG GAGTGTGGAG
  11401 GAGCTCACCC ACCCCATAAT TCCTCCTGTC CCACGTCTCC TGCGGGCTCT GACCAGGTCC
  11461 TGTTTTTGTT CTACTCCAGC CAGCGACAGT GCCCAGGGCT CTGATGTGTC TCTCACAGCT
  11521 TGAAAAGGTG AGATTCTTGG GGTCTAGAGT GGGCGGGGGG GGGTGGGGTG GGGAGGGGGC
  11581 AGAGGGGAAA GGCCTGGGTA ATGGAGATTC TTTGATTGGG ATGTTTCGCG TGTGTGGTGG
  11641 GCTGTTTAGA GTGTCATCAC TTACCATGAC TAACCAGAAT TTGTTCATGA CTGTTGTTTT
  11701 CTGTAGCCTG AGACAGCTGT CTTGTGAGGG ACTGAGATGC AGGATTTCTT CACTCCTCCC
  11761 CTTTGTGACT TCAAGAGCCT CTGGCATCTC TTTCTGCAAA GGCACCTGAA TGTGTCTGCG
  11821 TCCCTGTTAG CATAATGTGA GGAGGTGGAG AGACAGCCCA CCCCCGTGTC CACTGTGACC
  11881 CCTGTTCCCA TGCTGACCTG TGTTTCCTCC CCAGTCATCT TTCCTGTTCC AGAGGGGTGG
  11941 GGCTGGATGT CTCCATCTCT GTCTCAACTT TATGTGCACT GAGCTGCAAC TTCTTACTTC
  12001 CCTACTGAAA ATAAGAATCT GAATATAAAT TTGTTTTCTC AAATATTTGC TATGAGAGGT
  12061 TGATGGATTA ATTAAATAAG TCAATTCCTG GAATGTGAGA GAGCAAATAA AGACCTGAGA
  12121 ACTTTCCAGA ATCTGCATGT TCGCTGTGCT GAGTCTGTTG CAGGTGGGGT GTGGAGAAGG
  12181 CTGTGGGGGG CCGAGTGTGG ACAGGGCCTG TGCCCAGTTG TTGTTGAGCC CATCATGGGC
  12241 TCTATGTGGT TAGTCCTCAG CTGGGTCACC TTCACTGCCC CATTGTCCTT GTCCCTTCAG
  12301 CGGAAACTTG TCCAGTGGGA GCTGTGACCA CAGAGGCTCA CACATCGCCC AGGGTGGCCC
  12361 CTGCACACGG GGGTCTCTGT GCATTCTGAG ACAAATTTTC AGAGCCATTC ACCTCCTGCC
  12421 CTGCTTCTAG AGCTCCTTTT CTGCTCTGCT CTCCTGCCCT CTCTCCCTGC CCTGGTTCTA
  12481 GTGATCTTGG TGCTGAATCC AATCCCAACT CCTGAATCTG TAAAGCAGAG TCTAATTTAG
  12541 ACTTACATTT GTCTGTGAAA TTGGACCCAT CATCAAGGAC TGTTCTTTCC TGAAGGGAGA
  12601 ACCTGATTGT GTGCTGCAGT GTGCTGGGGC AGGGGGTGCG GGGAGGGGGT TGCTGTAGAA
  12661 AGAGGGATGG GGAGGGATGG CCTGAGGCTA TGTTAATAAA GATACTGCCT TCAAAATAGG
  12721 GAGGTGCTCT ACAGTGATCA TTCATTCAAC TGACCTTTGT CATTGGCCAG ACATAGGACA
  12781 GAATGGTTCT GCATCTGGGG AACACCACTG AAGTAAAATC AGAAAAATCT CTGGCCTTTT
  12841 GTAGCATATG TTCCAGTGGG AAGAGGCAGA CGATAGATAC ACTATAACCA GAGTAAGGAA
  12901 GGAAAGTGCT AGAAGGTGGT AAGTGCTGTG AGGCAGGTGA TCCAGGATGT GGGCAGTGGG
  12961 GACAGGGAAG GTGGCTGTTG TGCTGGGTAG TCAGTGTGTG CCTTGTTGCA AAGGTGACTT
  13021 TTGAGGAAAG ATTTGAGAGA CATGAGGATG TCTGGGGAAG TTCTTTCCAG GCAGAGGAAG
  13081 CTCCAGTCCA AATGCACTAT GGCAGGAAGG TGTTTGTGTT CCCAGAAGAG CAAGGAGGCC
  13141 AGGAGGGCTG GACGGAGAGA AACTGAGGTG AGGTCAGAGG TGCGGCCAGA ACAGGTGGGC
  13201 TTGAGGGGAG TGGGGTTGGA TCTGGCCTTT GCTCTGAGTG GGATGGGGAG TTAGAGGACA
  13261 GTTTTGAGCA GAAGAGAGCC ATGATATGAC TTCTGTTTTA AAAGGATCTC TCTGACGGCT
  13321 GTGCTGAGAA CAGAATTGAG AGGCAAGGGA CGAGGGAGGC AGAAGGGAAA ACAGTAGGAA
  13381 TCAAGTGCAG TATTCCAGGC TGGAGATGTC GGTTACCTTG ACTGGGGCGT GAGCAGGGGA
  13441 AATAGTGGGA CGTGAGGGGA TTCTGGATGC ATTTGAAGAT GGACTCACAG CATTTGCCAA
  13501 TGGATTGTAT CTGTGATGTG AGAAAGACGA ATCAAGGACA CCCATAGTTG TAAAATGAGT
  13561 GAGTAGAAGG ATGGAGCTGC TGTCAGTGGA GATGGGGAGA CTCTGGCAGG AGCGTCCTGA
  13621 AGAGGGGACA TCACAGGCAC TCAGTGGAGG AGATGTCTAC TAGGAGGGCG GGTGGGGGAG
  13681 CTGGGTGGAA TTTGGACAGA CAACTCCAGA GTTTAGGGGA AAGGACTGGG CTGGAGAAAT
  13741 AGATTTAGGA GGTCACACCA TATATATGAG ACTTAAAACC TCAAGCATGG ATGAGGCACA
  13801 AAGGGAGTGA CTGACTATGG AAAAGAATGA GCACAAGGAC TGAACCCTGG ACCTCCAGTT
  13861 CTAAGGGGTG TGATCAGATC ACACCCAGAG CAGACTGCAC AGTTCTGGCC CCATGTCTAG
  13921 AGGACACTCA GACAAGGAAA CCCCATGTGC ACCAGGATCA TCTGGATGTG GTGCTGAGAT
  13981 CCTGGAAGTC TGGAGTTGAG CAAGAGATTC TGGATTTATG ACAAGGCTGG AGCTCATGTT
  14041 GCTGGTCTCC AGATCACACT TGGAGTAGCA AGAACACCAG GATCCCACAC GTCTGAGCAT
  14101 CAGCCTCACC TCTAGGGCTT GTCATATAAA TGATTCCTTG GTCTTGTGCA TAATTCTCTG
  14161 AGACAAGGGT TCTGGGGAGT GGCCTGTGTA TTTTCTAAGT CCCCACCAGC AATCCTATTG
  14221 CTCAGACAGA TTGGGAGCCA CTGAGATCAG TGATCAGAGA GTGCCCAGGG TGGGTGGGTG
  14281 GGGTGGGTTT TCAAACCCTG TTGAAAAGAG GATTTTTCTC ACAGAAAGAA AAGGGAGGAT
  14341 GTATATCATC AGTTATGAGA GGTGATATTC TCTGTTGTTC TCTCCACCAT GGGGTAGAGG
  14401 CCAGGTAGAC AATTCAGGAT GTGGCTCTCG CACAAAGAAC ACCTCTGAAT GCCGCTCTCT
  14461 GACACTCGCC CGCAGACTCA TTTCTCACTC ACTTCTTGGA GAAAACTATG GAAACCAAAT
  14521 TTCTGTAATG TACACATAAA GTTGTATATT GGTATTGGGG GCTAGTTTTA TTGTGGGGAA
  14581 GGCCACAGAA CCAGGCTGGA AACTACACAT CCAAGAACAG AATTCACAGC CCACCCTGGA
  14641 TCAGGTCCCT CCTAGGAACA ACTGCCCCTG CTGCTGAGCA CAGACAGCAC TGCTCACACC
  14701 TCTGACATCC TGGTGCTGGA CACTGGACCC TGAGGCTAGG ACAGATGTCA CTGCTGCACC
  14761 TGGAAATTGG ACATCACTAC TGTCACTCAG CCATCTTTAC TAAAATGCTT TCTGCACAGT
  14821 CCTAGCCTCT CTGTCACCTC ATTCTGGCTG AGTCTGGCAG TGATATAGGA GTTAAGAAGA
  14881 AATTATTTAG ACAGGTAGTG AGGGTACGCA AGGCTTTTAA TGGAAAGCAG GCTCCAAATT
  14941 ATTTTCTTTT CTAACAAGGA GCAGCCTGTA ATATCAAGCT TCAGACATAG ACAAGTAAGC
  15001 TGGAAGTTTG CACGGGTGAA TGCCAGCAGC TGTCCAATAG GAATAGGACG CCTGGGACTG
  15061 GGCATGTTCA CTATGGCGGC TCCATCTTCC CTTCTCTTTG CCAGGCACGT TTATAGTAAG
  15121 GAGCAGACAA CATGTCACCG GCCAAGTGGA AAGCCCATTT GCATAATAGG ATGAGGGTGG
  15181 GGTGAACAGC CTTCCACACG CACTATGTAA ATATCACACC TGGTACAACC AACCTGTGGG
  15241 CCCTACATAA ATCAGACACC ATCTCCTCAA GCCTGCCTGT AAAATCCGAT GCACTCCATT
  15301 CCAGGCTGGA ATTCCCTTTT GGGTGCCCCT CTCTCTCACA AGAAGGAGCT GTTTCCCTTT
  15361 GTCTTTCTTT TGCCTATTAA ACCCTTTGAT CCTAACTCAC TCCTCGTGTG TCAGTGTCCT
  15421 TAATCTTCTT GGCGTGAGAT GACGAACACT TAGTATTTAC CTGAGACAAT GACACCTCTT
  15481 CAGCAGGTGG TCATCAGACT GGTGGAGCTT AAAATTCATG TCCATGATCA ACTGCAGGGG
  15541 TGACTGGAGT GTTGTTGTCC TCTTCCAGGG AGGGAGGTGG TGTCTGTCTC CTCTCAACAT
  15601 TTTACATGTT GTAAACCAAA AATAAACATC AAAGCCACCC CTTCCCAATC ATTTTAATGG
  15661 ACACCCTCAG CCAGGGTGCT CAAAAGTTAA CCTGAAAGAG TGGCTCAGGC CACCATGGGA
  15721 AGCAGGTATT GAACATGTCT TATTATGCCC TCTTCCCTCT TGGAATTCAG GAAAAGTTGA
  15781 CCAGCATTTA CCATCAACAC AGACCTTCAG TCTGATCAGA AACATTTACA ATCTATTCTC
  15841 TTTGAAGCCT GGCACCTGGA GGCGTCATCT GTATGAGAAT TTTGGACTCC ACAACCTTTT
  15901 ATCATAACCC AGACATTCCT TTCTATTGAT AATAACAACC AATTGCCAAT CAGAAAAATT
  15961 AAAAATCTAC TTATAACCCA GAAGCACTAC CCCGCAACCC TTGCTTGCTT CAAATTGTTC
  16021 CAACTTTCTG GACCGAACCA ATGTATAGCT TAAATGTATT TGATTGATGT CTCATATCTT
  16081 CCTTAAATGT GTAAAACCAT GTATTTGATT GATGTCTCAT ATCTTCCTTA AATGTGTAAA
  16141 ACCAACCTGT GCCTCAACCA CCTTGAGCAC ATGTTCTCGG TCTCCTGAGG GCTGTGTCAT
  16201 GGGCTGTGGT CACTCATTTT TGGCTCAGAA TAAATCTCTT TAAATATGTT ACTGCGTTTG
  16261 ACTCTTTTCA TTGACAGTGT GGAATTTTGA TGTAGTAAGC GGGTTCAAGT GCTGGAGTGT
  16321 GAGGGGTGGG GAAAAGTGAC AAATTTTAAT TCTTGGAGCA GTGATCCAGG ACGAGAACTT
  16381 TATCTAGTAC CCAGTAGGCA CGAAGAGTGG CTGAATGAAC CAGTGACTAA TAAATAACAT
  16441 ATTTCCACCC ATTCCCTTGA GGATAGGTTT ACATCAAGTT TTTGTCTATC CCAGTTTATA
  16501 GTCTAGATTA TTGGAGTGGC ATTCTGTAAT GATGAAATGA TTATTCAAAC CTCTTCCCTC
  16561 AGCATGACTC TTACTAGGAT TAAAACTCCC AGGTTTGTAA GATATCAGAG GTTCTGGTTC
  16621 CACACGATGT TTTATATTAA TATTTATATT TTCTTGTGTG TGGAAATATT TCTGGGGAGA
  16681 GAATCTCTAA CATTTATTAG TTTTTATGTG ACACCCTCAA AGGCCTAAAA AATCCTAACT
  16741 TCTAGATTAA AGGTAGGACC ACTCCACGTC TAGACAATGA ACAGCTGGTA GCAACCTGTG
  16801 TTTAGGGACA TGAAACAAAT GCTTACATTT GTTTATCTTT TCCTCTGGGA CCAAAGAAGG
  16861 TAGAGGTTTA CTTGCTTACT CTTACTATAA GTTTCTCTCT GTGGGATGGT GATGGGGTTT
  16921 TCTTCCACCC AAATAACTTT CTGATTCTCC AACAGCAGCT GGGAGTCCCA CAATTTGACT
  16981 CAATTCTGAC ACTAACTACC TGGAGTTCGC CTCAGACTCT ACAGGTTTAC CAGCTCAGTC
  17041 CTACAAGTCT GTCCTCACTT CAGAAGCAAG TCACAAGTAT CGGGTCCCAG GTTACCTGCA
  17101 CTTCTGTCTA ACATGGCTGC AAAGTCAGGG GTTTCTCACC TCCCAAGGAT TGAGAATTCT
  17161 TAAACTAACT CAAAGAACTC AGGAAGTTGC TATAGCCGTG TAAACCAAAA AACGTCTGAG
  17221 ACAGGTCTCA GTCAGTTTAG AAGTTTATTT TGCCAAGCTG AAGACGCACC TTGGAAACAG
  17281 AGACACAAAT CATTGTAGCA TCTGTGGCCC ATGCTCTTTC CTAACAGGCT TTTGAGGACT
  17341 TCAATATGTA CAGGGGAAAG AGCAGCCAGG AGAGGAAAAA AATAGTCAAC TATGCATTCA
  17401 TCTCATGCTC AGTAAATCTG CATTTTAAAT AAGATAAAGT AAACATTGGG TGGAAAAGTC
  17461 AAATATGCGT TTGACTCTGG GTGGGTGGAG GGATGATTTC TAGTCTTGTC TTTGTCCTGT
  17521 ACCTGTGAAG ATAAGCTGTT AATTTACATT GTCAGGGGAA AATTCAACAA AACTGTTTTA
  17581 GGGTAAACAC GTTGGGGCCC ACAAGGAATT ATCTTGTGAG CAGTTTGTGA GGGAGGCCAC
  17641 CTGGGGAGAT ATGTGGTCTT CTATCTTTGC AGTTATTTGT TTAGGAACGA AAGAAAGGCA
  17701 GTTTTTCCAT CAGTCTCCAA ACTTAATTTT TCCTTTTGGT ATAGTGAATT TGGAGTCCTG
  17761 AAATTTTATT TTCTTTTCAC ACTCAGTATT GCAGTTTTTT ATTGTTATAG CTTAAATCCC
  17821 CACCTTTTTG GCACCTGGGA CCGGTTTCAT GGAAGACAGT TTTTCCATGG ACTAGTGGAG
  17881 GTGGTGGAGG GCAGAGGGAA ATGGTTTCGG ATGAAACTGT TGTACCTCAG ATGATCAGGC
  17941 ATTAGCGCTT CATAAGGAGC ATGCAACCTA GATCTCTCGC ATGCAGAGTT CACAATAGGG
  18001 TTCGTGCTTC TATGAGAATC TAATTCCGCC ACTGATCTGA CAGGAGGCGG AACTCTGGTG
  18061 TCATGCTCAT GCTCTCTCAC CCACTGATCA CCTCCTGCTG TGCAGTCTGG TTCCTACCAG
  18121 GCCAGGAACC TGTACCAGTC CGCAGCCCGG GGGTTAAGGA CCCCTGGTTT AAATTATGCC
  18181 ACTAGAGCAG AACTCCTGAG ACTGGATAAC TTATAACAAA CAGAAATTTG TCTCGTTCAT
  18241 GGTGTCGAGG ATGAGAATTC CCAGATCAAG GGACTCTATC TGGTAGAAAA CAAAAGGACA
  18301 AGAGAGGGTG AGAGGTGGGG GGAAAAGGAA GTCAAACTCA CAGCCTCAGG TCCTTTTATC
  18361 ATGAGCOTTA GTCCATGAAT GAAGGCAGAG CTCTGATGGT CCAATCACCT CTCAAAAGTC
  18421 CCATTTCTTA AAAATGTTGC ATTTGGGATG AAATATCCAA CAGATGOTTT TTTGGGGAAA
  18481 CATTCAAACC ATAGCAGTTA TAAAGGATAC CAAGGAGCAG CCAGATGAAG AGGTGCACAG
  18541 GGTGAGGCCT GGAAGGGCTC TGAGCACAGG AGCCTCTGTC CCCATGGAGA TGGGGCTCAC
  18601 CATCCTCCCA GCATGTGGAC ACACAGATGT GCTCTCCGAA ACCCAATCTC CAGAGGTTTC
  18661 TATGGAAATT TCATTGTGGA GGCAAGACTA ATTAAATCAC TGGTCATTGT GTGACTGAAC
  18721 TCAACCTCGA ACCTCTCTTT CCTTCCCAGA CTTTAGGGAT GGGGTTAACA GTTCCAATCC
  18781 TCTAATCACA ACTTCGTGTT TCTGGCAACC AGCCCCCATC CTGAAGCTAT CTAGGAGCCC
  18841 CTCACCCCAT AGTCATCTCA TTGCCATATT GAACTCATGT TTATCACTGA GGAGATTGCA
  18901 AATGTTTTAG AACCTGGTTG TCAGGAAACA GGGAAAAGAC CAAATATTTT TATTGGACCA
  18961 CAGATCACAC CCTGGTTTTG ACCACATTAT CTCTTTTTAT AATATTTATT ATTTTATTTT
  19021 AGAGATAATC TOTTGCTCTG TTGCCCAGGG TAGAATGCAG TGGCACAATC ATAGCTCACT
  19081 CTAACCTCGA ATTCCTGGAC TCAAGTGATC CTTCTGCCTC AGCCTCCTGA GTAGATAGCA
  19141 TTACAGGTGC ACACCAACAT GCCCAACTAA TTTTTAAATA TTTTGTAGGA ATGGGTCTCA
  19201 CTATTCTGCC CAGTCTGGTC TCAAACTCCT GGACTTAAAC AATGTTCCTA CCATTGCCTC
  19261 CCAAACTGAT GGAATTATAA GTGTGAGCCA CTGTGCCTGG CCAATCACAG ATCTCCTACA
  19321 CCAGAAGAAT CATAACAGTG AAAAGATCCT GGCACATTAC CAGAATCCTC TTTGGTCATT
  19381 AACAATGATC CCAGTGCAAT CCATAAACAG GAATAGTTTC AGCAACATAT GGCTGCACCC
  19441 TTTCAGGCGT CTGGTAAAAT TGAGCTAAGA GACAATATCA TCTCTTGCTT ATACCTTTTT
  19501 CAAGTAGTTC ATGTAACATT GGATTTTCCT CATTACATAA CCCATTTATT CATTCATTTA
  19561 CCCTCAGCTA CTATTTCTCC TTCCCTTCAT TTATACTAAA CATTTATAAT TTTGGAAGGA
  19621 ACACTAGCTT CTGCTGCTGT GCTGGCTGAG ACTGCAGGGA CCAATAGAGT TCTAGCAAGT
  19681 GTCTCCCGTC ATCCATTCCA GTTCATAGAG GGTAGGGTTA TACAGGTAGA GAACTGGTTG
  19741 GCTATCTGAT CCCAGGCAAT ATAGCTGCAT TCAGTGTTAG CCCCAACTTT GCCAGATGCA
  19801 GTGAAGGCAC AACCTACTTC TACAGGCCCT TAGGAATTAT TACATAATGG ACATAAAATA
  19861 TATTTACAGT TTCTTGCTTA GGAATAATTC CTGTTTCTGA ACTCTATTTG CATCCCTAGT
  19921 CCTGGGACCA CTGCATCAGG TATGAGAAAA GCAAGTTGAG GTGAATTACA GTCATCATTC
  19981 CAGTGTCCTC CCACCTTGGG GAGGAATATA TGTAGATCAT ACCAGCACCT TCCCCTGATC
  20041 CACCAGGAAA AGAGAGGATA CTCTCATGTC AAGTGTGAGC ACACTCATGA AGGTGTATAA
  20101 GCCCACCCTT TGTGCTTGTT TCCCCACTCT TTCCTTTACA CACCCAATTG ACTATTTTAA
  20161 TTCTCTATGG AACTCTTCCT CTGAGGAGGA TATCTCTCTG TGACCATTGT TGGACATTTT
  20221 GAGCAGGTGC ATTTTCTGGT GTAAAGAAAT ATGACTCGGT GGTCCACGTG GTTGCAGTAC
  20281 CTCCCATTCA AGTTCTTTTG TAACACTCGA GCATTTGCAT CTGAGATAAA GCAGGGACCT
  20341 CTTTTAGAAG CCTGCCGGGG CCTCCCAATC TGGAAATAAA GAAAAACCTT CAGTTCCTTC
  20401 AAGAGAAATT CCAGGCACCT AGCCAGCCCT AAAAAGTAAG TCGGTGACCT TATAAGCAAG
  20461 AAGGAATGAT AGOTTGAAAC AATAGCCAAG GAAGTTAGAG TCACAAGATG TTTCATTCTC
  20521 TATAGAAACT AAAAATAACA TCTTAACATA TGTCCCTGAG TTGTTTTTCA GAAACATGGA
  20581 TTCCCTCCTA ATAGATCCAC TGACAGGGAG ACCTTATAAA GGGGAAACTG AGAACTGAAC
  20641 TCTGACCGCC GTTCTTTGTT CTAAATTTCT TCCTGAGGGA CCTGGAGAGA GTCAAGCCCA
  20701 CAGGCCAGGC CTGAACATTC CTCTCTACTG CCCCCAACTG TGTAAGGAAG CTGTGTAAAG
  20761 AAGCTTTGCT TCCTTATCTA TCACAAATCA GAGAATCTTT AAATCTACCT AAGACCTGTA
  20821 AGCTCCCTCA TCAGGATATT CTACCTTTTA AGGCCAAAGC AGTGTAACTT GTATGCATTG
  20881 ATTTATGATG TTGCCTGTAA CTCTGCTTTC CCGAAATTTG CCCCTGCCTT TAAAAATTCT
  20941 TGCTTGTGAG CCATCAAAAA NGTTGGGTCT TAAGCGTGAG CTGCCTGATT CTCCTTGTTT
  21001 GGCGCCCTAC ACATAAATGC CCTCCTTTGT CCCACTACAA TCTCAGTGTG GATGTTTGCT
  21061 TTTACTGCCC CAGGTGAGTG GACCCCAGTT CAGTTTGGTA ACACACCTAC CACCAGCTGA
  21121 ACAAAGCCCA CTCCAGGGTG AGGATCTATT CCTGCCAAGA CCCGTTTGCT GCTTCCTGGG
  21181 GCTACTGGTA TCAGTCTAAC TTGCCAGCTA TGTATTTTCA AGGCCCTCCC ATAAAAGAAT
  21241 CTGCCACATA GCCATATGCT ATCTCTCTTT TTTTTTGTTA AAGAAAGGAC TTCTTATGTG
  21301 TATTTTGTGC CTGTGATGTG TGGAATGCAA GAGGACTATG TCTTGATTCC ACCTATCTCT
  21361 GCATTGCTAC AGCCCCAATG TCTACTTATT TCATGGACTC AGGTGATCAC CACAAGCAAA
  21421 CCTGGATTTT TTGTTGTTGT TGTTTTTGAG ATGTGGTCTT GATATGTTTC CCTGGCTGGC
  21481 CTCAAATGTT GCTCAGGTTA AACTCTAACT CCTGGACTCA AGATATCCTT CTGCCTCAGC
  21541 CGCTCTCATA GCTGGAATTA CAAGTGCACC TGGCAAACAC TTGGAGATAA ATTCTTGTTG
  21601 ATTTCAATCA CTTTCCAAAC CTGAAAGGGA GTTATTCTGA AGGACACTGA ACAGCACTGA
  21661 GGGGACGGTG CTAAACCATT CATGAGAAAC TGACCTCGTG ATCCAACCAC CTCCCTCCCT
  21721 CCAGGCCCCA CCTATAATGC TGGGGATCAC ATCTCGACAT GAGATCTGCA CAAGAACAAC
  21781 ATCCAATCTA TATCAGGCAT CAACAGATCT TACATTAATG AACCCCTCAA ATTACCATAG
  21841 TGATTTCTGT GGGACTTGGC TCATATGGGC ATTCTTTTCA TAGGCAAGTT TTTCATTGCT
  21901 TTCCTGATTA ATTATACGGC CAGGCCATTA GTCACTCCCT GTAAGTCAGT AAAAACTAAA
  21961 ACGTAGGAGC TTTATCATTG TTCAATTCTT CCACCACTGC TAAGAAAACA CCATGCAATT
  22021 AAGTCCACAA CAGCTTTTCT GTTTTTACCT TCTTTGATCA AAGAGGTGGA CTTCCAAACA
  22081 GTATGTTGTC CATTCATCTT GCAAATGCTG TACACAAACC AACCAGCTGT TAGTGGGTCA
  22141 GTTGACTGAT ACTGCAGTTC AAATCCCAGC GCTGTCTGCT ACAGAGTTCC ATCTTGCTCA
  22201 AGAGAGGTCA GTGTTTTGTT TAATTTACAC CTTCAGTGAA TTGAATGAAG CCCACTCACA
  22261 TTATAGACAT TAATCTACTT TATTCAAAGT CTGGCTATTT ATGTGTTAAT CTCATCCAAC
  22321 AAACATCCTC ACAAATCATC CAGAATAATG CTTGACCACC TCTCCAGGCA CGATGCTTTA
  22381 CCCAAGATGA CCAATAAATT TTTTTTTATT ATTATACTTT AAGTTCTAGG GTACATGTGT
  22441 ACAACGTGCA GGTTTGTTAC ATATGTATAC ATGTGCCATG TTGGTCTGCT GCACCTGTTA
  22501 ACTCATTATT TACATTAGGT ATTTCTCCTA GTACTATCCC TCCCCACTGC CCCCACCCCA
  22561 CGACAGGCCC CGGTGTGTGA TGTTCCCCCC TGTGTCCAAG TGTTCTCATT GTTCAATTCC
  22621 CACCTATGAG TGAGAACATG CGGTGTTTGG TTTTCTGTCC TTGTGATAGT TTGAACAATG
  22681 AGAACACTTG GACACTTGTG ATAGTTTGCT CAGAATGATG GTTTCCAGCT TCATCCACGT
  22741 CCCTGCAAAG GACATGAACT CATCCTTTTT ATGGCTGCAT AGTATTCCAT GGTGTATATG
  22801 TGACACATTT TCTCCCAGCA CCTGTTGTTT CCTGACTTTT TAATGATCGC CATTCTTACT
  22861 GGTGTGAGAT GGGATACCAT TGTGGTTTTG ATTTGCATTT CTCTGATGGC CAGTGATGAT
  22921 GAGCATTTTT TCATGTGTCT GTTGGCTGCA TAAATGTCTT CTTTGGAGAA GTGTCTGTTC
  22981 ATATCCTTTG CCCACTTTTT GATGGGGTTG TTTGATTTTT TCTTGTAAAT TTGTTTGAGT
  23041 TCCTTGTAGA TTCTGGATAT TAGCCCTTTG TTAGATGGGT AGATTGTAAA TATTTTCTCC
  23101 CATTTTGTAG GTTGCCTGTT CACTGTGATG GTAGTTTCTT TTGCAGTGCA GAAGCTCTTT
  23161 AGTTTAATTA GATCCCATTT GTCAATTTTG GTTTTTGATG CCATTGCTTT TGGTGTTTTA
  23221 GTCATGAAGT CCTTGCCCAT GCCTATGGCC TGAAAGGTAT TGCCTAGGTT TTCTTCTAGG
  23281 GTTTTTATGG TTTTAGGGAT GATCAGTAAA ATTAATTATG ACATTCTTCA ATATAAGAAT
  23341 ACTCTGAGGA AATTAGCCTG TGATCTAATA AAACAAGGAG CAAACAACTG TTTCTAAAAA
  23401 TTTATAACAA ATAGTCAAAA AGCCCTCGCT AGAGATTGTT TCTCACATTC ACTCTATAAT
  23461 TCTGGGTAAA AAAAAAAGCA CGTATTTGTT GAAAACCACA GAAGAAGAGA AAATTGAAAA
  23521 AGCAGAAAGT GAAAAAAATA AGAATAATAA TAAAGATGAC AGCAGTTATT TCATTGGAAA
  23581 CAATGCAAAT GAGGAGACAG ATGAGCAACA TCTTTAGAGT CCTAAAAGGA AAATGCTGTG
  23641 AAACTAGATT CTATTCCCAG AAAATAATAC CTTTTTCAAA TATATATGTA TAGTTTTAAT
  23701 TGACAAAGTA TAATACATTC ATGAGATATT GTTTGATGTA TTGATATATG TGTACCCTAT
  23761 GTAATGATTA AATTAAGCTA ACACACCTTG AATATATCTA TCAAAAAATA AAGGGATTTT
  23821 AGGCCAGGCA TGGTGGCTCA TGTCTGTCAT CCCAACACTT TGGGAGGCCA AGGTAGGTAG
  23881 ATCACGAAGT CAGGAGTCAA AACCAGCCTG ACCAACATGG TGAAACCTCG TCTCTACTAA
  23941 AAATACAAAA ATTAGCTAGG CATGGTGGCA TGCTCCTGTA ATTCCCAGCT ACTCAGGAGG
  24001 CTGAGGCAGG AGAATCACTT GAACATGGGA GGCGGAGGTT GCAGTAGGCC AATATCACAC
  24061 CATTGCACTC CAGCCTGGGC AGTAGAGTGA GACTCTGTCT CAAAAATAAA CAAACAAACA
  24121 AACAAACAAA CAAACAAATG GATTTTAGAC ATACAGGGAG TGAAAAAATA ACCAGCAGAC
  24181 CTAAACTATA ATAAATGTTT CAGTCCTATA GGCAAAAAGA AAATTAAGGG ACTAGTTGGC
  24241 ACAGCATTTA ACGATGAAGA GACAGCTTCA TCGCCCCCTC CCACTGAGAC AGAGAACACA
  24301 GCCCCAGGGC CTGGGAATTC CTCAACCCAA TCCACCACCT TGGGCACCTG TGCACTCCTC
  24361 CTGGGTACCT GAGGTTGGAT GCAATCACCT TAGCTATCAC CTACCTGCAA ACACCACCTA
  24421 TGGGCATCCC ACCCAGCCTA TCGCATCTGC TGCTAACAAT GCACACTGCT TGGGACCCAG
  24481 AGAAGCAGCC CACGACTGCC ACTGCCAAGC TGGTTGCCCA GGAGCTGAAG TCCCCAGCAC
  24541 CTACCGAGTC AACCACTGCC ACTACTGCCA TCCAAGCAAG CCACCTGGAG GTCCAGGAAT
  24601 CAGCCCACCA GTAACCACCA ATACAAATGC CAGTATATAC CACCTTGGGG CACAAAGATA
  24661 GGAACACTCA GCACACCACT GCCACCACTG GAGCCTGAAG ACTGTCTCAT GTGGCATCCC
  24721 AGTCCCCAGC ACATTTTCAC CACAGCCTCA CTAATAACCA CACCCTAATG CACAGAAGAA
  24781 ATCACAGATA CCATTAACAC TGTCTACAGC CAAAGAAATT ATACAGAGAC CATAGTACTC
  24841 TACTATGTAC TACACCCAGA ATCAAATCCA AAGTACCCCA GGCAACCAAC ACCATAGACT
  24901 CATCTTCAGG AAAATGCTCT CCACTATGAA ACAAAATTCA AAAATAGGAA GAACTGACTG
  24961 TTATATCAGA TGCAAAAATA TTAATGTTAG GACACAGAAA CATGAAAAAG CAAAAAAGAA
  25021 AAAAAAAGCC GCAAAGGAAG ACAATAGATC TTAATCAAAA AGAAATTTTC AAGATTCTAG
  25081 ATAAAGGACT CAAAACATTT ATTTTACAGA AGTTCAGTGA GATACAAAAG AATTCTGAAA
  25141 AAAAAAATAC AAAGAAAACA TAAAAATAAT TCAGGATACA AATAAGAAGT TTACAAAAGA
  25201 TATAGATTTA AAAAAAAACA ACAGAAATTC TGGAATTAAA GAATACATGA CATGAAATTC
  25261 CAAATACATT TGAAAGCTTC AACAATATAC TAGGTCAGGC AGAAGGAAGA ATTCCAAAAC
  25321 TTGAAGATAT ACATTGTGAA ATAATTCAAT AAGACAAAAA CACACACAAA AAAAGAATGA
  25381 GCAAAGCCTT TGTGACATTT GGTACAATAT AAACAACCAA ATATTCAAAT TATTGGTATC
  25441 CTCAAGAGCG AAGAGACAAA GAACAGATTA GAAAACCTAT TTAATGAAAG AATAGATGAA
  25501 TAATTCCCAA CTCTAGCAAG TGATTTAGAC ATTCAGATAC AAAATATTCA ACAATTCTGA
  25561 GAAAAATACA ATGCAAAAAG TTCCTCTCCA TGGCATATAA TCAGACTTTC TATAGTATAA
  25621 GGTAAAATGC AAATCCTAAA AATAGCAAGA GGAAAGTGTT TAGTCACCTA TAAAGGAAAC
  25681 CCCATCAGAC TAACAGCAGA TTTCTTTTTC TTTTTTCTTT CTTTTTTTTT TTTTTGAGAC
  25741 GGAGTCTCGC TGTGCTGCCA GGCTGGAGTG CAGTGGCGTG ATCTCGGTTC ACTGCAACCT
  25801 CTGCCCCCTG GGTTCAGGTG ATTCTCCTCC CTCAGCCTCC CAAGTAGCTG GGATGACAGG
  25861 CACATGCCAC CACACACAGC TAATTTTTTT TTTTTTGTAT TTTTAGTAGA GACAGGATTT
  25921 CACCAAGTTG GCCAGGATGG TCTCAATCTC TTGACATCAT GATCCGCCCA CCTCAGCCTC
  25981 CCAAAGTGCT GGGATTACAG GTGTGAGCCA CTGTGCCCAG CTCTAACAGC AGATTTCTTN
  26041 GCAGAAATCT TACAGGCCAG AAGAGAATAG GATAGCATAA TCAAAGTCCT GAAAGAAAAG
  26101 AACTGCCAAG CCAAGAGTAA TAAACCAAAC ATAAATTATT CTTCATAATT GAAAAAGAAA
  26161 TTATCTTTCC AAGACAAGCA AATGCTGAAA GGCTTCATTA CCACTAGACA GGGCCTACAG
  26221 GAAATGCTCA AGAAAGTCCT AAACCTGGAA TTGAAAGGAC AACATTTTCA ACATTTCCAC
  26281 ATTATCACCA TGAGAACATA CACAAGTATA AAACTCACTG GTAAAGCAAT CACACAAAAG
  26341 AGGAAGAGAA AGTTCTCAGA TGGTCCTACT ACAGAAATCC ACCAAAGCAT AATGACAAGC
  26401 AATAAGAGAA AAAGACAATA ACAAAAGACA TATAAAACAA CCAGAGAACA ACAATGTGAA
  26461 AGGAACAAAG CTGTTGGGAA CAGGACCCCA AAAATCTGGC CATAAACTGG CCCCAAAACT
  26521 GGCCATAAAC AAAATCTCTG CAGCACTGTG ACATGTTAAT GATGGCCATG ACGCCCACAC
  26581 TGGAAGGTTG TGGGTTTACC AGAATGAGGG CAAGGAACAC CTGGCCCACT CAGGGCAGAA
  26641 AACTGCTTCA AGTTGTTCTT CAACCACCAA CAATAGCATG AGCGATCTGT GCCTTAAAGA
  26701 CATGCTCCTG CTGCAGATAA CTAGCCAAAC CTATCCTTTT ATTTTGGCCC ATCCCTTTGT
  26761 TTCCCATAAG GAATACTTTT AGTTAATCTA TAATCTATAG AAACAATGCT TATCACTGGC
  26821 TTGAGTTAAT AAATACGGGG GTAAATCTCT ATTCGAGGCT GTCAGCTCTG AAGGCTGGGA
  26881 GACCCCTGAT TTCCCACTTC ACACCTGTAT ATTTCTGTGT GTGTGTCTTT AATTTCTCTA
  26941 GCGCCGCTGG GTTAGGGTCT CCCCAGCTGA GTTGGTCTCA GCACAAAGCC TCACATATTA
  27001 ATAAAAATCT GGAATGTAAA CAGATTAAAT TCTCCACTTA TAAGATGTAG AATGGCTGAA
  27061 TGGGTAGAAA ACATGATCTA ACTACATTCT GOTTACAAAA AAACTCACTG GTAATACCAC
  27121 CTCACCACAA TCATAGAGAC AGAAGGTAAA AGAATGGCAA CAGGTTATCT ATCGAAACCA
  27181 AAAGTGAGCA GGAGTAGCTA TACTTATAGC AGATAAAATA AACTGTAAGT CAAGAACAAT
  27241 TTTTTTAAAA GATAAGGAAG GCCATCACAT AATCATAAAG AGATCCATCC AAGAAGAGAA
  27301 TAGAACAATT CTAAATATAT ATGCACTAAA CATTGGAGCA NCTAGATTCA TAAAACAAAT
  27361 ATTACTACAT CTAACAAGAG AGATAGACTC CAATACAATA ATAGTGGGGG ACTTCAACAT
  27421 CCCAACCTCA GCATAAGACA GATCACCTAC ACAGAAAATC AAGAAAGAAA CATTGGATTT
  27481 AAACTGAATT TTACACCAAA TAAAGCTAAC AGATATTTAC AGAGCATTCT GTCTAACACT
  27541 ACAGAATATA CACTCTTTTC ATGAACACAT GGAACATTTT CCAGGAGAGA GCCTATGTTA
  27601 AGCCACAAAA CAAGTCTCAA CAAATTTTCA AAAATTGAAA TCATATTAAA TATCTTCTCT
  27661 GGCCACAATG GAATAAAACT AGAAATATAC ACCAAGAGAA ATGTTGGCAA CAAGACCAAT
  27721 ACATGGAAAT TAAACAACCT GCTCCTGAAT AACCATTGAG TCAACAGAGA AATTAAGATG
  27781 TAAATCAAAA AGTTTCTTGA AATAAATGAA AATGGAAACA CAACATACCA AAACCTGTGC
  27841 GATACAGCTA AACAGGGCTA AAAGGGAGGT TTATAGCAAT AAATACCCAC CTAAGAAAAG
  27901 TAGAAACATT ACAAATTAAC AATATAACAA TGAACCTCAT GGAACCAGAA AAGCAAGAAA
  27961 AAAAATATAC AAAATTAGCA GAAAAGAAAT AATAAAGATC ATAGTAGAAA TAAAGACTTT
  28021 TAAAAACCAT GTAAATGATC AACAAAGTGA AAAGTTTGTT CTTCAAAAAC ATAAACAAAA
  28081 TTGATAAACC ACTAGCTAAA CAAACCCAGA AAAAAAGAGA GAAAATTTAT TAAGAATAAA
  28141 AGTTCCTCTT CAAAGTTTTC CTTTTTGGTT TAGAATGATA AGTCTTAAGA AGATAGTTCC
  28201 TCTTAAAAGC TTCCTTTAAG TTTCCCTCGC TCTTCATACT AACAAATCTT CTATTTTACT
  28261 AATGACTCTT TATTGTGCCC CAAAGGAGTT GTTACATACA GTAAANAAAT AAACACATTC
  28321 TATGTTCTTG TACTTGAACC AAGGCCTAAC TCCTTTGTTC CCTAGTCTGC CTGGATCTGC
  28381 CTGGACACGC CCAGACATGT CACAGOTTGC AGCCTATGCT CTTTCCTTAT TTGGAAATAT
  28441 TATTGTCTTC CTGGTTTCCC ATAAGCGACC CCCTCCTTTC CTTGGTTCCC CATTGCGCCT
  28501 TTACCTTATT TGGGAAAGTT TAAGTTTTTA GCCAACTGGG ATCAGTTTAG ATTGTGTGGT
  28561 CCAGTTCCAG CCAATGGAGA CAGGACACAA TAGTAGAAAC AAATTGCGTT AGGAATAAAA
  28621 ACCCCTGCTT TCCTTTGTTC TGGTTGCTCT CGTGGCAATC AGGCTTACAG GTAGCAGCCT
  28681 TCTGCAGAAG TAAAAATTGC CTTGCTGAGA AATCCTTTGA TCAAGTGTTC ATTTCCTTTG
  28741 TGACTCCAAG CTTTATTTCT AACTATTTTA TGGGGGCTCT TCCAGGATTC CCATTCTCCT
  28801 ATGGGAAGGG GTCTTTGGTC ACCCCATCTA GGGGAGGTGT ACCCCACTGC AGTGGCCCCA
  28861 GGGACGGGAG ATTGAGACTA CCCATTGTGA TGAATAAATT CAGGCTCTCA GCAATGTGGA
  28921 AGGAGAAGGC CCACAAGTAC CACGATGACC AGCTAACTGT GCAGAGACCA AGGTAAAAAA
  28981 AGTCACAAGG GCAACAGTAT TTCCTTGGTG GTCGGGATAT TCTGGAGGTT AAAGGGTGTG
  29041 AAAGGTAACA AGCACTACTG CTGTGCAGAG TGAGTGAGTT CAATCTGTGG TTCCGTGGTC
  29101 ACCTCATGTG GCTTATGATG GCCCTCTTGT AGGGATCCTG ACATTGGGGT TCATACAGTC
  29161 CTGCTGATGC TAAGGGGAGC CTAATTTCCT CTGGGAAAGC AGCCAGAGTG GATGAAGCAA
  29221 AAGGAGAAGA TTGCAAGAAA CCTCCAGGGT GGGGGTTAAA CCTCCAGGGA AGGATAGGCA
  29281 AGAAATCTCT AGTTCGAGGG ACTGAGCCTA ACCAGTATTC AACATGGGAA ATACCCTGAG
  29341 TAAGACAGAA GGTAAAAAGG AAAAGGCAAA CAGTGAAATC ACCTCTGATA GTCCTTTAGG
  29401 ACTCATGTTA AAATATTGGA AGGATACAAG AGGACTAAAC ATAAGAAAAA GCAACAGATG
  29461 ATAAAGTATT ATTGTTTCAT TTGGACCAAG GAACCTATTC TAAAGCCTTC AGTTTTCTGG
  29521 CCAAAATTCG GGTCAAATGA GGCTTGGACT TGCCAACTTT TAATAGAATG TGTAAATGAT
  29581 AAGAGTCCTG TTTCCCAGGA GGAAATACAT TATGCTCTGT GTTGGCAGCA AGGGCCCATC
  29641 CTCCTTTGTC ATCTAAAAAC CAAAGGAGAC AAGCCAGAAA CTACTTCTCA TGAAATTAAT
  29701 ACCCCTTACT CCAAGAACCC CACCAACACG TGGGACGCCT TAGACCATCT TCCCCCAACA
  29761 AAACTTCTTC CCCCCACCCA GTAGACCAAC ATCCCCCACC TCCCCCTTCA GTAGAAATAA
  29821 CAGTCCCAGA CCTTTCCCCT ACTCAGACAT TTTTTCCCTC CTTTTAACCC TGATTCTTGG
  29881 GGTCATCCCC AGCTTGAATG CCCTCCCTCA GGAAGACTTC AACGTGAGTT AGAACAATGT
  29941 AAAAATGATA TCCAAAACTT CCCTTTTCCC CCTTCCTCTA GGGGTTTGGC TACAAACCTC
  30001 TTCCCACTGA GGGAAGTGCC TCTAGGAGGA GCAGGCATTG GCTTTTTAAA TGCTCCCCTG
  30061 ACCAGTTCGG AAGTCCAGAG CTTGAAGAGG GAACTCAAGC CACTCTTAGA TGATCCCTAT
  30121 GGAGTGGCAG ATCAAATTGA TCAATTTTTA GCACCCCAGT TGTATACTGT GGCTGAGTTT
  30181 ATGTCCATCA TAGGCATCCT TTTTTCAGGG GAAGAAAGGA GCATGATGCA TAGGGCCGCT
  30241 ATGACCATTT GGGAGTGTGA GCATCCTCCC GGTCAAAACG TCCCTGCAGC CAAACAGAAA
  30301 TTTCCTGCTC AAGATCCCCA GTGGGATAAC AACAACATAG CTCATAGGAG GAATATGAGA
  30361 GACCTGCAAG AAATGATAAT AAAAGGAATT AGATAATCAG TACCCTGCAC CCAGAATATG
  30421 ACCAAAGCTT TTAATATACA GCAAGGAAAA GAAGAGGGGC CTATGGAATA TTTTAATAGG
  30481 CTTAAGGAAC AAATGAGGAA ATATGOTGGT TTAGATGGAG GAGACCCACT TGGACAAGGA
  30541 ATGTTAAAAG CTTCACTTTG TCACAAATAG CTGGCCAGAC GTTACCAAGA AATTACAAAA
  30601 GATAGAAAAT TGAAAGGACT GTCCCATAAA GGAACTCTTA AGGGAGGCCC AAAAGGTGTA
  30661 TGTACGAAGG GATGAAGAAA AGCAAAAGCA CAAGGCAAAG ATTATGCTGT TOTTAAAGCA
  30721 GGGAACTCCC CAATAGACAG CTCAAAGAAA TATCGCCGGT AAATCTTTTA GGTACCCGAC
  30781 TGCCAGACTC TATAATGGAG GTAAAGGAAT CAAACCAGGG AACAAGGGAA TAAAAAGAGG
  30841 AATAGGGCAG AATAAATGCT TCAAATGTGG GAAGGAAGGT CACTTTAAAG AGAATGCCCT
  30901 GAATGGAAAA AAGAAAAAGA AATCACCCAC TTATGATCTT TGAGGAGGAA TGGGGGGCCA
  30961 GGGGCTCTAC TCTTTTTACC TCGAGTCCCA CCAAGACTCC TTGATAAATT TAGAGGTGGG
  31021 ACCCAGACCT TTTTAATTGA CTCAGGAGCT GCTGATTCCT CAGTTTGTTA TCTTCCCCCT
  31081 GGTGTAACTT GGTCACAAGA AGAACTTTTT ATTTTGGGAG CAAAAGGGGA GGGGTTTAAA
  31141 GCAAAAGTCT TAGAAGAAAC AAAAATTAAA TATCAGAACC ACTCAGTAAA TATTAAACTT
  31201 CTGTTAATCC TGGAGGCAGG AACAAACCTA TTAGGAAGAG ATTTAATGCT AAAATTAAAC
  31261 CTAGGCCTTT GTGTTAATCA AGGAAATCTC CTCCCCTCCT TAAATTTGCT TGCTACTCTA
  31321 AATGAGGGAT ACATCCATCC AGATGTATGG TCAAAGGAAG GAAACTGAGG AAAGTTACAA
  31381 ATTTCCCCAA TCCAAGTTAA ATTGAAAAAC CTGGGGCAAG AGGTAAAAAG AAAACAATAT
  31441 CCAATTCCCT TAGAAGCTAA GATCAATTTA AAGCCCACAA TAGAAAGCCT TCTCCATGGT
  31501 GGACTCATTG AACCCTGTAT GTCTCCTTAT AACACTCCCA TACTGCCTGT GAAAAAGCCA
  31561 GAGGGGTCAT ATCGGCTAGT GCAAGATCTT CGATCTATTA ATCAAATAGT TCAAACAACT
  31621 CACTCTGTCG ATCCTAATCC TTATGCTATT ATTAGTAAAA TTCCTTACAA CCATGAGTGG
  31681 TTCACAGTAA TAGATCTAAA GGATGCCTTC TGGGCTTGCC CACTAGCAAA GATAGACTTT
  31741 GAATGGGAGG ACCCTCACTC TGGTTGAAAA TAACAGTATC GATGGACAGT TTTGCCCCAG
  31801 GGATTTACAG AATCTCCAAA TTTGTTTGGT CAAATACTAG AACAGATCAT AGAAAAGTTT
  31861 TACAAACCAC CATATATATG CCTGCTCCAA TACATGGATA ATATCCTTAT TTCAGGGGAG
  31921 GATAGACAAA AAGTAGAAGG ATTTTCAATA GGTTTTTTAA AAATAATTTG CAACTGGAGG
  31981 GGTTATGAGT GTCAAAAAAG CAAACTTCGG TTTGTAGAAC CTGAAGTCAA GTATTTAGGG
  32041 GACTTAATCA GTAAGGGCAA ATGGAGAATT GGATTTGAAT GGATTGAAGG CATCATATCC
  32101 TTCATATGAT ATCCTGAGAC AAAGAAGGAA CTTAGAAAGT TTTTAGGGTT AGTAGGGTAT
  32161 TGTTGCTTAT AGAGAGACTC TTATGCTTTA GTAACCAAAC CTCTACATAA AAAAAACTTA
  32221 CACAGGATGA ATTGGACCCC CTTATATGGT TTTACAAGAA ATACAGCAGA TAAAAAAATT
  32281 AAAAGAGTTA CTAGTGACAG CCCATGTTCT AGCCTTACCC TCTTTAGAAT TGCCCTTTCA
  32341 TCTTTTTGTT AATGTAGGTA ATGAAGTAGC CTTAGGGGTA CTTACTCAAA CACACGGAGG
  32401 CCATTGGCAA CCCATAGCCT TCTTGTCAAA AATTCTTCAC CCAGTAGCCC ATGGATGGCC
  32461 CGAGTGTGTC CCATCAGTGG CAGCAACAGC CTTGCTCATA GAGGAGAGCA GAAAATTAAC
  32521 CTTTGGGGAA ATCTTATCAT TAGTACTCCT CAACAAGTGA GAACAATTCT CAATCAGAAG
  32581 GCAGGAAGGT GGCTTATTGA TTCAAGAATT TTAAAGTATG AAGCCATCTT GTTAGAAAGA
  32641 GATGATCTAA TOTTAACCAC TGATGACTCA ATTAATCCAG CTGCTTTCTT AACAGGAAAC
  32701 CCCAACCCAC AGACCCCTGA TCTTTGCCCA GAGCACAGAT GTTTAGATTT AATTAGTTAT
  32761 CAAACAGAAG TTAGACCTGA TCTAAATGAA ACACCCTTTC ATACTGGGAA ACACCTGTTT
  32821 GTAGATGGTT CCTCCCGAGT AATCAAAGGA AAAAGGCATA ACGAGTATTC AACGATAGAT
  32881 GGAGATACCC TCACAGAAAT AGAGTCTGAA AGGTTACCAA ATGACTGGTC TGCACAAACA
  32941 TGTGAGTTGT TCACATTAAA TCAGGCCTTA AAATTTCTGC AAATCAAGAA GCAACAATCT
  33001 ATACAGACTC CAGGTATGCC TTTGGAGTAG TCCATACTTT TGAAAAAATT TGGACTGAGC
  33061 GGGGCCTCAT TAACAGCAAA GGTCAAATTT TAGACCATAG GGACCTAATA ATACAAGTAC
  33121 TAGAAAATTT ACAATTGCCA AAAGAAATAG CTGTTGTACA TGTCCCACGA CATCAAAGGA
  33181 ATCCTTTATT TGAAGGCCAG GGAAATAATC TCACTGACCA AATAGCCAAG CAAGCTGCCT
  33241 CTTCTCCAGC AGAACCCATT TTTCGACTAA CCCCTTGTCT TCCATCTTCA GCTGCACTCC
  33301 CTATCTTCTC TCAAGCAGAT CAGGAAAAAC TGAAAAAAAT AGGAGCTGAA GAAAACTCAA
  33361 AAGGAAAGTG GGTATTACCA GATGGAAGGG AAATGTTACC CAAACTTTAT GAGGGAAATA
  33421 TTGTCACAGC TTCATCAAGG ATGTCACTGG GGTCCTCAAA CCATGTGTGA TGCAGTCCTT
  33481 AGGGTTTATG GGTGCATAAG AATATACACC CTCGCCAAGC AAATGGTGGA AAGTTATATA
  33541 CTGTGCAGAA AATCTAATAA GCAGACCCTC AAAAGACAAC CTCCCGGAGG GAGAAATCCT
  33601 GGGTTAAGGC GATTTCAAAG TGTCCAAGTT GACTCTACTG AAATGCCCCC AGTAGGCCGC
  33661 CTTAAGTATT TACCGGTAAT AGTAGATCAT CTTACCCACT GGGTAGAAGC CATCCCCTTT
  33721 CCAAGTTCAA CAGCCAGTAA TGTGGTCAAA GCTCTGTTAG AACATATCAT ACCCAGGTTT
  33781 GGAATAATAG AGAACATTGA TTCAGATAAT GGGACCCAAT TTACCGTGTA CATTATTAAA
  33841 AGACTAATCC AAGTATTAGG GATAAAATGG GAATATCATA CTTCCTGGCC TCCACCTTCA
  33901 TCTGGGAGAG TAGAAAGAAT GAATCAAACT TTAAAGAGTC ATCTAACCAA ATTAATTTTA
  33961 GAAACTCGCC TACCATGGAC AAAATCTCTT CCCATTGCTT TACTAAGAAT CCAAACTTCT
  34021 CCTCGGAGGG ATCTTGGCTT GTCTCCTTAT GAAATGCTTT ATGGGTTACC TTATCTAAAC
  34081 ACTACTACTG ACCTTCCTAT GATCGAAACA AAAGATCAGT TCCTTAGAAA TTATGTATTT
  34141 GGTCTGTCTT CCACTCTTTC GTCCCTCAGG ACTCAAGGTC TGGTAGCACA GACTCCACCC
  34201 CTTGAATTCT GGGCCCACCA ACATCAACCC GGAGATCACA CGTCCTTATC AAAGGCTGAA
  34261 AAGAGGGAAA GCTTGAGCCT ACCTGGGAAG GACCCTATCT GGTGCTCACA ACAACTGAAA
  34321 CAGTAGTTCG GACCACTGAG AAAGGGTGGA CCCATTATAC TCGAGTCAAG TAGGCGTCAC
  34381 CTTCTCCAGA GACATGGACC ATTATTCCGT CAACTCCCAC CAGAGTAACG ATAAAAAGAA
  34441 AAACTTAATC TATCTCTTTT TCTTCTTTTC TTTCCCTTAA CTGCCCCCAT CTCATCATTA
  34501 ATGTAACTAG ATCAAGTTTG CCCAAAATTA TTACCTTTGA TGCTTCTCTA GTCATGCCTT
  34561 GTGGAAATTT ACAGGACCAA AGACAGOTTA CTGCCTCAGA TAAATATCTT TGTCCCTCTT
  34621 GAACTTCCTC AGATTGGAAA GATATAACAA ATTCTTGTAA AGATCATGAT AACCCCAAAA
  34681 ATAGTAGCTG TATGAACTGG TGGGAATGGG ACTCTTGTCC CCTTAAAACA GAGTCCCTAT
  34741 GCTATACTTG GTCTAATGTC CTATGGAACA CCAAAGGTCC AGGCTGGACT GCTCCCACCA
  34801 GTCTTTGCCA GTCCTTAAAG CCATATATCC ATTTTACAAA AGGGATTGCC TCTTCTAATT
  34861 GTCCATATAA CCAGTGTAAT CCTATTCGGC TTACTATTAC TATTGCAACA TCCCAAAACT
  34921 CTTTCCCTTC ATTAAGTCAT ATTTATAGCA TAGGGGCCAA CATCTTGGGA AAGGATCCTA
  34981 GGGGAATTTT TGAAATCTGC TTTATTGCCC CTTCCACCTC TCTAACACAT CTTCCTCAAA
  35041 CCATCACCCC ACCTATACCC AGTGACAAAA CCAAAGTAGC TATAGTAGAA GTAAAAGACC
  35101 TGAGGTAAAC TCTAGCCACT GAAACAGGGT CTCAGGATGC AAATGCCTGG CTGGAATGGA
  35161 TTAAATATTC CATCCACACT CAAAATAAGA GCGATTGTTA TGCTTGGGCA CAGGGCAGAC
  35221 CAGAGGCCCA AACTGTCCCC TTTCCACTCA GATGGTCTTG TAATAGACCA GATATGGACT
  35281 GCATGGTAGC TCTGCTCCAA GACCTCACAG CCTGGGGTAA TAAATCGTGT CAAAGACTTT
  35341 CTTTGCTGTT CCCTGAGATC AGCCCCCAAA GGCCATTCAG CCTCCATCTT CTGGGACCAA
  35401 ATTTGCTTTG TGTCTCTCAC AACAGGGAGA AAACTTGGCT TTCCTTGGAA ACCTGACAGG
  35461 ATGCAGTGAG GTCAAGCACT TCCAAGAGCT AACCCATCAG TCCTCCCTTA TCCATCCCCG
  35521 AGCAGATGTA TGGTGGTATT GTGGTGGACC TTTGTGGGAG ACTCTGCCAA ATAACTGGAG
  35581 TGGTACTTGC ACTCTAATTC AATTGGCTAT CTCTTTCACC CTGACATTTC ATTCACAAAA
  35641 ATATTAACAA CTAGGCATCG TAGTACAAGA ACTGTTCCTG TGAGTCTTTT GATCCCAACG
  35701 TTTATATAGA TGCTATTGGA GTACCAGATG AATTTAAAGC TTGAAACCAA ATAGCTGCAG
  35761 AATTTAAGTC ATTATTCAGG TGGGTGACTA TCAATAAAAA TATAGATTGG ATAGCCAGGC
  35821 GCAGTGGCTC ACGTCTGTAA TCGCAGCACT TTGGGAGGCC AAGGCGGGCA GATCACGAGG
  35881 TCAGGAGTTT GAGACCAGCC TGACCAACAT AGTGAAACCC TGTTTCTACT AAAAATACAA
  35941 AAATTAGCTG AGTGTGGTGG CACGCACCTG TAATCCCAGC TACTCAGGAG AGTCACTTCA
  36001 ACCTGGGAGT TGGAGGTTGC AGTGAGCCGA GATCACACCA CTGCACTCCA GCCTGGGCGA
  36061 CAGACTGAGA CTCCATCTCG AAAAAAAAAA TGTAGGTTGG GTAAATTATA TTTACTACAA
  36121 TCAGCAATGA TTCATTAATT GCACAAGGGA TGCTATGAGG GAATAGCTGA GCAACTAGGA
  36181 CCCACCAGTC AGATGACTTG GGAAAATAGA ACAGCACTAG ACATGATACT AGCAGAAAAA
  36241 GGCAAGGTTT GTGTTATGAT TGGAACTCAA TGTTGTACTT TTATCCTTAA TAATACTGCC
  36301 CCTGATCGAA CTATAATAAA AGCACTACAA GGTCTTACTG CTTCATCAAA TGAACTAGCT
  36361 AAAAACTCTG GAATAAATGA TCCTTTCACT AGCTTAATGG AAAGGTGGTT TGCTAAGTGG
  36421 AAAGGACTCA TGTCCTCAAT TCTCACCTCA CTGGCTCTCA TAATCAGTGT ACTTATCCTT
  36481 GTAGAATGTT GTATTATATC TTGCATTCAT GGATTAGTGC AAAGGCTTAT AGAAAAGGCT
  36541 CTTACCAAAA CCCCTTTCAA TTCTCCTCAC CCTTACTCAG ATAAGTTGTT CTTTCTAACT
  36601 GATTAAGAAG AGCAACAAAA TCAAGATATG TTAAGGAGAT TTGAAGAGGA AGAACTGTAA
  36661 AATCAAGAGG GGGAAACTGT AAAGAATAAA AGTACCTCTC CAAAGTTTTC CTTTTTGGTT
  36721 AAGAATGATA AGTGTTAAGA AGATAGTTCC TCTTAAAATC TTTCTTTAAG TTTCCCTTAC
  36781 TCTTCATACT AACAAATCTT CTAATTTACT AATGATTCTT TATTGTGCTC CAAGTAGTTG
  36841 TTACATACAG TAAAGAAATA AACACATTCT ATGTTCTTGT ACTTGAACCA AGGCCTAACT
  36901 CCTAGTCTGC CTGGATCTGA CCAGACATGC CCAGACATGT CCCAGCTTGC AGCCTATGCT
  36961 CTTGCCTTAT TTGGAAATCT TATTGTCTTC CTGGTTTCCC ATAAGTGACC CCCTCCTTTC
  37021 CTTTGTTCCC CATTGCACCT TTACCTTATT TAGGAAAGTT AAAGTTTTTA GCCAACCGGG
  37081 ATCAGTTTAG ACTGTGCGGT CCAGCTCCAG CCAATGGACA CAGGACACAA TAGTAGAAAC
  37141 AACTTGCGTT AGGAATAAAA ACCCCTGCTT TCCCTTGTTC TGGGTGCTCT CGTGGCAATC
  37201 AGGCTTACAG GTAGCACCCT TCTGCAGAAG TAAAAATTGC CTTGCTGAGA AATACTTTGT
  37261 TCAAGTGCTC ATTTTCTTTG TGACTTCGAG CTTTATTTCT AACAGACTCA AACAAAATCA
  37321 GAAAAGAAAA GAGAAACATT ACAACTGATA TTGCAAAAGT ACAAAAGACC ATCAGAGACT
  37381 ATTATGAACA ATTGTGTACT GACAAACTGG AAAACCTGGA GGAAATGGAT AAATTCCTGG
  37441 AAACAAACAA CCTGCCAAGA TTACATTAGG AAGAATAGAA AACCTGAAGC CCTAACATAC
  37501 ATGAACCATT CTCACAGTGG TCCCAGTGGG CCCCTGACCT CTCTGTGATT ATTTCCTCAG
  37561 TGCTTGTTTG CATAATTGAA ATAGACCTAC TCAGCAACAG CCAAAATCCA CAGATCAGTT
  37621 CCTTTTTTTA ACTTTTATTT TCAGTTCAGG GGTACAAGTG CAGGCTGTTT TACAGGTAAA
  37681 CTTGTGTTGT GGGAGTTTGG TACACAGATT ATTTAATCAC CCAGGTTTTA AGCCTATTAC
  37741 CCATTAGTTA TTTTCCCTGA TCCTCTTCTT CCTCCCACCC TCCACCTTCT GAAAGGCCCC
  37801 AGTGCCCAGT GTGTGCTATT CCCCTCTATG TCTCTATGCA TTCTCATCAT TTAGCTCCCA
  37861 CATATAAGTG AGAACATGCA GTATTTGGTT TTCTGTTTCT GCATTAGTTT GCTAAGGATA
  37921 ATGGCTTCCA GCTCCACCCA TGTCCCTGCA AAGGACATGA TCACATTCCT TTTTCTGGCT
  37981 GCATAGTGTT CCATGGTATA CATGTATCAC GTTTTCTTTA TCCAGTCTAT AATTGGTGGG
  38041 CATTTAAGTT GATTCCATGT CTTTGCTATT GTGAATAGTG CTGCAATGAA CATATGTGTG
  38101 CCTATGTCTT TATAATAGAA TAATTTATAT TCCTTTGGGT AGAATGGTAT TTCTCTCTTT
  38161 AGGTCTTCAA GAAATCACCA CACTGTCTTC CACAATGGCT GACCTAATTT GCACTTCCAC
  38221 CAAGAGTATA TAAGTATTCC TTTTTCTCCA CAACTTTGCC AGCATCTGTT ATTTTTTGAC
  38281 TTTTTAATAA TAGCCATTCT GACTGGTGTG AAATGGCATC TCACTGTGGT TTAGATTTGC
  38341 ATTTCTCTAA TCATCAGTGA TGTTGAGCAT TTTTTCATGT TTGTTGGCTG CATGTATGCC
  38401 TTCTTTTGAG AAGTGTCTGT TTATATGCTT TGCCCACTTT TTAATGGGGT TGTTTTGTTT
  38461 TTTCTTGTAA ATTTGTTTAA ATTCCTTATA GATATTAGAC TTTTGTCAGA TGCATAGTTT
  38521 GCAAAAAATT TTCCCCCATT CTGTAGGTTG TTTACTCTGT CGATAGTTTC TTTTGCTGTG
  38581 CAGAGCTCTT TAGTCGAATT AGATCCCATT TGACAATTTT TGCTTGTGTT GCACTTGCTT
  38641 TTGGCATCTT TGTCATGAAA TCGTTGCCCA TACCTATGTC CTAAATGGTA TTGCCTAGGT
  38701 TCTGTTACGG TTTTTACAGT TTTGGATCTT ACATTTAAGT CTTTAATCCA TTTTGAGGTG
  38761 ATTTTGGTAT ATGGTGTAAG AAAGGGGTTC CATTTTAATC TTCTGCATAT GGCTAGCCAG
  38821 TTATCCCAGC ACAATTTATG AATAGGGAAT CCTTTCCCCA TTGCTTGTTT TTGTCAGGTT
  38881 TGTCAATGAT CATAGTTTGT GTGGTCTTAT TTCTGGGTTC TCTATTCTGT TCCATTGGTC
  38941 TATATGTCTA TTTTTGTACC AGTACCATGC TGTTTCGGTT ACTGTAGCCT TGAAGTATAG
  39001 TTGAAATCAG GTAGCATACA CTGGACGTGG TGGCTCATGC CTGTAATCCC AGCACTTTGG
  39061 GAGGCAGAGG CAGGTGGATC ATGAGGTCAG GAGTTCGAGA CCAGTCTGGT CAACAAGGTG
  39121 AAACCTTGTC TCTACTAAAG ATACAAAAAA TTAGCTGAGC GTGGTGGCAT GTGCCTGTAA
  39181 TCCCAGCTAC TCAGGAGGCT GAGAAAAGGA GAATCACTTG AACCCAGGAG GCAGAGGTTG
  39241 CAGTGAGCCA GGATTACACA ATTGCACCCC AGCCTGGATG ACAGGGTGAG ACTCCATCTC
  39301 AAAAAAAAAA AAAAAAAATC AGGTAGTGTA ATGCCTCCAG CTTTGTTCCT TTTGTTTAGG
  39361 ATTGCCTTGG CTATTCAGGC TCTTTTTTGG TTCCAGGTGA ATTTTAAAAT AGTTTTTTCT
  39421 AGTTCTGTGA AGAATGTCAG TGGTAGTGGC CGGGCACGGT GGCTCACGCC TGTAATCCCA
  39481 GCACTTTGGG AGGCTGAGGC AGTGGATCAT GAGGTCAGGA GATGGAGACC ATCCTGGCTA
  39541 ACGGGGTGAA ACCCCATCTT TACTAAAAGT ACAAAAACTT AGCCCAGCAT GGTGGCGGGT
  39601 ACCTATAGTC CCAGCTACTC GGGAGGCTGA GGTAGGAGAA TAAAAAAAAC CTCCTGAATT
  39661 CGTTGATCTT TTGAGTGGTT TTTCATGTCT CTGTCTCCTT CAGGAGACAA TACCTTTGTT
  39721 AATTTTTTAT TATTACGTCT TGTCTTCTGC TAGCTTAGGG ATATGTTTGC TCTTTGTTCT
  39781 CAAGTTCTTT TGGTTGTGAT TTTAGGTTGT TAACTTGAAA TCTTTCTAAC TTTTTGATGT
  39841 GGGCATTTAA TGCTATAAAT TTCTCTCTTA ACACTGCCTT AGCTCTTTCC CAGAGATTCT
  39901 GGTACATTGT ATCTTTGTTC TAATTATCTT CAAAGAACTT CTTGATTTGT GCCTTAATTT
  39961 CATTATTTAC CCAAAAGTCG TTCAAGAACA GGTTATTCAA TTTCTATGTA ATTGTATGGT
  40021 TTGAGTGAAT TTCTTAGTCT TTATTTTGAA TTTGATTGCA CTGTGGTCCA AGTGACTATT
  40081 TGTTATGATT TCAGTTCTTT TGCATTTGCT GAGGAGTGTT TTACTTCCAA TTGTGTGATT
  40141 GATTTTAGAG TAAGTGCCAT GTGACAATGC AAAAAATGTA TATTCCATTG CTTTGGGGTG
  40201 AAGACTTCTG TAGATATTTA TCAGGTCCAT TTGATGCAGG GCTGAGTTCA GGTCCTGAAT
  40261 ATCTTCGTTA ATTTTCTGTC TTGATAATCT GTCTAATATT GTCAGTGGAG TATTAAAATC
  40321 TCTTACTATT ATTGTGTGAG ATTCTAAGTC TCTTTGAAGG TCTCTAGGAA CCTACTTTAT
  40381 GAATGTGGGT GTTCCTGTAT AGGATGCATA TATATTTCAG ATAGTTAGAT CTCCTTGTTG
  40441 AATTGAACCC TTTACCATTA TGTAATGCCC TTCTTTGTCT TTTTTTTATC TTTTTTGGTT
  40501 TAACACCTGT TTTGTCAGAA ACAAGAGTTG CAACCCTGGC TTTTTTTCTG CTTTCCATTT
  40561 GCTTGGTAAA TTTTCTTCCA TCTTTTTGTT TTGAGCCTAT GTGTGTCATT GCATGTGAAA
  40621 TGGGTGTATT GAAGACAGCA TACTGATAGG TOTTGGTTCT TTATCCAGCT TGCCACTCTA
  40681 TGTCTTTTAA ATGGGGCATT TAGCCCATTT ACATTTAAGG TTAGTATTGA TATGTATGGA
  40741 TTTGATCCTA TCATCATGAT GTTAGCTACT TACTTTGCAG ACTTTCTTAT GTGGTTGCTT
  40801 TATTGTGTCA CTAGTCTGTG TACTTCATTG TGTTTTTGTA GTGGCTGGTA ACTGTCTTTC
  40861 CATGTTTAGT GCTTCCTTCA GGAGTTCTTG TAATGCAGGT CTGGTAGTAA TGAATTCCCT
  40921 TGGCATTTGT TTGCCTGAAA AGAATCTGAT TTCTCCTTTG CTTATGAAGC TTAGTTTGTT
  40981 TGGATATGAA ATTCTGGGTT GGAAACTCTT TTCTTTGAGA ATGTTGAATA CTGGACCTCA
  41041 GTCTCTTCTG GCTTGTAGGA TTTCCACTGA GACATCCACT GTTAGTCTGA TGGGCTTCCC
  41101 TTTGTAGGTG ATCTGGCCTT TCTCTCTGGA TACCCTTGAC ATTTTTTCTT TCATTTTAAC
  41161 CTTGGAGCAT CTGATGATTA TTTGTCTTGG GGATGATCTT CTCATGGAGC AGCTTACTGG
  41221 GGTCCTCTGC ATTTCTGAAT TTGAATGTTG GCCTGTCTAG CTAGGTTGGG GAAGTTCTCA
  41281 TGGATCATAT CCTGAAATGT TTTCCAAATT GGTTCCATTC TCCCCATCTC TTTCAGGTAC
  41341 ACCAATCATT CATACATTTG TTTCTTTACA TAATCCCATG TTTTTCAGAT GTTTTGTTCA
  41401 TTCCTTTTCA TTCTTTTTTC TCTAGTCTTG TCTTCTTGTC TTATTTCAGA AAGCCAGTCT
  41461 TCAAGCACTG AGATTCTTTT CTCCACTTGC TCTATTCTGC TATTAATACT TGTGAGTGCA
  41521 CCACGAAATT CTTATAAAGT GTTTTTTACC TCTATCAGGT TGGTTATCAG ATTCTTTTGC
  41581 TGATTCCTTC TCATCTCTAT GGGCTTATCT ACCCTCAATC TTTGAGGTTG CTGACCTTTG
  41641 AGTGGATTTT TTTTCTCTTC TTTTGTTTTC TCTTTTAACA GTCTGGCCAC TTTTCTATAG
  41701 GGCTGTACAG TTTGCTAGGG GCCTGCTACA GTCCCTAGTT GCCTCAGATT TTCCAGTACC
  41761 TGAAGGTATC ACCAGTGAAG GCTATGTAAC AGCAAAGATG GCAGTCTGCC CCTTCCTCTG
  41821 GGAGGTCTGT TCCAGGAAAG TACAGACTTG TTGCCAGCCT GAACACACCT GTAGGAGGTG
  41881 CTGGAGACCC TGGTTGGGAG GTCTTGCCCA GTCAGGAGGA ACAGGATCAG GATCTAGAGG
  41941 TTTACTAAAG GAGGAATGCT CCCACAGGAG ACACAGAAAT GAACCATTGA GCTGGAAGCT
  42001 AAGACAGCTA CCCAGGCCCT TCAGAGCCTC ATGATTCTGA ATCAACAAAG GAAGGAGTTA
  42061 CTGCACTGTC TGGGGTGATT GGTCCTGGCC ATCAGGGGGA ATTGGTCTAC TACTACACAA
  42121 TGGCAGTAAA TAGGACTGTG TCTGGAACAC AGGAGATCTT TTAGAGCATC TOTTAGTAAT
  42181 ACTCTGCCGT ATAATTAAAG TCAATAAAAA CGGCAACAAC CCAAGTCAGG TAGGACTAGT
  42241 AATGACACAT ACCCTTCAGG AATGAAGGTT TGAGTCACCC AAGCAGGAAA AGAAACCTGA
  42301 TCAGCTGACG GACTTGCTGA AAGAAATGGG AATATGGGAT GGTTAGTGAA AAACAACAAA
  42361 AAAAGTATTT ATAAATACTA GTTGTGGCCG GCACAGTGGC TTGTGCCTGT AATCCCAGCA
  42421 CTTTGGGAGG CGGAGGCAGG TGGATTGCTT AAGTCTAGGA ATGTGAGACC AACCTGAGCA
  42481 AAATGCTGAA ACTCCATCTC TACTGAAAAT AAATACAAAA AATTAGTCAG ACTTGGTGGT
  42541 GTGCTTGTAG TCCCAGCTAC TTAGGAGGTT GAGGTAGGAG GATCACTTGT GCCCAGGAGG
  42601 TGGAGGTTGT AGTGAGCCAA GGTAATGCCA CTGCACTCCA GCCTGGGCAA CAGAACAAGA
  42661 CCCTGTCTCA AATAAATAAG TAANTAANTA AGTAAATAAA TAAATAAATA ACCAGCTGTA
  42721 ACCATGTGAG CACTTACAGA AATAAAGACA GTAAATGTTA TCAGCATTAC TTCTTTATGT
  42781 TTTTATAATG ATATTTATGT CTGTGTGTAT GTATCCTTAC ATGTATTGAG CAAATACCTT
  42841 TCTTTTCATC TTTTCCTGAT GATCTGTAAT AAAACATAAG ATGTGCTAAC AATAATTCCC
  42901 TTTTATTAAT AAAAGTTAAC TTTTATTACG CATGGTATTT AGGTTACAGT ATATCAAAGA
  42961 GAAGAGTGCA TATCATGCAT CTTTTTCTGT GGAAAGGGTT AGTGTGTTTC TAGTTGGATG
  43021 CAAGTTAATT GTATCATCTT TGGCAAAAGT AAGACTTTAT AATTGTTTTT ATTTGGAGAC
  43081 GAAGTAAGGT TTAAGGAAAA TGTTAATGGG TGCCAAGTTC ACAAGCATGG ACTGGGATGG
  43141 TGGGTCTTAT GTGTCAATTT TACTAAACCA TAGCACCTAG TTATTTAATC AAACACTAAA
  43201 TTTATATGTT GATATGATGG TGTTTTGCAG TTGTGATTAA AATCTGATAT CAGTTGATTT
  43261 CATGAAAAGG AAATTACCCT AAAACATGTG GGTGGGACTT TTTGAATCAC TTGAAGGCCT
  43321 TTGGGCGAAA TGTGAGTCTT CCCAGAGAAG AAAAAATTCT GCCTCAAGAT GACAGTAGCA
  43381 ACTACACTTT AGGTTTCCAA TCTGCCAGCC TGGTCTACAA ATTTTAGATT GAAGGCTGCA
  43441 ACATTATGAC TTTCCAGCCT TCCAGCCTGC CGAGGTGTAA CTTGTGACAT CAATAACGTA
  43501 AAATGAGGGG TGGGAAAAGA TGTAAAGAAG CAGAGTTTCA TATTGATTTC AGTTCAAGAT
  43561 TGTTATAACT GTATTGTTAT ATATAATACC ACAGTAATCA CAAAGAAATA TACATAAAAT
  43621 ATACACAAAA GAAAATGAGA AGGCAGTTAA AACATATCAC TAGAAAAAAA TCGACTAAAT
  43681 GAAAAATGAC TAAAAACAAA AGAAGGTTGG AATGAAGGAG ATGAAGGACA AAACAGCTAT
  43741 AAGACATGCA TAAAACCAAC ATCACAATTA CAGAAGTAAA TCCTTCCTTA TCAGTAATTA
  43801 CTTTAAATGT AAATGTAAAT GGATTAAACT TCCCAATCAG AATTCAGAGA TTATCACAAT
  43861 GGACAGAATA AAAAGGATCT GGCTGGGCAC GGTGGCTCAC ACTTTTAATC TCAGCACTTT
  43921 GGGAGACGGT GGCTGGCAGA TCACAAGGTC AGGAGTTCGA GACCAGCCTG GCCAACATGA
  43981 TGAAACCCCG TCTCTACTAA AAATAGAAAA AAATTAGCCG GGTGTAGTGG CAGGTGCCTG
  44041 TAATCTCAGT TACAAGGGAG GCTAAGGCAG GAGAATTGCT GGAACCGGGG AGGCAGAGGT
  44101 TTCAGTGAGC TAAGATCGTA CCACTGCACT CCAGCCTGGG CAACAGAGTG AGACTCCGTC
  44161 TCAAAAAAAA AAAAAAAAGA TTCTCACTTT GGATCTAACG ACACACAGAA GTTGAAATTG
  44221 GAAAGACAGA GAAGGATATT CACAAAAATA GTAACTAAGA ATTTTTAATG ATCTCTTATA
  44281 GGGTTCTATG TGTTGGCTGG GCTCAGATGA TGGCCTGGGA GTGTTACATG GTCCAGGGAT
  44341 TGAAGTGGAA GGAACCAGAC GCAGGCAGGA TGGTTAACAG CAGTGCCCAG ACTGACACAG
  44401 CCACACTTCT GCCATATTTT ATTGGGTAAC GCAGATATCA GATCTGCTTA TGTTTAAGGA
  44461 GTGGAGAGAA CTTACCTCTT GATGAGGGAG TGGTGGGTCA AACTAAAGAA AAGCAAGTGC
  44521 GATGGAAGAT ATTTAGATAT TTTTGCAGCC CTCTTTGGAA AATACATGCA ATTCAAAACA
  44581 TCCTATCTGC CAGAAAGGAA GAACACCATT ACGCCTTACC CCTCCAGTTT AAGCCCTGTG
  44641 ATTCTATAGT ATCCTGGGGA AGGATCATGT CTTAGGAAGC AGGTGGGGAG TCGAAGCTCT
  44701 AAATAAAGCC CCATCTACAC CCACAATTTC ACTTTATTTT GATGAAATTT TGTGTCATTT
  44761 TTGATGAAAT GGGGGCTCAC AAATCTCTCT TCAGGGATCT CTCCAGTGGC GGGGACGATA
  44821 TGCTCCCTTC TCCACAGTTC TACTTTATAG ATAAGTGGAA CTAGATCTCA GTTTCAGTTA
  44881 TTTAAAACAC CAGAGACTTG GATGCATGCA TGTCACAAAA AGGTGAAAGA ATGCTCTCTG
  44941 TCACTTTCCC TTCTCTCACT CACTGCGCCC TGAACAAGAA CCTGCTCTAC CCTGACCTTT
  45001 TGAATGAGGA GGAGGGTCGC ACTCCTGCCT AACCTTCAAC TTTCCAGAGA TAAACCTCCT
  45061 GCCCAAACAG CATGGTTCAT TATAAAGACC AAACCCTACA CCAAGAGCTC TCCCCTTATC
  45121 CACCATGGCA GAAACTGAAT CCTATGGAAA GGCCCAGAAA ATTCTGGCTC GAAATGACCA
  45181 GCGTCATGTC CCTTCCACCA AAATAAAGCC AACATATTCA TCACAACATG GGGTATTGAT
  45241 AAGGATATCA AAATGATTAC CAAAAAAATT TGACAAATAT GTTCTTTTTA TTACATCAAC
  45301 TTTTGTTTCC CTCCAAATAT AAAGAAAATA AGCCTGCACA GCAGGAGAAG TTGTTCCCCT
  45361 GGGAGAGCAT CCTCTTTCCA GCTGAAAATC TGGTACTGGA ATCACTAGAA TGGAACAAGG
  45421 GTTTCTGGTA TGACACAGCA CCAGGTCTTG GGGCAGCCAG AGTTAGGGAG GGCTCACCCT
  45481 CTGGGTGACA CCAACCTTTG GTTCTCCATT TTTTTGTTCT CATTGCAAGA ACAAAATTTC
  45541 AGAGTATCAG AGCCAAAGAA ATTTACTCTA TTTTATTCTT TAGAAAATGC AATCTCAAGG
  45601 ATAATCAGAC TGTTATGGGC TAAATTGTAT CCCCCCAAAA TTGGCATGTT GATGTCCTAA
  45661 CCCCCAGTAC CTGAGAGAGT GACTGCATTT GGAGAGGTTT TAAGGAGGTC ATTAAGGTAA
  45721 AATGAGGTCA TGTGAGTGTG CCCTAATTCA ATGTGACTAG TGTCTTCATA AAAAGAGAAG
  45781 ATTAGACACA GACAGATACG GAGGAATGAC CATGTGAAGA CAGAGAGAGA ACACATCTAC
  45841 AAGCCAAGGA AAGAACCTTA GAAGAAATCA GCACTGCCAA CACCATGATC TTGAACTTCC
  45901 AGCCTCCAGA ACTGTGAGAC AATGAATTTC TGTTGTTTAA GTCACCCAGT GTGTGGTACT
  45961 TTGTTATGGC AGCTCTAGCA AATTAAAACA AACATATTAT ATTGCCCAAT GGAGAGTTTT
  46021 ACCTGACTCC ACTCTGGCTC CAATTTGATT AAGGTCTATA TGCTCTGGGA CCTCTCCAGC
  46081 TCCTCATGAG AAATGACAAA GAATGCCATG TCCAGCTCCT GGGGTCCCTG ATGGCAGTGA
  46141 GAGGCAGCTC CTAATCGAGG GAGCTTTGGG GTGACGAAGG CCTCATAGAC TTCATCACTG
  46201 TTGGGCCACT TGGCTTCAGT CCCCTGACCA CAATGGATCC ATGGGCCTGG ATTCACCTCT
  46261 GACATTCGTG TCTTTTTTTT CCACAAGTGG TCTCTGGTAG AGATGGAGGG AAAGGACACA
  46321 GGGATTCAAA ATCCTGACTA TCCTAGTGTC CTCCCCTCAC TTCCACTCAG AGACAACACG
  46381 GCCATTTCTC TAAACTCCTG AGGACGTGGC CAACATAAGT AATGGAGCAC GGGGAGGTGC
  46441 CTTGAACAGA GCTGCATGGA AGGGTGGCTG GTGGCCTCTA GACCCTTTGT CTATACGTGC
  46501 TGGGCTTCTT TAGTATGAAA TGTCCCAGAG ACAGCCACAT TCTATGCTTG GATCCTCAAA
  46561 CCAAAATGTG TGAGGGTCTT AGCCCTGGCC AACTGAGCAC TGAATTCAAG AGAGACCAGG
  46621 AAACCTTCTG AATATTGGGA CACTTTATTC CTGAATCCTG AATCCTGGAA GTTGGCTTCT
  46681 GGCCAGGGAG GCAGGTGTCA TTATATCTGG GCTGCCTCTG TGAGGCCTCT TTTCCCTCTG
  46741 CCTTTCACAT CTTTCTACCC CTGTTAAAAT CCCAGAACAG ATAGAAACAT TCTGCCTTCT
  46801 TAGATGCCTC TTGCTACAAA TTTCAATGCC ATTTGATTGA ATTAATAAAT GAAAGCAACT
  46861 TTAATACAAG ATGAGGTTTA AAGAATGGTA TTTGACATGG GTTTAGAATG AATAAATGGA
  46921 TCAAGTAATG AATTTTGAGA AAATTCATGT AAAATTTATT TTTAACCATT TTATTAACTT
  46981 GACTGTGGAA TTAATAAATG ATTCCAAATT AATACAAAAA AGTGCAATCA CAAATGTATG
  47041 AATGCCCAGA AGTGTGAATG AGGGGCAATG CACACAAGAA AAAAGGTGTG CACAGGAAGA
  47101 AATTCTTCAT TCCACGTTTT TACTGATCCC TCTGGTCAAG ATGGGCTAAA AGTCCAAAAA
  47161 TCTGCTTTCC TCACATGGTC AAATGATTCA GCATAAAATA CAACTGACTG AACACAATTA
  47221 TCAATGCTTT AAATATATTA GAAAAAATCA ATAAGGAAAA ATAATCCTAC CATCACTAAG
  47281 ATTTTAAGTT ATAATAAAAT AAAGCCTAGA AACGTATCGT TTGTTCCCAA TTCCAATGAT
  47341 TATAGGATTG CAGTGTTTGG CTTTCAAAAG ACCATTTAAA ACAACTCAGG GAAGTTTTCT
  47401 TAACAAACTC TATAGCAGGA CCACAGCTAA ACCTATGAGT AGTAGTAGAA GTAAACAATG
  47461 ACACTGTCTT TAAAATTTCA TTTTGAAAAA CATGTTTGAA AGTGGCTGAC TGGCTCCTAA
  47521 TTTAACCACT ACCATTTGAA GTCATATGTT AGATTGGCAA AGTCGTCGTA ATTTTTATGT
  47581 ATTTTATTTT TGTAATGCAA GTGCTTCACT CTGCTATCTG ATAAAAGAAA ATACCAAGCC
  47641 AGGCATGGTG ATGCATGCCT TGTAGTCCCA GCTACTCAAG AGCCTGAGGC AGGAGGTTCC
  47701 TTCAGCCCAG GAGTTCAAGG CTGCAGCAAG CTATGATGGT GCCACTGCAC TCCAGCCTGG
  47761 GCAACAGATA ATGCAATTTT AATGTAAAAT TTTAGTGTTA GGAGTTCAAC CTTGAACTGA
  47821 TTCTGTGAGA TCCTATTTGA GAGAAGCTGT TGTGTACAAT GAAGTTTGGG GAATATCAAC
  47881 AAGAAGTAAA TGCCTACCTT GTTTCTAATT TACAAACTAA AAGGAAATTC CATAAACTTT
  47941 TAATGCTAAG CTAAATCATT GTAGAATGAA TGACAAAAAA ATTATCCTAC AAAACATAAC
  48001 TAGTTGCTAG ATATATTTGA ACATTAATTA GAAAAAAGAT GAATGAAAGC CATGTTTTAG
  48061 GAATAAAAAC TACAAAAGAA CTGTAAAAGC TTATCATGCC CTTGGCTGGA GTTACGCAAA
  48121 GTTACTACTC ACCCGTAAAT TGGGATTAGC ATGGTGGCTA CTTCTTAAAG TTGATACAAG
  48181 GATTAAATGT GTTTTAATGC ATCAAAAGCA TCTAGAAAAG TGCCTGAGAC ATAGTAAGTG
  48241 CTAAAGTCTC TCTAAGTAAA TAAAAAATAT ATAAAAGTGA TGCTAAATTC ACGATTCTGA
  48301 TCCTCTGTGC ACACCCAATG TGTTTAAAAT TCTGTTCATC TTCCCTTCCA CATTCACAGT
  48361 GAAATGTTTC TGATTAATTT AATGGGCTGT GTGAGGTGTT CATTATCTTT TACATTAAAC
  48421 TTGGTACATT GTACAACACT CTTATATTGT GATAATTTAC CAAGCTAACA CAGGCTTACC
  48481 TTGTTTTTAA TCCTTTTCAC ACACAGCATA GAATTATATT GCTATATTTT TAAATTTCAG
  48541 ATGATTTGAA AAATATTTAC ACTATAGTAG CCAAACAGTA ATAATTATAA GCAACTTGGA
  48601 TAAAACATTT GAAATTAGAG AACTATATTT AGAATAAATT TCTCACCTTA CAGAAAAATT
  48661 TCCCATTGAA AACCCCTTTA TTTCGCATAC AAATAAGAAT TCAGCATGTT TTTCTATACC
  48721 CAGAGAGAAC AGAGATAAAC AGGGTCCTTT CGTGTGGTTT GGGTATTTTA TTAGATGCTG
  48781 AGTTCAAGAA AGTCTCACCC CTGAAGAAGC TGAAATTCCA TTTTTCGAAG CTTGCTCCTC
  48841 AATTCCACAT GCTTATCATA GATATCAAGC AGTGCCCTTG CTGTCTGCCC ATCTTTTCTG
  48901 ACCCTGGGAC ACCACCCTTT ATTTACTTTC CCCACAACTC CTTGAAGGTC AAGCCCCCTT
  48961 GGCTGATACT CTGGGTTATC ACAGTAAATG TGCCCTCTAT CTTTTGCAGG TCACTGGAAA
  49021 GGAGGGTTCC TAAAGCATTC ACTCCTGACC CTGAGGGAGG TGGGTGCACT CACTCCAGGA
  49081 CTCAGGTTCA CCACAGATAA GCAAAAAAGT CCTCACATTC AAAATTGTCC TGGGCCACAT
  49141 GCAGCCCACG AGCCGCGGGT TGGACAAGCC TGCATCTAGA ACGTATGCAC CATAACCCCA
  49201 GACATTTTTC TGTTTTTACT TTAAGGATGC TTTCTAAATG CAAAAGCAGT CATATCCCTA
  49261 GCAGACAACA AATGTCAGTT GAATGAATGA TCACTGTAGA GCACCTCCCT ATTCTAAAGC
  49321 CCATATCTTT ATTAATGTAG CCACAGGGCA AATGCTGTTT TGTGGCAGCT AAAGAACATA
  49381 AGTTCACGTG GACATCCATG CCGCCAGTGC TTTTCTCACC AGGGCTGCTT GTGTGTCCTC
  49441 CCTCCTCCCA CACCAACCCT CCTGCACACT TCAGCACAAA ACTATATATT TCTCTTCAGG
  49501 AACAATAACC CTAGCCTTAT GGGTACAATT TTCCAACCAC ATATGAATCT AAATTAGACT
  49561 CTGCTTTATA AATCTATGAG TTTGGATTGG AGCCAGCACT AGGATTACTA CAACTCAGAG
  49621 CAGGAAGAAG AGTAGGAGGG CAGAAGAGGA GTTCCAACAG AAAGTTAACC ATGATGAGAA
  49681 ACTATGGGAC CCCTCCTCTC TGCAAATGTC ACAATCTGGT TCCTTTAAAA AGATTGGAGA
  49741 AAAATCGAAA ATACCATTAA GGAAAAAGCC CTGGGAGGAA GGCAAGACCT GGACAGGTCT
  49801 GAAAATTTGT CTCTTGATAC CACAAGGAGA TTTGTATGCA GGGACCACCC TAGGTGACAT
  49861 CCAACTCCCT GTGATCACAG GTCTTGGTGG GACAAGGTTC TACTGAAGGG CCAAGGACAA
  49921 TGGAGCAGCA AAGATGACCC AGGTGAGCTG TGACCACATA AAGCCCATGG TGGCCAGAAC
  49981 AGAAACTGGG CACAGCCCCA TCTACTCTCC TCCCCTGCAA CAAATCAGCA CAAGAAACAC
  50041 GTGGACTCTG GAAGGTTCTC ATGTCTTCCA TTTATTTTGT CTCTCAAATT TTAGGAATCT
  50101 TCTCCTTTAA TTAACTCATC AACCTCTCAT GGCAAGAATT TGAGAAAGTA AATTTATACT
  50161 CAGGTTCTAA TTTTAATAGG GAAGGAAGAA GTTACAGCTC AGTGCACCAT GAAGTTGAGA
  50221 CAGAGATGGA GACACCTCAG CCCCACCTCT CTGGAACAGG AAAGATGATT GGGGAGGGAG
  50281 CACAGGTCAG CGTGGGAAGA GGGTCATGGT GGACATGGGG GTGGGGTGGT CTCCCCACCT
  50341 CCTCACATTA TGCCTACACG AACACAGACA CATGCAGGTG CCTTTGCAGA AACAAAGTCA
  50401 GGGTTCTTCA AGTCACAAAG GGAAGGGCAG GAACAACTCT TGCCTCTCAG TCCCACACAA
  50461 GGCAGCTGTC TCACACTATA GAAAAAAATA TTCATGAACA AATTCATATC CATCACAGTG
  50521 AGGGGTCACA CCTTAAACAG CCCATCGCAT GCTCAATACA TCCAATGCAA AGAAACCCCA
  50581 TAGCACAGCT GTGTCCACTG TTCCGCCCAA CACCCAACAC ACATTAGGTC CTCCAAGCTC
  50641 TCACCTTTAC AAGCTGTGAG AGACACATCA GAGCCCTGGG CACTGTCACT GCCTGGGGTA
  50701 GAACAAAAAC AGAACCTGGT CAGATCCCAC AGAAGATGTG GCTAGAGGAG GAATTGTGGG
  50761 GTGGGTGAGC TCCCCCATGG GCTCCCAAAC ACAATATCCC AAGGACCTCA GGCATCAGCC
  50821 TCCTTCATAC TTACTTGCAG CCTGAGTGTA ACTCCCTCCT TTTCTATCTG TGAGAAGAAA
  50881 ATGTCCTGTG AGATACCAGA AAGGAGCCAG GGCCTTAAGG TCCTAGAGGA ACCTCCTAGT
  50941 CTTGGACCCC AGAGAAGTTT CCAGAAATGT GTGACTGCAG ACCCAGGGCG GGATCAGGAA
  51001 ACATGAAGAA AGCAGGTGTG GGTCCTGGAC CAATAGCCCT CCTGAGGTCT GTCCTCAGGG
  51061 ACCTTCCCCT GTGACTTGTG ACTGCTGGGA TCAGGTCCCA TCACCGCCGT AATCAAGGTG
  51121 ATAAATCTGT CCTTCATTTT AACAGGTGCT TTACAAAAGA GTAAGTGCTG GCACACAGGG
  51181 CCCAGGCTGG GTAGGCCCAT GATTGTGGAT GGTGCTTCCC AGTAATGAGA CAGGGCACAT
  51241 TTCTAGCTGG GGCTTGGAAC CCTCAGTGAG ACAAGAAATC TCAGACCCCA CCCTTCACCC
  51301 CTTCTCCACC TGAGCTCTTC CTCCTCCACA TCACGGCAGC GACCACAGCT CCAGTGATCA
  51361 CAGCTCCAAG GAGAACCAGG CCAGCAATGA TGCCCACGAT GGGGATGGTG GGCTGGGAAG
  51421 ACAGCTCTGG GAAAAGAGGG GAAGGTGAGG GGCCCTGACC CTGCTAAAGG TCTCCAGAGA
  51481 GGCTCCTGCT TTCCCTAAGA GACATGACAC CCCCATCTCC CTCCTTACCC CATCTCAGGG
  51541 TGAGGGGCTT GGGCAGACCC TCATGCTGCA CATGGCAGGT GTATCTCTGC TCCTCTCCAG
  51601 AAGGCACCAC CACAGCCGCC CACTTCTGGA AGGTTCCATC CCCTGCAGGC CTGGTCTCCA
  51661 CGAGCTCCGT GTCCTGGGTC TGGTCCTCCC CATCCCGCTG CCAGGTCAGT GTGATCTCCG
  51721 CAGGGTAGAA GCCCAGGGCC CAGCACCTCA GGGTGGCCTC ATGGTCAGAG ATGGGGTGGT
  51781 GGGTCATATG TGTCTTGGGG GGGTCTGACG GGAAGAGTCA GAAAATTCAG GCATTTTGCA
  51841 TCTGTCATGG GACACTCCAC CAGCACGCAT GTGGCCATCT TGAGAATGGA CAGGACACCC
  51901 GGGATGGGGA AGAGAGCACA GAACCCAGAC ACCAGCCTGG ACACAGGCAC CTGGGATAAT
  51961 CTTCTATTCC CTGAGAAGGG AACAGCGACT TCTGGTCCTG ACCTGAGTGG AGGCTGAGGG
  52021 ACTCAGAAGT GCTGGACTCA GACCCCCACA CACATTGAGT GTGAAGCAGA GAACAAGGCC
  52081 TGAGAGGAAA AGTCACGGGC CCAAGGCTGC TGCCGGTGTC AAAGGGAACC ACTCATCAGT
  52141 ATTCGAGGGA TCGTCTTCCC GTCACTCCTT CAGAGATTTT ATCCCTTAAT TGTGTCAGAG
  52201 AGCAGGGCGG AACCTCAGAG TCACTCTCTG GTACAGGATC TGGAAACCCA GGAGGATTCC
  52261 TCTCCCTCAG GACCAGAGGG AGGGTGATAT TCTAGTGTTG GTCCCAATTG TCTCCCCTCC
  52321 TTGTGGGAGG CCAGCCCGGG AGATCTACAG GCGATCAGGA AGGCGCCCCG TGGCCCCTGG
  52381 TACCCGTGCG CTGCAGCGTC TCCTTCCCGT TCTCCAGGTA TCTGCGGAGC CACTCCACGC
  52441 ACGTGCCCTC CAGGTAGGCT CTCAACTGCT CCGCCCAACG GGCCGCCTCC CACTTGCGCT
  52501 GGGTGATCTG AGCCGCCATG TCCGCCGCGG TCCAAGAGCG CAGGTCCTCG TTCAGGGCGA
  52561 TGTAATCCTT GCCGTCGTAG GCGTGCTGTT CATACCCGCG GAGGAAGCGC CCGTCCGACC
  52621 CCACGTCGCA GCCATACATT ATCTGGATGG TGTGAGAACC TGGCCCCGAC CCCGCGGTCA
  52681 GCCCAGTCCC CCGAGCCCCG CCCAGCCCCG ACCAACCCGG GGGAATTTTT GGCCTAAACT
  52741 GAAAATGAAA CCGGGTAAAG GCGCCTGGGC CTCTCCCGGG TCAAGGGTCT CGGGGTCCCG
  52801 CGGCTTCGGG GTGGATCTCG GACCCGGAGA CTGTGGGCGA CCTGGCCCGT CCGTGGGGGA
  52861 TGAGGGGTCG TGACCTGCGC CCCCGGCCGG GGTCACTCAC CGGCCTCGCT CTGGTTGTAG
  52921 TAGCCGCGCA GGGTCCCCAG GTCCACTCGG TCAGTCTGTG ACTGGGCCTT CACATTCCGT
  52981 GTCTCCTGGT CCCAATACTC AGGCCTCTCC TGCTCTATCC ACGGCGCCCG CGGCTCCATC
  53041 CTCTGGCTCG CGGCGTCGCT GTCGAACCGC ACGAACTGCG TGTCGTCCAC GTAGCCCACT
  53101 GCGATGAAGC GGGGCTCTCC ACTGCCGGGC CGGGACACGG ATGTGGAGAA ATACCTCATG
  53161 GAGTGGGAGC CTGGGGGCGA GCAGTGGCTG AGACCTGACC GACCCTCCTC CCGGCGCGGC
  53221 TCCCCCGGTC CTGCGCCCCC GCCAGGAGGG CCCCTTGCTT CTCCCCGCAG AGGCGGTTTC
  53281 CCTCCCGACC CCGCACTCAC CCGCCCAGGT CTGGGTCAGG GCCAGGGCCC CCGAGAGTAG
  53341 CAGGAGGAGG GTTCGGGGCG CCATGACGGC CATCCTCGGC GTCTGGGGAG AATCTGAGTC
  53401 CCGGTGGGTG CGTGCGGGCT TTAGAACAGC GACCGCGACG ACACTGATTG GCTTCTCTGG
  53461 AAACCCGACA CCCAATGGGA GTGAGAACTG GGTCCGCGTC GTGAGTATCC AGGATGAAGG
  53521 ACCCTACGTA GGTTGGGAGA GGGAGAAAAG AAACTGCGGA GTTGGGGAAT CCCCAAGGCT
  53581 GTGACTCCCC AATCCATACA CCGCCTTCGG GGCCTGAGAC CCTGAGAGCC ACGCCTGGGG
  53641 CCCTGGGACT TCGCCCTGAC CCCTCTGCTC CTGTGCGAAG CGCTCTGTCT CAATGTCTCC
  53701 CTGAGTCTTG GCCCAAGAGC TGTCTGAGAA ACAAGGGAGA AACCCTCGGT TCACTTTTCA
  53761 TCCCATTATC TCTGTCCCTG AACTGGACTC CCTGACTCCC ACTCCTTACC TGTCCACCTG
  53821 GACTCTTCTA GAAGAAAAAT CACCCCATGG AGTTTGGTGC CAGAGAGTAA GCTTGCTCTG
  53881 GGAATGGAGG TGTAGATTTT TGTTGTTGTT GTTTTTGTTT TTAATCCGGA AAAGTTGTGC
  53941 CTGAGTGCAT GAGACAGAAT AGAGACCAAT TTGCTCTTTG TTTATTAACT ACAGTGGGTA
  54001 GCAGAATCTT GGTAACCCCT GAATGATCAG GAATCTAATA GGTAAAAAAA TGTGACTTTG
  54061 GCCCCTTAAT GTATAAATGT GTCCAAAAGC ATTACAACAG GACTCACAAA GCTACTAAGT
  54121 TGCACTTTCC CAGACAATGT ATCTGTGACT CCTGCTTGTC TTACTTTAAA TTTGCCATCA
  54181 TTCCTTAGCT CTGAGTTTCT GTGTGAGTCC ACGACATCTC CTCAATACAA AGTAGCCACT
  54241 GTGTTCCTAT ATGTTGCAAC CAGAAGCCAT TACAGGCTTT ATTCACCTCA AAGTTGCAAC
  54301 TGTTCAAGGC AGTCACAATG CCCCTCACTA GTGCTCATGC ACTGCCTGTT TTTAGGAAGT
  54361 ATCCACTTCT AAGTGTTGTG TATATTTTAT AGGAACACTT AGTAATTTTT AAACCTGATT
  54421 AACATAAAAA ATAGTTTTTA GGCAGACCCA CATGAGGTAT TAAAAGACAA CTGCCAAGGA
  54481 CACCCTGCTA GGCTCTGTAG ATTGATGTAT TAAAATCCAT AAAACAATGT CTTTAAACCT
  54541 AAGAATTCTG CTGCTTTCAA ATTCTTTCCT CTGCTCCTTT TCCTCACCTC CTGCTTCTCC
  54601 AGCCCTTCCC TCCATCCTTC TCATCCTTCA GGCCCTCCTC TCCCCTTAGT CCCCACCACA
  54661 CTGTCACTCA TGAATTGTGA CACTAGCACT GTCCCATGAC CTGCTACGTA ACTGTTCTTT
  54721 TCACAGTGGC CCTGCTCCTG TGAGTCGGAG TGTGTCATTT CTCCACTTAA AACACTCCAG
  54781 TGTCTCCACC TCGGTCTTGT GAAGCTTCTG GAGTGTCAGG CACTTGAGCA TATGAGGGGA
  54841 TACCTCGTTC ATTGTAGGGA CTAAGTTAAT TTTTGTTGAC TTAATGAATG AAATATGAGT
  54901 GTATTAAATT GCATCACAGA TAATTATAAA CTGTAAAACA CTGAAAAAGT TCAGAAAGAT
  54961 TTTATTTTAT GCAACTAGTG TGTATATCAA TTCATCAATT CATTCGCACA CTACCATGCC
  55021 TGGCAAAACA ACACACATTT ATCTGCTTAT AGTTCCTTGG TCAGAAATCT GGGCAAGATG
  55081 TGGATAGAAT CTCCATTCTG GGCTTCCAAA AGCTGTGTTT TCATTTTGAA TCCTCCTTCG
  55141 GGTTTATACA GAGGTGGCAG AATGCAGTTT CTTGCAGTTG TAAGACTGAG GTCCCTGTTC
  55201 CTGGCTAGCT GTCAATGTAG AGAACTGGGA GGGGCTCAAT TCCCGGTGCT CACCAGCGTT
  55261 CTTTCCTACA CAGCCCCTTA ATTTTTCTTT TTTTTTTTTT TTTTGAGACG GAGTCTGGCT
  55321 CTGTAGCCCA GGCTGGGGTA CAGTGGCCCA ATGATCTCTG CTCACTACAA GTTCTGCCTC
  55381 CTGGGTTCAC ACCATTCTCC TGTCTCAGTC CCAAGTAGCT GGGACTACAG GCGCCCACCA
  55441 CTATGCTCTA ATTTTTTTTT TCTTAGTAGA GACAGGGTTT CACCTTGTTA GCCAGGATTG
  55501 TCTCGACCTC CTGACCTCAT GATCCGCCCG CCTCGGCCTC CCAAAGTGTC GGGATTACAG
  55561 GCGTGAGCCA CCGCGCCCGG CCCATAGCCC CTTCCTTTTC AAAGCCCATG GTGGAGGAAA
  55621 CCCCTCATGC TGAGTCCCTT TCACACTGTA AGTCTCTATG CTCATGAAGA ACCCAGTCCT
  55681 TTCAAGGGCT CACCTGATTA GGACAGTCCA AGCAGGATAA ACCCAGCATA AAGTCAACTA
  55741 ATTGAGACCC TTAATTATAT CTGCTAAATC CCTTCACAGC AGCACCTACA TTAGAGTTGG
  55801 TTGAATAACT GGAGGAAGGT GAATGACCAG GAGCTGCTTG TTGAGGCCAT CATAGAATCA
  55861 GCCTAGCAAG GGTTGGATCT TCCTTTTGTG TTTAATTGGG ACACAGTTGG AAAGTGAAGT
  55921 TCAAGTAACG TGATCATTGT GAATGGTCAT AAAATACATC CTCTTCAGCC ATGGAAATTG
  55981 TCCTTACTTT TTAAAACTAA GTTACATGTT TAATATCTTA TAATTAATTT AGGCCAGGTG
  56041 TGGTGGCTCA CATCTGTCAT CCTAGCACTG TGGAAGGCAG AGGAATGGAG ATTTGTTGAC
  56101 TCCAGAAGTT CAAGATCAGC CTGGGCAAAA CCTCCATCTA TACAAAAAAT TAGAAAATTA
  56161 GCCAGGCATG GTAGTTCATG ATGGTAGTCG CAGCTACTCA GGAGGCTGAG GTCAGAGAGT
  56221 CCCTTGACCC CAGGAGTTCG ACACTGCAGT GCATGGTGAT CATGCCACTG CATTCCAGCC
  56281 TGGGTGACAG AGCAAGACCC TGTCTCAAAA ATAATAATGA TGATGATGAC ACATTTAGAG
  56341 CAAATGCAAT TTGATGTGTA ATAATACATC CTCTCTTGTG AAAATATATT ATTTACTGTT
  56401 GCATAACAAA TTACGTAAAA CTTAGCTCGA AACAACAAAT GTTTCTCATC TCCCACAGTT
  56461 TCCAATCGTC AGGAATCCAG GAGAGGTTTC CCTGAGTGCT TCCTGCTCAG GGCCTCTCAC
  56521 AAGGTTGCAC TCCAGTTGTC AGCCAGGGGC CGCAGCATCT GAGGGCTTCG CTGGGGCTGG
  56581 GAATTCACAA GAAACATGGA TCAGTCACAT GGCTGTTGGA AAAGGCCTGG TTCCTTGTTG
  56641 TCTGTTCCCA GAAGGCCTCA GTTCTCAGTC ATGTGGACCT TCCTGCAGGG CTGCTTATGG
  56701 CACAGCGGCT GGCTTCCCCA AGAGCTCATG ATCCCAGATT CAGAGAGAGA GAAGGTGGAA
  56761 GCCGCAGTTA GTTTTATGTT CTACACCCAG AGTCACAAAC TGTTATGTTG GCACTATTCT
  56821 ATAAGTTAGA AGTTGTATTA GTCCATTCTC ACACTGCTAT AAAGAAATAC CTGAGACTGG
  56881 GTAATTTATA AAGGAAAGAG GTTTAATTGA CCCACAGTTC TGCATGACTG AGGAGGCTGC
  56941 CCCAGGAAAC TTACAGTCAT GGCAGAAGTG GAAGCAAACC CGTCCTTCTT CATGTGGTGA
  57001 CAGGAGAGAG AAATGCACAA TGAAGCAGGG AAAAGCCCCT TATAAAACCA TCAGATCTCA
  57061 TGAGTATTCC CTCAGCATCA GGAGAACAGC ATGGGGGCAC CGTCCCCATG ATCCAATCAA
  57121 CTCCCATGAG GTCCCTCCCC CAATAGGTAG GGATTACAAT TTGCATAACA ACTCAAGATG
  57181 ACATTTGGGT GGGAACACAG AACCAGGCCA TTTCAGAAGT GCCTCATTAA GTCCAAGCCA
  57241 CACTCAAGAG AGGGAATTAA GCTGCACCTC CAGAAAGGAA CAGTATCAAA GGATTTGAAT
  57301 ATATGTTAAA AGCAAAATTA TAACTATTGT TTCAGGTTTC TGAAAATCAA AGTCTTCTTG
  57361 TATCTAATTA TTTTTCATTA ACTCTTTAAG CTTGTCTTTT AATTTAATTT TTTTTTTAAG
  57421 TTCCAGGGTA CATGTGTAGG ATGTGCAGGT TTGTTACATA GGTAAATGTG TGCCATGTTG
  57481 GTTTGCTGCA CCTGTCAACT CATCACCTAG GTATTAACCC TGGCATGCAT TAGCTATTTT
  57541 TGCTAATGAT CCTCTCATCA CCACCCTCCC CCAACAGGCC CCAGTATGTG TTGTTCCTCT
  57601 CCCTGTGTCC ATCTGTTCTC ATTGCTCAGT TCCCAGTTAT ATATGAGAAC ATGTGGTGTT
  57661 TGGTTTTCTG TTCCTGTGTG AGTTTGCTGA GGATAATGGC TTCCACCTTC ATCCATATCC
  57721 CTGCAAAGGA CTTGATCTCC TTCCTTTTTA TGGCTGCATA ATATTCCATG GTGTATATGT
  57781 ACCATATTTT CTTTATCCAG ACCATCATTG ATGGGCATTT GGGTTGATTC CATGTCTTTG
  57841 CTATTGTGAA TAGTGCTGCC ATGAATGTAA ACATGCATAT GTCTTTATAA TAGAATGATT
  57901 TATATTCCTT TGGGTATATA CCCTGCAATG GGATTGCTGG GTCAAATGGT ATTTCTGGTT
  57961 CTAAATCTTT GAGGAATCGC CACACTGTCA CTGTCTTCCA CAATGGATTA ACCATTTACT
  58021 TTTCCACCAA CAGTGTAAAA GCATTCCTAT TTCTCCGCAA CCTTGCCAGC ATCTGTTGTT
  58081 TCTTGACTAT TTAATAATTG CCATTCTGAC TGGCATAAAA TGGTATCTCA TTTGTCGTTT
  58141 TGATTTGCAT TTCTCTAATG ATCAGTGATG GGAGCTTTCT TTTTCATATT TTTGTTGGCC
  58201 ACATGTATGT CTTCTTTAGA AGTGTCTGTT CATGTTCTTT GTCCACTTTT TAATGAAGTT
  58261 TTTTTTCATG TAAATTTGCT TAAGTTCTTT GTGGATTCTG AATACTAGAC TTTTGTCAGA
  58321 TGAACAGATG GCAAAATTTT TCTCCCATTC TGTAGCATGT CTGTTCACTC TGATTGATGA
  58381 TAGTTTCTTT TGCTGTGCAG AAAAAAATTG CCTAATCTGA TTGCTTTTCA TTTACAAAAA
  58441 TGAGAATCTC ATACTTCAGT TAACACATCT AGCTTAGCAT GATGCCAATA ATTTTGGCTT
  58501 GATGCTGTAG CCCGAAATGG TTAGCTCGAA TTGAGAGACA AGTTATTTTC AAAAACTGGC
  58561 TATCCGATGG GCATTCTATA ACACACTTAA CATCTTTTGT TGTGGTATTG AATGAGATGT
  58621 GATAGAAGTG ATTTGGACAC CACTATGATT CTAAATAGCA CTGCTATTAT GTGCTTCTGA
  58681 ATTTTTTTTC TTTTTAGCAG TCCTATTCTG TTTCCATTCA TATTTGCTAT TCCATCACTG
  58741 GGTATTCCTT CCTACTCTTT TCATGTTTCA TTTATTTTGA TCAGTGATGT GCATTTCCAA
  58801 TTCTGTAAAA GTTTAATTCA GTTGTATGTG TGGTAAAATG TAACATAGAA TCTTTGCAAG
  58861 ATGAAATTAC CTTGCACAGC ATATTGAACA TTGTATTATT GAAAATCTAG AGAGATGCCA
  58921 GTGAGCCAAG GATCGAATGA CCTATATGTA GCCAAGGCCG TTTTCATGGC ATCCTGGTCC
  58981 TCTCTGTCAC ATAGCTCCTT ACAATTCTCT GTGGTTTTCT TCCAATGTAA GTAGTTCTTG
  59041 TTAAGAATCT TTGCAATGAA CTTTGAAATT CTTTTCTTTT TCATTGCTAT AAAAAACAGG
  59101 TATATTTTAA TTTGGAAAAC TCTGTTTAAG GAATTATAAT CAAGTAACTA CCAGGGGGCC
  59161 CAGCACTGGT GTAAAACACA AAACTGTAAA ACTACCCTGG GCTGCTACCC TTCGAAATGG
  59221 GGTCTTTTCA AAGTTAGTAA CAATGAAGCT GTCATTCACA CGATGCATGG CTGAACAAAA
  59281 AGACAGAGAC AGCGGGTGCT GCATTTTATT TTTGTAATGT TGCCTTGTCC ACAAGACACT
  59341 ATTTACACTT AAATCAATTA AAATTATATT AATTTTAAAA GTCAGTTTTC CCAGTTTCTC
  59401 ATTTGTACTA GCCACATTTC AAATACTCAG AATTCATGGG TGGCCGGTGA CTGACGTAAT
  59461 GAACAGCACA GATATAGGCA TTCCATCACT ACAGAAGGGA GCTGGACAGC AGTGTGCCAA
  59521 TGTTTTAAGG ACAGAATCCT GAGTAGAATT ATAGGATGAT GACAATTCCG AGTTCCCATT
  59581 CCAATTCATG GTCTCTTCTC TGATGAGTGG TGTAGGGGGA AATCCATCTC TTTTGTGAGT
  59641 AAAGGTTAAT TTTTCCAACT ACAGATAAAT AATTAACATT ACTTTTTTTA AAACCAGTTT
  59701 TACACTGTTA AGTTACAACC AAAAGTGAAG GGCTTGAATT ACATTTACAT TTTAACACAT
  59761 GGTTTTAAAG GGAAGATCCG TGTGAAAAAG AAACAAAACT CAGAAAAAAA ACCCAGAAGT
  59821 TTTCATACCA ATTCCCATAG CAATAACAAT CCCTTTTCTG TCGTTAGAGT TCACCCTCAT
  59881 TCTGATTTCA TAGTAATCCT GTTCATGTCT TTCTTTAAAG CTTTACCCCA TGCATATATT
  59941 TCTAAAACAA CACAGGTTAT CTCAAACTTT ATATAAATTT TGAATTTTTC ATACAAATGG
  60001 AATCATGOTG TGTATATTCT TACAAGACCT CATTTTAGAT CAAATGTATG TTTGCTAAGT
  60061 TTTTATAGGT GTCTGTATTC ATGTTTATTA TTTGTAACAT TTCATTGAAT AAATACATCA
  60121 TATATTAAAT TAACCCTTCT CTCGTTTATG GTCATTTCAT TTGTTGTCAG ATTTTTTAAA
  60181 ATTACAAGCA ATGCTGTTAC AAGTCTTCTA GCACTAGTCT GTTGGCACAA TGGGCACATA
  60241 TCTGTCAAAA TTATACACCT AGGAGTGGAG TTACTGGTAT ATGTGTATGA TATCCCACTC
  60301 AAAGAGATGA TGCCACACTG CTAGATAAAG TGGCTGTGGC AAGCAATGTG TGGTAGATCT
  60361 TGATGGTCTG TATTCTCATC AGTGTTTGGA AAACTCAATA ATTTTAATAT AGAAATTCTA
  60421 GTGCATTTTT CAAAATATCT CATTATGTTT TCCTCATTAA ACTTTATTGA GATATAAATC
  60481 ACATACTACT CACCCATCTA AAGTATGCAA TTCAATGGTT TTTCACATAT TCAGAGTTTT
  60541 AAATCTGCAT ATTTAATTTT AGAACATTTT CATCATCCCA AGATAATCAA CGGCTTTATA
  60601 ATATGTCTCT CTATACAATG AAGTAAATCC ATAAATTTTG AGTTTTCACT TCAGGAGTGT
  60661 TTTTTCTATC ATTGGCAACT TTCCTAGCTT GACAGGCACC TTTCCAAGTA CCTTTAACAA
  60721 TTTTATTTTT AATAGAGATT TCATTCAGTA ATCAGATTTG GAGAGCACTG ACAAATTTTG
  60781 CCACATTGAG ACTTATAATT CATGAACATG TAATGTTTAC TTATTAAATA CTCTTAAATA
  60841 AAGTATTGCT GGTTTTACTT GTGCATCTTT TAGTAGATTT ATTCCAAAGA TCTTGAAATA
  60901 TGGTGCTAGT GTTCACAGTA TCTCTTTCTT TGTTGAATTT CAGTGTTGGT TGTGGTATGT
  60961 ATAAATATAA ATAATTTTAT ATAGTTTTAA TTCAATGACT TTGCTTAAAG CTTTTATTAA
  61021 TTCATGTAGT TGATATATTC TGTTGCCTGA TTGCACTAGC TAGGAAATCC AGTGTGATTT
  61081 TGAGTGCAAT GGAGAGGGCA GGTCTGCTGG TATCTTCTCT GTGCACTCCT GATCCACACT
  61141 CCATGTACTC TCCCTGCTCC GTGTCCTGGA CACTGTGCTG CATGGCCTGC TGGACCACAA
  61201 AAGGCAGCCT TGCTTTCTGG CTTTTATTTG GGATCGGCTA AATGGGAGCA TCATCACGGA
  61261 GAGAGTGATG AGGAGCACTG GTTGGAGGGA TTTCTCCCCA TAGCTTTCAG TATTGACATA
  61321 GGGAGAAATG AGATGTGATT GGCCTGGATT CATCAATATG GGATCTTCAC CCAGTATCTT
  61381 AGATTAAATG TGTTATTTTG AACACCTAAG AGTGATGTTA ATAGTCAGCC AGGTTAGCTA
  61441 ATTAAAACCC GAATTCAACT GTGGCCTAGC ACAGTGTTTC TCAAATTTGA CTGAGCTTTC
  61501 CATCACGTGG GAGATTTTAA ATAGCCCCAG GTCTAGATCA ACATGTAAAA CCAATGAAAT
  61561 CTAATTCATC AAAAGTAGGA TCCTGGAAAC AGTATTGTTC CTAAAACTCT CCAGGTTTGA
  61621 CTCCAATCAA CAGCCAAGTT AAAAACTAGT AGCTCAAAGC AGTGGTTCTC AATATTTGGT
  61681 ATGTATCAAA ATCATCTGGG AATTGTAGCA AAACTACACA GGCCCAGATA TTATTCTAAT
  61741 TTAACAGGCT AGAGCCTTGA GTTTATTAAA ACAAATCATC TCTCCAGATT TTGCTATATG
  61801 CAACAGTTTG TGAAAACCAC TAGCCTATAT CAACAGAATC CCTATCACAG GCATACTGCA
  61861 GAGGAAAGTG TCTGAACACA GCTCAGAGAG ACGGAAAAGT GGGAATGTAT CCATTATGTG
  61921 CAACCTACTT AACATCTCCT AACCACATCT CCCGGGAGGG CTGAGAGGAT GCCCCCCTTA
  61981 CCAAGGCATT AAGGTGCCAC ATGACACATA CAGGTATAAA AACTGCTAGG TCTGCTGAGC
  62041 AGTCACCAAT CTTGAGTCCT GGATATTCAG AAATTCTTAT CCAGTAGCTA CACCAAAGCC
  62101 AGCTGATATG TCAGAGCCCC TCAAATAAGC AGAAGGCTGG TGCCCTTGAG AAAGGACCCT
  62161 ATAATTCACT CTGAGATAAA TTCTTTTTAC AGGACTTTCC CAGAGGAACA TTTAGTCATT
  62221 TATCGCTGTC CACTGGGGAA TGGAAAACCC CAAATAATCT GGGAGCTCAA CTTTAATGTC
  62281 TTTAATCCTG TCAAAGATTA CCCTGGGGTT AATACTTTGT TGTAACCTGC ATAAAAATCT
  62341 TTCCCTACAC TAAAGTAATG AAGATATTCT ACTATATTAC TGTAAAGAGA TGAGTACAAT
  62401 CACATTTTTT TACAAGAGAT GAGTACAATT GCATTTTTCC AGATAGATAA CCAATTGTTG
  62461 TTAAATTATT TTCTAAATCG TTTTCCTTTA CCCACTGATC TGTAATGTTG CCTCTGCTCT
  62521 ACATACGTTC AGGGCTTTCT ATTCTGTTCC CAAGTTCTAG TTTACTACCC TGTCTCAACA
  62581 TCTCGACTCT CTTAATTAAA ATAACTCTAA CATAAGTCTT AATATCTGCT AAGGAAATTC
  62641 TCTCTGCTTA TTATTCTTAT TTAGAGTGTC ATAGCTACTT TTTCTCTTTC ATATTTCAAT
  62701 ATAAGGCTTA AAATTGGCTT ACTATGTGTT GCAAAAATGC TTGATAAACA TTTGATTGAG
  62761 ATGTGTTTGA GTTCTTAGAG TACTGTAGGA AATTAATGTC ATTATTATAT TGAACCATCC
  62821 AAGCCAGGAT CCTGGTAAAT TGATTCAGTT AATTCACTGT CTTCTTTAAT TACTGTCTTC
  62881 TATTATTACT CAGTAGGGTT TTTTAAATAT CCCAATTGAT ATCTTGTACA GAATTTGTTA
  62941 GGTTTAGTTT TAGAAGTTTA TATGTACGCA CTTTTGCTGA ATCAATTTTT TAAAATTTTC
  63001 ATTTTATATC TGTGCAAAAC AGCTCTATAA TTTCTTCTAC CTACCAAGCA AAAATATCTT
  63061 ATTAACTTCC GGATCACGAG GTCAGGAGAT CAAGACCATC CTGGCTAACA TGGTGAAACC
  63121 CCATCTCTAC TAAAAATACA AAAAATTAGC CGGGCATGGT GGCGGGCGCC TGTAATCCCA
  63181 GCTACTCGGG AGGCTGAGGC AGGAGAATGG CCTAAGTAAA TCTGGGAGGC GGAGCTTGCA
  63241 GTGAGCCGAG ATCTCGCCAC TGCACTCCAG CCTGGGCGAC AGAGCGAGAC TCCNTCTCAA
  63301 AAAAAAAAAA AAAAAAAAAA AATATATATA TATATATATA TAAATATATT TATATATATA
  63361 TGTATCTTAT TAACTTCAAG AGTGTTCTAA TCATTTGTAT TTTCTGCATT CAGCATATTA
  63421 CCATTTGCAA ATTATAAGAG TTTTGTGCCT TCTTTTCAAA CCTTTTTAAG TATATTTCTT
  63481 TTCTTCTTTA TGACACTGGG AAGATCCTCA ATTACAATTT TTAATAAGAG CAGTGAAAAC
  63541 TGCTGGACCA CTCAAACAAA AATTGAGCTG CCACTCCTGA GAACTGACTG TGTTGCTCTT
  63601 ATGGATCAGC CCTTTGGGAC CCATCCTAAT CCCAGGGATT TACAGTGCCA CATATGCACC
  63661 CGCAGCCTTT AGATGTAGAC TTTATCCACC TAGCCCTCAA AGGTCTCAGA CACTGCCCAG
  63721 AGCAGCCAAC ATCACCCAGG ACAAAAGCAA CCCTGCAGCT CAACTTGCTC TTCTAGATTT
  63781 CTGCCTTCTC CTGGTTCCTG GCCTGGCAAA TCTTTACTGC TTGGTTAGCC CCTTCATACT
  63841 TTCCATCAGA TTTTTCAAAT ATGTACCCTT TTCCCACTTG TCCAGTAGTA CTCAATGGGA
  63901 GGGATGATCC AAATTACATA ATTAATAATT GOTGAAAACT AAAGTACAAT GTACAATTTT
  63961 TATGTCAATG TCACATGCCA AGGGAGAAAA TGCATAGGTA TCAGTAGTGA CTACTTAAGC
  64021 ATGTGTAACT TAGAGGTAAT AAAAACAAAA ATGTTAGGTA ATATCTATCT ATTCCAGATT
  64081 AAAATCTACA TCAATAAATT CAAACTTCTA GGAAAAATAT CTGCATATGT AATATATGAC
  64141 TGTGACTCCG ATAGACGAAC GTGGGAATAT AGTATGAAGA TACTTCTCAT CGTCTTCACT
  64201 CTGTGAGGCA GTCAGTGTGA AATGAGAACC AGGGATTCAA AATGTGTTGC AAGACTATGA
  64261 AAAGCACTAG AGTAAGTTCT ACACAAATAA AGCAATCTCC TCATAAATTA CACTGGGCTT
  64321 ATCTGTGAGA TGCATGTCCT TATCAAAATG GAGTTAATTC TAGGTAGGTG ATCTTGTTTA
  64381 AATAAAATGG ACTCTGACTC ACTAGCTACT GGTCCACCAT CGGTTTTTCC CTTTACTAAA
  64441 AGGGAGGCCC CACACTGAGC CAGCATTTCC CAAGAGGAGG TGTCTTGCTG ACATCAACTT
  64501 CCAACAGGGT AAATTTTCAT GGGAACCCAA AGAGAAGAGG AAGACCTCAA GAAAAAGGGC
  64561 CTAAAGAAAT GACAAGAATG ATAACATTTT ACACTTTTAT TGTGCTATGT GTCAAATAGT
  64621 GGTCTTCGCA CTTTAAAAAT GCCAATTCAT TTTATTCTGA CTACACATCC ATGGCTTGTG
  64681 CCTGTTATTT TTCCCTTTCA TAGTTAATGC CATAGGAATG AAGATCACAC AAAAGTAGGG
  64741 ATGGAGCCGG GTTTGCACCC AGACAATTCT GCCCCAGGGC CATGCTCACA CCACTGCACT
  64801 TTTCCAGAAT GTGAGGGTGG GCGGAGAGTC CAGCTCAGGG AGAGAGATTG GAGTGAGAGA
  64861 AGAAGGAGAG GGGGCCGACT GGATCACTGT GATGGTCTGG ACCCACGATT CCAGGAGAGA
  64921 AAGACAAGGA TTATGTCACT CAGGGAAAAT ATCCAAAGTC TCTGGTTTAA ACTTAAGCAT
  64981 CTCCAGCTCC AGGACAGGTG GCTGGTGGGA AGACAAACTA AGCCAAAGCC ACAGCTCTGA
  65041 AGATTTCCCT CTCCTGAGAA TTCTGCAGGG GTTTCCCTGA CCTCATGGCC ACCTCTCACC
  65101 CTTGGCTCTT GTTTTTCCCC CAGGACTGAT AAGGGCGTCC AGATGGGACG CCTTTATCAG
  65161 TTGGTCTCGG GTTCTAGAGA AGCCATCAGA GTCAGTGGAG GATGAGCTTC ATAAAGTGGG
  65221 AGATCTCCAG GATCCTTCCT GGACTCCAAG ATACCCAGAG AAGCCGGATC CTGTGTCCCG
  65281 GAACCCCCTT CCTGTTCCTT TGTGGGCCCT GACCTTAGGG AGAACTGGGC TGGTGAGAAG
  65341 ATCAGGATGA ACTGGGCTGG GGAGGCAAGA GGGAAAGGGC GGCTTGGAGG GCTTAGTAGC
  65401 TCCTCTCGCG GCGCCTCCGT CCCGGGCTGG GATCTGCAGA CTCCTCAGGT CACTTCTCCC
  65461 AGAGCCGCCG CCCCAAGCCA CCCTCCCCTG GTTCCCGTCC CTCTGTCCCC TCCCCAGCTC
  65521 CCCCCACACA GTAAGAAGCT CCCAAGTGAG CGGCTCCGGT GCCGGGCGGG ACGTGGGAGG
  65581 GAGGCTGTGT CCCCGCGGGG AGGCAGGGCG ACGGCGTCCA GGAGAGAGGG GAGCGGGGCG
  65641 GTGTCCCAGG CACCCTGCGC CTCTCCCGCC CGCAATGTGC GGGTTCCCGG AGCCCAGGCC
  65701 CGCACCCCGG GTACTTGAAG GCCGCCGGGC TCCGCCTTCA CCACGTGGGC CCTGAGTTCC
  65761 TCCGCGGCGC CTTCGTGGGC GACACGCGAT TCCTGAGCTT CCACAGCCGC GGCGAGAATC
  65821 AGAGCGTGAA GCGCGGGTGG AGAAGATGGC ACAGGGCTGT GGGCGCCCTG AGGAAAGTTC
  65881 TCGAAGACCG CAGGGAATGT GTTACTGAGG GGCCTGAGGT TCCTGGGCCC GCACCACCAC
  65941 CAGGGTCAGC CGGTGAGTGA CCTCAGACTC CGGGCACAGG TGTGCCCCGC CCCGACCCCA
  66001 GAGTGACCAG CCCGGTTCTC CCAGGGATGG GGGGGCGGGG CCTCAGCTGA TCCCTCTCAG
  66061 GTGGGGAAGC GCTGGGGCCA TTTTGCTCCA GGTTTCTCCT TCAATCTGGA TCATGACTGA
  66121 CTTGTTCTGT GACCAATGAC AGAGGGAGGA AGGAGCTGGG GTGGGCGGGG CGACCTCCCT
  66181 CCCCTCCCTG GCCCCGCCTC CGGCTCACCC TGGGGCAGGT CCAGGGCACC GCACCTGCAG
  66241 ATGCTGCTGT CTGGCTGGGT GGTGGCTCCG GACAGGAGCT TCCTCGAAGG ACACTTTAAG
  66301 TCCGCCTACG ACAAGGAGAT TTCATGTCCC TGAACGAGGA CCTGCAGACC TGGACAGCGG
  66361 CTCAAAGAGC GGCTTGGAGG ATCCATCACA CCTGGGAGAG GTTCTGGACC GCGGAGGCCA
  66421 CAAGGGAAGC CCCGATGGGT CACTGCACTA GGTGGCTGCT CAGACACCTG GAGGATGGCA
  66481 AGGAGATGAT GCTGCCGGCA GGTACTGGGG TCCTCCAGGC CCCACGTCAG GACCTCTTGG
  66541 AGCTGGAATT ATGTTCCTTG GAGGCATCCA TATTTGAGGA AAGAGGAGAC CGAACAGTCT
  66601 CCGTCTCCTC CTCCTCCTCC TTCTCCTCCT CCGTCGCCGC CTCCTCCTCC TCCTCCCTAC
  66661 TGCGGGGTTG GGGATTCTGG CCCAGGCTCC TGAGGCTCTC CCATGGGTGG GTGGGGAGGG
  66721 AGGACAGAGA AGAAGGAGTG CAGGGAGGTA AATAGGAAAG GACAGTAGTT ACCCTTGTCA
  66781 ACAGTGATGT ACTTGGGTCT AGCTTCACCG GGAATCTTTT TCTTTCCTGG CCCTCTGTGC
  66841 CCAACAATTA CATATAATCT TTAACCCAAC CAGAAGATAA AACACCAGGA AACGGAATCG
  66901 GCAGAGACAC AGTAGATGAA AGTACCACGA AGGACTGCTA GGTTCTAATA GAAAGTAGCA
  66961 GATGTGAGTA TCAAAAGTTA TAAACCTTTT TGCAAATCAG AAAAGAAACA GACTTTTTTT
  67021 TTTTTTTTTT TTTTTTTTTT TTTTTTTTGA GACGGAGTCT CTCTCTGTCA CCCAGGCTGG
  67081 AGTGCAGTGG CGCGATCTCA GCTCACTGCA AGCTCCGCCT CCCAGGTTCA CGCCATTCTC
  67141 CTGCCTCAGC CTCCCGAGTA GCTGGGACTA CAGGCGCCCG CCACTAAGCC CGGCTAATTT
  67201 TTTGTATTTT TAGTAGAGAC GGGGTTTCAC TGTGTTAGCC AGGATGGTCT CGATCTCCTG
  67261 ACCTCATGAT CCGCCCGCCT CGGCCTCCCA GAGTGCTGGG ATTACAGGCG TGAGCCACCG
  67321 CGCCTGGCCG AAAAAGACAT TTAAAGACTG AAAAGTGGTA TCACCAATGT TAGAACCAAA
  67381 AAACTGGACC TGAGGGTGCA TCACCCGAGG GGGTTAAGTT AAACTCTGTC CCTGTTCCTT
  67441 CTAGAGCCCC GTGGAGATTC CTGCTGTGCA GCAAAGCCTG GGAGGGGCCC CTGGCTGTGG
  67501 AAGGCAGGCT GAGACGTCAT AGGTGAGATA GGAGGAAGGG GACCTGCTGG ACTCCCGGGG
  67561 ACCTGCTGGA CTCCCGGAGG CCTGGTACCC AGGGAAGGCC AGTTCCTAGG GCTGCCTCTG
  67621 GACTTCAGAA CCTTGATGTT AAGTCATATT TTCCCGCTCC TCCATGAGCT TCCAGGAGCA
  67681 GGGCCACATG GGGCTCAGAA AGTCCCACAG TGCCTAGGAA GTGGGTCTGT CTCTTGTGGG
  67741 ACAAAAATTC TCAGGTCTTT CTCTTTCAGA TTCTTAAATT TCTTAAAAGA GATTGAACTG
  67801 GAATTACCCA GGCCCCAAAC TGAATCAAGG AATTGGGACT GTGTAATGTG CGTGTGTCTG
  67861 TCTGTGCACA TGTGGATGTA TAAACAGGTA TGGGCATCTA TGTGTATGTG CGTGTTTATA
  67921 AGTGCGTATA TGTATGTATC TGTACATATT TATGTATATA AGCAGGTATG GGTATGTATG
  67981 TGTGTATGTG TGTGTGTTAC TCTGTGACTT CCTCATACTT GGCACCACCC TGGCCTTTTC
  68041 CTGGTTGCCC CCCACACCAT AGGTTGTAAG GGACAGAACA GCGCTCCACC TGCAGACATA
  68101 GAACGGGGGT CAGGATAAGT GTTGGGGAGA GCTGGTTTCA TCACACCTGG AATAAGGAAA
  68161 CAGGACCTAA ATGGATGGGT CCCACTCTTT TACAGAATTG TACCTTAAAA AAAGTGTCAG
  68221 TCACGTTCCT AAGGGATTTA CATGAATTAA ATCATTTAAA CCTCACAAGA GCCCTATGAC
  68281 CTAGAGACAG CTATCAGCCC CATTTTACAG ACAAGGAAAC TGAGGTCACT AGGAAGTTTA
  68341 GTAACTGTCA GTGGGCTGAC TCATGACTCT AAGGAGACAG TCTGACTGCT GAGCCCAAGT
  68401 AACTGCTCTG TCATACTCAC CCTCACACCC ACCTACTACT GGCTCCCTGT GGGTCACCTC
  68461 CTCCTGTGGG CTCTGACCAG CTTCCCTTTC TTCTCCAGGC ACAGAGACTG GACTGAAGTC
  68521 TATTGCATTT GCCACAGTCC CGACTGGGGA GTCATGGGGT TGAAGTGACC CATAGGCTGA
  68581 TGGAGTGTGC TTGCTGGATG CGGGGGAAAG GGAGATTTAT ATCTGCCCTG GAGGGAGTCA
  68641 ATGGTGTTTG CATCATGGCA TATGAGGTGC ACTATCTAGA ATGCTGAAGG GTAAAGACCA
  68701 GCAGGGGCCA TGCAGAGTCT ATTCATCACG GTTTTATTTC ACAGCCTGCA TGGGCCTGAG
  68761 AGTGGTGGGG TGTTCACTCA TCTCCATCCA ATCCCTACTG GTTCCTGACC TCTCTTCTTC
  68821 CAACTGGTAA TACCTGCACT CAAGGTTGAA CCATGAGGAG GAGTCTACCC ACACCCCACA
  68881 CTTCCTCCTC TCTGCCTGCC ACTCCCTAGT GGGGCCTCCT GGATGGTGTG AGAGGAGAGT
  68941 GACGGAGATA GTAAGAGAAC TTGACATTCA CACTGGTAAA GAGAAAGTGG GATGTCAACC
  69001 TATGAGAAAG ATAAGGGCTG CCACAGGGAA GGGGATGAGA TAAGTTCTAA CTGGCCCCAA
  69061 GGGTCGCTTA ACTCAGTGAA AGCTGTGGGA GGTGTTTTCA GCTATCCATT GCTATGTAAG
  69121 AAATTGCCAC AAACTTAGCA GOTTACAACA ACACACATGT ATTATCCCAC ATTTCTATGG
  69181 GTCAGGAATC CAGACACAGC TATGCAGCTT TCTCACATGA TTTTCATCAG AGTGCCATGG
  69241 CTGGGTCTTA TCTGGAGGCT GAACTGGGGA AGGATCCACT TCCAAGCTCA AGCAATTGTT
  69301 GGCAGGATTC ATTCCTCCAT GCCTGTTAGA CTGACAGCCT CAGTTCCTTA CTGGCTGGTG
  69361 GCCAGAGGCT GACTTCAGTT CCTTGCCACA TGGGCCTTTC TGCTTCATCA AAACTTGCAA
  69421 ACAAAGAAGG TAGAAGACAG AGTCATCTAG TAATACAGAA GTGCGGCTGT AATATGACCT
  69481 CATTACGGAA GTGACATCAT TTTTGTCACT TTCTATTGGT TAGAAGCAAG TTTAGGCCCC
  69541 ATCGGTTCTC AGCTGGATGA GATGCCAAGA GACAGGATCA CCGGGCATCA TCTTAGGGTC
  69601 TCTATACACA GGCTGGACCA TGACTGAACA AGATCATTAC AAACCCGGGT CCTGGACTGG
  69661 GCCAGATGAT CTGCAGAGCT GAGCTGTGTT TATGAAATCG AATCCCTGGA AACAAAAATC
  69721 AAGAGACGCC CACTGGAAAG GCTGGTCTTG CATTCTGTGT GAAAGCTAAG ATGGCGTTCT
  69781 CTCCAAATGT TTTTCTCTAT TACTTTTCAA ACCTCCTGCA TCTGTAAAGA AGCAACACCA
  69841 CTAAGCCTAT CTGGTTTTTT TTTCATTCGA GTGTCATACT GTTAGGCAAA TCTTCTGCTT
  69901 TCACTATTTC TTCAAGCTTG TTTTCATGCA GAATCCCACG AACTTCTGGT AATTACACTT
  69961 TATTGATTTC TTTTCCTTCA CTCAGTTTAT TTTTTAGTGA TTTTATTTCT CTTGTATGGA
  70021 TAAATTAGTC CATTTAATTA GGGAATTTGT GTCTTCTAAA GAAATATTTC TACTATTTTT
  70081 TGGACCATAT GGGCTAAGAT CCTCTTGCCA AGCCTCAGCT CACTTCCCAC ATGGACACGA
  70141 CAAAATTACT GGGGGTCCTG GAAAGTCGGG GATTGGCATA GTTCTTAAGC AGGATCAGAC
  70201 ATGTAAGTCA CAAAGATATG AGGCAGATAC CTTTGATTTC CTTATGACCC TGATGACAGC
  70261 ATACCTGGCA CTCAATACCT GTCTGTAGAG GTGAACTCAA CTAAACTCCC TGATCACTCA
  70321 ATACCATGTG AGCCCAAGGA CCTCTTATGT CATGGGCCAA ATTCCCTTGT TTTATGCCAT
  70381 TTTTCCAGAT GGGGTGCTGT TCACATGGGG ACGTTTCTCC TTTTCCACCT CATTTTCCAT
  70441 GTGTGAAACA CTGGGTCTTG ATTACTCAGA TCTCTCAGGG TCCTTTCTAC CTTTTCCTCC
  70501 ATCCTAACAT TCTCCCTACA GTTCCCGCCA GGGTTTCCCC GAACCTCTTC TTCTCCATGT
  70561 ATTCAGTCTA GCATCTCTCA GTTCATTCTT CACACTTCAC CAAAGTGATA TTCTGGAATG
  70621 CAGATCTGAT CAAGGTTCTT CCTGACTTCA GTGCTGCCAG TGCTGCACAT TGTCCTCGGA
  70681 TAAAAGCCAA AGGTTCTCCC CAACCCCACA AGCCTTAGTC ATTGAATCTG CTCACTGCTC
  70741 CAAGCTTGCT TTTTGCCCCT GTGCTCCTCT TTTTCTAATC TCTACCCTGA ACTGCTGACA
  70801 GTTCCCCTAG ACTCCCTGTG TGTTCTTTCT TCTGAGCCTT TGTTTGTGAT ACTGATTCCC
  70861 TAAAAGATTT CTCCTGCCAT TCACCTGGAT TAACTGTGTT CCTCATCCAG TTCTCTGTTT
  70921 AGACATTGCA TCCTCTTGGA AGCAGTTTTC CCGCACTAAT TATAGTAGCC TGCATTTAGC
  70981 TGTTTTTTTA GTCCCCTTGT GGAATCTTTG ATGTTTCACT CATTGAGGGC TAGGAGACCC
  71041 TGACACATAG TAACTGCCTA ATAAATGTTT GCTAAATAAA TGTGGGGTTC ACCATGAGAT
  71101 ACCACGTTAT ACATGCAAGA ATGGCATTAA TTTGGCTGGG CGCGGTGGCT CAAGTCTGTA
  71161 ATCCCAGCAC TTTGGGAGGC CAAAGCAGGC AGATCACGAG ATCAGGAGAT CGAAACCATC
  71221 CTGGCTAACA CGGTGAAACC CTGTCTCTAC CAGAAATACA AAAAATTATC TGGGCGTGGT
  71281 GGCATGCACC TGTAATCCCA GCTACTCGGG ACGCTGAGGC AGGAGAATTG CTGGAACCTG
  71341 GGAGGTGGAG GTTGCAGTGA ACCAAGATCA TACCACTGCA CTCCAGCCTG GGAGACAGAG
  71401 TGAGATTCTG TCTCAAAAAA AAAAATGGCA TTAATTAAAA AGTCAAAAAA CAATAGATGT
  71461 TGGCATGGAT GGATTACACT GCTGGTTGTG AGAATGTAAA TTAGTACAAC TACTATGGAA
  71521 AACAGTATGG AGATTCCTTA AAGAACTAAA ACTAGATCTA TCATTTGATC CAGCAATCCC
  71581 ACTACTGGGT ATCTACCCAA AGGAAAATAA GTCATTATAT GAAAAAGACA CATGCACACG
  71641 CATGTTTATA GCAACACAAT TCATAATTGC AGGGATGTGG AACCAGCCTA AGTGCTCATT
  71701 GACCAACCAG TGGCTAAAGA AAGTGTGGTG TATGTACACC ATGGATTACT ACTCAGCCAT
  71761 AAAAAGGAAT GAAATAATGC CTTATGTAGC AACTTAGATG GAGCTGGAGG CCATTATCCT
  71821 AAGTGGAGTA ACTCAGGAAT GGAAAAACCA AATACTGAAT GTTCTCACTT ATAAATGAGA
  71881 GCTAAGCTAT GAGGACACAA AAACATGCAA AATGACATAA TGGACTCTAG GGGCTTCTTG
  71941 GTGGAGGTTG GGAATAGGGT GAGGGATAAA AGTCAACACT GGGTAGAGTA CACTGCTCGG
  72001 GTGATGGTGC ACCAAAATCT CAGAATTCAC TACTAAAGAA CTCACACACA TAACCAAAAA
  72061 CTACCTGTCC CCCAAAAACT ATTGAAATAA AAAAATTTTT TTAAATAAAT GTGGGGTTGA
  72121 TTTGTAATTT AAAATTTATT TTGAATAGCT AATACAAGAA CAATATTTAA AAGATTATAA
  72181 TAATAAAAAT GTGTATAGTG TGCAACGCAA ATTTCCTTCT CCACCCCATC CACAATTCTT
  72241 CCAAATTCCT TGCCTGGATG CAACCACTGT TACCAGTTTC AGCAAGAAAT TAAGACAGAA
  72301 ATTAAAGTTC TGCTCCTCAT AATAATCACG ATCAATTATC CCTGCTTATA CAGTAACCCC
  72361 TCCTTTTCCC AACAGCCTCC TAGCATGAGG AACCCAAATT GAACAGGTAA CAAACATACC
  72421 CCTAAACGTA ATGTAACTTG TACTCTGTTC CATGGTAGGA ACTTCCTTCT CTGGGATCCC
  72481 AAACTCCTAG ACCAGCTGAG TACGTTGAGG GGATGGGAAG CACCAACACC CCACGCAGGT
  72541 TACCGGAAGT GCTGGTGAGT ATAGTCATTC CTTCTTCCAG CACTGGTGGT TAGAGACTTA
  72601 CTCTACCTGT TGGAGACTTG GCACCATATA ACAGCTGCTG GCGTAACCTT AGTGTATGCT
  72661 TCATCCTGAA GGACATTACC CATCCTGATA GGATGTGATC TTCAAGATAA AACCCAGCTG
  72721 CACCCTCAAG AGACCATTCC ACTGCTCTAT CAGCTCGCAG AGTGTGTGAT GCAGTATGGG
  72781 GTAGAACCAG GAGATCTCAT GGTCATGTGC CCACTACCAG ACATCCTTCG CTATAAAGTA
  72841 GGTCCCTTAG TCTGGGGCAA TGATATGTGG GTCCCCTATT AGAAGATCAT ACATATGTGA
  72901 GCCCTTAGAT AATGTTTCTG AGTGTGGCAC TGTGGGAAGA AAGAACAAAC TCATACCGCT
  72961 AATACAAGGC AACCTAAATC TGACAGAACC CCTGCCTTTT TCATTGTGAA AAAGTAAGAA
  73021 TAGAATCAAT TTGCCATCAA ATTGCTCATT GGCTTCCATA AGCGAAGGTA ATCTGTTGGA
  73081 GTTTATTTAT CGCTGGAAAG TAGATTGTGA CAATAACCAC GTCAGCCATG CTGAAGGCAG
  73141 CCCAAGCCTT GGGACCCACC CACAGTGCAT CTCTTCCACC ATAGATAACT GCTTTCAAGA
  73201 GCCTGTTTCA TGAGCACTGG GATGGTTAAG GACAGAGGCT GGCTGATGTG TACCGAATGA
  73261 TTCATCCTGG ACACCTGGTT GTTTAGACTA CTTGTATAGA GGGTAATTTT TGCTGAGTAG
  73321 TAACATCTGA TACAAAAGAT CCAAGCACTT TGTGTCTACT GTATGCCTCT TTCCAGATAT
  73381 TCCTGTTCCT GATCTTCCAA TCTTGCTCCT TCCAGGCCCC TGACCTGCTA TTTAAGCCAC
  73441 TTGCTGTTGC CTATAAGTCT GTGCATATTC TCACCTCAGG CCACTTCTGT CTACACAAAG
  73501 TGGGTGAATC AGAGCCCCAC CTGCCTATTG GCAGGACCCT CTTCAATGCT GTCTCTACTC
  73561 ATTCCTGAGT TGCCCACACT GCAGACACAC TCTACTGCTG CCTCACACCA ACACACTAAG
  73621 CTGGCATGTT TATGAAGAAG CTCAGAATTT TCCTCTTAAG TCAGCTGGTC ATAGAAACTA
  73681 CCCATAAGGT CATTGGTGAG AGTCGAGGTC AAGGCAAAGG TCCAGCAGTG CGAGGTGACA
  73741 TGGGGTCTGA GCAAGGTCTT TACGTAATTA ACACCAGTAC TCAGAACATA CGTACTTTAA
  73801 CCCAGGGGTC AGCCAATGGT TTCAATAAAG GCCCAGATGG TTTCCCTTTG CTGTGTGTGT
  73861 GTTCATTCCC CTCTCCGAAG CTATGTGTAA CCCCATAGAG ATCACAGGGG TTCCCATCAA
  73921 CCACAGCCAG TTACATCCAC AAAAACCTTG ATCCTTGTTA GCAGATGGGA GAGGGTGGCT
  73981 TGGAGGATTG AAATCCCTAC TTCATGGGAC TCTGGATACT TACACAGTTG CTTCCTTCTG
  74041 TGTGTCTACA TGAATCTCAG CACTGTGTTT GGGACACCAT CTACACTCTT ATTCTTCATG
  74101 CAATTCTGTT TGCTCCTCAG ATGGCCGCTT TTCCTTTGAA GCTCAACAGT AGCCATCACG
  74161 TACTTGGGCT GAGGGTTACT GATTAACTCT GTGGGATTCT GGGATCAGTT CCTTCCCTTT
  74221 CTTAGCCATC CCTGTGCTTT ACAAAACTTA GCTTCACAAT TATACTCATC TCTCCCTGTG
  74281 TGCACCTCTA AGAAATTACT GATAAAACAC GAGTTTTTTT GAAATTACAA AAATATATGC
  74341 AAGAAAATGT TTATGGGGCC AATTGATCAG CACGGAAAAG CATAAACTTT CCTGTTTTCT
  74401 GCCTCTCATT AGAATTTAAT CCCCCAAAAT TGAACTCATT CCAAACTTCT GGCTTAACTA
  74461 GGAGTTGCTG TAGCCAGCCT ATTCTAGACA GCATCCTGTA AGCCCCTGGG ATTGGCAGAC
  74521 ACAAGACACT TTTTCCATTC ATATATGTGG TTCTGTTCTG GCCAGATCAG TGGACTTCCA
  74581 GTGCCCTTCT TGAGTCATAC TGAGGTAAAT TACTCAGACC ACGAACTCAC ATTTTTAAAT
  74641 AAATAAAATA GCAGCATAGT TAAGAATTTA TTTATCCCAT TACTGGGTAT ATATGCAAAG
  74701 GATTATAAAT CATTCTACTA TAAAGACACA TGCACACATA TGTTCACTGC AGCACTATTT
  74761 ACAATAGCAA AGACTTGGAA CCAATCCAAA TGCCCATCAA TGATAGACTG GACAAAGAAA
  74821 ATGTGGCACA TACACACCAT GGAATGTTAT GCAGCCATAA AAAAGAATGA GTTAATGTCC
  74881 TTTGCAGAGA CATGGATGAA GCTGGAAACA ATCGTTCTCA GCAAACTAAC ACAGAAACAG
  74941 AAAACCAAAC ACCGCATGTT CTCACTTATA AGCAGGAGTT GAACAATGAG AACATGTGGA
  75001 CACAGGGAGG GAAACATCAC ATACCGGGGT CTGTCAGGGG ATGGGGGCAA GGGGTGGGAT
  75061 AGCATTAGGA GAAATACCCA ATGCATGCGG GGCTTAAAAC CTAGATGACG GGTTGATGGA
  75121 TGCAGCAAAC CACCATGGCA CATGTGTACC TATGTAATGA ACCTGTACAT TCTGCACATA
  75181 TATCCCAGAA CTTAAAAGTA TAATTTAAAA AAAAGAATTT ATTTCATTTG TATANNNNAT
  75241 GAAATTTTTT GGACATTTTT TAAACCTGCA CTTTTCATAG TTTTTGATAC ATCCTTGCTC
  75301 CAATAAATAA TTTGCTAAAA TTACTAAATC TCATAGATAT ACCAATGGTT TGCATCAGCT
  75361 AATGGCATAT TTGCTTGTCA AACTGAGGTC AGCCAGTCCC CCTTCATTCA CTGTTAGTAA
  75421 CTCATGTGTT GCATTGTTAA AAACCCAGTA TCACCTCATC TCCCCTCTAG CATCTTCTCT
  75481 GCAGAGGAGA AGCCAGGCAC TATATCTCCC AGGATCCCCT TCCCTGGATA GTTCCAGTTT
  75541 ACATTTTCCA GTGAGAGAGA CTTGCATAAG AAATGGGGCC CTGGAGAGAT GGCGGGACAG
  75601 ACCTGTACCC ATCAGTGGTG GCTGCAGGTG GAGGAGCAGG CAGATGTCAG GTTCTCAGTG
  75661 GCTTCCCTGC TAGGCTAGAG ACCCACCTGC TTTGCTTGTG CGGACTAAGA TGAGTGATAA
  75721 GAGCTTCCTC ATAGGTTCTG GAGAATATAG CAATCTCGCA GCAGGATTCT GAGAACCTCC
  75781 CATCCATGCT TCAAGCTGAA GTCTTCAGTA TATGOTTCCC TGACCTCCCT ACTGCAGCTT
  75841 CCAGGAGCTC CAATGGTGAC TGGTATTATT ATAGTATCCC ACTGCTGCTA TAACAAACTA
  75901 CCACAACTAT GTGGCTTAAA ACAACACCAA TGTATTCTCT TATAGTTCTG GAGGTAAGAA
  75961 GTCAAAAATG GGTCACTATG GGCTAAAATT AACATGTCGT CAGAGCTGCA TTTCTTCTGA
  76021 GGCTCTAAAG GAAAATCTGT TTCCTCGCCC TTTCTAGCTT CTTGGGGCTG CCCGCATTCT
  76081 TTGGCTTGTG GTCTCTTCTT CCATCTTCCA AGTCAGCAGT CACATCACTT TGACCTCTGA
  76141 TTCTGTTATC ACAGCTCTAA CTCTGATGCT GCTTTCTACT CCTTTCACTT ATAAGGACTA
  76201 TTGTGATTAC ATCGAACCTG CCCAGATAAT CCAGGATAAT CTTCCCATCT CAAGAGCCTC
  76261 AGCTTAATCA CATCTGAAAA TTTCCTTTTG CCCTATTAGG AGACATTTTT ATAAGTTCCA
  76321 GGGATTAGGA CACAGACATA TTTAGGAGAC CGTTATTCTG CCTGTCACAT GACTTCACCA
  76381 ATATTTGCTA CCCGTGGTTT CCAAACATTA GTGTATGCTC TTATTTCTAT TTATTCCTGA
  76441 AATACCAGAG TGAGTCTGTT TTTCTGAAAA CAGCTCACTA ATGCCCAGAG GAGCTCAAAA
  76501 ATTTTCTGTC TGTTGTCTCA AAGTCTCCAA ATTGTACTGA TTCTTCAATG TGCATGGCTG
  76561 TAAATTAGGC AATAATCTTG CTGGGGTGTT GGAAGGAGAA GAAAGGGAAT GATTAGACAG
  76621 CCCTTCTGAT TCGGGACAGA ATCTGATTCG GGACACTGTC CCTGATGGCT GGCCATCTTG
  76681 GGCTGTAATC CAGTTTCCCT CCCACGTAGA CCCGGTCATT TCTGACTCCC CAAGAGGTTT
  76741 TCACAGTATT CATGTCCACT TCCTTCATCA GTCATCTTCT TGTCTTATAT CTCCATGTTT
  76801 ACATTTTTCT TCTCTTCTAC CACTGCAGTT CTGCCTTGAT TCTTCAGCCA TTGCCTTCCT
  76861 TTTGCATGCA TGTGCTTCAG CCAGTTCTCT AGGAAGACAT TCCAGACAGC TCATTATCAC
  76921 TTGTTACTAT GTTTATTTGA CTGCAGAGCT TTCAAGATTT GTAACTTTCT ATTTTTTAAA
  76981 TTTTAGTGTA AAAATAATTC TTGCTCACAG TAGGAAATGA AACAATCAAA ATATGCATAA
  77041 ATATACTTTT AAATCTCTCC TTTCACTAAA TCCAAACCCA CTTAACTAAG GTGATTACTA
  77101 TTGTATCAGC CTACTCTCTG CATGTCTACT TTCTTCACTT GCACGCAAAC CATAGTGAGT
  77161 GTTTTTATCT GGTAGCTTTT GTTGAGCTGT AATGACTATA AGTTTTATAT GTAATTTAAT
  77221 CATTTTCAGG GCAACTTGAG ATATAATTTA AATAGTATAA AATCCGCACA TTCAAAATAT
  77281 ACAATTCAGT GGCTTTTAGA ATATTCACAG TTGTGCCTCC ATTACCACAA TCAATTACAG
  77341 ATCCTTTTAT CTCCTCCCAG CCCTGCAGAA AAATCCATAT CCAAACCCCA GCCCTAAGAA
  77401 ACTACCAACC TATGTTTTAA CTCTACAGAT TTACCTATTC TGAACATTTT ATGTAATGAA
  77461 ATTATACAAC ATGTGGCTTT TTGTGTCTGT CTTCTTTTAC TTACCAGAAC ATTTTCAAAG
  77521 TTTGTGTGGG TCGTAGCTTG TATCAGTACT TCACATCTTT TTCATCGCTG AATTACATTC
  77581 CATTGTATGG ATATATCACA ATTTATTTAT TAGTTGGTGA CATTTTGGTC CTTCTCATTT
  77641 GGATTGTGAT TAATAATGCT GCTATAAACA TGGCATATAG GTTTTTATGT GAACTCATGT
  77701 TTTCATTTCT CTTGGGTATA CTGTATCATA GGAGGGGATT GCTGGGTCAT GTAATAACTT
  77761 AATGTTCACA AATTTGAGCT ACTGCTAGCC TGCTTTCTAA AGGGGCTATG TCTCTTAAAT
  77821 CCCCACCAGC AAAGTGTGAG TATTCTAGTA TCTCCACATT CTCATCAACA CTAATTATTA
  77881 TCTTTTTAAT TATAGCCATT CTAGTGGGTG GGAAATAGTA TCTCATTTTT GGGTTGATGT
  77941 GCATTTCCCT GATGGTTAAT GATGTTGAAC ATGTTTCTAT GTGCTTATGG GCCTTTTGTA
  78001 TATTATTTGT CTTATTATTA TTATTATTAT TATTATTATT ATTTATTGTT TATTATTATC
  78061 TTATTATTAA TATTATTATA TTGAGTTACA ACCATTOTTT ATATATTCTA GATACAAGTC
  78121 CTTTCCTTGC CAGATATAAG ATTTGCAAAA CTTTTCTCCC ATTCAATGAG TTTCTTTTCA
  78181 CTTTCTTGAT GGTTTCCTTA GACTCACACA TTTTTAATTT TGATGATGTT CAATTTATCT
  78241 TTTATTTCCT CTTTTTGCTT GTGCCTTTGG TATCATTTAA GATCAGGTAT TTTTACCTAA
  78301 TCCAAAGTCA TGAAGATTTA TGCTTATGTT TTCTTCTAAG AGTTCTCTAA TATTAAGTCT
  78361 CACATTTAGG TCTTTGATAC ACTGAGGTAA TTTTTATATG TGGTGTGAAG TAGGGATCCA
  78421 GCTTTATTCT TTTGCATAGG GATAGACTGT TGTTTCTGCA CTGTTTGATG AAAAGATAAT
  78481 TTCTTCCCCT CACTGAATTA TATTAGCACT GTTTTATAAA ACCAATTAAC CATATAAGTG
  78541 AAGAGTATTG CTGGGCCCTC AATTTTCTTC TGACTTCATT CTTTTTAATG CTGTTGTAAG
  78601 TTGGATTTTT AAATAACATT TGTATTAATG CATTGATACT CTATAGAAGC ACACTCAATT
  78661 TTTGTATTTT GATCTTGTAG CCTACCAATG TGCTAAACTA ATTTAGTATT AGTGGTTTAG
  78721 TAGTTGCTAA GCTTGTTTTT AGTGAATTCT TTCGTATTTC TATATAAGAT TTTATCATCT
  78781 GCAATTGAGA TAGTTTTACC TCTTGCTTTT TAATCTAGAT GACTTTTATT TCATCTTATT
  78841 GCTCATTGTA GCTTTTGAAA AATTACATTG TATTCACAGT GCCCTACCAT TTGATATTTT
  78901 TATTTCTATT ATATCATGAA CATCCTTTGA ATTTCACAGA AAAAATGGTT TAACTCATCA
  78961 TTTTAATGCT GTTCCATAGT ATGTTATATG CACCTACTCC CCTGGGGTAA ATATTTAGGT
  79021 GGTTTCATTT ATTTGTCACC CACTCTACAG ATGTCTATAT GCACATATTC TCCCAGCCAT
  79081 CCCAGAGTTT TCTGATTTCC CATAACGTCA GTGATTAAAC TCCTGATCTG TCTGACTAGC
  79141 TAGACATGCT GACTGACTGA CAGTTCCATT GCTACTTTGC ATAATTTAAG ATCAACAGCT
  79201 ACATTTGGAT CTCAGATCAT CTAGGCCCAG CTGCAGTTAG TCTTTCCTAG TCTCAGAGAT
  79261 TCCCTCTCTG CCTGTTAATT TCTCTATCCC TGAAACTGCC CTTGTACTCC AGCCCAAGCA
  79321 GCTTCCTAGC CCAGTAGTCC TGGATCTGCC ACTTGCTAAC TAGAAGGTGT CATCTGCAGA
  79381 AGGTAAGTAA GGCATTTATC TCCATTCACT TTATATTTAG TTATCAAATC TCCATGTAGC
  79441 TCAGGTAGAT CGAGCTGAAA ACATTCTTGT GTTTAGTAAT ATAGCCATTG AGCACACCAT
  79501 CATTGCTCTT TTCATACCTT AGTCCTACTC AAAGATTAAA GTGACAGCTT CCCAGTTATG
  79561 ATAAAAAAGT ATAAAACATT CTGAGGGACC TGTAAACTAT AAAGCTAAAA TATCAGCTTC
  79621 TGCATGCAAC ATATATGTCA AATTCTATGT CCAATACTCT TCTCATTTTT AGAATTAATT
  79681 CTGCAAACTT GAAGATTGAG CTTTTTATGA ACTGATTGGC ATCTGCTATG CTCACCAGAT
  79741 TATTCCCACT TGACTGAGAG GATGGTGGTA GCAAGAGCTC CTTCCTTTCA GAGGCTTTTA
  79801 AAAATACTTA TTGGGAAGAA TGTACCACAT AAGGCATTTA ACTAATGAAA CCACAGATAT
  79861 AACCACTGGC AAAAAAATCA AACAAACATT TCTTTAACTG CCCTGTACTT GGTACATTCT
  79921 CTTCCTCCTG CTAGGGAAGT ATCTCCTCTC CCTTCACTTC ACAGACTGGA ATTTTCCAAA
  79981 TTCCTAGTCT CAACAAGACA AACTGTATTT TGAAATCACT GTTTTCCCAG AATACCCGGA
  80041 GTGTTACCAT GACTCCAGAA GATGCCTTCA ACCAAGACTC ATAATCTGAC TGTAAGAAAC
  80101 CTTCAGATAA GGTATTGGGA ACTTCTGTAA AATTGTAGCT GGGCTGTGAT TAGAGAATTT
  80161 TCACAATATC AACGAATCTT ACAAAAGAGC TGCTGATCCA ACAGGTCCTC AGAGATCCTG
  80221 TGTGGGAACC AGAAGGTATT CTGTGAAAAA CGACAATAAG AATAACCATA ATAACAATAA
  80281 TGGCTGCATT TAAAAGGCTC TTGCTATATT TCAGGCACTG GTTTTTGTAC CTACATGGAT
  80341 TATTTTGTTC AATATTTATC ACAACCATAT GAGAAGGATG TTATTATCCA CAATAAATAA
  80401 ATGAAATCTG TAAACTGTAA CTTGTTCAAG AAAATTAGGT ACAGAGATGT TAAGTTACTT
  80461 GCCCAAAGTT CCATAATTTT GAAGATATGA GGCTAGAATT CAGTTCCAGG CCATTTTACA
  80521 CTAGATCTAC TAATTTAGCC ACTGTATGAT TGTACCAAGA CTGACCTAAA GCTACCTCCC
  80581 CTTCCCCAAC AGGTTCTTTT ATGACAGTGA GTATAAAATA CAGGGCTAAT GACGTATTGG
  80641 GTGCAGAGAG GGGTTGGAAA GAATGTGGTA TTTCAGACTA TGCTTACTAT GCTTAAAAAT
  80701 CACAATTCCG TGGTGACTAG AGTTTATCTT AAACTTTCCT CCCTGGCATT CCATTGCATC
  80761 CAGTTGAGAG AGCACTCTAG CTCCCTACAT CCCAGAACAT GTAGATCTGT CCGTGGTTCT
  80821 GAAAGACACA CTGGCAGGTC CCGCCAACTA CATGAATGGC ATTCATATTG TGGAAAACTG
  80881 CCCACTAGTA AAAATGTACA GTTGAATTTG TACATTCGTT ACTTACAAGG TTGGAGAACG
  80941 TGAAGTACTG GACAATCCAA CAATAGCTTG GAAAGATACC ATAAAAAGAC CCGCAGAATA
  81001 TTCTTTTATA TGCATTATTC TGAAATGTAC AAAACAAAAC CAGAATGGGA CACATTATGA
  81061 GTGAGAAGTT TATCTTGAAA TTTTAATCAG AATATGAAGA AAAGAAATCC AACATTAGAC
  81121 CTGGAAAAAG ATAAAATAAT TTGTATTTCA TTTAACAATA TTTATAGATT CTCAATATAT
  81181 ACACTATCCT TTTAAACCTC AATTTATATT TAACCATCCA TTTACCTTTA TTTTTTGTTT
  81241 GTTTTTTTCT TTCACCTCAT ACTTTCTACC TGGGATATCT TTTTGTGACT AAAGTACATT
  81301 CTTTAGAACT TOTTGTACTT TACTAGGTGT GTTTGTGTTA AATTGTTTGT CTTCAAAATT
  81361 CATTGAGATT TAAATTTTAT TATTATATTT TCATGCTTAA GTGTTATGTG TTTGTCTCTT
  81421 CAAATTTTTC TGAAGATTAT TTATAGTTTT ATAGTCACTA AATGTATTTA ACAATCTGTT
  81481 CTTTAATCAT AGCCCATTTA GCAATTTTAT ATCCTATACA ATTATTCTGA TTTCTCAGTT
  81541 ATATGATTGT ATGATTCTGC TGTCTGTTGT ATTTTCTTGT TCTTTCACGG TGCTTTGTCT
  81601 CTTATGAACA TTGTTCTCTG TTGTTGTTCT TAACTGTTAA GTGCTCCTTT TGCTGGTGTA
  81661 ATGATTTTTT GGAATTTTCA GAGTGCCAAT GACAGTAGAA ATTTTCCAGA GAAGTTTTGC
  81721 TTGATTTTCC AAGGTTCCTT GGAGTCTGCA CTACTTTAAA ATAAATTCAT GGCTTGATGA
  81781 TTTTCAGACC ACCCAGGGAG TGTGAACTCG GGCTGCAAAT CCACTTCTCT TTAATCTCAG
  81841 CAGAAGGTTT TGCCCCTCTC CACTCAGCAC CAAGGTGACT TTCTAGGGAG ACCTTCAAGC
  81901 AAGGATGTTT TCCTTATACT GAGGAGCTGG CTTTGGGATC CTGGTGAAAT GAGGAGAACA
  81961 CTCTATTAAA TTTCTCACTC TGAGTGCTAC CTGAACTCTG TCTTCTATTC CAAATGCCTT
  82021 AGGAGGCCAT GAAAACTAAA CCTTAATTAT CCTACATTAA GTAAATAAGC TCAGCAAAAA
  82081 AAGTAAATGC ACAATTCCGT GATTTAGGAT TTGACAATTT TTATTAAAGT TTAAGCCTCT
  82141 CAATGCTTCT ACATTGTGTT TTATTTTTTC ACCATCCATT TTTGTTAGCA TGAGGGTAGG
  82201 TATGTTTACC TAAATGTGCC ATGTGAGTGG TTCAAATCTC CTCCCCAACG CTCCTTATCA
  82261 CAAACCTTAC CATCTTATTC CTTTAAAGTC ACTGACTATC TAAGCAAACT ACTAGCTACT
  82321 GCTGTGCATT GATTAGGACC CAACAGTAAG ACGTTTCTCC TTTTGGTAAA CTGCACTTCC
  82381 AGATGGTCTA ATTGAACATC TTCTCACTGG AAGTATTTTT ATTTCCATTA CTCTTAATGA
  82441 TGATTCCTGA AAGAGACTCT AATGCCCTGA CAGTTAACTC TGGGGTATCC AAGCATACTC
  82501 AAATTTTTTT CCTTCTACAG TCAATTGTCC TTAGGGAACT CCTCAAGACC TCAATAATGA
  82561 GACAAGGGAG AGTGGACTCT CCAGTGGTAA TAAGAACATT AGGAGTATAG TACAAATTAT
  82621 TAGGCAACTT ACCTGGCCTT ATGGAACCTG CTAAAGTCTG ATGCTGTGTA TACCATTTCC
  82681 CCTTGCAGAC TGAGGGCTGG TGAGCATGAG TGACCTGATA AAACTCAGGT CATAATGGGC
  82741 ATCTCAAGTC ACTTGGTCAT TTTCTGTATC ATGATCATAG AATTCAGAAT CTACCAGGGC
  82801 CCAGTAGTAA GCCAGAAGCT ATTACTCAAA TTCAGAAAAA GAATGGAACT TTTTCCCCAA
  82861 ACCCTGAAAC TTGGGCAATG AGTCCTATGA CTGCCACACA CAAACACACA CACACACACA
  82921 CACACACACA CACACACACA CACATACACT CAACAATATC CAAATAAGCA TTTAGGGCCA
  82981 AATGGCAGAT ATGCTTGACT TTTGCCTGGA TCAAAATGAC ATTTCCATTT ATTATGGAAT
  83041 GTTTCTGTAC TTGTACTAGC TCATAGGATT TTGGATTAGA GAAATACCCT TGCAGGATAT
  83101 GAAGTCATAC TTTTTCATTG GTGAAATCAT CTGTTCTCAT TAGAGTTTAA TAATATCATG
  83161 CAGTTATTTC TCAAAGAGCT TGTACTCCTT AGATTTGCTG GAATTCCTCA AGTTAAAAAG
  83221 ACCAGGGCCT CAATAATGGG AGGTCTCATT GCCAATAGCT ACAACTGGCT GTAGAATTTA
  83281 TTAAAAACAT GTGAGTGCAT TGTTTGGGGA AAGAATCAAG AACAAAAGCA GTGCTTGTTT
  83341 TGTACTTCAC TGAACTACTT CCATGGCCTC CATTCAACTT TAGCTGCAGT TTTGGTGACT
  83401 TGATTCACAG ACTGTCGGAA AGTTATGAGC TATGGAATGT GCTCTCTCCA ATACTAAAGA
  83461 GGCCAGTCAT ATATATGTAG GGTGTCCTTC TTTATCATTG TAGGTTGTAT GAAGTCAGTG
  83521 ATTCCCATAG AACCTTGGGG TCACAGATGC CAGGCCTAAT CTTCTTTTAT TATTCATTCA
  83581 ATCTTTATTT TTTCAAACAA TGGTCTCACT CTCTTGCCCA GGCTGGAGTC CAATGGTGCG
  83641 AACACAACTC ACTGCAGCCT CGACCGCCCC AGGCTTAGGT GATCCTCCCA CCTCAGCCTT
  83701 CCAAGTAGCC GGGACTACAG CTGTGTGCCA CCGCACCTGG CTAATTTTTG TGTTTTTTGT
  83761 AGAGATGGGT TTTTGTCATG TTACCCAGGC TTGTCTTGAA CTCCTGGGCT CAAGCAATCC
  83821 ACCCACCTTG GCCTCCCAAA GTTGTGGGAT TACAGACATG AGCCACTGCA CCCAGCTCTA
  83881 ATCTTATTTT AATTTTAGAA ATCTAACTTG ACAGTTTCTG GTGCCTGGAT CCATATTTGT
  83941 CTCAGAAGAT TGAATTGTGG ACTGTGACCT TCTCTCCCAA AAGTCTCTTT CAGGGTAGAA
  84001 CTTCTGGTTA TGGCAACATG AAGAGATAGA CCAGCAAGTT TTCCTCCAAC AGCAACATGA
  84061 AAACTGTACT AAATACTAAA AACAAACATT TGAAGTCACT GGAAAATTAA CAAAGGCAAG
  84121 CAATATGTGA TAAGAGCTTT CTTGTTTAAG ACTCCTTCTG TCTTAGGTAA GGACTGCAAG
  84181 TTGGTGGCCT TTCCTTCTTA CCTATGAGTG CTCTTCAGCC TCCCAGCAGA TAACTGCAGC
  84241 CCTAGTGGCT CAATATGGGC AGTTTATAGA GTTCAGAGCC CAAATCAGCT AGAAATGTAA
  84301 ATGAGAATGT TGAAAGAGAG CTGCAGAAGA GTTGAGATCA ACAATGTGAA TGCAAATTAT
  84361 GCTCATCCCT AGTTGAACAC TAAACTCTGC ATGGGTGTGG GCCACTTGGG AATCTGGTGA
  84421 AAAACCAGAA AGAAACTAGA GGGAGGTCTA CCCTCCAAAG AGATAAATGA TCCTGGTGAG
  84481 GTCTGCAAGT CTTCAGTAAC ATGGACTGAG TGCATTTGCC AACCTGCACA GAGCTCAGTA
  84541 GAAGAAAGCA AAAATCTTAC TGGCTTGAGA GGTGAAAAGG CAGGATTTGA GGCTAACAAA
  84601 ACAATTGGAA ATTCAAAGAG AATTCCAGAA ACAAAGCAAC CACAATGAAG TTGAATCACA
  84661 AAATCTGAGT ATAAATGACC CAAATACTTG GTCAACCTCT AAATTACACA GATGCAGGAC
  84721 ACACACCCAG AAACCCCTGC TGAAACCACG TAAGATACTA AGTGGAAATT CTCAAACAGA
  84781 AAAAAAAGAA AATGAAAGTA TTCTGGAAGA GGTCCACAGA AGTTTTGAGA TGGCAGAATA
  84841 AAGAACAAGC CTGTGCCTTT GAATATAAAT CTCCAGAATG TGCTCAATTC TAAAATCCAA
  84901 AACACAGATT GAAATAAGAT TAAAGAAAAA AATGAAGAGA ACGTCATGAA CCCATCAGAG
  84961 AATATAAAGC AGGTGACCAG ATATGCAAGT GGATCCTCGA AAGAAGACAC AGGATCAGAA
  85021 AAAAATATTT GAGAAAATAA TAGTTGAAAG TTTCCCACAT TTGGTGGGGG ATTTTAGCTC
  85081 ATATCTAAGG TGTTCAATAA ATGAAAGCAA AATAACTGCA GAGAAAATTA CAAATAGGGT
  85141 GACCAAGGCC TTGGCCTGGC CTGAGGCAAC TTAGCTAGCC CCAGGGATGT GTGTTCTGGT
  85201 GTGGAGTAAG ATCAGGGAGC TACCAGTGCA ATCTCTGCCT CTACATCAGC GGGACTGATC
  85261 ATTTTAATTT TGATTGCAGT TACTGCTGGT ATCTGAATCA AAGTGGTGAT GCTTCCTGAG
  85321 GAAATTTCTG AAGCCATTAG TCAATTAGAA CATTATAAGC CATGCCTCCC GTGAAGAGGA
  85381 GCATCTAATC CCAGCATTTG CACCAGGGAA CTCTGCCTGA AGGGATTACC AGTGACTTGA
  85441 AAGTGTAACC AACAACAATC TTGTCACTAT TACGCACCAG CTAAGCAATC TGAACATACA
  85501 TGCTGAAGAC ATATTTGGTG AGCTATTTAA GGAGGCTAAC TACTTCCACA TTAGATCAAA
  85561 TTCTCTTAGA ATTTGTTGCC TCATGGTCAA AGTCACTCAG CTGGATTCAG CAGTGGAAGA
  85621 GGTGTCACCA CAGGACATCA ACATGGAAAA AGCCCTCAAA AGTTCCCCAG CCCAAGACCA
  85681 GCAAGCAGTT TCAAAGAGCA GCATTCCTAC TCCTGCTGCT GACATTTACA ATCAGAGTGA
  85741 TTGACCACCT CCCCTGAATA GCATGAGAGC ACATGGAGAT GATAAAGACA GGCTGAAGTT
  85801 CTATACTGAT CCTTCCCATT TATTTGACCT CTGGAAAGAA GAAGTGCTGC AGGACACAGA
  85861 AGACAAAAGG AAAGACAGAA GGCATCAAAA GTAGCAAAAC CACATAGATG GCACCACTTA
  85921 TGATGTGAAA AGGATTACAA AAGCCAGAAA CAAGCACCAT GAGTGGAATA TGATGGCATA
  85981 TGACAAAGAG CTTAGACCCA GCAACAGGTT GTCTCAGTGT GTACCATGGA GCATCCTGCA
  86041 AGAACCCCTG TCCCTAGACA GCAGGTCCCA TACAATGGAT GTTAGGGATG CTCTTATCGA
  86101 GCTACTCCAA ACCATTCTCA CGACCCTTCC TAGGCAGCTG AAGATGCATG TCCACATGGG
  86161 CCCACAAACC AGGCCCCTGA GCTTGAGTGC CACCCCTCCT CTGCCTCAGT GAGGCACATG
  86221 GCCCTAAATG GACCTCAAGA GCGGCCCCCA CTTCCCTGCC TTACCCCAGC TGTGTTCCAA
  86281 TCCTGTGCAC TTATGGCTCC AGGAGACTAC AGGATGCTCC CAGCACAGGT GCTTGCATAT
  86341 TACAACCCGT CAAGACCTCC TTCTCTTCCC CCTTTTACAG TACAAACTGC CTTTGTTGCC
  86401 TTCTGCAAAT CCTCCTGCCG CCTCCTTCCT CATATCAGCT GGCTCCACAC ACCCTCCTCC
  86461 TCACCCTCCC TCCACTGGCC CCTGGGTCCA GCATTCCCTC CTCCAGCCAC ACACTTTCCC
  86521 CACCAGGCCT CTTGGCTCAG TTCTTCTTTT TTGTCTTCCC CATGCATTCG CCCCCACACT
  86581 GCCACCAGTA GCTGAGGCAA AGTGTCAAGA ACCTGCACAG CCACCAAAAA GTGATACGAG
  86641 AAGTGATCTC CTTGCTGCTA CTGGAATGGG GATTTGACTG GAAAAGGTGC AGGATCTGTG
  86701 TGAGCAAGAG GCCAAGAGTG AGCCCCTTGG GAAGGACAAG GTCACAACCC TGTCAATACA
  86761 TTGCAGTGGA GAAGTACAGG GATTCTGAGG GAGACTTAGA GTTTGACAAA AACAACTGGC
  86821 CCAACCAAGG CAGTGCTGGG GCTAGCTCTA TGCAACAGGT GGGTAGCTGG TACCAGGCTG
  86881 GGTGACAGCC AGGAGACCAT ATGCTAAGAA TGTACTTAAG ATTTCAAGTG GTCTCAGCAC
  86941 TCAATGGTAT TATAATCCCG CAGCTGAGCA CCTTTTGTAT TAATGATGTG ATATTGCTTC
  87001 TGCACATCCA AAAATTCTGG GCTTTCTCAG TATTTACTGT GTAACATTTA AATGCCATTA
  87061 AGCATAGAAT ATCATGCTGC ACATGAAGAA ATAGGATGTA ACTATTACAT CGTCCTGAAA
  87121 AGAGCGTTTT TTTTTTTCCT GTAGGCCATG AAAGTATTTA GTGTATTTTG GGTTTACTCT
  87181 TATTGATCAT TACAATTCTG CAGATGTGCT GTGTGAAGCT GCCTGGTGCT AGGCCTTTGA
  87241 AGATTAATAA CTGGGTTTCA GGTGGATATC TTCAGATGAG TAATTGATAT TGTTTTACTG
  87301 TTTTGGGGTG TTTTATGAGT TTGAAAAGCT GTTGGTTCTA GAGGGAAAGG TTTAACAAAT
  87361 AAGGAGAATC CAATATTTCT GCTTTCATTT TCTTGGCGTG GTATCCTCTG TGTGAATTTG
  87421 AATGCTCAGT TGAATAGTTT TATTGCCTTA GTACACACTT GTCAAATGTA AAGTTGGAAG
  87481 GACCCTTTTC AGGAAGGGTT TCCTGTGAAC ATTCACTTAG TAAATGCTTG TGGATGATTC
  87541 TTGTAAGAGG TCTCAGTGCT TAGAAGTAAT GAAAATGAAG AACAAGATCC TTTCACCTTC
  87601 AGGGGACTTT GGGACCAATA CTCAAAATAT TTGCCAGCCC TTACAGCTTG GGCACCAGCC
  87661 AGCAGGAGCA GAGGTGAGTG GTCCAGGAGC TGGGCAGCTC TGGGCAGAGC AGCTGCATGT
  87721 GGGCCCTGGG TGCTAAAGCA GGCCTGCTCA TTGTGTGTCC TGGAAGAGCT GTGTTCTAAA
  87781 GGAGGCCACA GGAGTTCTTA GCTCCTTAGT GTGTGACAGA CAGTGCTTCA GAGGGCACTG
  87841 TGCTCCACTT GGTCTTAATT TTGAAAGTTT ACATTGAGGT GAAAGTAGAT ATTGACAGCT
  87901 GTTCACTCTT CATGTGCTGT GTTCAGATTA CGGATGAATC CATGAGCAGG GCCTTTGCCT
  87961 TATCCTAGAA ACACGTGGTT ATCTTCCCTC ATGTTGTTGC TGGCACATCA CATGTTGATA
  88021 AGTACCTCTC AAGATTACCT TGATAGACCC GTAGCTGTAG AAGAGCACTT AGTTCTCACC
  88081 TCTGCCCTTC CCCTTATTCT TGCACCGTGG TCATGCTAGA GACCCATGGG GTTTATTTTC
  88141 CTGGCTAAGG CAAACTGTTG GGAAATGCAA GAATGAGAGC TTCTATTGTG TAATTCTGGA
  88201 TGAAGAATAA CTGCTTGTTT AGAACAAATA AATGTCCCAG GCTTTGATTA TAGTTATGAC
  88261 TTCAAAAATA TGCTAAGACC ATTTACTTAC TGAGGAAATC TTACCTACTT ATAGTATTTC
  88321 AAATTGATTG CAGTCATTAG TGACTTTCAG TTACGATGAA GGGAAGAATC TTGAGCTAAA
  88381 CGCAGGGTTG TCTGAGGTGA TGATAGGAGT GGAAACAGGA TGGTAACTAA TGGATTTAGG
  88441 TTAATGAGTT CTGAGAAAAT CAACCAGCCC CTGCAAGTCA CAGCTCTGTG TCTCTAAGTG
  88501 GACACCTGTC TGTCTTGAGG CAGTCATCAT ATAATACGCA TCACATAGAT TTTAGTCATA
  88561 TGGATTTTGA AACTCTGGAA ACCATATGTC TGCAGCTCCC TTTCATGACC ATGCTTGTGC
  88621 TGGTTTATCT GATCTCCTCA GCTGTACCAG CTGCTGTGGC ATGAGCTTCT TGAGCTTCTC
  88681 TTTTTAGAGC TGATCATTAT CTGGCGGCTG TCGCTATAAT TTTCTTGAAT ACAAGTAAAC
  88741 ATTATTTATA AACTTTTAAA TTTCTCTGTT TAATTTTTCT CTCTCTTTTT TTTTTAATGT
  88801 AGAGGCCTAC TTTGCTGGCA TCTTTGGGTC CTACTCAGTC ATAAACCACC CTCCCCACCC
  88861 TAGAGTAGCC ACTCTTGATT TCTAATACAG ATGAGTTCTG CCAGGTCTAG TTGAATGGCA
  88921 TTATACAGTC ACCATCATGA CCCTGGGTCT CATCATCTCG CTGTGTGTGG TCGTAGCCTG
  88981 TTCACTCCCA TAGCTGTCCA ATGTTCCACT TTATAAATAA CTCACATTTC TTGTCCTTTC
  89041 ACTGTTGATA GACAATTTTG TTGTATACAG ATTTTACAAA TCGTGCTGCT ATGAATAATA
  89101 TTTTCATATA GTTTGGCACA CACATACATG CATTTCTATT GGTTTAGAAC TAAGAGTGCT
  89161 ATTGCTGGAT CATAGGTGAG TAGATGATTG GCTTAGTCTC TCAAAAACCA AGTTTCTAGT
  89221 AGTAAACAAA CAGTAGAGGA GGGTGAATCT CTTACTTAGA AATTATTCAG ATAATAAATG
  89281 AAGAAGGAAT GAGAGACTGA GACTATAATA ACTCTTTTGC AACCCATCAT GAATTAACAG
  89341 ATTTAGCCAT TGAATAGCAA TGGCAATTAA CAACACAATA AAAGAAAAAA CTAATGTGAG
  89401 GTTCTTCCTC TTGATGGAAG AACACAATAC AGCACCATCA AACAAGTCCA AGTGAAAAAG
  89461 AAACCAAGCC TGAATAGAAG CTGATCTCCA TAGCCAATTA CCAATTTAGA GGCAATACAG
  89521 ATAATAGAAA TACATATTAA ACTATGCOTT GGGGAGCAAT CAGCAAAATG CACACTATGG
  89581 GAAGCTCTAC CACACAATAT AAATTTCAAG GAAGAATCTA TAGAATAAAG ACAACAAAAG
  89641 CATATTTCTA AAGGTAAAAC TAAATCATAA TATTGGATGA TAAAAATAAA AATAAAACAA
  89701 AGAAGTGATG ATCGTAAAAG TCAGGTTGTG TTTTTATTTG AGGGAAGAAA GAGTTTATTG
  89761 CTGGGCTGGA GCAAATGATG GGGCTTCTGG TTTGCCCCAC AAACATCTAT CCCTGGTGAT
  89821 TAATGGGTGT TTACTTTGTA TTAAAGGACA CAAATTTTAG ACATTTTTTT CTTTTGGAGT
  89881 CCTATATTTT ATTTTATGAC ACAAAGGCTA ATGAAAAAAT AATTTATAGA GTATGGTTAC
  89941 TGTTTTGCAC AAAGCCTCCT CCCCATCTTC TTTTCTGGAC ACTGAGCACC CAGAACAACC
  90001 AGCAGCCCCA GGACCCCCAG CAGGGGTGAC CTCACTTCTA GGTGTGCATG CAGCTTACAT
  90061 CACCAGAGGT AACATGCAGG TCTACTCCTT GACTCCTGGA AGAACCTGAT AGGACACAGC
  90121 TGAAGGGAAG CCTTCTCCGC CATCTGTAGG CTCTTGGCCA TCAGTGTAGA GGGAGCGGGT
  90181 CCTCACTTCT CCACAGGCGT CTTCATGGCC ATAGCCTCCT CTCTGCATGG GGAGTAAGGC
  90241 CTGGCCCTTC GCCTGAACAC GGTGACAGGG ATCTCGCCAA AGGTGGAGAT CACACCATTC
  90301 CTCCTTTAGC AACCTCACGC TTCTTTTGCT TTACAGGAAA GTTGACTCAG GCTGGTGTCC
  90361 AGTAAAGAAA TCCCAAGGAG AGGCCGGGTG CGGTGGCTCA CGCCTGTAAT CCCAGGACTT
  90421 TGGGAGGTCG AGGCGGGCGG ATCACGAGGT CAGGAGATTG AGACCATCCT GGCTAACACG
  90481 GTGAAACCCC GTCTCTACTA AAAAATACAA AACATTAGCA GGGCGTGGTG GCGGGCACCT
  90541 GTAGTCCCAG CTACTCGGAA GGCTGAGCCA GAATGGCGTG AACCCGGGAG GCGGAGCTTG
  90601 CAGTGCTCCT GCTCCCAGGA GGCGGATGCC CAGGATGAAC TTTAAGGTGG AGAGAGGAAT
  90661 GTCATTGCTC ATCCTCCAGG TTCCAAGTAA GAACCTTCCT GCCCAGCCCA TCTCGATCTC
  90721 CCTGAATCCT CAACGCTAAT AAGGACTGTC TCCTTCTCAC CTCCCTAGAC TGGGCTTTTT
  90781 AACACTGGAG AGTGGATGTG ATTTCTAGTT TCAGCATGTC CTGGTTTATT GTGCTGCCAG
  90841 TAAAATAAAA TCAAAATACA CATTGAATAA ATAATAAATA ACCCATAGTG AGCAAAGGCT
  90901 TACAATGTAT TTTTTTGAGT CAAGACCTCA CTCTGGCCTG GGCTGGAGCC CAGTGGTGGG
  90961 ATCGTAGCTC AATGCAGCCT TGTATTTATG AGCTCAAGGG ATCTTCCCAC CTCAGCCTCC
  91021 ACAATACCTG GAAATACAAG CGCCCACCAC CAGGCCCAGC TGTTTACTTA TTTTTATTTT
  91081 TTTAGAGACG AGGGTCTCAC TATGTTGTCC AAGCTGGTCT CTAATTCCTG ATCTCAAGCA
  91141 ATCCTCTTGC CTTAGCATCC TGAATTGCTG GGATTACAGG CATGAGCTGT CATGCCCAGC
  91201 TTGTAATGTA CTGACACTAT CAAGGTTTCT TTCTCCAAGT GTGGACACAG CAGCAGACAC
  91261 CCCTTGTCTC TTGGGTCAGG ACACTGGGTA GAGTGGAAGA GCAGGGCAAC AAAGTCACTG
  91321 CAGAAAGCAC CCACGTGGAA GAGGTCCAGC AGGGAGAGCC ACCTGTTCCA GGGACACCAT
  91381 ATTTAGGGAT AACTCCTCTT TCTGGGCAGG ACTGTTCTTT GATTACTTTT GTATTCACAA
  91441 TAGTTCTGAA ATCGTAGGAT GATGAGACTC AAGACTGGCT AGGGTTTTAT TTTGGTTTAG
  91501 TTTTTATTTT GTTTTGTTTT GTTTTTTTCA AAAAACCATC ATGAGATTTG TTAATCTTTT
  91561 GAATGGTTTT TGTGTCTCAA TTTCCTTCAG AACTTAACTC TTCTTAACAT TTCACAGTCC
  91621 ACTTTCAGTT AAAAATGTAC TAGTACCCAT AAATAGAGAA ATCTTGCAAC CATTTATCCT
  91681 CTATAAAATG TCCATCTTTA TGGTACAGAT ATTATATGTA ATATATCTAT ATAGGTTATA
  91741 AGTCCCACAA GAAAAAAACT TCTTCTTATC TTTAAACAGT TCTATGTATA TAAATTAGAG
  91801 TAAATGAGGG GAACAAGATA GCCTTTTGCA CTTAGCTCAG TATTTACCAT TTTTGATCAT
  91861 CTTCATCTTT TCTGATAACC TCATTTCCCA CCTGGTATCA TTTGCCTTTA GCCTGAAGAA
  91921 TTTTCTTTAC CAATTGTTAT TGTGCAGATC TGCTGGTGAC AAATTCTCTT AATTTTCTTT
  91981 TACCTGAAAC ATCTCCTTAT TTTGCCTGTC CTTGAAGGAC ATCTGTGCTG GATATAGAAT
  92041 TCTTAGTTGT CCTTTTTTTC TTCCAGCACT TTAAAGGTGT TATTCTACTT TCTTTTGTTT
  92101 CTATGGTTTC TAATAAGAAG TCATTGGTCA GGACAAGAGG AACAGAGAAA CCAAAACTGA
  92161 GGAAATAACA TGGTAGACCT AAAACTTATC AATAATGAAA AAAAAACCTA TATGAATCAA
  92221 CACTCCAATG AAGGGCAGAG ATTGTGTGAA TTGGTTAAAA AAAAAAACAA AACACAGAAC
  92281 CCAACCATAT GCTCTTTCCA GATATGCAGT TTCTTTGTTT CTTTTTTTTT TAATTTAACT
  92341 TTTATTTTTA GTTAAGGAGT ACATGTACAG GTTTGTTATA TAGGTAAACT TGTGTCATGC
  92401 GGTTTTATTG TACAGATTAT TTCTGTACCC ATTAGTACCC ACCCAAGTAT TAAGCCTTAG
  92461 TACCCATTAG CTATTTTTCC TGATCCTCTC CCTCCTCCTC TACCCTCAAG TGGACCCCAG
  92521 TGTGTGTTGT TCCCTTCTGC GTGTCCATGT GTTCTCATCA TATAACTCCT ACTTATAAGT
  92581 GAGAACATGT GGTAATTGGT TTTCTGTTCC TGCCTCAGTT TGCTAAGGAT AATGGCCTCC
  92641 AACTCCATCC ATGTTCCTGC AAAGGACATG ATCTCATTCT TTTTTATGGC TGCATGGTAT
  92701 TCTGTGGTGT ATATGTGCCA CATTTTCTTT TTCTTTTTTT TTTAATGGAA AATAACTTTT
  92761 ATTGAGATCC CACCAGTTGC AATATCTGTT CCCAGCATTA AGCTCCTTCT TCCTTTGCAA
  92821 TTCGGTCTTT CTTGAGTGGT CCCATGAATG CTTTCTTCTC CTCCGTGGTC TGGAAGCGGC
  92881 CATGGCCAAA CTTGGAGGGG GTGTCAATGA ACTTAAGGTC AATCTTCTCC AGAGCCCGCT
  92941 GCTTTGTCTG CACCAGCAAG GACTTGTGGA GGGTGAGCAC CCACTTCTTG GTTCCCACCA
  93001 CACAGCCTTT CAGCATGACA AAGTCATTGG TCACTTCACC ATAGTGGACG AAGCCACCCA
  93061 AAGGGTTGAT GCTCTTGTCA GACAGGTCAT AGTCAGTGGA GGCATTGTTC TTGATCAGCT
  93121 TGCCATCCTT GATAAGGTAG CCCTAGCCAA TCCTATAGAT CTTGTTGATC TCAGTGCGGT
  93181 GATGGTAGCC TTTCTGCCCA CCACGTACCA CAGAGAAGCC CACACGAGCA GGATGCCATG
  93241 CCCCAATACA GGCCACCTTG CACAGGCCTT GGTGGGTCTT GCGGGGCAGC TTCTTGGTGT
  93301 GCCAACGACT GGTGACCCTT TTGTAGCCTT TGCCCTTGGT CACCCTGATG ACGTCGATCA
  93361 TCTCATCCTG CCCAAACACT TGGTTCACAG GTACCTGGTG CTTGAGCCTC TCGCCAGCCC
  93421 AGTCCAGCTT CTCAGCCACA GTGCCTCCAT TCACCTGGAT CTCCATCAGG TGGGCCTTCT
  93481 TCTGGCACAG AGGAAGCAGG TGCATCTGGG TGTGGGCAAT GACGCAGATG ACTTGGCAGT
  93541 ACTTCTTCAT GCTGCTGAAG TCCTTCTCCA GCTGCTTCTT GCCATCCTCA TCCTGCCATT
  93601 TCTTGCAGTA CTTGGTAAAG GCCTTCTTCT TAGATTTATG CCAGTTCTTA TAGAAACGCC
  93661 TCTTGCATTC ATCACTGATG TGCTCAGCGA AGACAGTCTT GCAAGTCCGG AAGCCTTGAG
  93721 GGGTTTCCAC GCAGCCCACA ATGCCCACTG GTGGCCTCTC CACAATGGTC ACAGCCTCCA
  93781 CCACCTCCTT CTTGTTCACC TTGGATCCTG GCCTGTCGAC TTCCCGCACG ATGTGGGTCA
  93841 TGCCAGCCTT GTATCCCAGG AAGGCTGTGA GGTGGACCGG CTTGGATGGG TCATCCTTAG
  93901 GGAAGCTCTT CACCTTCCCA CGATGCCTGC TGCTGCGCTT CCAAGGCAGG AAGCCGAGGG
  93961 ACCCATGTCT GGGAGTGGAG AACTTTCTGT GAGACATCAC GCCATCAAAT CCTGCCGGTA
  94021 GAGCATATTT GCCACATTTT CTTTATCCAG TCTACCACTG ATGGACATTT AGGTTGATTC
  94081 CATGTCTTTG CTATTGTGAA TAGTGCTGCA ATGAACATAT GCATACGTGT AAGACATGGG
  94141 CTTTCTTTTT TTAATTTGCT TTGTTTTATT TTACTTTAGG TTCCAGGATA CATGTGCAGA
  94201 ACCTGCAGGT TTGTTACATA CGTATAGTCT GCCATGGTGG TTTGCTGCAC CTATTGACCC
  94261 ATCCTCTAAG TTCCCGTCCC TTGCCCCCCA CACTCCCCAA AAGGCCCTGA TATGTGTTGT
  94321 TCCCCTCCCT GTGTCCATGT GTTGTCATTG TTCAACTCTC ACTTAGGAAT GAGAACATGA
  94381 GGTGCTTTGT TTTCTGTTCC CGTGTTAGTT TGCTGAGGAT GATGACTTCC AGCTTCATCC
  94441 ATGTCCCTGC AAAGGACATG AACTCATTCC TTTTTCTGGC TGTGTACTAT TCCATGGTGG
  94501 TATGCACCAC ATTTTCTTTA TCCATCTATC ACTGATGGAC ATTTGGGTTG GTTCCAAGTC
  94561 TTTGCTATTG TAAATAGTGC TGCAGTAAAC ATACGTGTGC ATGTGTCTTT ATAGTAGAAT
  94621 GATTTATATT CCTTTGGGTA GATACCCAAA GCGTCAAATA GTATTTCTGG TTCTCGATCT
  94681 CTGAGGAATG GCCACACTGT CTTCCACAAT GGTTGAACTA GTTTACATTC CCAACAACAG
  94741 TGTAAAAGTG TTCCTATTTC TCCACAGCCT CACCAGCATC TATTGTTTCT TGACTTTTTA
  94801 ATAATTACCA TTCTGACTGG CGGGAGATGG TATCTCATTG TGGCTTTGAT TTACATTTCT
  94861 CTAATGAGCA GTGATATTGA GCTTTTTTAA TTTTTCTCAT TAAGATTTTA ATGTCTCATT
  94921 TTAAAAAATT AAAGGAAGTT TCCATTTATT TTTTAAGATA AAGATTTAGC GCACCCAAAT
  94981 GCCCCCAAAG CCAACAGAAA AATAGCTTTG CCCTGTCATT TCCCTAAGAA AGCACTGCAG
  95041 TTACTCAAAA TATGCTCAAA GAAAAAAATG CAATCCTCTG AGTTCTAAGT TTCACGAAAG
  95101 GACCACATGT TAAACTATGT ATATCGATTT GATGTGTAAG TATGCAATAA ATATGTACAC
  95161 ATACATTCCT ATCTGCTTCA CATCATTCTA GAGTATTCAT AGTATATGAA GATGGGATTT
  95221 AGAAATGTGA AAAGGCTGTA ACAGTGAAAA GGAAAAAAAG GGTACAATGG TTTTTAGACC
  95281 AGTAGAATAA TAATGCTTAG CTAGTTAATT ATTTTAACTT TGGAGCAGAC TAAAAAAGTT
  95341 GTTTTGAAAT AAATGGTACC TGTAAGTGAT GOTTACTGCA ATACTTGTTA AGTGTTATTT
  95401 GTCACCACTG ACAGGAATTA TATAGTGCTA GTAACACAGC TCCCACTGTC CTAGAATTCC
  95461 TCTAAATGGC CAGCTATTGC TACCATTATC TTGATAAGCA TTTCAAGAAT TGTGTTGCTG
  95521 AGCCTTTTAA AAGATAACTA GTTTATAACC TATGTCTCCT TATGTGCCTA CATCAGTTAC
  95581 TCATGGTCCC AAAGAAGTAT AAAAGAGGAT ATAAGCTGCT GAAGATTTCA CAGAAGAAAC
  95641 AGAGATATCA GAAACTTCAT GATCATCAAA CTTAAACCAC CGCTGTTTTG CTGCATTTTT
  95701 ACAGTAGGCT GTGTAATGGC CTCCATCCAG CCCACCGCAG TGATCTGAAA CAGAAAACAA
  95761 ATTATATTTC TTCAAATTGT TCTTTGGACC AATAACATAC TGTGACAAGG CAAGATTTTC
  95821 TAACAGGAAG TCCACAGATG TCTGTAATTT TTGTTTCCAC CTGCCATTGT AGGAAAAATG
  95881 TTTCAGATGC ACTAAAAGCA CAGGTGGTAA CTTCCAGATT TCTTTTTTAA GAATCCCGTC
  95941 GAGCTCTGCA AAGATTGCAG TAAAATCTGT TATTATCTAT GAGTTTTTCT TCTTTAGAAA
  96001 ATAATCTAAG GCAATCCTAT AATGTACATT TACTTGTGGA TGCTATCAGT AGAGACAAAT
  96061 ACATGAAGGC CTCAAGTGTC CTAGACTTTT TGTGACGGGT GAGGCACTGT ACTGTAGATT
  96121 TGAATTGACC CTGAAAAAGT GCAACAATAA TAGACTCATA GAGCCGCTTG TGTTTCTGCC
  96181 AGGCATGTTC TGCAGCTTTA AAGTCATTGA GATGATCATT ATTTTCTTCT TTATATGTCT
  96241 TCCGATTATC AGTTTTATTT AGATCTTCAT GGAGACCATC CATTAGGAAC AGAAGAAATT
  96301 CTTGTGAATC TTGACTGTAT CCTGCAAACT GGTAATTGAT CTTCCCAATG GTGACTTTAA
  96361 GGTCTTTTGG ACTGATATAT CTATACTGTC CTGTCCACGG GGCTTTCATG ATTATACCAA
  96421 ATTCTTCTGC CACTTCACCT TTATGCCCCT AACAAATTTG ACTTGTTAAT ATCATCCTGA
  96481 TAACAGTTTC GGTTGAAATA ATCAGCCAAA TGTGGAGCAT TACATAGGCA CTGCAATATT
  96541 GAGTTCATAT AACAAGTATT TCCTAAGTTA CGAAGTCCAG TAAGAGCTGG TCCAGAACCT
  96601 CCAAAAACAG GATTGAGTTT CCAAATCTGA GAAGCAGAAA GCCTTGAGAT CTCAGCTTTA
  96661 GGGTAGCATG TTGGCTTGTC TTCCTGATTA ACTGTTGGAG TTACTGCTGG CTTCCTCTTG
  96721 AATAGCCTGG GTTATATCTG GGGAGGAGTA AGAGCATTTC AGTTTGGAAG GTTCCCTATC
  96781 CTGCTTAGCA GGAATCTGTG GCTTGCCTTT ATGAGTTGGA GGGGTGGAAG GAGGTGCAGA
  96841 TGAAGGAGCC ATTTCCAGTG GGTACATATG AACAGTGTTA GTGGTGAATG ATAATAATGA
  96901 AACGTTCCAG TGATTGGATC AAGAAACTTG GCCCAGCCTG AAGGCAGTCC TGGTACCATC
  96961 CTCCCCATTT CTTCACTTCG TGCTATTATC GAAGGTTCTC GTTGAGTTTT ATTTCTTTCA
  97021 GTATCATCTG TATCCTCTCT AAAAGTTTCT GTCCTTAGAA TTCCAGTTTC TGGTTGTCCT
  97081 TTAATCTTAA AAGGCTTGCC TGAACCTGAA TCCCCTGTCG CAGATGTATG GGACACATCT
  97141 CTTGTTGACT TTTTCTGTAC TTCTGGGGTT GAACATCTTC TCCCCCTGTC CTCTACAGAC
  97201 TTCTCGGCAC CAGAGGTTTC ATGATCACTT TTACTTTGTC TTTTTACTCT TGTTATTTCT
  97261 TTGCCCTTCT TTGCTGAGAT TTCTTTATCC TCTTTCTTGG CCTGTTCACG TTCTTTCTTC
  97321 TCCATTTCTT CTTTTGCTTT TTGTTGCTTC TGTGTAATTT CATTTTCTTC AGCTTCTTGT
  97381 TTCTTTTTGG CTTTTTGCTC GTGTTCTTCC CTCCTCAGTT TCTTTCTGTT TCCCTTGCTG
  97441 TCTTTCCTGA AGTTCTTTTT CTTGTTTGTT TTTCTCCATT AGAAGAGCAG TTTCTGCATG
  97501 AATATGAGCC TTTTCTTCAT CTGTTAACAT GAGAGTTGGA GAGGAAACTA CTGGCTTGGC
  97561 GGAACAATCA GGAATAACTT TTTCATTCTG AGGAGACTGT TGCTCATGAT TTGTACTTTC
  97621 AGATTTGATT ATATGCTCTT CAGGCAATTT GACTGCCAGT TTTTTAGTAT GATCAATCTG
  97681 TGGAACATTC TTTATGCTAG GCACTGGCTG AATGATGGGT GAAACATCAG ATTTAGAAGC
  97741 AGCAACTGAT TCAACTGGAA TTGATATATT CAGTGGTCCT GTCCTCTCAT TTTGATCATT
  97801 ATCACTTATT TGATCACTTA TCAATTCTAT GTCTTCATCC ACTTCTATAG GTGGAGGTGG
  97861 CATCTCGGCA GCAGGTTTAG AAGGAATTGA TTCTTCCAAT GAGGGATAAG TAAAATCCAA
  97921 TGAGATAGAC AACTCTTCAT TCTGGTGTTG TGGGGGTGGA GTGACCTTAG CATTTGTTGT
  97981 ATACTGGGAA TAGCAAAGGA ACCAGTTTTT ATAGCCTCCC TCTAAAACCA AAGGCCCAAG
  98041 GCCCATTGCA CAGGACAGTT TTACTTTCCC ACTTGAAAAG TGCATCTTTC AGATGCCAGA
  98101 GTGTTGTTCC AATCTGTAAA TCTTTTGCAG AACTAAACCA GTCAAGAAGT ACCATATACT
  98161 CCACATTCCC CCTCTTCTTC CATGTATCTA TAGAATCATC TGGGAGGTGT GCTTCAATCC
  98221 AGCTAGCAGT GACTCCTGGA CTGATGGCTT TTCCAGGAAC ACTGAGAGAA TGTAAAATAC
  98281 AGGAATCCTG ATAATCCTGC ATTCTTTGAG CATCCATTAT AATCAAGCTG ATGTTTTTAT
  98341 CCATCATCAT TGTGTATAGT TCCTTTGCTG TGATTGCTCC TTTCTCTTTG GTCTCACATT
  98401 TTTCATTCTT TTCACCATTG CTCTTTTGGG TTTTGTCTTT GGAATCCAAT ACAATCTCCA
  98461 AAGAATCTTT AGCCAACGTG CTGCCATCCT CTCTTCCTGT TTCCTGCCTT TTTTGTTGTA
  98521 GCCACTGTGC TTCCCCCTGC CTGTCCTTTT CCTCAAGTTT TTTCCAGATT TCAACTTCTT
  98581 CATATCTTAG TTTAAGGCTT TCAGAGAGTC GTTCAGTTTC TCCAGTGGCT TTTTTGATGT
  98641 TTGCAGGTCC AAGTATTGAA TGGAAGTAAT ACTGCTGTTG CTTGAAATCA GTTCTTTTTT
  98701 GGATAGGATT ATAAACAGTC ATGTTTCATA TACATGTGTG AGCTTTTTTT CATATGTTTC
  98761 TTGGCCACGT AAATGTCTTC CTCTGAGAAG TGTCTGTTTA TATCCTTTGC CCACTTTTTG
  98821 ATGGGGATGG GTTTTTTTCT TGTAAATTTG TTTAAGTTCC TTGTAAACAA ACATGTGAGC
  98881 TCTCATCATT CTTGTTTAAA CACCTAACAG TCATCCTAAC CAGTGCAACA AGGACCAGGC
  98941 ATGGTGGCTC ATGCCTGTAA TCCCTGCATT TTGGGAGGCT GAGGTGGGAG GATCACTTGA
  99001 GATCAGGAGT TTGAGACCAG CCTGATAAGT GAGACCTCAT CTCTACCAAA TAAAAATAAT
  99061 TTTAAAAGAA AATAGATCAA TGGATAAGAA AGGAAGAAAT GAAAGTCTTT CTTTGTCACC
  99121 AGCTTCATTG TATATGTAGA AAACACTAGG GAATTCTGAA AAAGTCTCTG GAATTAATCA
  99181 TTGAATTTGC AAAATAGTTC ATAAAATATA TGTAATAAGT CGCTTAGATG AACATGAAAA
  99241 GATAGCAAAC AATACTAGTC ATCAAAGAAG TGCAAGTTAA AACCACAATG AGAAACCATC
  99301 ACACATCACC TAGAGCAGGT AAAGTTAAAA AGACATATGA TAAGTCTTAA CACTGGCAAG
  99361 AATATGGAAA AAATAGGAAT GTCTGATATT GCTGGTAGGA ATGCAAAAAA TGTGGCAGCC
  99421 AATTTGTAAA GTGGTATGGC AGTTTCTTAT ACAGTTACCC ATCTATTACC ACATGGCCCA
  99481 GCAATTCCAC AAATACGTAT TTATCCAAAA GAAATAAAAA TGTAAGTCCA CACTTGTAAG
  99541 CAGTTATTTA TAGTGGCTTC ATTAATAACA AGCCCTAACT GGAGGAATCC ACATGTCTAT
  99601 CAACTGGAGA CACAGAAACC AATGAATAAA CTGGGATTCC AGCAATACTC GGCAGCTGCT
  99661 CAGCAACAAA AATGAATGAA TGGCATCATC TCAAACATCG TTATGCTAAG GGAGAGACCA
  99721 AACAAAAGAC TACATAACAT ATGATTGCAT GTCCATGAAA TTCTAGAAAT GTCACTATTG
  99781 CAGTGACAGA AAGCACAGCA GTGGTTGAAT GAAGGGAAGG GGGTGAGGGT GGGAGGCAAG
  99841 GATTAAATAG AAAGGGGCAT AAAGAAAGTT TTTAGGGAAA AGAAACTGTC CTCTACAGCG
  99901 CCACAACTCA GGAGTGACTG GGAGGGGGAA GTAAGGGGAG AAGAAGGTCT GAGGGATAAG
  99961 GGGCAGAGAG AAGGGCTGGG GAAGCAGGAG GAGAGGACGA GGAGCAGGGG AAAGGACTCT
100021 AAAGCAGTGG AGGGGCCTAG TAGGAGGATC TTTGCATTTG GTGTTTCTCT ACTGGGCAGT
100081 GTGGTAGTTA CACTATAAAT AATTACCAAT ATCCACCAAA AAGTGCAGCT AAAACTGGTG
100141 AATTTTTTTA CACGTAAACG CCCTGATAAG CAAAAAAAAA AAAAAAAAAA AAAAAAAAAA
100201 AAAAAAAAAA GGGGGGGGGG GAGGAAAGAA GGCAAAAATA AAGACATTGT TAGATCATCA
100261 AACCCATAAA ATTCATTTCC TAGGGGTCCT GTACTACATG TAATTTTAAA GGACGTTCTA
100321 CAGGCTGAAG GGAAATGATA CTAGATGGTG ACCCAGATAC ACAGAAAGGA ACAATTAACA
100381 ACAGAAATGA TGCACATACA CATATCACAT ACACACCCAT TTTCTTAATT TCCTGAAGAT
100441 ACGTGACTGC TTGTCTAAAA CAAAAAGTAT TACACTGTAT TGTTGAGTTT ATAACGTATA
100501 TTGATGTAAC ATATACAATA ATAATAGCAT AATGATAGTT TAAATGAAAC TACACCATTT
100561 GAAGTGTCCT TTATTTTGCT GGATGCAGCT TAATATTACC TGAATTTCAC TGTGAAAAGT
100621 CAAGGAATTG GGTTTCAATT CTTACAACAA TAAAAAATTA ATGTAAAGAA ATATAGCTAA
100681 AAGCCACTAG GAGAATTAAA ACCATAAGCT AAAAAATGTT TACTTGAAAC ATAAGAAAGT
100741 AGCAAAGGAG GAACAGAAAC AAAAAGATAT GAGACAAATT GAAAACGTAT AGCAAAATGG
100801 TAGACCAAAA TCCAACCATT ATAAGTGAAG AAATGACACG ACCTGAGTCA CATTAGCAGG
100861 ACTGCTGAGC ACTATGGGGA GAACAGACAT GGGCAGGAAG TGAGGGACAG TGTTAGTGCC
100921 ACAATTCAGG AGTGACAGGG TGGCGGGGAC TAAAGGGGAA AGAGGGTGTG AGGGATGAGA
100981 GGGGCAGACA GAAGGGCTGG AGAGGCAGGA GGTGAGGAAA AGGAGCAGAG GAAAGAATTC
101041 TAAAGCAGTA GAAGAGCCTG GCAGGGGGTT CTTTGCATTC GGTATTTAAT ACATTTTGTG
101101 TGACTGCCTT AAAACTAATG GGCTCCTTAT GATTTTTTTT TAAAAAGGGG TTACAAAAAT
101161 ATCAAGTGTC CAAATAAAAT ATGCACACTG CTTAGATGTG CATAGTTCCG GAAAACGGGC
101221 AGTGCTGGAG CGCTGGTGAA GAGCATTGGG ACTGCATGGA GCCCTCACAA CTTTGAGGTG
101281 ATGACTACAG GCTCCCGGTT GCAATAGACA GTAACAAACC CTGCTTCTTT GTATTCAGGA
101341 GATGTTCTGG ACTCACACAG GGAAACTCTG GCTAGAGAAT GAGGATAACT TTAAATGCAA
101401 CAACCCAGAG TCACAGATCC ATAGTCTGCG AAAGTAAAAC AGGAGCTTTG AGAATTTAAT
101461 TGTAATGCAG TTTTGACACA GGTCTTTCAC AGATTGGAAT TCTAATCATT CAGGGATTAC
101521 CAATATTGTG CTACCTACTG TATCAATAAA CAAAAAGGAA ACTGGTCTCT ATGAGAATCT
101581 CTACCTGGTG CTTTCAGACA AAACTTCACC AGGTTTAAAG AGAAAACTCC TGACTCTACA
101641 CGTCCATTCA CAGGGCGAGC TCACTGTCTG GCATCAAGTT CCCCATGGTG AGTTTCCCTG
101701 TACAAGAGTC CAAGGGGAGA GGTAAGTGTC CTTTATTTTG CTGGATGTAG TTTAATATTA
101761 CCTGAGGTAA GGTAAGGCAA AGAGTGGGAG GCAGGGAGTC CAGTTCAGGG ACGGGGATTC
101821 CAGGAGAAGT GAAGGGGAAG GGGCTGGGCG CAGCCTGGGG GTCTCTCCCT GGTTTCCACA
101881 GACAGATCCT TGGCCAGGAC TCAGGCACAC AGTGTGACAA AGATGOTTGG TGTAGGAGAA
101941 GAGGAATCAG GACGAAGTCC CAGGTCCCGG GCGGGGCTCT CAGGGTCTCA GGCTCCAAGG
102001 GCCGTGTCTG CAATGGGGAG GCGCCGCGTT GAGGATTCTC CACTCCCCTG AGTTTCACTT
102061 CTTCTCTCCC AACCTGCGTC GGGTCCTTCT TCCTGAATAC TCATGACGCG TCCCCAATTC
102121 CCACTCCCAT TGGGTGTCGG ATTCTAGAGA AGCCAATCAG CGTCTCCGCA GTCCCGGTTC
102181 TGAAGTCCCC AGTCACCCAC CCGGACTCAG ATTCTCCCCA GACGCCGAGA TGCGGGTCAT
102241 GGCGCCCCGA ACCCTCATCC TGCTGCTCTC GGGAGCCCTG GCCCTGACCG AGACCTGGGC
102301 CGGTGAGTGC GGGGTTGGGA GGGAATCGGC CTCTGCGGAG AGGAGCGAGG GGCCCGCCCG
102361 GCGAGGGCGC AGGACCCGGG GAGCAGCGCA GGGAGGAGGG TCGGGCGGGT CTCAGCCCCT
102421 CCTCGCCCCC AGGCTCCCAC TCCATGAGGT ATTTCTACAC CGCTGTGTCC CGGCCCGGCC
102481 GCGGGGAGCC CCACTTCATC GCAGTGGGCT ACGTGGACGA CACGCAGTTC GTGCGGTTCG
102541 ACAGCGACGC CGCGAGTCCG AGAGGGGAGC CGCGGGCGCC GTGGGTGGAG CAGGAGGGGC
102601 CGGAGTATTG GGACCGGGAG ACACAGAAGT ACAAGCGCCA GGCACAGACT GACCGAGTGA
102661 GCCTGCGGAA CCTGCGCGGC TACTACAACC AGAGCGAGGC CAGTGAGTGA CCCCGGCCCG
102721 GGGCGCAGGT CACGACCCCT CCTCATCCCC CACGGACGGC CCGGGTCGCC CCAAGTCTCC
102781 CGGTCTGAGA TCCACCCCGA GGCTGCGGAA CCCGCCCAGA CCCTCGACCG GAGAGAGCCC
102841 CAGTCACCTT TACCCGGTTT CATTTTCAGT TTAGGCCAAA ATCCCCGCGG GTTGGTCGGG
102901 GCGGGGCGGG GCTCGGGGGA CGGGGCTGAC CGCGGGGGCG GGGCCAGGGT CTCACATCAT
102961 CCAGAGGATG TATGGCTGCG ACGTGGGGCC CGACGGGCGC CTCCTCCGCG GGTATGACCA
103021 GTCCGCCTAC GACGGCAAGG ATTACATCGC CCTGAACGAG GATCTGCGCT CCTGGACCGC
103081 CGCGGACACG GCGGCTCAGA TCACCCAGCG CAAGTGGGAG GCGGCCCGTG AGGCGGAGCA
103141 GCTGAGAGCC TACCTGGAGG GCCTGTGCGT GGAGTGGCTC CGCAGATACC TGAAGAATGG
103201 GAAGGAGACG CTGCAGCGCG CGGGTACCAG GGGCAGTGGG GAGCCTTCCC CATCTCCTGT
103261 AGATCTCCCG GGATGGCCTC CCACGAGGAG GGGAGGAAAA TGGGATCAGC GCTAGAATAT
103321 CGCCCTCCCT TGAATGGAGA ATGGGATGAG TTTTCCTGAG TTTCCTCTGA GGGCCCCCTC
103381 TGCTCTCTAG GACAATTAAG GGATGAAGTC CTTGAGGAAA TGGAGGGGAA GACAGTCCCT
103441 AGAATACTGA TCAGGGGTCC CCTTTGACCA CTTTGACCAC TGCAGCAGCT GTGGTCAGGC
103501 TGCTGACCTT TCTCTCAGGC CTTGTTCTCT GCCTCACGCT CAATGTGTTT GAAGGTTTGA
103561 TTCCAGCTTT TCTGAGTCCT TCGGCCTCCA CTCAGGTCAG GACCAGAAGT CGCTGTTCCT
103621 CCCTCAGAGA CTAGAACTTT CCAATGAATA GGAGATTATC CCAGGTGCCT GTGTCCAGGC
103681 TGGCGTCTGG GTTCTGTGCC CCCTTCCCCA CCCCAGGTGT CCTGTCCGTT CTCAGGATGG
103741 TCACATGGGC GCTGTTGGAG TGTCGCAAGA GAGATACAAA GTGTCTGAAT TTTCTGACTC
103801 TTCCCGTCAG AACACCCAAA GACACACGTG ACCCACCATC CCGTCTCTGA CCATGAGGCC
103861 ACCCTGAGGT GCTGGGCCCT GGGCTTCTAC CCTGCGGAGA TCACACTGAC CTGGCAGTGG
103921 GATGGGGAGG ACCAAACTCA GGACACTGAG CTTGTGGAGA CCAGGCCAGC AGGAGATGGA
103981 ACCTTCCAGA AGTGGGCAGC TGTGGTGGTG CCTTCTGGAG AAGAGCAGAG ATACACGTGC
104041 CATGTGCAGC ACGAGGGGCT GCCGGAGCCC CTCACCCTGA GATGGGGTAA GGAGGGGGAT
104101 GAGGGGTGAT GTGTCTTCTC AGGGAAAGCA GAAGTCCTGG AGCCCTTCAG CCAGGTCAGG
104161 GCTGAGGCTT GGGGGTCAGG GCCCCTCACC TTCCCCTCCT TTCCCAGAGC CGTCTTCCCA
104221 GCCCACCATC CCCATCGTGG GCATCGTTGC TGGCCTGGCT GTCCTGGCTG TCCTAGCTGT
104281 CCTAGGAGCT GTGGTGGCTG TTGTGATGTG TAGGAGGAAG AGCTCAGGTA GGGAAGGGGT
104341 GAGGAGTGGG GTCTGGGTTT TCTTGTTCCA CTGGGAGTTT CAAGCCCCAG GTAGAAGTGT
104401 GCCCCACCTC GTTACTGGAA GCACCATCCA CACATGGGCC ATCCCAGCCT GGGACCCTGT
104461 GTGCCAGCAC TTACTCTGTT GTGAAGCACA TGACAATGAA GGACAGATGT ATCACCTTGA
104521 TGATTATGGT GTTGGGGTCC TTGATTCCAG CATTCATGAG TCAGGGGAAG GTCCCTGCTA
104581 AGGACAGACC TTAGGAGGGC AGTTGCTCCA GAACCCACAG CTGCTTTCCC CGTGTTTCCT
104641 GATCCTGCCC TGGGTCTGCA GTCATAGTTC TGGAAACTTC TCTTGGGTCC AAGACTAGGA
104701 GGTTCCCCTA AGATCGCATG GCCCTGACTC CTCCCTGTCC CCTCACAGGG CATTTTCTTC
104761 CCACAGGTGG AAAAGGAGGG AGCTGCTCTC AGGCTGCGTG TAAGTGATGG CGGTGGGCGT
104821 GTGGAGGAGC TCACCCACCC CATAATTCCT CTTGTCCCAC ATCTCCTGCG GGCTCTGACC
104881 AGGTCTTTTT TTTTGTTCTA CCCCAGCCAG CAACAGTGCC CAGGGCTCTG ATGAGTCTCT
104941 CATCGCTTGT AAAGGTGAGA TTCTGGGGAG CTGAAGTGGT CGGGGGTGGG GCAGAGGGAA
105001 AAGGCCTAGG TAATGGGGAT CCTTTGATTG GGACGTTTCG AATGTGTGGT GAGCTGTTCA
105061 GAGTGTGATC ACTTACCATG ACTGACCTGA ATTTGTTCAT GACTATTGTG TTCTGTAGCC
105121 TGAGACAGCT GCCTGTGTGG GACTGAGATG CAGGATTTCT TCACACCTCT CCTTTGTGAC
105181 TTCAAGAGCC TCTGGCATCT CTTTCTGCAA AGGCATCTGA ATGTGTCTGC GTTCCTGTTA
105241 GCATAATGTG AGGAGGTGGA CAGACAGCCC ACCCCCGTGT CCACCGTGAC CCCTGTCCCC
105301 ACACTGACCT GTGTTCCCTC CCCGATCATC TTTCCTGTTC CAGAGAAGTG GGCTGGATGT
105361 CTCCATCTCT GTCTCAACTT CATGGTGCGC TGAGCTGCAA CTTCTTACTT CCCTAATGAA
105421 GTTAGGAACC TGAATATAAA TTTGTTTTCT CAAATATTTG CTATGAAGGG TTGATGGATT
105481 AATTAAATAA GTCAATTCCT GGAAGTTGAG AGAGCAAATA AAGACCTGAG AACTTTCCAG
105541 AATCCGCATG TTCTCTGTGC TGAGTCTGTT GCAGGTGGGG GTGGGGAAGG CTGTGAGGAG
105601 CCGAGTGTGG ACGGGGGCCT GTGCCTAGTT GCTGTTCAGT TCTTCATGGG CTTTATGTAG
105661 TCAGTCCTTA GCTGGGTCAC CTTCACTGCT CCATTGTCCT TGTCCCTTCA GTGGAAACTT
105721 GTCCAGCAGG AGCTGTGACC ACAGAGGCTC ACACATCGCC CAGGGCAGCC CCTGCACACG
105781 GGAGTCCCTG TGCTTTCTGA GACAAATTTT CAGACCCATT CAGCTCCTGC CCTCCTTATA
105841 GGGCTCCTCT TCTGCTTTGG TCTCCTGCCC TCTCTCCCTT CCCTGATTCC AGTGATCTTC
105901 GTGCTGACTC CAATCCCAAC TCATGAATCT AAAGCAGAGC CTAATTTAGA TTTGTATTTG
105961 TTTGTAAAAT TGGGTCCATA GTCTAGAATT GTTCCTTCCT GAAGAGAGAA ACCTGATCGT
106021 GTGCTGCAGC GTGCAGGGCG GTTGGTGTGG GAGGAGGGAT AGGGGAGGGA GGACACACAA
106081 GCAGCCCTGC TGAGAAAAGT ACAGGCGGCC TCGGTGTCAG TGTGAGGGGA CCTTGTGCTG
106141 CAGCTGCCAC AAAACAGCAC TTGGCCTGAG GCTATGTTAA TAAAGATACT GGCTTTAGAA
106201 TAGGAGGTGC TCTACACTGA TCATTCAACT GACCTTTGTT GTCAGCCAGA CACAGGACAG
106261 AAAAGTTCTG CATCTGGGGA ACACCATTGA AGTAAAATCA GAAAAATATC TGAGCATATG
106321 CTCCAGTGGT AAGAGGCAGA CGATACATAC ACTATAACCA CAGTAAGAAA AGAAAGTGAT
106381 GGAAGGTGGT AAGTGCCATG AGGCAGGTGA TCCGGGTATG GGCAGTGGGG ACAGGGAAGG
106441 TGGCTGTTGG ACAGGAGTTG TCAATGTGTG CCTTGTTGCA AAGATGACCT TTGAGGAAAG
106501 ATTTGAGGGA CATGAGGATG TCTGGGGAAG TTCTTTCTAG GCAAGGAAAC ACAACTTCGT
106561 ATAGAAAGGT ATATACGAAG TTGTGCGGCC GCGTCGACCA ATTCTCATGT TTGACAGCTT
106621 ATCATCGAAT TTCTGCCATT CATCCGCTTA TTATCACTTA TTCAGGCGTA GCAACCAGGC
106681 GTTTAAGGGC ACCAATAACT GCCTTAAAAA AATTAGAAAA ACTCATCGAG CATCAAATGA
106741 AACTGCAATT TATTCATATC AGGATTATCA ATACCATATT TTTGAAAAAG CCGTTTCTGT
106801 AATGAAGGAG AAAACTCACC GAGGCAGTTC CATAGGATGG CAAGATCCTG GTATCGGTCT
106861 GCGATTCCGA CTCGTCCAAC ATCAATACAA CCTATTAATT TCCCCTCGTC AAAAATAAGG
106921 TTATCAAGTG AGAAATCACC ATGAGTGACG ACTGAATCCG GTGAGAATGG CAAAAGTTTA
106981 TGCATTTCTT TCCAGACTTG TTCAACAGGC CAGCCATTAC GCTCGTCATC AAAATCACTC
107041 GCATCAACCA AACCGTTATT CATTCGTGAT TGCGCCTGAG CGAGACGAAA TACGCGATCG
107101 CTGTTAAAAG GACAATTACA AACAGGAATC GAATGCAACC GGCGCAGGAA CACTGCCAGC
107161 GCATCAACAA TATTTTCACC TGAATCAGGA TATTCTTCTA ATACCTGGAA TGCTGTTTTT
107221 CCGGGGATCG CAGTGGTGAG TAACCATGCA TCATCAGGAG TACGGATAAA ATGCTTGATG
107281 GTCGGAAGAG GCATAAATTC CGTCAGCCAG TTTAGTCTGA CCATCTCATC TGTAACATCA
107341 TTGGCAACGC TACCTTTGCC ATGTTTCAGA AACAACTCTG GCGCATCGGG CTTCCCATAC
107401 AATCGATAGA TTGTCGCACC TGATTGCCCG ACATTATCGC GAGCCCATTT ATACCCATAT
107461 AAATCAGCAT CCATGTTGGA ATTTAATCGC GGCCTCGAGC AAGACGTTTC CCGTTGAATA
107521 TGGCTCATTT TAGCTTCCTT AGCTCCTGAA AATCTCGATA ACTCAAAAAA TACGCCCGGT
107581 AGTGATCTTA TTTCATTATG GTGAAAGTTG GAACCTCTTA CGTGCCGATC AACGTCTCAT
107641 TTTCGCCAAA AGTTGGCCCA GGGCTTCCCG GTATCAACAG GGACACCAGG ATTTATTTAT
107701 TCTGCGAAGT GATCTTCCGT CACAGGTATT TATTCGCGAT AAGCTCATGG AGCGGCGTAA
107761 CCGTCGCACA GGAAGGACAG AGAAAGCGCG GATCTGGGAA GTGACGGACA GAACGGTCAG
107821 GACCTGGATT GGGGAGGCGG TTGCCGCCGC TGCTGCTGAC GGTGTGACGT TCTCTGTTCC
107881 GGTCACACCA CATACGTTCC GCCATTCCTA TGCGATGCAC ATGCTGTATG CCGGTATACC
107941 GCTGAAAGTT CTGCAAAGCC TGATGGGACA TAAGTCCATC AGTTCAACGG AAGTCTACAC
108001 GAAGGTTTTT GCGCTGGATG TGGCTGCCCG GCACCGGGTG CAGTTTGCGA TGCCGGAGTC
108061 TGATGCGGTT GCGATGCTGA AACAATTATC CTGAGAATAA ATGCCTTGGC CTTTATATGG
108121 AAATGTGGAA CTGAGTGGAT ATGCTGTTTT TGTCTGTTAA ACAGAGAAGC TGGCTGTTAT
108181 CCACTGAGAA GCGAACGAAA CAGTCGGGAA AATCTCCCAT TATCGTAGAG ATCCGCATTA
108241 TTAATCTCAG GAGCCTGTGT AGCGTTTATA GGAAGTAGTG TTCTGTCATG ATGCCTGCAA
108301 GCGGTAACGA AAACGATTTG AATATGCCTT CAGGAACAAT AGAAATCTTC GTGCGGTGTT
108361 ACGTTGAAGT GGAGCGGATT ATGTCAGCAA TGGACAGAAC AACCTAATGA ACACAGAACC
108421 ATGATGTGGT CTGTCCTTTT ACAGCCAGTA GTGCTCGCCG CAGTCGAGCG ACAGGGCGAA
108481 GCCCTCGAGC TGGTTGCCCT CGCCGCTGGG CTGGCGGCCG TCTATGGCCC TGCAAACGCG
108541 CCAGAAACGC CGTCGAAGCC GTGTGCGAGA CACCGCGGCC GGCCGCCGGC GTTGTGGATA
108601 CCTCGCGGAA AACTTGGCCC TCACTGACAG ATGAGGGGCG GACGTTGACA CTTGAGGGGC
108661 CGACTCACCC GGCGCGGCGT TGACAGATGA GGGGCAGGCT CGATTTCGGC CGGCGACGTG
108721 GAGCTGGCCA GCCTCGCAAA TCGGCGAAAA CGCCTGATTT TACGCGAGTT TCCCACAGAT
108781 GATGTGGACA AGCCTGGGGA TAAGTGCCCT GCGGTATTGA CACTTGAGGG GCGCGACTAC
108841 TGACAGATGA GGGGCGCGAT CCTTGACACT TGAGGGGCAG AGTGCTGACA GATGAGGGGC
108901 GCACCTATTG ACATTTGAGG GGCTGTCCAC AGGCAGAAAA TCCAGCATTT GCAAGGGTTT
108961 CCGCCCGTTT TTCGGCCACC GCTAACCTGT CTTTTAACCT GCTTTTAAAC CAATATTTAT
109021 AAACCTTGTT TTTAACCAGG GCTGCGCCCT GTGCGCGTGA CCGCGCACGC CGAAGGGGGG
109081 TGCCCCCCCT TCTCGAACCC TCCCGGTCGA GTGAGCGAGG AAGCACCAGG GAACAGCACT
109141 TATATATTCT GOTTACACAC GATGCCTGAA AAAACTTCCC TTGGGGTTAT CCACTTATCC
109201 ACGGGGATAT TTTTATAATT ATTTTTTTTA TAGTTTTTAG ATCTTCTTTT TTAGAGCGCC
109261 TTGTAGGCCT TTATCCATGC TGGTTCTAGA GAAGGTGTTG TGACAAATTG CCCTTTCAGT
109321 GTGACAAATC ACCCTCAAAT GACAGTCCTG TCTGTGACAA ATTGCCCTTA ACCCTGTGAC
109381 AAATTGCCCT CAGAAGAAGC TGTTTTTTCA CAAAGTTATC CCTGCTTATT GACTCTTTTT
109441 TATTTAGTGT GACAATCTAA AAACTTGTCA CACTTCACAT GGATCTGTCA TGGCGGAAAC
109501 AGCGGTTATC AATCACAAGA AACGTGAAAA TAGCCCGCGA ATCGTCCAGT CAAACGACCT
109561 CACTGAGGCG GCATATAGTC TCTCCCGGGA TCAAAAACGT ATGCTGTATC TGTTCGTTGA
109621 CCAGATCAGA AAATCTGATG GCACCCTACA GGAACATGAC GGTATCTGCG AGATCCATGT
109681 TGCTAAATAT GCTGAAATAT TCGGATTGAC CTCTGCGGAA GCCAGTAAGG ATATACGGCA
109741 GGCATTGAAG AGTTTCGCGG GGAAGGAAGT GGTTTTTTAT CGCCCTGAAG AGGATGCCGG
109801 CGATGAAAAA GGCTATGAAT CTTTTCCTTG GTTTATCAAA CGTGCGCACA GTCCATCCAG
109861 AGGGCTTTAC AGTGTACATA TCAACCCATA TCTCATTCCC TTCTTTATCG GGTTACAGAA
109921 CCGGTTTACG CAGTTTCGGC TTAGTGAAAC AAAAGAAATC ACCAATCCGT ATGCCATGCG
109981 TTTATACGAA TCCCTGTGTC AGTATCGTAA GCCGGATGGC TCAGGCATCG TCTCTCTGAA
110041 AATCGACTGG ATCATAGAGC GTTACCAGCT GCCTCAAAGT TACCAGCGTA TGCCTGACTT
110101 CCGCCGCCGC TTCCTGCAGG TCTGTGTTAA TGAGATCAAC AGCAGAACTC CAATGCGCCT
110161 CTCATACATT GAGAAAAAGA AAGGCCGCCA GACGACTCAT ATCGTATTTT CCTTCCGCGA
110221 TATCACTTCC ATGACGACAG GATAGTCTGA GGGTTATCTG TCACAGATTT GAGGGTGGTT
110281 CGTCACATTT GTTCTGACCT ACTGAGGGTA ATTTGTCACA GTTTTGCTGT TTCCTTCAGC
110341 CTGCATGGAT TTTCTCATAC TTTTTGAACT GTAATTTTTA AGGAAGCCAA ATTTGAGGGC
110401 AGTTTGTCAC AGTTGATTTC CTTCTCTTTC CCTTCGTCAT GTGACCTGAT ATCGGGGGTT
110461 AGTTCGTCAT CATTGATGAG GGTTGATTAT CACAGTTTAT TACTCTGAAT TGGCTATCCG
110521 CGTGTGTACC TCTACCTGGA GTTTTTCCCA CGGTGGATAT TTCTTCTTGC GCTGAGCGTA
110581 AGAGCTATCT GACAGAACAG TTCTTCTTTG CTTCCTCGCC AGTTCGCTCG CTATGCTCGG
110641 TTACACGGCT GCGGCGAGCG CTAGTGATAA TAAGTGACTG AGGTATGTGC TCTTCTTATC
110701 TCCTTTTGTA GTGTTGCTCT TATTTTAAAC AACTTTGCGG TTTTTTGATG ACTTTGCGAT
110761 TTTGTTGTTG CTTTGCAGTA AATTGCAAGA TTTAATAAAA AAACGCAAAG CAATGATTAA
110821 AGGATGTTCA GAATGAAACT CATGGAAACA CTTAACCAGT GCATAAACGC TGGTCATGAA
110881 ATGACGAAGG CTATCGCCAT TGCACAGTTT AATGATGACA GCCCGGAAGC GAGGAAAATA
110941 ACCCGGCGCT GGAGAATAGG TGAAGCAGCG GATTTAGTTG GGGTTTCTTC TCAGGCTATC
111001 AGAGATGCCG AGAAAGCAGG GCGACTACCG CACCCGGATA TGGAAATTCG AGGACGGGTT
111061 GAGCAACGTG TTGGTTATAC AATTGAACAA ATTAATCATA TGCGTGATGT GTTTGGTACG
111121 CGATTGCGAC GTGCTGAAGA CGTATTTCCA CCGGTGATCG GGGTTGCTGC CCATAAAGGT
111181 GGCGTTTACA AAACCTCAGT TTCTGTTCAT CTTGCTCAGG ATCTGGCTCT GAAGGGGCTA
111241 CGTGTTTTGC TCGTGGAAGG TAACGACCCC CAGGGAACAG CCTCAATGTA TCACGGATGG
111301 GTACCAGATC TTCATATTCA TGCAGAAGAC ACTCTCCTGC CTTTCTATCT TGGGGAAAAG
111361 GACGATGTCA CTTATGCAAT AAAGCCCACT TGCTGGCCGG GGCTTGACAT TATTCCTTCC
111421 TGTCTGGCTC TGCACCGTAT TGAAACTGAG TTAATGGGCA AATTTGATGA AGGTAAACTG
111481 CCCACCGATC CACACCTGAT GCTCCGACTG GCCATTGAAA CTGTTGCTCA TGACTATGAT
111541 GTCATAGTTA TTGACAGCGC GCCTAACCTG GGTATCGGCA CGATTAATGT CGTATGTGCT
111601 GCTGATGTGC TGATTGTTCC CACGCCTGCT GAGTTGTTTG ACTACACCTC CGCACTGCAG
111661 TTTTTCGATA TGCTTCGTGA TCTGCTCAAG AACGTTGATC TTAAAGGGTT CGAGCCTGAT
111721 GTACGTATTT TGCTTACCAA ATACAGCAAT AGCAATGGCT CTCAGTCCCC GTGGATGGAG
111781 GAGCAAATTC GGGATGCCTG GGGAAGCATG GTTCTAAAAA ATGTTGTACG TGAAACGGAT
111841 GAAGTTGGTA AAGGTCAGAT CCGGATGAGA ACTGTTTTTG AACAGGCCAT TGATCAACGC
111901 TCTTCAACTG GTGCCTGGAG AAATGCTCTT TCTATTTGGG AACCTGTCTG CAATGAAATT
111961 TTCGATCGTC TGATTAAACC ACGCTGGGAG ATTAGATAAT GAAGCGTGCG CCTGTTATTC
112021 CAAAACATAC GOTCAATACT CAACCGGTTG AAGATACTTC GTTATCGACA CCAGCTGCCC
112081 CGATGGTGGA TTCGTTAATT GCGCGCGTAG GAGTAATGGC TCGCGGTAAT GCCATTACTT
112141 TGCCTGTATG TGGTCGGGAT GTGAAGTTTA CTCTTGAAGT GCTCCGGGGT GATAGTGTTG
112201 AGAAGACCTC TCGGGTATGG TCAGGTAATG AACGTGACCA GGAGCTGCTT ACTGAGGACG
112261 CACTGGATGA TCTCATCCCT TCTTTTCTAC TGACTGGTCA ACAGACACCG GCGTTCGGTC
112321 GAAGAGTATC TGGTGTCATA GAAATTGCCG ATGGGAGTCG CCGTCGTAAA GCTGCTGCAC
112381 TTACCGAAAG TGATTATCGT GTTCTGGTTG GCGAGCTGGA TGATGAGCAG ATGGCTGCAT
112441 TATCCAGATT GGGTAACGAT TATCGCCCAA CAAGTGCTTA TGAACGTGGT CAGCGTTATG
112501 CAAGCCGATT GCAGAATGAA TTTGCTGGAA ATATTTCTGC GCTGGCTGAT GCGGAAAATA
112561 TTTCACGTAA GATTATTACC CGCTGTATCA ACACCGCCAA ATTGCCTAAA TCAGTTGTTG
112621 CTCTTTTTTC TCACCCCGGT GAACTATCTG CCCGGTCAGG TGATGCACTT CAAAAAGCCT
112681 TTACAGATAA AGAGGAATTA CTTAAGCAGC AGGCATCTAA CCTTCATGAG CAGAAAAAAG
112741 CTGGGGTGAT ATTTGAAGCT GAAGAAGTTA TCACTCTTTT AACTTCTGTG CTTAAAACGT
112801 CATCTGCATC AAGAACTAGT TTAAGCTCAC GACATCAGTT TGCTCCTGGA GCGACAGTAT
112861 TGTATAAGGG CGATAAAATG GTGCTTAACC TGGACAGGTC TCGTGTTCCA ACTGAGTGTA
112921 TAGAGAAAAT TGAGGCCATT CTTAAGGAAC TTGAAAAGCC AGCACCCTGA TGCGACCTCG
112981 TTTTAGTCTA CGTTTATCTG TCTTTACTTA ATGTCCTTTG TTACAGGCCA GAAAGCATAA
113041 CTGGCCTGAA TATTCTCTCT GGGCCCACTG TTCCACTTGT ATCGTCGGTC TGATAATCAG
113101 ACTGGGACCA CGGTCCCACT CGTATCGTCG GTCTGATTAT TAGTCTGGGA CCACGGTCCC
113161 ACTCGTATCG TCGGTCTGAT TATTAGTCTG GGACCACGGT CCCACTCGTA TCGTCGGTCT
113221 GATAATCAGA CTGGGACCAC GGTCCCACTC GTATCGTCGG TCTGATTATT AGTCTGGGAC
113281 CATGGTCCCA CTCGTATCGT CGGTCTGATT ATTAGTCTGG GACCACGGTC CCACTCGTAT
113341 CGTCGGTCTG ATTATTAGTC TGGAACCACG GTCCCACTCG TATCGTCGGT CTGATTATTA
113401 GTCTGGGACC ACGGTCCCAC TCGTATCGTC GGTCTGATTA TTAGTCTGGG ACCACGATCC
113461 CACTCGTGTT GTCGGTCTGA TTATCGGTCT GGGACCACGG TCCCACTTGT ATTGTCGATC
113521 AGACTATCAG CGTGAGACTA CGATTCCATC AATGCCTGTC AAGGGCAAGT ATTGACATGT
113581 CGTCGTAACC TGTAGAACGG AGTAACCTCG GTGTGCGGTT GTATGCCTGC TGTGGATTGC
113641 TGCTGTGTCC TGCTTATCCA CAACATTTTG CGCACGGTTA TGTGGAC
(Deleter construct)
SEQ ID NO: 15
TTATTATCATGACATTAACCTATAAAAATAGGCGTATCACGAGGCCCTTTGTCACAGCTTGTCTGTAAGCGGATGCCGG
GAGCAGACAAGCCCGTCAGGGCGCGTCAGCGGGTGTTGGCGGGTGTCGGGGCTGGCTTAACTATGCGGCATCAGAGCAG
ATTGTACTGAGAGTGCACCATACCACAGCTTTTCAATTCAATTCATCATTTTTTTTTTATTCTTTTTTTTGATTTCGGT
TTCTTTGAAATTTTTTTGATTCGGTAATCTCCGAACAGAAGGAAGAACGAAGGAAGGAGCACAGACTTAGATTGGTATA
TATACGCATATGTAGTGTTGAAGAAACATGAAATTGCCCAGTATTCTTAACCCAACTGCACAGAACAAAAACCTGCAGG
AAACGAAGATAAATCATGTCGAAAGCTACATATAAGGAACGTGCTGCTACTCATCCTAGTCCTGTTGCTGCCAAGCTAT
TTAATATCATGCACGAAAAGCAAACAAACTTGTGTGCTTCATTGGATGTTCGTACCACCAAGGAATTACTGGAGTTAGT
TGAAGCATTAGGTCCCAAAATTTGTTTACTAAAAACACATGTGGATATCTTGACTGATTTTTCCATGGAGGGCACAGTT
AAGCCGCTAAAGGCATTATCCGCCAAGTACAATTTTTTACTCTTCGAAGACAGAAAATTTGCTGACATTGGTAATACAG
TCAAATTGCAGTACTCTGCGGGTGTATACAGAATAGCAGAATGGGCAGACATTACGAATGCACACGGTGTGGTGGGCCC
AGGTATTGTTAGCGGTTTGAAGCAGGCGGCAGAAGAAGTAACAAAGGAACCTAGAGGCCTTTTGATGTTAGCAGAATTG
TCATGCAAGGGCTCCCTATCTACTGGAGAATATACTAAGGGTACTGTTGACATTGCGAAGAGCGACAAAGATTTTGTTA
TCGGCTTTATTGCTCAAAGAGACATGGGTGGAAGAGATGAAGGTTACGATTGGTTGATTATGACACCCGGTGTGGGTTT
AGATGACAAGGGAGATGCATTGGGTCAACAGTATAGAACCGTGGATGATGTGGTGTCTACAGGATCTGACATTATTATT
GTTGGAAGAGGACTATTTGCAAAGGGAAGGGATGCTAAGGTAGAGGGTGAACGTTACAGAAAAGCAGGCTGGGAAGCAT
ATTTGAGAAGATGCGGCCAGCAAAACTAAAAAACTGTATTATAAGTAAATGCATGTATACTAAACTCACAAATTAGAGC
TTCAATTTAATTATATCAGTTATTACCCTATGCGGTGTGAAATACCGCACAGATGCGTAAGGAGAAAATACCGCATCAG
GAAATTGTAAACGTTAATATTTTGTTAAAATTCGCGTTAAATTTTTGTTAAATCAGCTCATTTTTTAACCAATAGGCCG
AAATCGGCAAAATCCCTTATAAATCAAAAGAATAGACCGAGATAGGGTTGAGTGTTGTTCCAGTTTGGAACAAGAGTCC
ACTATTAAAGAACGTGGACTCCAACGTCAAAGGGCGAAAAACCGTCTATCAGGGCGATGGCCCACTACGTGAACCATCA
CCCTAATCAAGTTTTTTGGGGTCGAGGTGCCGTAAAGCACTAAATCGGAACCCTAAAGGGAGCCCCCGATTTAGAGCTT
GACGGGGAAAGCCGGCGAACGTGGCGAGAAAGGAAGGGAAGAAAGCGAAAGGAGCGGGCGCTAGGGCGCTGGCAAGTGT
AGCGGTCACGCTGCGCGTAACCACCACACCCGCCGCGCTTAATGCGCCGCTACAGGGCGCGTCGCGCCATTCGCCATTC
AGGCTGCGCAACTGTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTGAGCGCGCGTAATA
CGACTCACTATAGGGCGAATTGGGTACCGCGAGACCTTCCGGGTCTCAGAAGTTCCTATACTTCAAAAGGAATAGGAAC
TTCGGATCCGGGTAGGGGAGGCGCTTTTCCCAAGGCAGTCTGGAGCATGCGCTTTAGCAGCCCCGCTGGGCACTTGGCG
CTACACAAGTGGCCTCTGGCCTCGCACACATTCCACATCCACCGGTAGGCGCCAACCGGCTCCGTTCTTTGGTGGCCCC
TTCGCGCCACCTTCTACTCCTCCCCTAGTCAGGAAGTTCCCCCCCGCCCCGCAGCTCGCGTCGTGCAGGACGTGACAAA
TGGAAGTAGCACGTCTCACTAGTCTCGTGCAGATGGACAGCACCGCTGAGCAATGGAAGCGGGTAGGCCTTTGGGGCAG
CGGCCAATAGCAGCTTTGCTCCTTCGCTTTCTGGGCTCAGAGGCTGGGAAGGGGTGGGTCCGGGGGCGGGCTCAGGGGC
GGGCTCAGGGGCGGGGCGGGCGCCCGAAGGTCCTCCGGAGGCCCGGCATTCTGCACGCTTCAAAAGCGCACGTCTGCCG
CGCTGTTCTCCTCTTCCTCATCTCCGGGCCTTTCGACCGATCATCAAGCTTGATCCTCATGAAAAAGCCTGAACTCACC
GCGACGTCTGTCGAGAAGTTTCTGATCGAAAAGTTCGACAGCGTCTCCGACCTGATGCAGCTCTCGGAGGGCGAAGAAT
CTCGTGCTTTCAGCTTCGATGTAGGAGGGCGTGGATATGTCCTGCGGGTAAATAGCTGCGCCGATGGTTTCTACAAAGA
TCGTTATGTTTATCGGCACTTTGCATCGGCCGCGCTCCCGATTCCGGAAGTGCTTGACATTGGGGAATTCAGCGAGAGC
CTGACCTATTGCATCTCCCGCCGTGCACAGGGTGTCACGTTGCAAGACCTGCCTGAAACCGAACTGCCCGCTGTTCTGC
AGCCGGTCGCGGAGGCCATGGATGCGATCGCTGCGGCCGATCTTAGCCAGACGAGCGGGTTCGGCCCATTCGGACCGCA
AGGAATCGGTCAATACACTACATGGCGTGATTTCATATGCGCGATTGCTGATCCCCATGTGTATCACTGGCAAACTGTG
ATGGACGACACCGTCAGTGCGTCCGTCGCGCAGGCTCTCGATGAGCTGATGCTTTGGGCCGAGGACTGCCCCGAAGTCC
GGCACCTCGTGCACGCGGATTTCGGCTCCAACAATGTCCTGACGGACAATGGCCGCATAACAGCGGTCATTGACTGGAG
CGAGGCGATGTTCGGGGATTCCCAATACGAGGTCGCCAACATCTTCTTCTGGAGGCCGTGGTTGGCTTGTATGGAGCAG
CAGACGCGCTACTTCGAGCGGAGGCATCCGGAGCTTGCAGGATCGCCGCGGCTCCGGGCGTATATGCTCCGCATTGGTC
TTGACCAACTCTATCAGAGCTTGGTTGACGGCAATTTCGATGATGCAGCTTGGGCGCAGGGTCGATGCGACGCAATCGT
CCGATCCGGAGCCGGGACTGTCGGGCGTACACAAATCGCCCGCAGAAGCGCGGCCGTCTGGACCGATGGCTGTGTAGAA
GTCGCGTCTGCGTTCGACCAGGCTGCGCGTTCTCGCGGCCATAGCAACCGACGTACGGCGTTGCGCCCTCGCCGGCAGC
AAGAAGCCACGGAAGTCCGCCCGGAGCAGAAAATGCCCACGCTACTGCGGGTTTATATAGACGGTCCCCACGGGATGGG
GAAAACCACCACCACGCAACTGCTGGTGGCCCTGGGTTCGCGCGACGATATCGTCTACGTACCCGAGCCGATGACTTAC
TGGCGGGTGCTGGGGGCTTCCGAGACAATCGCGAACATCTACACCACACAACACCGCCTCGACCAGGGTGAGATATCGG
CCGGGGACGCGGCGGTGGTAATGACAAGCGCCCAGATAACAATGGGCATGCCTTATGCCGTGACCGACGCCGTTCTGGC
TCCTCATATCGGGGGGGAGGCTGGGAGCTCACATGCCCCGCCCCCGGCCCTCACCCTCATCTTCGACCGCCATCCCATC
GCCGCCCTCCTGTGCTACCCGGCCGCGCGGTACCTTATGGGCAGCATGACCCCCCAGGCCGTGCTGGCGTTCGTGGCCC
TCATCCCGCCGACCTTGCCCGGCACCAACATCGTGCTTGGGGCCCTTCCGGAGGACAGACACATCGACCGCCTGGCCAA
ACGCCAGCGCCCCGGCGAGCGGCTGGACCTGGCTATGCTGGCTGCGATTCGCCGCGTTTACGGGCTACTTGCCAATACG
GTGCGGTATCTGCAGTGCGGCGGGTCGTGGCGGGAGGACTGGGGACAGCTTTCGGGGACGGCCGTGCCGCCCCAGGGTG
CCGAGCCCCAGAGCAACGCGGGCCCACGACCCCATATCGGGGACACGTTATTTACCCTGTTTCGGGCCCCCGAGTTGCT
GGCCCCCAACGGCGACCTGTATAACGTGTTTGCCTGGGCCTTGGACGTCTTGGCCAAACGCCTCCGTTCCATGCACGTC
TTTATCCTGGATTACGACCAATCGCCCGCCGGCTGCCGGGACGCCCTGCTGCAACTTACCTCCGGGATGGTCCAGACCC
ACGTCACCACCCCCGGCTCCATACCGACGATATGCGACCTGGCGCGCACGTTTGCCCGGGAGATGGGGGAGGCTAACTG
AGTCGACTAAGATCATAATCAGCCATACCACATTTGTAGAGGTTTTACTTGCTTTAAAAAACCTCCCACACCTCCCCCT
GAACCTGAAACATAAAATGAATGCAATTGTTGTTGTTAACTTGTTTATTGCAGCTTATAATGGTTACAAATAAAGCAAT
AGCATCACAAATTTCACAAATAAAGCATTTTTTTCACTGCATTCTAGTTGTGGTTTGTCCAAACTCATCAATGTATCTT
ATCATGTCTGGGAAGTTCCTATACTTCAAAAGGAATAGGAACTTCTTGTAGAGACCAACCGGGTCTCAGCTAGCTCCCT
TTAGTGAGGGTTAATTGCGCGCTTGGCGTAATCATGGTCATAGCTGTTTCCTGTGTGAAATTGTTATCCGCTCACAATT
CCACACAACATAGGAGCCGGAAGCATAAAGTGTAAAGCCTGGGGTGCCTAATGAGTGAGGTAACTCACATTAATTGCGT
TGCGCTCACTGCCCGCTTTCCAGTCGGGAAACCTGTCGTGCCAGCTGCATTAATGAATCGGCCAACGCGCGGGGAGAGG
CGGTTTGCGTATTGGGCGCTCTTCCGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCAGGCGTTTCCCCCTGGAAGCTCCCTC
GTGCGCTCTCCTGTTCCGACCCTGCCGCTTACCGGATACCTGTCCGCCTTTCTCCCTTCGGGAAGCGTGGCGCTTTCTC
ATAGCTCACGCTGTAGGTATCTCAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGGTGGCCTAACTACGGCT
ACACTAGAAGGACAGTATTTGGTATCTGCGCTCTGCTGAAGCCAGTTACCTTCGGAAAAAGAGTTGGTAGCTCTTGATC
CGGCAAACAAACCACCGCTGGTAGCGGTGGTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAACTCACGTTAAGGGATTTTGGTCATGAGAT
TATCAAAAAGGATCTTCACCTAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCTATTTCGTTCATCCATAGTTGCC
TGACTCCCCGTCGTGTAGATAACTACGATACGGGAGGGCTTACCATCTGGCCCCAGTGCTGCAATGATACCGCGAGATC
CACGCTCACCGGCTCCAGATTTATCAGCAATAAACCAGCCAGCCGGAAGGGCCGAGCGCAGAAGTGGTCCTGCAACTTT
ATCCGCCTCCATCCAGTCTATTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAGCTCCGGTTCCCAACGATCAA
GGCGAGTTACATGATCCCCCATGTTGTGCAAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCCGGCGTCAATAC
GGGATAATACCGCGCCACATAGCAGAACTTTAAAAGTGCTCATCATTGGAAAACGTTCTTCGGGGCGAAAACTCTCAAG
GATCTTACCGCTGTTGAGATCCAGTTCGATGTAACCCACTCGTGCACCCAACTGATCTTCAGCATCTTTTACTTTCACC
AGCGTTTCTGGGTGAGCAAAAACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGGGTCCTTTTCATCACGTGCTATA
AAAATAATTATAATTTAAATTTTTTAATATAAATATATAAATTAAAAATAGAAAGTAAAAAAAGAAATTAAAGAAAAAA
TAGTTTTTGTTTTCCGAAGATGTAAAAGACTCTAGGGGGATCGCCAACAAATACTACCTTTTATCTTGCTCTTCCTGCT
CTCAGGTATTAATGCCGAATTGTTTCATCTTGTCTGTGTAGAAGACCACACACGAAAATCCTGTGATTTTACATTTTAC
TTATCGTTAATCGAATGTATATCTATTTAATCTGCTTTTCTTGTCTAATAAATATATATGTAAAGTACGCTTTTTGTTG
AAATTTTTTAAACCTTTGTTTATTTTTTTTTCTTCATTCCGTAACTCTTCTACCTTCTTTATTTACTTTCTAAAATCCA
AATACAAAACATAAAAATAAATAAACACAGAGTAAATTCCCAAATTATTCCATCATTAAAAGATACGAGGCGCGTGTAA
GTTACAGGCAAGCGATCCGTCCTAAGAAACCA
(REWRITE minimal payload pDT3.fa)
SEQ ID NO: 16
CCCCGAAAAGTGCCACCTGGGTCCTTTTCATCACGTGCTATAAAAATAATTATAATTTAAATTTTTTAATATAAATATA
TAAATTAAAAATAGAAAGTAAAAAAAGAAATTAAAGAAAAAATAGTTTTTGTTTTCCGAAGATGTAAAAGACTCTAGGG
GGATCGCCAACAAATACTACCTTTTATCTTGCTCTTCCTGCTCTCAGGTATTAATGCCGAATTGTTTCATCTTGTCTGT
GTAGAAGACCACACACGAAAATCCTGTGATTTTACATTTTACTTATCGTTAATCGAATGTATATCTATTTAATCTGCTT
TTCTTGTCTAATAAATATATATGTAAAGTACGCTTTTTGTTGAAATTTTTTAAACCTTTGTTTATTTTTTTTTCTTCAT
TCCGTAACTCTTCTACCTTCTTTATTTACTTTCTAAAATCCAAATACAAAACATAAAAATAAATAAACACAGAGTAAAT
TCCCAAATTATTCCATCATTAAAAGATACGAGGCGCGTGTAAGTTACAGGCAAGCGATCCGTCCTAAGAAACCATTATT
ATCATGACATTAACCTATAAAAATAGGCGTATCACGAGGCCCTTTCGTCTCGCGCGTTTCGGTGATGACGGTGAAAACC
TCTGACACATGCAGCTCCCGGAGACGGTCACAGCTTGTCTGTAAGCGGATGCCGGGAGCAGACAAGCCCGTCAGGGCGC
GTCAGCGCGTGTTGGCGGGTGTCGGGGCTGGCTTAACTATGCGGCATCAGAGCAGATTGTACTGAGAGTGCACCATAAA
TTCCCGTTTTAAGAGCTTGGTGAGCGCTAGGAGTCACTGCCAGGTATCGTTTGAACACGGCATTAGTCAGGGAAGTCAT
AACACAGTCCTTTCCCGCAATTTTCTTTTTCTATTACTCTTGGCCTCCTCTAGTACACTCTATATTTTTTTATGCCTCG
GTAATGATTTTCATTTTTTTTTTTCCCCTAGCGGATGACTCTTTTTTTTTCTTAGCGATTGGCATTATCACATAATGAA
TTATACATTATATAAAGTAATGTGATTTCTTCGAAGAATATACTAAAAAATGAGCAGGCAAGATAAACGAAGGCAAAGA
TGACAGAGCAGAAAGCCCTAGTAAAGCGTATTACAAATGAAACCAAGATTCAGATTGCGATCTCTTTAAAGGGTGGTCC
CCTAGCGATAGAGCACTCGATCTTCCCAGAAAAAGAGGCAGAAGCAGTAGCAGAACAGGCCACACAATCGCAAGTGATT
AACGTCCACACAGGTATAGGGTTTCTGGACCATATGATACATGCTCTGGCCAAGCATTCCGGCTGGTCGCTAATCGTTG
AGTGCATTGGTGACTTACACATAGACGACCATCACACCACTGAAGACTGCGGGATTGCTCTCGGTCAAGCTTTTAAAGA
GGCCCTACTGGCGCGTGGAGTAAAAAGGTTTGGATCAGGATTTGCGCCTTTGGATGAGGCACTTTCCAGAGCGGTGGTA
GATCTTTCGAACAGGCCGTACGCAGTTGTCGAACTTGGTTTGCAAAGGGAGAAAGTAGGAGATCTCTCTTGCGAGATGA
TCCCGCATTTTCTTGAAAGCTTTGCAGAGGCTAGCAGAATTACCCTCCACGTTGATTGTCTGCGAGGCAAGAATGATCA
TCACCGTAGTGAGAGTGCGTTCAAGGCTCTTGCGGTTGCCATAAGAGAAGCCACCTCGCCCAATGGTACCAACGATGTT
CCCTCCACCAAAGGTGTTCTTATGTAGTGACACCGATTATTTAAAGCTGCAGCATACGATATATATACATGTGTATATA
TGTATACCTATGAATGTCAGTAAGTATGTATACGAACAGTATGATACTGAAGATGACAAGGTAATGCATCATTCTATAC
GTGTCATTCTGAACGAGGCGCGCTTTCCTTTTTTCTTTTTGCTTTTTCTTTTTTTTTCTCTTGAACTCGACGGATCTAT
GCGGTGTGAAATACCGCACAGATGCGTCCTAGGGCGGCCGCATAACTTCGTATAGCATACATTATACGAAGTTATCCGG
AACCGAGTACAAGCCCACGGTGCGCCTCGCCACCCGCGACGACGTCCCCAGGGCCGTCCGCACCCTCGCCGCCGCGTTC
GCCGACTACCCCGCCACGCGCCACACCGTCGATCCGGACCGCCACATCGAGCGCGTGACCGAGCTGCAAGAACTCTTCC
TCACGCGCGTCGGGCTCGACATCGGCAAGGTGTGGGTCGCGGACGACGGAGCCGCTGTGGCGGTCTGGACCACGCCGGA
GAGCGTCGAAGCGGGGGCGGTGTTCGCCGAGATCGGCCCGCGCATGGCCGAGTTGAGCGGTTCCCGGCTGGCCGCGCAG
CAACAGATGGAGGGCCTCCTGGCTCCGCACCGGCCCAAGGAGCCCGCGTGGTTCCTGGCCACCGTCGGAGTCTCGCCCG
ACCACCAGGGCAAGGGTCTGGGCAGCGCCGTCGTGCTCCCCGGAGTGGAGGCGGCCGAGCGCGCCGGGGTGCCCGCCTT
CCTGGAAACCTCCGCGCCCCGCAACCTCCCCTTCTACGAGCGGCTCGGCTTCACCGTCACCGCCGACGTCGAGGTGCCC
GAAGGACCGCGCACCTGGTGCATGACCCGCAAGCCCGGTGCCGCATGCCCCACGCTACTGCGGGTTTATATAGACGGTC
CCCACGGGATGGGGAAAACCACCACCACGCAACTGCTGGTGGCCCTGGGTTCGCGCGACGATATCGTCTATGTACCCGA
GCCGATGACTTACTGGCGGGTGCTGGGGGCTTCCGAGACAATCGCGAACATCTACACCACACAACACCGCCTCGACCAG
GGTGAGATATCGGCCGGGGACGCGGCGGTGGTAATGACAAGCGCCCAGATAACAATGGGTATGCCTTATGCCGTGACCG
ACGCCGTTCTGGCTCCTCATATCGGGGGGGAGGCTGGTAGCTCACATGCCCCGCCCCCGGCCCTCACCCTCATCTTCGA
CCGCCATCCCATCGCCGCCCTCCTGTGCTACCCGGCCGCGCGGTACCTTATGGGCAGCATGACCCCCCAGGCCGTGCTG
GCGTTCGTGGCCCTCATCCCGCCGACCTTGCCCGGCACCAACATCGTGCTTGGGGCCCTGCCGGAGGACAGACACATCG
ACCGCCTGGCCAAACGCCAGCGCCCCGGCGAGCGGCTGGACCTGGCTATGCTGGCTGCGATTCGCCGCGTTTACGGGCT
ACTTGCCAATACGGTGCGGTATCTGCAGTGCGGCGGGTCGTGGCGGGAGGACTGGGGACAGCTTTCGGGGACGGCCGTG
CCGCCCCAGGGTGCCGAGCCCCAGAGCAACGCGGGCCCACGACCCCATATCGGGGACACGTTATTTACCCTGTTTCGGG
CCCCCGAGTTGCTGGCCCCCAACGGCGACCTGTATAACGTGTTTGCCTGGGCCTTGGACGTCTTGGCCAAACGCCTCCG
TTCCATGCACGTCTTTATCCTGGATTACGACCAATCGCCCGCCGGCTGCCGGGACGCCCTGCTGCAACTTACCTCCGGG
ATGGTCCAGACCCACGTCACCACCCCCGGCTCCATACCGACGATATGCGACCTGGCGCGCACGTTTGCCCGCGAGATGG
GGGAGGCTAACGAGCTCGGCAGTGGAGAGGGCAGAGGAAGTCTGCTAACATGCGGTGACGTCGAGGAGAATCCTGGCCC
AAGCCAGTTCGACATCCTGTGCAAGACCCCCCCCAAGGTGCTGGTGCGGCAGTTCGTGGAGAGATTCGAGAGGCCCAGC
GGCGAGAAGATCGCCAGCTGTGCCGCCGAGCTGACCTACCTGTGCTGGATGATCACCCACAACGGCACCGCCATCAAGA
GGGCCACCTTCATGAGCTACAACACCATCATCAGCAACAGCCTGAGCTTCGACATCGTGAACAAGAGCCTGCAGTTCAA
GTACAAGACCCAGAAGGCCACCATCCTGGAGGCCAGCCTGAAGAAGCTGATCCCCGCCTGGGAGTTCACCATCATCCCT
TACAACGGCCAGAAGCACCAGAGCGACATCACCGACATCGTGTCCAGCCTGCAGCTGCAGTTCGAGAGCAGCGAGGAGG
CCGACAAGGGCAACAGCCACAGCAAGAAGATGCTGAAGGCCCTGCTGTCCGAGGGCGAGAGCATCTGGGAGATCACCGA
GAAGATCCTGAACAGCTTCGAGTACACCAGCAGGTTCACCAAGACCAAGACCCTGTACCAGTTCCTGTTCCTGGCCACA
TTCATCAACTGCGGCAGGTTCAGCGACATCAAGAACGTGGACCCCAAGAGCTTCAAGCTGGTGCAGAACAAGTACCTGG
GCGTGATCATTCAGTGCCTGGTGACAGAAACCAAGACAAGCGTGTCCAGGCACATCTACTTTTTCAGCGCCAGAGGCAG
GATCGACCCCCTGGTGTACCTGGACGAGTTTCTGAGGAACAGCGAGCCCGTGCTGAAGAGAGTGAACAGGACCGGCAAC
AGCAGCAGCAACAAGCAGGAGTACCAGCTGCTGAAGGACAACCTGGTGCGCAGCTACAACAAGGCCCTGAAGAAGAACG
CCCCCTACCCCATCTTCGCTATCAAGAACGGCCCTAAGAGCCACATCGGCAGGCACCTGATGACCAGCTTTCTGAGCAT
GAAGGGCCTGACCGAGCTGACAAACGTGGTGGGCAACTGGAGCGACAAGAGGGCCTCCGCCGTGGCCAGGACCACCTAC
ACCCACCAGATCACCGCCATCCCCGACCACTACTTCGCCCTGGTGTCCAGGTACTACGCCTACGACCCCATCAGCAAGG
AGATGATCGCCCTGAAGGACGAAACCAACCCCATCGAGGAGTGGCAGCACATCGAGCAGCTGAAGGGCAGCGCCGAGGG
CAGCATCAGATACCCCGCCTGGAACGGCATCATCAGCCAGGAGGTGCTGGACTACCTGAGCAGCTACATCAACAGGCGG
ATCACCGGTTGCGTACGCGGATCCTCTGCTGGAGACATGAGAGCTGCCAACCTTTGGCCAAGCCCGCTCATGATCAAAC
GCTCTAAGAAGAACAGCCTGGCCTTGTCCCTGACGGCCGACCAGATGGTCAGTGCCTTGTTGGATGCTGAGCCCCCCAT
ACTCTATTCCGAGTATGATCCTACCAGACCCTTCAGTGAAGCTTCGATGATGGGCTTACTGACCAACCTGGCAGACAGG
GAGCTGGTTCACATGATCAACTGGGCGAAGAGGGTGCCAGGCTTTGTGGATTTGACCCTCCATGATCAGGTCCACCTTC
TAGAATGTGCCTGGCTAGAGATCCTGATGATTGGTCTCGTCTGGCGCTCCATGGAGCACCCAGTGAAGCTACTGTTTGC
TCCTAACTTGCTCTTGGACAGGAACCAGGGAAAATGTGTAGAGGGCATGGTGGAGATCTTCGACATGCTGCTGGCTACA
TCATCTCGGTTCCGCATGATGAATCTGCAGGGAGAGGAGTTTGTGTGCCTCAAATCTATTATTTTGCTTAATTCTGGAG
TGTACACATTTCTGTCCAGCACCCTGAAGTCTCTGGAAGAGAAGGACCATATCCACCGAGTCCTGGACAAGATCACAGA
CACTTTGATCCACCTGATGGCCAAGGCAGGCCTGACCCTGCAGCAGCAGCACCAGCGGCTGGCCCAGCTCCTCCTCATC
CTCTCCCACATCAGGCACATGAGTAACAAAGGCATGGAGCATCTGTACAGCATGAAGTGCAAGAACGTGGTGCCCCTCT
ATGACCTGCTGCTGGAGGCGGCGGACGCCCACCGCCTACATGCGCCCACTAGCCGTGGAGGGGCATCCGTGGAGGAGAC
GGACCAAAGCCACTTGGCCACTGCGGGCTCTACTTCATCGCATTCCTTGCAAAAGTATTACATCACGGGGGAGGCAGAG
GGTTTCCCTGCCACAGCTGGCTCCGGCGAGGGCAGGGGAAGTCTTCTAACATGCGGGGACGTGGAGGAAAATCCCGGCC
CAGTGAGCAAGGGTGAGGAGGATAACATGGCCTCTCTCCCAGCGACACATGAGTTACACATCTTTGGCTCCATCAACGG
TGTGGACTTTGACATGGTGGGTCAGGGCACCGGCAATCCAAATGATGGTTATGAGGAGTTAAACCTGAAGTCCACCAAG
GGCGACCTCCAGTTCTCCCCCTGGATTCTGGTCCCTCATATCGGGTATGGCTTCCATCAGTACCTGCCCTACCCTGACG
GGATGTCGCCTTTCCAGGCCGCCATGGTAGATGGCTCTGGATACCAAGTCCATCGCACAATGCAGTTTGAAGATGGTGC
CTCCCTTACTGTGAACTACCGCTACACCTACGAGGGAAGCCACATCAAAGGAGAGGCCCAGGTGAAGGGGACTGGTTTC
CCTGCTGACGGTCCTGTGATGACCAACTCGCTGACCGCTGCGGACTGGTGCAGGTCGAAGAAGACTTACCCCAACGACA
AAACCATCATCAGTACCTTTAAGTGGAGTTACACCACTGGAAATGGCAAGCGCTACCGGAGCACTGCGCGGACCACCTA
CACCTTTGCCAAGCCAATGGCGGCTAACTATCTGAAGAACCAGCCGATGTACGTGTTCCGTAAGACGGAACTCAAGCAC
TCCAAGACCGAGCTGAACTTCAAGGAGTGGCAAAAGGCGTTTACCGATGTGATGGGCATGGACGAGCTGTACAAGTGAA
TTCGATCATAATCAGCCATACCACATTTGTAGAGGTTTTACTTGCTTTAAAAAACCTCCCACACCTCCCCCTGAACCTG
AAACATAAAATGAATGCAATTGTTGTTGTTAACTTGTTTATTGCAGCTTATAATGGTTACAAATAAAGCAATAGCATCA
CAAATTTCACAAATAAAGCATTTTTTTCACTGCATTCTAGTTGTGGTTTGTCCAAACTCATCAATGTATCTTATCATGT
CTGGCTCGAGGGAAGTTCCTATACTCTAGAAAGAATAGGAACTTCGGCGGACGCGTATAGATCAGGCGCGCCACAACTT
CGTATAGAAAGGTATATACGAAGTTGTGCGGCCGCGTCGACCAATTCTCATGTTTGACAGCTTATCATCGAATTTCTGC
CATTCATCCGCTTATTATCACTTATTCAGGCGTAGCAACCAGGCGTTTAAGGGCACCAATAACTGCCTTAAAAAAATTA
GAAAAACTCATCGAGCATCAAATGAAACTGCAATTTATTCATATCAGGATTATCAATACCATATTTTTGAAAAAGCCGT
TTCTGTAATGAAGGAGAAAACTCACCGAGGCAGTTCCATAGGATGGCAAGATCCTGGTATCGGTCTGCGATTCCGACTC
GTCCAACATCAATACAACCTATTAATTTCCCCTCGTCAAAAATAAGGTTATCAAGTGAGAAATCACCATGAGTGACGAC
TGAATCCGGTGAGAATGGCAAAAGCTTATGCATTTCTTTCCAGACTTGTTCAACAGGCCAGCCATTACGCTCGTCATCA
AAATCACTCGCATCAACCAAACCGTTATTCATTCGTGATTGCGCCTGAGCGAGACGAAATACGCGATCGCTGTTAAAAG
GACAATTACAAACAGGAATCGAATGCAACCGGCGCAGGAACACTGCCAGCGCATCAACAATATTTTCACCTGAATCAGG
ATATTCTTCTAATACCTGGAATGCTGTTTTCCCTGGGATCGCAGTGGTGAGTAACCATGCATCATCAGGAGTACGGATA
AAATGCTTGATGGTCGGAAGAGGCATAAATTCCGTCAGCCAGTTTAGTCTGACCATCTCATCTGTAACATCATTGGCAA
CGCTACCTTTGCCATGTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGCGGCCTCGAGCAAGAC
GTTTCCCGTTGAATATGGCTCATTTTAGCTTCCTTAGCTCCTGAAAATCTCGATAACTCAAAAAATACGCCCGGTAGTG
ATCTTATTTCATTATGGTGAAAGTTGGAACCTCTTACGTGCCGATCAACGTCTCATTTTCGCCAAAAGTTGGCCCAGGG
CTTCCCGGTATCAACAGGGACACCAGGATTTATTTATTCTGCGAAGTGATCTTCCGTCACAGGTATTTATTCGCGATAA
GCTCATGGAGCGGCGTAACCGTCGCACAGGAAGGACAGAGAAAGCGCGGATCTGGGAAGTGACGGACAGAACGGTCAGG
ACCTGGATTGGGGAGGCGGTTGCCGCCGCTGCTGCTGACGGTGTGACGTTCTCTGTTCCGGTCACACCACATACGTTCC
GCCATTCCTATGCGATGCACATGCTGTATGCCGGTATACCGCTGAAAGTTCTGCAAAGCCTGATGGGACATAAGTCCAT
CAGTTCAACGGAAGTCTACACGAAGGTTTTTGCGCTGGATGTGGCTGCCCGGCACCGGGTGCAGTTTGCGATGCCGGAG
TCTGATGCGGTTGCGATGCTGAAACAATTATCCTGAGAATAAATGCCTTGGCCTTTATATGGAAATGTGGAACTGAGTG
GATATGCTGTTTTTGTCTGTTAAACAGAGAAGCTGGCTGTTATCCACTGAGAAGCGAACGAAACAGTCGGGAAAATCTC
CCATTATCGTAGAGATCCGCATTATTAATCTCAGGAGCCTGTGTAGCGTTTATAGGAAGTAGTGTTCTGTCATGATGCC
TGCAAGCGGTAACGAAAACGATTTGAATATGCCTTCAGGAACAATAGAAATCTTCGTGCGGTGTTACGTTGAAGTGGAG
CGGATTATGTCAGCAATGGACAGAACAACCTAATGAACACAGAACCATGATGTGGTCTGTCCTTTTACAGCCAGTAGTG
CTCGCCGCAGTCGAGCGACAGGGCGAAGCCCTCGAGCTGGTTGCCCTCGCCGCTGGGCTGGCGGCCGTCTATGGCCCTG
CAAACGCGCCAGAAACGCCGTCGAAGCCGTGTGCGAGACACCGCGGCCGGCCGCCGGCGTTGTGGATACCTCGCGGAAA
ACTTGGCCCTCACTGACAGATGAGGGGCGGACGTTGACACTTGAGGGGCCGACTCACCCGGCGCGGCGTTGACAGATGA
GGGGCAGGCTCGATTTCGGCCGGCGACGTGGAGCTGGCCAGCCTCGCAAATCGGCGAAAACGCCTGATTTTACGCGAGT
TTCCCACAGATGATGTGGACAAGCCTGGGGATAAGTGCCCTGCGGTATTGACACTTGAGGGGCGCGACTACTGACAGAT
GAGGGGCGCGATCCTTGACACTTGAGGGGCAGAGTGCTGACAGATGAGGGGCGCACCTATTGACATTTGAGGGGCTGTC
CACAGGCAGAAAATCCAGCATTTGCAAGGGTTTCCGCCCGTTTTTCGGCCACCGCTAACCTGTCTTTTAACCTGCTTTT
AAACCAATATTTATAAACCTTGTTTTTAACCAGGGCTGCGCCCTGTGCGCGTGACCGCGCACGCCGAAGGGGGGTGCCC
CCCCTTCTCGAACCCTCCCGGTCGAGTGAGCGAGGAAGCACCAGGGAACAGCACTTATATATTCTGCTTACACACGATG
CCTGAAAAAACTTCCCTTGGGGTTATCCACTTATCCACGGGGATATTTTTATAATTATTTTTTTTATAGTTTTTAGATC
TTCTTTTTTAGAGCGCCTTGTAGGCCTTTATCCATGCTGGTTCTAGAGAAGGTGTTGTGACAAATTGCCCTTTCAGTGT
GACAAATCACCCTCAAATGACAGTCCTGTCTGTGACAAATTGCCCTTAACCCTGTGACAAATTGCCCTCAGAAGAAGCT
GTTTTTTCACAAAGTTATCCCTGCTTATTGACTCTTTTTTATTTAGTGTGACAATCTAAAAACTTGTCACACTTCACAT
GGATCTGTCATGGCGGAAACAGCGGTTATCAATCACAAGAAACGTAAAAATAGCCCGCGAATCGTCCAGTCAAACGACC
TCACTGAGGCGGCATATAGTCTCTCCCGGGATCAAAAACGTATGCTGTATCTGTTCGTTGACCAGATCAGAAAATCTGA
TGGCACCCTACAGGAACATGACGGTATCTGCGAGATCCATGTTGCTAAATATGCTGAAATATTCGGATTGACCTCTGCG
GAAGCCAGTAAGGATATACGGCAGGCATTGAAGAGTTTCGCGGGGAAGGAAGTGGTTTTTTATCGCCCTGAAGAGGATG
CCGGCGATGAAAAAGGCTATGAATCTTTTCCTTGGTTTATCAAACGTGCGCACAGTCCATCCAGAGGGCTTTACAGTGT
ACATATCAACCCATATCTCATTCCCTTCTTTATCGGGTTACAGAACCGGTTTACGCAGTTTCGGCTTAGTGAAACAAAA
GAAATCACCAATCCGTATGCCATGCGTTTATACGAATCCCTGTGTCAGTATCGTAAGCCGGATGGCTCAGGCATCGTCT
CTCTGAAAATCGACTGGATCATAGAGCGTTACCAGCTGCCTCAAAGTTACCAGCGTATGCCTGACTTCCGCCGCCGCTT
CCTGCAGGTCTGTGTTAATGAGATCAACAGCAGAACTCCAATGCGCCTCTCATACATTGAGAAAAAGAAAGGCCGCCAG
ACGACTCATATCGTATTTTCCTTCCGCGATATCACTTCCATGACGACAGGATAGTCTGAGGGTTATCTGTCACAGATTT
GAGGGTGGTTCGTCACATTTGTTCTGACCTACTGAGGGTAATTTGTCACAGTTTTGCTGTTTCCTTCAGCCTGCATGGA
TTTTCTCATACTTTTTGAACTGTAATTTTTAAGGAAGCCAAATTTGAGGGCAGTTTGTCACAGTTGATTTCCTTCTCTT
TCCCTTCGTCATGTGACCTGATATCGGGGGTTAGTTCGTCATCATTGATGAGGGTTGATTATCACAGTTTATTACTCTG
AATTGGCTATCCGCGTGTGTACCTCTACCTGGAGTTTTTCCCACGGTGGATATTTCTTCTTGCGCTGAGCGTAAGAGCT
ATCTGACAGAACAGTTCTTCTTTGCTTCCTCGCCAGTTCGCTCGCTATGCTCGGTTACACGGCTGCGGCGAGCGCTAGT
GATAATAAGTGACTGAGGTATGTGCTCTTCTTATCTCCTTTTGTAGTGTTGCTCTTATTTTAAACAACTTTGCGGTTTT
TTGATGACTTTGCGATTTTGTTGTTGCTTTGCAGTAAATTGCAAGATTTAATAAAAAAACGCAAAGCAATGATTAAAGG
ATGTTCAGAATGAAACTCATGGAAACACTTAACCAGTGCATAAACGCTGGTCATGAAATGACGAAGGCTATCGCCATTG
CACAGTTTAATGATGACAGCCCGGAAGCGAGGAAAATAACCCGGCGCTGGAGAATAGGTGAAGCAGCGGATTTAGTTGG
GGTTTCTTCTCAGGCTATCAGAGATGCCGAGAAAGCAGGGCGACTACCGCACCCGGATATGGAAATTCGAGGACGGGTT
GAGCAACGTGTTGGTTATACAATTGAACAAATTAATCATATGCGTGATGTGTTTGGTACGCGATTGCGACGTGCTGAAG
ACGTATTTCCACCGGTGATCGGGGTTGCTGCCCATAAAGGTGGCGTTTACAAAACCTCAGTTTCTGTTCATCTTGCTCA
GGATCTGGCTCTGAAGGGGCTACGTGTTTTGCTCGTGGAAGGTAACGACCCCCAGGGAACAGCCTCAATGTATCACGGA
TGGGTACCAGATCTTCATATTCATGCAGAAGACACTCTCCTGCCTTTCTATCTTGGGGAAAAGGACGATGTCACTTATG
CAATAAAGCCCACTTGCTGGCCGGGGCTTGACATTATTCCTTCCTGTCTGGCTCTGCACCGTATTGAAACTGAGTTAAT
GGGCAAATTTGATGAAGGTAAACTGCCCACCGATCCACACCTGATGCTCCGACTGGCCATTGAAACTGTTGCTCATGAC
TATGATGTCATAGTTATTGACAGCGCGCCTAACCTGGGTATCGGCACGATTAATGTCGTATGTGCTGCTGATGTGCTGA
TTGTTCCCACGCCTGCTGAGTTGTTTGACTACACCTCCGCACTGCAGTTTTTCGATATGCTTCGTGATCTGCTCAAGAA
CGTTGATCTTAAAGGGTTCGAGCCTGATGTACGTATTTTGCTTACCAAATACAGCAATAGCAATGGCTCTCAGTCCCCG
TGGATGGAGGAGCAAATTCGGGATGCCTGGGGAAGCATGGTTCTAAAAAATGTTGTACGTGAAACGGATGAAGTTGGTA
AAGGTCAGATCCGGATGAGAACTGTTTTTGAACAGGCCATTGATCAACGCTCTTCAACTGGTGCCTGGAGAAATGCTCT
TTCTATTTGGGAACCTGTCTGCAATGAAATTTTCGATCGTCTGATTAAACCACGCTGGGAGATTAGATAATGAAGCGTG
CGCCTGTTATTCCAAAACATACGCTCAATACTCAACCGGTTGAAGATACTTCGTTATCGACACCAGCTGCCCCGATGGT
GGATTCGTTAATTGCGCGCGTAGGAGTAATGGCTCGCGGTAATGCCATTACTTTGCCTGTATGTGGTCGGGATGTGAAG
TTTACTCTTGAAGTGCTCCGGGGTGATAGTGTTGAGAAGACCTCTCGGGTATGGTCAGGTAATGAACGTGACCAGGAGC
TGCTTACTGAGGACGCACTGGATGATCTCATCCCTTCTTTTCTACTGACTGGTCAACAGACACCGGCGTTCGGTCGAAG
AGTATCTGGTGTCATAGAAATTGCCGATGGGAGTCGCCGTCGTAAAGCTGCTGCACTTACCGAAAGTGATTATCGTGTT
CTGGTTGGCGAGCTGGATGATGAGCAGATGGCTGCATTATCCAGATTGGGTAACGATTATCGCCCAACAAGTGCTTATG
AACGTGGTCAGCGTTATGCAAGCCGATTGCAGAATGAATTTGCTGGAAATATTTCTGCGCTGGCTGATGCGGAAAATAT
TTCACGTAAGATTATTACCCGCTGTATCAACACCGCCAAATTGCCTAAATCAGTTGTTGCTCTTTTTTCTCACCCCGGT
GAACTATCTGCCCGGTCAGGTGATGCACTTCAAAAAGCCTTTACAGATAAAGAGGAATTACTTAAGCAGCAGGCATCTA
ACCTTCATGAGCAGAAAAAAGCTGGGGTGATATTTGAAGCTGAAGAAGTTATCACTCTTTTAACTTCTGTGCTTAAAAC
GTCATCTGCATCAAGAACTAGTTTAAGCTCACGACATCAGTTTGCTCCTGGAGCGACAGTATTGTATAAGGGCGATAAA
ATGGTGCTTAACCTGGACAGGTCTCGTGTTCCAACTGAGTGTATAGAGAAAATTGAGGCCATTCTTAAGGAACTTGAAA
AGCCAGCACCCTGATGCGACCACGTTTTAGTCTACGTTTATCTGTCTTTACTTAATGTCCTTTGTTACAGGCCAGAAAG
CATAACTGGCCTGAATATTCTCTCTGGGCCCACTGTTCCACTTGTATCGTCGGTCTGATAATCAGACTGGGACCACGGT
CCCACTCGTATCGTCGGTCTGATTATTAGTCTGGGACCACGGTCCCACTCGTATCGTCGGTCTGATTATTAGTCTGGGA
CCACGGTCCCACTCGTATCGTCGGTCTGATAATCAGACTGGGACCACGGTCCCACTCGTATCGTCGGTCTGATTATTAG
TCTGGGACCATGGTCCCACTCGTATCGTCGGTCTGATTATTAGTCTGGGACCACGGTCCCACTCGTATCGTCGGTCTGA
TTATTAGTCTGGAACCACGGTCCCACTCGTATCGTCGGTCTGATTATTAGTCTGGGACCACGGTCCCACTCGTATCGTC
GGTCTGATTATTAGTCTGGGACCACGATCCCACTCGTGTTGTCGGTCTGATTATCGGTCTGGGACCACGGTCCCACTTG
TATTGTCGATCAGACTATCAGCGTGAGACTACGATTCCATCAATGCCTGTCAAGGGCAAGTATTGACATGTCGTCGTAA
CCTGTAGAACGGAGTAACCTCGGTGTGCGGTTGTATGCCTGCTGTGGATTGCTGCTGTGTCCTGCTTATCCACAACATT
TTGCGCACGGTTATGTGGAC
```

### REFERENCES

1. Viacyte. Center for Beta Cell Therapy in Diabetes and ViaCyte Announce Start of European Clinical Trial of Human Stem Cell-derived Implants in Type 1 Diabetes Patients, <https://viacyte.com/archives/press-releases/center-for-beta-cell-therapy-in-diabetes-and-viacyte-announce-start-of-european-clinical-trial-of-human-stem-cell-derived-implants-in-type-1-diabetes-patients>(2019).
2. Qasim, W. Allogeneic CAR T cell therapies for leukemia. Am J Hematol 94, S50-S54, doi:10.1002/ajh.25399 (2019).
3. Nature. ‘Reprogrammed’ stem cells approved to mend human hearts for the first time, <https://www.nature.com/articles/d41586-018-05278-8>(2018).
4. Mandai, M. et al. Autologous Induced Stem-Cell-Derived Retinal Cells for Macular Degeneration. N Engl J Med 376, 1038-1046, doi:10.1056/NEJMoa1608368 (2017).
5. Nature. Japanese woman is first recipient of next-generation stem cells, <https://www.nature.com/news/japanese-woman-is-first-recipient-of-next-generation-stem-cells-1.15915>(2014).
6. Kikuchi, T. et al. Human iPS cell-derived dopaminergic neurons function in a primate Parkinson's disease model. Nature 548, 592-596, doi:10.1038/nature23664 (2017).
7. Science. First-of-its-kind clinical trial will use reprogrammed adult stem cells to treat Parkinson's, <https://www.sciencemag.org/news/2018/07/first-its-kind-clinical-trial-will-use-reprogrammed-adult-stem-cells-treat-parkinson-s>(2018).
8. Saudemont, A., Jespers, L. & Clay, T. Current Status of Gene Engineering Cell Therapeutics. Front Immunol 9, 153, doi:10.3389/fimmu.2018.00153 (2018).
9. Llore, N. P., Bruestle, K. A. & Griesemer, A. Xenotransplantation tolerance: applications for recent advances in modified swine. Curr Opin Organ Transplant 23, 642-648, doi:10.1097/MOT.0000000000000585 (2018).
10. Langin, M. et al. Consistent success in life-supporting porcine cardiac xenotransplantation. Nature, doi:10.1038/s41586-018-0765-z (2018).
11. Bravery, C. A. Do human leukocyte antigen-typed cellular therapeutics based on induced pluripotent stem cells make commercial sense? Stem Cells Dev 24, 1-10, doi:10.1089/scd.2014.0136 (2015).
12. Youssef, A. A. et al. The Promise and Challenge of Induced Pluripotent Stem Cells for Cardiovascular Applications. JACC Basic Transl Sci 1, 510-523, doi:10.1016/j.jacbts.2016.06.010 (2016).
13. Yoshihara, M., Hayashizaki, Y. & Murakawa, Y. Genomic Instability of iPSCs: Challenges Towards Their Clinical Applications. Stem Cell Rev 13, 7-16, doi:10.1007/s12015-016-9680-6 (2017).
14. Fujita, M. & Tabuchi, K. A rebuttal to Akabayashi and colleagues' criticisms of the iPSC stock project. J Med Ethics, doi:10.1136/medethics-2018-105248 (2019).
15. Okita, K. et al. A more efficient method to generate integration-free human iPS cells. Nat Methods 8, 409-412, doi:10.1038/nmeth.1591 (2011).
16. Lanza, R., Russell, D. W. & Nagy, A. Engineering universal cells that evade immune detection. Nat Rev Immunol, doi:10.1038/s41577-019-0200-1 (2019).
17. Gornalusse, G. G. et al. HLA-E-expressing pluripotent stem cells escape allogeneic responses and lysis by NK cells. Nat Biotechnol 35, 765-772, doi:10.1038/nbt.3860 (2017).
18. Cabrera, T. et al. High frequency of altered HLA class I phenotypes in laryngeal carcinomas. Hum Immunol 61, 499-506 (2000).
19. Cabrera, T. et al. High frequency of altered HLA class I phenotypes in invasive breast carcinomas. Hum Immunol 50, 127-134 (1996).
20. Beziat, V., Descours, B., Parizot, C., Debre, P. & Vieillard, V. NK cell terminal differentiation: correlated stepwise decrease of NKG2A and acquisition of KIRs. PLoS One 5, e11966, doi:10.1371/journal.pone.0011966 (2010).
21. Bjorkstrom, N. K. et al. Expression patterns of NKG2A, KIR, and CD57 define a process of CD56dim NK-cell differentiation uncoupled from NK-cell education. Blood 116, 3853-3864, doi:10.1182/blood-2010-04-281675 (2010).
22. Ye, H. & Fussenegger, M. Synthetic therapeutic gene circuits in mammalian cells. FEBS Lett 588, 2537-2544, doi:10.1016/j.febslet.2014.05.003 (2014).
23. Chakravarti, D. & Wong, W. W. Synthetic biology in cell-based cancer immunotherapy. Trends Biotechnol 33, 449-461, doi:10.1016/j.tibtech.2015.05.001 (2015).
24. Ho, P. & Chen, Y. Y. Mammalian synthetic biology in the age of genome editing and personalized medicine. Curr Opin Chem Biol 40, 57-64, doi:10.1016/j.cbpa.2017.06.003 (2017).
25. Meinke, G., Bohm, A., Hauber, J., Pisabarro, M. T. & Buchholz, F. Cre Recombinase and Other Tyrosine Recombinases. Chem Rev 116, 12785-12820, doi:10.1021/acs.chemrev.6b00077 (2016).
26. Kosicki, M., Tomberg, K. & Bradley, A. Repair of double-strand breaks induced by CRISPR-Cas9 leads to large deletions and complex rearrangements. Nat Biotechnol 36, 765-771, doi:10.1038/nbt.4192 (2018).
27. Rezza, A. et al. Unexpected genomic rearrangements at targeted loci associated with CRISPR/Cas9-mediated knock-in. Sci Rep 9, 3486, doi:10.1038/s41598-019-40181-w (2019).
28. Morishima, Y. et al. Risk of HLA Homozygous Cord Blood Transplantation: Implications for Induced Pluripotent Stem Cell Banking and Transplantation. Stem Cells Transl Med 7, 173-179, doi:10.1002/sctm.17-0169 (2018).
29. Nakatsuji, N., Nakajima, F. & Tokunaga, K. HLA-haplotype banking and iPS cells. Nat Biotechnol 26, 739-740, doi:10.1038/nbt0708-739 (2008).
30. Taylor, C. J., Peacock, S., Chaudhry, A. N., Bradley, J. A. & Bolton, E. M. Generating an iPSC bank for HLA-matched tissue transplantation based on known donor and recipient HLA types. Cell Stem Cell 11, 147-152, doi:10.1016/j.stem.2012.07.014 (2012).
31. Kurtzberg, J. et al. Results of the Cord Blood Transplantation Study (COBLT): clinical outcomes of unrelated donor umbilical cord blood transplantation in pediatric patients with hematologic malignancies. Blood 112, 4318-4327, doi:10.1182/blood-2007-06-098020 (2008).
32. Johnson, R. J. et al. Factors influencing outcome after deceased heart beating donor kidney transplantation in the United Kingdom: an evidence base for a new national kidney allocation policy. Transplantation 89, 379-386, doi:10.1097/TP.0b013e3181c90287 (2010).
33. Opelz, G. & Dohler, B. Pediatric kidney transplantation: analysis of donor age, HLA match, and posttransplant non-Hodgkin lymphoma: a collaborative transplant study report. Transplantation 90, 292-297, doi:10.1097/TP.0b013e3181e46a22 (2010).
34. Frequencies, A. HLA alelle frequencies webiste, <http://www.allelefrequencies.net/>(
35. Truong, D. M., Kaler, G., Khandelwal, A., Swaan, P. W. & Nigam, S. K. Multi-level analysis of organic anion transporters 1, 3, and 6 reveals major differences in structural determinants of antiviral discrimination. J Biol Chem 283, 8654-8663, doi:10.1074/jbc.M708615200 (2008).
36. Truong, D. M., Hewitt, F. C., Hanson, J. H., Cui, X. & Lambowitz, A. M. Retrohoming of a Mobile Group II Intron in Human Cells Suggests How Eukaryotes Limit Group II Intron Proliferation. PLoS Genet 11, e1005422, doi:10.1371/journal.pgen.1005422 (2015).
37. Truong, D. M., Sidote, D. J., Russell, R. & Lambowitz, A. M. Enhanced group II intron retrohoming in magnesium-deficient Escherichia coli via selection of mutations in the ribozyme core. Proc Natl Acad Sci USA 110, E3800-3809, doi:10.1073/pnas.1315742110 (2013).
38. Truong, D. M. & Boeke, J. D. Resetting the Yeast Epigenome with Human Nucleosomes. Cell 171, 1508-1519 e1513, doi:10.1016/j.cell.2017.10.043 (2017).
39. Richardson, S. M. et al. Design of a synthetic yeast genome. Science 355, 1040-1044, doi:10.1126/science.aaf4557 (2017).
40. Mitchell, L. A. et al. Synthesis, debugging, and effects of synthetic chromosome consolidation: synVI and beyond. Science 355, doi:10.1126/science.aaf4831 (2017).
41. Mitchell, L. A. et al. Versatile genetic assembly system (VEGAS) to assemble pathways for expression in S. cerevisiae. Nucleic Acids Res 43, 6620-6630, doi:10.1093/nar/gkv466 (2015).
42. Mitchell, L. A. et al. De novo assembly, delivery and expression of a 101 kb human gene in mouse cells. bioRxiv (2018).
43. Mitchell, L. A. et al. qPCRTag Analysis—A High Throughput, Real Time PCR Assay for Sc2.0 Genotyping. J Vis Exp, e52941, doi:10.3791/52941 (2015).
44. https://bethematch.org/patients-and-families/before-transplant/find-a-donor/hla-matching/. <https://bethematch.org/patients-and-families/before-transplant/find-a-donor/hla-matching/>(
45. Solomon, S., Pitossi, F. & Rao, M. S. Banking on iPSC—is it doable and is it worthwhile. Stem Cell Rev 11, 1-10, doi:10.1007/s12015-014-9574-4 (2015).
46. Byrne, S. M., Ortiz, L., Mali, P., Aach, J. & Church, G. M. Multi-kilobase homozygous targeted gene replacement in human induced pluripotent stem cells. Nucleic Acids Res 43, e21, doi:10.1093/nar/gku1246 (2015).
47. Renaud, J. B. et al. Improved Genome Editing Efficiency and Flexibility Using Modified Oligonucleotides with TALEN and CRISPR-Cas9 Nucleases. Cell Rep 14, 2263-2272, doi:10.1016/j.celrep.2016.02.018 (2016).
48. Inscripta. MAD7 commercial terms, <https://www.inscripta.com/madzymes/free-faq/>(2019).
49. Iacovino, M. et al. Inducible cassette exchange: a rapid and efficient system enabling conditional gene expression in embryonic stem and primary cells. Stem Cells 29, 1580-1588 (2011).
50. Institute, C. PGP1 iPSC line derived from Dr. George Church, <https://www.coriell.org/O/Sections/Search/Sample Detail.aspx?Ref=GM23338&PgId=166>(2019).
51. Bevan, M. J. The earliest knockouts. J Immunol 184, 4585-4586, doi:10.4049/jimmuno1.1090023 (2010).
52. Rene, C., Lozano, C. & Eliaou, J. F. Expression of classical HLA class I molecules: regulation and clinical impacts: Julia Bodmer Award Review 2015. HLA 87, 338-349, doi:10.1111/tan.12787 (2016).
53. Cereb, N. & Yang, S. Y. The regulatory complex of HLA class I promoters exhibits locus-specific conservation with limited allelic variation. J Immunol 152, 3873-3883 (1994).
54. Fishilevich, S. et al. GeneHancer: genome-wide integration of enhancers and target genes in GeneCards. Database (Oxford) 2017, doi:10.1093/database/bax028 (2017).
55. Noe, L. & Kucherov, G. YASS: enhancing the sensitivity of DNA similarity search. Nucleic Acids Res 33, W540-543, doi:10.1093/nar/gki478 (2005).
56. Wallace, H. A. et al. Manipulating the mouse genome to engineer precise functional syntenic replacements with human sequence. Cell 128, 197-209, doi:10.1016/j.ce11.2006.11.044 (2007).
57. Sachamitr, P., Leishman, A. J., Davies, T. J. & Fairchild, P. J. Directed Differentiation of Human Induced Pluripotent Stem Cells into Dendritic Cells Displaying Tolerogenic Properties and Resembling the CD141(+) Subset. Front Immunol 8, 1935, doi:10.3389/fimmu.2017.01935 (2017).
58. H. A. Wallace et al., Manipulating the mouse genome to engineer precise functional syntenic replacements with human sequence. Cell 128, 197-209 (2007).
59. L. A. Mitchell et al., De novo assembly, delivery and expression of a 101 kb human gene in mouse cells. Genetics. 2021 May 17;218(1):iyab038. doi: 10.1093/genetics/iyab038.
60. E.C. Lee, Q Liang et al., Complete humanization of the mouse immunoglobulin loci enables efficient therapeutic antibody discovery. Nat Biotechnol 32, 356-363 (2014). https://doi.org/10.1038/nbt.2825
61. R Brosh, Laurent J M et al., A versatile platform for locus-scale genome rewriting and verification. PNAS (2021) 118 (10) e2023952118 https://doi.org/10.1073/pnas.2023952118
62. Pinglay, et al., Synthetic genomic reconstitution reveals principles of mammalian Hox cluster regulation. BioRxiv. (2021) https://doi.org/10.1101/2021.07.07.451065
63. Shrikant Mali. Delivery systems for gene therapy. Indian J Hum Genet. 2013 January;19(1):3-8. doi: 10.4103/0971-6866.112870.
64. Rosa, et al., Direct programming of fibroblasts into antigen presenting dendritic cells. Sci Immunol. 2018 Dec. 7; 3(30):eaau4292. doi: 10.1126/sciimmunol.aau4292.
65. Laumont, et al., Noncoding regions are the main source of targetable tumor-specific antigens. Sci Transl Med. 2018 Dec. 5; 10(470):eaau5516. doi: 10.1126/scitranslmed.aau5516.
66. Zhang, et al., A Ubiquitous Chromatin Opening Element (UCOE) Confers Resistance to DNA Methylation-mediated Silencing of Lentiviral Vectors. Mol Ther. 2010 September; 18(9): 1640-1649.

47.-111. (canceled)

112. A method of integrating a DNA construct into a genome, the method comprising:
a. contacting the genome from a sample with one or more agents, wherein the one or more agents are capable of cleaving the genome at a locus;
b. integrating a first nucleic acid sequence into the genome at the locus; and
c. integrating one or more second nucleic acid sequences into the first nucleic acid sequence in the genome, thereby integrating the DNA construct into the genome, wherein each of the one or more second nucleic acid sequences comprises a cargo sequence.

113. The method of claim 112, wherein the cargo sequence comprises at least 1 kilobase.

114. The method of claim 112, wherein the locus comprises a human genomic locus comprising Human Leukocyte Antigens (HLA), Adeno-Associated Virus Integration Site 1 (AAVS1), C-C Motif Chemokine Receptor type 5 (CCR5), human Reverse Orientation Splice Acceptor 26 (hROSA26), Cytochrome B-245 Beta Chain (CYBB), Cluster of Differentiation 40 Ligand (CD40LG), Cyclin-Dependent Kinase 6 (CDK6), Toll-Like Receptor (TLR8), T Cell Receptor Beta Constant 1 (TRBC1), Hypoxanthine Phosphoribosyltransferase (HPRT), or Homeobox A cluster (HOXA).

115. The method of claim 112, wherein the locus comprises murine Reverse Orientation Splice Acceptor 26 (mROSA26).

116. The method of claim 112, wherein the first nucleic acid sequence comprises a landing pad sequence, wherein the landing pad sequence comprises one or more genes, regulatory elements, or combinations thereof.

117. The method of claim 116, wherein the one or more genes comprise a gene encoding estrogen receptor 2 (ERT2) or a variant thereof, a gene encoding P2A or a variant thereof, a gene encoding a Thymidine Kinase or a variant thereof, a gene encoding a recombinase or a variant thereof, a blasticidin resistance gene, a hygromycin resistance gene, or a gene encoding a fluorescent protein or a variant thereof.

118. The method of claim 117, wherein the ERT2 gene or the variant thereof is fused with a gene encoding a recombinase or a variant thereof, wherein the recombinase or the variant thereof comprises a tyrosine recombinase, a Cre recombinase, a Flp recombinase, a variant thereof, or a combination thereof, wherein the recombinase is codon-optimized for mammals, and/or wherein the recombinase lacks CpG sites.

119. The method of claim 117, wherein (i) expression of the ERT2 gene or the variant thereof is induced by tamoxifen; and/or (ii) expression of the gene encoding the recombinase or the variant thereof is induced by a gene expression system or doxycycline.

120. The method of claim 117, wherein the fluorescent protein comprises a red fluorescent protein, a green fluorescent protein, a yellow fluorescent protein, a blue fluorescent protein, or a cyan fluorescent protein.

121. The method of claim 116, wherein the one or more regulatory elements comprise a promoter, a terminator, a recombinase recognition site, or a combination thereof, wherein (i) the promoter comprises a universal chromatin opening element (UCOE), a human EF1α promoter, a phosphoglycerate kinase (PGK), or a combination thereof, (ii) the terminator comprises a simian virus 40 (SV40) terminator, a bovine growth hormone (bGH) terminator, a human growth hormone (hGH) terminator, a rabbit beta-globin (rbGlob), or a combination thereof, (iii) the recombinase recognition site comprises a Cre recognition site, a Flp recognition site, or a combination thereof, and/or (iv) the recombinase recognition site comprises one or more of a loxP or a variant thereof, a loxM or a variant thereof, a lox2722 or a variant thereof, a loxFAS or a variant thereof, a lox5171 or a variant thereof, a frt or a variant thereof, or a combination thereof.

122. The method of claim 116, wherein the landing pad sequence comprises a sequence comprising SEQ ID NO: 2, SEQ ID NO: 5, SEQ ID NO: 6, or SEQ ID NO: 7.

123. The method of claim 112, wherein the one or more second nucleic acid sequences are provided by one or more vectors configured to carry nucleic acid sequence that comprises approximately from 10 kilobases to 1,000 kilobases.

124. The method of claim 123, wherein the one or more vectors comprise a marker, wherein the marker is devoid of a promoter and a start codon, and/or wherein the marker comprises a selectable marker comprising an antibiotic marker.

125. The method of claim 112, wherein the genome in the sample is contained in a cell, wherein the contacting in step a) and the integrating in steps b) and c) are performed in the cell.

126. The method of claim 125, wherein the cell comprises a mammalian cell comprising a human cell or a mouse cell; or a primary cell comprising an embryonic stem cell (ESC), a pluripotent stem cell (PSC), or an induced pluripotent stem cell (iPSC).

127. The method of claim 112, wherein the one or more agents comprise a polypeptide, a polynucleotide, or a combination thereof,
wherein (i) the polypeptide comprises a site-specific nuclease comprising an engineered homing endonuclease or meganuclease, a zinc-finger nuclease (ZFN), a transcription activator-like effector nuclease (TALEN), a clustered regularly interspaced short palindromic repeat (CRISPR), or a combination thereof, and/or (ii) the polynucleotide comprises a guide RNA (gRNA).

128. The method of claim 112, wherein the one or more agents are delivered via a carrier comprising a vector, a messenger RNA (mRNA), a double stranded DNA (dsDNA), a single stranded DNA (ssDNA), or a plasmid.

129. The method of claim 112, wherein the sample comprises a biological sample comprising a cell, tissue sample, or blood sample, wherein the sample is obtained from a subject comprising a human.

130. The method of claim 112, wherein the integrating in steps b) and c) comprises sequential integration.

131. The method of claim 112, wherein the integrating in steps b) and c) comprises homology-directed recombination (HR), and wherein the integrating comprises utilizing an inducible or constitutive site-specific recombinase Cre, a Flp recombinase, a self-excising recombinase, or a combination thereof.
